(19) United States
(12) Patent Application Publication
(10) Pub. No.: US 2026/0001845 A1
(43) Pub. Date: Jan. 1, 2026

(54) MITOGEN-ACTIVATED PROTEIN KINASE (MEK) INHIBITORS

(71) Applicant: Nested Therapeutics. Inc., Cambridge, MA (US)

(72) Inventors: Yongxin Han, Cambridge, MA (US); Michael Hale, Cambridge, MA (US); David Belanger, Cambridge, MA (US); Mark E. Fitzgerald, Cambridge, MA (US); Jeffrey Hale, Cambridge, MA (US); Daniel F. Ortwine, Cambridge, MA (US); Aysegul Ozen, Cambridge, MA (US)

(21) Appl. No.: 18/992,070

(22) PCT Filed: Jul. 7, 2023
(86) PCT No.: PCT/US2023/027122

§ 371 Date: Jan. 7, 2025

Related U.S. Application Data
(60) Provisional application No. 63/359,537, filed on Jul. 8, 2022.

(51) Int. Cl. C07D 213/76; A61K 31/44; A61K 31/444; A61K 31/4709; A61K 31/497; A61K 31/501; A61K 31/506; C07B 59/00; C07D 213/74; C07D 213/75; C07D 213/89; C07D 401/06; C07D 401/14; C07D 405/14; C07D 417/14; C07D 471/04; C07D 491/056

(52) U.S. Cl. CPC C07D 213/76; A61K 31/44; A61K 31/444; A61K 31/4709; A61K 31/497; A61K 31/501; A61K 31/506; C07B 59/002; C07D 213/74; C07D 213/75; C07D 213/89; C07D 401/06; C07D 401/14; C07D 405/14; C07D 417/14; C07D 471/04; C07D 491/056

(57) ABSTRACT

The present invention is related to compounds of structure (F) as mitogen-activated protein kinase (MEK) inhibitors. (I) The variables are described herein.

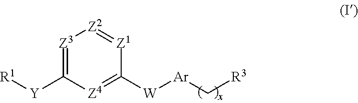

### RELATED APPLICATIONS

This application claims the benefit of U.S. Provisional Application Ser. No. 63/359,537, filed Jul. 8, 2022, the entire teachings of which are incorporated herein by reference.

### BACKGROUND OF THE INVENTION

Cancer is among the most common causes of death in the United States. In the United States, cancer has accounted for approximately one of every four deaths. The 5-year relative survival rate for cancer patients diagnosed in 1996-2003 is approximately two-thirds, up from about one half in 1975-1977 (Cancer Facts & Figures, American Cancer Society: Atlanta, Ga. (2008)). The rate of new cancer cases decreased by an average 0.6% per year among men between 2000 and 2009, but stayed the same for women. From 2000 through 2009, death rates from all cancers combined decreased on average 1.8% per year among men and 1.4% per year among women. This improvement in survival reflects progress in diagnosing at an earlier stage as well as improvements in treatment, for which there remain a need. Discovering highly effective anticancer agents with low toxicity is a primary goal of cancer research.

MEK is a critical signaling intermediate in the MAPK/ERK pathway, which is inappropriately activated across a broad spectrum of human tumors, including those derived from lung, pancreas, ovary, skin and colon. While several MEK inhibitors have achieved regulatory approval to date, these MEK inhibitors have yet to deliver against clinical efficacy expectations, and combination of these MEK inhibitors with RAF inhibitors are required to achieve more durable responses. Indentification of a new class of MEK inhibitors that can achieve dual inhibition of MEK/RAF and MEK/KSR can maximize pathologic reversal due to more complete suspression of the MAPK/ERK pathway, preventing paradoxical pathway reactivation while limiting drug-related toxicity would have a significant impact on cancer patient morbidity and mortality.

### SUMMARY OF THE INVENTION

Disclosed herein are novel inhibitors of mitogen-activated protein kinase (MEK), and extra cellular signal-regulated kinases (ERK) (see Example 105) and thus may be useful to treat cancers. The disclosed inhibitors have increased central nervous system penetration (CNS), and, as such, are expected to be useful in treating metastsis to the CNS, and CNS cancers.

In one embodiment, provided herein is a compound represented by structural Formula (I):

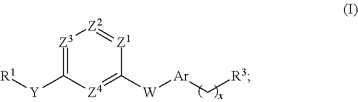

or a pharmaceutically acceptable salt thereof. The definition of each variable is provided below.

Pharmaceutical compositions of the compounds of the invention are also disclosed herein. Particular embodiments comprise a pharmaceutically acceptable carrier or diluent and one or more of the compounds of the invention, or a pharmaceutically acceptable salt thereof.

Another embodiment of the present invention is a method of inhibiting mitogen-activated protein kinase (MEK) or extra cellular signal-regulated kinases (ERK) in a subject in need thereof. The method comprises administering to the subject an effective amount of a compound disclosed herein or a pharmaceutical composition disclosed herein. In one example, a “subject in need thereof” is a subject with cancer.

### DETAILED DESCRIPTION

### Compounds of the Invention

In a first embodiment, the invention provides a compound represented by structural formula (I′):

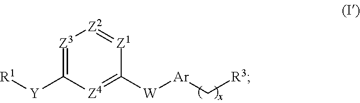

or a pharmaceutically acceptable salt thereof, wherein:
   Y is a covalent bond, NH, NCH₃, S, CH₂, OCH₂\hat{ } or O, wherein “\hat{ }” indicates the point of attachment to R¹;
   W is CH₂, CH(CH₃) or O;
   Z¹, Z² and Z³ are each independently selected from N, N-oxide and CR^{2a}, provided that no more than one of Z¹, Z² and Z³ is an N-oxide;
   Z⁴ is slected from N or CR^{2b}
   Ar is phenyl, a six to membered heteroaryl or 2-pyridinone, wherein the phenyl, the six membered heteroaryl, and 2-pyridinone are each independently substituted with zero, one or two groups represented by R⁴ and wherein

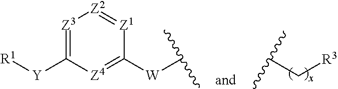

are 1,3 or 1,4 relative to each other on the group represented by Ar;
R¹ is, C₁₋₆ alkyl, C₂₋₆ alkenyl, C₂₋₆ alkynyl, C₁₋₆ haloalkyl, pyridinonyl, C₃₋₆ cycloalkyl, phenyl, a 5-10 membered heteroaryl or C(O)N(R⁶)₂, wherein the C₃₋₆ cycloalkyl, phenyl, and the 5-10 membered heteroaryl, are each independently substituted with zero, one, two or three groups represented by R⁵;
R^{2a} is H, F or C₁₋₃ alkyl;
R^{2b} is H, halo, (CH₂)ₙOR⁷, C₁₋₆ alkyl, C₂₋₆ alkenyl, C₂₋₆ alkynyl, C₁₋₆ haloalkyl, C₁₋₆ alkoxy, C(O)N(C₁₋₆-alkyl), C(O)NHO(C₂₋₆ hydroxyalkyl), (CH₂)₂₋₆N(R⁷)₂, C(O)NHO(CH₂)₂₋₆N(R⁷)₂, C₃₋₆ cycloalkyl, phenyl, a 5-6 membered heteroaryl or 4-6 membered heterocycle; or R^{2b} and Y taken together with their intervening atoms form a 5-6 membered nitrogen containing heterocycle or a 5-6 membered nitrogen containing heteroaryl; and
R³ is N(R¹⁰)₂,

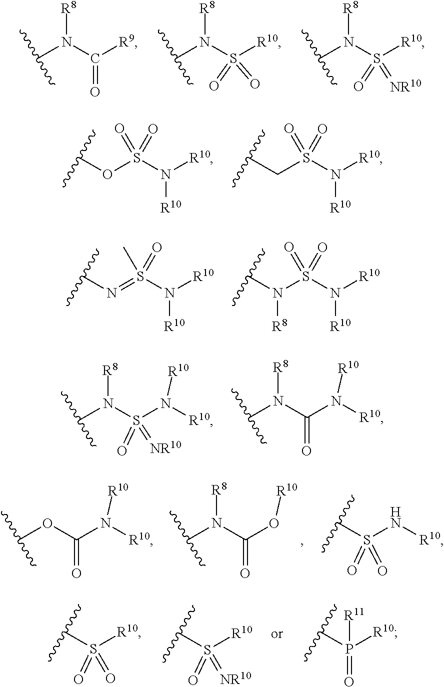

each R⁴ is independently H, halo, C₁₋₆ alkoxy or C₁₋₆ alkyl;
R⁵ is H, cyano, halo, SO₂ C₁₋₆ alkyl, C₁₋₆ alkyl, deuterated C₁₋₆ alkyl, C₂₋₆ alkenyl, deuterated C₁₋₆ alkenyl, C₂₋₆ alkynyl, deuterated C₁₋₆ alkynyl, C₁₋₆ haloalkyl, C₁₋₆ alkoxy, C₁₋₆ haloalkoxy, SC₁₋₆ alkyl, C₃₋₈ cycloalkyl; or two R⁵s on adjacent phenyl ring carbon atoms taken together with the ring carbon atoms to which they are attached form an oxygen containing heterocycle; or two R's on the same ring carbon atom of a C₃₋₆ cycloalkyl form a 4-6 membered nitogen containing heterocycyle optionally substituted with C₁₋₄ alkyl; and
each R⁶ is independently selected from H or C₁₋₆ alkyl (preferably H or C₁₋₆ alkyl);
each R⁷ and each R⁸ are independently selected from H or C₁₋₃ alkyl; or when x is 0, R⁸ and an R⁴ ortho to W and R³ taken together with their intervening atoms form a 5-6 membered nitrogen containing heterocycle;
R⁹ is H, C₁₋₆ alkoxy, C₁₋₆ alkyl, C₂₋₆ alkenyl, C₁₋₆ haloalkyl, C₃₋₈ cycloalkyl (optionally substituted with methyl) or N(R¹¹)₂ wherein the C₁₋₆ alkyl is optionally substituted with cyano, hydroxy, C₁₋₆ alkoxy or N(R¹¹)₂;
each R¹⁰ is independently H, C₁₋₆ alkyl, C₂₋₆ alkenyl, C₃₋₈ cycloalkyl (optionally substituted with methyl) or C₁₋₆ haloalkyl, wherein the C₁₋₆ alkyl is optionally substituted with cyano, hydroxy, C₁₋₆ alkoxy or N(R¹¹)₂; or two R¹⁰s taken together with the nitrogen atom to which they are bonded form a 3-7 membered heterocycle;
each R¹¹ is independently H or methyl;
n is 0 or 1; and
x is 0 or 1.

In a second embodiment, the invention provides a compound represented by structural formula (I):

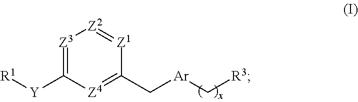

or a pharmaceutically acceptable salt thereof, wherein:
Z¹, Z² and Z³ are each independently selected from N and CR^{2a};
R¹ is, C₁₋₆ alkyl, C₂₋₆ alkenyl, C₂₋₆ alkynyl, C₁₋₆ haloalkyl, C₃-6 cycloalkyl, phenyl, a 5-6 membered heteroaryl or C(O)N(R⁶)₂, wherein the phenyl, and the 5-6 membered heteroaryl, are each independently substituted with zero, one or two groups represented by R⁵;
R^{2b} is H, halo, (CH₂)ₙOR⁷, C₁₋₆ alkyl, C₂₋₆ alkenyl, C₂₋₆ alkynyl, C₁₋₆ haloalkyl, C₁₋₆ alkoxy, C₃₋₆ cycloalkyl, phenyl, a 5-6 membered heteroaryl or 4-6 membered heterocycle;
R³ is N(R¹⁰)₂,

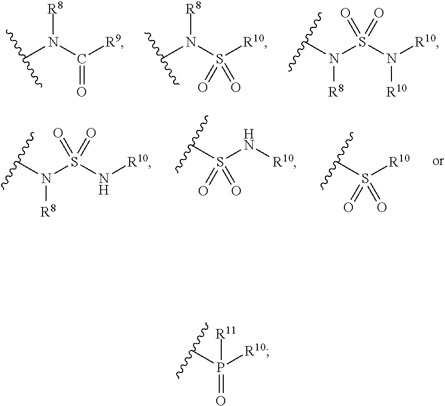

R⁵ is H, cyano, halo, C₁₋₆ alkyl, C₂₋₆ alkenyl, C₂₋₆ alkynyl, C₁₋₆ haloalkyl, C₁₋₆ alkoxy, halomethoxy or C₃₋₈ cycloalkyl;
R⁷ and R⁸ are independently selected from H or C₁₋₃ alkyl; and the remainder of the variables are as described in the first embodiment.

In a third embodiment, the invention provides a compound represented by structural formula (I′) or a pharmaceutically acceptable salt thereof, wherein R¹ is C₁₋₄ alkyl, C₂₌₄ alkenyl, C₂₋₄ alkynyl,

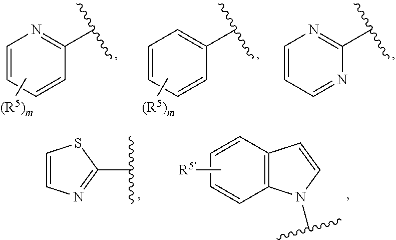

C₄-C₆ cycloalkyl optionally substituted with one or two R⁵″ or C(O)N(R⁶)₂; R⁵′ is H or halo; each R⁵″ is C₁₋₃ alkyl or two R⁵″ taken together with the ring carbon atom to which they are bonded form a C₄₋₆ nitrogen containing heterocyclyl wherein the ring nitrogen atom is optionally N—(C₁₋₃) alkylated; and m is 0, 1 or 2.

Alternatively, R¹ is

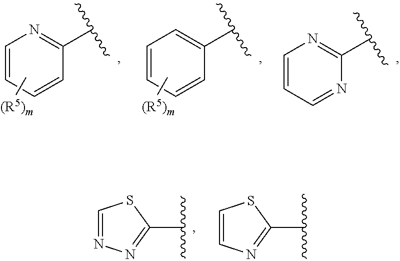

or C(O)N(R⁶)₂; and m is 0, 1 or 2. The remainder of the variables in both alternatives are as described in the first embodiment.

In a fourth embodiment, the invention provides a compound represented by structural formula (I) or a pharmaceutically acceptable salt thereof, wherein R¹ is

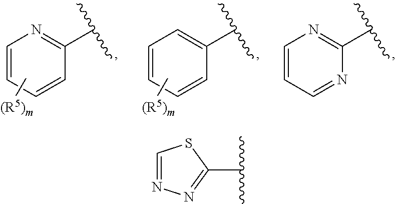

or C(O)N(R⁶)₂, m is 0, 1 or 2; and the remainder of the variables are as described in the second embodiment.

In a fifth embodiment, the invention provides a compound selected from (II), (IIa), (IIb), and (IIc):

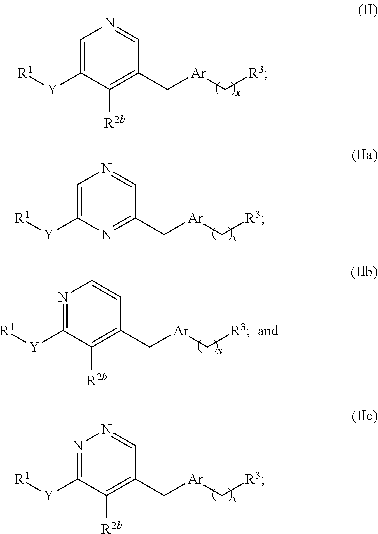

or a pharmaceutically acceptable salt thereof, wherein the the variables are as defined in the first, second, third or fourth embodiment.

In a sixth embodiment, the invention provides a compound represented by structural formula (II):

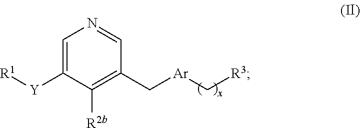

or a pharmaceutically acceptable salt thereof, wherein the the variables are as defined in the second or fourth embodiment.

In a seventh embodiment, the invention provides a compound represented by structural formula (I′), (I), (II), (IIa), (IIb), or (IIc) or a pharmaceutically acceptable salt thereof, wherein R¹ is methyl, allyl, propargyl,

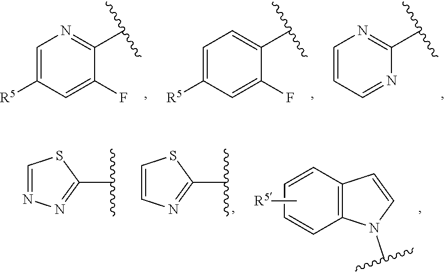

cyclohexyl optionally substituted by one or two methyl or C(O)N(R⁶)₂. Alternatively, R¹ is

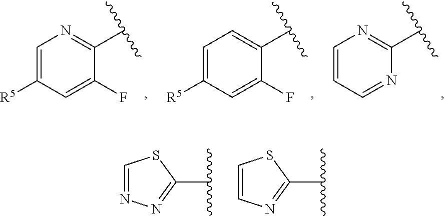

or C(O)N(R⁶)₂. The remainder of the variables in both alternatives are as described in the first, third, fifth or sixth embodiment.

In an eighth embodiment, the invention provides a compound represented by structural formula (I), (II), (IIa), (IIb), or (IIc) or a pharmaceutically acceptable salt thereof, wherein R¹ is

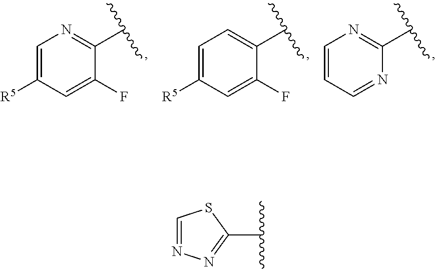

or C(O)N(R⁶)₂; and the remainder of the variables are as described in the second, fourth or sixth embodiment.

In a ninth embodiment, the invention provides a compound represented by structural formula (I′), (I), (II), (IIa), (IIb), or (IIc) or a pharmaceutically acceptable salt thereof, wherein Ar—(CH₂)ₓ—R³ is represented by the following structural formula:

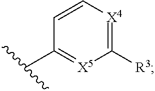

wherein X⁴ is N, CH, C(C₁₋₄alkyl) or C(C₁₋₄alkoxy) and X⁵ is N or CR⁴; and the remainder of the variables are as defined in the first, second, third, fourth, fifth, sixth, seventh or eighth embodiment.

In a tenth embodiment, the invention provides a compound represented by structural formula (III):

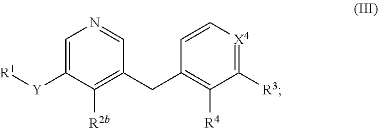

or a pharmaceutically acceptable salt thereof, wherein X⁴ is N or CH, and the remainder of the variables are as defined in the second, fourth or eighth embodiment.

In an eleventh embodiment, the invention provides a compound represented by structural formula (IV):

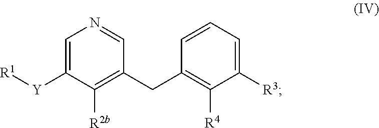

or a pharmaceutically acceptable salt thereof, wherein the variables are as defined in the second, fourth or eighth embodiment.

In a twelfth embodiment, the invention provides a compound represented by structural formula (V):

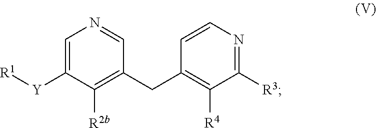

or a pharmaceutically acceptable salt thereof, wherein the variables are as defined in the second, fourth or eighth embodiment.

In a thirteenth embodiment, the invention provides a compound represented by structural formula (VI):

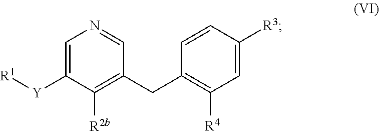

or a pharmaceutically acceptable salt thereof, wherein the variables are as defined in the second, fourth or eighth embodiment.

In a fourteenth embodiment, the invention provides a compound represented by structural formula (I′), (I), (II), (IIa), (IIb), or (IIc) or a pharmaceutically acceptable salt thereof, wherein Ar—(CH₂)ₓ—R³ is represented by the following structural formula selected from:

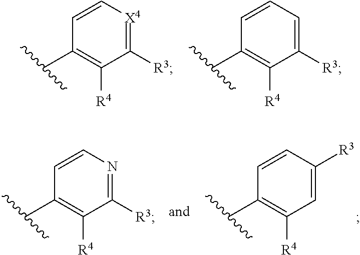

wherein the remainder of the variables are as defined in the first, second, third, fourth, fifth, sixth, seventh, eighth or ninth embodiment.

In a fifteenth embodiment, the invention provides a compound represented by structural formula (I′), (I), (II), (IIa), (IIb), (IIc), (III), (IV) (V) or (VI), or a pharmaceutically acceptable salt thereof, wherein R¹ is

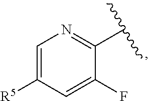

and the remainder of the variables are as defined in the first, second, third, fourth, fifth, sixth, seventh, eighth, ninth, tenth, eleventh, twelfth, thirteenth or fourteenth embodiment.

In a sixteenth embodiment, the invention provides a compound represented by structural formula (I′), (I), (II), (IIa), (IIb), (IIc), (III), (IV) (V) or (VI), or a pharmaceutically acceptable salt thereof, wherein R¹ is

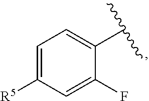

and the remainder of the variables are as defined in the first, second, third, fourth, fifth, sixth, seventh, eighth, ninth, tenth, eleventh, twelfth, thirteenth or fourteenth embodiment.

In a seventeenth embodiment, the invention provides a compound represented by structural formula (I′), (I), (II), (IIa), (IIb), (IIc), (III), (IV) (V) or (VI), or a pharmaceutically acceptable salt thereof, wherein R¹ is

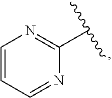 [SMILES: *c1ncccn1]

and the remainder of the variables are as defined in the first, second, third, fourth, fifth, sixth, seventh, eighth, ninth, tenth, eleventh, twelfth, thirteenth or fourteenth embodiment.

In an eighteenth embodiment, the invention provides a compound represented by structural formula (I′), (I), (II), (IIa), (IIb), (IIc), (III), (IV) (V) or (VI), or a pharmaceutically acceptable salt thereof, wherein R¹ is

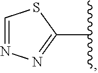 [SMILES: *c1nncs1]

and the remainder of the variables are as defined in the first, second, third, fourth, fifth, sixth, seventh, eighth, ninth, tenth, eleventh, twelfth, thirteenth or fourteenth embodiment.

In a nineteenth embodiment, the invention provides a compound represented by structural formula (I′), (I), (II), (IIa), (IIb), (IIc), (III), (IV) (V) or (VI), or a pharmaceutically acceptable salt thereof, wherein R¹ is C(O)N(R⁶)₂, wherein R⁶ is H or C₁₋₆ alkyl, preferably H or methyl, and the remainder of the variables are as defined in the first, second, third, fourth, fifth, sixth, seventh, eighth, ninth, tenth, eleventh, twelfth, thirteenth or fourteenth embodiment.

In a twentieth embodiment, the invention provides a compound represented by structural formula (I′), (I), (II), (IIa), (IIb), (IIc), (III), (IV) (V) or (VI), or a pharmaceutically acceptable salt thereof, wherein x is 0 and R³ is

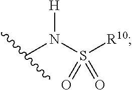

and the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-first embodiment, the invention provides a compound represented by structural formula (I′), (I), (II), (IIa), (IIb), (IIc), (III), (IV) (V) or (VI), or a pharmaceutically acceptable salt thereof, wherein x is 0 and R³ is

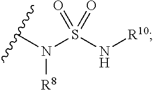

and the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-second embodiment, the invention provides a compound represented by structural formula (I′), (I), (II), (IIa), (IIb), (IIc), (III), (IV) (V) or (VI), or a pharmaceutically acceptable salt thereof, wherein x is 0 and R³ is

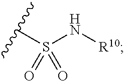

and the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-third embodiment, the invention provides a compound represented by structural formula (I′), (I), (II), (IIa), (IIb), (IIc), (III), (IV) (V) or (VI), or a pharmaceutically acceptable salt thereof, wherein x is 0 and R³ is

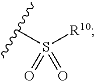

and the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-fourth embodiment, the invention provides a compound represented by structural formula (I′), (I), (II), (IIa), (IIb), (IIc), (III), (IV) (V) or (VI), or a pharmaceutically acceptable salt thereof, wherein x is 0 and R³ is

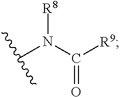

and the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth or nineteenth embodiment.

In a twenty-fifth embodiment, the invention provides a compound represented by structural formula (I), (II), (IIa), (IIb), (IIc), (III), (IV) (V) or (VI), or a pharmaceutically acceptable salt thereof, wherein Y is O and the remainder of the variables are as defined in the second, fourth, sixth, eighth, tenth, eleventh, twelfth or thirteenth embodiment.

In a twenty-sixth embodiment, the invention provides a compound represented by structural formula (I), (II), (IIa), (IIb), (IIc), (III), (IV) (V) or (VI), or a pharmaceutically acceptable salt thereof, wherein Y is NH and the remainder of the variables are as defined in the second, fourth, sixth, eighth, tenth, eleventh, twelfth or thirteenth embodiment.

In a twenty-seventh embodiment, the invention provides a compound represented by structural formula (I′), (I), (II), (IIa), (IIb), (IIc), (III), (IV) (V) or (VI), or a pharmaceutically acceptable salt thereof, wherein Y is O, NH, \hat{ }CH₂O, N(CH₃) or S or Y taken together with R^{2b} forms NHCH═CH or NHCH₂CH₂CH₂, wherein the “\hat{ }” indicates the point of attachment to R¹. Alternatively, Y is O, NH, N(CH₃) or S. The remainder of the variables in both alternatives are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment.

In a twenty-eighth embodiment, the invention provides a compound represented by structural formula (I), (II), (IIa), (IIb), (IIc), (III), (IV) (V) or (VI), or a pharmaceutically acceptable salt thereof, wherein R⁸ is H, R⁹ is C₁₋₆ alkoxy, C₁₋₆ alkyl, or N(R¹¹)₂ and R¹⁰ is C₁-C₆ alkyl and the remainder of the variables are as defined in the second, fourth, sixth, eighth, tenth, eleventh, twelfth, thirteenth, fifteenth, sixteenth, seventeenth, eighteenth, nineteenth, twentieth, twenty-first, twenty-second, twenty-third or twenty-fourth embodiment

In a twenty-ninth embodiment, the invention provides a compound represented by structural formula (I′), (I), (II), (IIa), (IIb), (IIc), (III), (IV) (V) or (VI), or a pharmaceutically acceptable salt thereof, wherein R^{2b} is H, C₁₋₆ alkyl, halo, C₁₋₆ alkoxy, (CH₂)ₙOR⁷ or 4-6 membered heterocycle; R⁴ is H, C₁₋₆ alkoxy or halo; and R⁵ is H, C₁₋₆ alkyl, deuterated C₁₋₆ alkyl, C₁₋₆ alkynyl, cyano, C₁₋₆ haloalkyl, C₁₋₆ alkoxy, C₁₋₆ haloalkoxy, SO₂ C₁₋₆ alkyl, SC₁₋₆ alkyl, halo or C₃₋₈ cycloalkyl and the remainder of the variables are as defined in the the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh or twenty-eighth embodiment.

In a thirtieth embodiment, the invention provides a compound represented by structural formula (I′), (I), (II), (IIa), (IIb), (IIc), (III), (IV) (V) or (VI), or a pharmaceutically acceptable salt thereof, wherein R^{2b} is C₁₋₆ alkyl, halo, C₁₋₆ alkoxy, (CH₂)ₙOR⁷ or 4-6 membered heterocycle; R⁴ is H or halo and R⁵ is H, C₁₋₆ alkyl, cyano, C₁₋₆ haloalkyl, halo or C₃₋₈ cycloalkyl and the remainder of the variables are as defined in the second, fourth, sixth, eighth, tenth, eleventh, twelfth, thirteenth, fifteenth, sixteenth, seventeenth, eighteenth, nineteenth, twentieth, twenty-first, twenty-second, twenty-third, twenty-fourth, twenty-fifth, twenty-sixth or twenty-eighth embodiment.

In a thirty-first embodiment, the invention provides a compound represented by structural formula (I), (II), (IIa), (IIb), (IIc), (III), (IV) (V) or (VI), or a pharmaceutically acceptable salt thereof, wherein R^{2b} is H, methyl, ethyl, chloro, OCH₃, CH₂OCH₃ or oxetane, R⁴ is H, OCH₃ or fluoro, R⁵ is H, fluoro, chloro, bromo, iodo, cyano, OCH₃, SCH₃, SO₂CH₃, CHF₂, CF₃, methyl, ethyl, iso-propropyl, iso-butyl, CD₃, C≡CH, OCF₃, OCHF₂ or cyclopropyl or two R⁵ groups on adjacent phenyl ring atoms form OCH₂CH₂O; and R⁶ is H or methyl and the remainder of the variables are as defined in the the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth or thirtieth embodiment.

In a thirty-second embodiment, the invention provides a compound represented by structural formula (I), (II), (IIa), (IIb), (IIc), (III), (IV) (V) or (VI), or a pharmaceutically acceptable salt thereof, wherein R^{2b} is methyl, chloro, OMe, CH₂OCH₃ or oxetane, R⁴ is H or fluoro, R⁵ is H, fluoro, chloro, bromo, cyano, CF₃, methyl, ethyl, or cyclopropyl and R⁶ is H or methyl and the remainder of the variables are as defined in the the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth or thirty-first embodiment.

In a thirty-third embodiment, the invention provides a compound represented by structural formula (I′), (I), (II), (IIa), (IIb), (IIc), (III), (IV) (V) or (VI), or a pharmaceutically acceptable salt thereof, wherein R⁹ is OCH₃, methyl, or NHCH₃ and R¹⁰ is H, methyl, ethyl or propyl and the remainder of the variables are as defined in the the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third, twenty-fourth, twenty-fifth, twenty-sixth, twenty-seventh, twenty-eighth, twenty-ninth, thirtieth, thirty-first or thirty-second embodiment.

In a thirty-fourth embodiment, the invention provides a compound represented by structural formula (I), (II), (IIa), (IIb), (IIc), (III), (IV) (V) or (VI), or a pharmaceutically acceptable salt thereof, wherein R⁷ is H or methyl, R⁹ is OCH₃, methyl, or NHCH₃ and R¹⁰ is methyl and the remainder of the variables are as defined in the second, fourth, sixth, eighth, tenth, eleventh, twelfth, thirteenth, fifteenth, sixteenth, seventeenth, eighteenth, nineteenth, twentieth, twenty-first, twenty-second, twenty-third, twenty-fourth, twenty-fifth, twenty-sixth, twenty-eighth, thirtieth or thirty-second embodiment.

In some embodiments, the present disclosure provides a compound according to structural formula (I), (II), (IIa), (IIb), (IIc), (III), (IV) (V) or (VI), or any one of the compounds disclosed in the examples (including intermediates), both neutral forms or a pharmaceutically acceptable salt thereof.

In some embodiments, the present disclosure provides a compound according to structural formula (I), (II), (IIa), (IIb), (IIc), (III), (IV) (V) or (VI), or any one of the compounds disclosed in the examples (including intermediates), or a pharmaceutically acceptable salt thereof, wherein one or more hydrogen is replaced with deuterium.

In the compounds disclosed herein, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound disclosed herein having deuterium enrichment at one or more positions, e.g., a deuterium enrichment of at least, 50, 80, 90, 95, 98 or 99%.

### Definitions

The term “pharmaceutically-acceptable salt” refers to a salt that is, within the scope of sound medical judgment, suitable for use in contact with the tissues of humans and lower animals without undue toxicity, irritation, and allergic response, and is commensurate with a reasonable benefit/risk ratio. Pharmaceutically-acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

The term “halo” as used herein means halogen and includes chloro, fluoro, bromo and iodo.

The term “alkyl” used alone or as part of a larger moiety, such as “alkoxy” or “haloalkyl” and the like, means saturated aliphatic straight-chain or branched monovalent hydrocarbon radical. Unless otherwise specified, an alkyl group has one to six carbon atoms, i.e. (C₁-C₆)alkyl. Examples include methyl, ethyl, n-propyl, iso-propyl, iso-butyl, and the like.

The term “alkenyl” refers to an unsaturated hydrocarbon group which may be linear or branched and has at least one carbon-carbon double bond. Unless otherwise specified, an alkenyl group has from 2-6 carbon atoms Examples of alkenyl groups include ethenyl, n-propenyl, isopropenyl, n-but-2-enyl, n-pentenyl, n-hex-3-enyl and the like.

The term “alkynyl” refers to an unsaturated hydrocarbon group which may be linear or branched and has at least one carbon-carbon triple bond. Unless specified otherwise, alkynyl groups have from 2-6 carbon atoms. Examples of alkynyl groups include ethynyl, n-propynyl, n-but-2-ynyl, n-hex-3-ynyl and the like.

The term “alkoxy” means an alkyl radical attached through an oxygen linking atom, represented by —O-alkyl. For example, “(C₁-C₆)alkoxy” includes methoxy, ethoxy, propoxy, and butoxy.

The terms “haloalkyl” means alkyl, substituted with one or more halogen atoms.

The term “cycloalkyl” refers to a monocyclic saturated hydrocarbon ring system. Unless otherwise specified, cycloalkyl has from 3-8 carbon atoms. For example, a C₃-C₈ cycloalkyl includes cyclopropyl, cyclobutyl, cyclopentyl, cyclohexyl, cycloheptyl and cyclooctyl.

The term “heteroaryl”, refers to monocyclic aromatic ring groups having five or six ring atoms (i.e., “5-6 membered”) selected from carbon and at least one (typically 1 to 4, more typically 1 or 2) heteroatoms (e.g., oxygen, nitrogen, nitric oxide, sulfur, sulfur oxide or sulfur dioxide). Alternatively, the term “heteroaryl”, refers to bicyclic aromatic ring groups having eight to ten ring atoms (i.e., “8-10 membered”) selected from carbon and at least one (typically 1 to 4, more typically 1 or 2) heteroatoms (e.g., oxygen, nitrogen, nitric oxide, sulfur, sulfur oxide or sulfur dioxide).

Examples of monocyclic heteroaryl groups include furanyl (e.g., 2-furanyl, 3-furanyl), imidazolyl (e.g., N-imidazolyl, 2-imidazolyl, 4-imidazolyl, 5-imidazolyl), isoxazolyl (e.g., 3-isoxazolyl, 4-isoxazolyl, 5-isoxazolyl), oxadiazolyl (e.g., 2-oxadiazolyl, 5-oxadiazolyl), oxazolyl (e.g., 2-oxazolyl, 4-oxazolyl, 5-oxazolyl), pyrazolyl (e.g., 3-pyrazolyl, 4-pyrazolyl), pyrrolyl (e.g., 1-pyrrolyl, 2-pyrrolyl, 3-pyrrolyl), pyridyl (e.g., 2-pyridyl, 3-pyridyl, 4-pyridyl), pyrimidinyl (e.g., 2-pyrimidinyl, 4-pyrimidinyl, 5-pyrimidinyl), pyridazinyl (e.g., 3-pyridazinyl), thiazolyl (e.g., 2-thiazolyl, 4-thiazolyl, 5-thiazolyl), triazolyl (e.g., 2-triazolyl, 5-triazolyl), thiadiazolyl (e.g., 1,2,4-thiadiazolyl, 1,3,4-thiadiazolyl), tetrazolyl (e.g., tetrazolyl), thienyl (e.g., 2-thienyl, 3-thienyl), pyrimidinyl, pyridinyl and pyridazinyl.

Examples of 8- to 10-membered bicyclic heteroaryls include, but are not limited to pyrazolopyridyl, indolyl, indazolyl, azaindolyl, benzimidazolyl, benzoxazolyl, benzothiazolyl, benzofuranyl, benzothiofuranyl, quinolinyl, isoquinolinyl and the like.

The term “heterocyclyl” or “heterocycle” refers to a monocyclic non-aromatic ring radical containing from 3-7 ring atoms (i.e., “3-7 membered”) selected from carbon atom and 1 or 2 heteroatoms. Each heteroatom is independently selected from nitrogen, quaternary nitrogen, oxidized nitrogen (e.g., NO); oxygen; and sulfur, including sulfoxide and sulfone. Representative heterocyclyl groups include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like.

The number of carbon atoms in a group is specified herein by the prefix “Cₓ₋ₓₓ”, wherein x and xx are integers. For example, “C₁₋₆ alkyl” is an alkyl group which has from 1 to 6 carbon atoms.

Certain moieties (e.g., alkyl or cycloalkyl) are referred to herein as being either “substituted” or “optionally substituted”. When a moiety is modified by one of these terms, unless otherwise noted, it denotes that any portion of the moiety that is known to one skilled in the art as being available for substitution can be substituted. If more than one substituent is present, then each substituent may be independently selected. Such means for substitution are well-known in the art and/or taught by the instant disclosure.

### Pharmaceutical Compositions

The compounds disclosed herein are mitogen-activated protein kinase (MEK) inhibitors. The pharmaceutical composition of the present invention comprises one or more MEK inhibitors, or a pharmaceutically acceptable salt thereof, and a pharmaceutically acceptable carrier or diluent.

“Pharmaceutically acceptable carrier” and “pharmaceutically acceptable diluent” refer to a substance that aids the formulation and/or administration of an active agent to and/or absorption by a subject and can be included in the compositions of the present disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds.

The pharmaceutical compositions of the present invention optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

In certain embodiments, the invention provides methods of inhibiting mitogen-activated protein kinase (MEK) or extra cellular signal-regulated kinases (ERK) in a subject in need thereof, comprising: administering to the subject an effective amount of the compounds of the invention, or a pharmaceutically acceptable salt thereof, or an effective amount of the pharmaceutical composition thereof.

A “subject” is a mammal in need of treatment. The mammal can be a veterinary animal (e.g., dog or cat, and the like), farm animal (e.g., horse, cow, sheep or goat and the like) or laboratory animal (e.g., mouse, rat or guinea pig and the like). Most commonly, the subject is a human.

A “subject in need of treatment” is a subject with a disease in which medical treatment is desirable. In some embodiments, the disease is cancer. In some embodiments, the cancer is selected from the group consisting of breast cancer, prostate cancer, esophageal cancer, colon cancer, endometrial cancer, blood cancer, brain cancer, glioma, head and neck cancer, thyroid cancer, gallbladder cancer, bladder cancer, skin cancer, malignant melanoma, cancer of the uterus, cancer of the ovary, lung cancer, pancreatic cancer, liver cancer, renal cancer, testicular cancer, renal pelvic and ureteral cancer, prostate cancer, gastric cancer, stomach cancer, and hematological cancer.

In some embodiments, the lung cancer is selected from the group consisting of non-small cell lung cancer, small cell lung cancer, and lung carcinoid tumor.

In some embodiments, the head and neck cancer is selected from the group consisting of pharyngeal cancer, laryngeal cancer, tongue cancer, and the like.

In some embodiments, the hematological cancer is selected from the group consisting of leukemia, lymphoma, and multiple myeloma.

In some embodiments, the hematological cancer is acute myeloblastic leukemia, chronic myeloid leukemia, B cell lymphoma, chronic lymphocytic leukemia (CLL), Non-Hodgkins lymphoma, hairy cell leukemia, Mantle cell lymphoma, Burkitt lymphoma, small lymphocytic lymphoma, follicular lymphoma, lymphoplasmacytic lymphoma, extranodal marginal zone lymphoma, activated B-cell like (ABC) diffuse large B cell lymphoma, or germinal center B cell (GCB) diffuse large B cell lymphoma.

In some embodiments, the leukemia is selected from the group consisting of acute lymphoblastic leukemia (ALL), chronic lymphocytic leukemia (CLL), acute myelogenous leukemia (AML), acute myelocytic leukemia, acute lymphocytic leukemia, chronic myeloid leukemia (CML), chronic myelocytic leukemia, chronic lymphocytic leukemia, hairy cell leukemia, T-cell prolymphocytic leukemia, juvenile myelomonocytic leukemia, myelodysplastic syndrome, and follicular lymphoma.

In some embodiments, the lymphoma is Hodgkin's lymphoma or non-Hodgkin's lymphoma (NHL).

In some embodiments, the non-Hodgkin lymphoma (NHL) is selected from relapsed NHL, refractory NHL, and recurrent follicular NHL.

In some embodiments, the methods comprise administering an effective amount of a compound disclosed herein, or a pharmaceutically acceptable salt thereof, in combination with an effective amount of an anticancer agent, wherein the amounts of the combination and the chemotherapeutic are together effective in treating a subject with cancer. Many chemotherapeutics are presently known in the art and can be used in combination. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens. Also described are methods for treating a subject with cancer comprising administering to the mammal an amount of a MEK protein kinase inhibitor and/or Raf protein kinase inhibitor in combination with radiation therapy, wherein the amounts of the MEK protein kinase inhibitor and/or Raf protein kinase inhibitor in combination with the radiation therapy effective in treating a subject with cancer. Techniques for administering radiation therapy are known in the art, and these techniques can be used in the combination therapy described herein.

In some embodiments, the disclosure also relates to a method of inhibiting abnormal cell growth in a mammal which may comprises a compound disclosed herein, or a pharmaceutically acceptable salt thereof, and an amount of one or more substances selected from anti-angiogenesis agents, signal transduction inhibitors, and antiproliferative agents. Anti-angiogenesis agents, such as MMP-2 (matrix-metalloprotienase 2) inhibitors, MMP-9 (matrix-metalloprotienase 9) inhibitors, and COX-11 (cyclooxygenase 11) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931, 788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 19911), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997). Some MMP-2 and MMP-9 inhibitors have little or no activity inhibiting MMP-1, while some selectively inhibit MMP-2 and/or AMP-9 relative to the other matrix-motalloproteinases (L e., MAP-1, NEMP-3, MMP-4, M7vlP-5, MMP-6, MMP-7, MMP-8, MMP-10, MMP-11, and MMP-13). Some specific examples of MlvlP inhibitors useful in the present invention are AG-3340, RU 32-3555, and RS 13-0830.

In some embodiments, a compound disclosed herein or a pharmaceutically acceptable salt thereof, is administered with at least one additional therapeutic agent. In some embodiments, the therapeutic agent is a taxol, bortezornib or both. In further or additional embodiments, the therapeutic agent is selected from the group consisting of cytotoxic agents, anti-angiogenesis agents and anti neoplastic agents. In further or additional embodiments, the anti-neoplastic agents selected from the group of consisting of alkylating agents, anti-metabolites, epiclophyllotoxims; antineoplastic enzymes, topoisomerase inhibitors, procarbazine, mitoxantrone, platinum coordination complexes, biological response modifiers and growth inhibitors, hormonal/anti-hormonal therapeutic agents, and haematopoietic growth factors.

Many chemotherapeutics are presently known in the art and can be used in combination with the compounds and compositions of the disclosure. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

In some embodiments, the combination is administered in combination with an additional therapy. In further or additional embodiments, the additional therapy is radiation therapy, chemotherapy, surgery or any combination thereof. In further or additional embodiments, the combination is administered in combination with at least one additional therapeutic agent. In further or additional embodiments, the therapeutic agent is selected from the group of cytotoxic agents, anti-angiogenesis agents and anti-neopiastic agents. In further or additional embodiments, the anti-neoplastic agent is selected from the group of consisting of alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, and haematopoietic growth factors.

In some embodiments, the second therapeutic is an agent for co-regulating MEK or RAF pathways. In some embodiments, the second therapeutic agent is a MEK or RAF inhibitor. In some embodiments, the RAF inhibitor is vemurafenib, dabrafenlb, XL-281, LGX-818, CEP-32496. ARQ-736, MEK-162, Sdumdinib, refametinib, E-620L pimasertib, WX-554, GDC-0973 or LXH254.

In some embodiments, the second therapeutic is an agent for co-regulating MAPK pathway. In some embodiments, the agent for co-regulating MAPK pathway is KRAS G12C mutant selective inhibitors including but not limited to sotorasib adagrasib, ARS-1620, ARS-3248, LY3499446, AMG-510, and MRTX849; KRAS G12D mutant selective inhibitors; Son of Sevenless 1 (SOS1) inhibitors (e.g., BI1701963, BI-3406 and RMC-023); SHP2 inhibitors (e.g, TNO155, BBP-398 and ICP-189); EGFR inhibitors including but not limited to gefitinib, erlotinib, afatinib, lazertinib, aumolertinib (formerly almonertinib), olmutinib, dacomitinib, nazartinib and osimertinib.

In some embodiments, the second therapeutic is an agent for mutant p53 reactivators (PC14586, APR-246 and COTI-2).

In some embodiments, the second therapeutic agent is selected from aspirin; diflunisal; salsalate; acetaminophen; ibuprofen; dexibuprofen; naproxen; fenoprofen; ketoprofen; dexketoprofen; flurbiprofen; oxaprozin; loxoprofen; indomethacin; tolmetin; sulindac; etodolac; ketorolac; diclofenac; aceclofenac; nabumetone; enolic acid; piroxicam; meloxicam; tenoxicam; droxicam; lomoxicam; isoxicam; mefenamic acid; meclofenamic acid; flufenamic acid; tolfenamic acid; sulfonanilides; clonixin; licofelone; dexamethasone; and prednisone.

In some embodiments, the second therapeutic agent is selected from mechlorethamine; cyclophosphamide; melphalan; chlorambucil; ifosfamide; busulfan; N-nitroso-N-methylurea (MNU); carmustine (BCNU); lomustine (CCNU); semustine (MeCCNU); fotemustine; streptozotocin; dacarbazine; mitozolomide; temozolomide; thiotepa; mytomycin; diaziquone (AZQ); cisplatin; carboplatin; and oxaliplatin.

In some embodiments, the second therapeutic agent is selected from vincristine; vinblastine; vinorelbine; vindesine; vinflunine; paclitaxel; docetaxel; etoposide; teniposide; tofacitinib; ixabepilone; irinotecan; topotecan; camptothecin; doxorubicin; mitoxantrone; and teniposide.

In some embodiments, the second therapeutic agent is selected from actinomycin; bleomycin; plicamycin; mitomycin; daunombicin; epimbicin; idarubicin; pirarubicin; aclarubicin; mitoxantrone; cyclophosphamide; methotrexate; 5-fluorouracil; prednisolone; folinic acid; methotrexate; melphalan; capecitabine; mechlorethamine; uramustine; melphalan; chlorambucil; ifosfamide; bendamustine; 6-mercaptopurine; and procarbazine.

In some embodiments, the second therapeutic agent is selected from cladribine; pemetrexed; fludarabine; gemcitabine; hydroxyurea; nelarabine; cladribine; clofarabine; ytarabine; decitabine; cytarabine; cytarabine liposomal; pralatrexate; floxuridine; fludarabine; colchicine; thioguanine; cabazitaxel; larotaxel; ortataxel; tesetaxel; aminopterin; pemetrexed; pralatrexate; raltitrexed; pemetrexed; carmofur; and floxuridine.

In some embodiments, the second therapeutic agent is selected from azacitidine; decitabine; hydroxycarbamide; topotecan; irinotecan; belotecan; teniposide; aclarubicin; epimbicin; idarubicin; amrubicin; pirarubicin; valrubicin; zombicin; mitoxantrone; pixantrone; mechlorethamine; chlorambucil; prednimu stine; uramustine; estramustine; carmustine; lomustine; fotemustine; nimustine; ranimustine; carboquone; thioTEPA; triaziquone; and triethylenemelamine.

In some embodiments, the second therapeutic agent is selected from nedaplatin; satraplatin; procarbazine; dacarbazine; temozolomide; altretamine; mitobronitol; pipobroman; actinomycin; bleomycin; plicamycin; aminolevulinic acid; methyl aminolevulinate; efaproxiral; talaporfin; temoporfin; verteporfin; alvocidib; seliciclib; palbociclib; bortezomib; carfilzomib; anagrelide; masoprocol; olaparib; belinostat; panobinostat; romidepsin; vorinosta; idelalisib; atrasentan; bexarotene; testolactone; amsacrine; trabectedin; alitretinoin; tretinoin; demecolcine; elsamitrucin; etoglucid; lonidamine; lucanthone; mitoguazone; mitotane; oblimersen; omacetaxine mepesuccinate; and eribulin.

In some embodiments, the second therapeutic agent is selected from azathioprine; Mycophenolic acid; leflunomide; teriflunomide; tacrolimus; cyclosporin; pimecrolimus; abetimus; gusperimus; lenalidomide; pomalidomide; thalidomide; anakinra; sirolimus; everolimus; ridaforolimus; temsirolimus; umirolimus; zotarolimus; eculizumab; adalimumab; afelimomab; certolizumab pegol; golimumab; infliximab; nerelimomab; mepolizumab; omalizumab; faralimomab; elsilimomab; lebrikizumab; ustekinumab; etanercept; otelixizumab; teplizumab; visilizumab; clenoliximab; keliximab; zanolimumab; efalizumab; erlizumab; obinutuzumab; rituximab; and ocrelizumab.

In some embodiments, the second therapeutic agent is selected from pascolizumab; gomiliximab; lumiliximab; teneliximab; toralizumab; aselizumab; galiximab; gavilimomab; ruplizumab; belimumab; blisibimod; ipilimumab; tremelimumab; bertilimumab; lerdelimumab; metelimumab; natalizumab; tocilizumab; odulimomab; basiliximab; daclizumab; inolimomab; zolimoma; atorolimumab; cedelizumab; fontolizumab; maslimomab; morolimumab; pexelizumab; reslizumab; rovelizumab; siplizumab; talizumab; telimomab; vapaliximab; vepalimomab; abatacept; belatacept; pegsunercept; aflibercept; alefacept; and rilonacept.

In some embodiments, the second therapeutics is an immune checkpoint inhibitor such as a PD-1 inhibitoror a PD-L1 inhibitor. In some embodiments, the immune checkpoint inhibitor is an anti PD-1 antibody selected from the group consisting of balstilimab, camrelizumab, cemiplimab, dostarlimab, geptanolimab, nivolumab, pembrolizumab, penpulimab, pidilizumab, prolgolimab, retifanlimab, sasanlimab, serplulimab, serplulimab, sintilimab, spartalizumab, sulituzumab, tebotelimab, teripalimab, tislelizumab, toripalimab, toripalimab, zimberelimab, AMP-224 (Medlmunne), AMP-514 (Medlmunne), AT-16201 (AIMM Therapeutics BV), AVI-102 (Ab Vision Inc), BAT-1308 (Bio-Thera Solutions Ltd), BH-2950 (Beijing Hanmi Pharmaceutical Co Ltd), BSI-050K01 (Biosion Inc), CB-201 (Crescendo Biologies Ltd), CYTO-101 (Cytocom Inc), DB-004 (DotBio Pte Ltd), EX-105 (Excelmab Inc), EX-108 (Excelmab Inc), GNR-051 (Generium), HAB-21 (Suzhou Stainwei Biotech Inc), IBI-319 (Innovent Biologies Inc), IBI-321 (Innovent Biologies Inc), IKT-202 (Icell Kealex Therapeutics LLC), IMU-201 (Imugene Ltd), JS-201 (Shanghai Junshi Bioscience Co Ltd), LBL-006 (Leads Biolabs Inc), LBL-024 (Leads Biolabs Inc), LD-01 (Leidos Health Holdings LLC), LQ-005 (Shanghai Novamab Biopharmaceuticals Co Ltd), LQ-008 (Shanghai Novamab Biopharmaceuticals Co Ltd), MD-402 (MD Biosciences GmbH), OT-2 (OncoTrap Inc), PE-0105 (Shanghai Yunyi Health Technology Development Co Ltd), PF-07209960 (Pfizer Inc), PH-762 (Phio Pharmaceuticals Corp), REGN-PD-1/XX (Regeneron), R07121661 (Genentech), SAUG-1 (Juvenescence UK Ltd), SCT-IIOA (Sinocelltech), SG-001 (CSPC Pharmaceutical Group Ltd), SI-B003 (SystImmune), SL-279137 (Shattuck Labs), SSI-361 (Lyvgen Biopharma Ltd), STI-A1110 (Servier), STM-418 (Stcube Inc), Sym-021 (Symphogen A/S), TSR-075 (GlaxoSmithKline Pic), TY101 (Tayu Huaxia Biotech), Twist-PD-1 (Twist Bioscience), XmAb-TGFpR2 (Xencor), XmAb-YYCD28 (Xencor), XmAb20717 (Xencor), XmAb23104 (Xencor), YBL-006 (Y Biologies), YBL-019 (Y Biologies), and mDX-400 (Merck & Co Inc).

In one embodiment, the anti-cancer agent and the compound represented by structural formula (I) are administered contemporaneously. When administered contemporaneously, the anti-cancer agent and the compound can be administered in the same formulation or in different formulations. Alternatively, the compound and the additional anti-cancer agent are administered separately. Alternatively, the compound and the additional anti-cancer agent can be administered sequentially, as separate compositions, within an appropriate time frame (e.g., a cancer treatment session/interval (e.g., about 1.5 to about 5 hours to about 10 hours to about 15 hours to about 20 hours; about 1 day to about 2 days to about 5 days to about 10 days to about 14 days)) as determined by the skilled clinician (e.g., a time sufficient to allow an overlap of the pharmaceutical effects of the therapies). The compound and the additional anti-cancer agent can be administered in a single dose or multiple doses in an order and on a schedule suitable to achieve a desired therapeutic effect (e.g., inhibition of tumor growth).

Thus the present invention provides a method of treatment comprising administering to a subject a compound represented by structural formula (I) or a pharmaceutically acceptable salt thereof so as to treat at least one of the diseases or conditions listed above.

As used herein, the term “treating” or ‘treatment” refers to obtaining a desired pharmacological and/or physiological effect. The effect can be therapeutic, which includes achieving, partially or substantially, one or more of the following results: partially or totally reducing the extent of the disease, disorder or syndrome; ameliorating or improving a clinical symptom or indicator associated with the disorder; or delaying, inhibiting or decreasing the likelihood of the progression of the disease, disorder or syndrome.

### Methods of Administration and Dosage Forms

The precise amount of compound administered to provide an “effective amount” to the subject will depend on the mode of administration, the type, and severity of the disease or condition, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of the invention being used by following, for example, dosages reported in the literature and recommended in the Physician's Desk Reference (57th Ed., 2003).

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed MEK inhibitors for guidance.

The compounds or the corresponding pharmaceutical compositions taught herein can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the invention is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the present teachings may be incorporated with excipient and used in the form of ingestible tablets, buccal tablets, troches, capsules, elixirs, suspensions, syrups, wafers, and the like.

Typically for parenteral administration, solutions of a compound of the present teachings can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound described herein for the extemporaneous preparation of sterile injectable solutions or dispersions are appropriate.

The following Examples are offered to illustrate exemplary embodiments of the invention and do not define or limit its scope.

### EXEMPLIFICATION

The abbreviations used in the entire specification may be summarized herein below with their particular meaning:
ACN, MeCN—Acetonitrile;
AcOK—Potassium acetate;
AIBN—Azobisisobutyronitrile
BOC—tert-Butyloxycarbonyl;
Boc₂O—Di-tert-butyl dicarbonate;
° C.—degree Celsius;
CaCl₂—Calcium chloride;
CBr₄—Carbon tetrabromide;
CDCl₃ or Chloroform-d—Deuterated chloroform;
Cs₂CO₃—Cesium carbonate;
d—Doublet;
δ—Delta;
DCM, CH₂Cl₂—Dichloromethane;
DMA—N, N-Dimethylacetamide;
DMAP—4-Dimethylaminopyridine;
DMF—N, N-Dimethylformamide;
DMSO—Dimethylsulfoxide;
DMSO-d₆—Deuterated dimethylsulfoxide;
ESI—Electrospray ionization;
EtOAc, EA—Ethyl acetate;
Et₃N—Triethylamine;
FA—Formic acid;
¹⁹F NMR—Fluorine-19 nuclear magnetic resonance;
g—Gram;
h—Hour;
¹H—Proton;
¹H NMR—Proton nuclear magnetic resonance;
H₂O—Water;
HCl—Hydrochloric acid;
HP—High performance;
Hz—Hertz;
J—Coupling constant;
K₂CO₃—Potassium carbonate;
LCMS—Liquid chromatography mass spectrometry;
M⁺—Molecular ion;
m—Multiplet;
MeOH—Methanol;
Methanol-d₄—Deuterated methanol;
mg—Milligrams;
min—Minutes;
MHz—Mega Hertz (frequency);
mL—Milliters;
mmol—Millimoles;
MnO₂—Manganese dioxide;
NaBH₄—Sodium borohydride;
NaHCO₃—Sodium hydrogencarbonate;
Na₂SO₄—Sodium sulfate;
NBS—N-Bromosuccinimide;
NH₃H₂O—Ammonia aqueous;
NH₄Cl—Ammonium chloride;
NH₄HCO₃—Ammonium bicarbonate
Pd(dppf)Cl₂—[1,1′-Bis(diphenylphosphino)ferrocene]dichloropalladium(II);
Pd₂(dba)₃—Tris(dibenzylideneacetone)dipalladium(0);
PE—Petroleum ether;
%—Percentage;
pH—potential of Hydrogen;
PPh₃—Triphenylphosphine;
ppm—Parts per million;
q—Quartet;
RuPhos—2-Dicyclohexylphosphino-2′,6′-diisopropoxybiphenyl;
RuPhos Pd G₃—(2-Dicyclohexylphosphino-2′,6′-diisopropoxy-1,1′-biphenyl)[2-(2′-amino-1,1′-biphenyl)]palladium(II) methanesulfonate;
s—Singlet;
t—Triplet;
TBAF—Tetrabutylammonium fluoride;
TFA—Trifluoroacetic acid;
THF—Tetrahydrofuran;
TMSCHN₂—(Trimethylsilyl)diazomethane;
XantPhos—4,5-Bis(diphenylphosphino)-9,9-dimethylxanthene.

### Intermediate 1

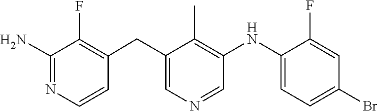 [SMILES: Cc1c(Cc2ccnc(N)c2F)cncc1Nc1ccc(Br)cc1F]

### Synthetic Route

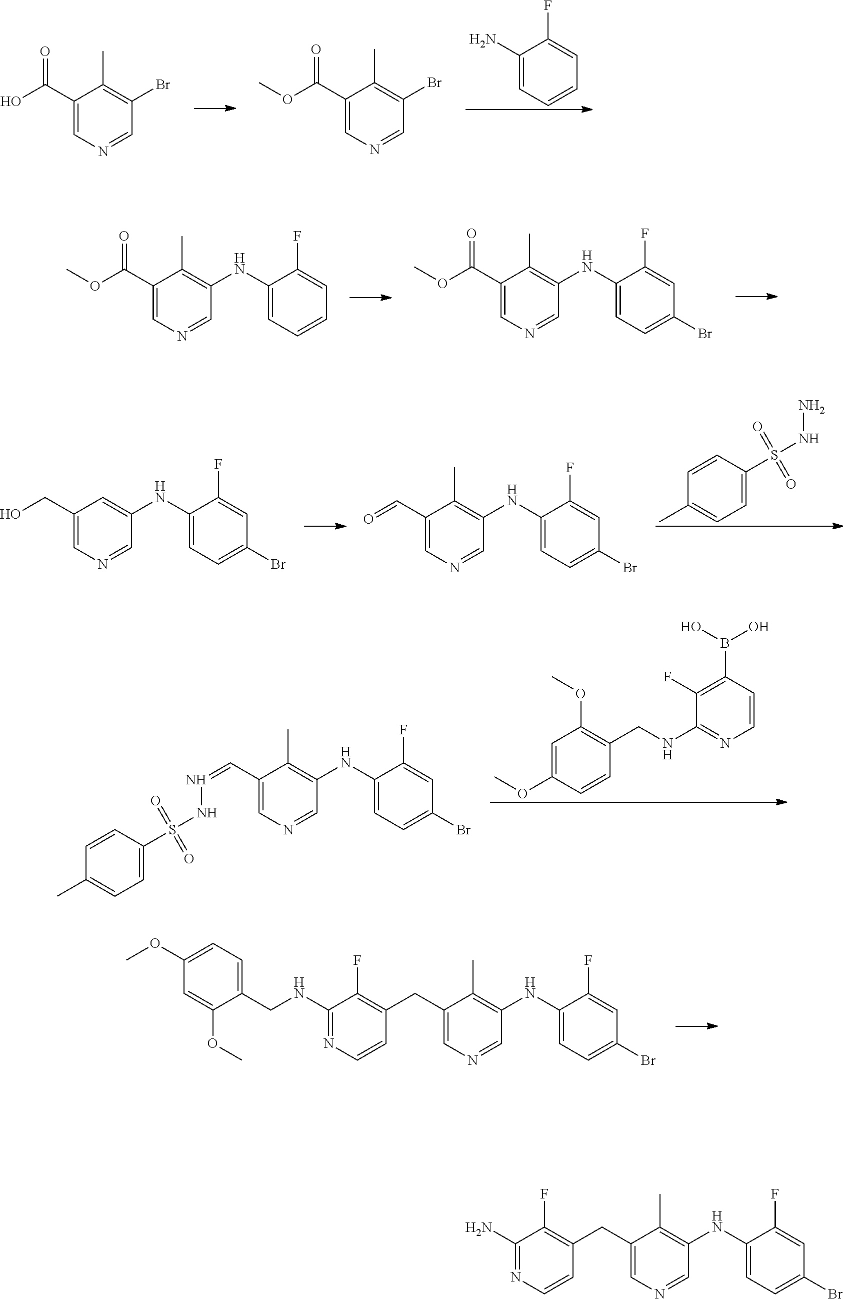

### Synthesis of Intermediate 1: Step 1

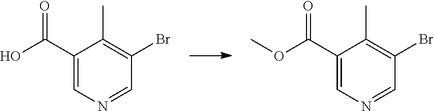

To a stirred solution of 5-bromo-4-methylpyridine-3-carboxylic acid (5 g, 23.2 mmol, 1 equiv) and MeOH (50 mL) in DCM (50 mL) was added TMSCHN₂ (2 M in n-hexane, 23.2 mL, 46.3 mmol, 2.0 equiv) dropwise at 0° C. under nitrogen atmosphere. The resulting mixture was stirred for 6 h at room temperature under nitrogen atmosphere. Desired product could be detected by LCMS. The reaction mixture was concentrated under reduced pressure. The residue was purified by silica gel column chromatography, eluted with PE/EA (5:1) to afford methyl 5-bromo-4-methylpyridine-3-carboxylate (5.0 g, 93%) as a white solid. LCMS: [M+1]⁺=230.0; ¹H NMR (400 MHz, Chloroform-d) δ 8.88 (s, 1H), 8.76 (s, 1H), 3.93 (s, 3H), 2.67 (s, 3H).

### Synthesis of Intermediate 1: Step 2

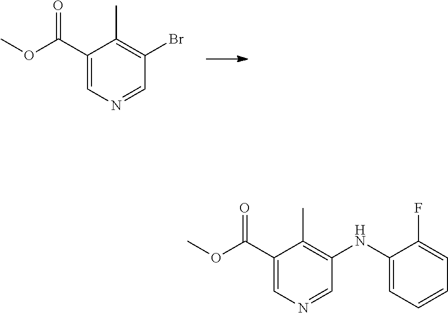 [SMILES: COC(=O)c1cncc(Br)c1C ; COC(=O)c1cncc(Nc2ccccc2F)c1C]

To a stirred mixture of methyl 5-bromo-4-methylpyridine-3-carboxylate (5 g, 21.7 mmol, 1 equiv) and 2-fluoroaniline (4.8 g, 43.5 mmol, 2.0 equiv) in dioxane (50 mL) were added Cs₂CO₃ (21.24 g, 65.19 mmol, 3.0 equiv), RuPhos (1.01 g, 2.2 mmol, 0.1 equiv), and RuPhos Pd G₃ (1.8 g, 2.2 mmol, 0.1 equiv) at room temperature under nitrogen atmosphere. The resulting mixture was stirred for 2 h at 90° C. under nitrogen atmosphere. Desired product could be detected by LCMS. The resulting mixture was concentrated under reduced pressure. The residue was purified by silica gel column chromatography, eluted with PE/EA (1:1) to afford methyl 5-[(2-fluorophenyl)amino]-4-methylpyridine-3-carboxylate (4.5 g, 79%) as a yellow solid. LCMS: (ESI, m/z): [M+1]⁺=261.0; ¹H NMR (400 MHz, Chloroform-d) δ 8.77 (s, 1H), 8.56 (s, 1H), 7.12 (m, 1H), 7.01 (m, 1H), 6.89 (m, 2H), 5.54 (s, 1H), 3.95 (s, 3H), 2.52 (s, 3H).

### Synthesis of Intermediate 1: Step 3

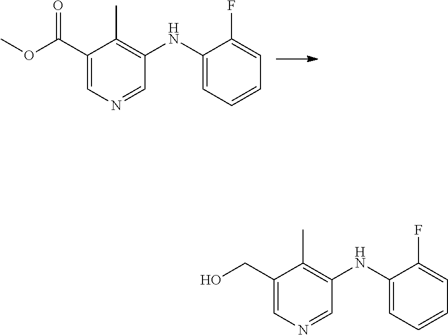 [SMILES: COC(=O)c1cncc(Nc2ccccc2F)c1C ; Cc1c(CO)cncc1Nc1ccccc1F]

To a stirred mixture of methyl 5-[(2-fluorophenyl)amino]-4-methylpyridine-3-carboxylate (2.1 g, 4.8 mmol, 1 equiv, 60% purity) and CaCl₂) (2.7 g, 24.2 mmol, 5 equiv) in MeOH (25 mL) was added NaBH₄ (1.83 g, 48.4 mmol, 10 equiv) in portions at 0° C. under nitrogen atmosphere. The resulting mixture was stirred for 1 h at 0° C. under nitrogen atmosphere. Desired product could be detected by LCMS. The reaction was quenched with Water/Ice at 0° C. The resulting mixture was concentrated under reduced pressure. The residue was purified by silica gel column chromatography, eluted with CH₂Cl₂/MeOH (10:1) to afford {5-[(2-fluorophenyl)amino]-4-methylpyridin-3-yl}methanol (820 mg) as a white solid. LCMS: (ESI, m/z): [M+1]⁺=233.1

### Synthesis of Intermediate 1: Step 4

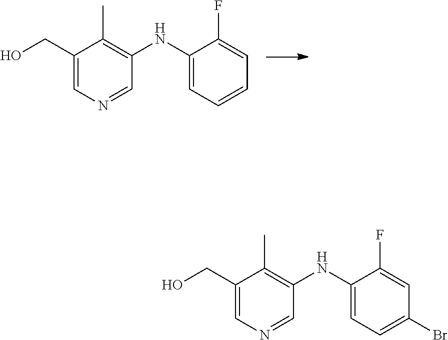 [SMILES: Cc1c(CO)cncc1Nc1ccccc1F ; Cc1c(CO)cncc1Nc1ccc(Br)cc1F]

To a stirred mixture of {5-[(2-fluorophenyl)amino]-4-methylpyridin-3-yl}methanol (100 mg, 0.43 mmol, 1 equiv) in ACN (3 mL) was added NBS (84.3 mg, 0.47 mmol, 1.1 equiv) in portions at −10° C. under nitrogen atmosphere. The resulting mixture was stirred for 2 h at −10° C. under nitrogen atmosphere. Desired product could be detected by LCMS. The resulting mixture was concentrated under reduced pressure. The residue was purified by silica gel column chromatography, eluted with PE/EA (1:1) to afford {5-[(4-bromo-2-fluorophenyl)amino]-4-methylpyridin-3-yl}methanol (80 mg, 60%) as a yellow solid. LCMS: (ESI, m/z): [M+1]⁺=311.0; ¹H NMR (400 MHz, Chloroform-d) δ 8.36 (m, 2H), 7.32-7.26 (m, 1H), 7.16-7.08 (m, 1H), 6.72 (m, 1H), 4.79 (s, 2H), 2.31 (s, 3H).

### Synthesis of Intermediate 1: Step 5

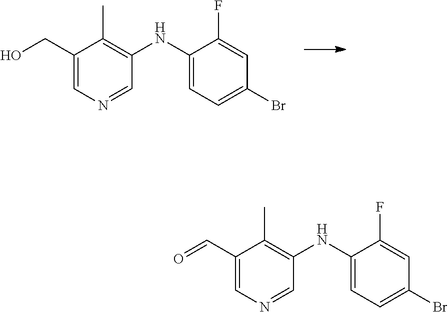 [SMILES: Cc1c(CO)cncc1Nc1ccc(Br)cc1F ; Cc1c(C=O)cncc1Nc1ccc(Br)cc1F]

To a stirred mixture of {5-[(4-bromo-2-fluorophenyl)amino]-4-methylpyridin-3-yl}methanol (780 mg, 2.5 mmol, 1 equiv) in DCM (10 mL) was added MnO₂ (1089.7 mg, 12.5 mmol, 5 equiv) at room temperature under nitrogen atmosphere. The resulting mixture was stirred for 2 h at 50° C. under nitrogen atmosphere. Desired product could be detected by LCMS. The resulting mixture was concentrated under reduced pressure. The residue was purified by silica gel column chromatography, eluted with PE/EA (5:1) to afford 5-[(4-bromo-2-fluorophenyl)amino]-4-methylpyridine-3-carbaldehyde (380 mg, 49%) as a white solid. LCMS: [M+1]⁺=309.0; ¹H NMR (400 MHz, Chloroform-d) δ 10.31 (s, 1H), 8.71 (s, 1H), 8.60 (m, 1H), 7.31 (m, 1H), 7.19-7.13 (m, 1H), 6.74 (m, 1H), 2.60 (s, 3H).

### Synthesis of Intermediate 1: Step 6

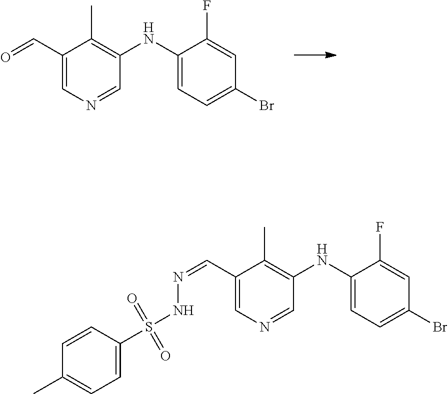 [SMILES: Cc1c(C=O)cncc1Nc1ccc(Br)cc1F ; Cc1ccc(S(=O)(=O)N/N=C\c2cncc(Nc3ccc(Br)cc3F)c2C)cc1]

A mixture of 5-[(4-bromo-2-fluorophenyl)amino]-4-methylpyridine-3-carbaldehyde (400 mg, 1.3 mmol, 1 equiv) and 4-toluenesulfonyl hydrazide (265. mg, 1.4 mmol, 1.1 equiv) in MeOH (8 mL) was stirred for 2 h at room temperature under nitrogen atmosphere. Desired product could be detected by LCMS. The resulting mixture was concentrated under reduced pressure. The residue was purified by silica gel column chromatography, eluted with CH₂Cl₂/MeOH (10:1) to afford N′-[(1Z)-{5-[(4-bromo-2-fluorophenyl)amino]-4-methylpyridin-3-yl}methylidene]-4-methylbenzenesulfonohydrazide (400 mg, crude) as a white solid. LCMS: [M+1]⁺=477.1; ¹H NMR (400 MHz, DMSO-d₆) δ 11.58 (s, 1H), 8.38 (s, 1H), 8.27 (s, 1H), 8.15 (s, 1H), 7.77 (m, 2H), 7.70-7.65 (m, 2H), 7.47 (m, 1H), 7.41 (m, 1H), 7.16 (m, 1H), 6.56 (m, 1H), 2.39 (s, 3H), 2.37 (s, 3H).

### Synthesis of Boronate Step 1

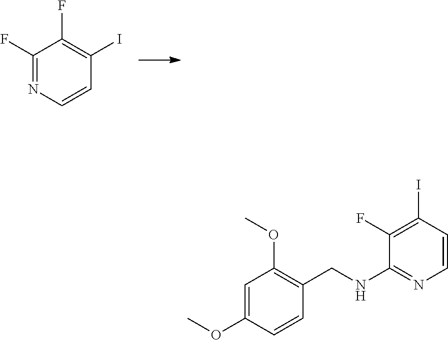 [SMILES: Fc1nccc(I)c1F ; COc1ccc(CNc2nccc(I)c2F)c(OC)c1]

To a stirred solution of 2,3-difluoro-4-iodopyridine (4.8 g, 19.9 mmol, 1 equiv) in NMP (45 mL) were added 1-(2,4-dimethoxyphenyl) methanamine (8.33 g, 49.8 mmol, 2.5 equiv) and Et₃N (6.05 g, 59.8 mmol, 3 equiv) at room temperature under nitrogen atmosphere. The resulting mixture was stirred for additional 1 h at 100° C. Desired product could be detected by LCMS. The mixture was allowed to cool down to room temperature. The resulting mixture was diluted with water (100 mL). The resulting mixture was extracted with EtOAc (3×100 mL). The combined organic layers were washed with water (5×200 mL) and brine (200 mL), dried over anhydrous Na₂SO₄. After filtration, the filtrate was concentrated under reduced pressure. The residue was purified by silica gel column chromatography, eluted with PE/EA (5:1) to afford N-[(2,4-dimethoxyphenyl)methyl]-3-fluoro-4-iodopyridin-2-amine (7 g, 91%) as a brown yellow solid. LCMS: [M+1]⁺=389.0; ¹H NMR (300 MHz, Chloroform-d) δ 7.57 (m, 1H), 7.29 (s, 1H), 6.87 (m, 1H), 6.50 (m, 1H), 6.45 (m, 1H), 5.14 (s, 1H), 4.61 (m, 2H), 3.87 (s, 3H), 3.82 (s, 3H).

### Synthesis of Boronate Step 2

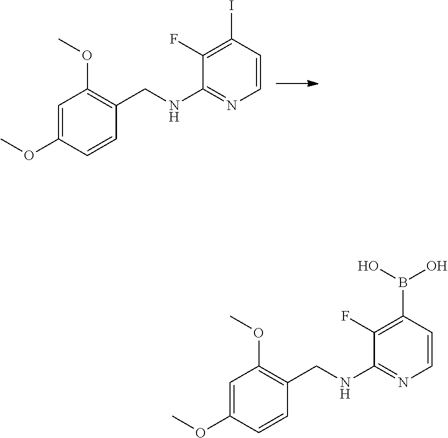 [SMILES: COc1ccc(CNc2nccc(I)c2F)c(OC)c1 ; COc1ccc(CNc2nccc(B(O)O)c2F)c(OC)c1]

To a stirred mixture of N-[(2,4-dimethoxyphenyl)methyl]-3-fluoro-4-iodopyridin-2-amine (2.1 g, 5.4 mmol, 1 equiv) and bis(pinacolato)diboron (2.06 g, 8.1 mmol, 1.5 equiv) in dioxane (20 mL) were added potassium acetate (1.59 g, 16.23 mmol, 3.0 equiv) and Pd(dppf)Cl₂ (0.40 g, 0.541 mmol, 0.1 equiv) at room temperature under nitrogen atmosphere. The resulting mixture was stirred for 16 h at 110° C. under nitrogen atmosphere. Desired product could be detected by LCMS. The resulting mixture was concentrated under reduced pressure. The residue was purified by reverse flash chromatography with the following conditions: column, C18 silica gel; mobile phase, ACN (0.1% FA) in water (0.1% FA), 5% to 30% gradient in 30 min; detector, UV 254/220 nm to afford 2-{[(2,4-dimethoxyphenyl)methyl]amino}-3-fluoropyridin-4-ylboronic acid (940 mg, crude) as a white solid. LCMS: [M+1]+=307.1; ¹H NMR (400 MHz, DMSO-d₆) δ 8.45 (s, 1H), 7.68 (m, 1H), 7.59 (s, 1H), 7.02 (m, 1H), 6.66-6.55 (m, 1H), 6.52 (m, 2H), 6.41 (m, 1H), 4.44 (m, 2H), 3.80 (s, 3H), 3.71 (s, 3H).

### Synthesis of Intermediate 1: Step 7

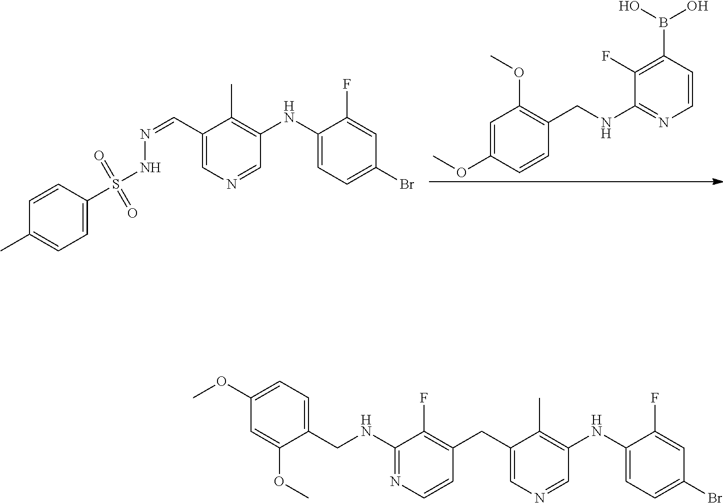

To a stirred mixture of N′-[(1Z)-{5-[(4-bromo-2-fluorophenyl)amino]-4-methylpyridin-3-yl}methylidene]-4-methylbenzenesulfonohydrazide (260 mg, 0.55 mmol, 1 equiv) and 2-{[(2,4-dimethoxyphenyl)methyl]amino}-3-fluoropyridin-4-ylboronic acid (500 mg, 1.635 mmol, 3.0 equiv) in dioxane (8 mL) was added K₂CO₃ (90 mg, 0.65 mmol, 1.2 equiv) at room temperature under nitrogen atmosphere. The resulting mixture was stirred for 2 h at 110° C. under nitrogen atmosphere. Desired product could be detected by LCMS. The resulting mixture was concentrated under reduced pressure. The residue was purified by reverse flash chromatography with the following conditions: column, C18 silica gel; mobile phase, MeCN in water (10 mmol/L NH₄HCO₃), 30% to 60% gradient in 30 min; detector, UV 254/220 nm to afford N-(4-bromo-2-fluorophenyl)-5-[(2-{[(2,4-dimethoxyphenyl)methyl]amino}-3-fluoropyridin-4-yl)methyl]-4-methylpyridin-3-amine (100 mg, 33%) as a white solid. LCMS: [M+1]⁺=555.1; ¹H NMR (400 MHz, Chloroform-d) δ 8.36 (s, 1H), 8.20 (s, 1H), 7.80 (m, 1H), 7.28 (m, 1H), 7.25 (s, 1H), 7.11 (m, 1H), 6.68 (m, 1H), 6.48 (m, 1H), 6.44 (m, 1H), 6.14 (m, 1H), 5.44 (s, 1H), 5.05 (s, 1H), 4.60 (m, 2H), 3.96 (s, 2H), 3.85 (s, 3H), 3.80 (s, 3H), 2.15 (s, 3H).

### Synthesis of Intermediate 1: Step 8

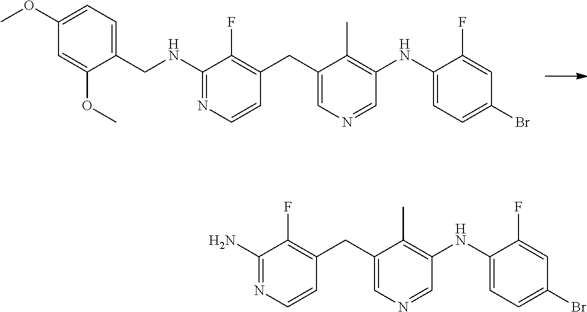 [SMILES: COc1ccc(CNc2nccc(Cc3cncc(Nc4ccc(Br)cc4F)c3C)c2F)c(OC)c1 ; Cc1c(Cc2ccnc(N)c2F)cncc1Nc1ccc(Br)cc1F]

To a stirred mixture of N-(4-bromo-2-fluorophenyl)-5-[(2-{[(2,4-dimethoxyphenyl)methyl]amino}-3-fluoropyridin-4-yl)methyl]-4-methylpyridin-3-amine (25 mg, 0.045 mmol, 1 equiv) in DCM (2 mL) was added TFA (0.5 mL) dropwise at 0° C. under nitrogen atmosphere. The resulting mixture was stirred for 2 h at room temperature under nitrogen atmosphere. Desired product could be detected by LCMS. The resulting mixture was concentrated under reduced pressure. The residue was purified by reverse flash chromatography with the following conditions: column, C18 silica gel; mobile phase, ACN in water (10 mmol/L NH₄HCO₃), 5% to 40% gradient in 30 min; detector, UV 254/220 nm to afford 5-[(2-amino-3-fluoropyridin-4-yl)methyl]-N-(4-bromo-2-fluorophenyl)-4-methylpyridin-3-amine (14 mg, 76%) as a white solid. LCMS: [M+1]⁺=405.0; ¹H NMR (400 MHz, Chloroform-d) δ 8.39 (s, 1H), 8.21 (s, 1H), 7.72 (m, 1H), 7.23 (m, 1H), 7.08 (m, 1H), 6.63 (m, 1H), 6.25 (m, 1H), 5.41 (s, 1H), 4.58 (s, 2H), 3.98 (s, 2H), 2.12 (s, 3H).

### Intermediate 2

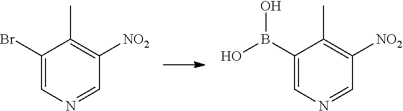

To a solution of 3-bromo-4-methyl-5-nitropyridine (5 g, 23.0 mmol, 1 equiv) and bis(pinacolato)diboron (8.78 g, 34.6 mmol, 1.5 equiv) in dioxane (100 mL) were added AcOK (6.78 g, 69.1 mmol, 3 equiv) and Pd(dppf)Cl₂ (1.69 g, 2.3 mmol, 0.1 equiv). After stirring for 16 h at 110° C. under a nitrogen atmosphere, the resulting mixture was concentrated under reduced pressure. Desired product could be detected by LCMS. The residue was purified by reversed-phase flash chromatography with the following conditions: column, C18 silica gel; mobile phase, MeCN (0.1% FA) in Water (0.1% FA), 10% to 30% gradient in 25 min; detector, UV 254/220 nm. This resulted in 4-methyl-5-nitropyridin-3-ylboronic acid (3.31 g, crude) as a white solid. LCMS: (ESI, m/z): [M+1]⁺=183.1; ¹H NMR (400 MHz, DMSO-d₆) δ 9.06 (s, 1H), 8.80 (s, 1H), 2.62 (s, 3H).

### Intermediate 3

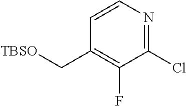 [SMILES: CC(C)(C)[Si](C)(C)OCc1ccnc(Cl)c1F]

### Synthesis of Intermediate 3: Step 1

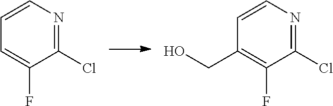 [SMILES: Fc1cccnc1Cl ; OCc1ccnc(Cl)c1F]

To a stirred solution of diisopropyl amine (7.69 g, 76.0 mmol, 1.0 equiv.) in tetrahydrofuran (80 mL) was added butyl lithium (30.5 mL, 2.5 mol/L in hexane) dropwise at −78° C. under nitrogen atmosphere. After stirring for 2 h at −78° C. To the above mixture was added 2-chloro-3-fluoropyridine (10.00 g, 76.0 mmol, 1.0 equiv.) dropwise at −78° C. The resulting mixture was stirred for 2 h at −78° C. DMF (55.6 g, 760.3 mmol, 10.0 equiv.) was added dropwise to the mixture over 30 min at −78° C. The mixture was warmed up to room temperature. After stirring for 2 h, NaBH₄ (3.7 g, 98.8 mmol, 1.3 equiv.) was added to the mixture in portions at 0° C. The resulting mixture was stirred at 0° C. for 1 h. Desired product could be detected by LCMS. The resulting reaction mixture was poured into water, and extracted with EtOAc. The extract was washed with 1.0 M aqueous HCl, saturated aqueous NaHCO₃ and brine. The organic layer was dried over Na₂SO₄ and evaporated. The residue was purified by silica gel column chromatography, eluted with PE/EA (1:1) to afford (2-chloro-3-fluoropyridin-4-yl) methanol (7.8 g, 63%) as a white solid. LCMS: [M+1]+=162.00; ¹H NMR (300 MHz, DMSO-d₆) δ 8.27-8.25 (m, 1H), 7.55 (m, 1H), 5.71 (s, 1H), 4.65 (s, 2H); ¹⁹F NMR (282 MHz, DMSO-d₆) δ −126.65.

### Synthesis of Intermediate 3: Step 2

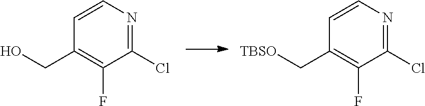 [SMILES: OCc1ccnc(Cl)c1F ; CC(C)(C)[Si](C)(C)OCc1ccnc(Cl)c1F]

To a stirred solution of (2-chloro-3-fluoropyridin-4-yl) methanol (6.8 g, 42.1 mmol, 1.0 equiv.) in DMF (68 mL) were added 1H-Imidazole (8.6 g, 126.3 mmol, 3.0 equiv.) and t-butyl dimethylchlorosilane (12.7 g, 84.2 mmol, 2.0 equiv.) in portions at room temperature under nitrogen atmosphere. Keep stirring for 2 h at room temperature. Desired product could be detected by LCMS. The resulting reaction mixture was diluted with CH₂Cl₂ and washed with brine. The organic layer was dried over Na₂SO₄ and evaporated. The residue was purified by silica gel column chromatography, eluted with CH₂Cl₂ (100%) to afford 4-{[(tert-butyl dimethylsilyl)oxy]methyl}-2-chloro-3-fluoropyridine (11.0 g, 95%) as a white solid. LCMS: [M+1]+=276.10; ¹H NMR (300 MHz, DMSO-d₆) δ 8.29-8.27 (m, 1H), 7.52-7.48 (m, 1H), 4.85 (s, J=1.1 Hz, 2H), 0.91 (s, 9H), 0.11 (s, 6H); ¹⁹F NMR (282 MHz, DMSO-d₆) δ −126.09.

### Intermediate 4

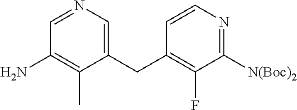

### Synthetic Route

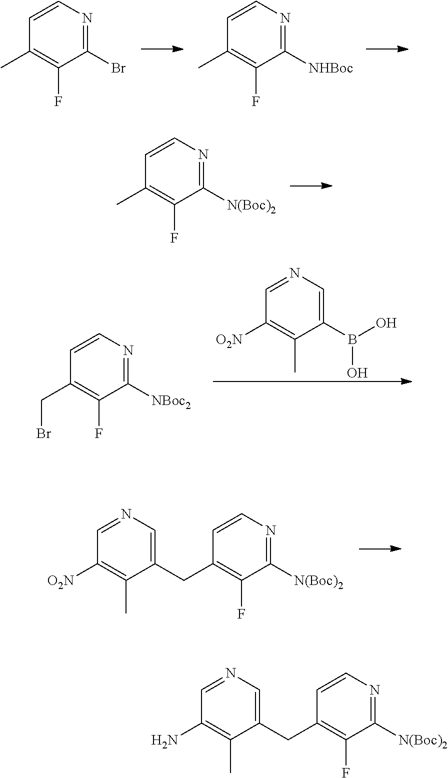

### Synthesis of Intermediate 4: Step 1

Into a 250 mL round bottom flask were added 2-bromo-3-fluoro-4-methylpyridine (10 g, 52.6 mmol, 1 equiv.), BocNH₂ (7.4 g, 63.2 mmol, 1.2 equiv.), Cs₂CO₃ (34.3 g, 105.2 mmol, 2 equiv.), Pd₂(dba)₃ (4.82 g, 5.26 mmol, 0.1 equiv.), XantPhos (3.05 g, 5.26 mmol, 0.1 equiv.) and dioxane (100 mL) at 25° C. under nitrogen atmosphere, and then heated to 80° C., keep stirring for 2 h at 80° C. Desired product could be detected by LCMS. The resulting mixture was concentrated under reduced pressure. The residue was purified by silica gel column chromatography, eluted with PE/EA (1:1) to afford tert-butyl N-(3-fluoro-4-methylpyridin-2-yl) carbamate (2.8 g, 28%) as white solid. LCMS: (ESI, m/z): [M+1]⁺=227.40; ¹H NMR (300 MHz, Chloroform-d) δ 8.07-8.05 (m, 1H), 6.97 (s, 1H), 6.89-6.86 (m, 1H), 2.30 (d, J=1.9 Hz, 3H), 1.53 (s, 9H); ¹⁹F NMR (282 MHz, CDCl₃) δ −137.73.

### Synthesis of Intermediate 4: Step 2

To a stirred solution of tert-butyl N-(3-fluoro-4-methylpyridin-2-yl) carbamate (2.3 g, 10.2 mmol, 1 equiv.) in THF (20 mL) was added Et₃N (2.1 g, 20.3 mmol, 2.0 equiv.) at room temperature, and then (Boc)₂O (2.66 g, 12.19 mmol, 1.2 equiv.) and DMAP (0.12 g, 1.01 mmol, 0.1 equiv.) was added at 0° C., after addition completed, warmed to 25° C., keep stirring at room temperature for 16 h. Desired product could be detected by LCMS. The resulting mixture was concentrated under reduced pressure. The residue was purified by silica gel column chromatography, eluted with PE/EA (1:1) to afford tert-butyl N-(tert-butoxycarbonyl)-N-(3-fluoro-4-methylpyridin-2-yl) carbamate (2.6 g, 65%) as white solid. LCMS: (ESI, m/z): [M+1]⁺=327.20; ¹H NMR (300 MHz, Chloroform-d) δ 8.14 (d, J=4.9 Hz, 1H), 7.16-7.10 (m, 1H), 2.34 (m, 3H), 1.42 (s, 18H); ¹⁹F NMR (282 MHz, CDCl₃) δ −131.69.

### Synthesis of Intermediate 4: Step 3

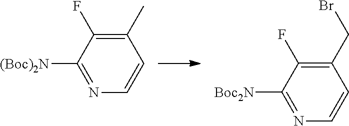

To a stirred solution of tert-butyl N-(tert-butoxycarbonyl)-N-(3-fluoro-4-methylpyridin-2-yl) carbamate (2.6 g, 7.96 mmol, 1.0 equiv.) in DCE (30 mL) were added NBS (4.25 g, 23.89 mmol, 3.0 equiv.) and AIBN (261.64 mg, 1.59 mmol, 0.2 equiv.) at 25° C. under nitrogen atmosphere and then heated to 80° C., keep stirring at 80° C. for 4 h. Desired product could be detected by LCMS. The resulting reaction mixture was concentrated under reduce pressure. The residue was purified by HP flash chromatography with the following conditions: column, C18 silica gel; mobile phase, MeCN in Water (10 mmol/L NH₄HCO₃), 40% to 70% gradient in 40 min; detector, UV 254 nm. Afford tert-butyl N-[4-(bromomethyl)-3-fluoropyridin-2-yl]-N-(tert-butoxycarbonyl) carbamate (900 mg, 28%) as white solid. LCMS: (ESI, m/z): [M+1]⁺=405.10; ¹H NMR (300 MHz, Chloroform-d) δ 8.28 (d, J=4.9 Hz, 1H), 7.34-7.31 (m, 1H), 4.46 (m, 2H), 1.42 (s, 18H); ¹⁹F NMR (282 MHz, CDCl₃) δ, −130.81.

### Synthesis of Intermediate 4: Step 4

To a stirred mixture of tert-butyl N-[4-(bromomethyl)-3-fluoropyridin-2-yl]-N-(tert-butoxycarbonyl) carbamate (200 mg, 0.49 mmol, 1 equiv) and 4-methyl-5-nitropyridin-3-ylboronic acid (intermediate 6:107.75 mg, 0.59 mmol, 1.2 equiv) in dioxane (5 mL) were added K₂CO₃ (206.11 mg, 1.48 mmol, 3.0 equiv) and Pd(dppf)Cl₂ (40.20 mg, 0.049 mmol, 0.1 equiv) at room temperature under nitrogen atmosphere. The resulting mixture was stirred for 16 h at 80° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in water (10 mmol/L NH₄HCO₃), 45% to 65% gradient in 20 min; detector, UV 254/220 nm to afford tert-butyl N-(tert-butoxycarbonyl)-N-{3-fluoro-4-[(4-methyl-5-nitropyridin-3-yl)methyl]pyridin-2-yl}carbamate (110 mg, 48%) as a white solid. LCMS: (ESI, m/z): [M+1]⁺=463.2; ¹H NMR (400 MHz, Chloroform-d) δ 8.98 (s, 1H), 8.56 (s, 1H), 8.24 (d, 1H), 6.96-6.77 (m, 1H), 4.18 (s, 2H), 2.41 (m, 3H), 1.40 (s, 18H); LCMS: (ESI, m/z): [M+1]⁺=183.1; ¹H NMR (400 MHz, DMSO-d₆) δ 9.06 (s, 1H), 8.80 (s, 1H), 2.62 (s, 3H).

### Synthesis of Intermediate 4: Step 5

To a stirred mixture of tert-butyl N-(tert-butoxycarbonyl)-N-{3-fluoro-4-[(4-methyl-5-nitropyridin-3-yl)methyl]pyridin-2-yl}carbamate (100 mg, 0.22 mmol, 1 equiv) and iron (60.38 mg, 1.08 mmol, 5 equiv) in water (1 mL) and methanol (5 mL) was added NH₄Cl (115.66 mg, 2.16 mmol, 10.0 equiv) at room temperature under nitrogen atmosphere. The resulting mixture was stirred for 1 h at 60° C. under nitrogen atmosphere. Desired product could be detected by LCMS. The resulting mixture was concentrated under reduced pressure. The residue was purified by silica gel column chromatography, eluted with CH₂Cl₂/MeOH (10:1) to afford tert-butyl N-{4-[(5-amino-4-methylpyridin-3-yl)methyl]-3-fluoropyridin-2-yl}-N-(tert-butoxycarbonyl) carbamate (55 mg, 58%) as a white solid. LCMS: (ESI, m/z): [M+1]⁺=433.3; ¹H NMR (400 MHz, Chloroform-d) δ 8.15 (m, 1H), 8.07 (s, 1H), 7.86 (s, 1H), 6.85 (m, 1H), 4.03 (s, 2H), 1.99 (s, 3H), 1.42 (s, 18H).

### Intermediate 5

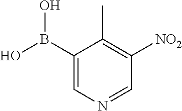

### Synthesis of Intermediate 5

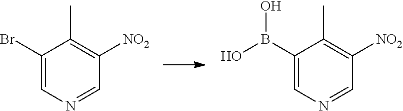

To a solution of 3-bromo-4-methyl-5-nitropyridine (5 g, 23.04 mmol, 1 equiv) and bis(pinacolato)diboron (8.78 g, 34.56 mmol, 1.5 equiv) in dioxane (100 mL) were added AcOK (6.78 g, 69.12 mmol, 3 equiv) and Pd(dppf)Cl₂ (1.69 g, 2.30 mmol, 0.1 equiv). After stirring for 16 h at 110° C. under a nitrogen atmosphere, the resulting mixture was concentrated under reduced pressure. Desired product could be detected by LCMS. The residue was purified by reversed-phase flash chromatography with the following conditions: column, C18 silica gel; mobile phase, MeCN (0.1% FA) in Water (0.1% FA), 10% to 30% gradient in 25 min; detector, UV 254/220 nm. This resulted in 4-methyl-5-nitropyridin-3-ylboronic acid (3.31 g, crude) as a white solid. LCMS: (ESI, m/z): [M+1]⁺=183.1; ¹H NMR (400 MHz, DMSO-d₆) δ 9.06 (s, 1H), 8.80 (s, 1H), 2.62 (s, 3H).

### Example 1

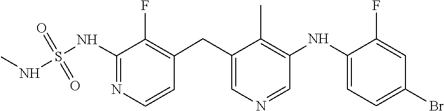 [SMILES: CNS(=O)(=O)Nc1nccc(Cc2cncc(Nc3ccc(Br)cc3F)c2C)c1F]

### Synthetic Route

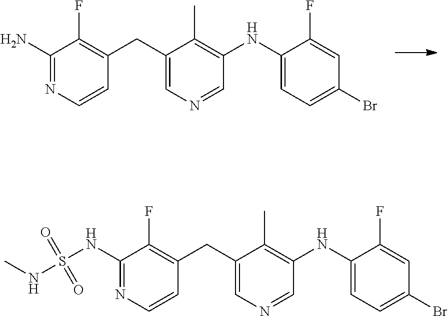 [SMILES: Cc1c(Cc2ccnc(N)c2F)cncc1Nc1ccc(Br)cc1F ; CNS(=O)(=O)Nc1nccc(Cc2cncc(Nc3ccc(Br)cc3F)c2C)c1F]

To a stirred solution of 5-[(2-amino-3-fluoropyridin-4-yl)methyl]-N-(4-bromo-2-fluorophenyl)-4-methylpyridin-3-amine (intermediate 1:60 mg, 0.15 mmol, 1 equiv) and pyridine (117.12 mg, 1.48 mmol, 10 equiv) in DMA (4 mL) was added N-methylsulfamoyl chloride (95.91 mg, 0.74 mmol, 5 equiv) in 0.5 mL DMA dropwise at room temperature under nitrogen atmosphere. The resulting mixture was stirred for 1 h at room temperature under nitrogen atmosphere. The residue was purified by reverse flash chromatography with the following conditions: column, C18 silica gel; mobile phase, MeCN in Water (10 mmol/L NH₄HCO₃), 10% to 50% gradient in 30 min; detector, UV 254/220 nm to afford N-(4-bromo-2-fluorophenyl)-5-({3-fluoro-2-[(methylsulfamoyl)amino]pyridin-4-yl}methyl)-4-methylpyridin-3-amine (32.5 mg, 44%) as a white solid. LCMS: [M+1]⁺=497.8; ¹H NMR (400 MHz, Methanol-d₄) δ 8.11 (m, 2H), 7.98 (m, 1H), 7.31 (m, 1H), 7.15 (m, 1H), 6.71 (m, 1H), 6.64 (m, 1H), 4.16 (s, 2H), 2.63 (s, 3H), 2.16 (s, 3H); ¹⁹F NMR (377 MHz, Methanol-d₄) δ −128.358, −142.365.

### Example 2

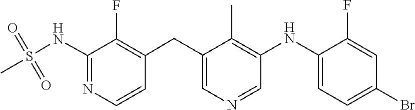 [SMILES: Cc1c(Cc2ccnc(NS(C)(=O)=O)c2F)cncc1Nc1ccc(Br)cc1F]

### Synthetic Route

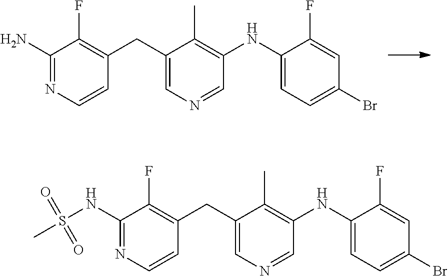 [SMILES: Cc1c(Cc2ccnc(N)c2F)cncc1Nc1ccc(Br)cc1F ; Cc1c(Cc2ccnc(NS(C)(=O)=O)c2F)cncc1Nc1ccc(Br)cc1F]

### Step 1

To a stirred solution of 5-[(2-amino-3-fluoropyridin-4-yl)methyl]-N-(4-bromo-2-fluorophenyl)-4-methylpyridin-3-amine (intermediate 1:20 mg, 0.049 mmol, 1 equiv) and Et₃N (49.94 mg, 0.49 mmol, 10 equiv) in THF (2 mL) was added MsCl (28.26 mg, 0.25 mmol, 5 equiv) in 0.5 mL THF dropwise at 0° C. under nitrogen atmosphere. The resulting mixture was stirred for 4 h at room temperature under nitrogen atmosphere. The reaction was monitored by LCMS. Desired product could be detected by LCMS. The resulting mixture was concentrated under reduced pressure. This resulted in N-[4-({5-[(4-bromo-2-fluorophenyl)amino]-4-methylpyridin-3-yl}methyl)-3-fluoropyridin-2-yl]-N-methanesulfonylmethanesulfonamide (100 mg, crude) as a brown yellow solid. The crude product was used in the next step directly without further purification. LCMS: [M+1]⁺=561.0

### Step 2

To a stirred solution of N-[4-({5-[(4-bromo-2-fluorophenyl)amino]-4-methylpyridin-3-yl}methyl)-3-fluoropyridin-2-yl]-N-methanesulfonylmethanesulfonamide (100 mg, crude) in MeOH (2 mL) was added NaOH (2 mL, 1 M in water) dropwise at 0° C. under nitrogen atmosphere. The resulting mixture was stirred for 30 min at room temperature under nitrogen atmosphere. The mixture was acidified to pH˜6 with HCl (2 M in water). The resulting mixture was extracted with EtOAc (3×10 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (15:1) to afford N-[4-({5-[(4-bromo-2-fluorophenyl)amino]-4-methylpyridin-3-yl}methyl)-3-fluoropyridin-2-yl]methanesulfonamide (12 mg, 92% purity) as a white solid. The residue was futher purified by reverse flash chromatography with the following conditions: column, C18 silica gel; mobile phase, MeCN in Water (0.1% FA), 5% to 75% gradient in 20 min; detector, UV 254 nm. This resulted in N-[4-({5-[(4-bromo-2-fluorophenyl)amino]-4-methylpyridin-3-yl}methyl)-3-fluoropyridin-2-yl]methanesulfonamide (6.1 mg, 99% purity) as a white solid. LCMS: [M+1]⁺=483.10; ¹H NMR (400 MHz, Methanol-d₄) δ 8.11 (m, 2H), 7.99 (m, 1H), 7.31 (m, 2.2 Hz, 1H), 7.15 (m, 1H), 6.76 (m, 1H), 6.63 m, 1H), 4.16 (s, 2H), 3.38 (s, 3H), 2.16 (s, 3H); ¹⁹F NMR (377 MHz, Methanol-d₄) δ −128.266, −140.621.

### Example 3

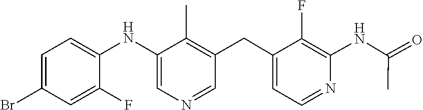 [SMILES: CC(=O)Nc1nccc(Cc2cncc(Nc3ccc(Br)cc3F)c2C)c1F]

### Synthetic Route

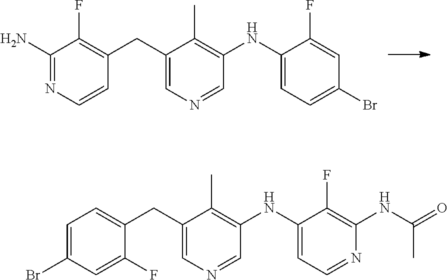 [SMILES: Cc1c(Cc2ccnc(N)c2F)cncc1Nc1ccc(Br)cc1F ; CC(=O)Nc1nccc(Nc2cncc(Cc3ccc(Br)cc3F)c2C)c1F]

To a stirred mixture of 5-[(2-amino-3-fluoropyridin-4-yl)methyl]-N-(4-bromo-2-fluorophenyl)-4-methylpyridin-3-amine (intermediate 1:10 mg, 0.025 mmol, 1 equiv) and pyridine (5.86 mg, 0.075 mmol, 3.0 equiv) in DCM (1 mL) was added acetyl chloride (1.94 mg, 0.025 mmol, 1.0 equiv) in DCM (0.1 mL) dropwise at 0° C. under nitrogen atmosphere. The resulting mixture was stirred for 1 h at 0° C. under nitrogen atmosphere. The resulting mixture was concentrated under reduced pressure. The residue was purified by reverse flash chromatography with the following conditions: column, C18 silica gel; mobile phase, MeCN in water (10 mmol/L NH₄HCO₃), 30% to 50% gradient in 20 min; detector, UV 254/220 nm to afford N-[4-({5-[(4-bromo-2-fluorophenyl)amino]-4-methylpyridin-3-yl}methyl)-3-fluoropyridin-2-yl]acetamide (2.1 mg, 18%) as a white solid. LCMS: (ESI, m/z): [M+1]⁺=446.80; ¹H NMR (400 MHz, Methanol-d₄) δ 8.11 (m, 3H), 7.31 (m, 1H), 7.15 (m, 1H), 6.97 (m, 1H), 6.63 (m, 1H), 4.20 (s, 2H), 2.19 (s, 3H), 2.16 (s, 3H); ¹⁹F NMR (377 MHz, Methanol-d₄) δ −128.331, −132.356

### Example 4

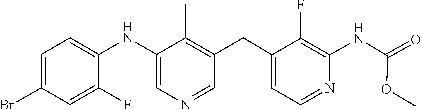 [SMILES: COC(=O)Nc1nccc(Cc2cncc(Nc3ccc(Br)cc3F)c2C)c1F]

### Synthetic Route

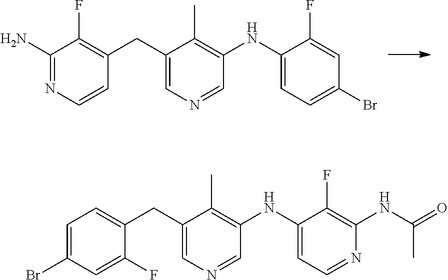 [SMILES: Cc1c(Cc2ccnc(N)c2F)cncc1Nc1ccc(Br)cc1F ; CC(=O)Nc1nccc(Nc2cncc(Cc3ccc(Br)cc3F)c2C)c1F]

To a stirred mixture of 5-[(2-amino-3-fluoropyridin-4-yl)methyl]-N-(4-bromo-2-fluorophenyl)-4-methylpyridin-3-amine (intermediate 1:10 mg, 0.025 mmol, 1 equiv) and pyridine (5.86 mg, 0.075 mmol, 3.0 equiv) in DCM (1 mL) was added methyl chloroformate (2.33 mg, 0.025 mmol, 1.0 equiv) in DCM (0.1 mL) dropwise at 0° C. under nitrogen atmosphere. The resulting mixture was stirred for 1 h at room temperature under nitrogen atmosphere. Desired product could be detected by LCMS. The resulting mixture was concentrated under reduced pressure. The residue was purified by reverse flash chromatography with the following conditions: column, C18 silica gel; mobile phase, MeCN in water (0.1% FA), 30% to 50% gradient in 20 min; detector, UV 254/220 nm to afford methyl N-[4-({5-[(4-bromo-2-fluorophenyl)amino]-4-methylpyridin-3-yl}methyl)-3-fluoropyridin-2-yl]carbamate (2.0 mg, 17%) as a white solid. LCMS: (ESI, m/z): [M+1]⁺=462.8. ¹H NMR (400 MHz, Methanol-d₄) δ 8.09 (m, 3H), 7.31 (m, 1H), 7.15 (m, 1H), 6.90 (m, 1H), 6.64 (m, 1H), 4.19 (s, 2H), 3.77 (s, 3H), 2.16 (s, 3H); ¹⁹F NMR (377 MHz, Methanol-d₄) δ −128.274, −135.542.

### Example 5

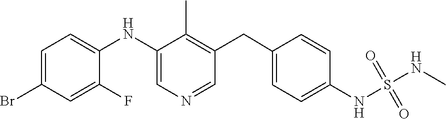 [SMILES: CNS(=O)(=O)Nc1ccc(Cc2cncc(Nc3ccc(Br)cc3F)c2C)cc1]

### Synthetic Route

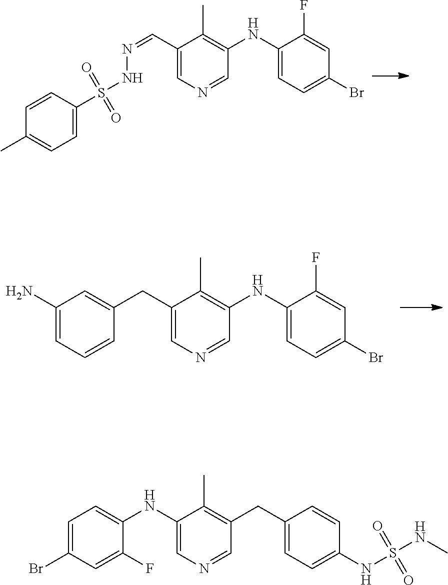 [SMILES: Cc1ccc(S(=O)(=O)N/N=C\c2cncc(Nc3ccc(Br)cc3F)c2C)cc1 ; Cc1c(Cc2cccc(N)c2)cncc1Nc1ccc(Br)cc1F ; CNS(=O)(=O)Nc1ccc(Cc2cncc(Nc3ccc(Br)cc3F)c2C)cc1]

Step 1: To a stirred mixture of N′-[(1Z)-{5-[(4-bromo-2-fluorophenyl)amino]-4-methylpyridin-3-yl}methylidene]-4-methylbenzenesulfonohydrazide (From Intermediate 1: Step 6 product: 25 mg, 0.052 mmol, 1 equiv) and m-aminophenylboronic acid (21.52 mg, 0.16 mmol, 3.0 equiv) in dioxane (2 mL) was added K₂CO₃ (8.69 mg, 0.062 mmol, 1.2 equiv) at room temperature under nitrogen atmosphere. The resulting mixture was stirred for 2 h at 110° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in water (10 mmol/L NH₄HCO₃), 40% to 70% gradient in 20 min; detector, UV 254/220 nm to afford 5-[(3-aminophenyl)methyl]-N-(4-bromo-2-fluorophenyl)-4-methylpyridin-3-amine (15 mg, 74%) as a white solid. LCMS: (ESI, m/z): [M+1]⁺=386.2; ¹H NMR (400 MHz, Methanol-d₄) δ 8.40 (s, 1H), 8.23 (s, 1H), 7.24 (m, 1H), 7.13-7.05 (m, 2H), 6.75-6.65 (m, 1H), 6.54 (m, 2H), 6.39 (m, 1H), 3.95 (m, 2H), 2.13 (s, 3H).

Step 2: To a stirred mixture of 5-[(3-aminophenyl)methyl]-N-(4-bromo-2-fluorophenyl)-4-methylpyridin-3-amine (15 mg, 0.039 mmol, 1 equiv) and pyridine (30.72 mg, 0.39 mmol, 10.0 equiv) in DMA (1 mL) was added N-methylsulfamoyl chloride (25.16 mg, 0.2 mmol, 5 equiv) in DMA (0.2 mL) dropwise at 0° C. under nitrogen atmosphere. The resulting mixture was stirred for 1 h at room temperature under nitrogen atmosphere. Desired product could be detected by LCMS. The residue was purified by reverse flash chromatography with the following conditions: column, C18 silica gel; mobile phase, MeCN in Water (10 mmol/L NH₄HCO₃), 30% to 50% gradient in 20 min; detector, UV 254/220 nm to afford N-(4-bromo-2-fluorophenyl)-4-methyl-5-({4-[(methylsulfamoyl)amino]phenyl}methyl)pyridin-3-amine (4.3 mg, 23%) as a white solid. LCMS: (ESI, m/z): [M+1]⁺=478.90; ¹H NMR (400 MHz, Methanol-d₄) δ 8.10 (m, 2H), 7.32-7.20 (m, 2H), 7.15-7.03 (m, 2H), 7.00 (s, 1H), 6.87 (m, 1H), 6.56 (m, 1H), 4.07 (s, 2H), 2.55 (m, 3H), 2.13 (s, 3H); ¹⁹F NMR (377 MHz, Methanol-d₄) δ −129.167.

### Example 6

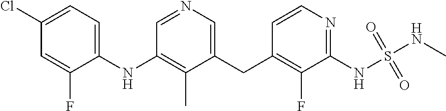 [SMILES: CNS(=O)(=O)Nc1nccc(Cc2cncc(Nc3ccc(Cl)cc3F)c2C)c1F]

### Synthetic Route

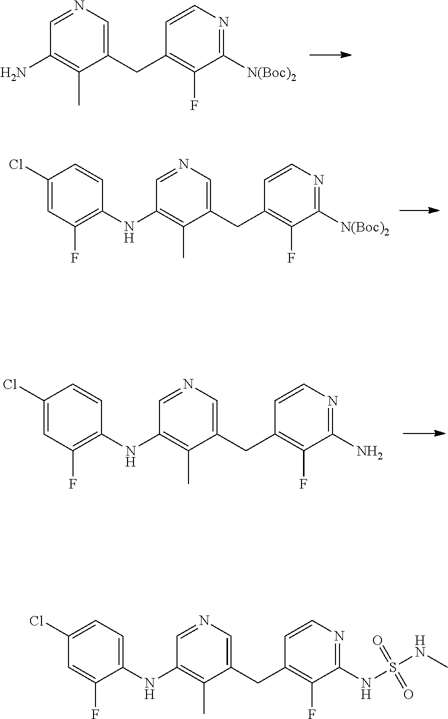

### Synthesis

Step1: To a stirred mixture of tert-butyl N-{4-[(5-amino-4-methylpyridin-3-yl)methyl]-3-fluoropyridin-2-yl}-N-(tert-butoxycarbonyl) carbamate (Intermediate 4:50 mg, 0.16 mmol, 1.0 equiv) and Cs₂CO₃ (75.3 mg, 0.23 mmol, 2.0 equiv) in toluene (1 mL) were added 4-chloro-2-fluoro-1-iodobenzene (44.47 mg, 0.17 mmol, 1.5 equiv) and Pd₂(dba)₃ (10.59 mg, 0.012 mmol, 0.1 equiv) and XantPhos (6.69 mg, 0.012 mmol, 0.1 equiv) at room temperature under nitrogen atmosphere. The resulting mixture was stirred for 4 h at 100° C. under nitrogen atmosphere. Desired product could be detected by LCMS. The resulting mixture was concentrated under reduced pressure. The residue was purified by silica gel column chromatography, eluted with PE/EA (1:1) to afford tert-butyl N-(tert-butoxycarbonyl)-N-[4-({5-[(4-chloro-2-fluorophenyl)amino]-4-methylpyridin-3-yl}methyl)-3-fluoropyridin-2-yl]carbamate (40 mg, 61%) as a yellow solid. LCMS: (ESI, m/z): [M+1]⁺=561.15; ¹H NMR (400 MHz, Chloroform-d) δ 8.41 (s, 1H), 8.23-8.15 (m, 2H), 7.15-7.12 (m, 1H), 7.00-6.97 (m, 1H), 6.92-6.89 (m, 1H), 6.77-6.72 (m, 1H), 5.42 (s, 1H), 4.10 (s, 2H), 2.12 (s, 3H), 1.42 (s, 18H); ¹⁹F NMR (377 MHz, CDCl₃) δ −130.06, −131.08.

Step 2: To a stirred solution of tert-butyl N-(tert-butoxycarbonyl)-N-[4-({5-[(4-chloro-2-fluorophenyl)amino]-4-methylpyridin-3-yl}methyl)-3-fluoropyridin-2-yl]carbamate (18 mg, 0.032 mmol, 1 equiv) in DCM (2 mL) was added TFA (0.60 mL, 8.06 mmol, 251.77 equiv) dropwise at room temperature under nitrogen atmosphere. The resulting mixture was stirred for 1 h at room temperature under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (10 mmol/L NH₄HCO₃), 40% to 80% gradient in 20 min; detector, UV 254/220 nm to afford 4-({5-[(4-chloro-2-fluorophenyl)amino]-4-methylpyridin-3-yl}methyl)-3-fluoropyridin-2-amine (10 mg, 86%) as a white solid. LCMS: (ESI, m/z): [M+1]⁺=361.1

Step 3: To a stirred mixture of 4-({5-[(4-chloro-2-fluorophenyl)amino]-4-methylpyridin-3-yl}methyl)-3-fluoropyridin-2-amine (10 mg, 0.028 mmol, 1 equiv) and pyridine (21.92 mg, 0.280 mmol, 10 equiv) in DMA (2 mL) was added N-methylsulfamoyl chloride (17.96 mg, 0.140 mmol, 5 equiv) in 0.5 mL of DMA dropwise at 0° C. under nitrogen atmosphere. The resulting mixture was stirred for 1 h at room temperature under nitrogen atmosphere. The residue was purified by reversed-phase flash chromatography with the following conditions: column, C18 silica gel; mobile phase, MeCN in Water (10 mmol/L NH₄HCO₃), 10% to 50% gradient in 20 min; detector, UV 254/220 nm to afford N-(4-chloro-2-fluorophenyl)-5-({3-fluoro-2-[(methylsulfamoyl)amino]pyridin-4-yl}methyl)-4-methylpyridin-3-amine (5.1 mg, 40%) as a white solid. LCMS: (ESI, m/z): [M+1]⁺=454.2; ¹H NMR (400 MHz, Me thanol-d₄) δ 8.09 (m, 2H), 7.97 (m, 1H), 7.19 (m, 1H), 7.02 (m, 1H), 6.75-6.64 (m, 2H), 4.15 (s, 2H), 2.63 (s, 3H), 2.16 (s, 3H); ¹⁹F NMR (377 MHz, Methanol-d₄) δ −128.109, −142.314.

### Example 7

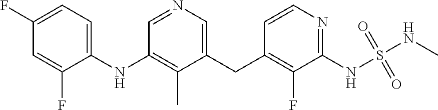 [SMILES: CNS(=O)(=O)Nc1nccc(Cc2cncc(Nc3ccc(F)cc3F)c2C)c1F]

### Synthetic Route

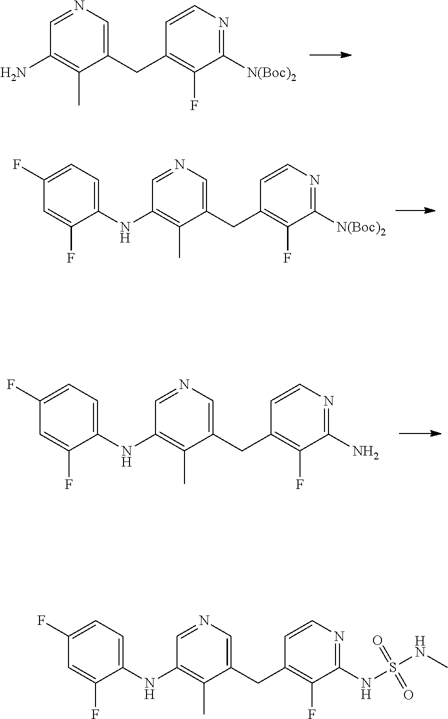

Step 1: To a solution of tert-butyl N-{4-[(5-amino-4-methylpyridin-3-yl)methyl]-3-fluoropyridin-2-yl}-N-(tert-butoxycarbonyl) carbamate (Intermediate 4:50 mg, 0.12 mmol, 1.0 equiv) and 2,4-difluoro-1-iodobenzene (42 mg, 0.17 mmol, 1.5 equiv) in dioxane (0.5 mL) were added Cs₂CO₃ (75 mg, 0.23 mmol, 2.0 equiv), Pd₂(dba)₃ (11 mg, 0.012 mmol, 0.1 equiv) and XantPhos (7 mg, 0.012 mmol, 0.1 equiv) under nitrogen atmosphere. And then keep stirring for 16 h at 80° C. Desired product could be detected by LCMS. The resulting mixture was concentrated under reduced pressure. The residue was purified by silica gel column chromatography, eluted with PE/EA (1:1) to afford tert-butyl N-(tert-butoxycarbonyl)-N-[4-({5-[(2,4-difluorophenyl)amino]-4-methylpyridin-3-yl}methyl)-3-fluoropyridin-2-yl]carbamate (15 mg, 23%) as a light yellow solid. LCMS: (ESI, m/z): [M+1]⁺=545.30; ¹H NMR (400 MHz, Chloroform-d) δ8.25 (s, 1H), 8.20 (d, J=5.0 Hz, 1H), 8.11 (s, 1H), 6.98-6.89 (m, 3H), 6.84-6.80 (m, 1H), 4.11 (s, 2H), 2.16 (s, 3H), 1.43 (s, 18H).

Step 2: To a stirred solution of tert-butyl N-(tert-butoxycarbonyl)-N-[4-({5-[(2,4-difluorophenyl)amino]-4-methylpyridin-3-yl}methyl)-3-fluoropyridin-2-yl]carbamate (15 mg, 0.028 mmol, 1.0 equiv) in DCM (1 mL) was added TFA (0.2 mL) at 0° C., and then keep stirring for 1 h at room temperature. The resulting mixture was concentrated under reduced pressure. Desired product could be detected by LCMS. The residue was neutralized to pH 10 with saturated NaHCO₃ (aq.). The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 4-({5-[(2,4-difluorophenyl)amino]-4-methylpyridin-3-yl}methyl)-3-fluoropyridin-2-amine (8 mg, 84%) as a white solid. LCMS: (ESI, m/z): [M+1]⁺=345.00; ¹H NMR (300 MHz, Chloroform-d) δ 8.30 (s, 1H), 8.14 (s, 1H), 7.73 (d, J=5.2 Hz, 1H), 6.95-6.72 (m, 3H), 6.28-6.25 (m, 1H), 4.64 (s, 2H), 3.99 (s, 2H), 2.14 (s, 3H); ¹⁹F NMR (282 MHz, Chloroform-d) δ −119.100, −126.990, −145.330.

Step 3: To a stirred solution of 4-({5-[(2,4-difluorophenyl)amino]-4-methylpyridin-3-yl}methyl)-3-fluoropyridin-2-amine (5 mg, 0.015 mmol, 1.0 equiv) and pyridine (11.49 mg, 0.15 mmol, 10 equiv) in DMA (0.5 mL) was added N-methylsulfamoyl chloride (9.41 mg, 0.075 mmol, 5 equiv) in DMA (0.2 mL) dropwise at 0° C. under air atmosphere. And then keep stirring for 1 h at room temperature. Desired product could be detected by LCMS. The resulting reaction mixture was purified by reversed-phase flash chromatography with the following conditions: column, C18 silica gel; mobile phase, MeCN in Water (10 mmol/L NH₄HCO₃), 5% to 60% gradient in 40 min; detector, UV254 nm. Afford N-(2,4-difluorophenyl)-5-({3-fluoro-2-[(methylsulfamoyl)amino]pyridin-4-yl}methyl)-4-methylpyridin-3-amine (2.2 mg, 34%) as white solid. LCMS: (ESI, m/z): [M+1]⁺=438.05; 1H NMR (400 MHz, Methanol-d₄) δ 8.01-7.94 (m, 2H), 7.94-7.90 (m, 1H), 7.04-6.98 (m, 1H), 6.97-6.81 (m, 2H), 6.70-6.67 (m, 1H), 4.14 (s, 2H), 2.63 (s, 3H), 2.17 (s, 3H); ¹⁹F NMR (377 MHz, Methanol-d₄) δ −120.464, −123.894, −142.404.

### Example 8

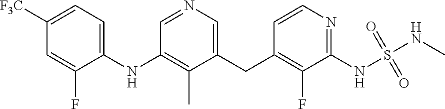 [SMILES: CNS(=O)(=O)Nc1nccc(Cc2cncc(Nc3ccc(C(F)(F)F)cc3F)c2C)c1F]

### Synthetic Route

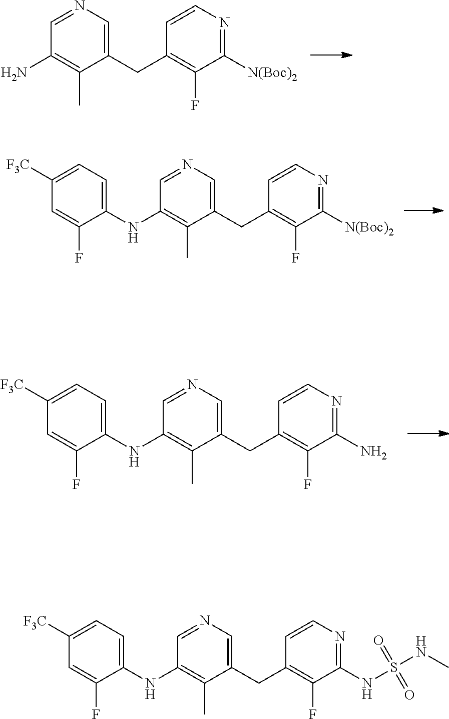

Step 1: A mixture of tert-butyl N-{4-[(5-amino-4-methylpyridin-3-yl)methyl]-3-fluoropyridin-2-yl}-N-(tert-butoxycarbonyl) carbamate (Intermediate 4:50 mg, 0.12 mmol, 1.0 equiv), 2-fluoro-1-iodo-4-(trifluoromethyl)benzene (50 mg, 0.17 mmol, 1.5 equiv), Cs₂CO₃ (75.33 mg, 0.23 mmol, 2.0 equiv), Pd₂(dba)₃ (10.59 mg, 0.012 mmol, 0.1 equiv) and XantPhos (6.69 mg, 0.012 mmol, 0.1 equiv) in dioxane (5.00 mL) was stirred for 16 h at 80° C. under nitrogen atmosphere. Desired product could be detected by LCMS. The resulting mixture was concentrated under reduced pressure. The residue was purified by silica gel column chromatography, eluted with PE/EA (5:1) to afford tert-butyl N-(tert-butoxycarbonyl)-N-{3-fluoro-4-[(5-{[2-fluoro-4-(trifluoromethyl)phenyl]amino}-4-methylpyridin-3-yl)methyl]pyridin-2-yl}carbamate (40 mg, 58%) as a yellow solid. LCMS: [M+1]⁺=595.40; ¹H NMR (300 MHz, Chloroform-d) δ 8.50 (s, 1H), 8.32-8.20 (m, 2H), 7.40 (d, J=11.2 Hz, 2H), 6.97-6.94 (m, 1H), 6.84-6.78 (m, 1H), 5.84 (s, 1H), 4.16 (s, 2H), 2.20 (s, 3H), 1.45 (s, 18H).

Step 2: To a stirred solution of tert-butyl N-(tert-butoxycarbonyl)-N-{3-fluoro-4-[(5-{[2-fluoro-4-(trifluoromethyl)phenyl]amino}-4-methylpyridin-3-yl)methyl]pyridin-2-yl}carbamate (40 mg, 0.067 mmol, 1.0 equiv) in DCM (0.5 mL) was added TFA (0.1 mL) dropwise at 0° C. under air atmosphere. The resulting mixture was stirred for additional 1 h at room temperature. Desired product could be detected by LCMS. The residue was neutralized to pH 10 with saturated NaHCO₃ (aq.). The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with MeOH/DCM (1:10) to afford 3-fluoro-4-[(5-{[2-fluoro-4-(trifluoromethyl)phenyl]amino}-4-methylpyridin-3-yl)methyl]pyridin-2-amine (20 mg, 75%) as a yellow solid. LCMS: [M+1]⁺=395.10; ¹H NMR (300 MHz, Chloroform-d) δ 8.50 (s, 1H), 8.32 (s, 1H), 7.75 (d, J=5.4 Hz, 1H), 7.37 (d, J=11.3 Hz, 1H), 7.24 (s, 1H), 6.73 (t, J=8.4 Hz, 1H), 6.32 (t, J=5.2 Hz, 1H), 5.76 (s, 1H), 5.00 (s, 2H), 4.06 (s, 2H), 2.19 (s, 3H).

Step 3: To a stirred solution of 3-fluoro-4-[(5-{[2-fluoro-4-(trifluoromethyl)phenyl]amino}-4-methylpyridin-3-yl)methyl]pyridin-2-amine (10 mg, 0.025 mmol, 1.0 equiv) and Pyridine (20.06 mg, 0.250 mmol, 10 equiv) in DMA (1.00 mL) was added N-methylsulfamoyl chloride (16.43 mg, 0.13 mmol, 5 equiv) in DMA (0.2 mL) dropwise at 0° C. under nitrogen atmosphere. The resulting mixture was stirred for additional 1 h at room temperature. Desired product could be detected by LCMS. The residue was purified by reversed-phase flash chromatography with the following conditions: column, C18 silica gel; mobile phase, MeCN in Water (10 mmol/L NH₄HCO₃), 30% to 70% gradient in 30 min; detector, UV 254 nm. Afford 5-({3-fluoro-2-[(methylsulfamoyl)amino]pyridin-4-yl}methyl)-N-[2-fluoro-4-(trifluoromethyl)phenyl]-4-methylpyridin-3-amine (2.2 mg, 17%) as a white solid. LCMS: [M+1]⁺=488.05; ¹H NMR (300 MHz, Methanol-d₄) δ 8.30-8.25 (m, 2H), 8.02-8.00 (m, 1H), 7.43-7.39 (m, 1H), 7.33-7.30-7.25 (m, 1H), 6.77-6.74 (m, 1H), 6.66-6.60 (m, 1H), 4.21 (s, 2H), 2.65 (s, 3H), 2.19 (s, 3H); ¹⁹F NMR (282 MHz, Methanol-d₄) δ −62.898, −132.660, −142.345.

### Example 9

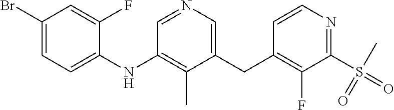 [SMILES: Cc1c(Cc2ccnc(S(C)(=O)=O)c2F)cncc1Nc1ccc(Br)cc1F]

### Synthetic Route

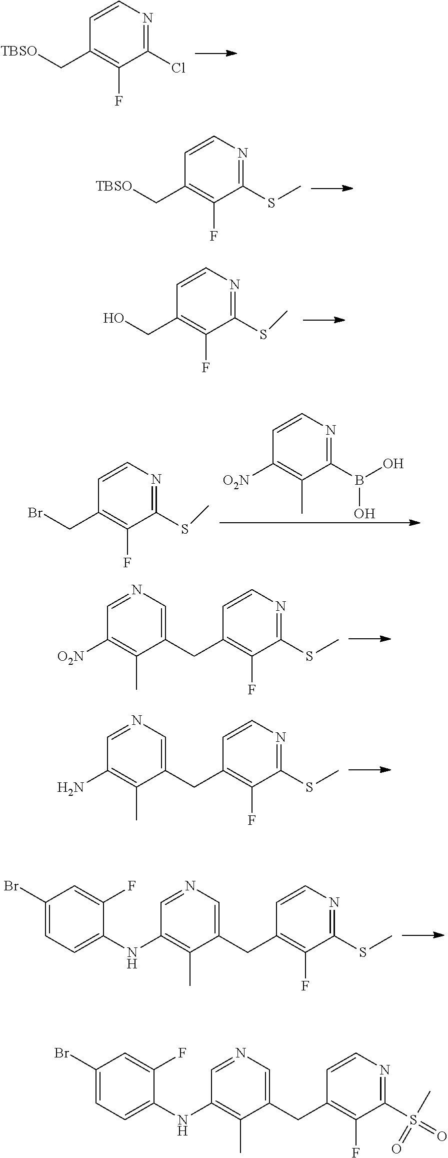

Step 1: To a stirred solution of 4-{[(tert-butyldimethylsilyl)oxy]methyl}-2-chloro-3-fluoropyridine (Intermediate 3:3 g, 10.88 mmol, 1 equiv) in THF was added (methylsulfanyl) sodium (0.76 g, 10.88 mmol, 1 equiv) at 0° C. The resulting mixture was stirred for 16 h at room temperature. The resulting mixture was concentrated under reduced pressure. The residue was purified by reversed-phase flash chromatography with the following conditions: column, C18 silica gel; mobile phase, MeCN in Water (0.1% TFA), 10% to 80% gradient in 30 min; detector, UV 254 nm. This resulted in mixture of 4-{[(tert-butyldimethylsilyl)oxy]methyl}-3-fluoro-2-(methylsulfanyl)pyridine and (3-fluoro-2-(methylthio)pyridin-4-yl) methanol (877 mg) as a white solid. LCMS: (ESI, m/z): [M+1]⁺=288.0

Step 2: To a stirred solution of 4-{[(tert-butyldimethylsilyl)oxy]methyl}-3-fluoro-2-(methylsulfanyl)pyridine (100 mg, 0.35 mmol, 1 equiv) in THF (0.5 mL) were added TBAF (90.95 mg, 0.35 mmol, 1 equiv) in THF (0.5 mL) dropwise at 0° C. under nitrogen atmosphere. The resulting mixture was stirred for 16 h at room temperature under nitrogen atmosphere. Desired product could be detected by LCMS. The resulting mixture was concentrated under reduced pressure. The resulting mixture was extracted with EtOAc (10×3 mL). The combined organic layers were washed with brine (10 mL×3), dried over anhydrous Na₂SO₄. After filtration, the filtrate was concentrated under reduced pressure. The residue was purified by silica gel column chromatography, eluted with PE/EA (5:1) to afford [3-fluoro-2-(methylsulfanyl)pyridin-4-yl]methanol (50 mg, 83%) as a white solid. LCMS: (ESI, m/z): [M+1]⁺=174.0: ¹H NMR (400 MHz, Chloroform-d) δ 8.27 (m, 1H), 7.16 (m, 1H), 4.78 (s, 2H), 2.58 (s, 3H): ¹⁹F NMR (377 MHz, Chloroform-d) δ −128.70, −128.96, −129.01, −129.17, −129.38.

Step 3: To a stirred solution of [3-fluoro-2-(methylsulfanyl)pyridin-4-yl]methanol (93 mg, 0.54 mmol, 1 equiv) and PPh₃ (211.24 mg, 0.81 mmol, 1.50 equiv) in DCM (1 mL) were added CBr₄ (267.09 mg, 0.81 mmol, 1.5 equiv) at 0° C. under nitrogen atmosphere. The resulting mixture was stirred for 16 h at room temperature under nitrogen atmosphere. The resulting mixture was concentrated under reduced pressure. The residue was purified by silica gel column chromatography, eluted with PE/EA (5:1) to afford 4-(bromomethyl)-3-fluoro-2-(methylsulfanyl)pyridine (20 mg, 16%) as a white solid. LCMS: (ESI, m/z): [M+1]⁺=235.0; ¹H NMR (300 MHz, Chloroform-d) δ 8.24 (d, J=5.0 Hz, 1H), 7.04 (t, J=5.2 Hz, 1H), 4.41 (d, J=1.0 Hz, 2H), 2.58 (s, 3H); ¹⁹F NMR (282 MHz, Chloroform-d) δ −127.40.

Step 4: To a stirred solution of 4-(bromomethyl)-3-fluoro-2-(methylsulfanyl)pyridine (20 mg, 0.085 mmol, 1 equiv) Pd(dppf)Cl₂·CH₂Cl₂ (6.90 mg, 0.009 mmol, 0.1 equiv) and K₂CO₃ (35.12 mg, 0.26 mmol, 3 equiv) in dioxane (5 mL) was added 4-methyl-5-nitropyridin-3-ylboronic acid (18.49 mg, 0.102 mmol, 1.2 equiv) at room temperature under nitrogen atmosphere. The resulting mixture was stirred for 16 h at 80° C. under nitrogen atmosphere. The residue was purified by silica gel column chromatography, eluted with CH₂Cl₂/MeOH (10:1) to afford 3-fluoro-4-[(4-methyl-5-nitropyridin-3-yl)methyl]-2-(methylsulfanyl)pyridine (6 mg, 24%) as a white solid. LCMS: (ESI, m/z): [M+1]⁺=294.3; ¹H NMR (400 MHz, Chloroform-d) δ 8.97 (s, 1H), 8.58 (s, 1H), 8.19 (d, J=5.0 Hz, 1H), 6.59 (t, J=5.2 Hz, 1H), 4.10 (s, 2H), 2.59 (s, 3H), 2.43 (s, 3H); ¹⁹F NMR (377 MHz, CDCl₃) δ −126.41, −126.67.

Step 5: To a stirred solution of 3-fluoro-4-[(4-methyl-5-nitropyridin-3-yl)methyl]-2-(methylsulfanyl)pyridine (82 mg, 0.28 mmol, 1 equiv) and H₂O (0.4 mL) in MeOH (1.6 mL) were added Fe (78.06 mg, 1.40 mmol, 5 equiv) and NH₄Cl (149.54 mg, 2.80 mmol, 10 equiv at room temperature. The resulting mixture was stirred for 2 h at 60° C. Desired product could be detected by LCMS. The resulting mixture was concentrated under reduced pressure. The residue was purified by silica gel column chromatography, eluted with PE/EA (10:1) to afford 5-{[3-fluoro-2-(methylsulfanyl)pyridin-4-yl]methyl}-4-methylpyridin-3-amine (54 mg, 73%) as a white solid. LCMS: (ESI, m/z): [M+1]⁺=264.10; ¹H NMR (300 MHz, Chloroform-d) δ 8.28-7.70 (m, 3H), 6.55 (t, J=5.2 Hz, 1H), 3.95 (s, 2H), 3.69 (d, J=25.6 Hz, 2H), 2.58 (s, 3H), 1.99 (s, 3H); ¹⁹F NMR (282 MHz, Chloroform-d) δ −127.34.

Step 6: To a stirred solution of 5-{[3-fluoro-2-(methylsulfanyl)pyridin-4-yl]methyl}-4-methylpyridin-3-amine (20 mg, 0.076 mmol, 1 equiv) Pd₂(dba)₃ (6.95 mg, 0.008 mmol, 0.1 equiv) Cs₂CO₃ (49.49 mg, 0.15 mmol, 2 equiv) and XantPhos (4.39 mg, 0.008 mmol, 0.1 equiv) in Toluene (1 mL) were added 4-bromo-2-fluoro-1-iodobenzene (34.28 mg, 0.11 mmol, 1.5 equiv) at room temperature under nitrogen atmosphere. The resulting mixture was stirred for 2 h at 100° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (0.1% NH₃·H₂O), 10% to 60% gradient in 30 min; detector, UV 254 nm. This resulted in N-(4-bromo-2-fluorophenyl)-5-{[3-fluoro-2-(methylsulfanyl)pyridin-4-yl]methyl}-4-methylpyridin-3-amine (14 mg, 42%) as a white solid. LCMS: (ESI, m/z): [M+1]⁺=436.01H NMR (400 MHz, Chloroform-d) δ 8.36 (s, 1H), 8.17 (d, J=5.4 Hz, 2H), 7.31-7.27 (m, 1H), 7.14 (d, J=8.5 Hz, 1H), 6.73 (t, J=8.7 Hz, 1H), 6.61 (t, J=5.2 Hz, 1H), 5.46 (s, 1H), 4.04 (s, 2H), 2.59 (s, 3H), 2.16 (s, 3H); ¹⁹F NMR (377 MHz, Chloroform-d) δ −126.97, −129.52.

Step 7: Into a 8 mL round-bottom flask were added N-(4-bromo-2-fluorophenyl)-5-{[3-fluoro-2-(methylsulfanyl)pyridin-4-yl]methyl}-4-methylpyridin-3-amine (12 mg, 0.028 mmol, 1 equiv) and acetone (1 mL)/H₂O (1 mL)/MeOH (0.1 mL) at room temperature. To the above mixture was added oxone (18.50 mg, 0.112 mmol, 4 equiv) in portions over 30 min at room temperature. The resulting mixture was stirred for additional 16 h at room temperature. The residue was purified by reversed-phase flash chromatography with the following conditions: column, C18 silica gel; mobile phase, MeCN in Water (0.1% FA), 10% to 65% gradient in 30 min; detector, UV 254 nm. This resulted in N-(4-bromo-2-fluorophenyl)-5-[(3-fluoro-2-methanesulfonylpyridin-4-yl)methyl]-4-methylpyridin-3-amine (4.3 mg, 33%) as a white solid. LCMS: (ESI, m/z): [M+1]⁺=467.85; ¹H NMR (400 MHz, Chloroform-d) δ 8.42 (s, 1H), 8.36 (m, 1H), 8.19 (s, 1H), 7.34-7.26 (m, 1H), 7.19-7.12 (m, 2H), 6.79-6.77 (m, 1H), 5.48 (s, 1H), 4.17 (s, 2H), 3.38 (s, 3H), 2.16 (m, 3H); ¹⁹F NMR (377 MHz, Chloroform-d) δ −124.114, −129.373.

### Example 10

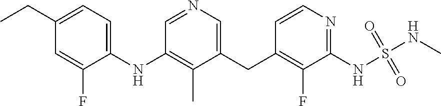 [SMILES: CCc1ccc(Nc2cncc(Cc3ccnc(NS(=O)(=O)NC)c3F)c2C)c(F)c1]

### Synthetic Route

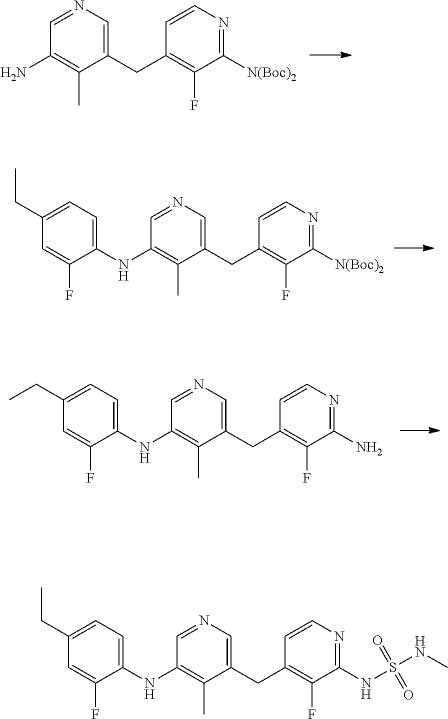

Step 1: To a stirred solution of tert-butyl N-{4-[(5-amino-4-methylpyridin-3-yl)methyl]-3-fluoropyridin-2-yl}-N-(tert-butoxycarbonyl) carbamate (Intermediate 4:50 mg, 0.12 mmol, 1 equiv) Cs₂CO₃ (75.33 mg, 0.23 mmol, 2 equiv) Pd₂(dba)₃ (10.59 mg, 0.012 mmol, 0.1 equiv) and XantPhos (6.69 mg, 0.012 mmol, 0.1 equiv) in dioxane (1 mL) were added 1-bromo-4-ethyl-2-fluorobenzene (35.21 mg, 0.17 mmol, 1.5 equiv) in dioxane (1 mL) dropwise at room temperature under nitrogen atmosphere. The resulting mixture was stirred for 2 h at 100° C. under nitrogen atmosphere. Desired product could be detected by LCMS. The resulting mixture was concentrated under reduced pressure. The residue was purified by silica gel column chromatography, eluted with PE/EA (1:1) to afford tert-butyl N-(tert-butoxycarbonyl)-N-[4-({5-[(4-ethyl-2-fluorophenyl)amino]-4-methylpyridin-3-yl}methyl)-3-fluoropyridin-2-yl]carbamate (34 mg, 53%) as a yellow oil. LCMS: (ESI, m/z): [M+1]⁺=555.1; ¹H NMR (400 MHz, Chloroform-d) δ 8.20 (m, 3H), 6.98 (m, 1H), 6.91 (m, 3H), 5.38 (s, 1H), 4.11 (s, 2H), 2.62 (m, 2H), 2.16 (s, 3H), 1.43 (s, 18H), 1.24 (m, 3H); ¹⁹F NMR (377 MHz, Chloroform-d) δ −130.60, −131.03.

Step 2: To a stirred solution of tert-butyl N-(tert-butoxycarbonyl)-N-[4-({5-[(4-ethyl-2-fluorophenyl)amino]-4-methylpyridin-3-yl}methyl)-3-fluoropyridin-2-yl]carbamate (30 mg, 0.054 mmol, 1 equiv) in DCM (2 mL) were added TFA (0.5 mL) dropwise at 0° C. The resulting mixture was stirred for 2 h at room temperature. Desired product could be detected by LCMS. The resulting mixture was concentrated under reduced pressure. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with CH₂Cl₂/MeOH (10:1) to afford 4-({5-[(4-ethyl-2-fluorophenyl)amino]-4-methylpyridin-3-yl}methyl)-3-fluoropyridin-2-amine (17 mg, 89%) as a white solid. LCMS: (ESI, m/z): [M+1]⁺=355.4; ¹H NMR (400 MHz, Chloroform-d) δ 8.22 (d, J=88.8 Hz, 2H), 7.74 (d, J=5.2 Hz, 1H), 7.00-6.94 (m, 1H), 6.91-6.83 (m, 2H), 6.27 (t, J=5.1 Hz, 1H), 5.35 (s, 1H), 4.67 (s, 2H), 4.00 (s, 2H), 2.61 (q, J=7.6 Hz, 2H), 2.17 (s, 3H), 1.23 (t, J=7.6 Hz, 3H); ¹⁹F NMR (377 MHz, Chloroform-d) δ −131.45, −145.22.

Step 3: To a stirred solution of 4-({5-[(4-ethyl-2-fluorophenyl)amino]-4-methylpyridin-3-yl}methyl)-3-fluoropyridin-2-amine (14 mg, 0.04 mmol, 1 equiv) in DMA (0.5 mL) were added Pyridine (31.25 mg, 0.40 mmol, 10 equiv) and N-methylsulfamoyl chloride (25.59 mg, 0.20 mmol, 5 equiv) in DMA (0.5 mL) dropwise at 0° C. The resulting mixture was stirred for 1 h at room temperature. Desired product could be detected by LCMS. The residue was purified by reversed-phase flash chromatography with the following conditions: column, C18 silica gel; mobile phase, MeCN in Water (10 mmol/L NH₄HCO₃), 10% to 80% gradient in 30 min; detector, UV 254 nm. This resulted in N-(4-ethyl-2-fluorophenyl)-5-({3-fluoro-2-[(methylsulfamoyl)amino]pyridin-4-yl}methyl)-4-methylpyridin-3-amine (4.6 mg, 26%) as a white solid. LCMS: (ESI, m/z): [M+1]⁺=448.05; ¹H NMR (400 MHz, Chloroform-d) δ 8.33 (s, 1H), 8.08 (s, 1H), 7.96 (d, J=5.2 Hz, 1H), 7.28-7.26 (m, 1H), 7.03-6.83 (m, 3H), 6.61-6.59 (m, 1H), 5.48 (s, 1H), 5.37 (s, 1H), 4.06 (s, 2H), 2.78 (d, J=3.2 Hz, 3H), 2.64-2.59 (m, 2H), 2.17 (s, 3H), 1.23 (t, J=7.6 Hz, 3H); ¹⁹F NMR (377 MHz, Chloroform-d) δ −130.867, −142.765.

### Example 11

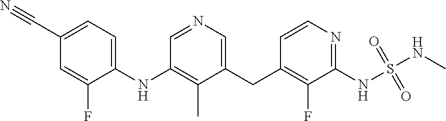 [SMILES: CNS(=O)(=O)Nc1nccc(Cc2cncc(Nc3ccc(C#N)cc3F)c2C)c1F]

### Synthetic Route

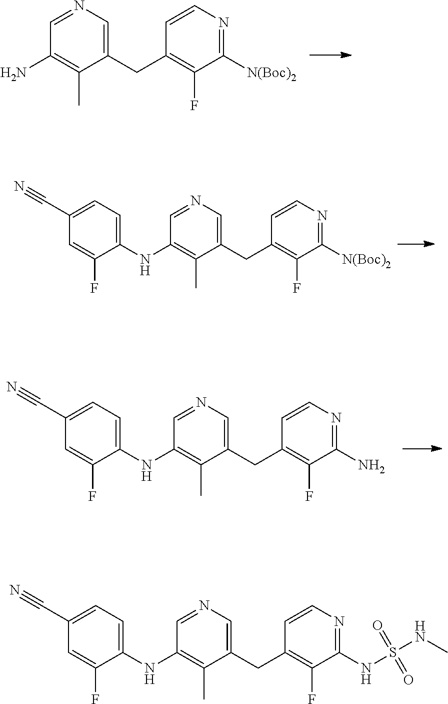

Step 1: To a stirred solution of tert-butyl N-{4-[(5-amino-4-methylpyridin-3-yl)methyl]-3-fluoropyridin-2-yl}-N-(tert-butoxycarbonyl) carbamate (Intermediate 4:50 mg, 0.115 mmol, 1 equiv) Cs₂CO₃ (90.41 mg, 0.23 mmol, 2.0 equiv) Pd₂(dba)₃ (10.59 mg, 0.013 mmol, 0.1 equiv) and XantPhos (6.69 mg, 0.013 mmol, 0.1 equiv) in dioxane were added 3-fluoro-4-iodobenzonitrile (42.84 mg, 0.173 mmol, 1.5 equiv) at room temperature under nitrogen atmosphere. The resulting mixture was stirred for 16 h at 80° C. under nitrogen atmosphere. The resulting mixture was concentrated under reduced pressure. The residue was purified by silica gel column chromatography, eluted with PE/EA (2:1) to afford tert-butyl N-(tert-butoxycarbonyl)-N-[4-({5-[(4-cyano-2-fluorophenyl)amino]-4-methylpyridin-3-yl}methyl)-3-fluoropyridin-2-yl]carbamate (38 mg, 59%) as a brown solid. LCMS: (ESI, m/z): [M+1]⁺=552.2: ¹H NMR (400 MHz, Chloroform-d) δ 8.48 (m, 1H), 8.32 (m, 1H), 8.21 (m, 1H), 7.36 (m, 2H), 6.93 (m), 6.61 (m, 1H), 5.99 (s, 1H), 4.15-4.12 (m, 2H), 2.14 (s, 3H), 1.43 (s, 18H); ¹⁹F NMR (377 MHz, Chloroform-d) δ −130.99, −133.12.

Step 2: To a stirred solution of tert-butyl N-(tert-butoxycarbonyl)-N-[4-({5-[(4-cyano-2-fluorophenyl)amino]-4-methylpyridin-3-yl}methyl)-3-fluoropyridin-2-yl]carbamate (50 mg, 0.091 mmol, 1 equiv) in DCM (5 mL) were added TFA (1 mL, 13.46 mmol, 148.52 equiv) dropwise at 0° C. under nitrogen atmosphere. The resulting mixture was stirred for 1 h at room temperature. Desired product could be detected by LCMS. The resulting mixture was concentrated under reduced pressure. The mixture was basified to pH 10 with NaHCO₃. The resulting mixture was extracted with EtOAc (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with CH₂Cl₂/MeOH (8:1) to afford 4-({5-[(2-amino-3-fluoropyridin-4-yl)methyl]-4-methylpyridin-3-yl}amino)-3-fluorobenzonitrile (20 mg, 63%) as a brown solid. LCMS: (ESI, m/z): [M+1]⁺=352.4; ¹H NMR (400 MHz, Chloroform-d) δ 8.41 (m, 2H), 7.73 (m, 1H), 7.37 (m, 1H), 7.28 (m, 1H), 6.61 (m, 1H), 6.29 (m, 1H), 5.91 (m, 1H), 4.89 (s, 2H), 4.03 (s, 2H), 2.16 (s, 3H); ¹⁹F NMR (377 MHz, 400 MHz, Chloroform-d) δ −133.33, −144.70.

Step 3: To a stirred solution of 4-({5-[(2-amino-3-fluoropyridin-4-yl)methyl]-4-methylpyridin-3-yl}amino)-3-fluorobenzonitrile (20 mg, 0.057 mmol, 1 equiv) in DMA (0.5 mL) were added pyridine (45.02 mg, 0.570 mmol, 10 equiv) and N-methylsulfamoyl chloride (36.87 mg, 0.285 mmol, 5 equiv) in DMA (0.5 mL) dropwise at 0° C. The resulting mixture was stirred for 1 h at room temperature. The residue was purified by reversed-phase flash chromatography with the following conditions: column, C18 silica gel; mobile phase, MeCN in Water (0.1% FA), 10% to 50% gradient in 30 min; detector, UV 254 nm. This resulted in 3-fluoro-4-{[5-({3-fluoro-2-[(methylsulfamoyl)amino]pyridin-4-yl}methyl)-4-methylpyridin-3-yl]amino}benzonitrile (2.4 mg, 10%) as a white solid. LCMS: (ESI, m/z): [M+1]⁺=445.1; ¹H NMR (400 MHz, Methanol-d₄) δ 8.33-8.28 (m, 2H), 8.01-7.98 (m, 1H), 7.52-7.49 (m, 1H), 7.37-7.30 (m, 1H), 6.76-6.73 (m, 1H), 6.62-6.58 (m, 1H), 4.21 (s, 2H), 2.63 (s, 3H), 2.20 (s, 3H); ¹⁹ F NMR (377 MHz, Methanol-d₄) δ −131.916, −142.205.12

### Example 12

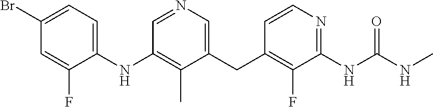 [SMILES: CNC(=O)Nc1nccc(Cc2cncc(Nc3ccc(Br)cc3F)c2C)c1F]

### Synthetic Route

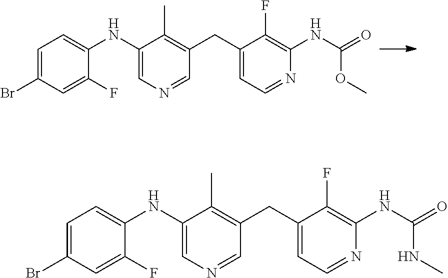 [SMILES: COC(=O)Nc1nccc(Cc2cncc(Nc3ccc(Br)cc3F)c2C)c1F ; CNC(=O)Nc1nccc(Cc2cncc(Nc3ccc(Br)cc3F)c2C)c1F]

A solution of methyl N-[4-({5-[(4-bromo-2-fluorophenyl)amino]-4-methylpyridin-3-yl}methyl)-3-fluoropyridin-2-yl]carbamate (Example 4, 25 mg, 0.054 mmol, 1 equiv) and CH₃NH₂ (2 M in THF, 5 mL, 10 mmol, 185.3 equiv) was irradiated with microwave radiation for 1 h at 80° C. LCMS showed ˜30% DP and ˜60% SM. The resulting mixture was concentrated under reduced pressure. To the above mixture was added CH₃NH₂ (2 M in THE, 5 mL, 10 mmol, 185.31 equiv) and irradiated with microwave radiation for 1 h at 80° C. The resulting mixture was concentrated under reduced pressure. The residue was purified by reversed-phase flash chromatography with the following conditions: Column: Sunfire prep C18 column, 30*150 mm, 5 um; Mobile Phase A: Water (0.1% FA), Mobile Phase B: ACN; Flow rate: 60 mL/min; Gradient: 15% B to 30% B in 8 min, 30% B; Wave Length: 254/220 nm; RT1 (min): 8.98; Number Of Runs: 1. This resulted in 1-[4-({5-[(4-bromo-2-fluorophenyl)amino]-4-methylpyridin-3-yl}methyl)-3-fluoropyridin-2-yl]-3-methylurea (2.1 mg, 8%) as a white solid. LCMS: (ESI, m/z): [M+1]⁺=462.05; ¹H NMR (400 MHz, DMSO-d₆) δ 9.11 (s, 1H), 8.82 (s, 1H), 8.12 (s, 2H), 7.95 (d, 1H), 7.70 (s, 1H), 7.47 (m, 1H), 7.19 (d, 1H), 6.69 (m, 1H), 6.58 (m, 1H), 4.09 (s, 2H), 2.77 (d, 3H), 2.05 (s, 3H); ¹⁹F NMR (400 MHz, DMSO-d₆) δ −125.392, δ −138.158.

### Example 13: 1-[4-({5-[(4-bromo-2-fluorophenyl)amino]-4-methylpyridin-3-yl}methyl)-3-fluoropyridin-2-yl]-3-methylurea

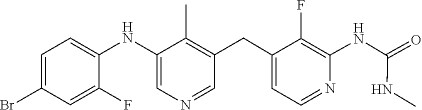 [SMILES: CNC(=O)Nc1nccc(Cc2cncc(Nc3ccc(Br)cc3F)c2C)c1F]

### Synthetic Route

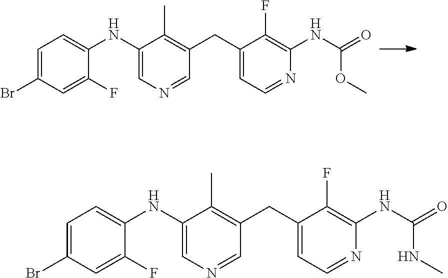 [SMILES: COC(=O)Nc1nccc(Cc2cncc(Nc3ccc(Br)cc3F)c2C)c1F ; CNC(=O)Nc1nccc(Cc2cncc(Nc3ccc(Br)cc3F)c2C)c1F]

A solution of methyl N-[4-({5-[(4-bromo-2-fluorophenyl)amino]-4-methylpyridin-3-yl}methyl)-3-fluoropyridin-2-yl]carbamate (25 mg, 0.054 mmol) and CH₃NH₂ (2 M in THE, 5 mL, 10 mmol) was irradiated with microwave radiation for 1 h at 80° C. The mixture was concentrated under reduced pressure. To the above mixture was added CH₃NH₂ (2 M in THE, 5 mL, 10 mmol) and irradiated with microwave radiation for 1 h at 80° C. The resulting mixture was concentrated under reduced pressure. The residue was purified by reversed-phase flash chromatography with the following conditions: Column: Sunfire prep C18 column, 30*150 mm, 5 um; Mobile Phase A: Water (0.1% FA), Mobile Phase B: ACN; Flow rate: 60 mL/min; Gradient: 15% B to 30% B in 8 min, 30% B; Wave Length: 254/220 nm; RT1 (min): 8.98; Number Of Runs: 1. This resulted in 1-[4-({5-[(4-bromo-2-fluorophenyl)amino]-4-methylpyridin-3-yl}methyl)-3-fluoropyridin-2-yl]-3-methylurea (2.1 mg) as a white solid. LCMS: (ESI, m/z): [M+1]⁺=462.05; ¹H NMR (400 MHz, DMSO-d₆) δ 9.11 (s, 1H), 8.82 (s, 1H), 8.12 (s, 2H), 7.95 (d, 1H), 7.70 (s, 1H), 7.47 (m, 1H), 7.19 (d, 1H), 6.69 (m, 1H), 6.58 (m, 1H), 4.09 (s, 2H), 2.77 (d, 3H), 2.05 (s, 3H); ¹⁹F NMR (400 MHz, DMSO-d₆) δ −125.392, δ −138.158.

### Example 14: 3-[(4-chloro-2-fluorophenyl)amino]-5-({3-fluoro-2-[(methylsulfamoyl)amino]pyridin-4-yl}methyl)-4-methylpyridin-1-ium-1-olate

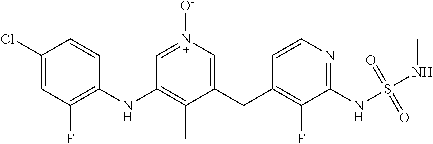 [SMILES: CNS(=O)(=O)Nc1nccc(Cc2c[n+]([O-])cc(Nc3ccc(Cl)cc3F)c2C)c1F]

### Synthetic Route

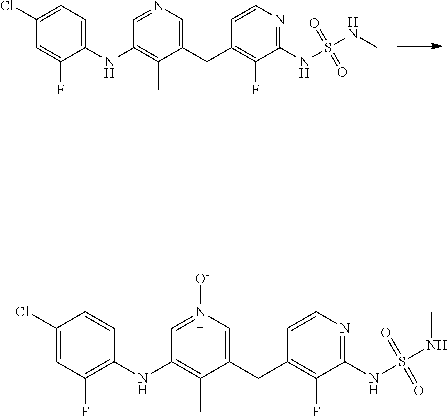

To a stirred solution of N-(4-chloro-2-fluorophenyl)-5-({3-fluoro-2-[(methylsulfamoyl)amino]pyridin-4-yl}methyl)-4-methylpyridin-3-amine (10 mg, 0.022 mmol) in DCM (1 mL) was added m-CPBA (4.18 mg, 0.024 mmol, 1.1 equiv) at 0° C. under nitrogen atmosphere. The resulting mixture was stirred for 2 h at 0° C. Desired product could be detected by LCMS. The reaction mixture was concentrated under reduced pressure. The residue was purified by reversed-phase flash chromatography with the following conditions: column, C18 silica gel; mobile phase, ACN in Water (10 mmol/L NH₄HCO₃), 5% to 60% gradient in 30 min; detector, UV 254 nm. This resulted in 3-[(4-chloro-2-fluorophenyl)amino]-5-({3-fluoro-2-[(methylsulfamoyl)amino]pyridin-4-yl}methyl)-4-methylpyridin-1-ium-1-olate (2.4 mg). LCMS: (ESI, m/z): [M+1]⁺=470.1. ¹H NMR (400 MHz, Methanol-d₄) δ 8.01 (d, J=5.1 Hz, 1H), 7.80 (d, J=1.9 Hz, 1H), 7.62 (t, J=2.0 Hz, 1H), 7.31 (m, 1H), 7.25-7.16 (m, 1H), 7.13 (t, J=8.6 Hz, 1H), 6.78 (t, J=5.1 Hz, 1H), 4.13 (s, 2H), 2.63 (s, 3H), 2.20 (s, 3H). ¹⁹F NMR (377 MHz, Methanol-d₄) δ −122.495, −142.050.

### Example 15: N-(4-chloro-2-fluorophenyl)-5-({3-fluoro-2-[(methylsulfamoyl)amino]pyridin-4-yl}oxy)-4-methylpyridin-3-amine

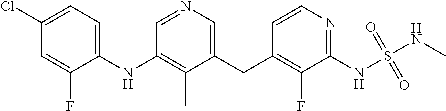 [SMILES: CNS(=O)(=O)Nc1nccc(Cc2cncc(Nc3ccc(Cl)cc3F)c2C)c1F]

### Synthetic Route

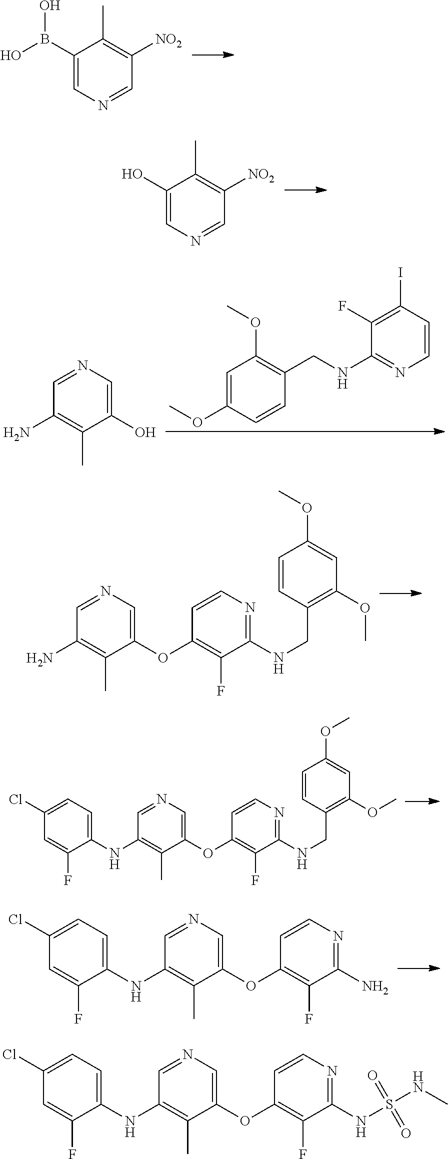

Step 1: A solution of 4-methyl-5-nitropyridin-3-ylboronic acid (1 g, 5.4 mmol, example 6) in THF (10 mL) was treated with NaOH (659.51 mg, 16.488 mmol) in H₂O (2.5 mL, 27.755 mmol) at 0° C. followed by the addition of H₂O₂ (30%) (2.56 mL, 32.976 mmol 30%) dropwise at 0° C. The resulting mixture was stirred for 1 h at 0° C. under air atmosphere. The reaction was quenched with sat. sodium hyposulfite (aq.) at 0° C. The resulting mixture was extracted with EtOAc (5×10 mL). The combined organic layers were washed with brine (10 mL), dried over anhydrous Na₂SO₄. After filtration, the filtrate was concentrated under reduced pressure. The residue was purified by silica gel column chromatography, eluted with CH₂Cl₂/MeOH (20:1) to afford 4-methyl-5-nitropyridin-3-ol (400 mg) as a white solid. LCMS: (ESI, m/z): [M+1]⁺=155.1; ¹H NMR (400 MHz, Chloroform-d) δ 8.67 (s, 1H), 8.37 (s, 1H), 2.53 (s, 3H).

Step 2: To a solution of 4-methyl-5-nitropyridin-3-ol (100 mg, 0.649 mmol) in 5 mL MeOH was added 10% Pd/C (10 mg) under nitrogen atmosphere in a 10 mL 2-necked round-bottom flask. The mixture was hydrogenated at room temperature for 1 h under hydrogen atmosphere using a hydrogen balloon, filtered through a Celite pad and concentrated under reduced pressure. This resulted in 5-amino-4-methylpyridin-3-ol (82 mg, crude) as a yellow oil. LCMS: (ESI, m/z): [M+1]⁺=125.1; ¹H NMR (400 MHz, Methanol-d₄) δ 7.49 (s, 1H), 7.40 (s, 1H), 2.03 (s, 3H).

Step 3: To a solution of 5-amino-4-methylpyridin-3-ol (240 mg, 1.933 mmol, example 1) and N-[(2,4-dimethoxyphenyl)methyl]-3-fluoro-4-iodopyridin-2-amine (900.54 mg, 2.320 mmol, 1.2 equiv) in DMSO (4 mL) were added K₃PO₄ (820.72 mg, 3.866 mmol), pyridine-2-carboxylic acid (23.80 mg, 0.193 mmol, 0.1 equiv) and CuI (18.41 mg, 0.097 mmol, 0.05 equiv). After stirring for 16 h at 80° C. under a nitrogen atmosphere. Desired product could be detected by LCMS. The resulting mixture was diluted with water (20 mL). The resulting mixture was extracted with EtOAc (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with CH₂Cl₂/MeOH (10:1) to afford 5-[(2-{[(2,4-dimethoxyphenyl)methyl]amino}-3-fluoropyridin-4-yl)oxy]-4-methylpyridin-3-amine (60 mg, 8%) as a light yellow solid. LCMS: (ESI, m/z): [M+1]⁺=385.2; ¹H NMR (300 MHz, DMSO-d₆) δ 7.85 (s, 1H), 7.60 (m, 1H), 7.53 (s, 1H), 7.07 (m, 1H), 6.93 (m, 1H), 6.54 (m, 1H), 6.44 (m, 1H), 5.86 (m, 1H), 5.44 (s, 2H), 4.45 (d, J=5.9 Hz, 2H), 3.80 (s, 3H), 3.72 (s, 3H), 1.92 (s, 3H); ¹⁹F NMR (282 MHz, DMSO-d₆) δ −165.61.

Step 4: To a solution of 5-[(2-{[(2,4-dimethoxyphenyl)methyl]amino}-3-fluoropyridin-4-yl)oxy]-4-methylpyridin-3-amine (60 mg, 0.156 mmol) and 4-chloro-2-fluoro-1-iodobenzene (48.03 mg, 0.187 mmol, 1.2 equiv) in dioxane (2 mL) were added Cs₂CO₃ (101.71 mg, 0.312 mmol), XantPhos (9.03 mg, 0.016 mmol) and Pd₂(dba)₃ (14.29 mg, 0.016 mmol). After stirring for 4 h at 80° C. under a nitrogen atmosphere. Desired product could be detected by LCMS. The resulting mixture was concentrated under reduced pressure. The residue was purified by silica gel column chromatography, eluted with PE/EA (4:1) to afford N-(4-chloro-2-fluorophenyl)-5-[(2-{[(2,4-dimethoxyphenyl)methyl]amino}-3-fluoropyridin-4-yl)oxy]-4-methylpyridin-3-amine (56 mg) as a yellow solid. LCMS: (ESI, m/z): [M+1]⁺=513.0; ¹H NMR (400 MHz, Chloroform-d) δ 8.32 (s, 1H), 8.05 (s, 1H), 7.79 (m, 1H), 7.29 (m, 1H), 7.16 (m, 1H), 7.04 (m, 1H), 6.93 m, 1H), 6.49 m, 1H), 6.45 (m, 1H), 6.02 (m, 1H), 5.47 (s, 1H), 5.15 (s, 1H), 4.64 (m, 2H), 3.86 (s, 3H), 3.81 (s, 3H), 2.18 (s, 3H); ¹⁹F NMR (376 MHz, Chloroform-d) δ −129.08, −165.21.

Step 5: To a mixture of N-(4-chloro-2-fluorophenyl)-5-[(2-{[(2,4-dimethoxyphenyl)methyl]amino}-3-fluoropyridin-4-yl)oxy]-4-methylpyridin-3-amine (56 mg, 0.109 mmol) in DCM (2 mL) was added TFA (0.5 mL) dropwise at 0° C. under nitrogen atmosphere. The resulting mixture was stirred for 1 h at 0° C. under nitrogen atmosphere. Desired product could be detected by LCMS. The resulting mixture was concentrated under reduced pressure. The residue was basified to pH 10 with saturated NaHCO₃ (aq.). The resulting mixture was extracted with EtOAc (3×10 mL). The combined organic layers were washed with brine (1×30 mL), dried over anhydrous Na₂SO₄. After filtration, the filtrate was concentrated under reduced pressure. The residue was purified by silica gel column chromatography, eluted with CH₂Cl₂/MeOH (10:1) to afford 5-[(2-amino-3-fluoropyridin-4-yl)oxy]-N-(4-chloro-2-fluorophenyl)-4-methylpyridin-3-amine (30 mg). LCMS: (ESI, m/z): [M+1]⁺=362.9; ¹H NMR (400 MHz, Chloroform-d) δ 8.36 (s, 1H), 8.08 (s, 1H), 7.74-7.68 (m, 1H), 7.17 (m, 1H), 7.05 (m, 1H), 6.95 (m, 1H), 6.10 (m, 1H), 5.53-5.44 (m, 1H), 4.88 (s, 2H), 2.18 (s, 3H); ¹⁹F NMR (377 MHz, Chloroform-d) δ −128.90, −162.99.

Step 6: To a stirred solution of 5-[(2-amino-3-fluoropyridin-4-yl)oxy]-N-(4-chloro-2-fluorophenyl)-4-methylpyridin-3-amine (45 mg, 0.124 mmol) and pyridine (98.12 mg, 1.240 mmol) in DMA (1 mL) were added N-methylsulfamoyl chloride (80.36 mg, 0.620 mmol) in DMA (1 mL) dropwise at 0° C. under nitrogen atmosphere. The resulting mixture was stirred for 1 h at room temperature under nitrogen atmosphere. Desired product could be detected by LCMS. The reaction mixture was purified by prep-HPLC with the following conditions (Column: YMC-Actus Triart C18 ExRS, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 12% B to 32% B in 9 min, 32% B; Wave Length: 254/220 nm; RT1 (min): 15.03; Number Of Runs: 0) to afford N-(4-chloro-2-fluorophenyl)-5-({3-fluoro-2-[(methylsulfamoyl)amino]pyridin-4-yl}oxy)-4-methylpyridin-3-amine (11.2 mg). LCMS: (ESI, m/z): [M+1]⁺=455.90; ¹H NMR (400 MHz, Methanol-d₄) δ 8.02 (m, 1H), 7.95 (s, 1H), 7.90 (m, 1H), 7.24 (m, 1H), 7.11 (m, 1H), 6.97 m, 1H), 6.40 (m, 1H), 2.63 (s, 3H), 2.16 (s, 3H). ¹⁹F NMR (377 MHz, Methanol-d₄) δ −125.667, −160.051.

### Example 16: N-(4-chloro-2-fluorophenyl)-5-({3-fluoro-2-[(methylsulfamoyl)amino]pyridin-4-yl}methyl)-N, 4-dimethylpyridin-3-amine

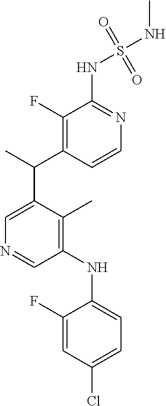 [SMILES: CNS(=O)(=O)Nc1nccc(C(C)c2cncc(Nc3ccc(Cl)cc3F)c2C)c1F]

### Synthetic Route

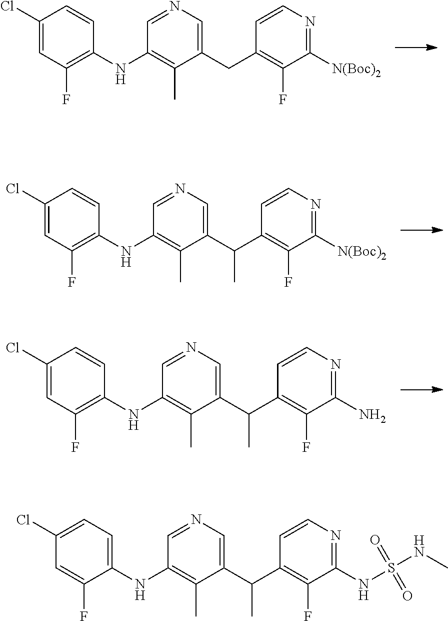

Step 1: To a stirred solution of tert-butyl N-(tert-butoxycarbonyl)-N-[4-({5-[(4-chloro-2-fluorophenyl)amino]-4-methylpyridin-3-yl}methyl)-3-fluoropyridin-2-yl]carbamate (50 mg, 0.089 mmol, example 6) in THF (1 mL) was added LiHMDS (1.3 M in THE, 0.2 mL, 0.267 mmol, 3 equiv) dropwise at −78° C. under nitrogen atmosphere. The resulting mixture was stirred for 10 min at −78° C. under nitrogen atmosphere. To the above mixture was added Mel (18.97 mg, 0.134 mmol, 1.5 equiv) in THF (1 mL) dropwise at −78° C. The resulting mixture was stirred for additional 4 h at −78° C. Desired product could be detected by LCMS. The reaction was quenched with sat. NH₄Cl (aq.) at −78° C. The resulting mixture was extracted with EA (3×10 mL). The combined organic layers were washed with brine (1×10 mL), dried over anhydrous Na₂SO₄. After filtration, the filtrate was concentrated under reduced pressure. This resulted in tert-butyl N-(tert-butoxycarbonyl)-N-[4-({5-[(4-chloro-2-fluorophenyl)(methyl)amino]-4-methylpyridin-3-yl}methyl)-3-fluoropyridin-2-yl]carbamate (60 mg). The crude product was used in the next step directly without further purification. LCMS: (ESI, m/z): [M+1]⁺=575.1

Step 2: To a stirred solution of tert-butyl N-(tert-butoxycarbonyl)-N-[4-(1-{5-[(4-chloro-2-fluorophenyl)amino]-4-methylpyridin-3-yl}ethyl)-3-fluoropyridin-2-yl]carbamate (50 mg, 0.087 mmol) in DCM (2 mL) was added TFA (0.5 mL) dropwise at 0° C. under nitrogen atmosphere. The resulting mixture was stirred for 1 h at room temperature under nitrogen atmosphere. Desired product could be detected by LCMS. The resulting mixture was concentrated under vacuum. The residue was basified to pH 10 with sat. NaHCO₃ (aq.). The resulting mixture was extracted with EA (3×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 70% gradient in 20 min; detector, UV 254 nm. This resulted in 4-(1-{5-[(4-chloro-2-fluorophenyl)amino]-4-methylpyridin-3-yl}ethyl)-3-fluoropyridin-2-amine (27 mg). LCMS: (ESI, m/z): [M+1]⁺=375.3 ¹H NMR (400 MHz, Chloroform-d) δ 8.33 (m, 2H), 7.76 (d, J=5.3 Hz, 1H), 7.13 (dd, J=10.8, 2.3 Hz, 1H), 7.01-6.93 (m, 1H), 6.69 (t, J=8.9 Hz, 1H), 6.39 (t, J=5.1 Hz, 1H), 5.42 (s, 1H), 4.73 (s, 2H), 4.61 (q, J=7.2 Hz, 1H), 2.15 (s, 3H), 1.67 (d, J=7.2 Hz, 3H). ¹⁹F NMR (377 MHz, Chloroform-d) δ −130.27, −145.86

Step 3: To a stirred solution of 5-[(2-amino-3-fluoropyridin-4-yl)methyl]-N-(4-chloro-2-fluorophenyl)-N, 4-dimethylpyridin-3-amine (10 mg, 0.027 mmol) and pyridine (21.10 mg, 0.270 mmol, 10 equiv) in DMA (0.5 mL) was added N-methylsulfamoyl chloride (17.28 mg, 0.135 mmol, 5 equiv) in DMA (0.2 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in Water (10 mmol/L NH₄HCO₃), 5% to 60% gradient in 30 min; detector, UV 254 nm. This resulted in N-(4-chloro-2-fluorophenyl)-5-({3-fluoro-2-[(methylsulfamoyl)amino]pyridin-4-yl}methyl)-N, 4-dimethylpyridin-3-amine (4.6 mg). LCMS: (ESI, m/z): [M+1]⁺=467.90 ¹H NMR (400 MHz, Methanol-d₄) δ 8.15 (s, 1H), 8.09 (s, 1H), 7.97 (d, J=5.2 Hz, 1H), 7.17 (dd, J=11.1, 2.3 Hz, 1H), 7.00 (m, 1H), 6.78 (s, 1H), 6.62 (t, J=8.9 Hz, 1H), 4.73 (q, J=7.2 Hz, 1H), 2.59 (s, 3H), 2.17 (s, 3H), 1.68 (d, J=7.1 Hz, 3H). ¹⁹F NMR (377 MHz, Methanol-d₄) δ −128.52, −142.44.

### Example 17: N-(2,4-difluorophenyl)-5-[(3-fluoro-2-methanesulfonylpyridin-4-yl)methyl]-4-methylpyridin-3-amine

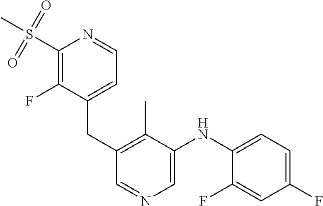 [SMILES: Cc1c(Cc2ccnc(S(C)(=O)=O)c2F)cncc1Nc1ccc(F)cc1F]

### Synthetic Route

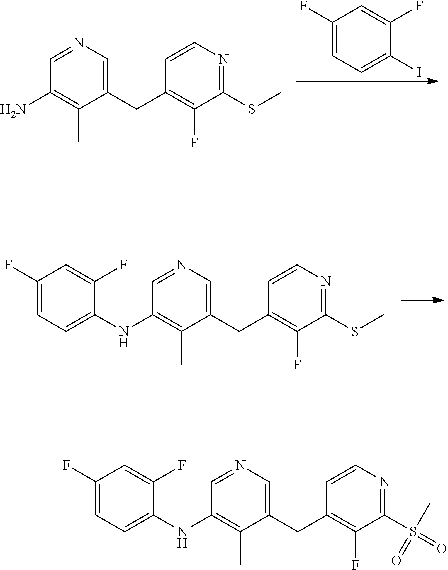 [SMILES: CSc1nccc(Cc2cncc(N)c2C)c1F ; Fc1ccc(I)c(F)c1 ; CSc1nccc(Cc2cncc(Nc3ccc(F)cc3F)c2C)c1F ; Cc1c(Cc2ccnc(S(C)(=O)=O)c2F)cncc1Nc1ccc(F)cc1F]

Step 1: A mixture of 5-{[3-fluoro-2-(methylsulfanyl)pyridin-4-yl]methyl}-4-methylpyridin-3-amine (100 mg, 0.380 mmol, example 9), 2,4-difluoro-1-iodobenzene (136.71 mg, 0.570 mmol), Cs₂CO₃ (247.46 mg, 0.760 mmol), XantPhos (21.97 mg, 0.038 mmol) and Pd₂(dba)₃ (34.77 mg, 0.038 mmol) in dioxane (10 mL) was stirred for 16 h at 100° C. under nitrogen atmosphere. Desired product could be detected by LCMS. The resulting mixture was concentrated under reduce pressure. The residue was purified by reversed-phase flash chromatography with the following conditions: column, C18 silica gel; mobile phase, ACN in water (0.1% FA), 5% to 50% gradient in 20 min; detector, UV 254 nm. This resulted in N-(2,4-difluorophenyl)-5-{[3-fluoro-2-(methylsulfanyl)pyridin-4-yl]methyl}-4-methylpyridin-3-amine (60 mg). LCMS: [M+1]⁺=376.0. ¹H NMR (400 MHz, Chloroform-d) δ 8.38-7.94 (m, 3H), 7.05-6.89 (m, 2H), 6.89-6.76 (m, 1H), 6.62 (t, J=5.2 Hz, 1H), 5.32 (s, 1H), 4.04 (s, 2H), 2.59 (s, 3H), 2.18 (s, 3H). ¹⁹F NMR (376 MHz, Chloroform-d) δ −117.03, −125.17, −126.97.

Step 2: To a stirred solution of N-(2,4-difluorophenyl)-5-{[3-fluoro-2-(methylsulfanyl)pyridin-4-yl]methyl}-4-methylpyridin-3-amine (50 mg, 0.133 mmol) in acetone (2 mL), H₂O (2 mL) and MeOH (0.2 mL) was added oxone (89.58 mg, 0.532 mmol) in portions at 0° C. The resulting mixture was stirred for 16 h at room temperature. Desired product could be detected by LCMS. The resulting mixture was concentrated under reduced pressure. The residue was purified by Prep-HPLC with the following conditions: Column: YMC-Actus Triart C18 ExRS, 30*150 mm, 5 um; Mobile Phase A: water (10 mmol/L NH₄HCO₃), Mobile Phase B: ACN; Flow rate: 60 mL/min; Gradient: 25% B to 45% B in 9 min, 45% B; Wave Length: 254/220 nm; RT1 (min): 11.25; Number Of Runs: 3. This resulted in N-(2,4-difluorophenyl)-5-[(3-fluoro-2-methanesulfonylpyridin-4-yl)methyl]-4-methylpyridin-3-amine (13.6 mg). LCMS: (ESI, m/z): [M+1]⁺=408.10. ¹H NMR (400 MHz, Methanol-d₄) δ 8.39 (d, J=4.8 Hz, 1H), 8.01 (s, 1H), 7.94 (s, 1H), 7.38 (t, J=5.1 Hz, 1H), 7.06-6.85 (m, 3H), 4.28 (s, 2H), 3.37 (s, 3H), 2.17 (s, 3H). ¹⁹F NMR (377 MHz, Methanol-d₄) δ −120.10, −123.51, −125.85.

### Example 18: 4-[[5-(4-chloro-2-fluoro-anilino)-3-pyridyl]methyl]-3-fluoro-N-(methylsulfamoyl)pyridin-2-amine

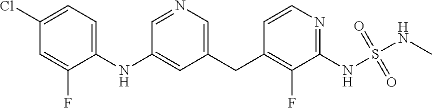 [SMILES: CNS(=O)(=O)Nc1nccc(Cc2cncc(Nc3ccc(Cl)cc3F)c2)c1F]

### Synthetic Route

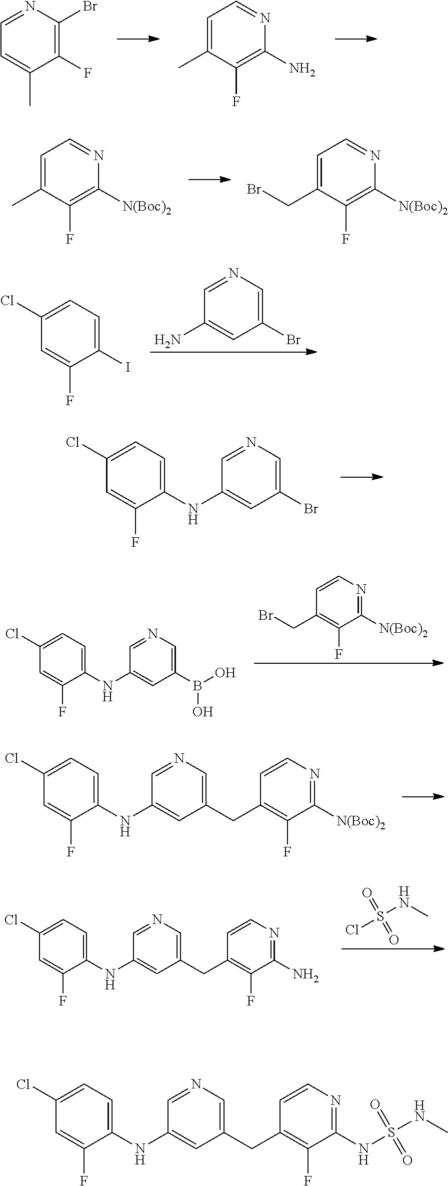

### Intermediate

Step 1: A solution of 2-bromo-3-fluoro-4-methyl-pyridine (20 g, 105.26 mmol) in NH₄OH (200 mL) and ethylene glycol (250 mL) were added Cu₂O (753.06 mg, 5.26 mmol, 537.90 μL), K₂CO₃ (2.91 g, 21.05 mmol) and N′,N′-dimethylethane-1,2-diamine (927.85 mg, 10.53 mmol, 1.15 mL). The mixture was stirred at 80° C. for 12 hours in a 1000 mL of autoclave. After cooling to room temperature, the reaction mixture was poured into water (50 mL) and extracted with DCM (50 mL×3). The combined organic layers were washed with brine (50 mL), dried over anhydrous Na₂SO₄, filtered and concentrated. The residue was purified by flash chromatography on silica gel (ethyl acetate in petroleum ether=0-25%) to 3-fluoro-4-methyl-pyridin-2-amine (10 g, 79.28 mmol). LCMS Rₜ=0.232 min in 1.5 min chromatography, 5-95CD, ESI calcd. for C₂₂H₂₃FNO₅ [M+H]⁺127.1, found 126.9.

Step 2: To a solution of 3-fluoro-4-methyl-pyridin-2-amine (10 g, 79.28 mmol) in DCM (100 mL) were added Boc₂O (38.07 g, 174.42 mmol, 40.07 mL), DMAP (968.58 mg, 7.93 mmol), TEA (24.07 g, 237.85 mmol, 33.11 mL). The mixture was stirred at 25° C. for 12 hr. Water (80 mL) was added and the mixture were extracted with EtOAc (50 mL×2). The organic layer was dried over anhydrous Na₂SO₄, filtered and concentrated. The crude was purified by flash column chromatography on silica gel (EtOAc in petroleum ether=0-30%) to give tert-butyl N-tert-butoxycarbonyl-N-(3-fluoro-4-methyl-2-pyridyl) carbamate (19 g, 58.22 mmol) as yellow oil. ¹H NMR (400 MHz, CDCl₃) δ=8.14 (d, J=4.8 Hz, 1H), 7.13 (t, J=4.8 Hz, 1H), 2.33 (s, 3H), 1.42 (s, 18H). ¹⁹F NMR (376.5 MHz, CDCl₃) δ=−131.767 ppm.

Step 3: To a solution of tert-butyl N-tert-butoxycarbonyl-N-(3-fluoro-4-methyl-2-pyridyl) carbamate (17 g, 52.09 mmol) in DCE (170 mL) were added AIBN (1.71 g, 10.42 mmol) and NBS (27.81 g, 156.27 mmol). The mixture was stirred at 85° C. for 4 hr. The reaction was concentrated. Water (100 mL) was added and the aqueous layer was extracted with DCM (2×100 mL). The combined organic layers were dried over anhydrous Na₂SO₄, filtered and concentrated.

To a solution of the crude in MeCN (200 mL) were added DIPEA (13.46 g, 104.18 mmol, 18.15 mL) and 1-ethoxyphosphonoyloxyethane (719.38 mg, 5.21 mmol, 672.31 μL). The mixture was stirred at 25° C. for 1 hr. The mixture was concentrated. The residue was poured into water (200 mL) and extracted with EtOAc (100 mL×3). The combined organic layers were washed with brine (200 mL), dried over anhydrous Na₂SO₄, concentrated under reduced pressure. The crude was purified by flash chromatography on silica gel (Ethyl acetate in Petroleum ether=0 to 27%) to give tert-butyl N-[4-(bromomethyl)-3-fluoro-2-pyridyl]-N-tert-butoxycarbonyl-carbamate (17.6 g, 23.89 mmol).

Tert-butyl N-[4-(bromomethyl)-3-fluoro-2-pyridyl]-N-tert-butoxycarbonyl-carbamate (7 g, 9.50 mmol, 55% purity) of the product was purified by Prep-HPLC (column: Xtimate C18 150×40 mm×10 um; mobile phase: [water(NH₃H₂O+NH₄HCO₃)−ACN]; B %: 49%-79%, 8 min) to give tert-butyl N-[4-(bromomethyl)-3-fluoro-2-pyridyl]-N-tert-butoxycarbonyl-carbamate (3 g, 7.40 mmol). ¹H NMR (400 MHz, CDCl₃) δ=8.27 (d, J=5.2 Hz, 1H), 7.32 (t, J=5.2 Hz, 1H), 4.45 (s, 2H), 1.42 (s, 18H). ¹⁹F NMR (376.5 MHz, CDCl₃) δ=−130.835 ppm.

### Target:

Step 1: To a solution of 5-bromopyridin-3-amine (4.8 g, 27.74 mmol) in 1,4-dioxane (50 mL) were added Pd(OAc)₂ (622.87 mg, 2.77 mmol), 4-chloro-2-fluoro-1-iodo-benzene (7.11 g, 27.74 mmol), Cs₂CO₃ (18.08 g, 55.49 mmol), and Xantphos (3.21 g, 5.55 mmol). The mixture was stirred at 80° C. for 2 hr. The mixture were poured into H₂O (50 mL) and EtOAc (50 mL×3). The combined organic layers were washed with brine (50 mL), dried over Na₂SO₄, filtered and concentrated under reduced pressure. The crude was purified by flash chromatography on silica gel (Ethyl acetate in Petroleum ether=0 to 20%) to give 5-bromo-N-(4-chloro-2-fluoro-phenyl)pyridin-3-amine (7.6 g, 25.20 mmol). ¹H NMR (400 MHz, CDCl₃) δ=8.29 (d, J=2.8 Hz, 1H), 8.25 (d, J=1.6 Hz, 1H), 7.49 (t, J=2.0 Hz, 1H), 7.24-7.16 (m, 2H), 7.13-7.06 (m, 1H), 5.81 (br s, 1H). ¹⁹F NMR (376.5 MHz, CDCl₃) δ=−126.453 ppm.

Step 2: To a solution of 5-bromo-N-(4-chloro-2-fluoro-phenyl)pyridin-3-amine (1 g, 3.32 mmol) in dioxane (10 mL) were added 4,4,5,5-tetramethyl-2-(4,4,5,5-tetramethyl-1,3,2-dioxaborolan-2-yl)-1,3,2-dioxaborolane (1.68 g, 6.63 mmol), Pd(dppf)Cl₂ (121.33 mg, 165.81 μmol) and KOAc (976.40 mg, 9.95 mmol). The mixture was stirred at 100° C. for 4 hr. The mixture was concentrated. [5-(4-chloro-2-fluoro-anilino)-3-pyridyl]boronic acid (883.67 mg, 3.32 mmol) as solid was used next step without purification. LCMS Rt=0.679 min in 1.5 min chromatography, 5-95CD, ESI calcd. for C₁₁H₁₀BClFN₂O₂ [M+H]⁺267.0, found 266.9.

Step 3: To a solution of [5-(4-chloro-2-fluoro-anilino)-3-pyridyl]boronic acid (883.67 mg, 3.32 mmol) and tert-butyl N-[4-(bromomethyl)-3-fluoro-2-pyridyl]-N-tert-butoxycarbonyl-carbamate (2.44 g, 3.32 mmol) in dioxane (10 mL) and H₂O (1 mL) were added Pd(dppf)Cl₂ (121.33 mg, 165.81 μmol) and K₂CO₃ (1.37 g, 9.95 mmol). The mixture was stirred at 100° C. for 2 hr. The reaction mixture was concentrated. The residue was poured into water (10 mL) and extracted with EtOAc (10 mL×3). The combined organic layers were washed with brine (10 mL), dried over anhydrous Na₂SO₄, concentrated under reduced pressure. The crude was purified by flash chromatography on silica gel (Ethyl acetate in Petroleum ether=0 to 27%) to give tert-butyl N-tert-butoxycarbonyl-N-[4-[[5-(4-chloro-2-fluoro-anilino)-3-pyridyl]methyl]-3-fluoro-2-pyridyl]carbamate (490 mg, 895.81 μmol). LCMS Rt=0.869 min in 1.5 min chromatography, 5-95CD, ESI calcd. For C₂₇H₃₀ClF₂N₄O₄ [M+H]⁺547.2, found 547.0.

Step 4: To a solution of tert-butyl N-tert-butoxycarbonyl-N-[4-[[5-(4-chloro-2-fluoro-anilino)-3-pyridyl]methyl]-3-fluoro-2-pyridyl]carbamate (490 mg, 895.81 μmol) in MeOH (5 mL) was added HCl/MeOH (4 M, 5.00 mL). The mixture was stirred at 25° C. for 2 hr. The residue was poured into water (10 mL) and extracted with DCM (10 mL×3). Saturated NaHCO₃ solution was added dropwise to adjust pH=7 at 0° C. The combined organic layers were washed with brine (10 mL), dried over anhydrous Na₂SO₄, concentrated under reduced pressure. The crude was purified by flash chromatography on silica gel (Ethyl acetate in Petroleum ether=0 to 27%) to give 4-[[5-(4-chloro-2-fluoro-anilino)-3-pyridyl]methyl]-3-fluoro-pyridin-2-amine (150 mg, 432.57 μmol). ¹H NMR (400 MHz, CDCl₃) δ=8.34 (d, J=2.4 Hz, 1H), 8.15 (d, J=1.4 Hz, 1H), 7.77 (d, J=5.2 Hz, 1H), 7.22-7.15 (m, 3H), 7.11-7.04 (m, 1H), 6.47 (t, J=5.2 Hz, 1H), 5.89 (br s, 1H), 4.92 (br s, 2H), 3.95 (s, 2H). ¹⁹F NMR (376.5 MHz, CDCl₃) δ=−127.266, −145.251 ppm.

Step 5: To a solution of 4-[[5-(4-chloro-2-fluoro-anilino)-3-pyridyl]methyl]-3-fluoro-pyridin-2-amine (30 mg, 86.51 μmol) in MeCN (1 mL) and DMA (1 mL) were added Py (68.43 mg, 865.15 μmol, 69.83 μL) and N-methylsulfamoyl chloride (112.09 mg, 865.15 μmol). The mixture was stirred at 25° C. for 2 hr. The mixture was concentrated. The crude was purified by Prep-HPLC (column: Xtimate C18 150×40 mm×10 um; mobile phase: [column: Welch Xtimate C₁₈ 150×30 mm×5 um; mobile phase: [water(NH₃H₂O+ACN]; B %: 22%-52%, 7 min) to give 4-[[5-(4-chloro-2-fluoro-anilino)-3-pyridyl]methyl]-3-fluoro-N-(methylsulfamoyl)pyridin-2-amine (9 mg, 20.46 μmol). ¹H NMR (400 MHz, DMSO-d₆) δ=8.31 (br s, 1H), 8.15 (d, J=2.4 Hz, 1H), 8.02-7.95 (m, 2H), 7.43 (dd, J=2.4, 11.2 Hz, 1H), 7.26 (t, J=8.8 Hz, 1H), 7.20-7.15 (m, 2H), 7.03-6.86 (m, 1H), 3.97 (s, 2H), 2.49 (s, 3H). ¹⁹F NMR (376.5 MHz, DMSO-d₆) δ=−122.147, −139.025 ppm. LCMS Rₜ=0.667 min in 1.5 min chromatography, 5-95AB, ESI calcd. for C₁₈H₁₇ClF₂N₅O₂S [M+H]⁺440.1, found 440.0.

### Example 19: 3-fluoro-4-({5-[(3-fluoropyridin-2-yl)oxy]-4-methylpyridin-3-yl}methyl)pyridin-2-amine

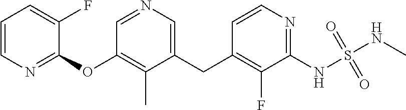

### Synthetic Route

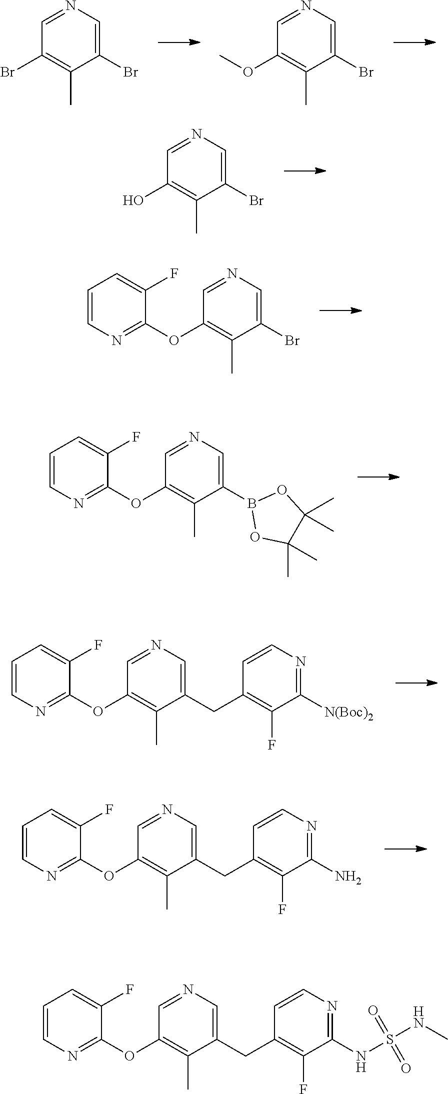

Step 1: To a stirred solution of 3,5-dibromo-4-methylpyridine (30 g, 119.560 mmol) in DMF (300 mL) was added NaOMe (6.46 g, 119.560 mmol) in portions at room temperature under nitrogen atmosphere. The resulting mixture was stirred for 16 h at 80° C. under nitrogen atmosphere. The reaction mixture was diluted with water (2 L). The resulting mixture was extracted with EA (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EA (6:1) to afford 3-bromo-5-methoxy-4-methylpyridine (13.6 g). LCMS: (ESI, m/z): [M+1]⁺=201.9. ¹H NMR (400 MHz, Chloroform-d) δ 8.33 (s, 1H), 8.09 (s, 1H), 3.92 (s, 3H), 2.32 (s, 3H).

Step 2: To a stirred solution of 3-bromo-5-methoxy-4-methylpyridine (10 g, 49.49 mmol) in DCM (100 mL) was added BBr₃ (1 M in DCM, 99 mL, 99 mmol) dropwise at −78° C. under nitrogen atmosphere. The resulting mixture was stirred for 16 h at room temperature under nitrogen atmosphere. Desired product could be detected by LCMS. The reaction was quenched with MeOH at −78° C. The resulting mixture was concentrated under reduced pressure. The residue was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in 5-bromo-4-methylpyridin-3-ol (6.2 g). LCMS: (ESI, m/z): [M+1]⁺=187.8. ¹H NMR (400 MHz, DMSO-d₆) δ 8.62 (s, 1H), 8.27 (s, 1H), 2.38 (s, 3H).

Step 3: To a stirred mixture of 5-bromo-4-methylpyridin-3-ol (2.1 g, 11.16 mmol) and 2,3-difluoropyridine (2.57 g, 22.33 mmol) in DMSO (21 mL) was added Cs₂CO₃ (14.56 g, 44.67 mmol) at room temperature under nitrogen atmosphere. The resulting mixture was stirred for 16 h at 60° C. under nitrogen atmosphere. Desired product could be detected by LCMS. The reaction mixture was filtered, the filtrate was concentrated under reduce pressure, the residue was purified by reversed-phase flash chromatography with the following conditions: column, C18 silica gel; mobile phase, ACN in water (10 mmol/L NH₄HCO₃), 5% to 50% gradient in 20 min; detector, UV 254 nm. This resulted in 3-bromo-5-[(3-fluoropyridin-2-yl)oxy]-4-methylpyridine (1.8 g). LCMS: (ESI, m/z): [M+1]⁺=283.0. ¹H NMR (400 MHz, Chloroform-d) δ 8.57 (s, 1H), 8.33 (s, 1H), 7.86 (m, 1H), 7.55-7.49 (m, 1H), 7.07-7.01 (m, 1H), 2.31 (s, 3H).

Step 4: To a stirred mixture of 3-bromo-5-[(3-fluoropyridin-2-yl)oxy]-4-methylpyridine (400 mg, 1.41 mmol) and bis(pinacolato)diboron (430.56 mg, 1.696 mmol, 1.2 equiv) in dioxane (10 mL) were added AcOK (277.34 mg, 2.86 mmol) and Pd(PPh₃)₂Cl₂ (99.17 mg, 0.141 mmol, 0.1 equiv). The resulting mixture was stirred for 16 h at 80° C. under nitrogen atmosphere. Desired product could be detected by LCMS. The reaction mixture was concentrated under reduced pressure. This resulted in 3-[(3-fluoropyridin-2-yl)oxy]-4-methyl-5-(4,4,5,5-tetramethyl-1,3,2-dioxaborolan-2-yl)pyridine (400 mg). LCM. S: (ESI, m/z): [M+1]⁺=331.2

Step 5: To a stirred mixture of 3-[(3-fluoropyridin-2-yl)oxy]-4-methyl-5-(4,4,5,5-tetramethyl-1,3,2-dioxaborolan-2-yl)pyridine (488.82 mg, 1.480 mmol) and tert-butyl N-[4-(bromomethyl)-3-fluoropyridin-2-yl]-N-(tert-butoxycarbonyl) carbamate (300 mg, 0.740 mmol, 0.5 equiv) in dioxane (6 mL) and H₂O (0.6 mL) were added K₂CO₃ (614 mg, 4.44 mmol, 3 equiv) and Pd(dppf)Cl₂ (108.2 mg, 0.148 mmol, 0.1 equiv). The resulting mixture was stirred for 2 h at 80° C. under nitrogen atmosphere. Desired product could be detected by LCMS. The resulting mixture was concentrated under vacuum. The residue was purified by reversed-phase flash chromatography with the following conditions: column, C18 silica gel; mobile phase, ACN in water (0.1% FA), 10% to 95% gradient in 20 min; detector, UV 254 nm. This resulted in tert-butyl N-(tert-butoxycarbonyl)-N-[3-fluoro-4-({5-[(3-fluoropyridin-2-yl)oxy]-4-methylpyridin-3-yl}methyl)pyridin-2-yl]carbamate (170 mg). LCMS: (ESI, m/z): [M+1]⁺=529.2. ¹H NMR (400 MHz, Chloroform-d) δ 8.44 (s, 1H), 8.30 (s, 1H), 8.22 (d, J=4.7 Hz, 1H), 7.87 (dd, J=4.9, 1.5 Hz, 1H), 7.53 (ddd, J=9.5, 7.8, 1.5 Hz, 1H), 7.06 (ddd, J=8.0, 4.8, 3.2 Hz, 1H), 6.93 (t, J=4.9 Hz, 1H), 4.15 (s, 2H), 2.16 (s, 3H), 1.42 (s, 18H).

Step 6: To a stirred solution of tert-butyl N-(tert-butoxycarbonyl)-N-[3-fluoro-4-({5-[(3-fluoropyridin-2-yl)oxy]-4-methylpyridin-3-yl}methyl)pyridin-2-yl]carbamate (170 mg, 0.322 mmol) in DCM (4 mL) were added TFA (1 mL) dropwise at 0° C. The resulting mixture was stirred for 1 h at room temperature. Desired product could be detected by LCMS. The resulting mixture was concentrated under reduced pressure. The residue was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in 3-fluoro-4-({5-[(3-fluoropyridin-2-yl)oxy]-4-methylpyridin-3-yl}methyl)pyridin-2-amine (60 mg). LCMS: (ESI, m/z): [M+1]⁺=328.9. ¹H NMR (400 MHz, Chloroform-d) δ 8.36 (m, 2H), 7.86 (m, 1H), 7.78 (s, 1H), 7.50 (m, 1H), 7.01 (m, 1H), 6.31 (t, J=5.1 Hz, 1H), 4.67 (s, 2H), 4.02 (s, 2H), 2.11 (s, 3H). ¹⁹F NMR (376 MHz, Chloroform-d) −137.69, −145.09.

### Example 20: N-(4-chloro-2-fluoro-phenyl)-5-[(2-fluoro-4-methylsulfonyl-phenyl)methyl]-4-methyl-pyridin-3-amine

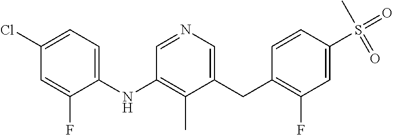 [SMILES: Cc1c(Cc2ccc(S(C)(=O)=O)cc2F)cncc1Nc1ccc(Cl)cc1F]

### Synthetic Route

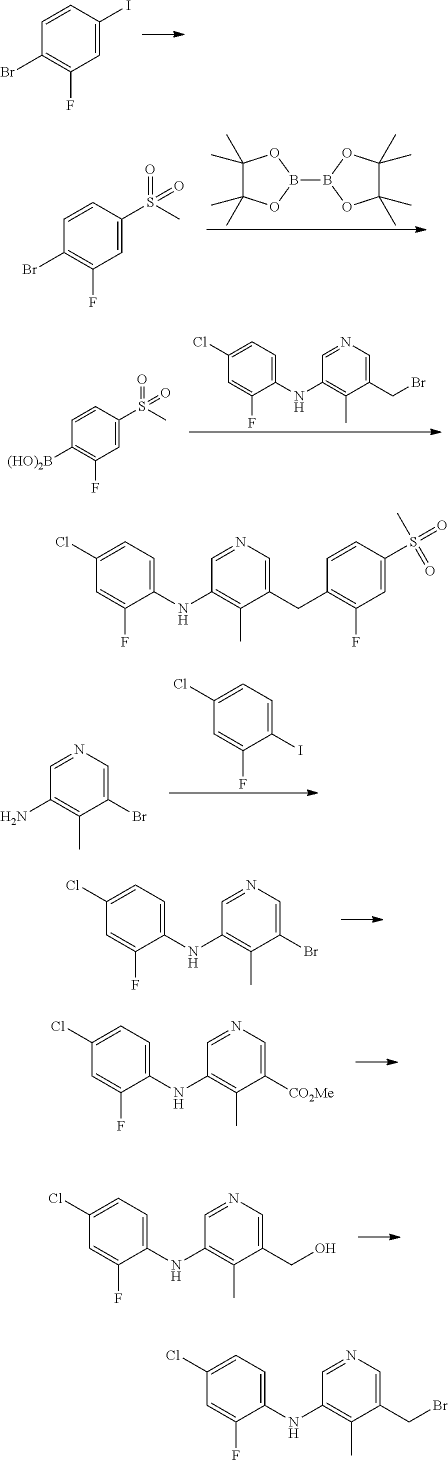

Step 1: To a solution of 1-bromo-2-fluoro-4-iodo-benzene (3 g, 9.97 mmol) and sodium methane sulfinate (1.22 g, 11.96 mmol) in DMSO (25 mL) were added Cu(OAc)₂ (90.55 mg, 498.51 μmol), DMEDA (87.89 mg, 997.03 μmol, 107.31 μL) and K₂CO₃ (2.76 g, 19.94 mmol). The mixture was stirred at 110° C. for 9 h. H₂O (30 mL) was added to the mixture. The aqueous phase was extracted with EtOAc (30 mL×3). The combined organic phase was washed with brine (30 mL×3), dried over anhydrous Na₂SO₄, filtered and concentrated. The crude was purified by flash chromatography on silica gel (ethyl acetate in petroleum ether=0-20%) to give 1-bromo-2-fluoro-4-methylsulfonyl-benzene (1.16 g, 4.58 mmol). ¹H NMR (400 MHz, CDCl₃) δ=7.80 (dd, J=6.4, 8.4 Hz, 1H), 7.70 (dd, J=2.0, 7.6 Hz, 1H), 7.63 (dd, J=2.0, 8.4 Hz, 1H), 3.07 (s, 3H). ¹⁹F NMR (376.5 MHz, CDCl₃) δ=−102.196 ppm.

Step 2: To a solution of 1-bromo-2-fluoro-4-methylsulfonyl-benzene (1 g, 3.95 mmol) in dioxane (10 mL) were added 4,4,5,5-tetramethyl-2-(4,4,5,5-tetramethyl-1,3,2-dioxaborolan-2-yl)-1,3,2-dioxaborolane (3.01 g, 11.85 mmol) and KOAc (1.16 g, 11.85 mmol). The mixture was degassed and purged with N₂ for 3 times. Then Pd(dppf)Cl₂ (289.11 mg, 395.12 μmol) was added to the mixture, degassed and purged with N₂ for 3 times. The mixture was stirred at 100° C. for 3 h under N₂ atmosphere. The reaction mixture was concentrated and purified by flash chromatography on silica gel (ethyl acetate in petroleum ether=0-10%) to give (2-fluoro-4-methylsulfonyl-phenyl) boronic acid (650 mg, 2.98 mmol). ¹H NMR (400 MHz, CDCl₃) δ=7.82-7.77 (m, 1H), 7.72-7.69 (m, 1H), 7.64-7.61 (m, 1H), 3.07 (s, 3H), 1.58 (s, 2H). ¹⁹F NMR (376.5 MHz, CDCl₃) δ=−102.207 ppm.

Step 3: To a solution of (2-fluoro-4-methylsulfonyl-phenyl) boronic acid (148.82 mg, 682.65 μmol) and 5-(bromomethyl)-N-(4-chloro-2-fluoro-phenyl)-4-methyl-pyridin-3-amine (150 mg, 455.10 μmol) in toluene (3 mL) and EtOH (1.5 mL) was added Na₂CO₃ (192.94 mg, 1.82 mmol). The mixture was degassed and purged with N₂ for 3 times. Then Pd(PPh₃)₄ (52.59 mg, 45.51 μmol) was added to the mixture, degassed and purged with N₂ for 3 times. The mixture was stirred at 80° C. for 2 h. The reaction mixture was concentrated and purified by flash chromatography on silica gel (Ethyl acetate in Petroleum ether=0-50%) and prep-HPLC (column: YMC Triart C₁₈ 70*250 mm*7 μm; mobile phase: [water(NH₄HCO₃)−ACN]; B %: 30%-60%, 15 min) to afford N-(4-chloro-2-fluoro-phenyl)-5-[(2-fluoro-4-methylsulfonyl-phenyl)methyl]-4-methyl-pyridin-3-amine (5 mg, 11.82 μmol). ¹H NMR (400 MHz, CDCl₃) δ=8.41 (br s, 1H), 8.17 (br s, 1H), 7.67 (t, J=8.8 Hz, 2H), 7.22-7.10 (m, 2H), 6.98 (d, J=8.8 Hz, 1H), 6.75 (t, J=8.4 Hz, 1H), 5.42 (s, 1H), 4.12 (s, 2H), 3.07 (s, 3H), 2.14 (s, 3H). ¹⁹F NMR (376.5 MHz, CDCl₃) δ=−112.684, −130.327 ppm. LCMS Rₜ=0.735 min in 1.5 min chromatography, 5-95AB, ESI calcd. for C₂₀H₁₈ClF₂N₂O₂S [M+H]⁺423.1, found 422.9.

Step 1 to intermediate bromide: To a solution of 5-bromo-4-methyl-pyridin-3-amine (5 g, 26.73 mmol) in 1,4-dioxane (100 mL) were added 4-chloro-2-fluoro-1-iodo-benzene (6.86 g, 26.73 mmol), Pd(OAc)₂ (600.17 mg, 2.67 mmol), Xantphos (3.09 g, 5.35 mmol) and Cs₂CO₃ (17.42 g, 53.47 mmol). The mixture was stirred at 100° C. for 12 hr. Water (80 mL) was added and the mixture were extracted with EtOAc (50 mL×2). The organic layer was dried over anhydrous Na₂SO₄, filtered and concentrated. The crude was purified by flash column chromatography on silica gel (EtOAc in petroleum ether=0-10%) to give 5-bromo-N-(4-chloro-2-fluoro-phenyl)-4-methyl-pyridin-3-amine (6.4 g, 20.28 mmol). ¹H NMR (400 MHz, DMSO-d₆) δ=8.37 (s, 1H), 8.08 (s, 1H), 7.90 (s, 1H), 7.40 (dd, J=2.0, 11.6 Hz, 1H), 7.13 (d, J=8.4 Hz, 1H), 6.83 (t, J=8.8 Hz, 1H), 2.27 (s, 3H). ¹⁹F NMR (376.5 MHz, DMSO-d₆) δ=−123.680.

Step 2: To a solution of 5-bromo-N-(4-chloro-2-fluoro-phenyl)-4-methyl-pyridin-3-amine (5 g, 15.84 mmol) in MeOH (70 mL) were added TEA (12.83 g, 126.76 mmol, 17.64 mL) and Pd(dppf)Cl₂ (2.32 g, 3.17 mmol). The mixture was stirred at 60° C. for 12 hr under CO (50 Psi). Water (50 mL) was added and the mixture were extracted with EtOAc (50 ml×2). The organic layer was dried over anhydrous Na₂SO₄, filtered and concentrated. The crude was purified by flash column chromatography on silica gel (EtOAc in petroleum ether=0-20%) to give methyl 5-(4-chloro-2-fluoro-anilino)-4-methyl-pyridine-3-carboxylate (2.8 g, 9.50 mmol). ¹H NMR (400 MHz, DMSO-d₆) δ=8.59 (s, 1H), 8.29 (s, 1H), 7.85 (s, 1H), 7.40 (dd, J=2.4, 11.6 Hz, 1H), 6.74 (d, J=8.8 Hz, 1H), 3.87 (s, 3H), 2.35 (s, 3H). ¹⁹F NMR (376.5 MHz, DMSO-d₆) δ=−124.380.

Step 3: To a solution of LiAlH₄ (695.44 mg, 18.32 mmol) in THF (60 mL) in three-neck bottle under N₂ at 0° C. was added methyl 5-(4-chloro-2-fluoro-anilino)-4-methyl-pyridine-3-carboxylate (2.7 g, 9.16 mmol). The mixture was stirred at 25° C. for 3 hr under N₂. Water (3 mL) and 15% NaOH (3 mL) and H₂O (9 mL) were added successively to the mixture at 0° C. and the mixture was stirred at 25° C. for 30 min. Then THF (80 mL) was added. The resulting mixture was filtered, and the filter cake was washed with EtOAc (30 mL×3). Then the filtrate was concentrated to afford [5-(4-chloro-2-fluoro-anilino)-4-methyl-3-pyridyl]methanol (2.4 g, 9.00 mmol), which was used directly for the next step without purification. 1H NMR (400 MHz, DMSO-d₆) δ=8.24 (s, 1H), 8.13 (s, 1H), 7.68 (s, 1H), 7.36 (dd, J=2.4, 11.2 Hz, 1H), 7.05 (d, J=8.4 Hz, 1H), 6.56 (t, J=9.2 Hz, 1H), 5.22 (t, J=5.2 Hz, 1H), 4.55 (d, J=5.2 Hz, 2H), 2.12 (s, 3H). ¹⁹F NMR (376.5 MHz, DMSO-d₆) δ=−126.121.

Step 4: To a solution of [5-(4-chloro-2-fluoro-anilino)-4-methyl-3-pyridyl]methanol (2.3 g, 8.62 mmol) in DCM (25 mL) was added PBr₃ (7.00 g, 25.87 mmol, 2.43 mL). The mixture was stirred at 25° C. for 2 hr. Water (40 mL) was added and the mixture were extracted with DCM (30 mL×2). The organic layer was dried over anhydrous Na₂SO₄, filtered and concentrated to afford 5-(bromomethyl)-N-(4-chloro-2-fluoro-phenyl)-4-methyl-pyridin-3-amine (2.6 g, 7.89 mmol, 91.47% yield) as a yellow solid, which was used directly for the next step without purification. ¹H NMR (400 MHz, CDCl₃) δ=8.39 (s, 1H), 8.31 (s, 1H), 7.14 (dd, J=2.0, 10.8 Hz, 1H), 6.99 (d, J=8.8 Hz, 1H), 6.75 (d, J=9.2 Hz, 1H), 5.46 (s, 1H), 4.53 (s, 2H), 2.31 (s, 3H). ¹⁹F NMR (376.5 MHz, CDCl₃) δ=−130.065.

### Example 21: N-(4-chloro-2-fluoro-phenyl)-6-[[3-fluoro-2-(methylsulfamoylamino)-4-pyridyl]methyl]pyrazin-2-amine

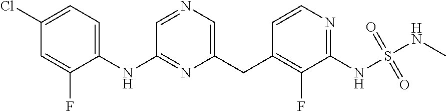 [SMILES: CNS(=O)(=O)Nc1nccc(Cc2cncc(Nc3ccc(Cl)cc3F)n2)c1F]

### Synthetic Route

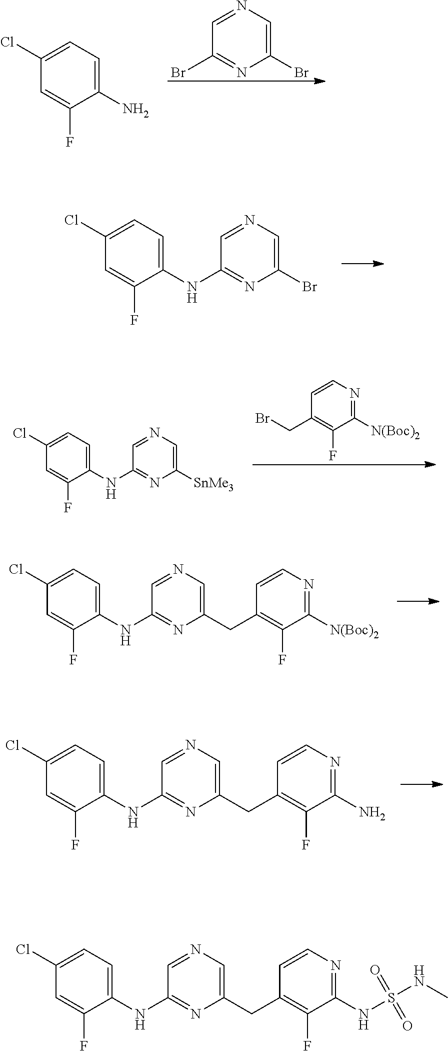

Step 1: To a solution of 4-chloro-2-fluoro-aniline (3.06 g, 21.02 mmol) and 2,6-dibromopyrazine (5 g, 121.02 mmol) in toluene (100 mL) were added dicyclohexyl-[2-(2,4,6-triisopropylphenyl)phenyl]phosphane (501.00 mg, 1.05 mmol), NaOt-Bu (3.03 g, 31.53 mmol) and Pd(PPh₃)₄ (2.43 g, 2.10 mmol). The mixture was stirred at 80° C. for 2 hr. The reaction mixture was poured into water (20 mL) and extracted with EtOAc (20 mL×3). The combined organic layers were washed with brine (50 mL), dried over anhydrous Na₂SO₄, filtered and concentrated. The residue was purified by flash chromatography on silica gel (ethyl acetate in petroleum ether=0-25%) to give 6-bromo-N-(4-chloro-2-fluoro-phenyl) pyrazin-2-amine (4.46 g, 14.74 mmol). ¹H NMR (400 MHz, DMSO-d₆) δ=9.65 (br s, 1H), 8.33 (s, 1H), 8.12 (s, 1H), 7.99 (t, J=8.8 Hz, 1H), 7.52 (dd, J=2.4, 11.2 Hz, 1H), 7.31 (d, J=8.8 Hz, 1H). ¹⁹F NMR (376.5 MHz, DMSO-d₆) δ=−121.267 ppm. LCMS Rₜ=1.017 min 1.5 min chromatography, 5-95AB, ESI calcd. for C₁₀H₇BrClFN₃ [M+H]⁺303.9 found 303.8.

Step 2: To a solution of 6-bromo-N-(4-chloro-2-fluoro-phenyl) pyrazin-2-amine (500 mg, 1.65 mmol) and trimethyl(trimethylstannyl) stannane (590 mg, 1.80 mmol, 373.42 μL) in dioxane (5 mL) was added Pd(PPh₃)₄ (190.98 mg, 165.27 μmol). The mixture was stirred at 90° C. for 2 h. The mixture was used for the next step directly. N-(4-chloro-2-fluoro-phenyl)-6-trimethylstannyl-pyrazin-2-amine (638.68 mg, 1.65 mmol). LCMS Rₜ=1.036 min 1.5 min chromatography, 5-95AB, ESI calcd. for C₁₃H₁₆ClFN₃Sn [M+H]⁺386.0 found 385.7.

Step 3: To a solution of N-(4-chloro-2-fluoro-phenyl)-6-trimethylstannyl-pyrazin-2-amine (538 mg, 1.39 mmol) in dioxane (10 mL) was added tert-butyl N-[4-(bromomethyl)-3-fluoro-2-pyridyl]-N-tert-butoxycarbonyl-carbamate (846.30 mg, 2.09 mmol, from example 18), DPPF (308.72 mg, 556.88 μmol) Pd(OAc)₂ (62.51 mg, 278.44 μmol) and CsF (845.92 mg, 5.57 mmol, 205.32 μL). The mixture was stirred at 90° C. for 2 h. H₂O (30 mL) was added. The residue was extracted with EtOAc (50 mL×3). The combined organic layers were washed with brine (30 mL), dried over anhydrous Na₂SO₄, filtered and concentrated. The crude product was purified by flash chromatography on silica gel (EtOAc in petroleum ether=0-35%) to give tert-butyl N-tert-butoxycarbonyl-N-[4-[[6-(4-chloro-2-fluoro-anilino) pyrazin-2-yl]methyl]-3-fluoro-2-pyridyl]carbamate (400 mg, 729.95 μmol). ¹H NMR (400 MHz, CDCl₃) δ=8.25 (d, J=5.2 Hz, 1H), 8.11-8.05 (m, 2H), 7.99 (s, 1H), 7.17-7.06 (m, 2H), 7.00-6.66 (m, 1H), 4.13 (s, 2H), 1.38 (s, 18H). ¹⁹F NMR (376.5 MHz, CDCl₃) δ=−131.407

Step 4: To a solution of tert-butyl N-tert-butoxycarbonyl-N-[4-[[6-(4-chloro-2-fluoro-anilino) pyrazin-2-yl]methyl]-3-fluoro-2-pyridyl]carbamate (400 mg, 729.95 μmol) in MeOH (1 mL) was added HCl/MeOH (4 M, 6.96 mL, 27.83 mmol). The mixture was stirred at 25° C. for 12 h. NH₃/MeOH (10 mL×3) was added. The mixture was concentrated. The crude product was purified by Prep-HPLC (column: Boston Prime C18 150×30 mm×5 um; mobile phase: [water(NH₃H₂O+NH₄HCO₃)−ACN]; B %: 53%-53%, 7 min) to give 6-[(2-amino-3-fluoro-4-pyridyl)methyl]-N-(4-chloro-2-fluoro-phenyl) pyrazin-2-amine (120 mg, 345.08 μmol). ¹H NMR (400 MHz, CDCl₃) δ=8.14-7.93 (m, 3H), 7.80 (d, J=5.2 Hz, 1H), 7.17-7.00 (m, 2H), 6.71-6.51 (m, 2H), 4.68 (s, 2H), 4.04 (s, 2H). ¹⁹F NMR (376.5 MHz, CDCl₃) δ=−128.701, −145.392.

Step 5: To a solution of 6-[(2-amino-3-fluoro-4-pyridyl)methyl]-N-(4-chloro-2-fluoro-phenyl) pyrazin-2-amine (35 mg, 100.65 μmol) in DMA (0.6 mL) was added pyridine (47.77 mg, 603.88 μmol, 48.74 μL). Then N-methylsulfamoyl chloride (130.40 mg, 1.01 mmol) in CH₃CN (0.6 mL) was added. The mixture was stirred at 28° C. for 1 h. The mixture was concentrated. The crude product was purified by Prep-HPLC (column: Welch Xtimate C18 150×30 mm×5 μm; mobile phase: [water(NH₃H₂O+NH₄HCO₃)−ACN]; B %: 25%-55%, 7 min) to give N-(4-chloro-2-fluoro-phenyl)-6-[[3-fluoro-2-(methylsulfamoylamino)-4-pyridyl]methyl]pyrazin-2-amine (16.1 mg, 36.52 umol). ¹H NMR (400 MHz, DMSO-d₆) δ=10.39 (s, 1H), 9.30 (s, 1H), 8.29 (s, 1H), 8.05-7.99 (m, 3H), 7.41 (d, J=14.0 Hz, 1H), 7.13 (d, J=8.8 Hz, 1H), 7.08-6.98 (m, 2H), 4.09 (s, 2H), 2.48 (s, 3H). ¹⁹F NMR (376.5 MHz, DMSO-d₆) δ=−123.604, −139.020 ppm. LCMS Rₜ=0.813 min 1.5 min chromatography, 5-95AB, ESI calcd. for C₁₇H₁₆ClF₂N₆O_{2S} [M+H]⁺441.1, found 441.0.

### Example 22: 5-({3-fluoro-2-[(methylsulfamoyl)amino]pyridin-4-yl}methyl)-N-(2-fluoro-4-methoxyphenyl)-4-methylpyridin-3-amine

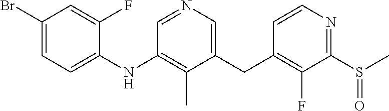 [SMILES: Cc1c(Cc2ccnc(S(C)=O)c2F)cncc1Nc1ccc(Br)cc1F]

### Synthetic Route

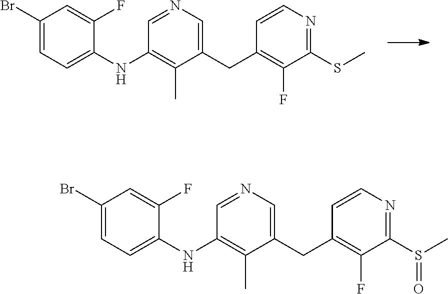 [SMILES: CSc1nccc(Cc2cncc(Nc3ccc(Br)cc3F)c2C)c1F ; Cc1c(Cc2ccnc(S(C)=O)c2F)cncc1Nc1ccc(Br)cc1F]

To a stirred solution of N-(4-bromo-2-fluorophenyl)-5-{[3-fluoro-2-(methylsulfanyl)pyridin-4-yl]methyl}-4-methylpyridin-3-amine (56 mg, 0.128 mmol, example 9) in 2 mL acetone/H₂O/MeOH/(v/v/v=10/10/1) was added oxone (43.17 mg, 0.256 mmol) in portions at 0° C. under nitrogen atmosphere. The resulting mixture was stirred for 16 h at room temperature. Desired product could be detected by LCMS. The reaction mixture was concentrated under reduced pressure. The residue was purified by Prep-HPLC with the following conditions: Column: YMC-Actus Triart C18 ExRS, 30*150 mm, 5 um; Mobile Phase A: Water (10 mmol/L NH₄HCO₃), Mobile Phase B: ACN; Flow rate: 60 mL/min; Gradient: 23% B to 45% B in 9 min, 45% B; Wave Length: 254/220 nm; RT1 (min): 11.02; Number Of Runs: 3. This resulted in N-(4-bromo-2-fluorophenyl)-5-[(3-fluoro-2-methanesulfonylpyridin-4-yl)methyl]-4-methylpyridin-3-amine (16 mg). LCMS: (ESI, m/z): [M+1]⁺=451.951H NMR (400 MHz, Methanol-d₄) δ 8.49 (d, J=4.8 Hz, 1H), 8.15-8.11 (m, 2H), 7.34-7.30 (m, 2H), 7.16 (ddd, J=8.6, 2.3, 1.2 Hz, 1H), 6.67 (t, J=8.8 Hz, 1H), 4.27 (s, 2H), 2.98 (s, 3H), 2.16 (s, 3H). ¹⁹F NMR (377 MHz, Methanol-d₄) δ −128.03, −129.66.

### Example 23

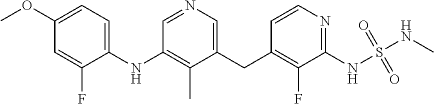

### Synthetic Route

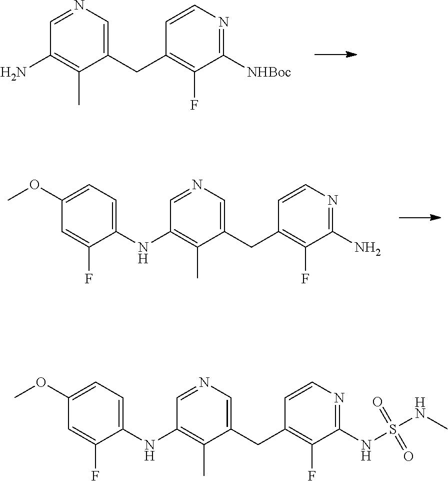

Step 1: To a solution of tert-butyl N-{4-[(5-amino-4-methylpyridin-3-yl)methyl]-3-fluoropyridin-2-yl}carbamate (100 mg, 0.301 mmol) and 1-bromo-2-fluoro-4-methoxybenzene (61.68 mg, 0.301 mmol, example 6) in dioxane (5 mL) were added Cs₂CO₃ (294.08 mg, 0.903 mmol, 3 equiv), EPhos (16.09 mg, 0.030 mmol, 0.1 equiv) and EPhos Pd G4 (27.64 mg, 0.030 mmol, 0.1 equiv). After stirring for 16 h at 80° C. under a nitrogen atmosphere, the resulting mixture was concentrated under reduced pressure. The residue was purified by silica gel column chromatography, eluted with CH₂Cl₂/MeOH (10:1) to afford 3-fluoro-4-({5-[(2-fluoro-4-methoxyphenyl)amino]-4-methylpyridin-3-yl}methyl)pyridin-2-amine (PH-NEST-M-68-2, 24 mg). LCMS: (ESI, m/z): [M+1]⁺=357.11H NMR (400 MHz, Chloroform-d) δ 8.14 (s, 1H), 8.03 (s, 1H), 7.68 (d, J=5.3 Hz, 1H), 6.98 (t, J=9.1 Hz, 1H), 6.79-6.50 (m, 2H), 6.24 (t, J=5.1 Hz, 1H), 5.19 (s, 1H), 4.87 (s, 2H), 3.96 (s, 2H), 3.78 (s, 3H), 2.12 (s, 3H). ¹⁹F NMR (376 MHz, Chloroform-d) δ −125.96, −145.29.

Step 2: To a stirred solution of 3-fluoro-4-({5-[(2-fluoro-4-methoxyphenyl)amino]-4-methylpyridin-3-yl}methyl)pyridin-2-amine (18 mg, 0.051 mmol) and pyridine (39.95 mg, 0.510 mmol, 10 equiv) in DMA (0.3 mL) were added N-methylsulfamoyl chloride (32.72 mg, 0.255 mmol, 5 equiv) in DMA (0.3 mL) dropwise at 0° C. under nitrogen atmosphere. The resulting mixture was stirred for 2 h at room temperature under nitrogen atmosphere. Desired product could be detected by LCMS. The reaction mixture was purified by Prep-HPLC with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 27% B to 42% B in 10 min, 42% B; Wavelength: 254/220 nm; RT1 (min): 9.437; Number Of Runs: 0). This resulted in (19.1 mg). LCMS: (ESI, m/z): [M+1]⁺=450.15. ¹H NMR (400 MHz, Methanol-d₄) δ 7.95 (d, J=5.2 Hz, 1H), 7.86 (s, 1H), 7.72 (d, J=1.8 Hz, 1H), 7.02 (t, J=9.0 Hz, 1H), 6.79 (dd, J=12.5, 2.8 Hz, 1H), 6.75-6.71 (m, 1H), 6.65 (t, J=5.0 Hz, 1H), 4.11 (s, 2H), 3.79 (s, 3H), 2.63 (s, 3H), 2.17 (s, 3H). ¹⁹F NMR (377 MHz, Methanol-d₄) δ −123.53, −142.44.

### Example 24

### ({3-fluoro-4-[(5-methoxy-4-methylpyridin-3-yl)methyl]pyridin-2-yl}sulfamoyl)(methyl)amine

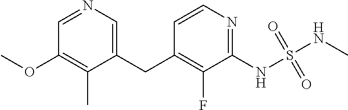

### Synthetic Route

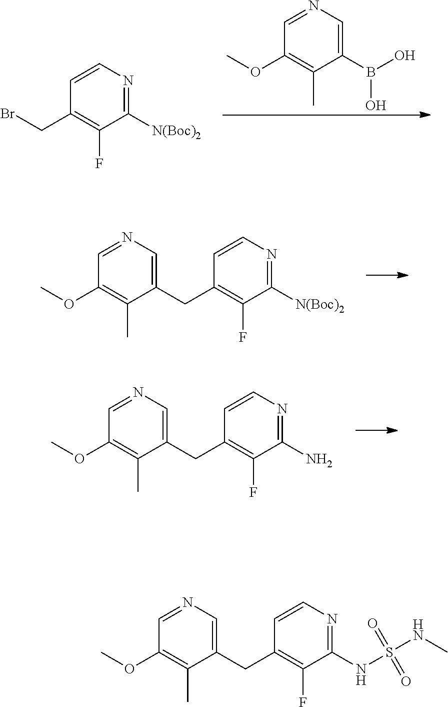

Step 1: To a stirred mixture of tert-butyl N-[4-(bromomethyl)-3-fluoropyridin-2-yl]-N-(tert-butoxycarbonyl) carbamate (200 mg, 0.494 mmol) and 5-methoxy-4-methylpyridin-3-ylboronic acid (98.88 mg, 0.593 mmol, 1.2 equiv) in 1,4-dioxane were added K₂CO₃ (206.11 mg, 1.482 mmol, 3 equiv) and Pd(dppf)Cl₂ (36.11 mg, 0.049 mmol, 0.1 equiv) at room temperature under nitrogen atmosphere. The resulting mixture was stirred for 2 h at 80° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, MeCN in Water (0.1% FA), 30% to 50% gradient in 15 min; detector, UV 254/220 nm to afford tert-butyl N-(tert-butoxycarbonyl)-N-{3-fluoro-4-[(5-methoxy-4-methylpyridin-3-yl)methyl]pyridin-2-yl}carbamate (92 mg,) as a brown oil. LCMS: [M+1]⁺=448.1

Step 2: To a stirred solution of tert-butyl N-(tert-butoxycarbonyl)-N-{3-fluoro-4-[(5-methoxy-4-methylpyridin-3-yl)methyl]pyridin-2-yl}carbamate (92 mg, 0.206 mmol) in DCM (4 mL) was added TFA (1 mL) dropwise at 0° C. under nitrogen atmosphere. The resulting mixture was stirred for 1 h at room temperature under nitrogen atmosphere. Desired product could be detected by LCMS. The resulting mixture was concentrated under reduced pressure. The residue was basified to pH 10 with saturated NaHCO₃ (aq.). The resulting mixture was extracted with EA (3×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 40% to 70% gradient in 20 min; detector, UV 254/220 nm to afford 3-fluoro-4-[(5-methoxy-4-methylpyridin-3-yl)methyl]pyridin-2-amine (60 mg) as a white solid. LCMS: (ESI, m/z): [M+1]⁺=248.1

Step 3: To a stirred mixture of 3-fluoro-4-[(5-methoxy-4-methylpyridin-3-yl)methyl]pyridin-2-amine (53 mg, 0.214 mmol) in DMA were added pyridine (84.77 mg, 1.070 mmol, 5 equiv) and N-methylsulfamoyl chloride (27.77 mg, 0.214 mmol) dropwise at 0° C. under nitrogen atmosphere. The resulting mixture was stirred for 1 h at room temperature under nitrogen atmosphere. Desired product could be detected by LCMS. The residue was purified by reversed-phase flash chromatography with the following conditions: column, C18 silica gel; mobile phase, MeCN in Water (10 mmol/L NH₄HCO₃), 30% to 60% gradient in 20 min; detector, UV 254/220 nm to afford (20 mg) as a white solid. LCMS: (ESI, m/z): [M+1]⁺=341. ¹H NMR (400 MHz, Methanol-d₄) δ 8.30-7.68 (m, 3H), 6.65 (m, 1H), 4.11 (s, 2H), 3.94 (s, 3H), 2.62 (s, 3H), 2.15 (s, 3H); ¹⁹F NMR (377 MHz, Methanol-d₄) δ −142.455.

### Example 25: N-(4-chloro-2-fluoro-phenyl)-5-[[3-methoxy-4-(methylsulfamoylamino)phenyl]methyl]-4-methyl-pyridin-3-amine

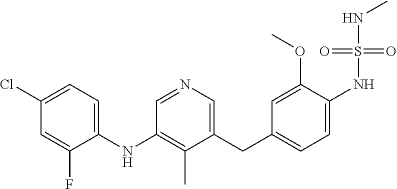 [SMILES: CNS(=O)(=O)Nc1ccc(Cc2cncc(Nc3ccc(Cl)cc3F)c2C)cc1OC]

### Synthetic Route

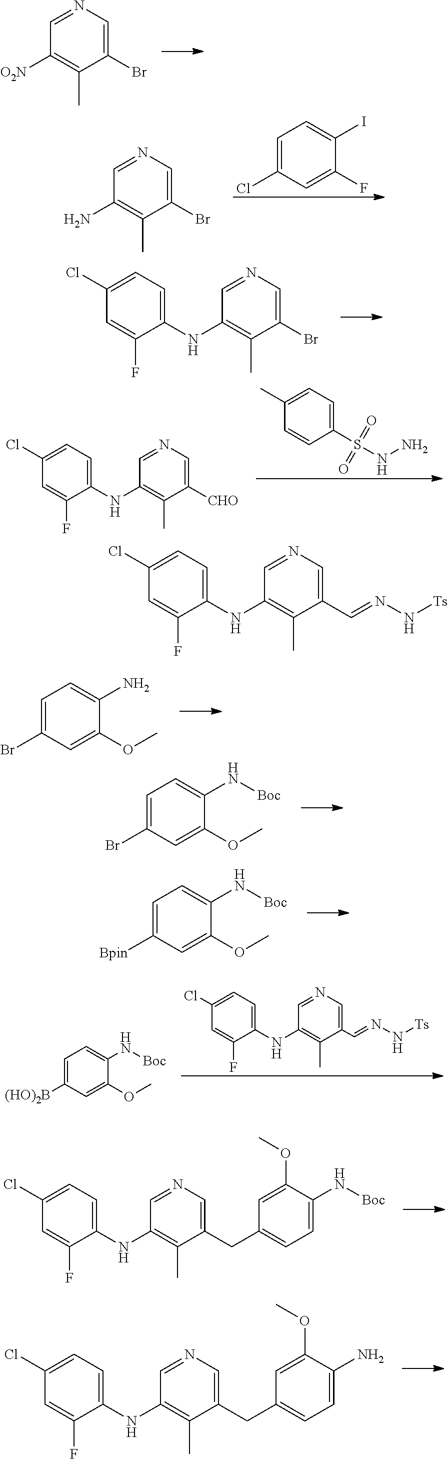

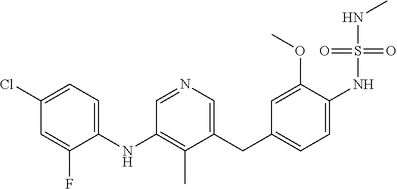 [SMILES: CNS(=O)(=O)Nc1ccc(Cc2cncc(Nc3ccc(Cl)cc3F)c2C)cc1OC]

### Intermediate for Step 4 (Target Synthesis)

Step 1: To a solution of 3-bromo-4-methyl-5-nitro-pyridine (50 g, 230.39 mmol) in EtOH (1250 mL) and H₂O (250 mL) were added Fe (128.66 g, 2.30 mol) and NH₄Cl (36.97 g, 691.17 mmol). The mixture was stirred at 80° C. for 12 hr. After cooling to room temperature, water (200 mL) was added to the mixture and the aqueous layer was extracted with EtOAc (100 mL×3). The combined organic phase was washed with brine (100 mL), dried over anhydrous Na₂SO₄, filtered and concentrated under reduced pressure to give 5-bromo-4-methyl-pyridin-3-amine (34 g, 181.78 mmol), was used next step without purification. LCMS Rₜ=0.286 min 1.5 min chromatography, 5-95AB, ESI calcd. for C₆H₈BrN₂ [M+H]⁺189.0 found 188.7. ¹H NMR (400 MHz, CDCl₃) δ=8.11 (s, 1H), 7.92 (s, 1H), 3.95-3.01 (m, 2H), 2.26 (s, 3H).

Step 2: To a solution of 4-chloro-2-fluoro-1-iodo-benzene (6.86 g, 26.73 mmol) in dioxane (100 mL) were added 5-bromo-4-methyl-pyridin-3-amine (5 g, 26.73 mmol) and Cs₂CO₃ (17.42 g, 53.47 mmol) under N₂, then Pd(OAc)₂ (600.17 mg, 2.67 mmol) and Xantphos (3.09 g, 5.35 mmol) was added. The mixture was stirred at 100° C. for 10 hours under N₂. The mixture was cooled to 25° C. The mixture was filtered and the filtrate was concentrated under reduced pressure. The reaction mixture was poured into water (20 mL) and extracted with EtOAc (20 mL×3). The combined organic layers were washed with brine (50 mL), dried over anhydrous Na₂SO₄, filtered and concentrated. The residue was purified by flash chromatography on silica gel (ethyl acetate in petroleum ether=0-25%) to 5-bromo-N-(4-chloro-2-fluoro-phenyl)-4-methyl-pyridin-3-amine (6.2 g, 19.65 mmol). ¹H NMR (400 MHz, CDCl₃) δ=8.43 (s, 1H), 8.31 (s, 1H), 7.14 (dd, J=2.4, 10.8 Hz, 1H), 6.99 (d, J=8.8 Hz, 1H), 6.77 (t, J=8.8 Hz, 1H), 5.55 (s, 1H), 2.35 (s, 3H). ¹⁹F NMR (376.5 MHz, CDCl₃) δ=−129.53 ppm.

Step 3: Toluene (30 mL) in a 100 mL 3-neckedflask was cooled down to −60° C. n-BuLi (2.5 M, 5.02 mL) was mixed with the toluene. A solution of 5-bromo-N-(4-chloro-2-fluoro-phenyl)-4-methyl-pyridin-3-amine (1.8 g, 5.70 mmol) in toluene (10 mL) was added. The mixture was stirred at −60° C. for 30 min, then THF (10 mL) was added slowly. The mixture was aged for 15 min, then DMF (500.31 mg, 6.84 mmol, 526.64 μL) was added at −60° C. The mixture was stirred at −60° C. for 30 min. Water (100 mL) was added and the aqueous layer was extracted with EtOAc (100 mL×3). The combined organic layer was washed with brine (100 mL), dried over anhydrous Na₂SO₄, filtered and concentrated. The mixture was purified by flash chromatography on silica gel (EtOAc in petroleum ether=0-30%) to afford 5-(4-chloro-2-fluoro-anilino)-4-methyl-pyridine-3-carbaldehyde (1.4 g, 5.29 mmol). ¹H NMR (400 MHz, CDCl₃) δ=10.34 (s, 1H), 8.70 (s, 1H), 8.60 (s, 1H), 7.22-7.09 (m, 1H), 7.07-6.94 (m, 1H), 6.88-6.71 (m, 1H), 5.52 (br s, 1H), 2.59 (s, 3H).

Step 4: To a solution of 5-(4-chloro-2-fluoro-anilino)-4-methyl-pyridine-3-carbaldehyde (860 mg, 3.25 mmol) in MeOH (8 mL) was added 4-methylbenzenesulfonohydrazide (605.10 mg, 3.25 mmol). The mixture was stirred at 60° C. for 2 hr. The mixture was concentrated. The crude product was triturated from MeOH (3 mL) to give N-[(E)-[5-(4-chloro-2-fluoro-anilino)-4-methyl-3-pyridyl]methyleneamino]-4-methyl-benzenesulfonamide (840 mg, 1.94 mmol). ¹H NMR (400 MHz, CDCl₃) δ=8.51 (s, 1H), 8.39 (s, 1H), 8.33-8.09 (m, 1H), 8.01 (s, 1H), 7.88 (d, J=8.4 Hz, 2H), 7.34 (d, J=8.0 Hz, 2H), 7.14 (dd, J=2.4, 10.8 Hz, 1H), 6.97 (d, J=8.8 Hz, 1H), 6.67 (t, J=8.8 Hz, 1H), 5.45 (s, 1H), 2.43 (s, 3H), 2.31 (s, 3H). ¹⁹F NMR (376.5 MHz, CDCl₃) δ=−130.010 ppm.

### Target Route:

Step 1: To a solution of tert-butoxycarbonyl tert-butyl carbonate (2.38 g, 10.89 mmol, 2.50 mL) in THF (20 mL) were added DIPEA (3.84 g, 29.70 mmol, 5.17 mL) and 4-bromo-2-methoxy-aniline (2 g, 9.90 mmol). The mixture was stirred at 25° C. for 4 hr. The reaction mixture was concentrated. The residue was poured into water (10 mL) and extracted with EtOAc (10 mL×3). The combined organic layers were washed with brine (10 mL), dried over anhydrous Na₂SO₄, concentrated under reduced pressure. The crude was purified by flash chromatography on silica gel (Ethyl acetate in Petroleum ether=0 to 27%) to give tert-butyl N-(4-bromo-2-methoxy-phenyl) carbamate (1.8 g, 5.96 mmol). ¹H NMR (400 MHz, CDCl₃) δ=7.96 (d, J=8.0 Hz, 1H), 7.06 (dd, J=2.0, 8.4 Hz, 1H), 7.00 (br s, 1H), 6.95 (d, J=2.0 Hz, 1H), 3.85 (s, 3H), 1.52 (s, 9H).

Step 2: To a solution of tert-butyl N-(4-bromo-2-methoxy-phenyl) carbamate (500 mg, 1.65 mmol) in dioxane (5 mL) were added 4,4,5,5-tetramethyl-2-(4,4,5,5-tetramethyl-1,3,2-dioxaborolan-2-yl)-1,3,2-dioxaborolane (840.40 mg, 3.31 mmol), KOAc (487.20 mg, 4.96 mmol) and Pd(dppf)Cl₂ (60.54 mg, 82.74 umol). The mixture was stirred at 80° C. for 12 hr. The mixture was filtered. The filtrate concentrated under reduced pressure. The crude product was purified by flash column chromatography on silica gel (EtOAc in petroleum ether=0-20%) to afford tert-butyl N-[2-methoxy-4-(4,4,5,5-tetramethyl-1,3,2-dioxaborolan-2-yl)phenyl]carbamate (570 mg, 1.63 mmol). ¹H NMR (400 MHz, CDCl₃) δ=8.10 (d, J=7.6 Hz, 1H), 7.43 (d, J=8.4 Hz, 1H), 7.25-7.20 (m, 2H), 3.91 (s, 3H), 1.52 (s, 9H), 1.34 (s, 12H).

Step 3: To a solution of tert-butyl N-[2-methoxy-4-(4,4,5,5-tetramethyl-1,3,2-dioxaborolan-2-yl)phenyl]carbamate (570 mg, 1.63 mmol) in acetone (20 mL) was added NaIO₄ (1.75 g, 8.16 mmol, 452.21 μL) and KOAc (1 M, 8.16 mL). The mixture was stirred at 20° C. for 16 hr. The resulting solution was diluted water (20 mL) and quenched with Saturated Na₂SO₃ solution until KI test paper turn to white. The mixture extracted with ethyl acetate (20 mL×3). The organic layers were combined and dried over sodium sulfate. The solids were filtered out and the solution was concentrated. The crude product was purified by flash column chromatography on silica gel (MeOH in DCM=0-10%) to afford [4-(tert-butoxycarbonylamino)-3-methoxy-phenyl]boronic acid (330 mg, 1.24 mmol). ¹H NMR (400 MHz, CDCl₃) δ=8.17 (d, J=8.0 Hz, 1H), 7.77 (d, J=7.6 Hz, 1H), 7.57 (br s, 1H), 7.22-7.19 (m, 1H), 3.94 (s, 3H), 1.49 (s, 9H).

Step 4: To a solution of N-[(E)-[5-(4-chloro-2-fluoro-anilino)-4-methyl-3-pyridyl]methyleneamino]-4-methyl-benzenesulfonamide (120 mg, 277.20 μmol) and [4-(tert-butoxycarbonylamino)-3-methoxy-phenyl]boronic acid (185.09 mg, 693.00 μmol) in dioxane (2 mL) was added K₂CO₃ (114.94 mg, 831.60 μmol). The mixture was stirred at 25° C. for 1 hr. The mixture was concentrated. The residue was poured into DCM (2 mL) and filtered. The filtrate concentrated under reduced pressure to give tert-butyl N-[4-[[5-(4-chloro-2-fluoro-anilino)-4-methyl-3-pyridyl]methyl]-2-methoxy-phenyl]carbamate (130.83 mg, 277.21 μmol) was used next step without purification LCMS Rₜ=0.821 min 1.5 min chromatography, 5-95AB, ESI calcd., for C₂₅H₂₈ClFN₃O₃ [M+H]⁺472.2 found 472.1.

Step 5: To a solution of tert-butyl N-[4-[[5-(4-chloro-2-fluoro-anilino)-4-methyl-3-pyridyl]methyl]-2-methoxy-phenyl]carbamate (130.83 mg, 277.21 μmol) in MeOH (2 mL) was added HCl/MeOH (4 M, 2 mL). The mixture was stirred at 25° C. for 2 hr. The residue was poured into water (10 mL) and extracted with DCM (10 mL×3). The mixture was slowly dropped sat. NaHCO₃ (20 ml) to adjust pH=7. The combined organic layers were washed with brine (10 mL), dried over anhydrous Na₂SO₄, concentrated under reduced pressure to give a residue. The crude was purified by flash chromatography on silica gel (Methanol in Dichloromethane=0 to 10%) to give 5-[(4-amino-3-methoxy-phenyl)methyl]-N-(4-chloro-2-fluoro-phenyl)-4-methyl-pyridin-3-amine (90 mg, 242.04 μmol). LCMS Rₜ=0.683 min 1.5 min chromatography, 5-95AB, ESI calcd. for C₂₀H₂₀ClFN3O [M+H]⁺372.1 found 372.0.

Step 6: To a solution of 5-[(4-amino-3-methoxy-phenyl)methyl]-N-(4-chloro-2-fluoro-phenyl)-4-methyl-pyridin-3-amine (50 mg, 134.47 μmol) in DCM (1 mL) was added TEA (40.82 mg, 403.40 μmol, 56.15 μL) and N-methylsulfamoyl chloride (52.27 mg, 403.40 umol). The mixture was stirred at 25° C. for 1 hr. The mixture was concentrated. The crude was purified by flash chromatography on silica gel (Ethyl acetate in Petroleum ether=0 to 50%) and Prep-HPLC (column: Welch Xtimate C18 150×30 mm×5 um; mobile phase: [water(NH₃H₂O+NH₄HCO₃)−ACN]; B %: 45%-75%, 8 min) to give N-(4-chloro-2-fluoro-phenyl)-5-[[3-methoxy-4-(methylsulfamoylamino)phenyl]methyl]-4-methyl-pyridin-3-amine (19 mg, 40.87 μmol). ¹H NMR (400 MHz, CDCl₃) δ=8.6 (s, 1H), 8.16 (s, 1H), 7.38 (d, J=8.0 Hz, 1H), 7.12 (dd, J=2.4, 10.8 Hz, 1H), 6.96 (d, J=8.8 Hz, 1H), 6.86 (br s, 1H), 6.78-6.61 (m, 3H), 5.44 (br s, 1H), 4.59-4.48 (m, 1H), 3.99 (s, 2H), 3.82 (s, 3H), 2.68 (d, J=5.2 Hz, 3H), 2.13 (s, 3H). ¹⁹F NMR (376.5 MHz, CDCl₃) δ=−130.456 ppm. LCMS Rₜ=0.699 min 1.5

### Example 26: 4-[[5-(4-chloro-2-fluoro-anilino)-4-fluoro-3-pyridyl]methyl]-3-fluoro-N-(methylsulfamoyl)pyridin-2-amine

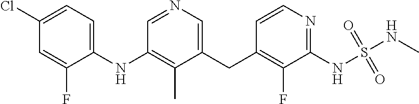 [SMILES: CNS(=O)(=O)Nc1nccc(Cc2cncc(Nc3ccc(Cl)cc3F)c2C)c1F]

### Route

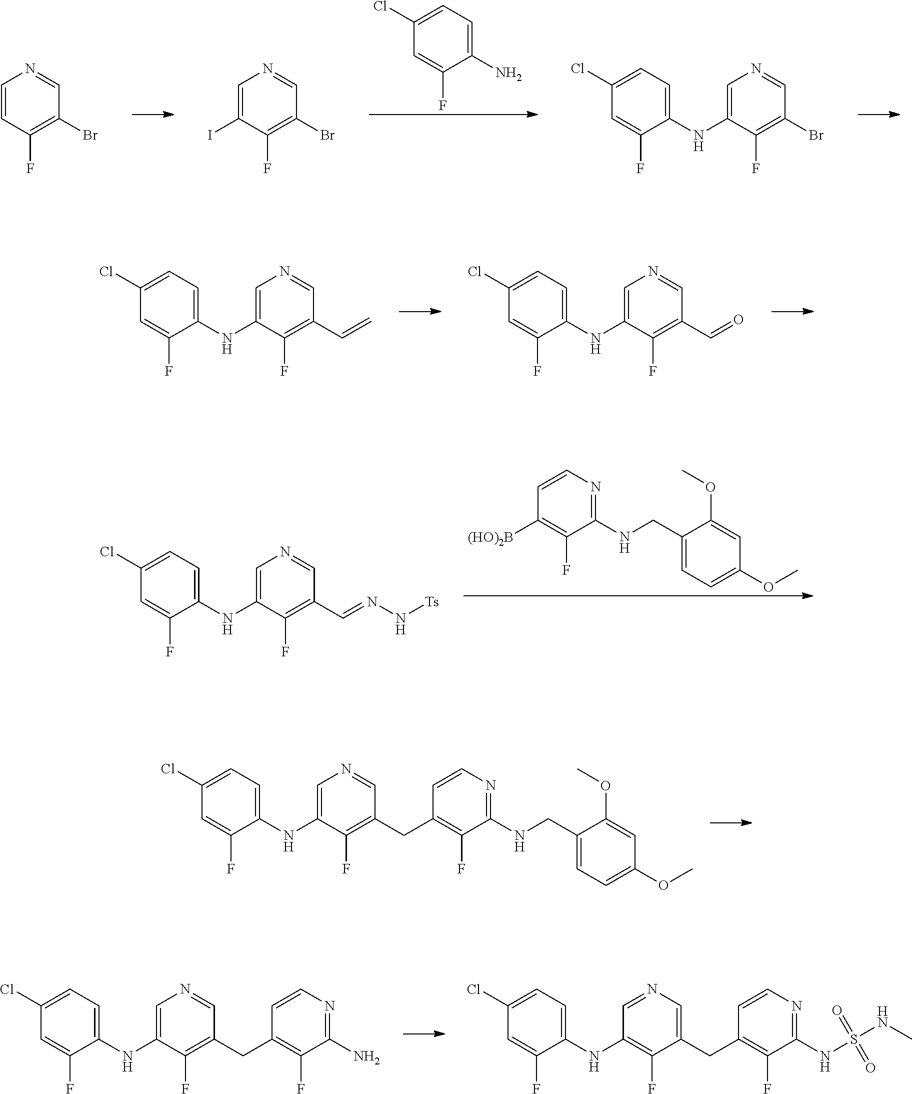

Step 1: To a solution of LDA (2 M in THF, 18.47 mL) in THF (80 mL) was added a solution of 3-bromo-4-fluoro-pyridine (5 g, 28.41 mmol) in THF (20 mL) was added. The mixture was stirred at −75° C. for 3 hr. Then a solution of I₂ (7.21 g, 28.41 mmol, 5.72 mL) in THF (20 mL) was added. The mixture was stirred at −75° C. for 2 hr. Water (50 mL) was added and the mixture were extracted with EtOAc (30 mL×2). The organic layer was dried over anhydrous Na₂SO₄, filtered and concentrated. The residue was purified by flash column chromatography on silica gel (EtOAc in PE=0-6%) to give 3-bromo-4-fluoro-5-iodo-pyridine (5 g, 16.56 mmol). ¹H NMR (400 MHz, DMSO-d₆) δ=8.85 (d, J=7.6 Hz, 1H), 8.73 (d, J=8.8 Hz, 1H). ¹⁹F NMR (376.5 MHz, DMSO-d₆) δ=−80.545.

Step 2: To a solution of 3-bromo-4-fluoro-5-iodo-pyridine (3.6 g, 11.93 mmol) in 1,4-dioxane (70 mL) were added 4-chloro-2-fluoro-aniline (1.74 g, 11.93 mmol), Pd(OAc)₂ (267.73 mg, 1.19 mmol), Xantphos (1.38 g, 2.39 mmol) and Cs₂CO₃ (7.77 g, 23.85 mmol). The mixture was stirred at 80° C. for 4 hr. Water (30 mL) was added and the mixture were extracted with EtOAc (20 mL×2). The organic layer was dried over anhydrous Na₂SO₄, filtered and concentrated. The crude was purified by flash column chromatography on silica gel (EtOAc in PE=0-7%) to give 5-bromo-N-(4-chloro-2-fluoro-phenyl)-4-fluoro-pyridin-3-amine (2.3 g, 7.20 mmol). ¹H NMR (400 MHz, DMSO-d₆) δ=8.42 (s, 1H), 8.40 (d, J=8.4 Hz, 1H), 8.21 (dd, J=1.2, 9.6 Hz, 1H), 7.46 (dd, J=2.4, 11.2 Hz, 1H), 7.21 (d, J=8.8 Hz, 1H), 7.12-7.07 (m, 1H). ¹⁹F NMR (376.5 MHz, DMSO-d₆) δ=−112.378, −122.188.

Step 3: To a solution of 5-bromo-N-(4-chloro-2-fluoro-phenyl)-4-fluoro-pyridin-3-amine (0.78 g, 2.44 mmol) in 1,4-dioxane (10 mL) and H₂O (2 mL) were added 4,4,5,5-tetramethyl-2-vinyl-1,3,2-dioxaborolane (451.15 mg, 2.93 mmol, 496.86 μL), K₂CO₃ (1.01 g, 7.32 mmol) and Pd(dppf)Cl₂ (178.61 mg, 244.11 μmol). The mixture was stirred at 80° C. for 4 hr. Water (40 mL) was added and the mixture were extracted with EtOAc (20 mL×2). The organic layer was dried over anhydrous Na₂SO₄, filtered and concentrated. The residue was purified by flash column chromatography on silica gel (EtOAc in petroleum ether=0-17%) to give N-(4-chloro-2-fluoro-phenyl)-4-fluoro-5-vinyl-pyridin-3-amine (0.58 g, 2.17 mmol). 1H NMR (400 MHz, CDCl₃) δ=8.42 (d, J=6.4 Hz, 1H), 8.18 (d, J=9.2 Hz, 2H), 7.44 (dd, J=2.0, 11.2 Hz, 1H), 7.16 (d, J=8.0 Hz, 1H), 7.02-6.97 (m, 1H), 6.84-6.76 (m, 1H), 6.04 (d, J=18.0 Hz, 1H), 5.56 (d, J=11.6 Hz, 1H). ¹⁹F NMR (376.5 MHz, CDCl₃) δ=−123.398, −123.413.

Step 4: To a solution of N-(4-chloro-2-fluoro-phenyl)-4-fluoro-5-vinyl-pyridin-3-amine (0.58 g, 2.17 mmol) in THF (32 mL) and H₂O (8 mL) were add K₂OsO₄·2H₂O (80.14 mg, 217.49 umol) and NaIO₄ (1.86 g, 8.70 mmol, 482.07 μL). The mixture was stirred at 25° C. for 1 hr. Water (10 mL) was added and the mixture were extracted with EtOAc (10 mL×2). The organic layer was dried over anhydrous Na₂SO₄, filtered and concentrated. The crude was purified by flash column chromatography on silica gel (EtOAc in petroleum ether=0-25%) to give 5-(4-chloro-2-fluoro-anilino)-4-fluoro-pyridine-3-carbaldehyde (0.34 g, 1.27 mmol). 1H NMR (400 MHz, CDCl₃) δ=10.39 (s, 1H), 8.64 (t, J=9.6 Hz, 2H), 7.23-7.13 (m, 3H), 5.78 (s, 1H). ¹⁹F NMR (376.5 MHz, CDCl₃) δ=−125.228, −131.315.

Step 5: To a solution of 5-(4-chloro-2-fluoro-anilino)-4-fluoro-pyridine-3-carbaldehyde (0.3 g, 1.12 mmol) in MeOH (3 mL) was added 4-methylbenzenesulfonohydrazide (207.97 mg, 1.12 mmol). The mixture was stirred at 60° C. for 1 hr. The residue was filtered and the filter cake was washed with MeOH (5 mL). The filter cake was dried to give N-[(E)-[5-(4-chloro-2-fluoro-anilino)-4-fluoro-3-pyridyl]methyleneamino]-4-methyl-benzenesulfonamide (0.34 g, 778.28 μmol). ¹H NMR (400 MHz, CDCl₃) δ=11.79 (s, 1H), 8.38 (d, J=8.4 Hz, 1H), 8.26-8.22 (m, 2H), 8.03 (s, 1H), 7.77 (d, J=8.4 Hz, 2H), 7.45-7.41 (m, 3H), 7.14 (d, J=8.4 Hz, 1H), 7.20 (t, J=8.4 Hz, 1H), 2.37 (s, 3H). ¹⁹F NMR (376.5 MHz, CDCl₃) δ=−123.032, −123.973.

Step 6: To a solution of N-[(E)-[5-(4-chloro-2-fluoro-anilino)-4-fluoro-3-pyridyl]methyleneamino]-4-methyl-benzenesulfonamide (0.26 g, 595.15 μmol) in dioxane (5 mL) were added [2-[(2,4-dimethoxyphenyl)methylamino]-3-fluoro-4-pyridyl]boronic acid (728.70 mg, 1.19 mmol, example 32) and K₂CO₃ (246.76 mg, 1.79 mmol). The mixture was stirred at 110° C. for 2 hr. Water (30 mL) was added and the mixture were extracted with EtOAc (20 ml×2). The organic layer was dried over anhydrous Na₂SO₄, filtered and concentrated. The crude was purified by flash column chromatography on silica gel (EtOAc in petroleum ether=0-17%) to give 4-[[5-(4-chloro-2-fluoro-anilino)-4-fluoro-3-pyridyl]methyl]-N-[(2,4-dimethoxyphenyl)methyl]-3-fluoro-pyridin-2-amine (0.12 g, 233.04 μmol). LCMS Rₜ=0.793 min in 1.5 min chromatography, 5-95AB, ESI calcd. For C₂₆H₂₃ClF₃N₄O₂ [M+H]⁺515.1, found 515.0.

Step 7: To a solution of 4-[[5-(4-chloro-2-fluoro-anilino)-4-fluoro-3-pyridyl]methyl]-N-[(2,4-dimethoxyphenyl)methyl]-3-fluoro-pyridin-2-amine (0.08 g, 155.36 μmol) in DCM (2 mL) was added TFA (513.72 mg, 4.51 mmol, 333.58 μL). The mixture was stirred at 25° C. for 2 hr. Water (20 mL) was added and the mixture were extracted with EtOAc (10 mL×2). The organic layer was dried over anhydrous Na₂SO₄, filtered and concentrated. The crude was purified by flash column chromatography on silica gel (EtOAc in petroleum ether=0-30%) to give 4-[[5-(4-chloro-2-fluoro-anilino)-4-fluoro-3-pyridyl]methyl]-3-fluoro-pyridin-2-amine (0.05 g, 137.08 μmol). ¹H NMR (400 MHz, DMSO-d₆) δ=8.17 (t, J=7.2 Hz, 3H), 7.65 (d, J=5.2 Hz, 1H), 7.44 (dd, J=2.0, 11.2 Hz, 1H), 7.17 (d, J=8.4 Hz, 1H), 6.98 (t, J=8.8 Hz, 1H), 6.37 (d, J=4.8 Hz, 1H), 6.16 (s, 2H), 3.97 (s, 2H). ¹⁹F NMR (376.5 MHz, DMSO-d₆) δ=−121.716, −122.988, −145.462.

Step 8: To a solution of 4-[[5-(4-chloro-2-fluoro-anilino)-4-fluoro-3-pyridyl]methyl]-3-fluoro-pyridin-2-amine (0.03 g, 82.25 μmol) in DMA (1.5 mL) and MeCN (1.5 mL) were added N-methylsulfamoyl chloride (53.28 mg, 411.24 μmol) and Py (65.06 mg, 822.48 μmol, 66.39 uL). The mixture was stirred at 40° C. for 1 hr. The mixture was concentrated under reduced pressure. The crude product was purified by Pre-HPLC (column: Boston Prime C18 150×30 mm×5 um; mobile phase: [water(NH₃H₂O+NH₄HCO₃)−ACN]; B %: 27%-57%, 7 min) to give 4-[[5-(4-chloro-2-fluoro-anilino)-4-fluoro-3-pyridyl]methyl]-3-fluoro-N-(methylsulfamoyl)pyridin-2-amine (9 mg, 19.66 μmol). ¹H NMR (400 MHz, DMSO-d₆) δ=8.17-8.12 (m, 3H), 7.99 (d, J=4.0 Hz, 1H), 7.39 (dd, J=2.0, 11.2 Hz, 1H), 7.16-7.15 (m, 1H), 7.15-7.13 (m, 1H), 6.97-6.91 (m, 1H), 4.05 (s, 2H), 2.33 (s, 3H). ¹⁹F NMR (376.5 MHz, DMSO-d₆) δ=−121.548, −122.913, −138.740. LCMS Rₜ=0.702 min 1.5 min chromatography, 5-95AB, ESI calcd. for C₁₈H₁₆ClF₃N₅O₂S [M+H]⁺458.1 found 458.0.

### Example 27: {[4-({5-[(5-chloropyridin-2-yl)oxy]-4-methylpyridin-3-yl}methyl)-3-fluoropyridin-2-yl]sulfamoyl}(methyl)amine

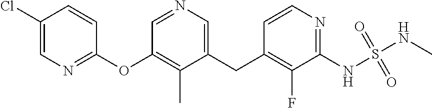 [SMILES: CNS(=O)(=O)Nc1nccc(Cc2cncc(Oc3ccc(Cl)cn3)c2C)c1F]

### Route

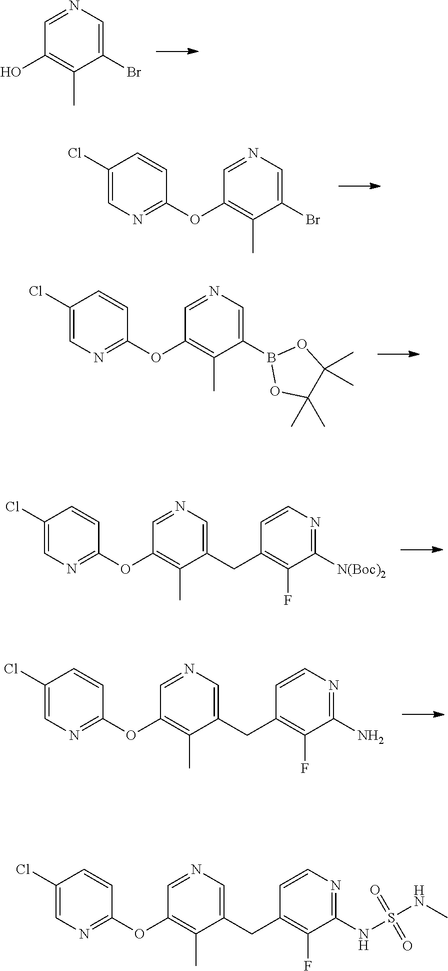

Step 1: To a stirred solution of 5-bromo-4-methylpyridin-3-ol (2 g, 10.637 mmol, example 19) and 5-chloro-2-fluoropyridine (2.80 g, 21.274 mmol) in DMSO (20 mL) was added Cs₂CO₃ (13.86 g, 42.548 mmol, 4 equiv) at room temperature. The resulting mixture was stirred for 16 h at 60° C. Desired product could be detected by LCMS. The reaction mixture was diluted with water (100 mL). The resulting mixture was extracted with EA (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (5:1) to afford 3-bromo-5-[(5-chloropyridin-2-yl)oxy]-4-methylpyridine (1 g). LCMS: (ESI, m/z): [M+1]⁺=298.75. ¹H NMR (400 MHz, Chloroform-d) δ 8.56 (s, 1H), 8.26 (s, 1H), 8.04 (d, J=2.7 Hz, 1H), 7.70 (dd, J=8.7, 2.6 Hz, 1H), 6.98 (d, J=8.7 Hz, 1H), 2.26 (s, 3H).

Step 2: A mixture of 3-bromo-5-[(5-chloropyridin-2-yl)oxy]-4-methylpyridine (500 mg, 1.669 mmol), 4,4,5,5-tetramethyl-2-(4,4,5,5-tetramethyl-1,3,2-dioxaborolan-2-yl)-1,3,2-dioxaborolane (508.64 mg, 2.003 mmol, 1.2 equiv), AcOK (327.63 mg, 3.338 mmol, 2.0 equiv) and Pd(PPh₃)₂Cl₂ (117.16 mg, 0.167 mmol, 0.1 equiv) in dioxane (5 mL) was stirred for 16 h at 80° C. under nitrogen atmosphere. Desired product could be detected by LCMS. The resulting mixture was diluted with water (50 mL). The resulting mixture was extracted with EA (3×50 mL). The combined organic layers were washed with brine (50 mL), dried over anhydrous Na₂SO₄. After filtration, the filtrate was concentrated under reduced pressure. This resulted in 3-[(5-chloropyridin-2-yl)oxy]-4-methyl-5-(4,4,5,5-tetramethyl-1,3,2-dioxaborolan-2-yl)pyridine, LCMS: (ESI, m/z): [M+1]⁺=347.2

Step 3: A mixture of 3-[(5-chloropyridin-2-yl)oxy]-4-methyl-5-(4,4,5,5-tetramethyl-1,3,2-dioxaborolan-2-yl)pyridine (153.95 mg, 0.444 mmol), tert-butyl N-[4-(bromomethyl)-3-fluoropyridin-2-yl]-N-(tert-butoxycarbonyl) carbamate (90 mg, 0.222 mmol, 0.5 equiv), K₂CO₃ (92.08 mg, 0.666 mmol, 1.5 equiv) and Pd(dppf)Cl₂ (16.25 mg, 0.022 mmol, 0.05 equiv) in dioxane (3 mL) and H₂O (0.3 mL) was stirred for 1 h at 80° C. under nitrogen atmosphere. Desired product could be detected by LCMS. The reaction mixture was diluted with water (20 mL). The resulting mixture was extracted with EA (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (5:1) to afford tert-butyl N-(tert-butoxycarbonyl)-N-[4-({5-[(5-chloropyridin-2-yl)oxy]-4-methylpyridin-3-yl}methyl)-3-fluoropyridin-2-yl]carbamate (120 mg). LCMS: (ESI, m/z): [M+1]⁺=545.3. ¹H NMR (400 MHz, Chloroform-d) δ 8.30 (s, 1H), 8.20 (d, J=4.9 Hz, 1H), 8.07-7.99 (m, 2H), 7.69 (dd, J=8.7, 2.6 Hz, 1H), 7.01-6.88 (m, 2H), 4.13 (s, 2H), 2.05 (s, 3H), 1.41 (s, 18H). ¹⁹F NMR (376 MHz, Chloroform-d) δ −130.99.

Step 4: To a stirred solution of tert-butyl N-(tert-butoxycarbonyl)-N-[4-({5-[(5-chloropyridin-2-yl)oxy]-4-methylpyridin-3-yl}methyl)-3-fluoropyridin-2-yl]carbamate (240 mg, 0.440 mmol) in DCM (4 mL) was added TFA (1 mL) at 0° C. The resulting mixture was stirred for 2 h at room temperature. Desired product could be detected by LCMS. The resulting mixture was basified to PH 8 with sat. NaHCO₃ (aq.). The resulting mixture was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 60% gradient in 20 min; detector, UV 254 nm. This resulted in 4-({5-[(5-chloropyridin-2-yl)oxy]-4-methylpyridin-3-yl}methyl)-3-fluoropyridin-2-amine (114 mg). LCMS: (ESI, m/z): [M+1]⁺=345.1. ¹H NMR (400 MHz, Chloroform-d) δ 8.28 (d, J=1.4 Hz, 2H), 8.05 (dd, J=2.6, 0.7 Hz, 1H), 7.73 (d, J=5.3 Hz, 1H), 7.68 (dd, J=8.7, 2.7 Hz, 1H), 6.96 (dd, J=8.7, 0.7 Hz, 1H), 6.30 (t, J=5.1 Hz, 1H), 4.79-4.70 (m, 2H), 4.01 (s, 2H), 2.07 (s, 3H). ¹⁹F NMR (376 MHz, Chloroform-d) −144.99.

Step 5: To a stirred solution of 4-({5-[(5-chloropyridin-2-yl)oxy]-4-methylpyridin-3-yl}methyl)-3-fluoropyridin-2-amine (50 mg, 0.145 mmol) and pyridine (114.71 mg, 1.450 mmol, 10 equiv) in DMA (0.5 mL) was added N-methylsulfamoyl chloride (22.55 mg, 0.174 mmol, 1.2 equiv) in DMA (0.5 mL) dropwise at 0° C. under nitrogen atmosphere. The resulting mixture was stirred for 2 h at room temperature under nitrogen atmosphere. Desired product could be detected by LCMS. The reaction mixture was purified by Prep-HPLC with the following conditions: Column: XBridge Prep Phenyl OBD Column, 19*250 mm, 5 um; Mobile Phase A: Water (10 mmol/L NH₄HCO₃), Mobile Phase B: MeOH-HPLC; Flow rate: 50 mL/min; Gradient: 53% B to 68% B in 8 min; Wave Length: 254/220 nm; RT1 (min): 9.22. This resulted in {[4-({5-[(5-chloropyridin-2-yl)oxy]-4-methylpyridin-3-yl}methyl)-3-fluoropyridin-2-yl]sulfamoyl}(methyl)amine (36 mg). LCMS: (ESI, m/z): [M+1]⁺=438.10. ¹H NMR (400 MHz, Methanol-d₄) δ 8.23 (m, 2H), 8.01 (m, 2H), 7.87 (dd, J=8.7, 2.6 Hz, 1H), 7.12 (d, J=8.7 Hz, 1H), 6.72 (t, J=5.1 Hz, 1H), 4.18 (s, 2H), 2.63 (s, 3H), 2.11 (s, 3H). ¹⁹F NMR (376 MHz, Methanol-d₄) δ −142.128

### Example 28: N-(4-chloro-2-fluorophenyl)-5-[(3-fluoro-2-methanesulfonylpyridin-4-yl)methyl]-4-methylpyridin-3-amine

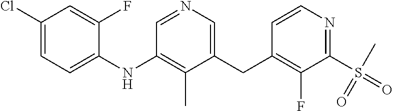 [SMILES: Cc1c(Cc2ccnc(S(C)(=O)=O)c2F)cncc1Nc1ccc(Cl)cc1F]

### Route

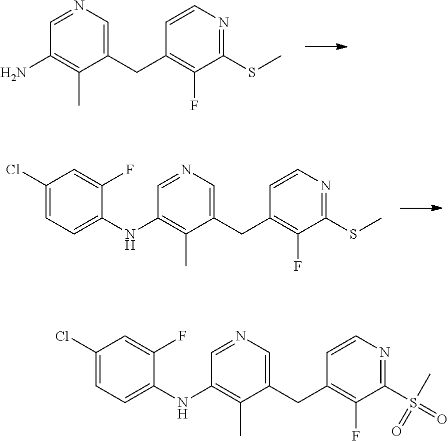 [SMILES: CSc1nccc(Cc2cncc(N)c2C)c1F ; CSc1nccc(Cc2cncc(Nc3ccc(Cl)cc3F)c2C)c1F ; Cc1c(Cc2ccnc(S(C)(=O)=O)c2F)cncc1Nc1ccc(Cl)cc1F]

Step 1: A mixture of 5-{[3-fluoro-2-(methylsulfanyl)pyridin-4-yl]methyl}-4-methylpyridin-3-amine (40 mg, 0.152 mmol, example 9), 4-chloro-2-fluoro-1-iodobenzene (58.43 mg, 0.228 mmol, 1.5 equiv), XantPhos (8.79 mg, 0.015 mmol, 0.1 equiv), Pd₂(dba)₃ (13.91 mg, 0.015 mmol, 0.1 equiv) and Cs₂CO₃ (98.98 mg, 0.304 mmol) in toluene (1 mL) was stirred for 2 h at 100° C. Desired product could be detected by LCMS. The resulting mixture was concentrated under reduced pressure. The residue was purified by silica gel column chromatography, eluted with PE/EA (1:1) to afford N-(4-chloro-2-fluorophenyl)-5-{[3-fluoro-2-(methylsulfanyl)pyridin-4-yl]methyl}-4-methylpyridin-3-amine (10 mg). LCMS: (ESI, m/z): [M+1]⁺=391.9. ¹H NMR (400 MHz, Chloroform-d) δ 8.41 (s, 1H), 8.22-8.12 (m, 2H), 7.17-7.09 (m, 1H), 7.01-6.94 (m, 1H), 6.78-6.69 (m, 1H), 6.64-6.57 (m, 1H), 5.42 (s, 1H), 4.02 (s, 2H), 2.59 (s, 3H), 2.12 (s, 3H). ¹⁹F NMR (377 MHz, Chloroform-d) δ −127.03, −130.44.

Step 2: To a stirred solution of N-(4-chloro-2-fluorophenyl)-5-{[3-fluoro-2-(methylsulfanyl)pyridin-4-yl]methyl}-4-methylpyridin-3-amine (10 mg, 0.026 mmol) in acetone (1 mL), MeOH (1 mL) and H₂O (0.1 mL) was added oxone (17.17 mg, 0.104 mmol, 4 equiv) in portions at 0° C. under air atmosphere. The resulting mixture was stirred for 24 h at room temperature under air atmosphere. Desired product could be detected by LCMS. The reaction was quenched with sat. Na₂S₂O₃ (aq.) at 0° C., the resulting mixture was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 80% gradient in 25 min; detector, UV 254 nm. This resulted in N-(4-chloro-2-fluorophenyl)-5-[(3-fluoro-2-methanesulfonylpyridin-4-yl)methyl]-4-methylpyridin-3-amine (5.9 mg). LCMS: (ESI, m/z): [M+1]⁺=424.20. ¹H NMR (400 MHz, Methanol-d₄) δ 8.40 (d, J=4.8 Hz, 1H), 8.15-8.09 (m, 2H), 7.40 (t, J=5.1 Hz, 1H), 7.24-7.15 (m, 1H), 7.08-7.00 (m, 1H), 6.75 (t, J=8.9 Hz, 1H), 4.29 (s, 2H), 3.37 (s, 3H), 2.16 (s, 3H). ¹⁹F NMR (377 MHz, Methanol-d₄) δ −125.80, −127.80.

### Example 29: {[4-({5-[(5-chloro-3-fluoropyridin-2-yl)oxy]-4-methylpyridin-3-yl}methyl)-3-fluoropyridin-2-yl]sulfamoyl}(methyl)amine

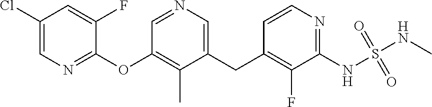 [SMILES: CNS(=O)(=O)Nc1nccc(Cc2cncc(Oc3ncc(Cl)cc3F)c2C)c1F]

### Route

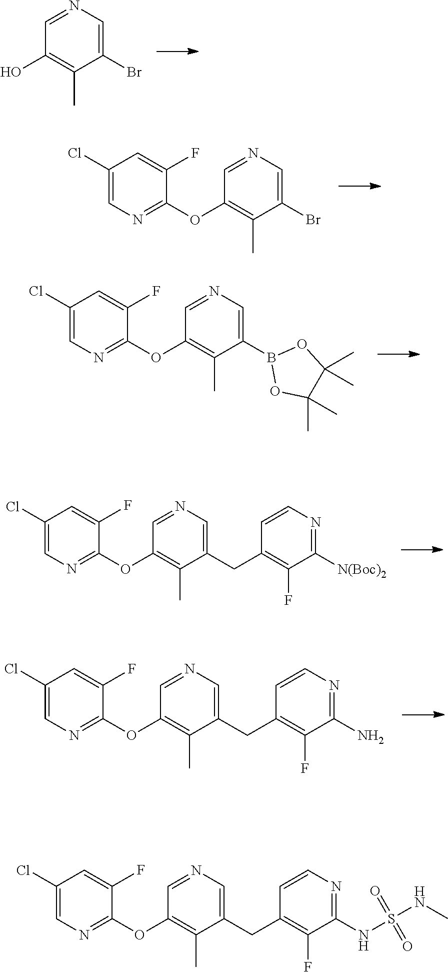

Step 1: To a stirred mixture of 5-bromo-4-methylpyridin-3-ol (1 g, 5.318 mmol, 1 equiv) and Cs₂CO₃ (6.93 g, 21.272 mmol) in DMSO (10 mL) was added 5-chloro-2,3-difluoropyridine (1.59 g, 10.636 mmol) dropwise at room temperature under nitrogen atmosphere. The resulting mixture was stirred for overnight at 80° C. under nitrogen atmosphere. Desired product could be detected by LCMS. The reaction mixture was diluted with water. The resulting mixture was extracted with EA (3×50 mL). The combined organic layers were washed with brine, dried over anhydrous Na₂SO₄. After filtration, the filtrate was concentrated under reduced pressure. The residue was purified by silica gel column chromatography, eluted with PE/EA (6:1) to afford 2-[(5-bromo-4-methylpyridin-3-yl)oxy]-5-chloro-3-fluoropyridine (820 mg). LCMS: (ESI, m/z): [M+1]⁺=317.15. ¹H NMR (400 MHz, Chloroform-d) δ 8.59 (s, 1H), 8.31 (s, 1H), 7.83 (d, J=2.2 Hz, 1H), 7.56 (dd, J=9.0, 2.2 Hz, 1H), 2.29 (s, 3H). ¹⁹F NMR (377 MHz, Chloroform-d) δ −134.14.

Step 2: To a stirred mixture of 2-[(5-bromo-4-methylpyridin-3-yl)oxy]-5-chloro-3-fluoropyridine (200 mg, 0.630 mmol, 1 equiv) and 4,4,5,5-tetramethyl-2-(4,4,5,5-tetramethyl-1,3,2-dioxaborolan-2-yl)-1,3,2-dioxaborolane (191.93 mg, 0.756 mmol) in dioxane (5 mL) were added KOAc (123.63 mg, 1.26 mmol) and Pd(PPh₃)₂Cl₂ (44.21 mg, 0.063 mmol). After stirring for 16 h at 80° C. under a nitrogen atmosphere, the resulting mixture was concentrated under reduced pressure. This resulted in 5-chloro-3-fluoro-2-((4-methyl-5-(4,4,5,5-tetramethyl-1,3,2-dioxaborolan-2-yl)pyridin-3-yl)oxy)pyridine.

Step 3: To a stirred mixture of 3-[(5-chloro-3-fluoropyridin-2-yl)oxy]-4-methyl-5-(4,4,5,5-tetramethyl-1,3,2-dioxaborolan-2-yl)pyridine (200 mg, 0.549 mmol, 1 equiv) and Pd(dppf)Cl₂ (40.14 mg, 0.055 mmol) in dioxane (5 mL) were added tert-butyl N-[4-(bromomethyl)-3-fluoropyridin-2-yl]-N-(tert-butoxycarbonyl) carbamate (111.15 mg, 0.275 mmol, 0.5 equiv) and H₂O (0.1 mL) at room temperature under nitrogen atmosphere. The resulting mixture was stirred for 2 h at room temperature under nitrogen atmosphere. Desired product could be detected by LCMS. The reaction mixture was concentrated under reduced pressure. The residue was purified by silica gel column chromatography, eluted with PE/EA (5:1) to afford tert-butyl N-(tert-butoxycarbonyl)-N-[4-({5-[(5-chloro-3-fluoropyridin-2-yl)oxy]-4-methylpyridin-3-yl}methyl)-3-fluoropyridin-2-yl]carbamate) (150 mg). LCMS: (ESI, m/z): [M+1]⁺=563.3. ¹H NMR (300 MHz, Chloroform-d) δ 8.35 (m, 2H), 8.21 (d, J=4.9 Hz, 1H), 7.85 (dd, J=12.2, 2.2 Hz, 1H), 7.61-7.53 (m, 1H), 6.93 (t, J=5.0 Hz, 1H), 4.12 (d, J=7.6 Hz, 2H), 2.11 (s, 3H), 1.42 (s, 18H). ¹⁹F NMR (282 MHz, Chloroform-d) δ −130.93, −134.13.

Step 4: To a stirred solution of tert-butyl N-(tert-butoxycarbonyl)-N-[4-({5-[(5-chloro-3-fluoropyridin-2-yl)oxy]-4-methylpyridin-3-yl}methyl)-3-fluoropyridin-2-yl]carbamate (215 mg, 0.382 mmol) in DCM (4 mL) were added TFA (1 mL) dropwise at 0° C. under nitrogen atmosphere. The resulting mixture was stirred for 4 h at room temperature under nitrogen atmosphere. Desired product could be detected by LCMS. The reaction mixture was concentrated under reduced pressure. The residue was a basified to pH 10 with sat. NaHCO₃ (aq.). The resulting mixture was extracted with EA (3×30 mL). The combined organic layers were washed with brine (30 mL), dried over anhydrous Na₂SO₄. After filtration, the filtrate was concentrated under reduced pressure. The residue was purified by silica gel column chromatography, eluted with DCM/MeOH (10:1) to afford 4-({5-[(5-chloro-3-fluoropyridin-2-yl)oxy]-4-methylpyridin-3-yl}methyl)-3-fluoropyridin-2-amine (65 mg). LCMS: (ESI, m/z): [M+1]⁺=362.9. ¹H NMR (400 MHz, Chloroform-d) δ 8.41 (s, 1H), 8.32 (s, 1H), 7.83 (d, J=2.2 Hz, 1H), 7.62-7.51 (m, 2H), 6.45 (t, J=6.0 Hz, 1H), 4.14 (d, J=1.4 Hz, 2H), 2.12 (s, 3H). ¹⁹F NMR (377 MHz, Chloroform-d)) δ −134.01, −139.89.

Step 5: To a stirred solution of 4-({5-[(5-chloro-3-fluoropyridin-2-yl)oxy]-4-methylpyridin-3-yl}methyl)-3-fluoropyridin-2-amine (30 mg, 0.083 mmol) and pyridine (65.42 mg, 0.830 mmol) in DMA (0.2 mL) were added N-methylsulfamoyl chloride (21.3 mg, 0.16 mmol) in DMA (0.2 mL) dropwise at 0° C. under nitrogen atmosphere. The resulting mixture was stirred for 1 h at room temperature under nitrogen atmosphere. Desired product could be detected by LCMS. The reaction mixture was purified by reversed-phase flash chromatography with the following conditions: column, C18 silica gel; mobile phase, ACN in Water (10 mmol/L NH₄HCO₃), 5% to 60% gradient in 40 min; detector, UV 254 nm. This resulted in {[4-({5-[(5-chloro-3-fluoropyridin-2-yl)oxy]-4-methylpyridin-3-yl}methyl)-3-fluoropyridin-2-yl]sulfamoyl}(methyl)amine (12.4 mg) LCMS: (ESI, m/z): [M+1]⁺=456.05. ¹H NMR (400 MHz, Methanol-d₄) δ 8.26 (s, 2H), 7.88 (d, J=7.0 Hz, 3H), 6.55 (t, J=5.2 Hz, 1H), 4.14 (s, 2H), 2.59 (s, 3H), 2.13 (s, 3H). ¹⁹F NMR (377 MHz, Methanol-d₄) δ −136.569, −141.502.

### Example 30: 4-[(5-allyloxy-4-methyl-3-pyridyl)methyl]-3-fluoro-N-(methylsulfamoyl)pyridin-2-amine

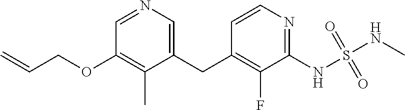 [SMILES: C=CCOc1cncc(Cc2ccnc(NS(=O)(=O)NC)c2F)c1C]

### Route

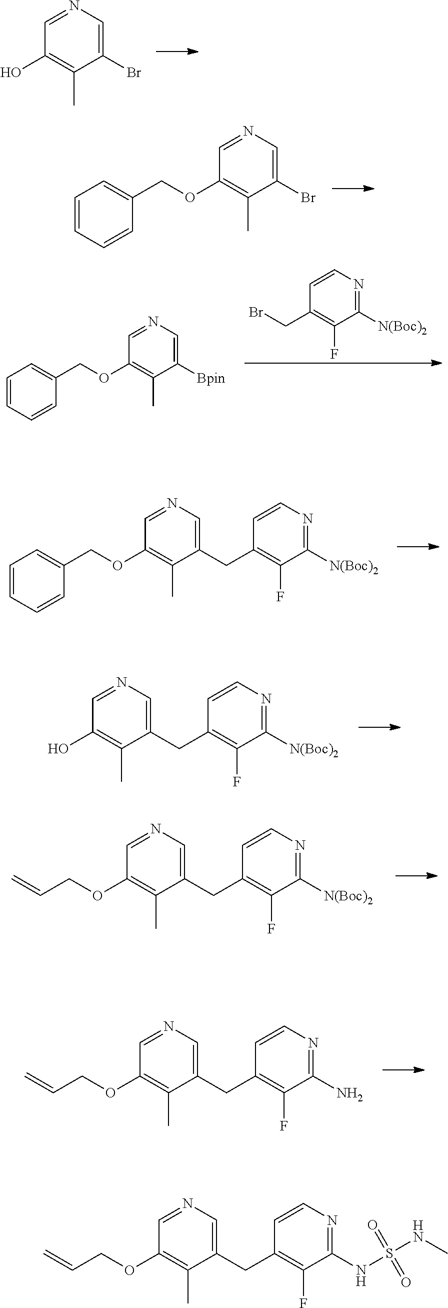

Step 1: To a solution of 5-bromo-4-methyl-pyridin-3-ol (10 g, 53.19 mmol) in DMF (110 mL) was added Cs₂CO₃ (34.66 g, 106.37 mmol) and BnBr (8.19 g, 47.87 mmol, 5.69 mL). The mixture was stirred at 90° C. for 1 h. The reaction was poured into water (500 mL), extracted with EtOAc (300 mL×2). The combined organic layers were washed with brine (500 mL×2), dried over anhydrous Na₂SO₄, filtered and concentrated. The residue was purified by flash chromatography on silica gel (Ethyl acetate in Petroleum ether=0-10%) to give 3-benzyloxy-5-bromo-4-methyl-pyridine (4 g, 14.38 mmol). ¹H NMR (400 MHz, CDCl₃) δ=8.35 (s, 1H), 8.16 (s, 1H), 7.43-7.34 (m, 5H), 5.17 (s, 2H), 2.39 (s, 3H).

Step 2: A mixture of 3-benzyloxy-5-bromo-4-methyl-pyridine (4 g, 14.38 mmol), Pd(dppf)Cl₂ (1.05 g, 1.44 mmol) 4,4,5,5-tetramethyl-2-(4,4,5,5-tetramethyl-1,3,2-dioxaborolan-2-yl)-1,3,2-dioxaborolane (5.48 g, 21.57 mmol) and KOAc (4.23 g, 43.14 mmol) in dioxane (45 mL) was stirred at 110° C. stirred for 4 h. After cooling to room temperature, the mixture was filtered and the filter cake was washed with EtOAc (30 mL×2). The filtrate was concentrated. The residue was purified by flash chromatography on silica gel (ethyl acetate in petroleum ether=0-80%) to give 3-benzyloxy-4-methyl-5-(4,4,5,5-tetramethyl-1,3,2-dioxaborolan-2-yl)pyridine (1.3 g, 4.00 mmol). ¹H NMR (400 MHz, DMSO-d₆) δ=8.36 (s, 1H), 8.31 (s, 1H), 7.48-7.32 (m, 5H), 5.24 (s, 2H), 2.38 (s, 3H), 1.31 (s, 12H).

Step 3: To a solution of 3-benzyloxy-4-methyl-5-(4,4,5,5-tetramethyl-1,3,2-dioxaborolan-2-yl)pyridine (1.3 g, 4.00 mmol), tert-butyl N-[4-(bromomethyl)-3-fluoro-2-pyridyl]-N-tert-butoxycarbonyl-carbamate (2.11 g, 5.20 mmol), Cs₂CO₃ (2.60 g, 7.99 mmol) in a mixed solvent of toluene (20 mL) and H₂O (4 mL) was added Pd(dppf)Cl₂·CH₂Cl₂ (326.45 mg, 399.74 μmol) under N₂. The mixture was stirred at 100° C. for 2 h. After cooling to room temperature, the mixture was filtered and the filter cake was washed with EtOAc (20 mL×2), the filtrate was concentrated. The residue was purified by flash chromatography on silica gel (Ethyl acetate in Petroleum ether=0-60%) to give tert-butyl N-[4-[(5-benzyloxy-4-methyl-3-pyridyl)methyl]-3-fluoro-2-pyridyl]-N-tert-butoxycarbonyl-carbamate (800 mg, 1.53 mmol). ¹H NMR (400 MHz, CDCl₃) δ=8.24 (s, 1H), 8.18 (d, J=5.2 Hz, 1H), 8.10 (s, 1H), 7.42-7.35 (m, 5H), 6.86 (t, J=5.2 Hz, 1H), 5.18 (s, 2H), 4.09 (s, 2H), 2.18 (s, 3H), 1.41 (s, 18H). ¹⁹F NMR (376.5 MHz, CDCl₃) δ=−130.974.

Step 4: To a solution of tert-butyl N-[4-[(5-benzyloxy-4-methyl-3-pyridyl)methyl]-3-fluoro-2-pyridyl]-N-tert-butoxycarbonyl-carbamate (800 mg, 1.53 mmol) in MeOH (15 mL) was added wet. Pd/C (800 mg, 751.74 μmol, 10% purity) under N₂. The suspension was degassed under vacuum and purged with H₂ several times. The mixture was stirred under H₂ (50 psi) at 50° C. for 16 h. The reaction mixture was filtered and the filter cake was washed with MeOH (20 mL×3), the filtrate was concentrated. The residue was purified by flash chromatography on silica gel (Ethyl acetate in Petroleum ether=0-60%) to give tert-butyl N-tert-butoxycarbonyl-N-[3-fluoro-4-[(5-hydroxy-4-methyl-3-pyridyl)methyl]-2-pyridyl]carbamate (300 mg, 692.09 μmol). ¹H NMR (400 MHz, CDCl₃) δ=8.41-8.31 (m, 1H), 8.23-8.16 (m, 1H), 7.88 (s, 1H), 6.90 (t, J=4.8 Hz, 1H), 4.09 (s, 2H), 3.49 (s, 1H), 2.21 (s, 3H), 1.42 (s, 18H). ¹⁹F NMR (376.5 MHz, CDCl₃) δ=−130.881.

Step 5: To a solution of tert-butyl N-[4-[(5-benzyloxy-4-methyl-3-pyridyl)methyl]-3-fluoro-2-pyridyl]-N-tert-butoxycarbonyl-carbamate (800 mg, 1.53 mmol) in MeOH (15 mL) was added wet. Pd/C (800 mg, 751.74 μmol, 10% purity) under N₂. The suspension was degassed under vacuum and purged with H₂ several times. The mixture was stirred under H₂ (50 psi) at 50° C. for 16 h. The reaction mixture was filtered and the filter cake was washed with MeOH (20 mL×3), the filtrate was concentrated. The residue was purified by flash chromatography on silica gel (Ethyl acetate in Petroleum ether=0-60%) to give tert-butyl N-tert-butoxycarbonyl-N-[3-fluoro-4-[(5-hydroxy-4-methyl-3-pyridyl)methyl]-2-pyridyl]carbamate (300 mg, 692.09 μmol). ¹H NMR (400 MHz, CDCl₃) δ=8.41-8.31 (m, 1H), 8.23-8.16 (m, 1H), 7.88 (s, 1H), 6.90 (t, J=4.8 Hz, 1H), 4.09 (s, 2H), 3.49 (s, 1H), 2.21 (s, 3H), 1.42 (s, 18H). ¹⁹F NMR (376.5 MHz, CDCl₃) δ=−130.881.

Step 6: A solution of tert-butyl N-[4-[(5-allyloxy-4-methyl-3-pyridyl)methyl]-3-fluoro-2-pyridyl]-N-tert-butoxycarbonyl-carbamate (150 mg, 316.77 μmol) in HCl/MeOH (4 M, 2 mL) was stirred at 25° C. for 5 h. The mixture was concentrated. to give 4-[(5-allyloxy-4-methyl-3-pyridyl)methyl]-3-fluoro-pyridin-2-amine (86.57 mg, 316.75 μmol). ¹H NMR (400 MHz, DMSO-d₆) δ=8.53 (d, J=17.2 Hz, 2H), 8.47-8.05 (m, 2H), 7.77 (d, J=6.4 Hz, 1H), 6.61 (t, J=6.4 Hz, 1H), 6.18-5.97 (m, 1H), 5.46 (dd, J=1.2, 17.2 Hz, 1H), 5.34 (dd, J=1.2, 10.4 Hz, 1H), 4.84 (d, J=5.2 Hz, 2H), 4.32 (s, 2H), 2.31 (s, 3H). ¹⁹F NMR (376.5 MHz, DMSO-d₆) δ=−137.046.

Step 7: To a solution of 4-[(5-allyloxy-4-methyl-3-pyridyl)methyl]-3-fluoro-pyridin-2-amine (75 mg, 274.42 μmol) and Py (217.07 mg, 2.74 mmol, 221.49 μL) in MeCN (5 mL) was added N-methylsulfamoyl chloride (71.11 mg, 548.84 μmol) under N₂. The mixture was stirred at 25° C. stirred for 2 h. The mixture was concentrated. The residue was purified by flash chromatography on silica gel (MeOH in DCM=0-10%) and then purified by SFC (column: DAICEL CHIRALCEL OJ (250 mm*30 mm, 10 μm); mobile phase: [CO₂-EtOH (0.1% NH₃H₂O)]; B %: 20%, isocratic elution mode) to give 4-[(5-allyloxy-4-methyl-3-pyridyl)methyl]-3-fluoro-N-(methylsulfamoyl)pyridin-2-amine (35.1 mg, 95.79 μmol). 1H NMR (400 MHz, CDCl₃) δ=8.39-8.06 (m, 2H), 7.96 (br s, 1H), 6.60 (br s, 1H), 6.27-5.91 (m, 1H), 5.55-5.42 (m, 2H), 5.37 (d, J=10.4 Hz, 1H), 4.68 (s, 2H), 4.06 (s, 2H), 2.77 (s, 3H), 2.26 (s, 3H). ¹⁹F NMR (376.5 MHz, CDCl₃) δ=−142.189. LCMS Rₜ=1.348 min in 3 min chromatography, 0-60CD, ESI calcd. for C₁₆H₂₀FN₄O₃S [M+H]⁺367.1, found 367.2.

### Example 31: 3-fluoro-4-[[5-(3-fluoro-2-pyridyl)-4-methyl-3-pyridyl]methyl]-N-(methylsulfamoyl)pyridin-2-amine

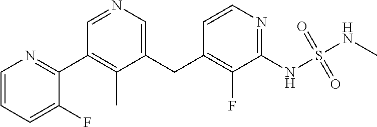 [SMILES: CNS(=O)(=O)Nc1nccc(Cc2cncc(-c3ncccc3F)c2C)c1F]

### Route

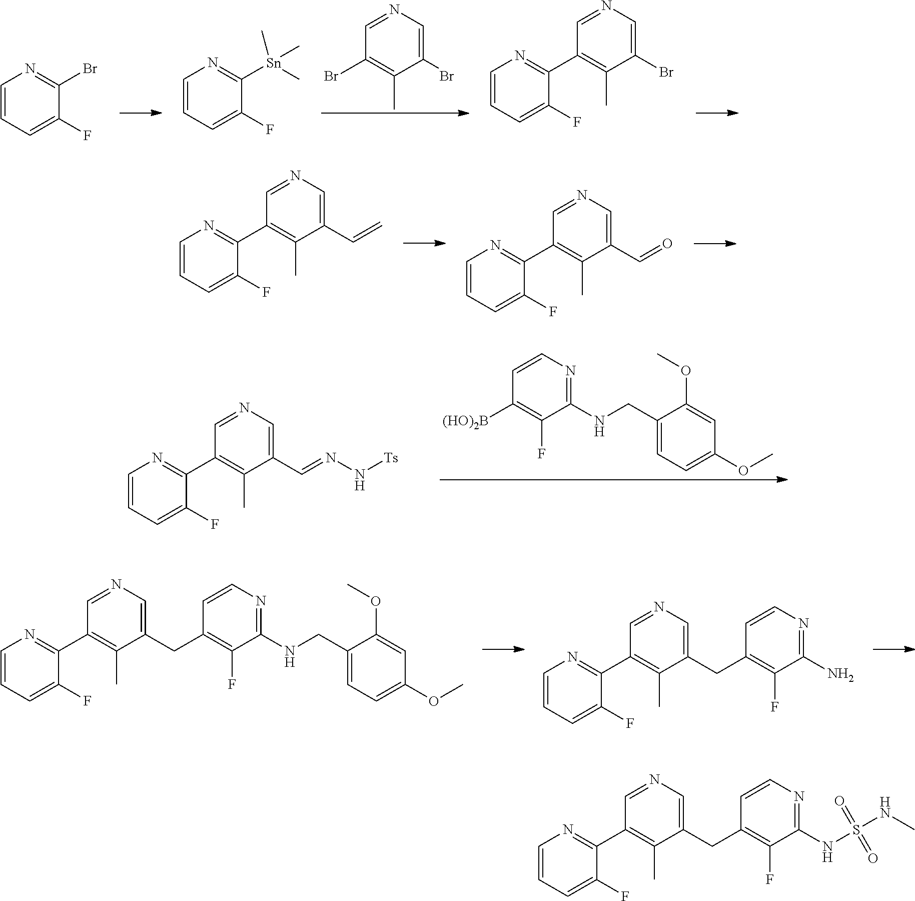

Step 1 and 2: To a solution of 2-bromo-3-fluoro-pyridine (5 g, 28.41 mmol) in dioxane (50 mL) was added Pd(PPh₃)₄ (3.28 g, 2.84 mmol) and trimethyl(trimethylstannyl) stannane (19.19 g, 58.57 mmol, 12.15 mL). The mixture was stirred at 80° C. for 4 h. The mixture was cooled to room temperature. Then the Pd(PPh₃)₄ (3.11 g, 2.69 mmol) and CuI (2.05 g, 10.77 mmol), LiCl (2.28 g, 53.87 mmol, 1.10 mL) was added the mixture. The mixture was stirred at 80° C. for 12 h. Water (100 mL) was added to the mixture and the mixture was extracted with EtOAc (150 mL×2). The combined organic phase was washed with brine (100 mL), dried over anhydrous Na₂SO₄, filtered and concentrated. The crude product was purified by flash chromatography on silica gel (EtOAc in DCM=0-15%) and (EtOAc in PE=0-20%) to give 3-bromo-5-(3-fluoro-2-pyridyl)-4-methyl-pyridine (500 mg, 1.87 mmol). ¹H NMR (400 MHz, DMSO-d₆) δ=8.80 (s, 1H), 8.60 (d, J=4.4 Hz, 1H), 8.49 (s, 1H), 7.98-7.89 (m, 1H), 7.66-7.59 (m, 1H), 2.25 (s, 3H). ¹⁹F NMR (376.5 MHz, DMSO-d₆) δ=−121.916

Step 3: To a solution of 3-bromo-5-(3-fluoro-2-pyridyl)-4-methyl-pyridine (500 mg, 1.87 mmol) in dioxane (6 mL) and H₂O (1.2 mL) was added 4,4,5,5-tetramethyl-2-vinyl-1,3,2-dioxaborolane (634.28 mg, 4.12 mmol, 698.55 mL), K₂CO₃ (776.16 mg, 5.62 mmol) and Pd(dppf)Cl₂ (273.95 mg, 374.40 mmol). The mixture was stirred at 90° C. for 12 h. Water (20 mL) was added. The mixture were extracted with EtOAc (30 mL×3). The combined organic layers were washed with brine (30 mL), dried over anhydrous Na₂SO₄, filtered and concentrated. The crude product was purified by flash chromatography on silica gel (EtOAc in PE=0-50%) to give 3-(3-fluoro-2-pyridyl)-4-methyl-5-vinyl-pyridine (390 mg, 1.82 mmol). ¹H NMR (400 MHz, CDCl₃) δ=8.67 (s, 1H), 8.58-8.50 (m, 1H), 8.47 (s, 1H), 7.58-7.50 (m, 1H), 7.42-7.35 (m, 1H), 6.92 (dd, J=10.8, 17.6 Hz, 1H), 5.74 (d, J=17.6 Hz, 1H), 5.47 (d, J=11.2 Hz, 1H), 2.23 (s, 3H). ¹⁹F NMR (376.5 MHz, CDCl₃) δ=−120.931

Step 4: To a solution of 3-(3-fluoro-2-pyridyl)-4-methyl-5-vinyl-pyridine (390 mg, 1.82 mmol) in THF (16 mL) and H₂O (4 mL) was added dipotassium; dioxido (dioxo) osmium; dihydrate (67.07 mg, 182.04 mmol) and NaIO₄ (1.95 g, 9.10 mmol, 504.36 mL). The mixture was stirred at 25° C. for 0.5 h. Water (20 mL) was added. The mixture were extracted with EtOAc (30 mL×3). The combined organic layers were washed with brine (30 mL), dried over anhydrous Na₂SO₄, filtered and concentrated. The crude product was purified by flash chromatography on silica gel (EtOAc in PE=0-55%) to give 5-(3-fluoro-2-pyridyl)-4-methyl-pyridine-3-carbaldehyde (240 mg, 1.11 mmol). 1H NMR (400 MHz, CDCl₃) δ=10.40 (s, 1H), 9.02 (s, 1H), 8.76 (s, 1H), 8.61-8.58 (m, 1H), 7.66-7.55 (m, 1H), 7.48-7.42 (m, 1H), 2.59 (s, 3H). ¹⁹F NMR (376.5 MHz, CDCl₃) δ=−120.720

Step 5: To a solution of 5-(3-fluoro-2-pyridyl)-4-methyl-pyridine-3-carbaldehyde (310 mg, 1.43 mmol) in MeOH (4 mL) was added 4-methylbenzenesulfonohydrazide (267.02 mg, 1.43 mmol). The mixture was stirred at 60° C. for 1 h. The mixture was concentrated. N-[(E)-[5-(3-fluoro-2-pyridyl)-4-methyl-3-pyridyl]methyleneamino]-4-methyl-benzenesulfonamide (551.19 mg, 1.43 mmol). ¹H NMR (400 MHz, CDCl₃) δ=8.76 (s, 1H), 8.59-8.56 (m, 1H), 8.49 (s, 1H), 8.04 (s, 1H), 7.86 (d, J=8.4 Hz, 2H), 7.58-7.52 (m, 1H), 7.45-7.40 (m, 1H), 7.32 (d, J=8.0 Hz, 2H), 2.41 (s, 3H), 2.27 (s, 3H). ¹⁹F NMR (376.5 MHz, CDCl₃) δ=−120.699

Step 6: To a solution of N-[(E)-[5-(3-fluoro-2-pyridyl)-4-methyl-3-pyridyl]methyleneamino]-4-methyl-benzenesulfonamide (551.19 mg, 1.43 mmol) in dioxane (6 mL) was added [2-[(2,4-dimethoxyphenyl)methylamino]-3-fluoro-4-pyridyl]boronic acid (877.76 mg, 2.87 mmol, example 30) and K₂CO₃ (594.48 mg, 4.30 mmol). The mixture was stirred at 100° C. for 2 h. Then the mixture was stirred at 110° C. for 2 h. Water (20 mL) was added. The mixture were extracted with EtOAc (30 mL×3). The combined organic layers were washed with brine (30 mL), dried over anhydrous Na₂SO₄, filtered and concentrated. The crude product was purified by flash chromatography on silica gel (EtOAc in PE=0-57%) to give N-[(2,4-dimethoxyphenyl)methyl]-3-fluoro-4-[[5-(3-fluoro-2-pyridyl)-4-methyl-3-pyridyl]methyl]pyridin-2-amine (200 mg, 432.44 mmol). LCMS Rₜ=4.098 min 8 min chromatography, 10-80CD, ESI calcd. for C₂₆H₂₅F₂N₄O₂ [M+H]⁺463.2 found 463.2.

Step 7: To a solution of N-[(2,4-dimethoxyphenyl)methyl]-3-fluoro-4-[[5-(3-fluoro-2-pyridyl)-4-methyl-3-pyridyl]methyl]pyridin-2-amine (200 mg, 432.44 mmol) in DCM (1 mL) was added TFA (1.54 g, 13.51 mmol, 1 mL). The mixture was stirred at 25° C. for 2 h. Anhydrous NaHCO₃ (20 mL) was added. The mixture were extracted with EtOAc (30 mL×3). The combined organic layers were washed with brine (30 mL), dried over anhydrous Na₂SO₄, filtered and concentrated. The crude product was purified by flash chromatography on silica gel (EtOAc in PE=0-100%) and Prep-TLC (EA=100%) to

### Example 32: 4-[[5-(5-chloro-3-fluoro-2-pyridyl)-4-methyl-3-pyridyl]methyl]-3-fluoro-N-(methylsulfamoyl)pyridin-2-amine

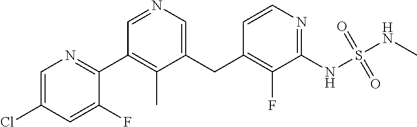 [SMILES: CNS(=O)(=O)Nc1nccc(Cc2cncc(-c3ncc(Cl)cc3F)c2C)c1F]

### Route

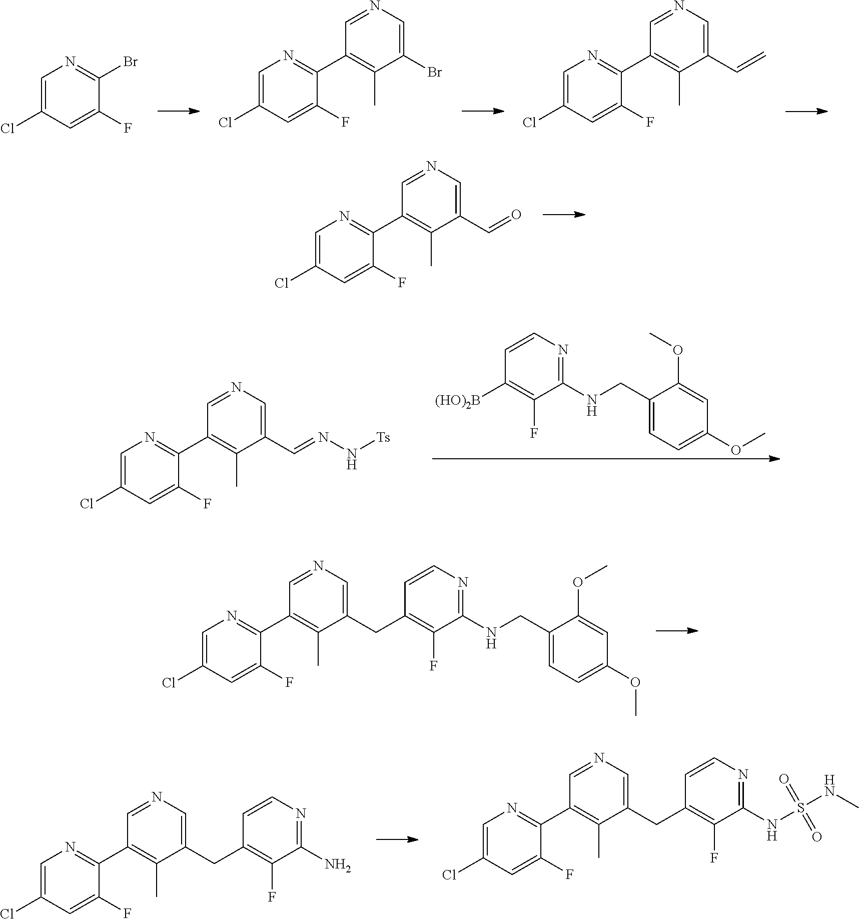

Step 1a and 1b: A solution of 2-bromo-5-chloro-3-fluoro-pyridine (1 g, 4.75 mmol) in dioxane (10 mL) was added trimethyl(trimethylstannyl) stannane (4.67 g, 14.26 mmol, 2.96 mL) and Pd(PPh₃)₄ (823.71 mg, 712.82 mmol). The mixture was stirred at 90° C. for 4 hr. The mixture was concentrated to give (5-chloro-3-fluoro-2-pyridyl)-trimethyl-stannane (1.39 g, 4.72 mmol). 3,5-dibromo-4-methyl-pyridine (1.18 g, 4.72 mmol) in dioxane (10 mL) were added above mixture, then (5-chloro-3-fluoro-2-pyridyl)-trimethyl-stannane (1.39 g, 4.72 mmol), CuI (359.75 mg, 1.89 mmol), LiCl (400.37 mg, 9.44 mmol, 193.41 mL) and Pd(PPh₃)₄ (545.70 mg, 472.24 mmol) was added the mixture under N₂. The mixture was stirred at 80° C. for 2 h. Water (50 mL) was added and the mixture were extracted with EtOAc (50 mL×2). The organic layer was dried over anhydrous Na₂SO₄, filtered and concentrated. The crude was purified by flash column chromatography on silica gel (EtOAc in petroleum ether=0-10%) to give 2-(5-bromo-4-methyl-3-pyridyl)-5-chloro-3-fluoro-pyridine (570 mg, 1.89 mmol). ¹H NMR (400 MHz, CDCl₃) δ=8.75 (s, 1H), 8.56-8.54 (m, 1H), 8.46 (s, 1H), 7.62 (dd, J=8.0, 4.0 Hz, 1H), 2.33 (d, J=1.6 Hz, 3H).

Step 2: To a solution of 2-(5-bromo-4-methyl-3-pyridyl)-5-chloro-3-fluoro-pyridine (400 mg, 1.33 mmol) in dioxane (6 mL) and H₂O (1.2 mL) were added 4,4,5,5-tetramethyl-2-vinyl-1,3,2-dioxaborolane (449.46 mg, 2.92 mmol, 495.00 mL), K₂CO₃ (550.00 mg, 3.98 mmol) and Pd(dppf)Cl₂ (194.12 mg, 265.30 mmol). The mixture was stirred at 80° C. for 2 h. Water (20 mL) was added and the mixture were extracted with EtOAc (20 mL×2). The organic layer was dried over anhydrous Na₂SO₄, filtered and concentrated. The crude was purified by flash column chromatography on silica gel (EtOAc in petroleum ether=0-30%) to give 3-(5-chloro-3-fluoro-2-pyridyl)-4-methyl-5-vinyl-pyridine (290 mg, 1.17 mmol). 1H NMR (400 MHz, CDCl₃) δ=8.69 (s, 1H), 8.55 (s, 1H), 8.46 (s, 1H), 7.63-7.58 (m, 1H), 6.91 (dd, J=11.2, 17.6, Hz, 1H), 5.76 (d, J=17.2 Hz, 1H), 5.51 (d, J=11.2 Hz, 1H), 2.25 (s, 3H).

Step 3: To a solution of 3-(5-chloro-3-fluoro-2-pyridyl)-4-methyl-5-vinyl-pyridine (290 mg, 1.17 mmol) in THF (4 mL) and H₂O (0.8 mL) were added K₂OsO₄·2H₂O (42.97 mg, 116.61 mmol) and NaIO₄ (997.71 mg, 4.66 mmol, 258.47 mL). The mixture was stirred at 25° C. for 1 hr. The resulting solution was diluted water (20 mL) and quenched with saturated Na₂SO₃ solution until KI test paper turn to white. The mixture was filtered. Water (20 mL) was added and the mixture were extracted with EtOAc (20 mL×2). The organic layer was dried over anhydrous Na₂SO₄, filtered and concentrated. The crude was purified by flash column chromatography on silica gel (EtOAc in petroleum ether=0-30%) to give 5-(5-chloro-3-fluoro-2-pyridyl)-4-methyl-pyridine-3-carbaldehyde (110 mg, 438.85 mmol). ¹H NMR (400 MHz, CDCl₃) δ=10.39 (s, 1H), 9.03 (s, 1H), 8.73 (s, 1H), 8.58 (d, J=1.2 Hz, 1H), 7.64 (dd, J=2.0, 8.8 Hz, 1H), 2.59-2.56 (m, 3H).

Step 4: To a solution of 5-(5-chloro-3-fluoro-2-pyridyl)-4-methyl-pyridine-3-carbaldehyde (110 mg, 438.85 mmol) in MeOH (2 mL) were added 4-methylbenzenesulfonohydrazide (81.73 mg, 438.85 mmol). The mixture was stirred at 60° C. for 1 h. Water (20 mL) was added and the mixture were extracted with EtOAc (20 mL×2). The organic layer was dried over anhydrous Na₂SO₄, filtered and concentrated to give N-[(E)-[5-(5-chloro-3-fluoro-2-pyridyl)-4-methyl-3-pyridyl]methyleneamino]-4-methyl-benzenesulfonamide (183 mg, 436.89 mmol). ¹H NMR (400 MHz, CDCl₃) δ=8.85 (s, 1H), 8.58-8.51 (m, 3H), 8.11 (s, 1H), 7.90 (d, J=8.4 Hz, 2H), 7.63 (dd, J=2.4, 8.8 Hz, 1H), 7.35 (d, J=8.0 Hz, 2H), 2.44 (s, 3H), 2.32 (s, 3H).

Step 5: To a solution of N-[(E)-[5-(5-chloro-3-fluoro-2-pyridyl)-4-methyl-3-pyridyl]methyleneamino]-4-methyl-benzenesulfonamide (183 mg, 436.89 mmol) in dioxane (2 mL) were added [2-[(2,4-dimethoxyphenyl)methylamino]-3-fluoro-4-pyridyl]boronic acid (534.92 mg, 873.78 mmol, 50% purity) and K₂CO₃ (181.14 mg, 1.31 mmol). The mixture was stirred at 110° C. for 2 h. Water (20 mL) was added and the mixture was extracted with EtOAc (20 mL×2). The organic layer was dried over anhydrous Na₂SO₄, filtered and concentrated. The crude was purified by flash column chromatography on silica gel (EtOAc in petroleum ether=0-40%) to give 4-[[5-(5-chloro-3-fluoro-2-pyridyl)-4-methyl-3-pyridyl]methyl]-N-[(2,4-dimethoxyphenyl)methyl]-3-fluoro-pyridin-2-amine (340 mg, 232.63 mmol, 53.25% yield). LCMS Rt=0.747 min in 1.5 min chromatography, 5-95AB, ESI calcd. for C₂₆H₂₄ClN₄F₂O₂ [M+H]⁺497.1, found 497.0.

Step 6: To a solution of 4-[[5-(5-chloro-3-fluoro-2-pyridyl)-4-methyl-3-pyridyl]methyl]-N-[(2,4-dimethoxyphenyl)methyl]-3-fluoro-pyridin-2-amine (340 mg, 232.63 mmol, 34% purity) in DCM (2 mL) were added TFA (2.26 g, 19.84 mmol, 1.47 mL). The mixture was stirred at 25° C. for 2 h. The mixture was adjusted to pH>7 by NH₃-MeOH (7 M, 10 mL) and the mixture was concentrated. Then water (20 mL) was added and the mixture were extracted with DCM (20 mL×2). The organic layer was dried over anhydrous Na₂SO₄, filtered and concentrated. The crude was purified by flash column chromatography on silica gel (EtOAc in petroleum ether=0-100%) and triturated with DCM (20 mL) to give 4-[[5-(5-chloro-3-fluoro-2-pyridyl)-4-methyl-3-pyridyl]methyl]-3-fluoro-pyridin-2-amine (30 mg, 86.51 mmol). LCMS Rt=1.453 min in 1.5 min chromatography, 5-95AB, ESI calcd. for C₁₇H₁₄ClN₄F₂ [M+H]⁺347.1, found 347.1.

Step 7: To a solution of 4-[[5-(5-chloro-3-fluoro-2-pyridyl)-4-methyl-3-pyridyl]methyl]-3-fluoro-pyridin-2-amine (12 mg, 34.61 mmol) in DMA (1 mL) and MeCN (1 mL) were added N-methylsulfamoyl chloride (44.84 mg, 346.06 mmol) and Py (27.37 mg, 346.06 mmol, 27.93 mL). The mixture was stirred at 20° C. for 2 hr. The mixture was concentrated. The crude was purified by prep-HPLC (column: Phenomenex C18 80×40 mm×3 mm; mobile phase: [water(NH₃H₂O+NH₄HCO₃)−ACN]; B %: 20%-50%, 7 min) to give 4-[[5-(5-chloro-3-fluoro-2-pyridyl)-4-methyl-3-pyridyl]methyl]-3-fluoro-N-(methylsulfamoyl)pyridin-2-amine (2.1 mg, 4.77 μmol). ¹H NMR (400 MHz, CD₃CN) δ=8.78-8.19 (m, 3H), 7.94-7.92 (s, 1H), 7.84 (dd, J=9.2, 2.0 Hz, 1H), 6.77-6.64 (m, 1H), 4.14 (s, 2H), 2.62-2.50 (m, 3H), 2.07 (s, 3H). ¹⁹F NMR (376.5 MHz, CD₃CN) δ=−120.53, −141.74 ppm. LCMS Rₜ=0.712 min in 1.5 min chromatography, 5-95AB, ESI calcd. for C₁₈H₁₇ClN₅F₂O₂S [M+H]⁺440.1, found 439.9.

### Example 33: 3-fluoro-4-({5-[(2-fluoro-4-methylphenyl)amino]-4-methylpyridin-3-yl}methyl)pyridin-2-amine

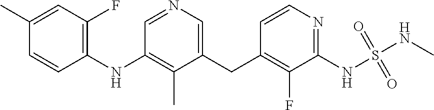

### Route

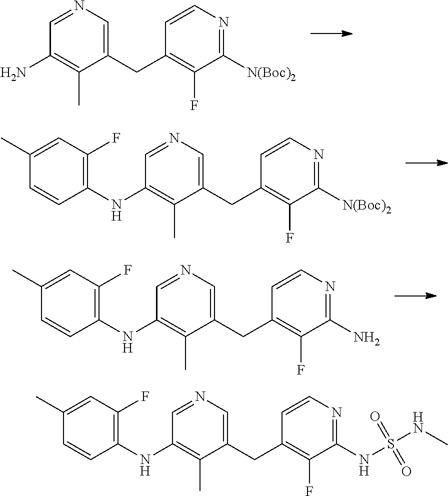

Step 1: To a stirred mixture of tert-butyl N-{4-[(5-amino-4-methylpyridin-3-yl)methyl]-3-fluoropyridin-2-yl}-N-(tert-butoxycarbonyl) carbamate (180 mg, 0.416 mmol, example 6) and 1-bromo-2-fluoro-4-methylbenzene (118.01 mg, 0.624 mmol, 1.5 equiv) in dioxane (3 mL) were added Cs₂CO₃ (271.20 mg, 0.832 mmol), Pd₂(dba)₃ (38.11 mg, 0.042 mmol, 0.1 equiv) and XantPhos (24.08 mg, 0.042 mmol, 0.1 equiv). After stirring for 4 h at 80° C. under a nitrogen atmosphere, the reaction mixture was diluted with water (15 mL). The resulting mixture was extracted with EA (3×15 mL). The combined organic layers were washed with brine (15 mL), dried over anhydrous Na₂SO₄. After filtration, the filtrate was concentrated under reduced pressure. The residue was purified by silica gel column chromatography, eluted with PE/EA (3:1) to afford tert-butyl N-(tert-butoxycarbonyl)-N-[3-fluoro-4-({5-[(2-fluoro-4-methylphenyl)amino]-4-methylpyridin-3-yl}methyl)pyridin-2-yl]carbamate (130 mg). LCMS: (ESI, m/z): [M+1]⁺=541.40. ¹H NMR (400 MHz, Chloroform-d) δ 8.34 (s, 1H), 8.19 (d, J=5.0 Hz, 1H), 8.09 (s, 1H), 7.00-6.84 (m, 4H), 4.10 (s, 2H), 2.31 (s, 3H), 2.13 (s, 3H), 1.43 (s, 18H).

Step 2: To a stirred solution of tert-butyl N-(tert-butoxycarbonyl)-N-[3-fluoro-4-({5-[(2-fluoro-4-methylphenyl)amino]-4-methylpyridin-3-yl}methyl)pyridin-2-yl]carbamate (109 mg, 0.202 mmol) in DCM (2 mL) was added TFA (0.5 mL) dropwise at 0° C. The resulting mixture was stirred for 2 h at room temperature under nitrogen atmosphere. The reaction mixture was basified to pH 10 with sat. NaHCO₃ (aq.). The resulting mixture was extracted with EA (3×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃) 5% to 70% gradient in 25 min; detector, UV 254 nm. This resulted in 3-fluoro-4-({5-[(2-fluoro-4-methylphenyl)amino]-4-methylpyridin-3-yl}methyl)pyridin-2-amine (60 mg). LCMS: (ESI, m/z): [M+1]⁺=341.05. ¹H NMR (400 MHz, Chloroform-d) δ 8.35 (s, 1H), 8.12 (s, 1H), 7.73 (d, J=5.2 Hz, 1H), 6.93 (d, J=12.0 Hz, 1H), 6.86-6.79 (m, 2H), 6.26 (t, J=5.1 Hz, 1H), 5.32 (s, 1H), 4.62 (s, 2H), 3.99 (s, 2H), 2.30 (s, 3H), 2.14 (s, 3H). ¹⁹F NMR (377 MHz, Chloroform-d) δ −132.30, −145.41.

### Example 34: {[3-fluoro-4-({5-[(3-fluoropyridin-2-yl)methoxy]-4-methylpyridin-3-yl}methyl)pyridin-2-yl]sulfamoyl}(methyl)amine

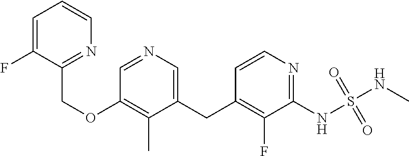 [SMILES: CNS(=O)(=O)Nc1nccc(Cc2cncc(OCc3ncccc3F)c2C)c1F]

### Route

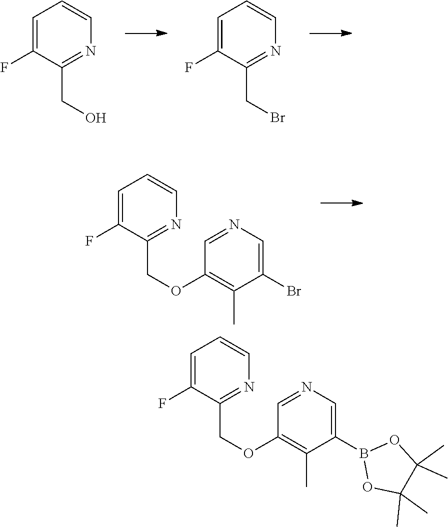

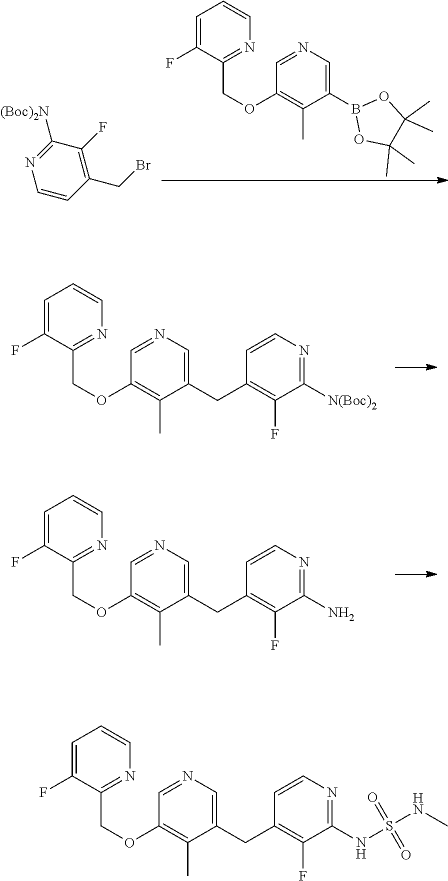

Step 1: To a stirred mixture of (3-fluoropyridin-2-yl) methanol (4 g, 31.467 mmol) and PPh₃ (9.90 g, 37.760 mmol, 1.2 equiv) in DCM (30 mL) were added CBr₄ (12.52 g, 37.760 mmol, 1.2 equiv) in DCM (10 mL) dropwise at 0° C. under nitrogen atmosphere. The resulting mixture was stirred for 4 h at room temperature under nitrogen atmosphere. Desired product could be detected by LCMS. The resulting mixture was concentrated under vacuum. The residue was purified by silica gel column chromatography, eluted with PE/EA (5:1) to afford 2-(bromomethyl)-3-fluoropyridine (3.9 g). LCMS: (ESI, m/z): [M+1]⁺=190.02. ¹H NMR (400 MHz, Chloroform-d) δ 8.40 (m, 1H), 7.41 (m, 1H), 7.31-7.26 (m, 1H), 4.61 (d, J=2.1 Hz, 2H). ¹⁹F NMR (377 MHz, Chloroform-d) δ −122.31.

Step 2: A mixture of 2-(bromomethyl)-3-fluoropyridine (3.9 g, 20.525 mmol), 5-bromo-4-methylpyridin-3-ol (2 g, 10.637 mmol, 0.5 equiv) and K₂CO₃ (7.35 g, 53.185 mmol, 2.5 equiv) in DMF (20 mL) was stirred for 15 min at 80° C. under nitrogen atmosphere. Desired product could be detected by LCMS. The resulting mixture was diluted with water (100 mL). The resulting mixture was extracted with EA (3×100 mL). The combined organic layers were washed with brine (100 mL), dried over anhydrous Na₂SO₄. After filtration, the filtrate was concentrated under reduced pressure. The residue was purified by silica gel column chromatography, eluted with PE/EA (4:1) to afford 3-bromo-5-[(3-fluoropyridin-2-yl)methoxy]-4-methylpyridine (1.2 g). LCMS: (ESI, m/z): [M+1]⁺=297.05. ¹H NMR (400 MHz, Chloroform-d) δ 8.46 (m, 1H), 8.38-8.18 (m, 2H), 7.59-7.41 (m, 1H), 7.36 (m, 1H), 5.35 (m, 2H), 2.44-2.26 (m, 3H). ¹⁹F NMR (377 MHz, Chloroform-d) δ −124.35.

Step 3: To a solution of 3-bromo-5-[(3-fluoropyridin-2-yl)methoxy]-4-methylpyridine (600 mg, 2.019 mmol) and bis(pinacolato)diboron (615.35 mg, 2.423 mmol, 1.2 equiv) in dioxane (3 mL) were added KOAc (396.36 mg, 4.038 mmol) and Pd(PPh₃)₂Cl₂ (141.74 mg, 0.202 mmol, 0.1 equiv). After stirring for 16 h at 80° C. under a nitrogen atmosphere, the resulting mixture was concentrated under reduced pressure. This resulted in 3-((3-fluoropyridin-2-yl) methoxy)-4-methyl-5-(4,4,5,5-tetramethyl-1,3,2-dioxaborolan-2-yl)pyridine as a crude product, which was used in the next step directly without further purification. LCMS: (ESI, m/z): [M+1]⁺=345.2

Step 4: To a stirred mixture of tert-butyl N-[4-(bromomethyl)-3-fluoropyridin-2-yl]-N-(tert-butoxycarbonyl) carbamate (300 mg, 0.740 mmol, 1.00 equiv) and 3-[(3-fluoropyridin-2-yl)methoxy]-4-methyl-5-(4,4,5,5-tetramethyl-1,3,2-dioxaborolan-2-yl)pyridine (509.58 mg, 1.480 mmol) in dioxane (10 mL) and H₂O (1 mL) were added K₂CO₃ (306.92 mg, 2.220 mmol, 3 equiv) and Pd(dppf)Cl₂ (54.17 mg, 0.074 mmol, 0.1 equiv). After stirring for 1 h at 80° C. under a nitrogen atmosphere, the resulting mixture was concentrated under reduced pressure. The residue was purified by reversed-phase flash chromatography with the following conditions: column, C18 silica gel; mobile phase, ACN in Water (10 mmol/L NH₄HCO₃), 5% to 60% gradient in 20 min; detector, UV 254 nm. This resulted in tert-butyl N-(tert-butoxycarbonyl)-N-[3-fluoro-4-({5-[(3-fluoropyridin-2-yl)methoxy]-4-methylpyridin-3-yl}methyl)pyridin-2-yl]carbamate (180 mg). LCMS: (ESI, m/z): [M+1]⁺=543.3

Step 5: To a stirred solution of tert-butyl N-(tert-butoxycarbonyl)-N-[3-fluoro-4-({5-[(3-fluoropyridin-2-yl)methoxy]-4-methylpyridin-3-yl}methyl)pyridin-2-yl]carbamate (180 mg, 0.332 mmol) in DCM (4 mL) were added TFA (1 mL) dropwise at 0° C. The resulting mixture was stirred for 2 h at room temperature. Desired product could be detected by LCMS. The reaction mixture was concentrated under reduced pressure. The residue was basified to pH 10 with sat. NaHCO₃ (aq.). The resulting mixture was extracted with EtOAc (3×20 mL). The combined organic layers were washed with brine (20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 60% gradient in 20 min; detector, UV 254 nm. This resulted in 3-fluoro-4-({5-[(3-fluoropyridin-2-yl)methoxy]-4-methylpyridin-3-yl}methyl)pyridin-2-amine (45 mg). LCMS: (ESI, m/z): [M+1]⁺=342.3. ¹H NMR (400 MHz, Chloroform-d) δ 8.46 (m, 1H), 8.31 (s, 1H), 8.09 (s, 1H), 7.69 (d, J=5.2 Hz, 1H), 7.47 (m, 1H), 7.35 (m, 1H), 6.22 (t, J=5.1 Hz, 1H), 5.34 (d, J=1.9 Hz, 2H), 4.67 (s, 2H), 3.95 (s, 2H), 2.12 (s, 3H). ¹⁹F NMR (376 MHz, Chloroform-d) δ −124.42, −145.28.

Step 6: To a stirred solution of 3-fluoro-4-({5-[(3-fluoropyridin-2-yl)methoxy]-4-methylpyridin-3-yl}methyl)pyridin-2-amine (30 mg, 0.088 mmol) and Pyridine (69.31 mg, 0.880 mmol, 10 equiv) in DMA (0.5 mL) were added N-methylsulfamoyl chloride (13.62 mg, 0.106 mmol, 1.2 equiv) in DMA (0.5 mL) dropwise at 0° C. under nitrogen atmosphere. The resulting mixture was stirred for 2 h at room temperature under nitrogen atmosphere. Desired product could be detected by LCMS. The reaction mixture was purified by Prep-HPLC with the following conditions (Column: XBridge Prep Phenyl OBD Column, 19*250 mm, 5 um; Mobile Phase A: Water (10 mmol/L NH₄HCO₃), Mobile Phase B: ACN; Flow rate: 50 mL/min; Gradient: 8% B to 25% B in 8 min, 25% B; Wave Length: 254/220 nm; RT1 (min): 10.13; Number Of Runs: 0. This resulted in {[3-fluoro-4-({5-[(3-fluoropyridin-2-yl)methoxy]-4-methylpyridin-3-yl}methyl)pyridin-2-yl]sulfamoyl}(methyl)amine (21 mg). LCMS: (ESI, m/z): [M+1]⁺=436.10. ¹H NMR (400 MHz, Methanol-d₄) δ 8.41 (m, 1H), 8.29 (s, 1H), 8.03 (s, 1H), 7.93 (d, J=5.1 Hz, 1H), 7.68 (m, 1H), 7.49 (m, 1H), 6.63 (t, J=5.1 Hz, 1H), 5.37 (d, J=1.9 Hz, 2H), 4.09 (s, 2H), 2.61 (s, 3H), 2.13 (s, 3H). ¹⁹F NMR (376 MHz, Methanol-d₄) δ −126.17, −142.34.

### Example 35: 4-[[5-(4-chloro-2-methoxy-anilino)-4-methyl-3-pyridyl]methyl]-3-fluoro-N-(methylsulfamoyl)pyridin-2-amine

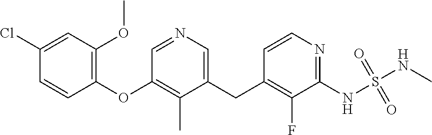 [SMILES: CNS(=O)(=O)Nc1nccc(Cc2cncc(Oc3ccc(Cl)cc3OC)c2C)c1F]

### Route

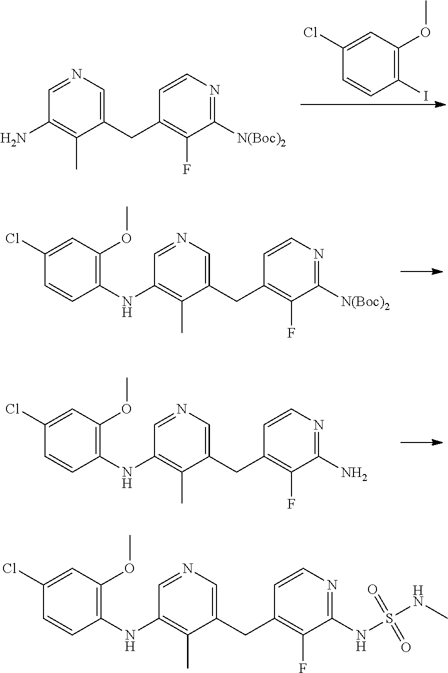

Step 1:4-chloro-1-iodo-2-methoxy-benzene (167.61 mg, 624.30 μmol, example 6) and Pd₂(dba)₃ (19.06 mg, 20.81 μmol) are added under nitrogen to a solution of tert-butyl N-[4-[(5-amino-4-methyl-3-pyridyl)methyl]-3-fluoro-2-pyridyl]-N-tert-butoxycarbonyl-carbamate (180 mg, 416.20 μmol) in dioxane (4.5 mL). The medium is degassed for 5 minutes under N₂ before adding Xantphos (24.08 mg, 41.62 μmol) and Cs₂CO₃ (189.85 mg, 582.68 μmol). The reaction medium was stirred at 100° C. for 8 hours. The mixture was concentrated. The mixture was purified by flash chromatography on silica gel (EtOAc in petroleum ether=0-70%) to afford tert-butyl N-tert-butoxycarbonyl-N-[4-[[5-(4-chloro-2-methoxy-anilino)-4-methyl-3-pyridyl]methyl]-3-fluoro-2-pyridyl]carbamate (200 mg, 349.01 μmol). ¹H NMR (400 MHz, CDCl₃) δ=8.41 (s, 1H), 8.20 (d, J=4.8 Hz, 1H), 8.11 (s, 1H), 6.98-6.67 (m, 4H), 5.74 (s, 1H), 4.10 (s, 2H), 3.90 (s, 3H), 2.13 (s, 3H), 1.43 (s, 18H).

Step 2: A mixture of tert-butyl N-tert-butoxycarbonyl-N-[4-[[5-(4-chloro-2-methoxy-anilino)-4-methyl-3-pyridyl]methyl]-3-fluoro-2-pyridyl]carbamate (200 mg, 349.01 μmol) in HCl/MeOH (4 mL, 4 M) was stirred at 25° C. for 4 h. The mixture was concentrated to give 4-[[5-(4-chloro-2-methoxy-anilino)-4-methyl-3-pyridyl]methyl]-3-fluoro-pyridin-2-amine (142.8 mg, 348.90 μmol).

Step 3: To a solution of 4-[[5-(4-chloro-2-methoxy-anilino)-4-methyl-3-pyridyl]methyl]-3-fluoro-pyridin-2-amine (50 mg, 122.16 μmol, HCl) in MeCN (1 mL) were added Py (96.63 mg, 1.22 mmol, 98.60 μL) and methylsulfamoyl chloride (158.28 mg, 1.22 mmol). The mixture was stirred at 25° C. for 1 h. The reaction mixture quenched with ice water (5 mL). The mixture was extracted with DCM (10 mL×2), combined organic layers were washed with brine (10 mL×2), dried over anhydrous Na₂SO₄ concentrated under reduced pressure. The crude was purified by perp-HPLC (column: Welch Xtimate C18 150×30 mm×5 μm; mobile phase: [water(NH₃H₂O+NH₄HCO₃)−ACN]; B %: 20%-50%, 7 min) to give 4-[[5-(4-chloro-2-methoxy-anilino)-4-methyl-3-pyridyl]methyl]-3-fluoro-N-(methylsulfamoyl)pyridin-2-amine (18.2 mg, 39.06 μmol). ¹H NMR (400 MHz, DMSO-d₆) δ=8.07 (s, 2H), 7.84 (d, J=5.2 Hz, 1H), 7.14-6.96 (m, 2H), 6.80 (d, J=6.4 Hz, 1H), 6.54-6.38 (m, 2H), 4.01 (s, 2H), 3.84 (s, 3H), 2.43 (s, 3H), 2.02 (s, 3H). ¹⁹F NMR (376.5 MHz, DMSO-d₆) δ=−138.699 ppm. LCMS Rₜ=0.753 min in 1.5 min chromatography, 5-95AB, ESI calcd. for C₂₀H₂₂ClFN₅O₃S [M+H]⁺468.1, found 467.8.

### Example 36: N-(4-chloro-2-fluorophenyl)-5-({3-fluoro-2-[(methylsulfamoyl)amino]pyridin-4-yl}methyl)-N, 4-dimethylpyridin-3-amine

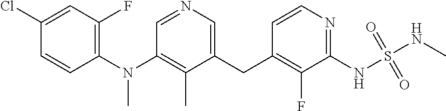 [SMILES: CNS(=O)(=O)Nc1nccc(Cc2cncc(N(C)c3ccc(Cl)cc3F)c2C)c1F]

### Route

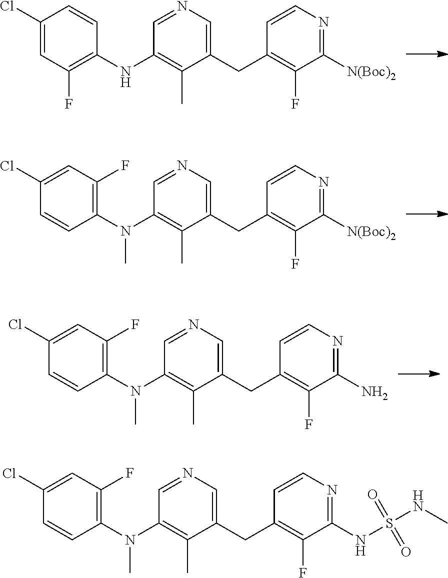

Step 1: A solution of tert-butyl N-(tert-butoxycarbonyl)-N-[4-({5-[(4-chloro-2-fluorophenyl)amino]-4-methylpyridin-3-yl}methyl)-3-fluoropyridin-2-yl]carbamate (88 mg, 0.157 mmol) in DMF (1 mL) was treated with NaH (60 wt %, 12.55 mg, 0.314 mmol) for 10 min at 0° C. under nitrogen atmosphere followed by the addition of Mel (22.26 mg, 0.157 mmol) in DMF (1 mL) dropwise at 0° C. The resulting mixture was stirred for 1 h at room temperature under nitrogen atmosphere. Desired product could be detected by LCMS. The reaction was quenched with sat. NH₄Cl (aq.) at 0° C. The resulting mixture was extracted with EA (3×10 mL). The combined organic layers were washed with brine (1×10 mL), dried over anhydrous Na₂SO₄. After filtration, the filtrate was concentrated under reduced pressure. This resulted in tert-butyl N-(tert-butoxycarbonyl)-N-[4-({5-[(4-chloro-2-fluorophenyl)(methyl)amino]-4-methylpyridin-3-yl}methyl)-3-fluoropyridin-2-yl]carbamate (136 mg). The crude product was used in the next step directly without further purification. LCMS: [M+1]⁺=575.1

Step 2: To a stirred solution of tert-butyl N-(tert-butoxycarbonyl)-N-[4-({5-[(4-chloro-2-fluorophenyl)(methyl)amino]-4-methylpyridin-3-yl}methyl)-3-fluoropyridin-2-yl]carbamate (113 mg, 0.197 mmol) in DCM (4 mL) were added TFA (1 mL) dropwise at 0° C. The resulting mixture was stirred for 2 h at room temperature. Desired product could be detected by LCMS. The reaction mixture was concentrated under reduced pressure. The mixture was basified to pH 10 with sat. NaHCO₃ (aq.). The resulting mixture was extracted with EA (3×20 mL). The combined organic layers were washed with water (5×20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 60% gradient in 20 min; detector, UV 254 nm. This resulted in 4-({5-[(4-chloro-2-fluorophenyl)(methyl)amino]-4-methylpyridin-3-yl}methyl)-3-fluoropyridin-2-amine (14 mg). LCMS: (ESI, m/z): [M+1]⁺=375.2. ¹H NMR (400 MHz, Chloroform-d) δ 8.24 (m, 2H), 7.72 (d, J=5.2 Hz, 1H), 7.10-6.90 (m, 2H), 6.90-6.59 (m, 1H), 6.22 (t, J=5.1 Hz, 1H), 4.84-4.52 (m, 2H), 3.97 (s, 2H), 3.21 (d, J=0.7 Hz, 3H), 2.05 (s, 3H). ¹⁹F NMR (376 MHz, Chloroform-d) δ −120.04, −145.18.

Step 3: To a stirred solution of 4-({5-[(4-chloro-2-fluorophenyl)(methyl)amino]-4-methylpyridin-3-yl}methyl)-3-fluoropyridin-2-amine (10 mg, 0.027 mmol) and pyridine (21.10 mg, 0.270 mmol, 10 equiv) in DMA (0.3 mL) was added N-methylsulfamoyl chloride (4.15 mg, 0.032 mmol, 1.2 equiv) in DMA (0.2 mL) dropwise at 0° C. under nitrogen atmosphere. The resulting mixture was stirred for 2 h at room temperature under nitrogen atmosphere. Desired product could be detected by LCMS. The reaction mixture was purified by Prep-HPLC with following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 26% B to 46% B in 10 min; Wave Length: 254/220 nm; RT1 (min): 11.03. This resulted in N-(4-chloro-2-fluorophenyl)-5-({3-fluoro-2-[(methylsulfamoyl)amino]pyridin-4-yl}methyl)-N, 4-dimethylpyridin-3-amine (6.8 mg). LCMS: (ESI, m/z): [M+1]⁺=468.1. ¹H NMR (400 MHz, Methanol-d₄) δ 8.17 (d, J=6.5 Hz, 2H), 7.96 (d, J=5.2 Hz, 1H), 7.14-7.07 (m, 2H), 7.00 (t, J=8.9 Hz, 1H), 6.64 (s, 1H), 4.14 (s, 2H), 3.22 (s, 3H), 2.63 (s, 3H), 2.12 (s, 3H). ¹⁹F NMR (376 MHz, Methanol-d₄) δ −122.070, −142.358

### Example 37: N-(4-chloro-2-fluorophenyl)-5-({3-fluoro-2-[(methylsulfamoyl)amino]pyridin-4-yl}methyl)-4-methoxypyridin-3-amine

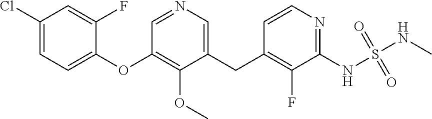 [SMILES: CNS(=O)(=O)Nc1nccc(Cc2cncc(Oc3ccc(Cl)cc3F)c2OC)c1F]

### Route

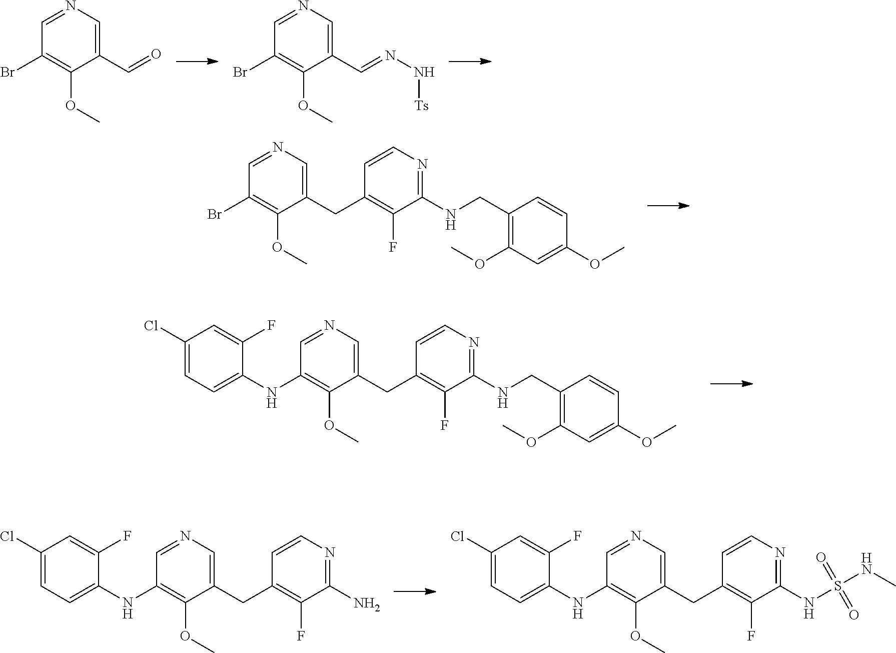 [SMILES: COc1c(Br)cncc1C=O ; COc1c(Br)cncc1/C=N/NS(=O)(=O)c1ccc(C)cc1 ; COc1ccc(CNc2nccc(Cc3cncc(Br)c3OC)c2F)c(OC)c1 ; COc1ccc(CNc2nccc(Cc3cncc(Nc4ccc(Cl)cc4F)c3OC)c2F)c(OC)c1 ; COc1c(Cc2ccnc(N)c2F)cncc1Nc1ccc(Cl)cc1F ; CNS(=O)(=O)Nc1nccc(Cc2cncc(Nc3ccc(Cl)cc3F)c2OC)c1F]

Step 1: A mixture of 5-bromo-4-methoxypyridine-3-carbaldehyde (50 mg, 0.231 mmol) and 4-toluenesulfonyl hydrazide (47.41 mg, 0.254 mmol, 1.1 equiv) in MeOH (1 mL) was stirred for 2 h at room temperature under nitrogen atmosphere. Desired product could be detected by LCMS. The resulting mixture was concentrated under vacuum. The residue was purified by silica gel column chromatography, eluted with DCM/MeOH (10:1) to afford N′-[(1E)-(5-bromo-4-methoxypyridin-3-yl)methylidene]-4-methylbenzenesulfonohydrazide (80 mg). LCMS: (ESI, m/z): [M+1]⁺=384.05. ¹H NMR (400 MHz, Chloroform-d) δ 8.84 (s, 1H), 8.65 (s, 1H), 8.29 (s, 1H), 8.01 (s, 1H), 7.88 (d, J=8.1 Hz, 2H), 7.35 (t, J=8.8 Hz, 2H), 3.95 (s, 3H), 2.43 (s, 3H).

Step 2: A mixture of N′-[(1E)-(5-bromo-4-methoxypyridin-3-yl)methylidene]-4-methylbenzenesulfonohydrazide (70 mg, 0.182 mmol), K₂CO₃ (30.21 mg, 0.218 mmol, 1.2 equiv) and 2-{[(2,4-dimethoxyphenyl)methyl]amino}-3-fluoropyridin-4-ylboronic acid (557.63 mg, 1.820 mmol, 10 equiv) in dioxane (5 mL) was stirred for 1 h at 110° C. under nitrogen atmosphere. Desired product could be detected by LCMS. The resulting mixture was concentrated under vacuum. The residue was purified by reversed-phase flash chromatography with the following conditions: column, C18 silica gel; mobile phase, ACN in Water (0.1% FA), 5% to 70% gradient in 30 min; detector, UV 254 nm. This resulted in 4-[(5-bromo-4-methoxypyridin-3-yl)methyl]-N-[(2,4-dimethoxyphenyl)methyl]-3-fluoropyridin-2-amine (35 mg). LCMS: (ESI, m/z): [M+1]⁺=461.95. ¹H NMR (400 MHz, Chloroform-d) δ 8.59 (s, 1H), 8.31 (s, 1H), 7.80 (d, J=5.6 Hz, 1H), 7.34 (d, J=8.3 Hz, 1H), 6.56-6.38 (m, 3H), 6.34 (s, 1H), 4.71 (s, 2H), 3.94 (s, 2H), 3.88 (s, 3H), 3.85 (s, 3H), 3.80 (s, 3H).

Step 3: To a solution of 4-[(5-bromo-4-methoxypyridin-3-yl)methyl]-N-[(2,4-dimethoxyphenyl)methyl]-3-fluoropyridin-2-amine (204 mg, 0.441 mmol) and 4-chloro-2-fluoroaniline (96.34 mg, 0.661 mmol, 1.5 equiv) in dioxane (10 mL) were added Cs₂CO₃ (287.54 mg, 0.882 mmol), X-Phos (21.04 mg, 0.044 mmol, 0.1 equiv) and Pd₂(dba)₃ (40.41 mg, 0.044 mmol, 0.1 equiv). After stirring for 2 h at 80° C. under a nitrogen atmosphere, the resulting mixture was concentrated under reduced pressure. The residue was purified by silica gel column chromatography, eluted with PE/EA (5:1) to afford N-(4-chloro-2-fluorophenyl)-5-[(2-{[(2,4-dimethoxyphenyl)methyl]amino}-3-fluoropyridin-4-yl)methyl]-4-methoxypyridin-3-amine (182 mg). LCMS: (ESI, m/z): [M+1]⁺=527.50. ¹H NMR (400 MHz, Chloroform-d) δ 8.37 (s, 1H), 8.08 (s, 1H), 7.81 (d, J=5.3 Hz, 1H), 7.28 (m, 1H), 7.20-7.13 (m, 1H), 7.09-7.03 (m, 2H), 6.48 (d, J=2.4 Hz, 1H), 6.43 (dd, J=8.2, 2.4 Hz, 1H), 6.30 (t, J=5.1 Hz, 1H), 5.81 (s, 1H), 5.06 (s, 1H), 4.60 (d, J=5.7 Hz, 2H), 3.94 (s, 2H), 3.85 (s, 3H), 3.80 (s, 3H), 3.77 (s, 3H). ¹⁹F NMR (376 MHz, Chloroform-d) δ −128.20, −147.74.

Step 4: To a stirred solution of N-(4-chloro-2-fluorophenyl)-5-[(2-{[(2,4-dimethoxyphenyl)methyl]amino}-3-fluoropyridin-4-yl)methyl]-4-methoxypyridin-3-amine (172 mg, 0.326 mmol) in DCM (4 mL) were added TFA (1 mL) at 0° C. The resulting mixture was stirred for 1 h at room temperature. Desired product could be detected by LCMS. The reaction mixture was concentrated under reduced pressure. The residue was basified to pH 10 with sat. NaHCO₃ (aq.). The resulting mixture was extracted with EA (3×5 mL). The combined organic layers were washed with water (5 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in 4-({5-[(4-chloro-2-fluorophenyl)amino]-4-methoxypyridin-3-yl}methyl)-3-fluoropyridin-2-amine (60 mg). LCMS: (ESI, m/z): [M+1]⁺=376.90. ¹H NMR (400 MHz, Chloroform-d) δ 8.41 (s, 1H), 8.11 (s, 1H), 7.75 (d, J=5.2 Hz, 1H), 7.18-7.13 (m, 1H), 7.10-7.05 (m, 2H), 6.43 (t, J=5.0 Hz, 1H), 5.79 (s, 1H), 4.64 (s, 2H), 3.98 (s, 2H), 3.76 (d, J=0.9 Hz, 3H). ¹⁹F NMR (376 MHz, Chloroform-d) δ −128.73, −145.54.

Step 5: To a stirred solution of 4-({5-[(4-chloro-2-fluorophenyl)amino]-4-methoxypyridin-3-yl}methyl)-3-fluoropyridin-2-amine (30 mg, 0.080 mmol) and pyridine (62.98 mg, 0.800 mmol, 10 equiv) in DMA (0.8 mL) were added N-methylsulfamoyl chloride (11.35 mg, 0.088 mmol, 1.1 equiv) in DMA (0.2 mL) dropwise at 0° C. under nitrogen atmosphere. The resulting mixture was stirred for 1 h at room temperature under nitrogen atmosphere. Desired product could be detected by LCMS. The crude product was purified by Prep-HPLC with the following conditions: Column: XBridge Shield RP18 OBD Column 30*150 mm, 5 um; Mobile Phase A: Water (10 mmol/L NH₄HCO₃+0.1% NH₃·H₂O), Mobile Phase B: ACN; Flow rate: 60 mL/min mL/min; Gradient: 7% B to 27% B in 8 min; Wavelength: 254 nm/220 nm nm; RT1 (min): 10.08). This resulted in N-(4-chloro-2-fluorophenyl)-5-({3-fluoro-2-[(methylsulfamoyl)amino]pyridin-4-yl}methyl)-4-methoxypyridin-3-amine (16.5 mg). LCMS: (ESI, m/z): [M+1]⁺=470.05. ¹H NMR (400 MHz, Methanol-d₄) δ 8.13-8.07 (m, 2H), 7.98 (s, 1H), 7.21 (m, 1H), 7.05 (m, 1H), 6.85-6.79 (m, 2H), 4.08 (s, 2H), 3.70 (s, 3H), 2.63 (s, 3H). ¹⁹F NMR (377 MHz, Methanol-d₄) δ −127.75, −142.86.

### Example 38: N-(4-chloro-2-fluorophenyl)-5-({3-fluoro-2-[(methylsulfamoyl)amino]pyridin-4-yl}methyl)-4-(trifluoromethyl)pyridin-3-amine

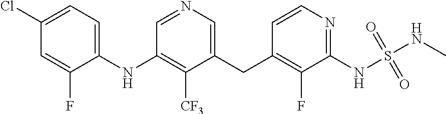 [SMILES: CNS(=O)(=O)Nc1nccc(Cc2cncc(Nc3ccc(Cl)cc3F)c2C(F)(F)F)c1F]

### Route

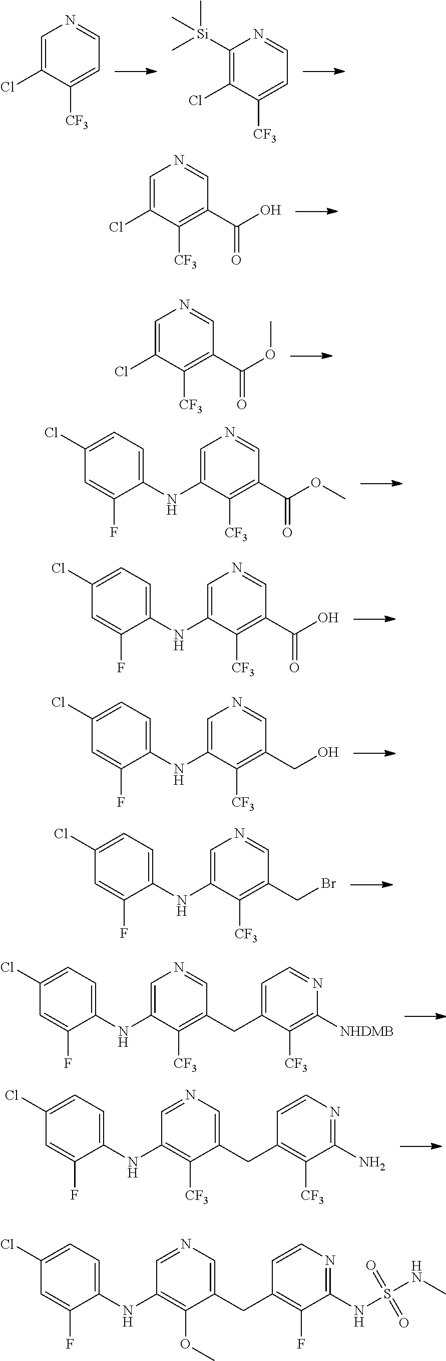

Step 1: To a stirred solution of diisopropylamine (2.79 g, 27.542 mmol) in THF (50 mL) was added n-BuLi in hexanes (11.02 mL, 27.542 mmol) dropwise at −78° C. under nitrogen atmosphere. After keep stirring for 1 h at −78° C., 3-chloro-4-(trifluoromethyl)pyridine (5 g, 27.542 mmol) and TMSCl (2.99 g, 27.542 mmol) in THF (10 mL) were added into the reaction mixture. The resulting mixture was keep stirred for 1 h at −78° C. Desired product could be detected by LCMS. The resulting mixture was quenched by addition of sat. NH₄Cl (aq.) (30 mL) at 0° C. The resulting mixture was extracted with EA (3×50 mL). The combined organic layers were washed with brine (1×50 mL), dried over anhydrous Na₂SO₄. After filtration, the filtrate was concentrated under reduced pressure. The residue in EA was filtered through basic alumina, eluted with diethyl ether (2×30 mL) to afford 3-chloro-4-(trifluoromethyl)-2-(trimethylsilyl)pyridine (5.4 g). LCMS: (ESI, m/z): [M+1]⁺=254.00. ¹H NMR (400 MHz, Chloroform-d) δ 8.83-8.76 (m, 1H), 7.47 (d, J=4.9 Hz, 1H), 0.44 (s, 9H).

Step 2: To a stirred solution of 2,2,6,6-tetramethylpiperidine (2.90 g, 20.495 mmol, 1.00 equiv) in THF (50 mL) was added n-BuLi in hexanes (8.20 mL, 20.495 mmol) dropwise at −78° C. under nitrogen atmosphere. After keep stirring for 1 h at −78° C., 3-chloro-4-(trifluoromethyl)-2-(trimethylsilyl)pyridine (5.2 g, 20.495 mmol) was added into the solution. The resulting mixture was stirred for 2 h at −78° C. Then dry ice (10 g) was added into reaction mixture and warmed to room temperature. The resulting mixture was stirred for 16 h at room temperature. The resulting reaction mixture was concentrated under reduce pressure, the residue was dissolved in 1 M aq. NaOH and reflux in 50° C. for 1 h. Desired product could be detected by LCMS. The mixture was acidified to pH 1 with 1 M HCl (aq.). The resulting mixture was extracted with EA (3×50 mL). The combined organic layers were washed with brine (1×50 mL), dried over anhydrous Na₂SO₄. After filtration, the filtrate was concentrated under reduced pressure to afford 5-chloro-4-(trifluoromethyl)pyridine-3-carboxylic acid (2.5 g). LCMS: (ESI, m/z): [M+1]⁺=226.05. ¹H NMR (300 MHz, DMSO-d₆) δ 9.04 (s, 1H), 8.88 (d, J=0.9 Hz, 1H).

Step 3: To a stirred solution of 5-chloro-4-(trifluoromethyl)pyridine-3-carboxylic acid (2 g, 8.867 mmol) in DCM (10 mL) and MeOH (10 mL) was added TMSCH₂N₂ (2 M in n-hexane, 22.17 mL, 44.335 mmol, 5 equiv) dropwise at 0° C. under nitrogen atmosphere. The resulting mixture was stirred for 2 h at room temperature. Desired product could be detected by LCMS. The resulting mixture was concentrated under reduced pressure to afford methyl 5-chloro-4-(trifluoromethyl)pyridine-3-carboxylate (1.6 g). LCMS: (ESI, m/z): [M+1]⁺=240.00. ¹H NMR (400 MHz, Chloroform-d) δ 8.84 (s, 1H), 8.68-8.64 (m, 1H), 3.97 (s, 3H).

Step 4: To a mixture of methyl 5-chloro-4-(trifluoromethyl)pyridine-3-carboxylate (1.5 g, 6.261 mmol), K₃PO₄ (2.65 g, 12.522 mmol), Pd₂(dba)₃ (573.33 mg, 0.625 mmol, 0.1 equiv) and X-Phos (298.48 mg, 0.625 mmol, 0.1 equiv) in toluene (20 mL) was added 4-chloro-2-fluoroaniline (1.09 g, 7.513 mmol, 1.2 equiv) at room temperature under nitrogen atmosphere. The reaction mixture was stirred for 4 h at 80° C., desired product could be detected by LCMS. The resulting mixture was concentrated under reduced pressure. The resulting mixture was extracted with EA (3× 20 mL). The combined organic layers were washed with brine, dried over anhydrous Na₂SO₄. After filtration, the filtrate was concentrated under reduced pressure. The residue was purified by silica gel column chromatography, eluted with PE/EA (5:1) to afford methyl 5-[(4-chloro-2-fluorophenyl)amino]-4-(trifluoromethyl)pyridine-3-carboxylate (990 mg). LCMS: (ESI, m/z): [M+1]⁺=349.10. ¹H NMR (400 MHz, Chloroform-d) δ 8.57 (s, 1H), 8.31 (s, 1H), 7.25-7.12 (m, 3H), 6.04 (s, 1H), 3.96 (s, 3H).

Step 5: To a stirred solution of methyl 5-[(4-chloro-2-fluorophenyl)amino]-4-(trifluoromethyl)pyridine-3-carboxylate (990 mg, 2.839 mmol) in MeOH (6 mL) and H₂O (6 mL) was added LiOH·H₂O (238.24 mg, 5.678 mmol) in portions at 0° C. The reaction mixture was stirred for 1 h at 60° C., desired product could be detected by LCMS. The reaction mixture was concentrated under reduced pressure. The aqueous layer was acidified to pH 1 with 2 M HCl (aq.). The aqueous layer was extracted with EA (3×30 mL), the combined organic layers and wash with brine (30 mL), dried over anhydrous Na₂SO₄. After filtration, the filtrate was concentrated under reduce pressure. This resulted in 5-[(4-chloro-2-fluorophenyl)amino]-4-(trifluoromethyl)pyridine-3-carboxylic acid (580 mg). LCMS: (ESI, m/z): [M+1]⁺=335.00. ¹H NMR (300 MHz, DMSO-d₆) δ 8.39-8.30 (m, 2H), 8.12 (s, 1H), 7.55-7.47 (m, 1H), 7.31-7.18 (m, 2H).

Step 6: To a stirred solution of 5-[(4-chloro-2-fluorophenyl)amino]-4-(trifluoromethyl)pyridine-3-carboxylic acid (580 mg, 1.733 mmol) and 4-methylmorpholine (350.55 mg, 3.466 mmol) in 1,2-dimethoxyethane (5 mL) was added isobutyl chloroformate (286.94 mg, 2.079 mmol, 1.2 equiv) dropwise at room temperature under nitrogen atmosphere. The reaction mixture was stirred for 1 h at room temperature. Then NaBH₄ (135.17 mg, 3.466 mmol) was added at −15° C., and the resulting mixture was stirred at −15° C. for additional 1 h. Desired product could be detected by LCMS. The reaction was quenched with MeOH at 0° C. The resulting mixture was concentrated under reduced pressure. The residue was purified by reversed-phase flash chromatography with the following conditions: column, C18 silica gel; mobile phase, MeCN in water (10 mmol/L NH₄HCO₃), 5% to 60% gradient in 30 min; detector, UV 254 nm. This resulted in {5-[(4-chloro-2-fluorophenyl)amino]-4-(trifluoromethyl)pyridin-3-yl}methanol (65 mg). LCMS: (ESI, m/z): [M+1]⁺=320.90. ¹H NMR (300 MHz, Chloroform-d) δ 8.53 (d, J=10.2 Hz, 2H), 7.25-7.08 (m, 3H), 6.02 (s, 1H), 4.97-4.91 (m, 2H).

Step 7: To a stirred solution of {5-[(4-chloro-2-fluorophenyl)amino]-4-(trifluoromethyl)pyridin-3-yl}methanol (65 mg, 0.202 mmol) and PPh₃ (79.73 mg, 0.304 mmol, 1.5 equiv) in DCM (1 mL) was added CBr₄ (100.81 mg, 0.304 mmol, 1.5 equiv) at 0° C. under nitrogen atmosphere. The reaction mixture was stirred for 2 h at room temperature. Desired product could be detected by LCMS. The resulting mixture was concentrated under reduced pressure. The residue was purified by Prep-TLC (PE/EA 5:1) to afford 5-(bromomethyl)-N-(4-chloro-2-fluorophenyl)-4-(trifluoromethyl)pyridin-3-amine (45 mg). LCMS: (ESI, m/z): [M+1]⁺=383.1. ¹H NMR (400 MHz, Chloroform-d) δ 8.39 (s, 1H), 8.22 (d, J=1.0 Hz, 1H), 7.17-7.03 (m, 3H), 5.99 (s, 1H), 4.60-4.52 (m, 2H).

Step 8: To a mixture of 5-(bromomethyl)-N-(4-chloro-2-fluorophenyl)-4-(trifluoromethyl)pyridin-3-amine (40 mg, 0.104 mmol) and 2-{[(3,4-dimethylphenyl)methyl]amino}-3-fluoropyridin-4-ylboronic acid (42.88 mg, 0.156 mmol, 1.5 equiv) in dioxane (1 mL) were added K₂CO₃ (43.24 mg, 0.312 mmol, 3 equiv) and Pd(dppf)Cl₂ (7.64 mg, 0.01 mmol, 0.1 equiv). The reaction mixture was stirred for 2 h at 80° C. under nitrogen atmosphere. Desired product could be detected by LCMS. The resulting mixture was filtration and the precipitated solids was washed with EA (3×10 mL). The filtrate was concentrated under reduced pressure. The residue was purified by reversed-phase flash chromatography with the following conditions: column, C18 silica gel; mobile phase, MeCN in water (10 mmol/L NH₄HCO₃), 20% to 95% gradient in 10 min; detector, UV 254 nm. This resulted in N-(4-chloro-2-fluorophenyl)-5-[(2-{[(2,4-dimethoxyphenyl)methyl]amino}-3-fluoropyridin-4-yl)methyl]-4-(trifluoromethyl)pyridin-3-amine (33 mg). LCMS: (ESI, m/z): [M+1]⁺=565.50. ¹H NMR (400 MHz, Chloroform-d) δ 8.41 (s, 1H), 8.02 (s, 1H), 7.74 (d, J=5.3 Hz, 1H), 7.22 (t, J=4.1 Hz, 1H), 7.16-7.00 (m, 3H), 6.46-6.41 (m, 1H), 6.40-6.35 (m, 1H), 6.03 (t, J=5.1 Hz, 1H), 5.95 (s, 1H), 4.56 (t, J=5.9 Hz, 2H), 4.05 (s, 2H), 3.78 (d, J=3.0 Hz, 3H), 3.73 (s, 3H).

Step 9: To a stirred solution of N-(4-chloro-2-fluorophenyl)-5-[(2-{[(2,4-dimethoxyphenyl)methyl]amino}-3-fluoropyridin-4-yl)methyl]-4-(trifluoromethyl)pyridin-3-amine (30 mg, 0.053 mmol) in DCM (0.8 mL) was added TFA (0.2 mL) dropwise at 0° C. under air atmosphere. The reaction mixture was stirred for 1 h at room temperature. Desired product could be detected by LCMS. The resulting mixture was basified to pH 8 with sat. NaHCO₃ (aq.). The resulting mixture was concentrated under reduced pressure. The residue was purified by reversed-phase flash chromatography with the following conditions: column, C18 silica gel; mobile phase, ACN in water (10 mmol/L NH₄HCO₃), 20% to 80% gradient in 25 min; detector, UV 254 nm. This resulted in 5-[(2-amino-3-fluoropyridin-4-yl)methyl]-N-(4-chloro-2-fluorophenyl)-4-(trifluoromethyl)pyridin-3-amine (16 mg). LCMS: (ESI, m/z): [M+1]⁺=415.00. ¹H NMR (400 MHz, Chloroform-d) δ 8.48 (s, 1H), 8.10 (s, 1H), 7.74 (d, J=6.1 Hz, 1H), 7.23-7.02 (m, 3H), 6.20 (d, J=6.7 Hz, 1H), 6.03 (s, 1H), 4.71 (s, 2H), 4.16 (s, 2H). ¹⁹F NMR (377 MHz, Chloroform-d) δ −55.91, −125.23, −145.28.

Step 10: To a stirred solution of 4-({5-[(4-chloro-2-fluorophenyl)amino]-4-(trifluoromethyl)pyridin-3-yl}methyl)-3-fluoropyridin-2-amine (16 mg, 0.038 mmol) and pyridine (30.2 mg, 0.38 mmol, 10 equiv) was added N-methylsulfamoyl chloride (24.6 mg, 0.19 mmol, 5 equiv) in DMA (1 mL) dropwise at 0° C. under air atmosphere. The resulting mixture was stirred for 2 h at room temperature. Desired product could be detected by LCMS. The reaction mixture was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in N-(4-chloro-2-fluorophenyl)-5-({3-fluoro-2-[(methylsulfamoyl)amino]pyridin-4-yl}methyl)-4-(trifluoromethyl)pyridin-3-amine (6.3 mg). LCMS: (ESI, m/z): [M+1]⁺=507.851H NMR (300 MHz, Methanol-d₄) δ 8.18 (s, 1H), 8.10 (s, 1H), 7.96 (d, J=5.2 Hz, 1H), 7.34-7.24 (m, 1H), 7.24-7.10 (m, 2H), 6.61-6.52 (m, 1H), 4.29 (s, 2H), 2.63 (s, 3H). ¹⁹F NMR (282 MHz, Methanol-d₄) δ −58.083, −123.496, −142.339

### Example 39: 3-fluoro-4-[[5-[2-fluoro-3-(trifluoromethoxy) anilino]-4-methyl-3-pyridyl]methyl]-N-(methylsulfamoyl)pyridin-2-amine

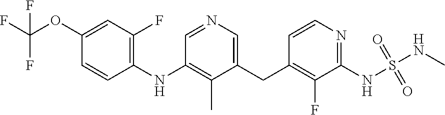 [SMILES: CNS(=O)(=O)Nc1nccc(Cc2cncc(Nc3ccc(OC(F)(F)F)cc3F)c2C)c1F]

### Route

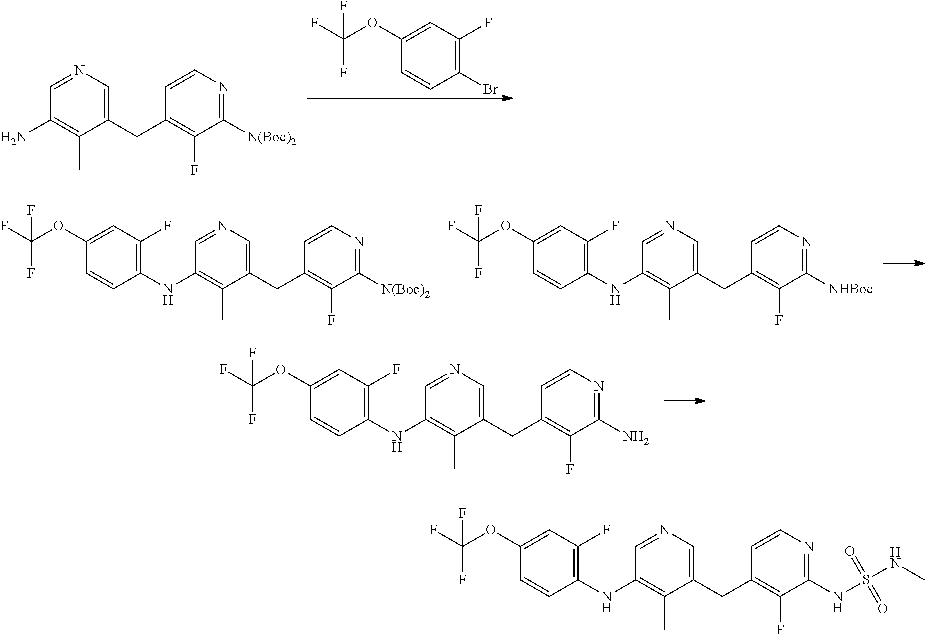

Step 1: A mixture of tert-butyl N-[4-[(5-amino-4-methyl-3-pyridyl)methyl]-3-fluoro-2-pyridyl]-N-tert-butoxycarbonyl-carbamate (200 mg, 462.44 μmol, example 6) and 1-bromo-2-fluoro-4-(trifluoromethoxy)benzene (239.54 mg, 924.88 μmol) and Cs₂CO₃ (452.02 mg, 1.39 mmol), Xantphos (53.52 mg, 92.49 μmol), Pd₂(dba)₃ (42.35 mg, 46.24 μmol) in dioxane (2 mL) under N₂ was stirred at 110° C. for 3 h. The mixture was filtered and the filtrate was concentrated. The residue was purified by flash chromatography on silica gel (Ethyl acetate in Petroleum ether=0-100%) to give tert-butyl N-tert-butoxycarbonyl-N-[3-fluoro-4-[[5-[2-fluoro-4-(trifluoromethoxy) anilino]-4-methyl-3-pyridyl]methyl]-2-pyridyl]carbamate (120 mg, 196.54 μmol) and tert-butyl (3-fluoro-4-((5-((2-fluoro-4-(trifluoromethoxy)phenyl)amino)-4-methylpyridin-3-yl)methyl)pyridin-2-yl) carbamate (150 mg, 293.86 μmol). ¹H NMR (400 MHz, DMSO-d₆) δ=8.24 (d, J=5.2 Hz, 1H), 8.19-8.06 (m, 1H), 7.76 (s, 1H), 7.45-7.34 (m, 1H), 7.32-7.19 (m, 2H), 7.10-6.98 (m, 1H), 6.66 (t, J=9.2 Hz, 1H), 4.18 (s, 2H), 1.99 (s, 3H), 1.33 (s, 18H). ¹⁹F NMR (376.5 MHz, DMSO-d₆) δ=−57.472 ppm.

Step 2: N-tert-butoxycarbonyl-N-[3-fluoro-4-[[5-[2-fluoro-4-(trifluoromethoxy) anilino]-4-methyl-3-pyridyl]methyl]-2-pyridyl]carbamate (100 mg, 163.78 μmol) and HCl/MeOH (4 M, 2.20 mL) in MeOH (0.3 mL) under N₂ was stirred at 25° C. for 24 h. The mixture was added into NH₃/MeOH (7 M, 10 mL) dropwise, then concentrated. The residue was purified by flash chromatography on silica gel (MeOH in DCM=0-10%) to give 3-fluoro-4-[[5-[2-fluoro-4-(trifluoromethoxy) anilino]-4-methyl-3-pyridyl]methyl]pyridin-2-amine (60 mg, 146.22 μmol). ¹H NMR (400 MHz, CDCl₃) δ=8.40 (s, 1H), 8.21 (s, 1H), 7.73 (d, J=5.2 Hz, 1H), 7.04 (d, J=11.2 Hz, 1H), 6.90 (d, J=9.2 Hz, 1H), 6.78 (t, J=8.8 Hz, 1H), 6.28 (t, J=4.8 Hz, 1H), 5.48 (s, 1H), 4.82 (s, 2H), 4.01 (s, 2H), 2.16 (s, 3H). ¹⁹F NMR (376.5 MHz, CDCl₃) δ=−58.526 ppm, −129.203 ppm, −144.864 ppm.

Step 3: To a solution of 3-fluoro-4-[[5-[2-fluoro-4-(trifluoromethoxy) anilino]-4-methyl-3-pyridyl]methyl]pyridin-2-amine (100 mg, 243.70 μmol) and Py (192.77 mg, 2.44 mmol, 196.70 μL) in MeCN (0.9 mL) was added N-methylsulfamoyl chloride (157.88 mg, 1.22 mmol) under N₂. The mixture was stirred at 25° C. for 16 h. The mixture was concentrated. The residue was purified by prep-HPLC (column: Boston Prime C18 150*30 mm*5 μm; mobile phase: [water(NH₃H₂O+NH₄HCO₃)−ACN]; B %: 28%-58%, 7 min) to give 3-fluoro-4-[[5-[2-fluoro-3-(trifluoromethoxy) anilino]-4-methyl-3-pyridyl]methyl]-N-(methylsulfamoyl)pyridin-2-amine (15.3 mg, 30.39 μmol). ¹H NMR (400 MHz, DMSO-d₆) δ=8.09 (d, J=3.6 Hz, 2H), 7.94 (d, J=5.2 Hz, 1H), 7.73 (s, 1H), 7.34 (dd, J=1.6, 11.2 Hz, 1H), 7.04 (d, J=9.6 Hz, 1H), 6.75-6.62 (m, 2H), 4.06 (s, 2H), 2.47 (s, 3H), 2.05 (s, 3H). ¹⁹F NMR (376.5 MHz, DMSO-d₆) δ=−57.451 ppm, −124.486 ppm, 138.407 ppm. LCMS Rₜ=1.198 min in 3 min chromatography, 10-80CD, ESI calcd. for C₂₀H₁₉F₅N₅O₃S [M+H]⁺504.1, found 504.2.

### Example 40: 4-[[6-(4-chloro-2-fluoro-phenoxy) pyrazin-2-yl]methyl]-3-fluoro-N-(methylsulfamoyl)pyridin-2-amine

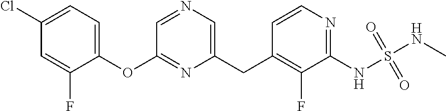 [SMILES: CNS(=O)(=O)Nc1nccc(Cc2cncc(Oc3ccc(Cl)cc3F)n2)c1F]

### Route

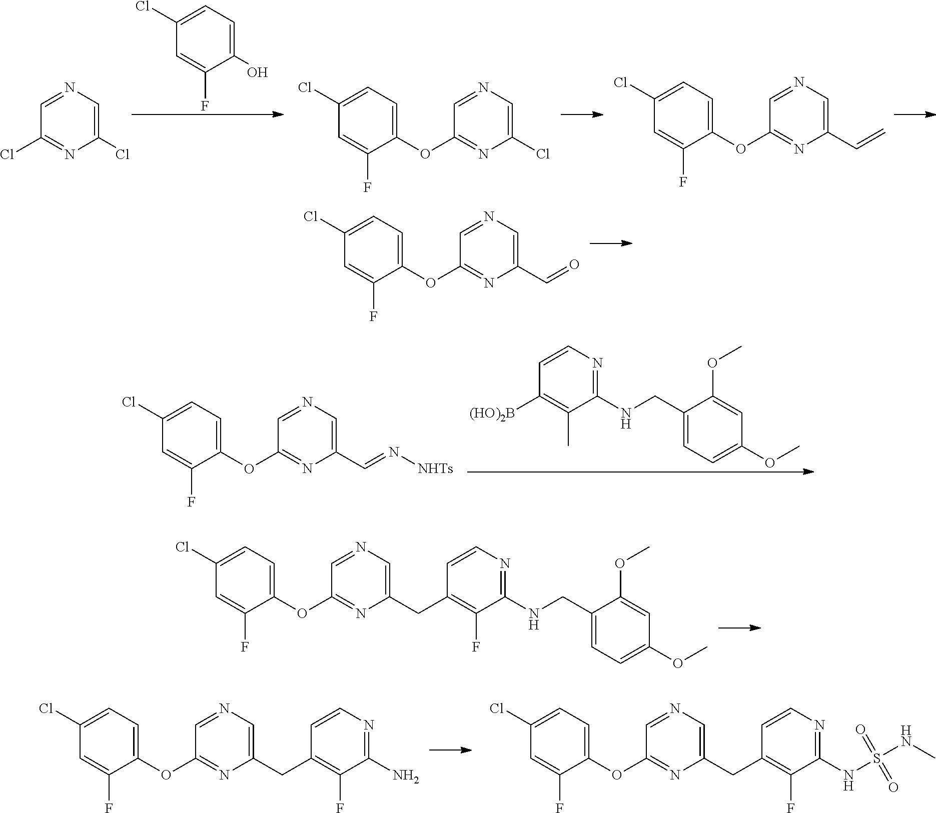

Step 1: To a solution of 2,6-dichloropyrazine (4.32 g, 28.99 mmol) and 4-chloro-2-fluoro-phenol (3.54 g, 24.16 mmol, 2.57 mL) in DMSO (50 mL) was added K₃PO₄ (10.26 g, 48.31 mmol). The mixture was stirred at 60° C. for 5 hr. The mixture was poured into sat. NH₄Cl (50 mL) and extracted with EtOAc (50 mL×3). The combined organic layers were washed with brine (50 mL), dried over anhydrous Na₂SO₄, filtered and concentrated under reduced pressure to give a residue. The crude was purified by flash chromatography on silica gel (Dichloromethane in Petroleum ether=0 to 50%) to give 2-chloro-6-(4-chloro-2-fluoro-phenoxy) pyrazine (6 g, 23.16 mmol). ¹H NMR (400 MHz, DMSO-d₆) δ=8.71 (s, 1H), 8.59 (s, 1H), 7.72 (dd, J=2.4, 10.4 Hz, 1H), 7.53 (t, J=8.8 Hz, 1H), 7.44-7.37 (m, 1H). ¹⁹F NMR (376.5 MHz, DMSO-d₆) δ=−125.394 ppm.

Step 2: To a solution of 2-chloro-6-(4-chloro-2-fluoro-phenoxy) pyrazine (2 g, 7.72 mmol) and 4,4,5,5-tetramethyl-2-vinyl-1,3,2-dioxaborolane (1.78 g, 11.58 mmol, 1.96 mL) in dioxane (25 mL) and H₂O (5 mL) were added Pd(dppf)Cl₂ (282.44 mg, 386.01 μmol) and Na₂CO₃ (2.45 g, 23.16 mmol). The mixture was stirred at 100° C. for 12 hr. The mixture was poured into sat. NH₄Cl (25 mL) and extracted with EtOAc (25 mL×3). The combined organic layers were washed with brine (25 mL), dried over anhydrous Na₂SO₄, filtered and concentrated under reduced pressure to give a residue. The crude was purified by flash chromatography on silica gel (Dichloromethane in Petroleum ether=0 to 50%) to give 2-(4-chloro-2-fluoro-phenoxy)-6-vinyl-pyrazine (1.6 g, 6.38 mmol). ¹H NMR (400 MHz, CDCl₃) δ=8.33 (s, 1H), 8.24 (s, 1H), 7.26-7.16 (m, 3H), 6.64 (dd, J=10.8, 17.2 Hz, 1H), 6.09 (d, J=17.2 Hz, 1H), 5.49 (d, J=10.8 Hz, 1H). ¹⁹F NMR (376.5 MHz, CDCl₃) δ=−123.866 ppm.

Step 3: To a solution of 2-(4-chloro-2-fluoro-phenoxy)-6-vinyl-pyrazine (1.6 g, 6.38 mmol) in THF (20 mL) and H₂O (2.5 mL) were added NaIO₄ (5.46 g, 25.53 mmol, 1.41 mL) and K₂OsO₄·2H₂O (235.19 mg, 638.33 umol). The mixture was stirred at 25° C. for 2 hr. The resulting solution was diluted water (10 mL) and quenched with Saturated Na₂SO₃ solution (20 mL) until KI test paper turn to white. The mixture extracted with ethyl acetate (10 mL×3). The organic layers were combined, dried over sodium sulfate, filtered and concentrated. The crude was purified by flash chromatography on silica gel (Dichloromethane in Petroleum ether=0 to 50%) to give 6-(4-chloro-2-fluoro-phenoxy) pyrazine-2-carbaldehyde (1.5 g, 5.94 mmol). ¹H NMR (400 MHz, CDCl₃) δ=9.85 (s, 1H), 8.89 (s, 1H), 8.72 (s, 1H), 7.29-7.22 (m, 3H). ¹⁹F NMR (376.5 MHz, CDCl₃) δ=−123.641 ppm.

Step 4: To a solution of 6-(4-chloro-2-fluoro-phenoxy) pyrazine-2-carbaldehyde (1.6 g, 6.33 mmol) in THF (15 mL) was added 4-methylbenzenesulfonohydrazide (1.30 g, 6.97 mmol). The mixture was stirred at 60° C. for 4 hr. The mixture was concentrated. The crude product was triturated with MeOH (5 mL) and purified by Prep-HPLC (column: Xtimate C18 150×40 mm×10 um; mobile phase: [water(FA)−ACN]; B %: 52%-82%, 7 min) to give N-[(E)-[6-(4-chloro-2-fluoro-phenoxy) pyrazin-2-yl]methyleneamino]-4-methyl-benzenesulfonamide (1.9 g, 4.51 mmol). ¹H NMR (400 MHz, CDCl₃) δ=11.84 (s, 1H), 8.56 (s, 1H), 8.35 (s, 1H), 7.65 (d, J=8.0 Hz, 2H), 7.41-7.37 (m, 4H), 7.26-7.20 (m, 2H), 2.40 (s, 3H). ¹⁹F NMR (376.5 MHz, CDCl₃) δ=−124.630 ppm.

Step 5: To a solution of N-[(E)-[6-(4-chloro-2-fluoro-phenoxy) pyrazin-2-yl]methyleneamino]-4-methyl-benzenesulfonamide (300 mg, 712.85 mmol) and [2-[(2,4-dimethoxyphenyl)methylamino]-3-fluoro-4-pyridyl]boronic acid (654.60 mg, 2.14 mmol, example 30) in dioxane (2 mL) was added K₂CO₃ (295.56 mg, 2.14 mmol). The mixture was stirred at 110° C. for 4 hr. The mixture was concentrated. The crude was purified by flash chromatography on silica gel (Ethyl acetate in Petroleum ether=0 to 50%) and purified by flash chromatography on silica gel (Ethyl acetate in Petroleum ether=0 to 50%) to give 4-[[6-(4-chloro-2-fluoro-phenoxy) pyrazin-2-yl]methyl]-N-[(2,4-dimethoxyphenyl)methyl]-3-fluoro-pyridin-2-amine (160 mg, 320.70 mmol). LCMS Rₜ=0.851 min 1.5 min chromatography, 5-95AB, ESI calcd. for C₂₅H₂₂ClF₂N₄O₃ [M+H]⁺499.1, found 499.1.

Step 6: To a solution of 4-[[6-(4-chloro-2-fluoro-phenoxy) pyrazin-2-yl]methyl]-N-[(2,4-dimethoxyphenyl)methyl]-3-fluoro-pyridin-2-amine (160 mg, 320.70 mmol) was added TFA (1 mL). The mixture was stirred at 25° C. for 1 hr. The mixture was added dropwise into H₂O (3 mL) at 0° C. The mixture was adjusted to pH=7 with saturated NaHCO₃ solution slowly at 0° C. and the aqueous layer was extracted with EtOAc (5 mL×2). The combined organic layer was washed with brine (5 mL), dried over anhydrous Na₂SO₄, filtered and concentrated. The crude was purified by flash chromatography on silica gel (Ethyl acetate in Petroleum ether=0 to 50%) to give 4-[[6-(4-chloro-2-fluoro-phenoxy) pyrazin-2-yl]methyl]-3-fluoro-pyridin-2-amine (50 mg, 143.38 umol). LCMS Rₜ=1.42 min 3.0 min chromatography, 5-95CD, ESI calcd. for C₁₆H₁₂ClF₂N₄O [M+H]⁺349.1, found 349.0.

Step 7: To a solution of 4-[[6-(4-chloro-2-fluoro-phenoxy) pyrazin-2-yl]methyl]-3-fluoro-pyridin-2-amine (40 mg, 114.70 mmol) and N-methylsulfamoyl chloride (74.31 mg, 573.50 mmol) in MeCN (1 mL) was added Py (90.73 mg, 1.15 mmol, 92.58 mL). The mixture was stirred at 25° C. for 1 hr. The mixture was concentrated. The crude was purified by Prep-HPLC (column: Boston Prime C18150×30 mm×5 um; mobile phase: [water(NH₃H₂O+NH₄HCO₃)−ACN]; B %: 32%-62%, 7 min) to give 4-[[6-(4-chloro-2-fluoro-phenoxy) pyrazin-2-yl]methyl]-3-fluoro-N-(methylsulfamoyl)pyridin-2-amine (4.9 mg, 11.09 umol). ¹H NMR (400 MHz, CDCl₃) δ=8.37 (s, 1H), 8.24 (s, 1H), 7.91 (d, J=4.8 Hz, 1H), 7.21-7.09 (m, 4H), 6.71 (t, J=5.2 Hz, 1H), 5.48 (br s, 1H), 3.99 (s, 2H), 2.72 (d, J=5.2 Hz, 3H). ¹⁹F NMR (376.5 MHz, CDCl₃) δ=−123.484, −143.314 ppm. LCMS Rₜ=0.881 min 1.5 min chromatography, 5-95AB, ESI calcd. for C₁₇H₁₅ClF₂N₅O₃S [M+H]⁺442.1, found 441.9.

### Example 41: 7-fluoro-N-[5-[[3-fluoro-2-(methylsulfamoylamino)-4-pyridyl]methyl]-4-methyl-3-pyridyl]-2,3-dihydro-[1,4]dioxino[2,3-b]pyridin-6-amine

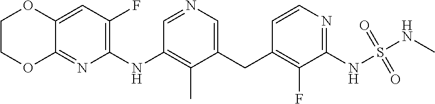 [SMILES: CNS(=O)(=O)Nc1nccc(Cc2cncc(Nc3nc4c(cc3F)OCCO4)c2C)c1F]

### Route

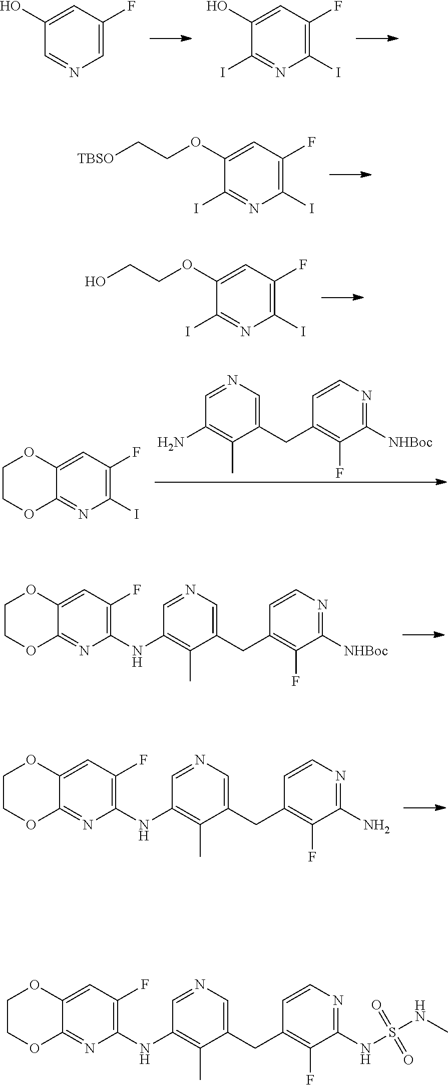

Step 1: To a solution of 5-fluoropyridin-3-ol (2 g, 17.69 mmol) in Na₂CO₃ (30 mL) and H₂O (10 mL) was added I₂ (9.20 g, 36.25 mmol, 7.30 mL). The mixture was stirred at 25° C. for 18 hr. The mixture was poured into sat. Na₂SO₃ (10 mL) to adjust to pH=5. The aqueous layer was extracted with EtOAc (50 mL×3). The combined organic phase was washed with brine (50 mL), dried over anhydrous Na₂SO₄, filtered and concentrated under reduced pressure to give 5-fluoro-2,6-diiodo-pyridin-3-ol (6.4 g, 17.54 mmol). ¹H NMR (400 MHz, CDCl₃) δ=6.95 (d, J=7.6 Hz, 1H), 5.76 (br s, 1H)

Step 2: To a solution of 5-fluoro-2,6-diiodo-pyridin-3-ol (5 g, 13.70 mmol) in DMF (50 mL) were added CS₂CO₃ (13.39 g, 41.11 mmol) and 2-bromoethoxy-tert-butyl-dimethyl-silane (3.61 g, 15.07 mmol). The mixture was stirred at 80° C. for 3 hr. The resulting mixture was filtered, and the filter cake was washed with DCM (50 mL×3). The filtrate was concentrated. The crude was purified by flash chromatography on silica gel (EtOAc in PE=10%) to give tert-butyl-[2-[(5-fluoro-2,6-diiodo-3-pyridyl)oxy]ethoxy]-dimethyl-silane (4.7 g, 8.98 mmol). ¹H NMR (400 MHz, CDCl₃) δ=6.88 (d, J=8.4 Hz, 1H), 4.16-4.11 (m, 2H), 4.04-3.99 (m, 2H), 0.89 (s, 9H), 0.13 (s, 6H).

Step 3: A solution of tert-butyl-[2-[(5-fluoro-2,6-diiodo-3-pyridyl)oxy]ethoxy]-dimethyl-silane (4.5 g, 8.60 mmol) in HCl/MeOH (4 M, 22.50 mL) was stirred at 25° C. for 2 h. The mixture was concentrated. The mixture was added to NH₃/MeOH (3 mL) to adjust pH=7. The mixture was concentrated. The mixture was purified by flash chromatography on silica gel (EtOAc in petroleum ether=0-10%) to afford 2-[(5-fluoro-2,6-diiodo-3-pyridyl)oxy]ethanol (2.9 g, 7.09 mmol). ¹H NMR (400 MHz, DMSO-d₆) δ=7.50 (d, J=9.6 Hz, 1H), 4.94 (t, J=5.2 Hz, 1H), 4.14 (t, J=5.2 Hz, 2H), 3.74 (q, J=5.2 Hz, 2H).

Step 4: To a solution of 2-[(5-fluoro-2,6-diiodo-3-pyridyl)oxy]ethanol (1.7 g, 4.16 mmol) in t-BuOH (24 mL) was added t-BuOK (559.78 mg, 4.99 mmol). The mixture was stirred at 90° C. for 3 h. The mixture was poured into water (20 mL). The aqueous layer was extracted with EtOAc (30 mL×3). The combined organic phase was washed with brine (20 mL), dried over anhydrous Na₂SO₄, filtered and concentrated under reduced pressure. The mixture was purified by flash chromatography on silica gel (DCM in petroleum ether=0-100%) to afford 7-fluoro-6-iodo-2,3-dihydro-[1,4]dioxino[2,3-b]pyridine (450 mg, 1.60 mmol). 1H NMR (400 MHz, DMSO-d₆) δ=7.44 (d, J=7.6 Hz, 1H), 4.46-4.36 (m, 2H), 4.31-4.23 (m, 2H)

Step 5:7-fluoro-6-iodo-2,3-dihydro-[1,4]dioxino[2,3-b]pyridine (228.29 mg, 812.34 μmol) and Pd₂(dba)₃ (24.80 mg, 27.08 μmol) were added under nitrogen to a solution of tert-butyl N-[4-[(5-amino-4-methyl-3-pyridyl)methyl]-3-fluoro-2-pyridyl]carbamate (180 mg, 541.56 umol) in dioxane (5 mL). The medium was degassed for 5 minutes under N₂ before adding Cs₂CO₃ (247.03 mg, 758.19 μmol) and Xantphos (31.34 mg, 54.16 μmol). The reaction medium was stirred at 80° C. for 2 hours. The mixture was concentrated. The mixture was purified by flash chromatography on silica gel (DCM in petroleum ether=0-100%) to afford tert-butyl N-[3-fluoro-4-[[5-[(7-fluoro-2,3-dihydro-[1,4]dioxino[2,3-b]pyridin-6-yl)amino]-4-methyl-3-pyridyl]methyl]-2-pyridyl]carbamate (200 mg, 411.96 μmol). ¹H NMR (400 MHz, CDCl₃) δ=8.90 (s, 1H), 8.22-8.01 (m, 2H), 7.04 (d, J=10.0 Hz, 1H), 6.93 (br s, 1H), 6.62 (t, J=5.2 Hz, 1H), 5.96 (br s, 1H), 4.39-4.34 (m, 2H), 4.21-4.18 (m, 2H), 4.05 (s, 2H), 2.11 (s, 3H), 1.53 (s, 9H).

Step 6: A solution of tert-butyl N-[3-fluoro-4-[[5-[(7-fluoro-2,3-dihydro-[1,4]dioxino[2,3-b]pyridin-6-yl)amino]-4-methyl-3-pyridyl]methyl]-2-pyridyl]carbamate (200 mg, 411.96 umol) in HCl/MeOH (4 mL) was stirred at 25° C. for 3 h. The mixture was concentrated. The mixture was added to NH₃/MeOH (3 mL) to adjust pH=7. The mixture was purified by flash chromatography on silica gel (MeOH in DCM=0-10%) to afford N-[5-[(2-amino-3-fluoro-4-pyridyl)methyl]-4-methyl-3-pyridyl]-7-fluoro-2,3-dihydro-[1,4]dioxino[2,3-b]pyridin-6-amine (110 mg, 285.44 μmol). LCMS Rt=0.612 min in 1.5 min chromatography, 5-95AB, ESI calcd. for C₁₉H₁₈F₂N₅O₂ [M+H]⁺386.1, found 386.1.

Step 7: To a solution of N-[5-[(2-amino-3-fluoro-4-pyridyl)methyl]-4-methyl-3-pyridyl]-7-fluoro-2,3-dihydro-[1,4]dioxino[2,3-b]pyridin-6-amine (60 mg, 155.70 μmol) in MeCN (1 mL) were added Py (123.16 mg, 1.56 mmol, 125.67 μL) and methylsulfamoyl chloride (100.86 mg, 778.48 μmol) at 25° C. The mixture was stirred at 25° C. for 24 h. The mixture was concentrated. The mixture was purified by Prep-HPLC (column: Phenomenex C18 75×30 mm×3 um; mobile phase: [water(NH₃H₂O+NH₄HCO₃)−ACN]; B %: 15%-45%, 8 min) to give 7-fluoro-N-[5-[[3-fluoro-2-(methylsulfamoylamino)-4-pyridyl]methyl]-4-methyl-3-pyridyl]-2,3-dihydro-[1,4]dioxino[2,3-b]pyridin-6-amine (36.2 mg, 75.66 μmol) ¹H NMR (400 MHz, CDCl₃) δ=8.89 (br s, 1H), 8.15 (br s, 1H), 7.93 (d, J=5.2 Hz, 1H), 7.05 (d, J=10.0 Hz, 1H), 6.58 (t, J=5.2 Hz, 1H), 5.97 (br s, 1H), 5.52 (br s, 1H), 4.46-4.27 (m, 2H), 4.25-4.10 (m, 2H), 4.04 (s, 2H), 2.76 (s, 3H), 2.04 (s, 3H). ¹⁹F NMR (376.5 MHz, CDCl₃) δ=−142.641, −145.030 ppm. LCMS Rt=0.709 min in 1.5 min chromatography, 5-95AB, ESI calcd. for C₂₀H₂₁F₂N₆O₄S [M+H]⁺479.1, found 479.2.

### Example 42: 3-fluoro-4-[[5-[(6-fluoro-2,3-dihydro-1,4-benzodioxin-7-yl)amino]-4-methyl-3-pyridyl]methyl]-N-(methylsulfamoyl)pyridin-2-amine

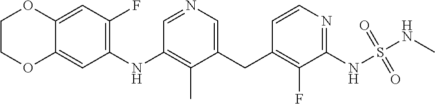 [SMILES: CNS(=O)(=O)Nc1nccc(Cc2cncc(Nc3cc4c(cc3F)OCCO4)c2C)c1F]

### Route

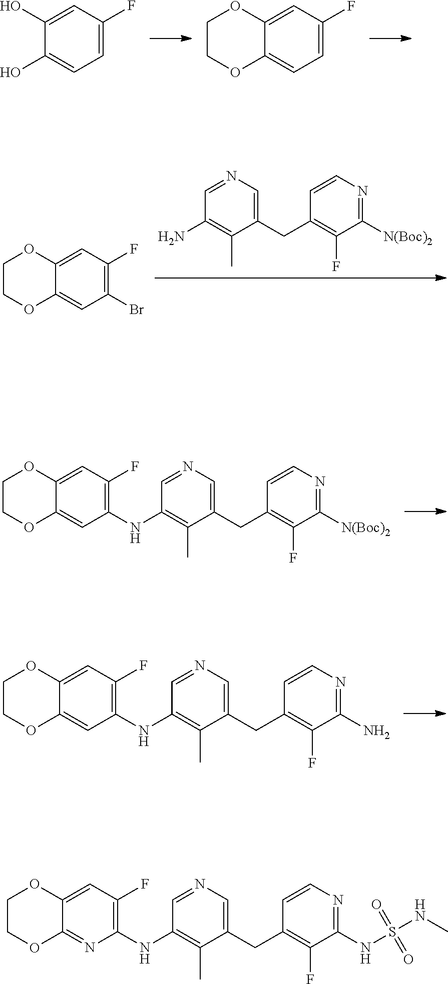

Step 1: To a solution of 4-fluorobenzene-1,2-diol (2 g, 15.61 mmol) in DMF (20 mL) were added 1,2-dibromoethane (7.33 g, 39.03 mmol, 2.94 mL) and K₂CO₃ (8.63 g, 62.45 mmol). The mixture was stirred at 80° C. for 2 hr. Water (100 mL) was added and the mixture were extracted with EtOAc (80 mL×2). The organic layer was dried over anhydrous Na₂SO₄, filtered and concentrated to give 6-fluoro-2,3-dihydro-1,4-benzodioxine (1.3 g, 8.43 mmol). 1H NMR (400 MHz, DMSO-d₆) δ=6.85 (dd, J=6.0, 9.2 Hz, 1H), 6.73 (dd, J=3.2, 10.0 Hz, 1H), 6.66-6.61 (m, 1H), 4.25-4.19 (m, 4H). ¹⁹F NMR (376.5 MHz, DMSO-d₆) δ=−121.602.

Step 2: To a solution of 6-fluoro-2,3-dihydro-1,4-benzodioxine (1.3 g, 8.43 mmol) in MeCN (20 mL) were added NBS (2.25 g, 12.65 mmol) and TFA (96.17 mg, 843.40 umol, 62.45 mL). The mixture was stirred at 25° C. for 16 hr. Water (50 mL) was added and the mixture were extracted with EtOAc (30 mL×2). The organic layer was dried over anhydrous Na₂SO₄, filtered and concentrated to give 6-bromo-7-fluoro-2,3-dihydro-1,4-benzodioxine (1.7 g, 7.30 mmol) ¹H NMR (400 MHz, CDCl₃) δ=7.03 (d, J=6.4 Hz, 1H), 6.68 (d, J=9.2, Hz, 1H), 4.26-4.21 (m, 4H). ¹⁹F NMR (376.5 MHz, CDCl₃) δ=−116.066.

Step 3: To a solution of 6-bromo-7-fluoro-2,3-dihydro-1,4-benzodioxine (50 mg, 214.56 mmol) in dioxane (1 mL) were added tert-butyl N-[4-[(5-amino-4-methyl-3-pyridyl)methyl]-3-fluoro-2-pyridyl]-N-tert-butoxycarbonyl-carbamate (92.79 mg, 214.56 mmol, example 6), Cs₂CO₃ (139.82 mg, 429.12 mmol), Pd₂(dba)₃ (9.82 mg, 10.73 umol) and Xantphos (12.41 mg, 21.46 mmol). The mixture was stirred at 100° C. for 4 hr. Water (20 mL) was added and the mixture were extracted with EtOAc (20 mL×2). The organic layer was dried over anhydrous Na₂SO₄, filtered and concentrated. The crude was purified by flash column chromatography on silica gel (EtOAc in petroleum ether=0-80%) to give tert-butyl N-tert-butoxycarbonyl-N-[3-fluoro-4-[[5-[(6-fluoro-2,3-dihydro-1,4-benzodioxin-7-yl)amino]-4-methyl-3-pyridyl]methyl]-2-pyridyl]carbamate (70 mg, 119.74 mmol). LCMS Rₜ=0.818 min in 1.5 min chromatography, 5-95AB, ESI calcd. for C₃₀H₃₅F₂N₄O₆ [M+H]⁺585.2, found 585.3.

Step 4: To a solution of tert-butyl N-tert-butoxycarbonyl-N-[3-fluoro-4-[[5-[(6-fluoro-2,3-dihydro-1,4-benzodioxin-7-yl)amino]-4-methyl-3-pyridyl]methyl]-2-pyridyl]carbamate (70 mg, 119.74 mmol) in DCM (1 mL) was added HCl/MeOH (3 mL). The mixutre was stirred at 25° C. for 1 hr. The mixture was adjusted to Ph=9 with NH₃·MeOH (7 M, 10 mL) and concentrated. The organic layer was dried over anhydrous Na₂SO₄, filtered and concentrated. The residue was purified by flash column chromatography on silica gel (EtOAc in petroleum ether=0-30%) to give 3-fluoro-4-[[5-[(6-fluoro-2,3-dihydro-1,4-benzodioxin-7-yl)amino]-4-methyl-3-pyridyl]methyl]pyridin-2-amine (30 mg, 78.05 mmol). ¹H NMR (400 MHz, CDCl₃) δ=8.25 (s, 1H), 8.08 (s, 1H), 7.73 (d, J=5.2 Hz, 1H), 6.69 (dd, J=4.0, 11.6 Hz, 1H), 6.49 (d, J=8.0 Hz, 1H), 6.26 (m, 1H), 4.23-4.21 (m, 4H), 3.98 (s, 2H), 2.14 (s, 3H). ¹⁹F NMR (376.5 MHz, CDCl₃) δ=−138.566, −145.341.

Step 5: To a solution of 3-fluoro-4-[[5-[(6-fluoro-2,3-dihydro-1,4-benzodioxin-7-yl)amino]-4-methyl-3-pyridyl]methyl]pyridin-2-amine (20 mg, 52.03 mmol) in MeCN (1 mL) were added N-methylsulfamoyl chloride (33.71 mg, 260.16 mmol) and Py (41.16 mg, 520.32 mmol, 42.00 mL). The mixture was stirred at 40° C. for 1 hr. The mixture was concentrated and purified by Pre-HPLC (column: Boston Prime C18 150*30 mm*5 um; mobile phase: [water(NH3H2O+NH4HCO3)−ACN]; B %: 22%-52%, 7 min) to give 3-fluoro-4-[[5-[(6-fluoro-2,3-dihydro-1,4-benzodioxin-7-yl)amino]-4-methyl-3-pyridyl]methyl]-N-(methylsulfamoyl)pyridin-2-amine (3.1 mg, 6.49 umol). ¹H NMR (400 MHz, DMSO-d₆) δ=10.38 (brs, 1H), 7.98 (d, J=4.8 Hz, 1H), 7.92 (s, 1H), 7.79 (d, J=2.0 Hz, 1H), 7.13 (s, 1H), 7.00-6.99 (m, 1H), 6.84 (brs, 1H), 6.72 (s, 1H), 6.45 (d, J=8.0 Hz, 1H), 4.21-4.19 (m, 4H), 4.05 (s, 2H), 2.49 (s, 3H), 2.07 (s, 3H). ¹⁹F NMR (376.5 MHz, DMSO-d₆) δ=−131.509, −138.306. LCMS Rₜ=0.636 min in 1.5 min chromatography, 5-95AB, ESI calcd. for C₂₁H₂₂F₂N₅O₄S [M+H]⁺478.1, found 478.1.

### Example 43: 4-({5-[(4-chloro-2-fluorophenyl)amino]-4-methylpyridin-3-yl}methyl)-N-methyl-2H,3H-pyrrolo[2,3-b]pyridine-1-sulfonamide

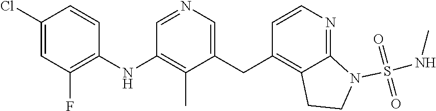 [SMILES: CNS(=O)(=O)N1CCc2c(Cc3cncc(Nc4ccc(Cl)cc4F)c3C)ccnc21]

### Route

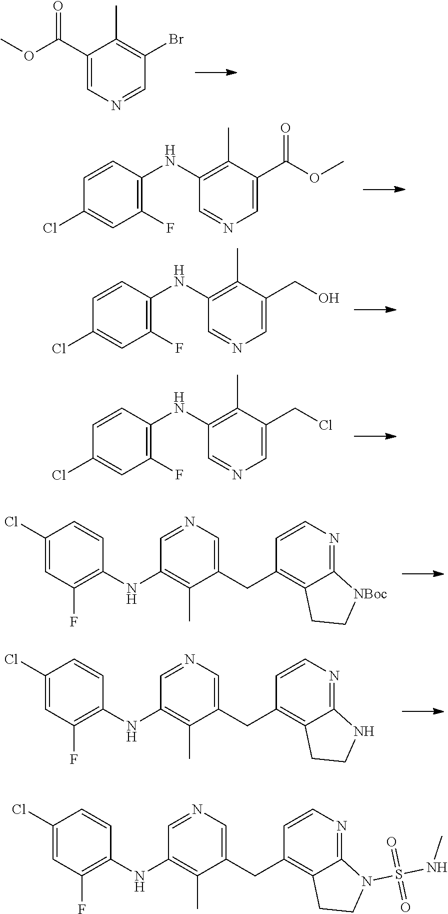

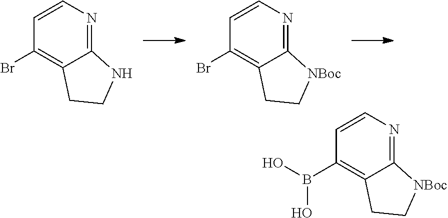

Step 1: To a stirred mixture of methyl 5-bromo-4-methylpyridine-3-carboxylate (500 mg, 2.173 mmol, example 2) and 4-chloro-2-fluoroaniline (379.62 mg, 2.608 mmol, 1.2 equiv) in dioxane (10 mL) were added Cs₂CO₃ (2.13 g, 6.519 mmol, 3.0 equiv), RuPhos Pd G3 (181.77 mg, 0.217 mmol, 0.1 equiv) and RuPhos (101.42 mg, 0.217 mmol, 0.1 equiv) at room temperature under nitrogen atmosphere. The resulting mixture was stirred for 2 h at 80° C. under nitrogen atmosphere. Desired product could be detected by LCMS. The reaction mixture was concentrated under reduced pressure. The residue was purified by silica gel column chromatography, eluted with PE/EA (5:1) to afford methyl 5-[(4-chloro-2-fluorophenyl)amino]-4-methylpyridine-3-carboxylate (573 mg). LCMS: (ESI, m/z): [M+1]⁺=295.00.

Step 2: To a stirred mixture of methyl 5-[(4-chloro-2-fluorophenyl)amino]-4-methylpyridine-3-carboxylate (473 mg, 1.605 mmol) and CaCl₂) (890.60 mg, 8.025 mmol, 5.0 equiv) in MeOH (5 mL) was added NaBH₄ (607.16 mg, 16.050 mmol, 10.0 equiv) in portions at 0° C. under nitrogen atmosphere. The resulting mixture was stirred for 1 h at 0° C. under nitrogen atmosphere. Desired product could be detected by LCMS. The reaction was quenched by the addition of sat. NH₄Cl (aq.) (50 mL) at 0° C. The resulting mixture was extracted with EA (3×50 mL). The combined organic layers were dried over anhydrous Na₂SO₄. After filtration, the filtrate was concentrated under reduced pressure. The residue was purified by silica gel column chromatography, eluted with CH₂Cl₂/MeOH (10:1) to afford {5-[(4-chloro-2-fluorophenyl)amino]-4-methylpyridin-3-yl}methanol (470 mg). LCMS: (ESI, m/z): [M+1]⁺=267.00. ¹H NMR (400 MHz, Chloroform-d) δ 8.39 (s, 1H), 8.32 (s, 1H), 7.19-7.12 (m, 1H), 7.03-6.95 (m, 1H), 6.74 (t, J=8.8 Hz, 1H), 5.47 (s, 1H), 4.78 (s, 2H), 2.30 (s, 3H).

Step 3: To a stirred solution of {5-[(4-chloro-2-fluorophenyl)amino]-4-methylpyridin-3-yl}methanol (100 mg, 0.375 mmol) in DCM (2 mL) was added SOCl₂ (89.21 mg, 0.750 mmol) dropwise at 0° C. The resulting mixture was stirred for 16 h at 50° C. under nitrogen atmosphere. Desired product could be detected by LCMS. The resulting mixture was concentrated under reduced pressure. This resulted in N-(4-chloro-2-fluorophenyl)-5-(chloromethyl)-4-methylpyridin-3-amine (80 mg). LCMS: (ESI, m/z): [M+1]⁺=285.10. ¹H NMR (400 MHz, Chloroform-d) δ 8.42 (s, 1H), 8.31 (s, 1H), 7.17-7.13 (m, 1H), 7.02-6.97 (m, 1H), 6.82-6.73 (m, 1H), 5.47 (s, 1H), 4.65 (s, 2H), 2.35 (s, 3H).

Step 4: To a stirred solution of 4-bromo-1H, 2H, 3H-pyrrolo[2,3-b]pyridine (450 mg, 2.261 mmol) and TEA (686.31 mg, 6.783 mmol, 3 equiv) in DCM (5 mL) were added (Boc)₂O (740.10 mg, 3.392 mmol, 1.5 equiv) and DMAP (27.62 mg, 0.226 mmol, 0.1 equiv) at 0° C. After stirring for 4 h at room temperature under nitrogen atmosphere. Desired product could be detected by LCMS. The resulting mixture was extracted with DCM (3×20 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (3:1) to afford tert-butyl 4-bromo-2H,3H-pyrrolo[2,3-b]pyridine-1-carboxylate (475 mg). LCMS: (ESI, m/z): [M+1]⁺=299.10. ¹H NMR (400 MHz, Chloroform-d) δ 8.07-8.01 (m, 1H), 6.98 (d, J=5.6 Hz, 1H), 4.08-3.99 (m, 2H), 3.09-2.99 (m, 2H), 1.56 (s, 9H).

Step 5: To a solution of tert-butyl 4-bromo-2H,3H-pyrrolo[2,3-b]pyridine-1-carboxylate (300 mg, 1.0 mmol) and 4,4,5,5-tetramethyl-2-(tetramethyl-1,3,2-dioxaborolan-2-yl)-1,3,2-dioxaborolane (305.57 mg, 1.20 mmol) in dioxane (5 mL) were added AcOK (196.83 mg, 2.006 mmol) and Pd(PPh₃)₂Cl₂ (70.39 mg, 0.100 mmol, 0.1 equiv). After stirring for 2 h at 80° C. under nitrogen atmosphere, the resulting mixture was concentrated under reduced pressure. The residue was treated with formic acid (2 mL) for 2 h at room temperature under nitrogen atmosphere. Desired product could be detected by LCMS. The resulting mixture was concentrated under vacuum. The residue was purified by reversed-phase flash chromatography with the following conditions: column, C18 silica gel; mobile phase, ACN in Water (0.1% FA), 5% to 50% gradient in 25 min; detector, UV 254 nm. This resulted in 1-(tert-butoxycarbonyl)-2H,3H-pyrrolo[2,3-b]pyridin-4-ylboronic acid (55 mg). LCMS: (ESI, m/z): [M+1]⁺=265.00

Step 6: To a solution of N-(4-chloro-2-fluorophenyl)-5-(chloromethyl)-4-methylpyridin-3-amine (10 mg, 0.035 mmol) and 1-(tert-butoxycarbonyl)-2H,3H-pyrrolo[2,3-b]pyridin-4-ylboronic acid (13.89 mg, 0.053 mmol, 1.5 equiv) in dioxane (1 mL) were added K₃PO₄ (14.89 mg, 0.070 mmol), PPh₃ (1.84 mg, 0.007 mmol, 0.2 equiv) and Pd(OAc)₂ (0.79 mg, 0.004 mmol, 0.1 equiv). The resulting mixture was stirred for 16 h at 80° C. under nitrogen atmosphere. The reaction mixture was concentrated under reduced pressure and the crude product (30 mg). LCMS: (ESI, m/z): [M+1]⁺=469.30.

Step 7: To a stirred mixture of tert-butyl 4-({5-[(4-chloro-2-fluorophenyl)amino]-4-methylpyridin-3-yl}methyl)-2H,3H-pyrrolo[2,3-b]pyridine-1-carboxylate (30 mg, 0.064 mmol) in DCM (0.4 mL) was added TFA (0.1 mL) at 0° C. The resulting mixture was stirred for 2 h at room temperature. Desired product could be detected by LCMS. The reaction mixture was concentrated under vacuum. The residue was basified to pH 10 with sat. NaHCO₃ (aq.). The resulting mixture was extracted with EA (3×10 mL). The combined organic layers were washed with brine (1×30 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 70% gradient in 30 min; detector, UV 254 nm. This resulted in N-(4-chloro-2-fluorophenyl)-4-methyl-5-{1H, 2H, 3H-pyrrolo[2,3-b]pyridin-4-ylmethyl}pyridin-3-amine (10 mg). LCMS: (ESI, m/z): [M+1]⁺=369.30. ¹H NMR (400 MHz, Chloroform-d) δ 8.39 (s, 1H), 8.17 (s, 1H), 7.71 (d, J=5.5 Hz, 1H), 7.12 (dd, J=10.9, 2.3 Hz, 1H), 7.01-6.91 (m, 1H), 6.71 (t, J=8.9 Hz, 1H), 6.15 (d, J=5.5 Hz, 1H), 5.42 (s, 1H), 4.68 (s, 1H), 3.86 (s, 2H), 3.65 (t, J=8.4 Hz, 2H), 2.98 (t, J=8.4 Hz, 2H), 2.10 (s, 3H).

Step 8: To a stirred solution of N-(4-chloro-2-fluorophenyl)-4-methyl-5-{1H, 2H, 3H-pyrrolo[2,3-b]pyridin-4-ylmethyl}pyridin-3-amine (8 mg, 0.022 mmol) and pyridine (8.58 mg, 0.110 mmol, 5 equiv) in DMA (0.5 mL) was added N-methylsulfamoyl chloride (5.62 mg, 0.044 mmol) in DMA (0.2 mL) dropwise at 0° C. under air atmosphere. The resulting mixture was stirred for 2 h at room temperature. Desired product could be detected by LCMS. The reaction mixture was diluted with water (10 mL) and extracted with EA (3×10 mL). The combined organic layers were washed with brine (1×10 mL), dried over anhydrous Na₂SO₄. After filtration, the filtrate was concentrated under reduced pressure. The residue was purified by reversed-phase flash chromatography with the following conditions: Column: Aeris PEPTIDE 5 um XB-C18 Axia, 21.2 mm×250 mm, 5 μm; Mobile Phase A: Water (10 mmol/L NH₄HCO₃), Mobile Phase B: ACN; Flow rate: 60 mL/min; Gradient: 33% B to 51% B in 10 min; Wave Length: 220/254 nm; RT1 (min): 11.37. This resulted in 4-({5-[(4-chloro-2-fluorophenyl)amino]-4-methylpyridin-3-yl}methyl)-N-methyl-2H,3H-pyrrolo[2,3-b]pyridine-1-sulfonamide (5.6 mg) . . . . LCMS: (ESI, m/z): [M+1]⁺=462.10. ¹H NMR (400 MHz, Methanol-d₄) δ 8.11 (d, J=1.0 Hz, 1H), 8.05 (s, 1H), 7.99-7.93 (m, 1H), 7.23-7.15 (m, 1H), 7.06-6.98 (m, 1H), 6.71 (t, J=8.9 Hz, 1H), 6.54 (d, J=5.4 Hz, 1H), 4.08-4.00 (m, 4H), 3.09 (t, J=8.4 Hz, 2H), 2.65 (s, 3H), 2.12 (s, 3H). ¹⁹F NMR (377 MHz, Methanol-d₄) δ −128.128.

### Example 44: [(4-{[5-(4-chloro-2-fluorophenyl)-4-methylpyridin-3-yl]methyl}-3-fluoropyridin-2-yl)sulfamoyl](methyl)amine

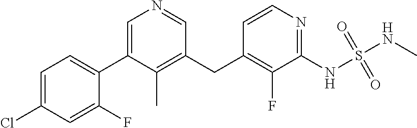 [SMILES: CNS(=O)(=O)Nc1nccc(Cc2cncc(-c3ccc(Cl)cc3F)c2C)c1F]

### Route

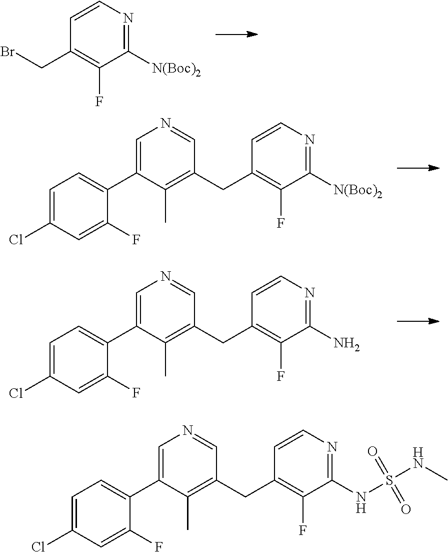

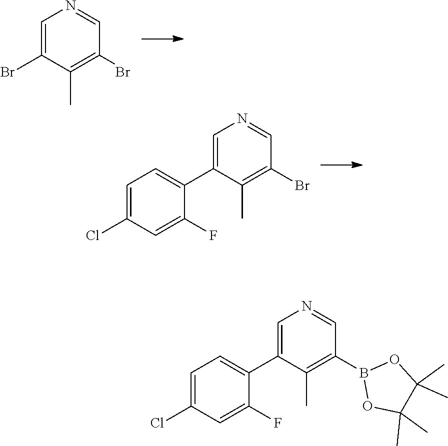

Step 1: A mixture of 3,5-dibromo-4-methylpyridine (2 g, 7.971 mmol) and 4-chloro-2-fluorophenylboronic acid (1.392 g, 7.983 mmol), Cs₂CO₃ (5.2 g, 15.960 mmol) and Pd(PPh₃)₄ (920 mg, 0.796 mmol) in dioxane (12.5 mL) and H₂O (2.5 mL) was stirred for 2 h at 80° C. under a nitrogen atmosphere. Desired product could be detected by LCMS. The resulting mixture was concentrated under reduced pressure. The residue was purified by silica gel column chromatography, eluted with PE/EA (5:1) to afford 3-bromo-5-(4-chloro-2-fluorophenyl)-4-methylpyridine (1.1 g). LCMS: (ESI, m/z): [M+1]⁺=299.95. ¹H NMR (400 MHz, Chloroform-d) δ 8.70 (s, 1H), 8.30 (s, 1H), 7.28 (d, J=2.1 Hz, 1H), 7.23 (m, 1H), 7.19 (m, 1H), 2.28 (s, 3H). ¹⁹F NMR (376 MHz, Chloroform-d) δ −111.11.

Step 2: A mixture of 3-bromo-5-(4-chloro-2-fluorophenyl)-4-methylpyridine (400 mg, 1.331 mmol), 4,4,5,5-tetramethyl-2-(tetramethyl-1,3,2-dioxaborolan-2-yl)-1,3,2-dioxaborolane (405.55 mg, 1.597 mmol, 1.2 equiv), AcOK (261.22 mg, 2.662 mmol) and Pd(dppf)Cl₂ (97.38 mg, 0.133 mmol, 0.1 equiv) in dioxane (5 mL) was stirred for 16 h at 100° C. under nitrogen atmosphere. Desired product could be detected by LCMS. The resulting mixture was concentrated under reduced pressure. This resulted in 3-(4-chloro-2-fluorophenyl)-4-methyl-5-(4,4,5,5-tetramethyl-1,3,2-dioxaborolan-2-yl)pyridine. LCMS: (ESI, m/z): [M+1]⁺=348.25

Step 3: A mixture of tert-butyl N-[4-(bromomethyl)-3-fluoropyridin-2-yl]-N-(tert-butoxycarbonyl) carbamate (200 mg, 0.494 mmol), 3-(4-chloro-2-fluorophenyl)-4-methyl-5-(4,4,5,5-tetramethyl-1,3,2-dioxaborolan-2-yl)pyridine (257.33 mg, 0.741 mmol, 1.5 equiv), K₂CO₃ (204.61 mg, 1.482 mmol, 3 equiv) and Pd(dppf)Cl₂ (36.11 mg, 0.049 mmol, 0.1 equiv) in dioxane (5 mL) and H₂O (0.5 mL) was stirred for 2 h at 80° C. under nitrogen atmosphere. Desired product could be detected by LCMS. The resulting mixture was concentrated under reduced pressure. The residue was purified by silica gel column chromatography, eluted with PE/EA (2:1) to afford tert-butyl N-(tert-butoxycarbonyl)-N-(4-{[5-(4-chloro-2-fluorophenyl)-4-methylpyridin-3-yl]methyl}-3-fluoropyridin-2-yl) carbamate (83 mg). LCMS: (ESI, m/z): [M+1]⁺=546.25. ¹H NMR (300 MHz, Chloroform-d) δ 8.40 (d, J=10.1 Hz, 2H), 8.22 (d, J=3.8 Hz, 1H), 7.27-7.16 (m, 3H), 6.95 (s, 1H), 4.14 (s, 2H), 2.09 (s, 3H), 1.41 (s, 18H). ¹⁹F NMR (282 MHz, Chloroform-d) δ −111.27, −130.87.

Step 4: To a stirred solution of tert-butyl N-(tert-butoxycarbonyl)-N-(4-{[5-(4-chloro-2-fluorophenyl)-4-methylpyridin-3-yl]methyl}-3-fluoropyridin-2-yl) carbamate (73 mg, 0.134 mmol) in DCM (0.8 mL) was added TFA (0.2 mL) dropwise at 0° C. under nitrogen atmosphere. The resulting mixture was stirred for 1 h at room temperature. Desired product could be detected by LCMS. The reaction mixture was basified to pH 10 with sat. NaHCO₃ (aq.). The resulting mixture was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 70% gradient in 30 min; detector, UV 254 nm. This resulted in 4-{[5-(4-chloro-2-fluorophenyl)-4-methylpyridin-3-yl]methyl}-3-fluoropyridin-2-amine (37 mg). LCMS: (ESI, m/z): [M+1]⁺=345.95. ¹H NMR (400 MHz, Chloroform-d) δ 8.38 (d, J=24.3 Hz, 2H), 7.72 (d, J=5.4 Hz, 1H), 7.26-7.16 (m, 3H), 6.32 (t, J=5.2 Hz, 1H), 5.11 (s, 2H), 4.05 (s, 2H), 2.09 (d, J=1.8 Hz, 3H). ¹⁹F NMR (376 MHz, Chloroform-d) δ −111.28, −144.13.

Step 5: To a stirred solution of 4-{[5-(4-chloro-2-fluorophenyl)-4-methylpyridin-3-yl]methyl}-3-fluoropyridin-2-amine (30 mg, 0.087 mmol) in DMA (1 mL) were added pyridine (34.31 mg, 0.435 mmol, 5 equiv) and N-methylsulfamoyl chloride (13.49 mg, 0.104 mmol, 1.2 equiv) in DMA (0.2 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in [(4-{[5-(4-chloro-2-fluorophenyl)-4-methylpyridin-3-yl]methyl}-3-fluoropyridin-2-yl)sulfamoyl](methyl)amine (21 mg). LCMS: (ESI, m/z): [M+1]⁺=439.05. ¹H NMR (400 MHz, DMSO-d₆) δ 10.37 (s, 1H), 8.41 (s, 1H), 8.31 (s, 1H), 8.00 (d, J=4.9 Hz, 1H), 7.63-7.57 (m, 1H), 7.48-7.40 (m, 2H), 7.00 (s, 1H), 6.77 (s, 1H), 4.15 (s, 2H), 3.36 (s, 3H), 2.04 (d, J=1.5 Hz, 3H). ¹⁹F NMR (377 MHz, DMSO-d₆) δ −111.87, −138.03.

### Example 45: 4-[[5-(4-chloro-2-fluoro-anilino)-4-methyl-3-pyridyl]methyl]-3-methoxy-N-(methylsulfamoyl)pyridin-2-amine

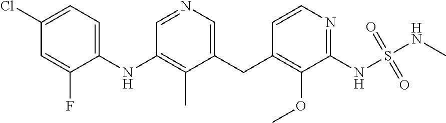 [SMILES: CNS(=O)(=O)Nc1nccc(Cc2cncc(Nc3ccc(Cl)cc3F)c2C)c1OC]

### Route

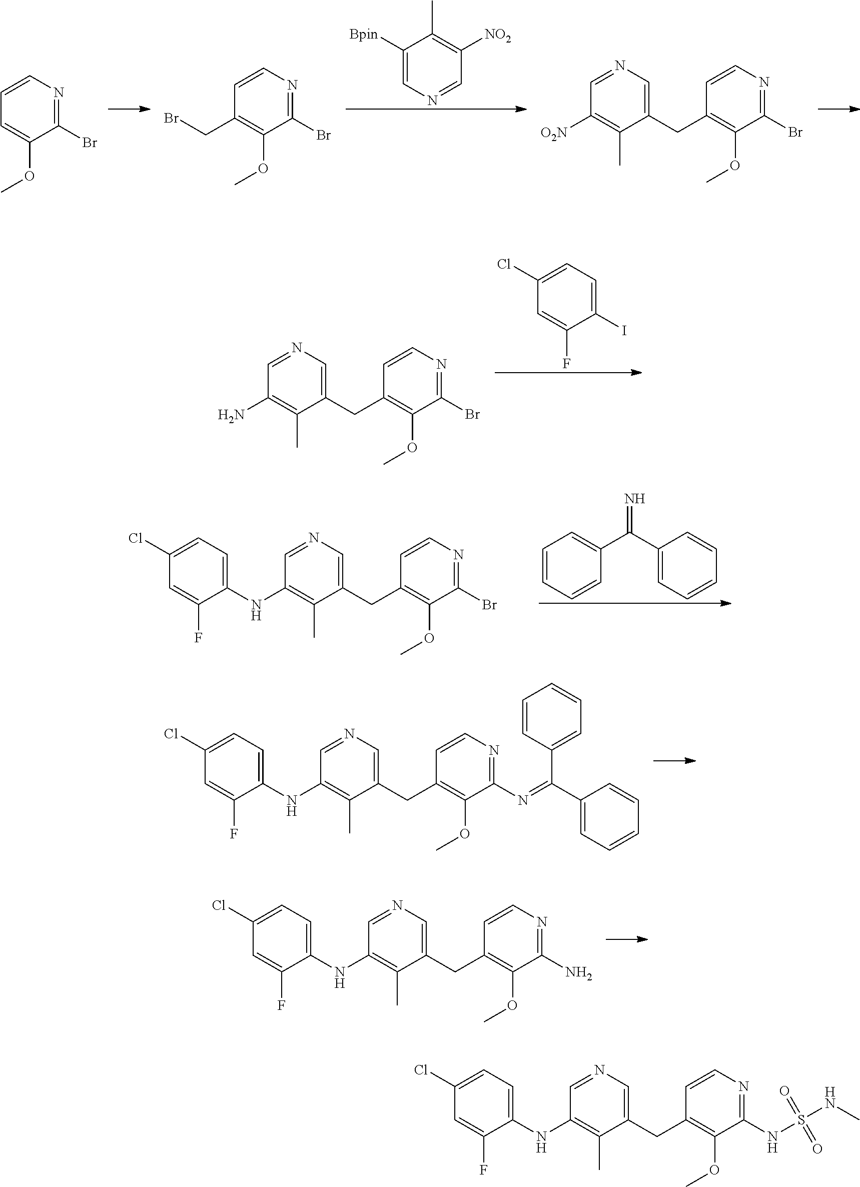

Step 1: LDA (2 M, 15.96 mL) was added dropwise to THF (25 mL) under N₂ at −78° C. A solution of 2-bromo-3-methoxy-pyridine (5 g, 26.59 mmol) in THF (2 mL) was added slowly to the above solution at −78° C. The reaction mixture was kept at −78° C. and stirred for 1 h. N,N-dimethylformamide (2.33 g, 31.91 mmol, 2.46 mL) in THF (2 mL) was added to the solution slowly. The mixture was stirred at −78° C. for 1 h. NaBH₄ (1.26 g, 33.30 mmol) was added to the solution at 0° C. The mixture was stirred for 2 h under N₂. The reaction mixture was quenched with saturated NH₄Cl solution (100 mL). The aqueous layer was extracted with EtOAc (100 mL×3). The combined organic layers were washed with brine (100 mL), dried over anhydrous Na₂SO₄, filtered and concentrated. The solid was purified by flash chromatography on silica gel (ethyl acetate in petroleum ether=0-50%) to afford (2-bromo-3-methoxy-4-pyridyl) methanol (3.5 g, 16.05 mmol). ¹H NMR (400 MHz, CDCl₃) δ=8.15 (d, J=4.8 Hz, 1H), 7.40 (d, J=4.8 Hz, 1H), 4.81 (d, J=4.8 Hz, 2H), 3.90 (s, 3H), 2.35 (br s, 1H).

Step 2: To a solution of (2-bromo-3-methoxy-4-pyridyl) methanol (2 g, 9.17 mmol) in DCM (20 mL) was added tribromophosphane (3.23 g, 11.92 mmol, 1.12 mL) dropwise at 0° C. under N₂. The mixture was stirred at 25° C. for 1 hr. The mixture was added dropwise into H₂O (50 mL) at 0° C. The mixture was adjusted to pH=7 with saturated NaHCO₃ solution slowly at 0° C. The aqueous layer was extracted with DCM (50 mL×2). The combined organic layers were washed with brine (50 mL), dried over anhydrous Na₂SO₄, filtered and concentrated to give 2-bromo-4-(bromomethyl)-3-methoxy-pyridine (2.4 g, 8.54 mmol) 1H NMR (400 MHz, CDCl₃) δ=8.12 (d, J=4.8 Hz, 1H), 7.274 (d, J=4.8 Hz, 1H), 4.46 (s, 2H), 4.00 (s, 3H).

Step 3: To a solution of 2-bromo-4-(bromomethyl)-3-methoxy-pyridine (2.4 g, 8.54 mmol) in toluene (16 mL) and EtOH (4 mL) were added 4-methyl-3-nitro-5-(4,4,5,5-tetramethyl-1,3,2-dioxaborolan-2-yl)pyridine (1.88 g, 7.12 mmol), Pd(PPh₃)₄ (411.31 mg, 355.94 μmol) and Na₂CO₃ (1.51 g, 14.24 mmol) in H₂O (2 mL). The mixture was stirred at 80° C. for 2 hr. The reaction mixture was poured into water (20 mL) and extracted with DCM (50 mL×3). The combined organic layers were washed with brine (100 mL), dried over anhydrous Na₂SO₄, filtered and concentrated. The mixture was purified by flash chromatography on silica gel (ethyl acetate in petroleum ether=0-25%) to afford 2-bromo-3-methoxy-4-[(4-methyl-5-nitro-3-pyridyl)methyl]pyridine (1.24 g, 3.67 mmol). ¹H NMR (400 MHz, CDCl₃) δ=8.97 (s, 1H), 8.65 (d, J=4.8 Hz, 1H), 7.77 (d, J=4.8 Hz, 1H), 4.16 (s, 2H), 3.91 (s, 3H), 2.39 (s, 3H).

Step 4: To a solution of 2-bromo-3-methoxy-4-[(4-methyl-5-nitro-3-pyridyl)methyl]pyridine (1.2 g, 3.55 mmol) in EtOH (5 mL) and EtOAc (5 mL) was added SnCl₂·2H₂O (4.00 g, 17.74 mmol). The mixture was stirred at 90° C. for 2 hr. After cooling to room temperature, the mixture was concentrated under reduced pressure to remove EtOH. Sat. NaHCO₃ solution was added to the reaction mixture dropwise until pH=10. The mixture was extracted with EtOAc (20 mL×3). The combined organic layers were washed with brine (20 mL), dried over anhydrous Na₂SO₄, filtered and concentrated. The crude was purified by flash chromatography on silica gel (ethyl acetate in petroleum ether=0-25%) to afford 5-[(2-bromo-3-methoxy-4-pyridyl)methyl]-4-methyl-pyridin-3-amine (1.04 g, 3.37 mmol). 1H NMR (400 MHz, CDCl₃) δ=8.01 (s, 1H), 7.98 (d, J=4.8 Hz, 1H), 7.84 (s, 1H), 6.73 (d, J=4.8 Hz, 1H), 4.08 (s, 2H), 3.90 (s, 3H), 3.71 (br s, 2H), 1.94 (s, 3H).

Step 5: To a solution of 5-[(2-bromo-3-methoxy-4-pyridyl)methyl]-4-methyl-pyridin-3-amine (950 mg, 3.08 mmol) in dioxane (10 mL) were added 4-chloro-2-fluoro-1-iodo-benzene (1.58 g, 6.17 mmol), Pd(OAc)₂ (69.21 mg, 308.27 μmol,), Xantphos (356.74 mg, 616.54 μmol) and Cs₂CO₃ (2.01 g, 6.17 mmol). The mixture was stirred at 70° C. for 3 hr. The reaction mixture was poured into water (50 mL) and extracted with EtOAc (30 mL×3). The combined organic layers were washed with brine (50 mL), dried over anhydrous Na₂SO₄, filtered and concentrated. The mixture was purified by flash chromatography on silica gel (ethyl acetate in petroleum ether=0-25%) to afford 5-[(2-bromo-3-methoxy-4-pyridyl)methyl]-N-(4-chloro-2-fluoro-phenyl)-4-methyl-pyridin-3-amine (1 g, 2.29 mmol). ¹H NMR (400 MHz, CDCl₃) δ=8.37 (s, 1H), 8.16 (s, 1H), 8.03 (d, J=4.8 Hz, 1H), 7.12 (dd, J=10.8 Hz, 2.4 Hz, 1H), 6.99 (d, J=8.4 Hz, 1H), 6.82-6.77 (m, 2H), 5.52 (br s, 1H), 4.10 (s, 2H), 3.91 (s, 3H), 2.11 (s, 3H).

Step 6: To a solution of 5-[(2-bromo-3-methoxy-4-pyridyl)methyl]-N-(4-chloro-2-fluoro-phenyl)-4-methyl-pyridin-3-amine (500 mg, 1.14 mmol) in toluene (5 mL) were added diphenylmethanimine (249.00 mg, 1.37 mmol, 230.56 μL), Pd₂(dba)₃ (209.69 mg, 228.99 umol), Xantphos (264.99 mg, 457.98 μmol) and NaOtBu (264.08 mg, 2.75 mmol). The mixture was stirred at 110° C. for 4 hr. The reaction mixture was quenched with water (50 mL). The mixture was extracted with EtOAc (50 mL×3). The combined organic layers were washed with brine (50 mL), dried over anhydrous Na₂SO₄, filtered and concentrated. The crude was purified by flash chromatography on silica gel (ethyl acetate in petroleum ether=0-100%) to afford 5-[[2-(benzhydrylideneamino)-3-methoxy-4-pyridyl]methyl]-N-(4-chloro-2-fluoro-phenyl)-4-methyl-pyridin-3-amine (200 mg, 372.42 μmol). ¹H NMR (400 MHz, CDCl₃) δ=8.36 (s, 1H), 8.02 (s, 1H), 7.91 (d, J=4.8 Hz, 1H), 7.80 (s, 2H), 7.55-7.35 (m, 4H), 7.25-7.15 (m, 5H), 6.95 (d, J=8 Hz, 1H), 6.65 (t, J=8.8 Hz, 1H), 6.40-6.35 (m, 1H), 5.42 (br s, 1H), 3.91 (s, 2H), 3.78 (s, 3H), 1.80 (s, 3H). ¹⁹F NMR (376.5 MHz, CDCl₃) δ=−130.571.

Step 7: To a solution of 5-[[2-(benzhydrylideneamino)-3-methoxy-4-pyridyl]methyl]-N-(4-chloro-2-fluoro-phenyl)-4-methyl-pyridin-3-amine (200 mg, 372.42 μmol) in MeOH (0.5 mL) was added HCl/MeOH (4 M, 2 mL). The mixture was stirred at 25° C. for 5 hr. The mixture was added dropwise into H₂O (20 mL). The mixture was adjusted to pH=7 with saturated NaHCO₃ solution slowly. The solution was extracted with EtOAc (50 mL×3). The combined organic layers were washed with brine (20 mL), dried over anhydrous Na₂SO₄, filtered and concentrated. The mixture was purified by flash chromatography on silica gel (ethyl acetate in petroleum ether=0-25%) to afford 4-[[5-(4-chloro-2-fluoro-anilino)-4-methyl-3-pyridyl]methyl]-3-methoxy-pyridin-2-amine (100 mg, 268.22 μmol). ¹H NMR (400 MHz, CDCl₃) δ=8.37 (s, 1H), 8.18 (s, 1H), 7.69 (d, J=5.2 Hz, 1H), 7.10 (dd, J=2.0 Hz, J=10.8 Hz, 1H), 6.95 (d, J=8.8 Hz, 1H), 6.69 (t, J=8.8 Hz, 1H), 6.19 (d, J=5.2 Hz, 1H), 5.50 (s, 1H), 4.75 (br s, 2H), 3.99 (s, 2H), 3.77 (s, 3H), 2.09 (s, 3H). ¹⁹F NMR (376.5 MHz, CDCl₃) δ=−130.614.

Step 8: To a solution of 4-[[5-(4-chloro-2-fluoro-anilino)-4-methyl-3-pyridyl]methyl]-3-methoxy-pyridin-2-amine (20 mg, 53.64 μmol) in MeCN (1 mL) were added N-methylsulfamoyl chloride (34.75 mg, 268.22 μmol, 5 eq) and Py (42.43 mg, 536.45 μmol, 43.30 μL). The mixture was stirred at 25° C. for 1.5 hr. The reaction solution was concentrated. The solid was blended with another batch prepared from 14 mg 4-[[5-(4-chloro-2-fluoro-anilino)-4-methyl-3-pyridyl]methyl]-3-methoxy-pyridin-2-amine. The crude was purified by perp-HPLC (column: Boston Green ODS 150*30 mm*5 um; mobile phase: [water(FA)−ACN]; B %: 15%-45%, 6 min) to give 4-[[5-(4-chloro-2-fluoro-anilino)-4-methyl-3-pyridyl]methyl]-3-methoxy-N-(methylsulfamoyl)pyridin-2-amine (6 mg, 12.88 μmol). 1H NMR (400 MHz, CDCl₃) δ=8.41 (s, 1H), 8.18 (s, 1H), 7.97-7.83 (m, 1H), 7.54-7.33 (m, 1H), 7.14 (d, J=10.8 Hz, 1H), 6.98 (d, J=8.8 Hz, 1H), 6.75 (t, J=8.8 Hz, 1H), 6.50 (d, J=4.8 Hz, 1H), 5.57 (s, 1H), 5.42 (s, 1H), 4.05 (s, 2H), 3.85 (s, 3H), 2.75 (s, 3H), 2.08 (s, 3H). ¹⁹F NMR (376.5 MHz, CDCl₃) δ=−130.176. LCMS Rₜ=0.674 min in 1.5 min chromatography, 5-95AB, ESI calcd. for C₂₀H₂₂ClFN₅O₃S [M+H]⁺466.1, found 466.1.

### Example 46: 5-[[5-[(5-chloro-3-fluoro-2-pyridyl)oxy]-4-methyl-3-pyridyl]methyl]-N-(methylsulfamoyl)pyridin-2-amine

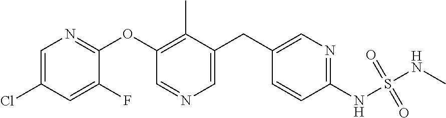 [SMILES: CNS(=O)(=O)Nc1ccc(Cc2cncc(Oc3ncc(Cl)cc3F)c2C)cn1]

### Route

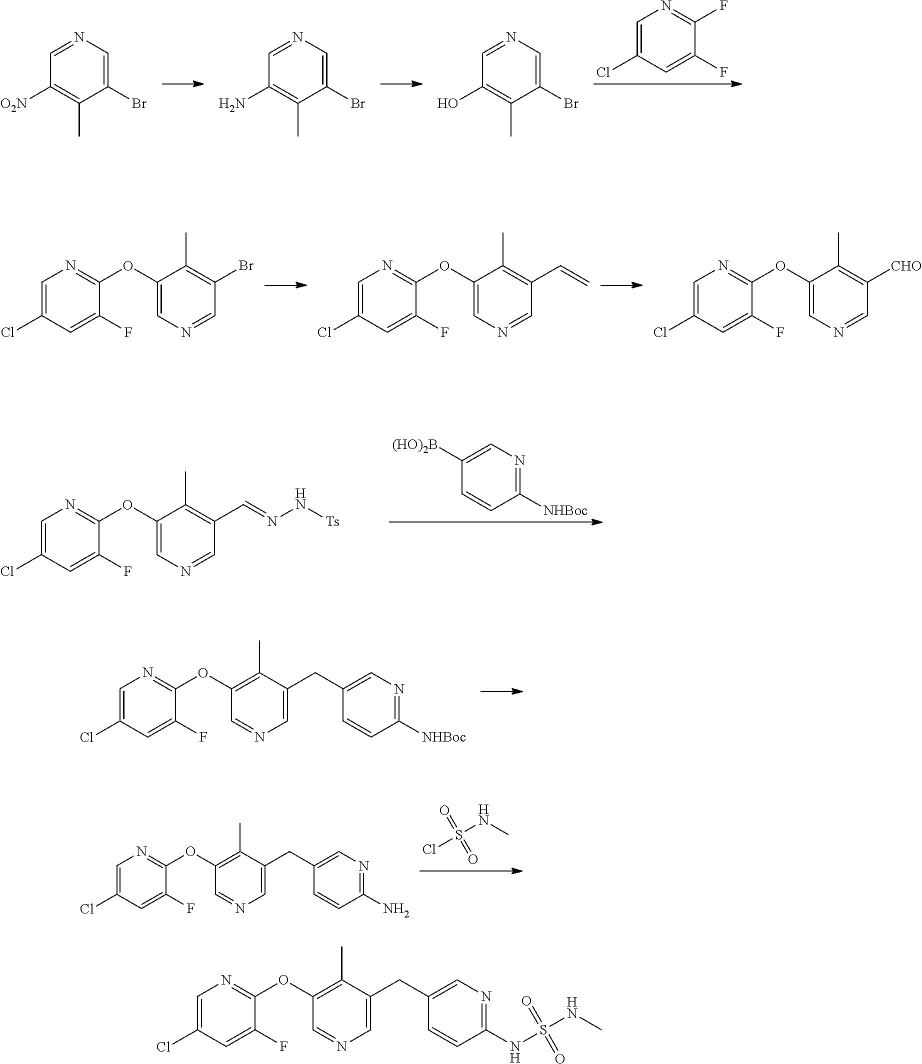

Step 1: To a solution of 3-bromo-4-methyl-5-nitro-pyridine (50 g, 230.39 mmol) in EtOH (1250 mL) and H₂O (25 0 mL) were added Fe (128.66 g, 2.30 mol) and NH₄Cl (36.97 g, 691.17 mmol). The mixture was stirred at 80° C. for 12 h. After cooling to room temperature, water (200 mL) was added to the mixture and the aqueous layer was extracted with EtOAc (100 mL×3). The combined organic phase was washed with brine (100 mL), dried over anhydrous Na₂SO₄, filtered and concentrated under reduced pressure. 5-bromo-4-methyl-pyridin-3-amine (34 g, 181.78 mmol). ¹H NMR (400 MHz, CDCl₃) δ=8.11 (s, 1H), 7.92 (s, 1H), 3.53 (br s, 2H), 2.26 (s, 3H)

Step 2: To a solution of 5-bromo-4-methyl-pyridin-3-amine (10 g, 53.47 mmol) in H₂SO₄ (50 mL) was added NaNO₂ (4.06 g, 58.81 mmol) in H₂O (25 mL) at 0° C. for 0.25 h. A solution of Cu(NO₃)₂ (258.35 g, 1.07 mol) in H₂O (400 mL) was added followed by Cu₂O (8.80 g, 61.49 mmol, 6.28 mL). The mixture was stirred at 25° C. for 11.75 h. NaOH solution (1.0 M) was added to the reaction mixture until pH=7. The reaction mixture was poured into water (20 mL) and extracted with EtOAc (100 mL×3). The combined organic layers were washed with brine (100 mL), dried over anhydrous Na₂SO₄, filtered and concentrated. The solid was purified by flash chromatography on silica gel (ethyl acetate in petroleum ether=0-30%) to give 5-bromo-4-methyl-pyridin-3-ol (6.3 g, 33.51 mmol). ¹H NMR (400 MHz, CDCl₃) δ=8.17 (s, 1H), 8.06 (s, 1H), 2.38 (s, 3H). LCMS Rₜ=0.463 min in 1.5 min chromatography, 5-95AB, ESI calcd. for C₆H₇BrNO [M+H]⁺190.0, found 189.9.

Step 3: To a solution of 5-bromo-4-methyl-pyridin-3-ol (2 g, 10.64 mmol) in DMAC (20 mL) were added 5-chloro-2,3-difluoro-pyridine (3.18 g, 21.27 mmol), CsF (2.42 g, 15.96 mmol, 588.28 μL) and TEA (3.23 g, 31.91 mmol, 4.44 mL). The mixture was stirred at 80° C. for 5 h. H₂O (30 mL) was added to the mixture. The aqueous phase was extracted with EtOAc (20 mL×3) and the combined organic phase was washed with brine (20 mL×3), dried over anhydrous Na₂SO₄, filtered and concentrated. The crude was purified by flash chromatography on silica gel (ethyl acetate in petroleum ether=0-10%) to give 2-[(5-bromo-4-methyl-3-pyridyl)oxy]-5-chloro-3-fluoro-pyridine (990 mg, 3.12 mmol). ¹H NMR (400 MHz, CDCl₃) δ=8.59 (s, 1H), 8.30 (s, 1H), 7.83 (d, J=2.0 Hz, 1H), 7.56 (dd, J=2.0, 8.8 Hz, 1H), 2.29 (s, 3H). ¹⁹F NMR (376.5 MHz, CDCl₃) δ=−134.165. LCMS Rₜ=0.946 min in 1.5 min chromatography, 5-95AB, ESI calcd. for C₁₁H₈BrClFN₂O [M+H]⁺318.9, found 318.7.

Step 4: To a solution of 2-[(5-bromo-4-methyl-3-pyridyl)oxy]-5-chloro-3-fluoro-pyridine (400 mg, 1.26 mmol) and 4,4,5,5-tetramethyl-2-vinyl-1,3,2-dioxaborolane (232.81 mg, 1.51 mmol, 256.40 μL) in dioxane (4 mL) and H₂O (0.4 mL) was added K₂CO₃ (522.29 mg, 3.78 mmol). The mixture was degassed and purged with N₂ for 3 times, then Pd(dppf)Cl₂ (92.17 mg, 125.97 μmol) was added to the mixture, degassed and purged with N₂ for 3 times. The mixture was stirred at 80° C. for 3 h. The mixture was blended with two batches (prepared from 400 mg and 50 mg 2-[(5-bromo-4-methyl-3-pyridyl)oxy]-5-chloro-3-fluoro-pyridine). Water (20 mL) was added and the mixture was extracted with EtOAc (30 mL×2) and the organic layer was dried over anhydrous Na₂SO₄, filtered and concentrated. The crude was purified by flash chromatography on silica gel (ethyl acetate in petroleum ether=0-10%) to give 3-[(5-chloro-3-fluoro-2-pyridyl)oxy]-4-methyl-5-vinyl-pyridine (500 mg, 1.89 mmol). ¹H NMR (400 MHz, CDCl₃) δ=8.55 (s, 1H), 8.28 (s, 1H), 7.83 (d, J=2.0 Hz, 1H), 7.54 (dd, J=2.4, 9.2 Hz, 1H), 6.85 (dd, J=10.8, 17.6 Hz, 1H), 5.76 (dd, J=1.2, 17.6 Hz, 1H), 5.48 (dd, J=1.2, 10.8 Hz, 1H), 2.19 (s, 3H). ¹⁹F NMR (376.5 MHz, CDCl₃) δ=−134.356. LCMS Rₜ=0.791 min in 1.5 min chromatography, 5-95AB, ESI calcd. for C₁₃H₁₁ClFN₂O [M+H]⁺265.1, found 264.9.

Step 5: To a solution of 3-[(5-chloro-3-fluoro-2-pyridyl)oxy]-4-methyl-5-vinyl-pyridine (450 mg, 1.70 mmol) in THF (13.5 mL) and H₂O (2.7 mL) were added K₂OsO₄·2H₂O (62.64 mg, 170.02 μmol) and NaIO₄ (1.45 g, 6.80 mmol, 376.84 μL). The mixture was stirred at 25° C. for 1 h. The mixture was blended with another batch prepared from 50 mg 3-[(5-chloro-3-fluoro-2-pyridyl)oxy]-4-methyl-5-vinyl-pyridine. The reaction was diluted with water (20 mL) and extracted with EtOAc (20 mL×3). The combined organic phase was dried over anhydrous Na₂SO₄, filtered and concentrated under reduced pressure. The crude was purified by flash chromatography on silica gel (ethyl acetate in petroleum ether=0-20%) to give 5-[(5-chloro-3-fluoro-2-pyridyl)oxy]-4-methyl-pyridine-3-carbaldehyde (400 mg, 1.50 mmol). 1H NMR (400 MHz, CDCl₃) δ=10.34 (s, 1H), 8.87 (s, 1H), 8.57 (s, 1H), 7.81 (d, J=2.4 Hz, 1H), 7.58 (dd, J=2.4, 9.2 Hz, 1H), 2.53 (s, 3H). ¹⁹F NMR (376.5 MHz, CDCl₃) δ=−134.181. LCMS Rₜ=0.811 min in 1.5 min chromatography, 5-95AB, ESI calcd. for C₁₂H₉ClFN₂O₂ [M+H]⁺267.0, found 266.9.

Step 6: To a solution of 5-[(5-chloro-3-fluoro-2-pyridyl)oxy]-4-methyl-pyridine-3-carbaldehyde (300 mg, 1.13 mmol) in MeOH (4 mL) was added 4-methylbenzenesulfonohydrazide (209.52 mg, 1.13 mmol). The mixture was stirred at 60° C. for 2 h. The mixture was blended with another batch prepared form 100 mg 5-[(5-chloro-3-fluoro-2-pyridyl)oxy]-4-methyl-pyridine-3-carbaldehyde. The mixture was concentrated directly. N-[(E)-[5-[(5-chloro-3-fluoro-2-pyridyl)oxy]-4-methyl-3-pyridyl]methyleneamino]-4-methyl-benzenesulfonamide (600 mg, 1.38 mmol). ¹H NMR (400 MHz, CDCl₃) δ=8.66 (s, 1H), 8.35 (s, 1H), 8.01 (s, 1H), 7.86 (d, J=8.4 Hz, 2H), 7.78 (d, J=2.4 Hz, 1H), 7.55 (dd, J=2.4, 9.2 Hz, 1H), 7.32 (d, J=8.4 Hz, 2H), 2.42 (s, 3H), 2.25 (s, 3H). ¹⁹F NMR (376.5 MHz, CDCl₃) δ=−134.211. LCMS Rₜ=0.867 min in 1.5 min chromatography, 5-95AB, ESI calcd. for C₁₉H₁₇ClFN₄O₃S [M+H]⁺435.1, found 434.9.

Step 7: To a solution of N-[(E)-[5-[(5-chloro-3-fluoro-2-pyridyl)oxy]-4-methyl-3-pyridyl]methyleneamino]-4-methyl-benzenesulfonamide (240 mg, 551.89 μmol) and [6-(tert-butoxycarbonylamino)-3-pyridyl]boronic acid (262.75 mg, 1.10 mmol) in dioxane (3 mL) was added K₂CO₃ (228.82 mg, 1.66 mmol). The mixture was stirred at 110° C. for 2 h. The mixture was blended with another batch prepared form 30 mg N-[(E)-[5-[(5-chloro-3-fluoro-2-pyridyl)oxy]-4-methyl-3-pyridyl]methyleneamino]-4-methyl-benzenesulfonamide. H₂O (30 mL) was added to the mixture. The aqueous phase was extracted with EtOAc (30 mL×3). The combined organic phase was washed with brine (20 mL×3), dried over anhydrous Na₂SO₄, filtered and concentrated. The crude was purified by flash chromatography on silica gel (ethyl acetate in petroleum ether=0-60%) to give tert-butyl N-[5-[[5-[(5-chloro-3-fluoro-2-pyridyl)oxy]-4-methyl-3-pyridyl]methyl]-2-pyridyl]carbamate (140 mg, 314.69 μmol). ¹H NMR (400 MHz, CDCl₃) δ=8.32 (s, 1H), 8.28 (s, 1H), 8.05 (d, J=2.0 Hz, 1H), 7.89 (d, J=8.4 Hz, 1H), 7.82 (d, J=2.0 Hz, 1H), 7.56-7.52 (m, 2H), 7.43 (dd, J=2.0, 8.8 Hz, 1H), 3.99 (s, 2H), 2.07 (s, 3H), 1.52 (s, 9H). ¹⁹F NMR (376.5 MHz, CDCl₃) δ=−134.319. LCMS Rₜ=0.840 min in 1.5 min chromatography, 5-95AB, ESI calcd. for C₂₂H₂₃ClFN₄O₃ [M+H]⁺445.1, found 445.1.

Step 8: To a solution of tert-butyl N-[5-[[5-[(5-chloro-3-fluoro-2-pyridyl)oxy]-4-methyl-3-pyridyl]methyl]-2-pyridyl]carbamate (120 mg, 269.73 μmol) in MeOH (2 mL) was added HCl/MeOH (4 M, 2.70 mL). The mixture was stirred at 25° C. for 2 h. The mixture was blended with another batch prepared from 20 mg N-[5-[[5-[(5-chloro-3-fluoro-2-pyridyl)oxy]-4-methyl-3-pyridyl]methyl]-2-pyridyl]carbamate. Sat. NaHCO₃ solution was added to the mixture until pH=7. The reaction mixture was extracted with EtOAc (30 mL×3) and the combined organic phase was washed with brine (50 mL), dried over anhydrous Na₂SO₄, filtered and concentrated. The crude was purified by flash chromatography on silica gel (dichloromethane in methanol=0-10%) to give 5-[[5-[(5-chloro-3-fluoro-2-pyridyl)oxy]-4-methyl-3-pyridyl]methyl]pyridin-2-amine (100 mg, 290.05 μmol). ¹H NMR (400 MHz, CDCl₃) δ=8.28 (d, J=6.4 Hz, 2H), 7.88 (s, 1H), 7.82 (d, J=2.0 Hz, 1H), 7.53 (dd, J=2.0, 9.2 Hz, 1H), 7.22 (dd, J=2.4, 8.8 Hz, 1H), 6.49 (d, J=8.8 Hz, 1H), 4.63 (br s, 2H), 3.90 (s, 2H), 2.08 (s, 3H). ¹⁹F NMR (376.5 MHz, CDCl₃) δ=−134.368. LCMS Rₜ=0.688 min in 1.5 min chromatography, 5-95AB, ESI calcd. for C₁₇H₁₅ClFN₄O [M+H]⁺345.1, found 345.1.

Step 9: To a solution of 5-[[5-[(5-chloro-3-fluoro-2-pyridyl)oxy]-4-methyl-3-pyridyl]methyl]pyridin-2-amine (80 mg, 232.04 μmol) in MeCN (1 mL) were added Py (220.25 mg, 2.78 mmol, 224.75 μL) and N-methylsulfamoyl chloride (60.13 mg, 464.08 μmol). The mixture was stirred at 25° C. for 2 h. The mixture was blended with another batch prepared from 20 mg 5-[[5-[(5-chloro-3-fluoro-2-pyridyl)oxy]-4-methyl-3-pyridyl]methyl]pyridin-2-amine. The mixture was concentrated directly and purified by prep-HPLC (column: Boston Green ODS 150*30 mm*5 um; mobile phase: [water(FA)−ACN]; B %: 38%-68%, 6 min) to afford 5-[[5-[(5-chloro-3-fluoro-2-pyridyl)oxy]-4-methyl-3-pyridyl]methyl]-N-(methylsulfamoyl)pyridin-2-amine (23 mg, 52.53 umol). ¹H NMR (400 MHz, CDCl₃) δ=8.42-8.22 (m, 2H), 8.13 (d, J=1.2 Hz, 1H), 7.83 (d, J=2.4 Hz, 1H), 7.55 (dd, J=2.4, 9.2 Hz, 1H), 7.43 (dd, J=2.4, 8.8 Hz, 1H), 7.00 (d, J=8.8 Hz, 1H), 5.03 (br s, 1H), 4.01 (s, 2H), 2.73 (s, 3H), 2.13 (s, 3H). ¹⁹F NMR (376.5 MHz, CDCl₃) δ=−134.174. LCMS Rₜ=0.769 min in 1.5 min chromatography, 5-95AB, ESI calcd. for C₁₈H₁₈ClFN₅O₃S [M+H]⁺438.1, found 438.1. HPLC Rₜ=2.908 min in 8 min chromatography, 220 nm, purity 98.116%.

### Example 47: 4-[[5-(4-cyclopropyl-2-fluoro-anilino)-4-methyl-3-pyridyl]methyl]-3-fluoro-N-(methylsulfamoyl)pyridin-2-amine

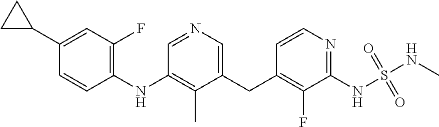 [SMILES: CNS(=O)(=O)Nc1nccc(Cc2cncc(Nc3ccc(C4CC4)cc3F)c2C)c1F]

### Route

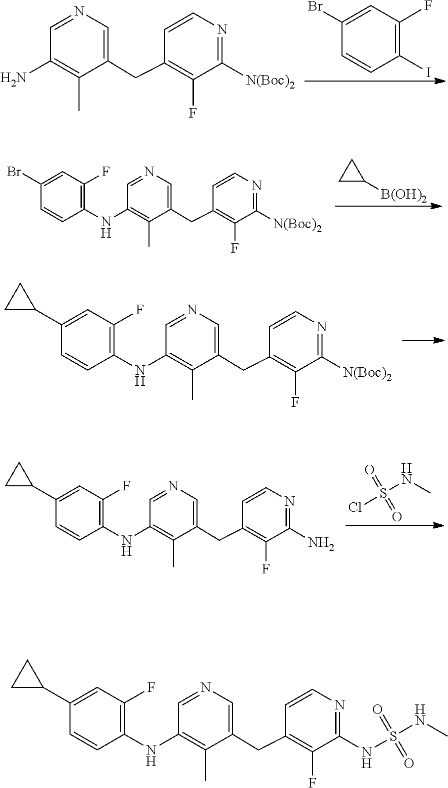

Step 1: 4-bromo-2-fluoro-1-iodo-benzene (469.62 mg, 1.56 mmol) and Pd₂(dba)₃ (47.64 mg, 52.02 μmol) are added under nitrogen to a solution of tert-butyl N-[4-[(5-amino-4-methyl-3-pyridyl)methyl]-3-fluoro-2-pyridyl]-N-tert-butoxycarbonyl-carbamate (450 mg, 1.04 mmol, example 6) in dioxane (10 mL). The medium is degassed for 5 minutes under N₂ before adding Cs₂CO₃ (474.62 mg, 1.46 mmol) and Xantphos (60.21 mg, 104.05 μmol). The reaction mixture is stirred at 80° C. for 2 hours. The mixture was concentrated. The mixture was purified by flash chromatography on silica gel (EtOAc in petroleum ether=0-70%) to afford tert-butyl N-[4-[[5-(4-bromo-2-fluoro-anilino)-4-methyl-3-pyridyl]methyl]-3-fluoro-2-pyridyl]-N-tert-butoxycarbonyl-carbamate (350 mg, 578.06 μmol). NMR (400 MHz, CDCl₃) δ=8.38 (s, 1H), 8.22-8.11 (m, 2H), 7.31-7.26 (m, 1H), 7.12 (d, J=8.4 Hz, 1H), 6.90 (t, J=5.2 Hz, 1H), 6.80-6.58 (m, 1H), 4.10 (s, 2H), 2.12 (s, 3H), 1.42 (s, 18H).

Step 2: To a solution of tert-butyl N-[4-[[5-(4-bromo-2-fluoro-anilino)-4-methyl-3-pyridyl]methyl]-3-fluoro-2-pyridyl]-N-tert-butoxycarbonyl-carbamate (160 mg, 264.26 μmol) in dioxane (3 mL) was added K3PO4 (168.28 mg, 792.77 μmol), Pd(dppf)Cl₂ (19.34 mg, 26.43 μmol) and CsF (20.07 mg, 132.13 umol, 4.87 μL) and cyclopropylboronic acid (226.99 mg, 2.64 mmol). The mixture was stirred at 75° C. for 3 h. The mixture was concentrated. The mixture was purified by flash chromatography on silica gel (MeOH in DCM=0-4%) to afford tert-butyl N-tert-butoxycarbonyl-N-[4-[[5-(4-cyclopropyl-2-fluoro-anilino)-4-methyl-3-pyridyl]methyl]-3-fluoro-2-pyridyl]carbamate (149.7 mg, 264.19 μmol). LCMS Rt=5.035 min in 7 min chromatography, 10-80 CD, ESI calcd. for C₃₁H₃₇F₂N₄O₄ [M+H]⁺567.3, found 567.3.

Step 3: To a solution of tert-butyl N-tert-butoxycarbonyl-N-[4-[[5-(4-cyclopropyl-2-fluoro-anilino)-4-methyl-3-pyridyl]methyl]-3-fluoro-2-pyridyl]carbamate (149.7 mg, 264.19 μmol) in MeOH (2 mL) was added HCl/MeOH (4 M, 7.25 mL). The mixtur was stirred at 20° C. for 3 h. The mixture was concentrated. The mixture was added to NH₃/MeOH (3 mL) to adjust to pH=7. The mixture was concentrated. The mixture was purified by flash chromatography on silica gel (EtOAc in petroleum ether=0-100%) to afford 4-[[5-(4-cyclopropyl-2-fluoro-anilino)-4-methyl-3-pyridyl]methyl]-3-fluoro-pyridin-2-amine (70 mg, 191.04 μmol). nLCMS Rt=1.47 min in 3 min chromatography, 5-95CD, ESI calcd. for C₂₁H₂₁F₂N₄ [M+H]⁺367.3, found 367.2.

Step 4: To a solution of 4-[[5-(4-cyclopropyl-2-fluoro-anilino)-4-methyl-3-pyridyl]methyl]-3-fluoro-pyridin-2-amine (30 mg, 81.88 μmol) in MeCN (1 mL) and was added Py (64.76 mg, 818.76 μmol, 66.09 μL) and methylsulfamoyl chloride (53.04 mg, 409.38 μmol). The mixture was stirred at 25° C. for 24 h. The mixture was concentrated. The crude was purified by Prep-HPLC (column: Phenomenex C18 75×30 mm×3 um; mobile phase: [water(NH₃H₂O+NH₄HCO₃)−ACN]; B %: 23%-53%, 8 min) to give 4-[[5-(4-cyclopropyl-2-fluoro-anilino)-4-methyl-3-pyridyl]methyl]-3-fluoro-N-(methylsulfamoyl)pyridin-2-amine (6.6 mg, 14.36 μmol). ¹H NMR (400 MHz, CDCl₃) δ=8.34 (s, 1H), 8.09 (s, 1H), 7.95 (d, J=5.2 Hz, 1H), 6.91-6.76 (m, 3H), 6.59 (t, J=5.2 Hz, 1H), 5.48 (br s, 1H), 5.32 (br s, 1H), 4.04 (s, 2H), 2.77 (s, 3H), 2.14 (s, 3H), 0.99-0.91 (m, 2H), 0.68-0.61 (m, 2H). ¹⁹F NMR (376.5 MHz, CDCl₃) δ=−131.253, −142.7631 ppm. LCMS Rt=0.766 min in 1.5 min chromatography, 5-95AB, ESI calcd. for C₂₂H₂₄F₂N₅O₂S [M+H]⁺460.2, found 460.1.

### Example 48: 5-[[5-[(5-chloro-3-fluoro-2-pyridyl)oxy]-4-methyl-3-pyridyl]methyl]-N-(methylsulfamoyl)pyridin-2-amine

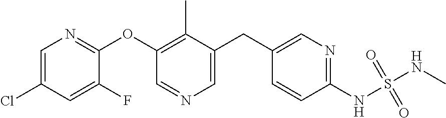 [SMILES: CNS(=O)(=O)Nc1ccc(Cc2cncc(Oc3ncc(Cl)cc3F)c2C)cn1]

### Route

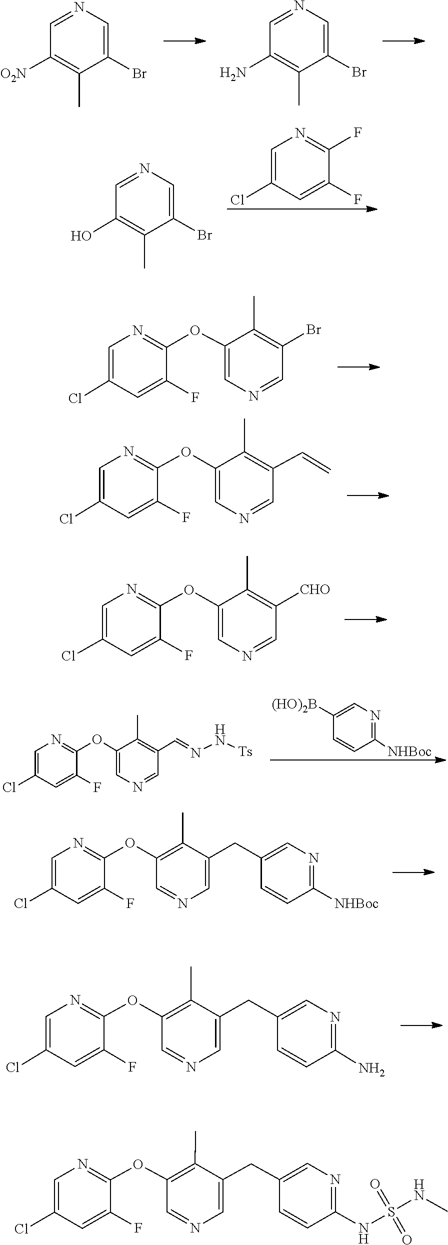

Step 1: To a solution of 3-bromo-4-methyl-5-nitro-pyridine (50 g, 230.39 mmol) in EtOH (1250 mL) and H₂O (250 mL) were added Fe (128.66 g, 2.30 mol) and NH₄Cl (36.97 g, 691.17 mmol). The mixture was stirred at 80° C. for 12 h. After cooling to room temperature, water (200 mL) was added to the mixture and the aqueous layer was extracted with EtOAc (100 mL×3). The combined organic phase was washed with brine (100 mL), dried over anhydrous Na₂SO₄, filtered and concentrated under reduced pressure. 5-bromo-4-methyl-pyridin-3-amine (34 g, 181.78 mmol). ¹H NMR (400 MHz, CDCl₃) δ=8.11 (s, 1H), 7.92 (s, 1H), 3.53 (br s, 2H), 2.26 (s, 3H)

Step 2: To a solution of 5-bromo-4-methyl-pyridin-3-amine (10 g, 53.47 mmol) in H₂SO₄ (50 mL) was added NaNO₂ (4.06 g, 58.81 mmol) in H₂O (25 mL) at 0° C. for 0.25 h. A solution of Cu(NO₃)₂ (258.35 g, 1.07 mol) in H₂O (400 mL) was added followed by Cu₂O (8.80 g, 61.49 mmol, 6.28 mL). The mixture was stirred at 25° C. for 11.75 h. NaOH solution (1.0 M) was added to the reaction mixture until pH=7. The reaction mixture was poured into water (20 mL) and extracted with EtOAc (100 mL×3). The combined organic layers were washed with brine (100 mL), dried over anhydrous Na₂SO₄, filtered and concentrated. The solid was purified by flash chromatography on silica gel (ethyl acetate in petroleum ether=0-30%) to give 5-bromo-4-methyl-pyridin-3-ol (6.3 g, 33.51 mmol). ¹H NMR (400 MHz, CDCl₃) δ=8.17 (s, 1H), 8.06 (s, 1H), 2.38 (s, 3H). LCMS Rₜ=0.463 min in 1.5 min chromatography, 5-95AB, ESI calcd. for C₆H₇BrNO [M+H]⁺190.0, found 189.9.

Step 3: To a solution of 5-bromo-4-methyl-pyridin-3-ol (2 g, 10.64 mmol) in DMAC (20 mL) were added 5-chloro-2,3-difluoro-pyridine (3.18 g, 21.27 mmol), CsF (2.42 g, 15.96 mmol, 588.28 μL) and TEA (3.23 g, 31.91 mmol, 4.44 mL). The mixture was stirred at 80° C. for 5 h. H₂O (30 mL) was added to the mixture. The aqueous phase was extracted with EtOAc (20 mL×3) and the combined organic phase was washed with brine (20 mL×3), dried over anhydrous Na₂SO₄, filtered and concentrated. The crude was purified by flash chromatography on silica gel (ethyl acetate in petroleum ether=0-10%) to give 2-[(5-bromo-4-methyl-3-pyridyl)oxy]-5-chloro-3-fluoro-pyridine (990 mg, 3.12 mmol). ¹H NMR (400 MHz, CDCl₃) δ=8.59 (s, 1H), 8.30 (s, 1H), 7.83 (d, J=2.0 Hz, 1H), 7.56 (dd, J=2.0, 8.8 Hz, 1H), 2.29 (s, 3H). ¹⁹F NMR (376.5 MHz, CDCl₃) δ=−134.165. LCMS Rₜ=0.946 min in 1.5 min chromatography, 5-95AB, ESI calcd. for C₁₁H₈BrClFN₂O [M+H]⁺318.9, found 318.7.

Step 4: To a solution of 2-[(5-bromo-4-methyl-3-pyridyl)oxy]-5-chloro-3-fluoro-pyridine (400 mg, 1.26 mmol) and 4,4,5,5-tetramethyl-2-vinyl-1,3,2-dioxaborolane (232.81 mg, 1.51 mmol, 256.40 μL) in dioxane (4 mL) and H₂O (0.4 mL) was added K₂CO₃ (522.29 mg, 3.78 mmol). The mixture was degassed and purged with N₂ for 3 times, then Pd(dppf)Cl₂ (92.17 mg, 125.97 μmol) was added to the mixture, degassed and purged with N₂ for 3 times. The mixture was stirred at 80° C. for 3 h. The mixture was blended with two batches (prepared from 400 mg and 50 mg 2-[(5-bromo-4-methyl-3-pyridyl)oxy]-5-chloro-3-fluoro-pyridine). Water (20 mL) was added and the mixture was extracted with EtOAc (30 mL×2) and the organic layer was dried over anhydrous Na₂SO₄, filtered and concentrated. The crude was purified by flash chromatography on silica gel (ethyl acetate in petroleum ether=0-10%) to give 3-[(5-chloro-3-fluoro-2-pyridyl)oxy]-4-methyl-5-vinyl-pyridine (500 mg, 1.89 mmol). ¹H NMR (400 MHz, CDCl₃) δ=8.55 (s, 1H), 8.28 (s, 1H), 7.83 (d, J=2.0 Hz, 1H), 7.54 (dd, J=2.4, 9.2 Hz, 1H), 6.85 (dd, J=10.8, 17.6 Hz, 1H), 5.76 (dd, J=1.2, 17.6 Hz, 1H), 5.48 (dd, J=1.2, 10.8 Hz, 1H), 2.19 (s, 3H). ¹⁹F NMR (376.5 MHz, CDCl₃) δ=−134.356. LCMS Rₜ=0.791 min in 1.5 min chromatography, 5-95AB, ESI calcd. for C₁₃H₁₁ClFN₂O [M+H]⁺265.1, found 264.9.

Step 5: To a solution of 3-[(5-chloro-3-fluoro-2-pyridyl)oxy]-4-methyl-5-vinyl-pyridine (450 mg, 1.70 mmol) in THF (13.5 mL) and H₂O (2.7 mL) were added K₂OsO₄·2H₂O (62.64 mg, 170.02 μmol) and NaIO₄ (1.45 g, 6.80 mmol, 376.84 μL). The mixture was stirred at 25° C. for 1 h. The mixture was blended with another batch prepared from 50 mg 3-[(5-chloro-3-fluoro-2-pyridyl)oxy]-4-methyl-5-vinyl-pyridine. The reaction was diluted with water (20 mL) and extracted with EtOAc (20 mL×3). The combined organic phase was dried over anhydrous Na₂SO₄, filtered and concentrated under reduced pressure. The crude was purified by flash chromatography on silica gel (ethyl acetate in petroleum ether=0-20%) to give 5-[(5-chloro-3-fluoro-2-pyridyl)oxy]-4-methyl-pyridine-3-carbaldehyde (400 mg, 1.50 mmol). 1H NMR (400 MHz, CDCl₃) δ=10.34 (s, 1H), 8.87 (s, 1H), 8.57 (s, 1H), 7.81 (d, J=2.4 Hz, 1H), 7.58 (dd, J=2.4, 9.2 Hz, 1H), 2.53 (s, 3H). ¹⁹F NMR (376.5 MHz, CDCl₃) δ=−134.181. LCMS Rₜ=0.811 min in 1.5 min chromatography, 5-95AB, ESI calcd. for C₁₂H₉ClFN₂O₂ [M+H]⁺267.0, found 266.9.

Step 6: To a solution of 5-[(5-chloro-3-fluoro-2-pyridyl)oxy]-4-methyl-pyridine-3-carbaldehyde (300 mg, 1.13 mmol) in MeOH (4 mL) was added 4-methylbenzenesulfonohydrazide (209.52 mg, 1.13 mmol). The mixture was stirred at 60° C. for 2 h. The mixture was blended with another batch prepared form 100 mg 5-[(5-chloro-3-fluoro-2-pyridyl)oxy]-4-methyl-pyridine-3-carbaldehyde. The mixture was concentrated directly. N-[(E)-[5-[(5-chloro-3-fluoro-2-pyridyl)oxy]-4-methyl-3-pyridyl]methyleneamino]-4-methyl-benzenesulfonamide (600 mg, 1.38 mmol). ¹H NMR (400 MHz, CDCl₃) δ=8.66 (s, 1H), 8.35 (s, 1H), 8.01 (s, 1H), 7.86 (d, J=8.4 Hz, 2H), 7.78 (d, J=2.4 Hz, 1H), 7.55 (dd, J=2.4, 9.2 Hz, 1H), 7.32 (d, J=8.4 Hz, 2H), 2.42 (s, 3H), 2.25 (s, 3H). ¹⁹F NMR (376.5 MHz, CDCl₃) δ=−134.211. LCMS Rₜ=0.867 min in 1.5 min chromatography, 5-95AB, ESI calcd. for C₁₉H₁₇ClFN₄O₃S [M+H]⁺435.1, found 434.9.

Step 7: To a solution of N-[(E)-[5-[(5-chloro-3-fluoro-2-pyridyl)oxy]-4-methyl-3-pyridyl]methyleneamino]-4-methyl-benzenesulfonamide (240 mg, 551.89 μmol) and [6-(tert-butoxycarbonylamino)-3-pyridyl]boronic acid (262.75 mg, 1.10 mmol) in dioxane (3 mL) was added K₂CO₃ (228.82 mg, 1.66 mmol). The mixture was stirred at 110° C. for 2 h. The mixture was blended with another batch prepared form 30 mg N-[(E)-[5-[(5-chloro-3-fluoro-2-pyridyl)oxy]-4-methyl-3-pyridyl]methyleneamino]-4-methyl-benzenesulfonamide. H₂O (30 mL) was added to the mixture. The aqueous phase was extracted with EtOAc (30 mL×3). The combined organic phase was washed with brine (20 mL×3), dried over anhydrous Na₂SO₄, filtered and concentrated. The crude was purified by flash chromatography on silica gel (ethyl acetate in petroleum ether=0-60%) to give tert-butyl N-[5-[[5-[(5-chloro-3-fluoro-2-pyridyl)oxy]-4-methyl-3-pyridyl]methyl]-2-pyridyl]carbamate (140 mg, 314.69 μmol). ¹H NMR (400 MHz, CDCl₃) δ=8.32 (s, 1H), 8.28 (s, 1H), 8.05 (d, J=2.0 Hz, 1H), 7.89 (d, J=8.4 Hz, 1H), 7.82 (d, J=2.0 Hz, 1H), 7.56-7.52 (m, 2H), 7.43 (dd, J=2.0, 8.8 Hz, 1H), 3.99 (s, 2H), 2.07 (s, 3H), 1.52 (s, 9H). ¹⁹F NMR (376.5 MHz, CDCl₃) δ=−134.319. LCMS Rₜ=0.840 min in 1.5 min chromatography, 5-95AB, ESI calcd. for C₂₂H₂₃ClFN₄O₃ [M+H]⁺445.1, found 445.1.

Step 8: To a solution of tert-butyl N-[5-[[5-[(5-chloro-3-fluoro-2-pyridyl)oxy]-4-methyl-3-pyridyl]methyl]-2-pyridyl]carbamate (120 mg, 269.73 μmol) in MeOH (2 mL) was added HCl/MeOH (4 M, 2.70 mL). The mixture was stirred at 25° C. for 2 h. The mixture was blended with another batch prepared from 20 mg N-[5-[[5-[(5-chloro-3-fluoro-2-pyridyl)oxy]-4-methyl-3-pyridyl]methyl]-2-pyridyl]carbamate. Sat. NaHCO₃ solution was added to the mixture until pH=7. The reaction mixture was extracted with EtOAc (30 mL×3) and the combined organic phase was washed with brine (50 mL), dried over anhydrous Na₂SO₄, filtered and concentrated. The crude was purified by flash chromatography on silica gel (dichloromethane in methanol=0-10%) to give 5-[[5-[(5-chloro-3-fluoro-2-pyridyl)oxy]-4-methyl-3-pyridyl]methyl]pyridin-2-amine (100 mg, 290.05 μmol). ¹H NMR (400 MHz, CDCl₃) δ=8.28 (d, J=6.4 Hz, 2H), 7.88 (s, 1H), 7.82 (d, J=2.0 Hz, 1H), 7.53 (dd, J=2.0, 9.2 Hz, 1H), 7.22 (dd, J=2.4, 8.8 Hz, 1H), 6.49 (d, J=8.8 Hz, 1H), 4.63 (br s, 2H), 3.90 (s, 2H), 2.08 (s, 3H). ¹⁹F NMR (376.5 MHz, CDCl₃) δ=−134.368. LCMS Rₜ=0.688 min in 1.5 min chromatography, 5-95AB, ESI calcd. for C₁₇H₁₅ClFN₄O [M+H]⁺345.1, found 345.1.

Step 9: To a solution of 5-[[5-[(5-chloro-3-fluoro-2-pyridyl)oxy]-4-methyl-3-pyridyl]methyl]pyridin-2-amine (80 mg, 232.04 μmol) in MeCN (1 mL) were added Py (220.25 mg, 2.78 mmol, 224.75 μL) and N-methylsulfamoyl chloride (60.13 mg, 464.08 umol). The mixture was stirred at 25° C. for 2 h. The mixture was blended with another batch prepared from 20 mg 5-[[5-[(5-chloro-3-fluoro-2-pyridyl)oxy]-4-methyl-3-pyridyl]methyl]pyridin-2-amine. The mixture was concentrated directly and purified by prep-HPLC (column: Boston Green ODS 150*30 mm*5 um; mobile phase: [water(FA)−ACN]; B %: 38%-68%, 6 min) to afford 5-[[5-[(5-chloro-3-fluoro-2-pyridyl)oxy]-4-methyl-3-pyridyl]methyl]-N-(methylsulfamoyl)pyridin-2-amine (23 mg, 52.53 umol, ¹H NMR (400 MHz, CDCl₃) δ=8.42-8.22 (m, 2H), 8.13 (d, J=1.2 Hz, 1H), 7.83 (d, J=2.4 Hz, 1H), 7.55 (dd, J=2.4, 9.2 Hz, 1H), 7.43 (dd, J=2.4, 8.8 Hz, 1H), 7.00 (d, J=8.8 Hz, 1H), 5.03 (br s, 1H), 4.01 (s, 2H), 2.73 (s, 3H), 2.13 (s, 3H). ¹⁹F NMR (376.5 MHz, CDCl₃) δ=−134.174. LCMS Rₜ=0.769 min in 1.5 min chromatography, 5-95AB, ESI calcd. for C₁₈H₁₈ClFN₅O₃S [M+H]⁺438.1, found 438.1. HPLC Rₜ=2.908 min in 8 min chromatography, 220 nm, purity 98.1%.

### Example 49: [(4-{[5-(4-chloro-2-fluorophenoxy)-4-methylpyridin-3-yl]methyl}-3-fluoropyridin-2-yl)sulfamoyl](methyl)amine

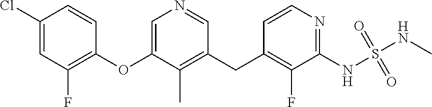 [SMILES: CNS(=O)(=O)Nc1nccc(Cc2cncc(Oc3ccc(Cl)cc3F)c2C)c1F]

### Route

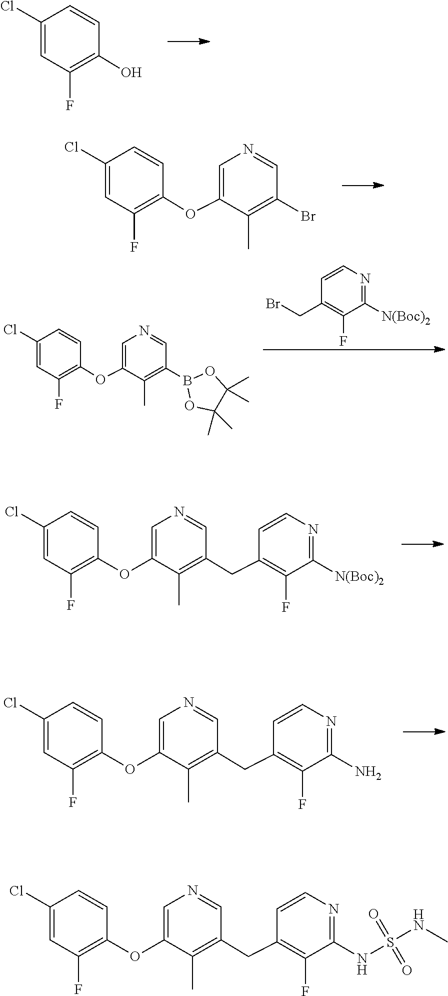

Step 1: To a stirred solution of 4-chloro-2-fluorophenol (700.00 mg, 4.777 mmol) and Cs₂CO₃ (3.11 g, 9.55 mmol) in DMF (10 mL) was added 3-bromo-5-fluoro-4-methylpyridine (907.61 mg, 4.777 mmol) at 0° C. under nitrogen atmosphere. The resulting mixture was stirred for 24 h at 120° C. under nitrogen atmosphere. Desired product could be detected by LCMS. The reaction mixture was diluted with water (50 mL). The resulting mixture was extracted with EA (3×50 mL). The combined organic layers were washed with brine (50 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 70% gradient in 30 min; detector, UV 254 nm. This resulted in 3-bromo-5-(4-chloro-2-fluorophenoxy)-4-methylpyridine (270 mg). LCMS: (ESI, m/z): [M+1]⁺=316.10. ¹H NMR (400 MHz, Chloroform-d) δ 8.47 (s, 1H), 7.98 (s, 1H), 7.24 (dd, J=10.2, 2.5 Hz, 1H), 7.11 (m, 1H), 6.91 (t, J=8.6 Hz, 1H), 2.42 (s, 3H). ¹⁹F NMR (376 MHz, Chloroform-d) δ −127.91.

Step 2: To a solution of 3-bromo-5-(4-chloro-2-fluorophenoxy)-4-methylpyridine (210 mg, 0.663 mmol, 1 equiv) and bis(pinacolato)diboron (252.70 mg, 0.995 mmol, 1.5 equiv) in dioxane (4 mL) were added AcOK (130.22 mg, 1.326 mmol, 2 equiv) and Pd(dppf)Cl₂ (48.54 mg, 0.066 mmol, 0.1 equiv). After stirring for 16 h at 100° C. under a nitrogen atmosphere, the resulting mixture was concentrated under reduced pressure. This resulted in 3-(4-chloro-2-fluorophenoxy)-4-methyl-5-(4,4,5,5-tetramethyl-1,3,2-dioxaborolan-2-yl)pyridine.

Step 3: To a solution of 3-(4-chloro-2-fluorophenoxy)-4-methyl-5-(4,4,5,5-tetramethyl-1,3,2-dioxaborolan-2-yl)pyridine (200 mg, 0.55 mmol, 1 equiv) and tert-butyl N-[4-(bromomethyl)-3-fluoropyridin-2-yl]-N-(tert-butoxycarbonyl) carbamate (111.45 mg, 0.275 mmol, 0.5 equiv) in dioxane (2 mL) and H₂O (0.2 mL) were added K₂CO₃ (152.02 mg, 1.1 mmol, 2 equiv) and Pd(dppf)Cl₂ (40.24 mg, 0.055 mmol, 0.1 equiv). After stirring for 2 h at 80° C. under a nitrogen atmosphere, the reaction mixture was concentrated under reduced pressure. The residue was purified by silica gel column chromatography, eluted with PE/EA (6:1) to afford tert-butyl N-(tert-butoxycarbonyl)-N-(4-{[5-(4-chloro-2-fluorophenoxy)-4-methylpyridin-3-yl]methyl}-3-fluoropyridin-2-yl) carbamate (200 mg). LCMS: (ESI, m/z): [M+1]⁺=562.05. ¹H NMR (400 MHz, Chloroform-d) δ 8.21 (m, 2H), 8.03 (s, 1H), 7.25-7.19 (m, 1H), 7.12 (m, 1H), 7.05-6.86 (m, 2H), 4.11 (m, 2H), 2.23 (s, 3H), 1.42 (m, 18H). ¹⁹F NMR (376 MHz, Chloroform-d) δ −127.90, −130.89

Step 4: To a stirred solution of tert-butyl N-(tert-butoxycarbonyl)-N-(4-{[5-(4-chloro-2-fluorophenoxy)-4-methylpyridin-3-yl]methyl}-3-fluoropyridin-2-yl) carbamate (200 mg, 0.356 mmol, 1 equiv) in DCM (8 mL) were added TFA (2 mL) at 0° C. The resulting mixture was stirred for 1 h at room temperature. Desired product could be detected by LCMS. The reaction mixture was concentrated under reduced pressure. The residue was a basified to pH 10 with sat. NaHCO₃ (aq.). The resulting mixture was extracted with EA (3×30 mL). The combined organic layers were washed with brine (30 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in Water (10 mmol/L NH₄HCO₃), 5% to 40% gradient in 30 min; detector, UV 254 nm. This resulted in 4-{[5-(4-chloro-2-fluorophenoxy)-4-methylpyridin-3-yl]methyl}-3-fluoropyridin-2-amine (40 mg). LCMS: (ESI, m/z): [M+1]⁺=362.00. ¹H NMR (400 MHz, Chloroform-d) δ 8.22 (s, 1H), 8.04 (s, 1H), 7.74 (d, J=5.2 Hz, 1H), 7.23 (dd, J=10.3, 2.5 Hz, 1H), 7.08 (m, 1H), 6.86 (t, J=8.7 Hz, 1H), 6.27 (t, J=5.1 Hz, 1H), 4.63 (s, 2H), 4.00 (s, 2H), 2.22 (s, 3H). ¹⁹F NMR (377 MHz, Chloroform-d) δ −128.42, −145.29.

Step 5: To a stirred solution of 4-{[5-(4-chloro-2-fluorophenoxy)-4-methylpyridin-3-yl]methyl}-3-fluoropyridin-2-amine (40 mg, 0.111 mmol, 1 equiv) and pyridine (87.46 mg, 1.110 mmol, 10 equiv) in DMA (1 mL) was added N-methylsulfamoyl chloride (15.76 mg, 0.122 mmol, 1.10 equiv) in DMA (1 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in Water (10 mmol/L NH₄HCO₃), 5% to 60% gradient in 30 min; detector, UV 254 nm. This resulted in [(4-{[5-(4-chloro-2-fluorophenoxy)-4-methylpyridin-3-yl]methyl}-3-fluoropyridin-2-yl)sulfamoyl](methyl)amine (25.3 mg). LCMS: (ESI, m/z): [M+1]⁺=455.05. ¹H NMR (400 MHz, Methanol-d₄) δ 8.19 (s, 1H), 7.99 (d, J=5.2 Hz, 1H), 7.96 (s, 1H), 7.40 (dd, J=10.6, 2.5 Hz, 1H), 7.21 (m, 1H), 7.04 (t, J=8.8 Hz, 1H), 6.73 (t, J=5.1 Hz, 1H), 4.19 (s, 2H), 2.63 (s, 3H), 2.27 (s, 3H). ¹⁹F NMR (376 MHz, Methanol-d₄) δ −130.57, −142.30.

### Example 50: {[4-({5-[(4-chloro-2-fluorophenyl)methyl]-4-methylpyridin-3-yl}methyl)-3-fluoropyridin-2-yl]sulfamoyl}(methyl)amine

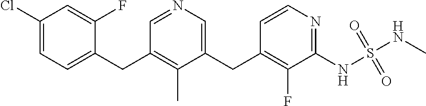 [SMILES: CNS(=O)(=O)Nc1nccc(Cc2cncc(Cc3ccc(Cl)cc3F)c2C)c1F]

### Route

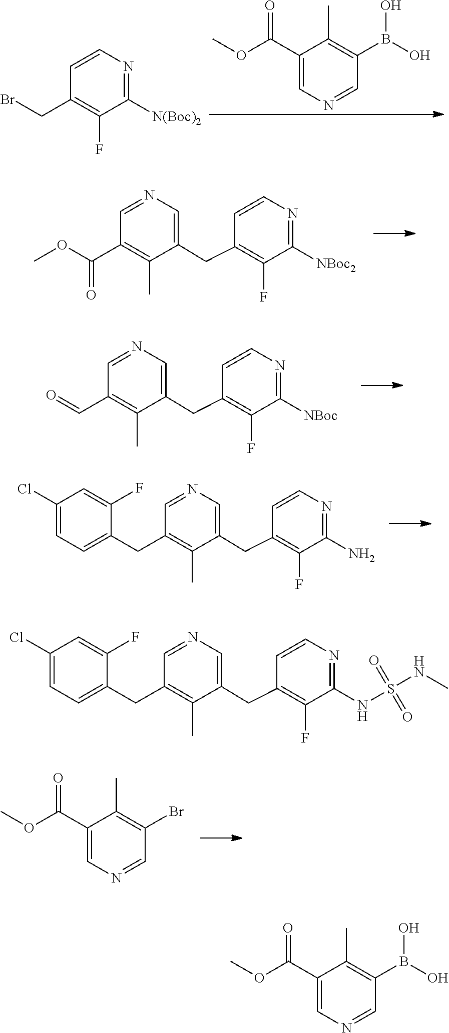

Step 1: To a mixture of tert-butyl N-[4-(bromomethyl)-3-fluoropyridin-2-yl]-N-(tert-butoxycarbonyl) carbamate (1 g, 2.468 mmol) and 5-(methoxycarbonyl)-4-methylpyridin-3-ylboronic acid (721.68 mg, 3.702 mmol) in dioxane (10 mL) and H₂O (2 mL) were added K₂CO₃ (1.023 g, 7.404 mmol) and Pd(dppf)Cl₂ (180.55 mg, 0.247 mmol) under nitrogen atmosphere. The resulting mixture was stirred for 1 h at 80° C. Desired product could be detected by LCMS. The reaction mixture was concentrated under reduced pressure. The residue was purified by silica gel column chromatography, eluted with PE/EA (1:1) to afford methyl 5-({2-[bis(tert-butoxycarbonyl)amino]-3-fluoropyridin-4-yl}methyl)-4-methylpyridine-3-carboxylate (620 mg). LCMS: [M+1]⁺=476.51H NMR (400 MHz, Chloroform-d) δ 8.97 (s, 1H), 8.49 (s, 1H), 8.18 (d, J=5.0 Hz, 1H), 6.84 (t, J=5.0 Hz, 1H), 4.13 (s, 2H), 3.94 (s, 3H), 2.46 (s, 3H), 1.41 (s, 18H). ¹⁹F NMR (377 MHz, Chloroform-d) δ −130.83.

Step 2: To a stirred solution of methyl 5-({2-[bis(tert-butoxycarbonyl)amino]-3-fluoropyridin-4-yl}methyl)-4-methylpyridine-3-carboxylate (414 mg, 0.871 mmol, 1 equiv) in MeOH (5 mL) were added CaCl₂) (483.11 mg, 4.355 mmol, 5 equiv) and NaBH₄ (329.36 mg, 8.710 mmol, 10 equiv) in portions at 0° C. under nitrogen atmosphere. The resulting reaction was stirred for 16 h at room temperature. Desired product could be detected by LCMS. The reaction was quenched by the addition of water/ice (5 mL) at 0° C. The resulting mixture was extracted with EA (3× 20 mL). The combined organic layers were washed with brine (20 mL), dried over anhydrous Na₂SO₄. After filtration, the filtrate was concentrated under reduced pressure. The residue was purified by silica gel column chromatography, eluted with DCM/MeOH (10:1) to afford tert-butyl N-(3-fluoro-4-{[5-(hydroxymethyl)-4-methylpyridin-3-yl]methyl}pyridin-2-yl) carbamate (206 mg). LCMS: [M+1]⁺=348.30. ¹H NMR (400 MHz, Chloroform-d) δ 8.48 (s, 1H), 8.34 (s, 1H), 8.07 (d, J=5.1 Hz, 1H), 6.96 (s, 1H), 6.59 (t, J=5.1 Hz, 1H), 4.75 (s, 2H), 4.05 (s, 2H), 2.25 (s, 3H), 1.53 (s, 9H). ¹⁹F NMR (376 MHz, Chloroform-d) δ −137.12.

Step 3: To a stirred solution of tert-butyl N-(3-fluoro-4-{[5-(hydroxymethyl)-4-methylpyridin-3-yl]methyl}pyridin-2-yl) carbamate (150 mg, 0.432 mmol, 1 equiv) in DCM (3 mL) was added MnO₂ (187.69 mg, 2.160 mmol, 5 equiv) at room temperature under air atmosphere. The resulting mixture was stirred for 16 h at 50° C. Desired product could be detected by LCMS. The reaction mixture was filtered, the filter cake was washed with DCM (3×10 mL). The filtrate was concentrated under reduced pressure. This resulted in tert-butyl N-{3-fluoro-4-[(5-formyl-4-methylpyridin-3-yl)methyl]pyridin-2-yl}carbamate (126 mg). LCMS: [M+1]⁺=346.05. ¹H NMR (400 MHz, Chloroform-d) δ 10.31 (s, 1H), 8.88 (s, 1H), 8.57 (s, 1H), 8.09 (d, J=5.0 Hz, 1H), 6.97 (s, 1H), 6.59 (t, J=5.1 Hz, 1H), 4.11 (s, 2H), 2.56 (s, 3H), 1.53 (s, 9H). ¹⁹F NMR (376 MHz, Chloroform-d) δ −136.74.

Step 4: To a stirred solution of tert-butyl N-{3-fluoro-4-[(5-formyl-4-methylpyridin-3-yl)methyl]pyridin-2-yl}carbamate (100 mg, 0.290 mmol, 1 equiv) in MeOH (2 mL) was added 4-toluenesulfonyl hydrazide (64.71 mg, 0.348 mmol, 1.2 equiv) in portions at 0° C. under air atmosphere. The resulting mixture was stirred for 2 h at room temperature. Desired product could be detected by LCMS. The reaction mixture was concentrated under reduced pressure. The residue was purified by silica gel column chromatography, eluted with DCM/MeOH (10:1) to afford tert-butyl N-[3-fluoro-4-({4-methyl-5-[(1Z)-[(4-methylbenzenesulfonamido) imino]methyl]pyridin-3-yl}methyl)pyridin-2-yl]carbamate (140 mg). LCMS: [M+1]⁺=514.15. ¹H NMR (400 MHz, Methanol-d₄) δ 8.62 (s, 1H), 8.33 (s, 1H), 8.13 (s, 1H), 8.02 (d, J=5.1 Hz, 1H), 7.85-7.81 (m, 2H), 7.40 (d, J=8.1 Hz, 2H), 6.82 (t, J=5.1 Hz, 1H), 4.18 (s, 2H), 2.43 (s, 3H), 2.30 (s, 3H), 1.52 (s, 9H). ¹⁹F NMR (376 MHz, Methanol-d₄) δ −134.82.

Step 5: To a mixture of tert-butyl N-[3-fluoro-4-({4-methyl-5-[(1Z)-[(4-methylbenzenesul fonamido)imino]methyl]pyridin-3-yl}methyl)pyridin-2-yl]carbamate (140 mg, 0.273 mmol, 1 equiv) and K₂CO₃ (45.21 mg, 0.328 mmol, 1.2 equiv) in dioxane (1 mL) was added 4-chloro-2-fluorophenylboronic acid (475.29 mg, 2.730 mmol, 10 equiv) at room temperature under nitrogen atmosphere. The resulting mixture was stirred for 1 h at 100° C. Desired product could be detected by LCMS. The reaction mixture was concentrated under reduced pressure. The residue was purified by reversed-phase flash chromatography with the following conditions: column, C18 silica gel; mobile phase, ACN in water (10 mmol/L NH₄HCO₃), 20% to 80% gradient in 30 min; detector, UV 254 nm. This resulted in tert-butyl N-[4-({5-[(4-chloro-2-fluorophenyl)methyl]-4-methylpyridin-3-yl}methyl)-3-fluoropyridin-2-yl]carbamate (25 mg). LCMS: [M+1]⁺=460.451H NMR (400 MHz, Chloroform-d) δ 8.31 (d, J=6.8 Hz, 2H), 8.07 (d, J=5.1 Hz, 1H), 7.16-7.07 (m, 1H), 7.07-7.00 (m, 1H), 6.92 (s, 1H), 6.84 (t, J=8.2 Hz, 1H), 6.57 (t, J=5.1 Hz, 1H), 4.03 (s, 2H), 3.97 (s, 2H), 2.08 (s, 3H), 1.53 (s, 9H). ¹⁹F NMR (376 MHz, Chloroform-d) δ −114.56, −137.29.

Step 6: To a stirred solution of tert-butyl N-[4-({5-[(4-chloro-2-fluorophenyl)methyl]-4-methylpyridin-3-yl}methyl)-3-fluoropyridin-2-yl]carbamate (20 mg, 0.04 mmol, 1 equiv) in DCM (1.2 mL) was added TFA (0.3 mL) dropwise at 0° C. under air atmosphere. The resulting mixture was stirred for 1 h at room temperature. Desired product could be detected by LCMS. The reaction mixture was concentrated under reduced pressure. The residue was basified to pH 10 with sat. NaHCO₃ (aq.). The resulting mixture was extracted with EA (3×10 mL). The combined organic layers were washed with brine (10 mL), dried over anhydrous Na₂SO₄. After filtration, the filtrate was concentrated under reduced pressure. The residue was purified by Prep-TLC DCM/MeOH (10:1) to afford 4-({5-[(4-chloro-2-fluorophenyl)methyl]-4-methylpyridin-3-yl}methyl)-3-fluoropyridin-2-amine (16 mg). LCMS: [M+1]⁺=360.25. ¹H NMR (400 MHz, Chloroform-d) δ 8.31 (s, 2H), 7.71 (d, J=5.2 Hz, 1H), 7.16-7.01 (m, 2H), 6.84 (t, J=8.2 Hz, 1H), 6.21 (t, J=5.1 Hz, 1H), 4.77-4.61 (m, 2H), 3.97 (s, 4H), 2.10 (s, 3H). ¹⁹F NMR (377 MHz, Chloroform-d) δ −114.54, −145.18.

Step 7: To a stirred solution of 4-({5-[(4-chloro-2-fluorophenyl)methyl]-4-methylpyridin-3-yl}methyl)-3-fluoropyridin-2-amine (15 mg, 0.042 mmol, 1 equiv) and pyridine (32.98 mg, 0.420 mmol, 10 equiv) in DMA (1 mL) was added N-methylsulfamoyl chloride (27.01 mg, 0.210 mmol, 5 equiv) in DMA (0.2 mL) dropwise at 0° C. under air atmosphere. The resulting mixture was stirred for 1 h at room temperature. Desired product could be detected by LCMS. The reaction mixture was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in {[4-({5-[(4-chloro-2-fluorophenyl)methyl]-4-methylpyridin-3-yl}methyl)-3-fluoropyridin-2-yl]sulfamoyl}(methyl)amine (10.3 mg). CMS: [M+1]⁺=453.05. ¹H NMR (300 MHz, Methanol-d₄) δ 8.25 (s, 2H), 7.94 (d, J=5.2 Hz, 1H), 7.25-7.16 (m, 1H), 7.16-7.08 (m, 1H), 7.02 (t, J=8.2 Hz, 1H), 6.63 (t, J=5.2 Hz, 1H), 4.13 (s, 2H), 4.07 (s, 2H), 2.62 (s, 3H), 2.17 (s, 3H). ¹⁹F NMR (282 MHz, Methanol-d₄) δ −116.244, −142.322.

Boronate: To a mixture of methyl 5-bromo-4-methylpyridine-3-carboxylate (3.6 g, 15.648 mmol, 1 equiv) and bis(pinacolato)diboron (5.96 g, 23.472 mmol, 1.5 equiv) in dioxane (30 mL) were added AcOK (4.61 g, 46.944 mmol, 3 equiv) and Pd(dppf)Cl₂ (1.14 g, 1.565 mmol, 0.1 equiv) at room temperature under nitrogen atmosphere. The resulting mixture was stirred for 16 h at 110° C. under nitrogen atmosphere. Desired product could be detected by LCMS. The reaction mixture was diluted with H₂O (50 mL). The resulting mixture was extracted with EA (3×50 mL). The combined organic layers were washed with brine (50 mL), dried over anhydrous Na₂SO₄. After filtration, the filtrate was concentrated under reduced pressure. The residue was acidified to pH 6 with FA. The resulting mixture was purified by reversed-phase flash chromatography with the following conditions: column, C18 silica gel; mobile phase, ACN in water (0.1% FA), 10% to 50% gradient in 30 min; detector, UV 254/220 nm. This resulted in 5-(methoxycarbonyl)-4-methylpyridin-3-ylboronic acid (2.1 g). LCMS: [M+1]⁺=196.2

### Example 51: 3-fluoro-4-[[5-[2-fluoro-4-(trifluoromethyl) phenoxy]-4-methyl-3-pyridyl]methyl]-N-(methylsulfamoyl)pyridin-2-amine

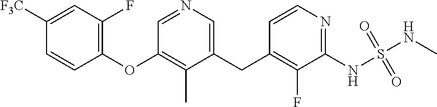 [SMILES: CNS(=O)(=O)Nc1nccc(Cc2cncc(Oc3ccc(C(F)(F)F)cc3F)c2C)c1F]

### Route

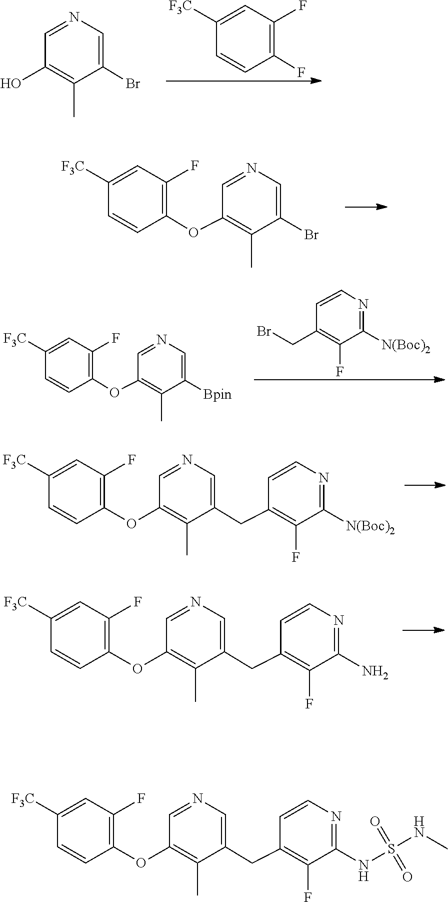

Step 1: A mixture of 5-bromo-4-methyl-pyridin-3-ol (950 mg, 5.05 mmol), 1,2-difluoro-4-(trifluoromethyl)benzene (2.02 g, 11.12 mmol) and t-BuOK (1.13 g, 10.11 mmol) in DMF (30 mL) was stirred at 100° C. for 36 h. The mixture was poured into H₂O (100 mL) at 0° C., then stirred for 10 mins and extracted with EtOAc (100 mL×3). The combined organic layers were washed with brine (200 mL), dried over anhydrous Na₂SO₄, filtered and concentrated. The residue was purified by flash chromatography on silica gel (EtOAc in PE=0-20%) to give 3-bromo-5-[2-fluoro-4-(trifluoromethyl) phenoxy]-4-methyl-pyridine (440 mg, 1.26 mmol). ¹H NMR (400 MHz, CDCl₃) δ=8.54 (s, 1H), 8.10 (s, 1H), 7.49 (dd, J=1.6, 10.4 Hz, 1H), 7.38 (d, J=8.8 Hz, 1H), 6.96 (t, J=8.4 Hz, 1H), 2.40 (s, 3H). ¹⁹F NMR (376.5 MHz, CDCl₃) δ=−62.215, −129.175. LCMS Rₜ=2.298 min 3.0 min chromatography, 10-80CD, ESI calcd. for C₁₃H₉BrF₄NO [M+H]⁺350.0, found 350.0.

Step 2: To a solution of 3-bromo-5-[2-fluoro-4-(trifluoromethyl) phenoxy]-4-methyl-pyridine (340 mg, 971.13 mmol) and 4,4,5,5-tetramethyl-2-(4,4,5,5-tetramethyl-1,3,2-dioxaborolan-2-yl)-1,3,2-dioxaborolane (517.88 mg, 2.04 mmol) in dioxane (12 mL) were added KOAc (285.93 mg, 2.91 mmol) and Pd(dppf)Cl₂ (213.18 mg, 291.34 mmol). The resulting mixture was degassed and purged with N₂ for 3 times. The reaction mixture was stirred under N₂ at 100° C. for 3 h. After cooling to room temperature, the mixture was concentrated under reduced pressure to remove 1,4-Dioxane. Then the reaction mixture was poured into water (30 mL) and extracted with EtOAc (30 mL×3). The combined organic layers were washed with brine (50 mL), dried over anhydrous Na₂SO₄, filtered and concentrated. The residue was purified by flash chromatography on silica gel (EtOAc in PE=0-40%) to give 3-[2-fluoro-4-(trifluoromethyl) phenoxy]-4-methyl-5-(4,4,5,5-tetramethyl-1,3,2-dioxaborolan-2-yl)pyridine (380 mg, 956.77 mmol). ¹H NMR (400 MHz, CDCl₃) δ=8.74 (s, 1H), 8.24 (s, 1H), 7.47 (dd, J=2.0, 10.8 Hz, 1H), 7.32 (d, J=8.8 Hz, 1H), 6.83 (t, J=8.4 Hz, 1H), 2.48 (s, 3H), 1.37 (s, 12H). ¹⁹F NMR (376.5 MHz, CDCl₃) δ=−62.120, −129.811. LCMS Rₜ=0.744 min 1.5 min chromatography, 5-95AB, ESI calcd. for C₁₃H₁₁BF₄NO₃ [M−C₆H₉]⁺316.1, found 315.9.

Step 3: To a solution of 3-[2-fluoro-4-(trifluoromethyl) phenoxy]-4-methyl-5-(4,4,5,5-tetramethyl-1,3,2-dioxaborolan-2-yl)pyridine (380 mg, 956.77 mmol) and tert-butyl N-[4-(bromomethyl)-3-fluoro-2-pyridyl]-N-tert-butoxycarbonyl-carbamate (465.29 mg, 1.15 mmol) in toluene (8 mL), EtOH (4 mL) and H₂O (1.6 mL) were added Pd(PPh₃)₄ (221.12 mg, 191.35 mmol) and Na₂CO₃ (304.22 mg, 2.87 mmol). The mixture was degassed and purged with N₂ for 3 times. The reaction mixture was stirred at 80° C. for 12 h. The reaction mixture was concentrated to remove the solvent. The residue was diluted with water (10 mL) and extracted with EtOAc (10 mL×2). The combined organic layers were washed with brine (10 mL), dried over anhydrous Na₂SO₄, filtered and concentrated. The residue was purified by flash chromatography on silica gel (EtOAc in PE=10-100%) to give tert-butyl N-tert-butoxycarbonyl-N-[3-fluoro-4-[[5-[2-fluoro-4-(trifluoromethyl) phenoxy]-4-methyl-3-pyridyl]methyl]-2-pyridyl]carbamate (100 mg, 167.91 mmol). ¹H NMR (400 MHz, CDCl₃) δ=8.32-8.19 (m, 2H), 8.15 (s, 1H), 7.48 (d, J=10.4 Hz, 1H), 7.37 (d, J=8.4 Hz, 1H), 6.97-6.89 (m, 2H), 4.12 (s, 2H), 2.19 (s, 3H), 1.42 (s, 18H). LCMS Rₜ=1.09 min 1.5 min chromatography, 5-95CD, ESI calcd. for C₂₉H₃₁F₅N₃O₅ [M+H]⁺596.2, found 596.2. C₂₉H₃₀F₅N₃O₅Na [M+Na]⁺618.2, found 618.2.

Step 4: To a solution of tert-butyl N-tert-butoxycarbonyl-N-[3-fluoro-4-[[5-[2-fluoro-4-(trifluoromethyl) phenoxy]-4-methyl-3-pyridyl]methyl]-2-pyridyl]carbamate (100 mg, 167.91 mmol) in MeOH (2 mL) was added HCl/MeOH (4 M, 8.00 mmol, 2 mL) at 25° C. The reaction mixture was stirred at 25° C. for 12 h. The reaction mixture was concentrated. Then diluted with NH₃/MeOH (7 M, 10 mL×2) and concentrated under vacuum. The residue was purified by flash chromatography on silica gel (EtOAc in PE=0-80%) to give 3-fluoro-4-[[5-[2-fluoro-4-(trifluoromethyl) phenoxy]-4-methyl-3-pyridyl]methyl]pyridin-2-amine (36.6 mg, 92.58 mmol). LCMS Rₜ=0.764 min 1.5 min chromatography, 5-95AB, ESI calcd. for C₁₉H₁₅F₅N₃O [M+H]⁺396.1, found 396.1.

Step 5: To a solution of 3-fluoro-4-[[5-[2-fluoro-4-(trifluoromethyl) phenoxy]-4-methyl-3-pyridyl]methyl]pyridin-2-amine (36.6 mg, 92.58 mmol) in MeCN (1.5 mL) was added pyridine (219.70 mg, 2.78 mmol, 224.18 mL) and N-methylsulfamoyl chloride (179.93 mg, 1.39 mmol) at 25° C. The reaction mixture was stirred at 25° C. for 4 h. The reaction mixture was concentrated. The residue was purified by pre-HPLC (column: Boston Prime C18 150×30 mm×5 um; mobile phase: [water(NH₃H₂O+NH₄HCO₃)−ACN]; B %: 35%-65%, 7 min) to give 3-fluoro-4-[[5-[2-fluoro-4-(trifluoromethyl) phenoxy]-4-methyl-3-pyridyl]methyl]-N-(methylsulfamoyl)pyridin-2-amine (8.8 mg, 18.02 mmol). ¹H NMR (400 MHz, CDCl₃) δ=8.26 (s, 1H), 8.15 (s, 1H), 7.98 (d, J=4.8 Hz, 1H), 7.49 (dd, J=1.6, 10.4 Hz, 1H), 7.38 (d, J=8.8 Hz, 1H), 7.21 (s, 1H), 6.95 (t, J=8.0 Hz, 1H), 6.61 (t, J=5.2 Hz, 1H), 5.46 (s, 1H), 4.08 (s, 2H), 2.78 (d, J=5.2 Hz, 3H), 2.21 (s, 3H). ¹⁹F NMR (376.5 MHz, CDCl₃) δ=−62.178, −129.406, −142.798. LCMS Rₜ=0.823 min 1.5 min chromatography, 5-95AB, ESI calcd. for C₂₀H₁₈F₅N₄O₃S [M+H]⁺489.1, found 489.1. HPLC Rₜ=4.020 min in 8 min chromatography, 220 nm, purity 96.7%.

### Example 52: N-(4-chloro-2-fluoro-phenyl)-5-[[3-fluoro-2-(methylsulfamoylamino)-4-pyridyl]methyl]-4-methyl-pyridazin-3-amine

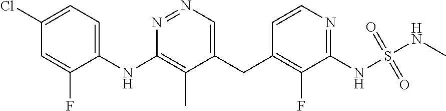 [SMILES: CNS(=O)(=O)Nc1nccc(Cc2cnnc(Nc3ccc(Cl)cc3F)c2C)c1F]

### Route

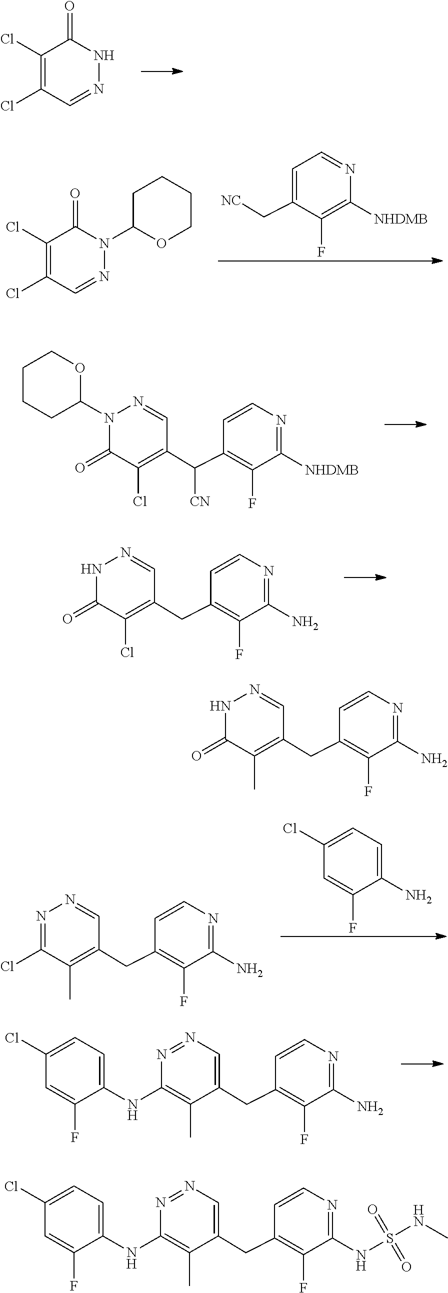

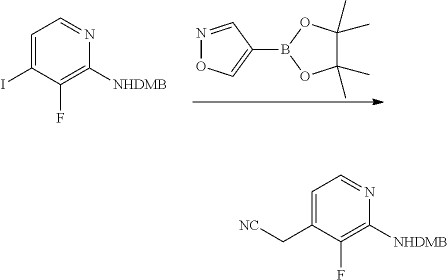

Intermediate Synthesis: A mixture of N-[(2,4-dimethoxyphenyl)methyl]-3-fluoro-4-iodo-pyridin-2-amine (3 g, 7.73 mmol), 4-(4,4,5,5-Tetramethyl-1,3,2-dioxaborolan-2-yl) isoxazole (2.14 g, 10.99 mmol), Pd(dppf)Cl₂ (565.50 mg, 772.85 μmol) and Na₂CO₃ (4.10 g, 38.64 mmol) in H₂O (4 mL) and dioxane (20 mL) was stirred at 85° C. for 16 hours under N₂. The mixture was cooled to 25° C. and then filtered. The filtrate was concentrated. The residue was purified by flash chromatography on silica gel (Ethyl acetate in Petroleum ether=0-20%) to give 2-[2-[(2,4-dimethoxyphenyl)methylamino]-3-fluoro-4-pyridyl]acetonitrile (1.6 g, 5.31 mmol). ¹H NMR (400 MHz, DMSO-d₆) δ=7.77 (d, J=5.2 Hz, 1H), 7.10-6.98 (m, 2H), 6.56-6.51 (m, 2H), 6.42 (dd, J=2.4, 8.4 Hz, 1H), 4.45 (d, J=5.6 Hz, 2H), 4.05 (s, 2H), 3.80 (s, 3H), 3.72 (s, 3H). ¹⁹F NMR (376.5 MHz, DMSO-d₆) δ=−145.380 ppm.

Step 1: To a solution of 4,5-dichloro-1H-pyridazin-6-one (10 g, 60.61 mmo) and 3,4-dihydro-2H-pyran (6.37 g, 75.77 mmol, 6.93 mL) in THF (100 mL) was added 4-methylbenzenesulfonic acid (1.04 g, 6.06 mmol) under N₂. The mixture was stirred at 66° C. stirred for 16 h. The mixture was concentrated. The residue was purified by flash chromatography on silica gel (EtOAc in PE=0-7%) to give 4,5-dichloro-2-tetrahydropyran-2-yl-pyridazin-3-one (6.6 g, 26.50 mmol). ¹H NMR (400 MHz, CDCl₃) δ=7.84 (s, 1H), 6.01 (dd, J=1.6, 10.4 Hz, 1H), 4.30-4.06 (m, 1H), 3.87-3.67 (m, 1H), 2.21-1.98 (m, 2H), 1.80-1.61 (m, 4H).

Step 2: To a solution of 2-[2-[(2,4-dimethoxyphenyl)methylamino]-3-fluoro-4-pyridyl]acetonitrile (1.6 g, 5.31 mmol) in DMF (16 mL) was added NaH (531.01 mg, 13.28 mmol, 60% purity) in portions at 0° C. under N₂. The mixture was stirred at 0° C. for 0.5 h. 4,5-dichloro-2-tetrahydropyran-2-yl-pyridazin-3-one (1.59 g, 6.37 mmol) was added at 0° C. The mixture was stirred at 25° C. for 1 h. The mixture was quenched with sat. NH₄Cl (50 mL). The aqueous layer was extracted with EtOAc (50 mL×3), the organic layer was washed with brine (100 mL×4), dried over anhydrous Na₂SO₄, filtered and concentrated. The residue was purified by flash chromatography on silica gel (Ethyl acetate in Petroleum ether=0-25%) to give 2-(5-chloro-6-oxo-1-(tetrahydro-2H-pyran-2-yl)-1,6-dihydropyridazin-4-yl)-2-(2-((2,4-dimethoxybenzyl)amino)-3-fluoropyridin-4-yl) acetonitrile (1.9 g, 3.70 mmol). 1H NMR (400 MHz, CD₃CN) δ=7.95 (d, J=7.2 Hz, 1H), 7.87 (dd, J=1.6, 5.2 Hz, 1H), 7.14 (d, J=8.0 Hz, 1H), 6.72-6.66 (m, 1H), 6.52 (d, J=2.4 Hz, 1H), 6.44-6.37 (m, 1H), 5.94-5.87 (m, 1H), 5.76 (br s, 1H), 5.70 (d, J=1.6 Hz, 1H), 4.51 (d, J=6.0 Hz, 2H), 4.05-3.96 (m, 1H), 3.81 (s, 3H), 3.75 (s, 3H), 3.70-3.61 (m, 1H), 2.13-1.99 (m, 2H), 1.74-1.51 (m, 4H). ¹⁹F NMR (376.5 MHz, CD₃CN) δ=−144.522 ppm.

Step 3: To a solution of 2-(5-chloro-6-oxo-1-tetrahydropyran-2-yl-pyridazin-4-yl)-2-[2-[(2,4-dimethoxyphenyl)methylamino]-3-fluoro-4-pyridyl]acetonitrile (1.9 g, 3.70 mmol) in Water (2 mL) and HCl (8 mL) were added AcOH (2.10 g, 34.97 mmol, 2 mL). The mixture was stirred at 110° C. for 2 h. The mixture was cooled to 25° C. and a brown solid precipitated from the solution, filtered and washed with water (10 mL×3), the filtrate was quenched with sat. NaHCO₃ (100 mL) and extracted with EtOAc (30 mL×5). The combined organic layers were washed with brine (20 mL), dried over anhydrous Na₂SO₄, filtered and concentrated under reduced pressure to give 4-[(2-amino-3-fluoro-4-pyridyl)methyl]-5-chloro-1H-pyridazin-6-one (941.40 mg, 3.70 mmol). ¹H NMR (400 MHz, DMSO-d₆) δ=7.82 (s, 1H), 7.65 (d, J=5.2 Hz, 1H), 6.37 (t, J=4.8 Hz, 1H), 6.20 (s, 2H), 3.95 (s, 2H). ¹⁹F NMR (400 MHz, DMSO-d₆) δ=−144.172 ppm.

Step 4: A mixture of 5-((2-amino-3-fluoropyridin-4-yl)methyl)-4-chloropyridazin-3 (2H)-one (900 mg, 3.53 mmol), Pd(dppf)Cl₂ (387.91 mg, 530.14 μmol), methylboronic acid (423.13 mg, 7.07 mmol) and K₂CO₃ (1.47 g, 10.60 mmol) in dioxane (12 mL) was stirred at 110° C. stirred for 1.5 h under N₂. The mixture was cooled to 25° C. and then filtered. The filtrate was concentrated. The residue was purified by flash chromatography on silica gel (MeOH in DCM=0-8%) to give 5-((2-amino-3-fluoropyridin-4-yl)methyl)-4-methylpyridazin-3 (2H)-one (827.83 mg, 3.53 mmol). ¹H NMR (400 MHz, DMSO-d₆) δ=12.93 (br s, 1H), 7.69-7.64 (m, 2H), 6.35 (t, J=4.8 Hz, 1H), 6.19 (s, 2H), 3.84 (s, 2H), 2.01 (s, 3H). ¹⁹F NMR (400 MHz, DMSO-d₆) δ=−144.347 ppm.

Step 5: A solution of 4-[(2-amino-3-fluoro-4-pyridyl)methyl]-5-methyl-1H-pyridazin-6-one (800 mg, 3.42 mmol) in POCl₃ (8 mL) was stirred at 100° C. stirred for 1 h under N₂. The mixture was cooled to 25° C. The mixture was slowly added into water (50 mL), then 30%. NaOH (aq) was added dropwise adjust to pH=7, extracted with EtOAc (25 mL×5). The combined organic layers were washed with brine (50 mL), dried over anhydrous Na₂SO₄, filtered and concentrated under reduced pressure. The residue was purified by flash chromatography on silica gel (Methanol in Dichloromethane=0-5%) to give 4-[(6-chloro-5-methyl-pyridazin-4-yl)methyl]-3-fluoro-pyridin-2-amine (400 mg, 1.58 mmol). ¹H NMR (400 MHz, DMSO-d₆) δ=8.98 (s, 1H), 7.65 (d, J=5.2 Hz, 1H), 6.30 (t, J=5.2 Hz, 1H), 6.21 (s, 2H), 4.08 (s, 2H), 2.32 (s, 3H). ¹⁹F NMR (376.5 MHz, DMSO-d₆) δ=−144.329 ppm.

Step 6: A mixture of 4-[(6-chloro-5-methyl-pyridazin-4-yl)methyl]-3-fluoro-pyridin-2-amine (360 mg, 1.42 mmol) 4-chloro-2-fluoro-aniline (414.78 mg, 2.85 mmol), Cs₂CO₃ (1.39 g, 4.27 mmol) and Pd₂(dba)₃ (130.47 mg, 142.48 μmol), BINAP (177.43 mg, 284.95 μmol) in dioxane (5 mL) under N₂ was stirred at 110° C. for 2 h. The mixture was filtered and the filtrate was concentrated. The residue was purified by flash chromatography on silica gel (Methanol in Dichloromethane=0-8%) and then purified by flash chromatography on silica gel (Ethyl acetate in Petroleum ether=0-100%) to give 5-[(2-amino-3-fluoro-4-pyridyl)methyl]-N-(4-chloro-2-fluoro-phenyl)-4-methyl-pyridazin-3-amine (105 mg, 290.24 μmol). ¹H NMR (400 MHz, CDCl₃) δ=8.61 (t, J=8.8 Hz, 1H), 8.57 (s, 1H), 7.76 (d, J=5.2 Hz, 1H), 7.18-7.12 (m, 2H), 6.45 (d, J=3.2 Hz, 1H), 6.30 (t, J=5.2 Hz, 1H), 4.65 (s, 2H), 3.96 (s, 2H), 2.23 (s, 3H). ¹⁹F NMR (376.5 MHz, CDCl₃) δ=−129.931 ppm, −144.937 ppm.

Step 7: To a solution of 5-[(2-amino-3-fluoro-4-pyridyl)methyl]-N-(4-chloro-2-fluoro-phenyl)-4-methyl-pyridazin-3-amine (100 mg, 276.41 μmol) and Py (218.64 mg, 2.76 mmol, 223.11 μL) in MeCN (1 mL) and DMA (0.8 mL) was added N-methylsulfamoyl chloride (179.07 mg, 1.38 mmol) under N₂. The mixture was stirred at 25° C. for 5 h. The mixture was concentrated. The residue was purified by prep-HPLC (column: Welch Xtimate C18 150*30 mm*5 μm; mobile phase: [water(NH₃H₂O+NH₄HCO₃)−ACN]; B %: 17%-47%, 7 min) to give N-(4-chloro-2-fluoro-phenyl)-5-[[3-fluoro-2-(methylsulfamoylamino)-4-pyridyl]methyl]-4-methyl-pyridazin-3-amine (46.9 mg, 103.10 μmol). ¹H NMR (400 MHz, CD₃CN) δ=8.48 (s, 1H), 8.30 (br s, 1H), 7.95 (d, J=4.8 Hz, 1H), 7.89 (t, J=8.8 Hz, 1H), 7.28 (dd, J=2.0, 10.8 Hz, 1H), 7.24-7.16 (m, 1H), 6.89-6.61 (m, 2H), 5.86 (br s, 1H), 4.06 (s, 2H), 2.59 (s, 3H), 2.20 (s, 3H). ¹⁹F NMR (376.5 MHz, CD₃CN) δ=−122.747 ppm, −141.304 ppm. LCMS Rₜ=1.833 min in 3 min chromatography, 0-30CD, ESI calcd. for C₁₈H₁₈ClF₂N₆O₂S [M+H]⁺455.1, found 455.2.

### Example 53:4-[[5-(4-chloro-2-fluoro-anilino)-4-methyl-3-pyridyl]methyl]-N-(methylsulfamoyl)pyrimidin-2-amine

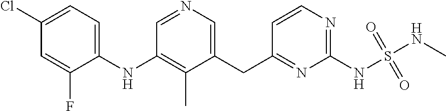 [SMILES: CNS(=O)(=O)Nc1nccc(Cc2cncc(Nc3ccc(Cl)cc3F)c2C)n1]

### Route

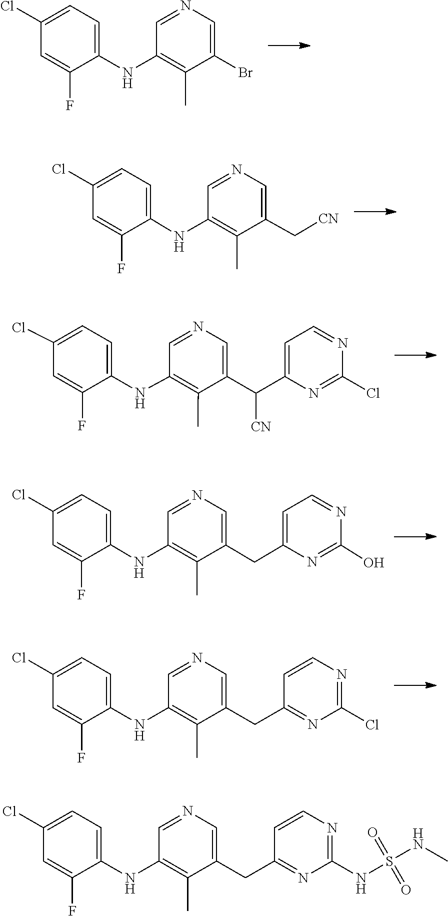 [SMILES: Cc1c(Br)cncc1Nc1ccc(Cl)cc1F ; Cc1c(CC#N)cncc1Nc1ccc(Cl)cc1F ; Cc1c(Nc2ccc(Cl)cc2F)cncc1C(C#N)c1ccnc(Cl)n1 ; Cc1c(Cc2ccnc(O)n2)cncc1Nc1ccc(Cl)cc1F ; Cc1c(Cc2ccnc(Cl)n2)cncc1Nc1ccc(Cl)cc1F ; CNS(=O)(=O)Nc1nccc(Cc2cncc(Nc3ccc(Cl)cc3F)c2C)n1]

Step 1: To a solution of 5-bromo-N-(4-chloro-2-fluoro-phenyl)-4-methyl-pyridin-3-amine (6 g, 19.01 mmol, example 20) and 4-(4,4,5,5-tetramethyl-1,3,2-dioxaborolan-2-yl) isoxazole (6.30 g, 32.32 mmol) in dioxane (60 mL) and H₂O (15 mL) were added Pd(dppf)Cl₂ (695.61 mg, 950.67 umol) and K₃PO₄ (12.11 g, 57.04 mmol). The mixture was stirred at 100° C. for 12 hr. The mixture was poured into sat. NH₄Cl (60 mL) and extracted with EtOAc (20 mL×3). The combined organic layers were washed with brine (60 mL), dried over anhydrous Na₂SO₄, filtered and concentrated under reduced pressure. The crude was purified by flash chromatography on silica gel (Methanol in dichloromethane=0 to 10%) and purified by flash chromatography on silica gel (Ethyl acetate in Petroleum ether=0 to 50%) to give 2-[5-(4-chloro-2-fluoro-anilino)-4-methyl-3-pyridyl]acetonitrile (1.76 g, 6.38 mmol) LCMS Rₜ=3.522 min 7.0 min chromatography, 10-80CD, ESI calcd. for C₁₄H₁₂ClFN [M+H]⁺276.1, found 276.1.

Step 2: To a solution of 2-[5-(4-chloro-2-fluoro-anilino)-4-methyl-3-pyridyl]acetonitrile (1.67 g, 6.06 mmol) in DMF (17 mL) was added NaH (726.79 mg, 18.17 mmol, 60% purity) at 0° C. under N₂. After the mixture was stirred at 0° C. for 30 min, 2,4-dichloropyrimidine (902.38 mg, 6.06 mmol) was added dropwise to the mixture at 0° C. The mixture was stirred at 25° C. for 1.5 hr. The mixture was poured into sat. NH₄Cl (20 mL) and extracted with EtOAc (10 mL×3). The combined organic layers were washed with brine (20 mL), dried over anhydrous Na₂SO₄, filtered and concentrated under reduced pressure. The crude product was triturated from EtOAc/H₂O=1/1 (60 mL) to give 2-[5-(4-chloro-2-fluoro-anilino)-4-methyl-3-pyridyl]-2-(2-chloropyrimidin-4-yl) acetonitrile (2.35 g, 6.05 mmol). LCMS Rₜ=0.67 min 1.5 min chromatography, 5-95 CD, ESI calcd. for C₁₈H₁₃Cl₂FN₅ [M+H]⁺388.1, found 388.1.

Step 3: To a solution of 2-[5-(4-chloro-2-fluoro-anilino)-4-methyl-3-pyridyl]-2-(2-chloropyrimidin-4-yl) acetonitrile (2.35 g, 6.05 mmol) in HCl (28.8 mL) and H₂O (7.20 mL) was added AcOH (9.35 g, 155.64 mmol, 8.90 mL). The mixture was stirred at 100° C. for 4 hr. The mixture was added dropwise into H₂O (30 mL) at 0° C. The mixture was adjusted to pH=7 with saturated NaHCO₃ solution slowly at 0° C. and the aqueous layer was extracted with EtOAc (30 mL×2). The combined organic layer was washed with brine (30 mL), dried over anhydrous Na₂SO₄, filtered and concentrated. The crude was purified by flash chromatography on silica gel (Methanol in Dichloromethane=0 to 5%) to give 4-[[5-(4-chloro-2-fluoro-anilino)-4-methyl-3-pyridyl]methyl]pyrimidin-2-ol (1.2 g, 3.48 mmol). LCMS Rₜ=0.691 min 1.5 min chromatography, 5-95 CD, ESI calcd. for C₁₇H₁₅ClFN₄O [M+H]⁺345.1, found 345.0.

Step 4: To a solution of 4-[[5-(4-chloro-2-fluoro-anilino)-4-methyl-3-pyridyl]methyl]pyrimidin-2-ol (1.2 g, 3.48 mmol) in POCl₃ (12 mL) was added N,N-diethylaniline (519.42 mg, 3.48 mmol, 556.72 mL). The mixture was stirred at 100° C. for 6 hr. The mixture was concentrated. The mixture was added dropwise into H₂O (20 mL) at 25° C. The mixture was adjusted to pH=7 with saturated NaHCO₃ solution slowly at 25° C. and the aqueous layer was extracted with EtOAc (20 mL×2). The combined organic layer was washed with brine (20 mL), dried over anhydrous Na₂SO₄, filtered and concentrated. The crude was purified by flash chromatography on silica gel (Methanol in Dichloromethane=0 to 5%) to give N-(4-chloro-2-fluoro-phenyl)-5-[(2-chloropyrimidin-4-yl)methyl]-4-methyl-pyridin-3-amine (400 mg, 1.10 mmol). LCMS Rₜ=0.719 min 1.5 min chromatography, 5-95 CD, ESI calcd. for C₁₇H₁₄Cl₂FN₄ [M+H]⁺363.1, found 363.0.

Step 5: To a solution of N-(4-chloro-2-fluoro-phenyl)-5-[(2-chloropyrimidin-4-yl)methyl]-4-methyl-pyridin-3-amine (270 mg, 743.36 mmol) and (sulfamoylamino) methane (245.61 mg, 2.23 mmol) in dioxane (2 mL) was added Cs₂CO₃ (363.30 mg, 1.12 mmol), Pd₂(dba)₃ (136.14 mg, 148.67 mmol) and XPhos (141.75 mg, 297.34 mmol). The mixture was stirred at 100° C. for 1 hr. The mixture was concentrated. The mixture was added dropwise into H₂O (5 mL) at 25° C. The mixture was adjusted to pH=7 with saturated NaHCO₃ solution slowly at 25° C. and the aqueous layer was extracted with EtOAc (5 mL×2). The combined organic layer was washed with brine (5 mL), dried over anhydrous Na₂SO₄, filtered and concentrated. The crude was purified by flash chromatography on silica gel (Methanol in Dichloromethane=0 to 5%) and prep-HPLC (column: Phenomenex C18 75×30 mm×3 um; mobile phase: [water(NH₃H₂O+NH₄HCO₃)−ACN]; B %: 20%-50%, 8 min) to give 4-[[5-(4-chloro-2-fluoro-anilino)-4-methyl-3-pyridyl]methyl]-N-(methylsulfamoyl)pyrimidin-2-amine (41.2 mg, 94.30 mmol, 12.69% yield) as a white solid and purified by flash chromatography on silica gel (Methanol in Dichloromethane=0 to 5%) and prep-HPLC (column: Welch Xtimate C18 150×30 mm×5 um; mobile phase: [water(NH₃H₂O+NH₄HCO₃)−ACN]; B %: 46%-76%, 7 min) to give 4-[[5-(4-chloro-2-fluoro-anilino)-4-methyl-3-pyridyl]methyl]-N-methyl-pyrimidin-2-amine (7 mg, 19.56 mmol). ¹H NMR (400 MHz, CDCl₃) δ=8.72-8.60 (m, 1H), 8.46 (d, J=5.2 Hz, 1H), 8.41 (s, 1H), 8.20 (s, 1H), 7.14 (dd, J=2.4, 10.8 Hz, 1H), 7.00 (d, J=8.8 Hz, 1H), 6.86-6.79 (m, 2H), 5.45 (br s, 1H), 4.97-4.87 (m, 1H), 4.14 (s, 2H), 2.59 (d, J=5.2 Hz, 3H), 2.15 (s, 3H). ¹⁹F NMR (376.5 MHz, CDCl₃) δ=−129.747 ppm. LCMS Rₜ=0.708 min in 1.5 min chromatography, 5-95AB, ESI calcd. for C₁₈H₁₉ClFN₆O₂S [M+H]⁺437.1, found 437.0.

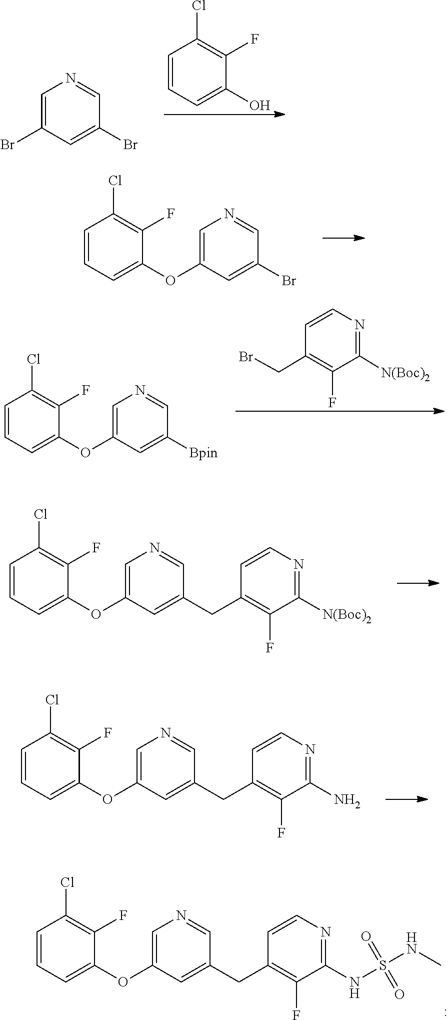

### 4-[[5-(3-chloro-2-fluoro-phenoxy)-3-pyridyl]methyl]-3-fluoro-N-(methylsulfamoyl)pyridin-2-amine

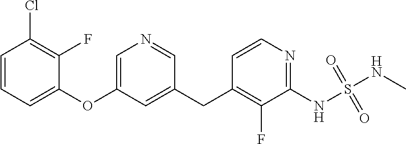 [SMILES: CNS(=O)(=O)Nc1nccc(Cc2cncc(Oc3cccc(Cl)c3F)c2)c1F]

### Route

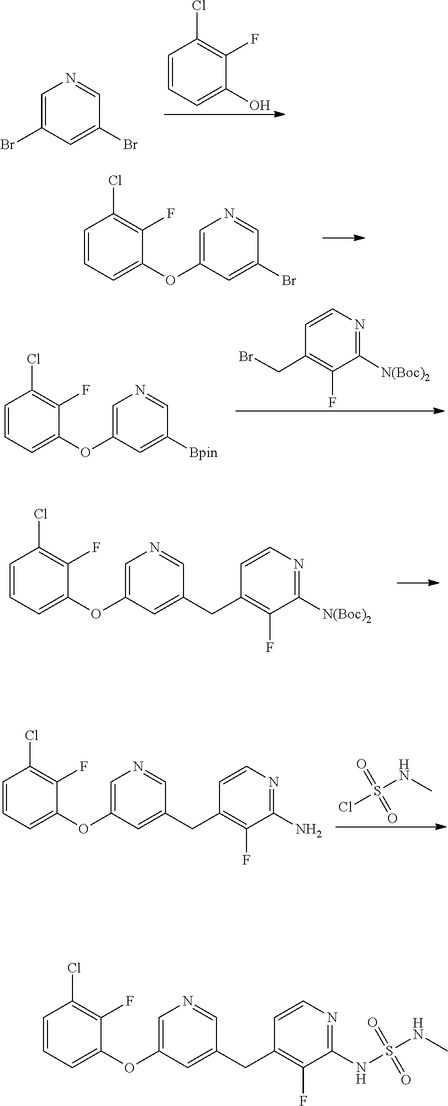

Step 1: To a solution of 3,5-dibromopyridine (5.2 g, 21.95 mmol) in NMP (5 mL) was added Cs₂CO₃ (7.15 g, 21.95 mmol) and 3-chloro-2-fluoro-phenol (3.22 g, 21.95 mmol). The mixture was stirred at 145° C. for 10 hr. The reaction mixture was quenched with water (100 mL). The aqueous layer was extracted with EtOAc (100 mL×3), the combined layers were washed with brine (100 mL), dried over anhydrous Na₂SO₄, filtered and concentrated. The mixture was purified by flash chromatography on silica gel (ethyl acetate in petroleum ether=0-25%) to afford 3-bromo-5-(3-chloro-2-fluoro-phenoxy)pyridine (3.1 g, 10.25 mmol. ¹H NMR (400 MHz, CDCl₃) δ=8.44 (d, J=1.6 Hz, 1H), 8.32 (d, J=2.8 Hz, 1H), 7.40 (t, J=2.4 Hz, 1H), 7.32-7.27 (m, 1H), 7.15-7.07 (m, 1H), 7.07-7.00 (m, 1H). ¹⁹F NMR (376.5 MHz, CDCl₃) δ=−130.807 ppm.

Step 2: To a solution of 3-bromo-5-(3-chloro-2-fluoro-phenoxy)pyridine (2.7 g, 8.92 mmol) in dioxane (2 mL) were added 4,4,5,5-tetramethyl-2-(4,4,5,5-tetramethyl-1,3,2-dioxaborolan-2-yl)-1,3,2-dioxaborolane (3.40 g, 13.39 mmol), Pd(dppf)Cl₂ (653.03 mg, 892.48 μmol) and KOAc (2.63 g, 26.77 mmol). The mixture was stirred at 110° C. for 1.5 hr. The reaction mixture was quenched with water (50 mL). The aqueous layer was extracted with EtOAc (30 mL×3), the combined layers were washed with brine (50 mL), dried over anhydrous Na₂SO₄, filtered and concentrated. The mixture was purified by flash chromatography on silica gel (ethyl acetate in petroleum ether=0-30%) to afford 3-(3-chloro-2-fluoro-phenoxy)-5-(4,4,5,5-tetramethyl-1,3,2-dioxaborolan-2-yl)pyridine (3 g, 8.58 mmol). ¹H NMR (400 MHz, CDCl₃) δ=8.71 (s, 1H), 8.45 (d, J=2.8 Hz, 1H), 7.67-7.59 (m, 1H), 7.25-7.19 (m, 1H), 7.09-7.02 (m, 1H), 6.97-6.91 (m, 1H), 1.33 (s, 12H). ¹⁹F NMR (376.5 MHz, CDCl₃) δ=−131.887 ppm.

Step 3: To a solution of 3-(3-chloro-2-fluoro-phenoxy)-5-(4,4,5,5-tetramethyl-1,3,2-dioxaborolan-2-yl)pyridine (500 mg, 1.43 mmol) in toluene (4 mL) and EtOH (1 mL) were added tert-butyl N-[4-(bromomethyl)-3-fluoro-2-pyridyl]-N-tert-butoxycarbonyl-carbamate (695.54 mg, 1.72 mmol), Pd(PPh₃)₄ (330.55 mg, 286.05 μmol) and a solution of Na₂CO₃ (454.77 mg, 4.29 mmol) in H₂O (0.3 mL). The mixture was stirred at 80° C. for 12 hr. The mixture was concentrated. The mixture was purified by flash chromatography on silica gel (ethyl acetate in petroleum ether=0-30%) to afford tert-butyl N-tert-butoxycarbonyl-N-[4-[[5-(3-chloro-2-fluoro-phenoxy)-3-pyridyl]methyl]-3-fluoro-2-pyridyl]carbamate (300 mg, 547.47 μmol). LCMS Rₜ=0.811 min in 1.5 min chromatography, 5-95AB, ESI calcd. for C₂₇H₂₉ClF₂N₃O₅ [M+H]⁺548.2, found 548.1.

Step 4: To a solution of tert-butyl N-tert-butoxycarbonyl-N-[4-[[5-(3-chloro-2-fluoro-phenoxy)-3-pyridyl]methyl]-3-fluoro-2-pyridyl]carbamate (300 mg, 547.47 μmol) in MeOH (5 mL) was added HCl/MeOH (4 M, 2.74 mL). The mixture was stirred at 25° C. for 2 hr. The mixture was adjusted to pH=7 with saturated NaHCO₃ solution slowly and the aqueous layer was extracted with DCM (10 mL×3). The combined organic layer was washed with brine (20 mL), dried over anhydrous Na₂SO₄, filtered and concentrated. The mixture was purified by flash chromatography on silica gel (ethyl acetate in petroleum ether=0-30%) to afford 4-[[5-(3-chloro-2-fluoro-phenoxy)-3-pyridyl]methyl]-3-fluoro-pyridin-2-amine (130 mg, 373.84 μmol). ¹H NMR (400 MHz, CDCl₃) δ=8.26 (d, J=17.6 Hz, 2H), 7.76 (s, 1H), 7.26-7.20 (m, 1H), 7.13 (s, 1H), 7.07 (t, J=8.0 Hz, 1H), 6.96 (t, J=7.6 Hz, 1H), 6.41 (t, J=4.4 Hz, 1H), 4.72 (s, 2H), 3.93 (s, 2H). ¹⁹F NMR (376.5 MHz, CDCl₃) δ=−131.380 ppm, −145.676 ppm.

Step 5: To a solution of 4-[[5-(3-chloro-2-fluoro-phenoxy)-3-pyridyl]methyl]-3-fluoro-pyridin-2-amine (100 mg, 287.57 μmol) in MeCN (2 mL) were added N-methylsulfamoyl chloride (186.29 mg, 1.44 mmol) and Py (227.46 mg, 2.88 mmol, 232.11 μL). The mixture was stirred at 25° C. for 2 hr. The reaction solution was concentrated. The residue was purified by Prep-HPLC (column: Boston Prime C18 150*30 mm*5 um; mobile phase: [water (NH₃H₂O+NH₄HCO₃)−ACN]; B %: 25%-55%, 7 min) to give 4-[[5-(3-chloro-2-fluoro-phenoxy)-3-pyridyl]methyl]-3-fluoro-N-(methylsulfamoyl)pyridin-2-amine (14 mg, 31.76 umol). ¹H NMR (400 MHz, DMSO-d₆) δ=10.35 (br s, 1H), 8.35-8.26 (m, 2H), 7.98 (d, J=4.4 Hz, 1H), 7.51-7.40 (m, 2H), 7.32-7.14 (m, 2H), 6.96-6.87 (m, 1H), 4.06 (s, 2H), 2.49 (s, 3H). ¹⁹F NMR (376.5 MHz, DMSO-d₆) δ=−133.580 ppm, −138.975 ppm. LCMS Rₜ=0.808 min in 1.5 min chromatography, 5-95AB, ESI calcd. for C₁₈H₁₆ClF₂N₄O₃S [M+H]⁺441.1, found 441.1.

### Example 55: give N-[[5-[3-fluoro-2-(methylsulfamoylamino)-4-pyridyl]methyl]-4-methyl-3-pyridyl]pyrimidin-2-amine

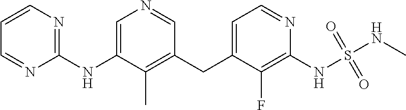 [SMILES: CNS(=O)(=O)Nc1nccc(Cc2cncc(Nc3ncccn3)c2C)c1F]

### Route

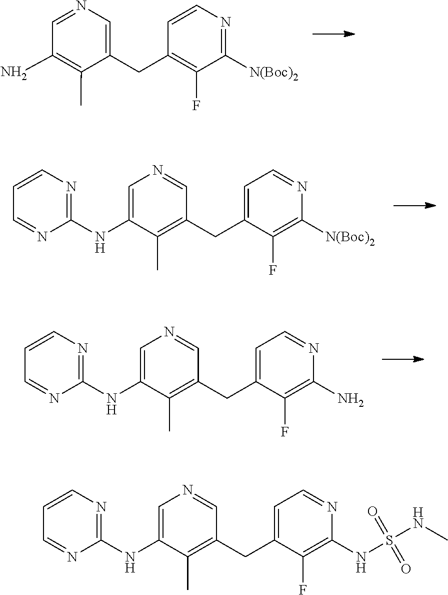

Step 1: To a solution of tert-butyl N-[4-[(5-amino-4-methyl-3-pyridyl)methyl]-3-fluoro-2-pyridyl]-N-tert-butoxycarbonyl-carbamate (250 mg, 578.05 mmol) in dioxane (5 mL) was added 2-chloropyrimidine (66.21 mg, 578.05 mmol), Pd₂(dba)₃ (105.87 mg, 115.61 mmol), Xantphos (33.45 mg, 57.81 mmol) and Cs₂CO₃ (565.02 mg, 1.73 mmol). The mixture was stirred at 80° C. for 3 h. Water (20 mL) was added and the aqueous layer was extracted with EtOAc (50 mL×2). The combined organic layers were washed with brine (50 mL), dried over anhydrous Na₂SO₄, filtered and concentrated. The crude was purified by flash chromatography on silica gel (EtOAc in petroleum ether=0-80%) to give tert-butyl N-tert-butoxycarbonyl-N-[3-fluoro-4-[[4-methyl-5-(pyrimidin-2-ylamino)-3-pyridyl]methyl]-2-pyridyl]carbamate (200 mg, 391.73 mmol). LCMS Rₜ=2.672 min in 7.0 min chromatography, 10-80AB, ESI calcd. for C₂₆H₃₂FN₆O₄ [M+H]⁺511.2, found 511.2.

Step 2: To a solution of tert-butyl N-tert-butoxycarbonyl-N-[3-fluoro-4-[[4-methyl-5-(pyrimidin-2-ylamino)-3-pyridyl]methyl]-2-pyridyl]carbamate (200 mg, 391.73 μmol) in MeOH (2 mL) was added HCl/MeOH (4 M, 1 mL). The mixture was stirred at 25° C. for 2 h. The mixture was concentrated. The residue was purified by prep-HPLC (column: Boston Prime C18 150×30 mm×5 um; mobile phase: [water(NH₃H₂O+NH₄HCO₃)−ACN]; B %: 27%-57%, 7 min) to afford N-[5-[(2-amino-3-fluoro-4-pyridyl)methyl]-4-methyl-3-pyridyl]pyrimidin-2-amine (50 mg, 161.12 μmol). ¹H NMR (400 MHz, CDCl₃) δ=8.94 (br s, 1H), 8.39 (d, J=4.8 Hz, 2H), 8.24 (br s, 1H), 7.71 (d, J=4.8 Hz, 1H), 6.84 (s, 1H), 6.74 (t, J=4.8 Hz, 1H), 6.26 (t, J=4.8 Hz, 1H), 4.62 (br s, 2H), 4.00 (s, 2H), 2.17 (s, 3H). ¹⁹F NMR (376.5 MHz, CDCl₃) δ=−145.380. LCMS Rₜ=0.272 min in 1.5 min chromatography, 5-95AB, ESI calcd. for C₁₆H₁₆FN₆ [M+H]⁺311.1, found 311.0.

Step 3: To a solution of tert-butyl N-tert-butoxycarbonyl-N-[3-fluoro-4-[[4-methyl-5-(pyrimidin-2-ylamino)-3-pyridyl]methyl]-2-pyridyl]carbamate (200 mg, 391.73 μmol) in MeOH (2 mL) was added HCl/MeOH (4 M, 1 mL). The mixture was stirred at 25° C. for 2 h. The mixture was concentrated. The residue was purified by prep-HPLC (column: Boston Prime C18 150×30 mm×5 um; mobile phase: [water(NH₃H₂O+NH₄HCO₃)−ACN]; B %: 27%-57%, 7 min) to afford N-[5-[(2-amino-3-fluoro-4-pyridyl)methyl]-4-methyl-3-pyridyl]pyrimidin-2-amine (50 mg, 161.12 μmol,). ¹H NMR (400 MHz, CDCl₃) δ=8.94 (br s, 1H), 8.39 (d, J=4.8 Hz, 2H), 8.24 (br s, 1H), 7.71 d, J=4.8 Hz, 1H), 6.84 (s, 1H), 6.74 (t, J=4.8 Hz, 1H), 6.26 (t, J=4.8 Hz, 1H), 4.62 (br s, 2H), 4.00 (s, 2H), 2.17 (s, 3H). ¹⁹F NMR (376.5 MHz, CDCl₃) δ=−145.380. LCMS Rₜ=0.272 min in 1.5 min chromatography, 5-95AB, ESI calcd. for C₁₆H₁₆FN₆ [M+H]⁺311.1, found 311.0.

### Example 56: [(4-{[5-(5-chloro-2-fluorophenyl)-4-methylpyridin-3-yl]methyl}-3-fluoropyridin-2-yl)sulfamoyl](methyl)amine

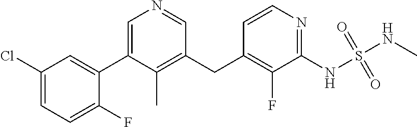 [SMILES: CNS(=O)(=O)Nc1nccc(Cc2cncc(-c3cc(Cl)ccc3F)c2C)c1F]

### Route

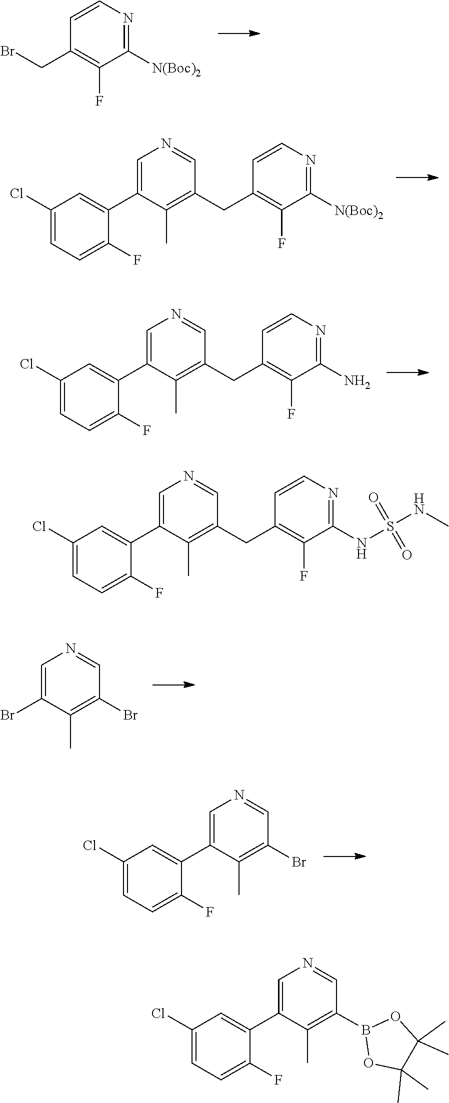

Step 1: A mixture of 3,5-dibromo-4-methylpyridine (5 g, 19.927 mmol), 5-chloro-2-fluorophenylboronic acid (3.47 g, 19.927 mmol), Cs₂CO₃ (12.98 g, 39.854 mmol) and Pd(PPh₃)₄ (2302.70 mg, 1.993 mmol, 0.1 equiv) in dioxane (50 mL) and H₂O (10 mL) was stirred for 2 h at 80° C. under nitrogen atmosphere. Desired product could be detected by LCMS. The resulting mixture was concentrated under reduced pressure. The residue was purified by silica gel column chromatography, eluted with PE/EA (5:1) to afford 3-bromo-5-(5-chloro-2-fluorophenyl)-4-methylpyridine (1.7 g). LCMS: (ESI, m/z): [M+1]⁺=299.7. ¹H NMR (400 MHz, Chloroform-d) δ 8.71 (s, 1H), 8.32 (s, 1H), 7.40 (m, 1H), 7.26-7.22 (m, 1H), 7.14 (t, J=8.9 Hz, 1H), 2.29 (d, J=1.8 Hz, 3H). ¹⁹F NMR (376 MHz, Chloroform-d) 8-116.60.

Step 2: A mixture of 3-bromo-5-(5-chloro-2-fluorophenyl)-4-methylpyridine (200 mg, 0.665 mmol), 4,4,5,5-tetramethyl-2-(tetramethyl-1,3,2-dioxaborolan-2-yl)-1,3,2-dioxaborolane (337.95 mg, 1.330 mmol), AcOK (195.92 mg, 1.995 mmol, 3 equiv) and Pd(dppf)Cl₂ (48.69 mg, 0.067 mmol, 0.1 equiv) in dioxane (5 mL) was stirred for 16 h at 100° C. under nitrogen atmosphere. Desired product could be detected by LCMS. The resulting mixture was concentrated under reduced pressure to afford crude 3-(5-chloro-2-fluorophenyl)-4-methyl-5-(4,4,5,5-tetramethyl-1,3,2-dioxaborolan-2-yl)pyridine. LCMS: (ESI, m/z): [M+1]⁺=348.05

Step 3: A mixture of tert-butyl N-[4-(bromomethyl)-3-fluoropyridin-2-yl]-N-(tert-butoxycarbonyl) carbamate (100 mg, 0.247 mmol), 3-(5-chloro-2-fluorophenyl)-4-methyl-5-(4,4,5,5-tetramethyl-1,3,2-dioxaborolan-2-yl)pyridine (128.66 mg, 0.370 mmol), K₂CO₃ (102.31 mg, 0.741 mmol) and Pd(dppf)Cl₂ (18.06 mg, 0.025 mmol) in dioxane (5 mL) and H₂O (0.5 mL) was stirred for 2 h at 80° C. under nitrogen atmosphere. Desired product could be detected by LCMS. The reaction mixture was diluted with water (20 mL). The resulting mixture was extracted with EA (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (1:1) to afford tert-butyl N-(tert-butoxycarbonyl)-N-(4-{[5-(5-chloro-2-fluorophenyl)-4-methylpyridin-3-yl]methyl}-3-fluoropyridin-2-yl) carbamate (109 mg). LCMS: (ESI, m/z): [M+1]⁺=546.051H NMR (400 MHz, Chloroform-d) δ 8.41 (s, 1H), 8.38 (s, 1H), 8.20 (m, 1H), 7.98 (s, 1H), 7.25-7.23 (m, 1H), 7.12 (m, 1H), 6.94 (m, 1H), 4.13 (s, 2H), 2.08 (m, 3H), 1.41 (s, 18H). ¹⁹F NMR (377 MHz, Chloroform-d) δ −116.85, −130.97.

Step 4: To a stirred solution of tert-butyl N-(tert-butoxycarbonyl)-N-(4-{[5-(5-chloro-2-fluorophenyl)-4-methylpyridin-3-yl]methyl}-3-fluoropyridin-2-yl) carbamate (100 mg, 0.119 mmol) in DCM (1 mL) was added TFA (0.25 mL) dropwise at 0° C. under nitrogen atmosphere. The resulting mixture was stirred for 1 h at room temperature. Desired product could be detected by LCMS. The reaction mixture was basified to pH 10 with sat. NaHCO₃ (aq.). The resulting mixture was extracted with EA (3×20 mL). The combined organic layers were washed with brine (20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 95% gradient in 30 min; detector, UV 254 nm. This resulted in 4-{[5-(5-chloro-2-fluorophenyl)-4-methylpyridin-3-yl]methyl}-3-fluoropyridin-2-amine (23 mg). LCMS: (ESI, m/z): [M+1]⁺=346.05. ¹H NMR (300 MHz, Chloroform-d) δ 8.39 (d, J=17.6 Hz, 2H), 7.72 (d, J=5.4 Hz, 1H), 7.38 (m, 1H), 7.24 (d, J=2.7 Hz, 1H), 7.12 (t, J=8.9 Hz, 1H), 6.30 (t, J=5.2 Hz, 1H), 5.09 (s, 2H), 4.04 (s, 2H), 2.10 (d, J=1.7 Hz, 3H). ¹⁹F NMR (282 MHz, Chloroform-d) δ −116.81, −144.42.

Step 5: To a stirred solution of 4-{[5-(5-chloro-2-fluorophenyl)-4-methylpyridin-3-yl]methyl}-3-fluoropyridin-2-amine (15 mg, 0.043 mmol) and pyridine (17.16 mg, 0.215 mmol, 5 equiv) in DMA (0.5 mL) was added N-methylsulfamoyl chloride (6.74 mg, 0.052 mmol, 1.2 equiv) in DMA (0.3 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in [(4-{[5-(5-chloro-2-fluorophenyl)-4-methylpyridin-3-yl]methyl}-3-fluoropyridin-2-yl)sulfamoyl](methyl)amine (5.4 mg). LCMS: (ESI, m/z): [M+1]⁺=439.0. ¹H NMR (300 MHz, Methanol-d₄) δ 8.41 (s, 1H), 8.31 (s, 1H), 7.98 (d, J=5.2 Hz, 1H), 7.55-7.47 (m, 1H), 7.43-7.38 (m, 1H), 7.32-7.24 (m, 1H), 6.72-6.67 (m, 1H), 4.22 (s, 2H), 2.64 (s, 3H), 2.16 (d, J=1.5 Hz, 3H). ¹⁹F NMR (282 MHz, Methanol-d₄) δ −119.080, −7:42.016.

### Example 57: [(4-{[5-(3-chloro-2-fluorophenyl)-4-methylpyridin-3-yl]methyl}-3-fluoropyridin-2-yl)sulfamoyl](methyl)amine

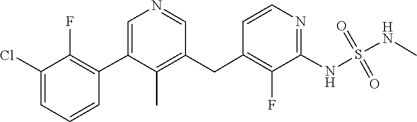 [SMILES: CNS(=O)(=O)Nc1nccc(Cc2cncc(-c3cccc(Cl)c3F)c2C)c1F]

### Route

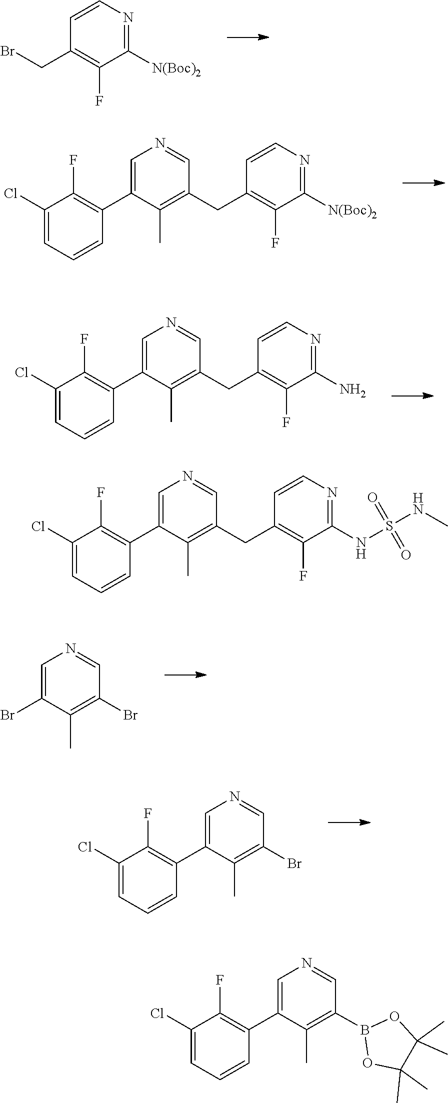

Step 1: A mixture of 3,5-dibromo-4-methylpyridine (5 g, 19.927 mmol), 3-chloro-2-fluorophenylboronic acid (3.47 g, 19.927 mmol), Pd(PPh₃)₄ (2.30 g, 1.993 mmol, 0.1 equiv) and Cs₂CO₃ (12.98 g, 39.854 mmol) in dioxane (45 mL) and H₂O (9 mL) was stirred for 2 h at 80° C. under nitrogen atmosphere. Desired product could be detected by LCMS. The resulting mixture was diluted with water (50 mL). The resulting mixture was extracted with EA (3×50 mL), the combined organic layers were washed with brine (50 mL), dried over anhydrous Na₂SO₄. After filtration, the filtrate was concentrated under reduced pressure. The residue was purified by silica gel column chromatography, eluted with PE/EA (5:1) to afford 3-bromo-5-(3-chloro-2-fluorophenyl)-4-methylpyridine (1.47 g). LCMS: (ESI, m/z): [M+1]⁺=300.05. ¹H NMR (300 MHz, Chloroform-d) δ 8.71 (s, 1H), 8.32 (s, 1H), 7.50 (m, 1H), 7.20 (td, J=7.7, 0.7 Hz, 1H), 7.14 (m, 1H), 2.29 (d, J=1.7 Hz, 3H). ¹⁹F NMR (282 MHz, Chloroform-d) δ −115.65.

Step 2: A mixture of 3-bromo-5-(3-chloro-2-fluorophenyl)-4-methylpyridine (470 mg, 1.564 mmol), 4,4,5,5-tetramethyl-2-(tetramethyl-1,3,2-dioxaborolan-2-yl)-1,3,2-dioxaborolane (595.64 mg, 2.346 mmol, 1.5 equiv), AcOK (391.84 mg, 3.993 mmol, 3 equiv) and Pd(dppf)Cl₂ (97.38 mg, 0.133 mmol, 0.1 equiv) in dioxane (5 mL) was stirred for 16 h at 100° C. under nitrogen atmosphere. Desired product could be detected by LCMS. The reaction mixture was concentrated under reduced pressure to afford crude 3-(3-chloro-2-fluorophenyl)-4-methyl-5-(4,4,5,5-tetramethyl-1,3,2-dioxaborolan-2-yl)pyridine. LCMS: (ESI, m/z): [M+1]⁺=348.05

Step 3: A mixture of tert-butyl N-[4-(bromomethyl)-3-fluoropyridin-2-yl]-N-(tert-butoxycarbonyl) carbamate (135 mg, 0.333 mmol), 3-(3-chloro-2-fluorophenyl)-4-methyl-5-(4,4,5,5-tetramethyl-1,3,2-dioxaborolan-2-yl)pyridine (173.70 mg, 0.500 mmol, 1.5 equiv), Pd(dppf)Cl₂ (24.37 mg, 0.033 mmol, 0.1 equiv) and K₂CO₃ (138.11 mg, 0.999 mmol, 3 equiv) in dioxane (5 mL) and H₂O (0.5 mL) was stirred for 2 h at 80° C. under nitrogen atmosphere. Desired product could be detected by LCMS. The resulting mixture was diluted with water (10 mL) and extracted with EA (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (1:1) to afford tert-butyl N-(tert-butoxycarbonyl)-N-(4-{[5-(3-chloro-2-fluorophenyl)-4-methylpyridin-3-yl]methyl}-3-fluoropyridin-2-yl) carbamate (84 mg). LCMS: (ESI, m/z): [M+1]⁺=546.20. ¹H NMR (400 MHz, Chloroform-d) δ 8.44 (s, 2H), 8.24 (s, 1H), 7.52 (t, J=7.3 Hz, 1H), 7.19 (d, J=24.2 Hz, 2H), 6.96 (s, 1H), 4.18 (s, 2H), 2.14 (s, 3H), 1.42 (s, 18H). ¹⁹F NMR (377 MHz, Chloroform-d) δ −115.66, −130.71.

Step 4: To a stirred solution of tert-butyl N-(tert-butoxycarbonyl)-N-(4-{[5-(3-chloro-2-fluorophenyl)-4-methylpyridin-3-yl]methyl}-3-fluoropyridin-2-yl) carbamate (74 mg, 0.136 mmol) in DCM (1 mL) was added TFA (0.25 mL) dropwise at 0° C. under nitrogen atmosphere. The resulting mixture was stirred for additional 1 h at room temperature. Desired product could be detected by LCMS. The mixture was basified to pH 10 with sat. NaHCO₃ (aq.). The resulting mixture was extracted with EA (3×20 mL). The combined organic layers were washed with brine (20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 95% gradient in 30 min; detector, UV 254 nm. This resulted in 4-{[5-(3-chloro-2-fluorophenyl)-4-methylpyridin-3-yl]methyl}-3-fluoropyridin-2-amine (40 mg). LCMS: (ESI, m/z): [M+1]⁺=346.10. ¹H NMR (300 MHz, Chloroform-d) δ 8.41 (m, 2H), 7.76 (d, J=5.3 Hz, 1H), 7.56-7.46 (m, 1H), 7.25-7.14 (m, 2H), 6.33 (t, J=5.1 Hz, 1H), 4.81 (s, 2H), 4.06 (s, 2H), 2.12 (d, J=1.7 Hz, 3H). ¹⁹F NMR (282 MHz, Chloroform-d) δ −115.85, −144.87.

Step 5: To a stirred solution of 4-{[5-(3-chloro-2-fluorophenyl)-4-methylpyridin-3-yl]methyl}-3-fluoropyridin-2-amine (25 mg, 0.072 mmol) in DMA (1 mL) and pyridine (28.59 mg, 0.360 mmol, 5 equiv) was added N-methylsulfamoyl chloride (11.24 mg, 0.086 mmol, 1.2 equiv) in DMA (0.2 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, MeCN in water (10 mmol/L NH₄HCO₃), 5% to 60% gradient in 30 min; detector, UV 254 nm. This resulted in [(4-{[5-(3-chloro-2-fluorophenyl)-4-methylpyridin-3-yl]methyl}-3-fluoropyridin-2-yl)sulfamoyl](methyl)amine (30.8 mg). LCMS: (ESI, m/z): [M+1]⁺=438.90. ¹H NMR (300 MHz, Methanol-d₄) δ 8.40 (s, 1H), 8.30 (s, 1H), 8.00 (d, J=5.1 Hz, 1H), 7.65-7.54 (m, 1H), 7.35-7.24 (m, 2H), 6.78-6.74 (m, 1H), 4.22 (s, 2H), 2.63 (s, 3H), 2.13 (d, J=1.5 Hz, 3H). ¹⁹F NMR (282 MHz, Methanol-d₄) δ −118.63, −142.31.

### Example 58: 3-fluoro-4-[[5-(2-fluoro-4-isopropyl-anilino)-4-methyl-3-pyridyl]methyl]-N-(methylsulfamoyl)pyridin-2-amine

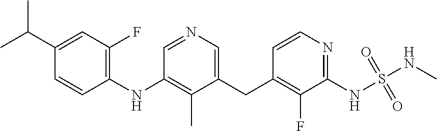 [SMILES: CNS(=O)(=O)Nc1nccc(Cc2cncc(Nc3ccc(C(C)C)cc3F)c2C)c1F]

### Route

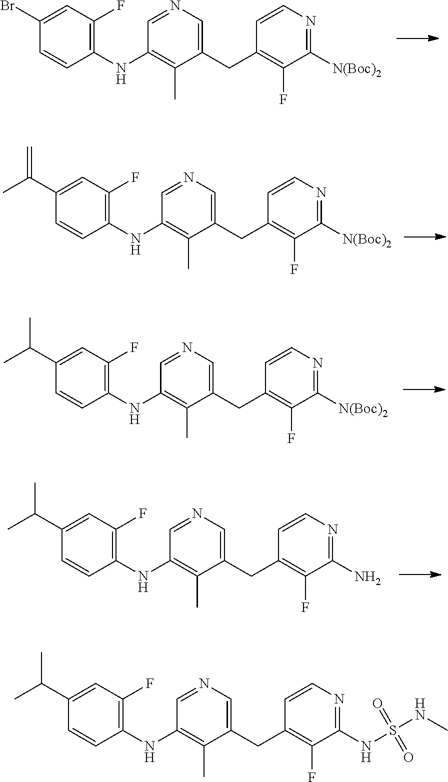

Step 1: To a solution of tert-butyl N-[4-[[5-(4-bromo-2-fluoro-anilino)-4-methyl-3-pyridyl]methyl]-3-fluoro-2-pyridyl]-N-tert-butoxycarbonyl-carbamate (500 mg, 825.81 umol) in dioxane (10 mL) and H₂O (0.5 mL) were added Pd(dppf)Cl₂·CH₂Cl₂ (67.44 mg, 82.58 μmol), K₂CO₃ (228.26 mg, 1.65 mmol) and 2-isopropenyl-4,4,5,5-tetramethyl-1,3,2-dioxaborolane (277.54 mg, 1.65 mmol). The mixture was stirred at 75° C. for 3 h. The mixture was concentrated. The mixture was purified by flash chromatography on silica gel (MeOH in DCM=0-4%) to afford tert-butyl N-tert-butoxycarbonyl-N-[3-fluoro-4-[[5-(2-fluoro-4-isopropenyl-anilino)-4-methyl-3-pyridyl]methyl]-2-pyridyl]carbamate (460 mg, 811.81 μmol). LCMS Rₜ=0.522 min in 1 min chromatography, 5-95 AB, ESI calcd. for C₃₁H₃₇F₂N₄O₄ [M−C₅H₈O₂+H]⁺467.3, found 467.2.

Step 2: To a solution of tert-butyl N-tert-butoxycarbonyl-N-[3-fluoro-4-[[5-(2-fluoro-4-isopropenyl-anilino)-4-methyl-3-pyridyl]methyl]-2-pyridyl]carbamate (450 mg, 794.16 umol) in MeOH (2 mL) was added Pd/C (500 mg, 10% purity). The mixture was stirred at 25° C. for 8 hr under H₂ (50 Psi). The reaction mixture was filtered through a pad of Celite and washed with EtOAc (10 mL×3). The filtrate was concentrated to give tert-butyl N-tert-butoxycarbonyl-N-[3-fluoro-4-[[5-(2-fluoro-4-isopropyl-anilino)-4-methyl-3-pyridyl]methyl]-2-pyridyl]carbamate (451.6 mg, 794.16 μmol) LCMS Rt=5.257 min in 7 min chromatography, 10-80 CD, ESI calcd. for C₃₁H₃₉F₂N₄O₄ [M+H]⁺569.3, found 569.4.

Step 3: To a solution of tert-butyl N-tert-butoxycarbonyl-N-[3-fluoro-4-[[5-(2-fluoro-4-isopropyl-anilino)-4-methyl-3-pyridyl]methyl]-2-pyridyl]carbamate (200 mg, 351.71 μmol) in MeOH (2 mL) was added HCl/MeOH (4 M, 9.70 mL). The mixture was stirred at 20° C. for 3 h. The mixture was concentrated to give 3-fluoro-4-[[5-(2-fluoro-4-isopropyl-anilino)-4-methyl-3-pyridyl]methyl]pyridin-2-amine (180.8 mg, 351.57 μmol, 4HCl). LCMS Rt=0.726 min in 1.5 min chromatography, 5-95AB, ESI calcd. for C₂₁H₂₃F₂N₄ [M+H]⁺369.3, found 369.5.

Step 4: To a solution of 3-fluoro-4-[[5-(2-fluoro-4-isopropyl-anilino)-4-methyl-3-pyridyl]methyl]pyridin-2-amine (150 mg, 291.68 μmol, 4HCl) in MeCN (2 mL) were added Py. (230.72 mg, 2.92 mmol, 235.43 μL) and methylsulfamoyl chloride (188.96 mg, 1.46 mmol) at 25° C. The mixture was stirred at 25° C. for 1 h. The mixture was concentrated. The crude was purified by prep-HPLC (column: Phenomenex C18 75×30 mm×3 μm; mobile phase: [water(NH₃H₂O+NH₄HCO₃)−ACN]; B %: 25%-55%, 8 min) to give 3-fluoro-4-[[5-(2-fluoro-4-isopropyl-anilino)-4-methyl-3-pyridyl]methyl]-N-(methylsulfamoyl)pyridin-2-amine (27.0 mg, 58.50 μmol). ¹H NMR (400 MHz, CDCl₃) δ=8.40 (s, 1H), 8.11 (s, 1H), 7.95 (d, J=5.2 Hz, 1H), 7.04-6.96 (m, 1H), 6.92-6.82 (m, 2H), 6.59 (t, J=5.2 Hz, 1H), 5.47 (br s, 1H), 5.32 (s, 1H), 4.04 (s, 2H), 2.91-2.79 (m, 1H), 2.77 (s, 3H), 2.13 (s, 3H), 1.23 (d, J=7.2 Hz, 6H). ¹⁹F NMR (376.5 MHz, CDCl₃) δ=−131.755, −142.925. LCMS Rt=0.779 min in 1.5 min chromatography, 5-95AB, ESI calcd. for C₂₂H₂₆F₂N₅O₂S [M+H]⁺462.2, found 462.2.

### Example 59: [(4-{[5-(5-chloroindol-1-yl)pyridin-3-yl]methyl}-3-fluoropyridin-2-yl)sulfamoyl](methyl)amine

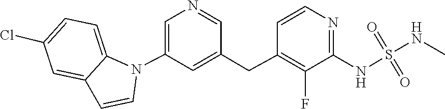 [SMILES: CNS(=O)(=O)Nc1nccc(Cc2cncc(-n3ccc4cc(Cl)ccc43)c2)c1F]

### Route

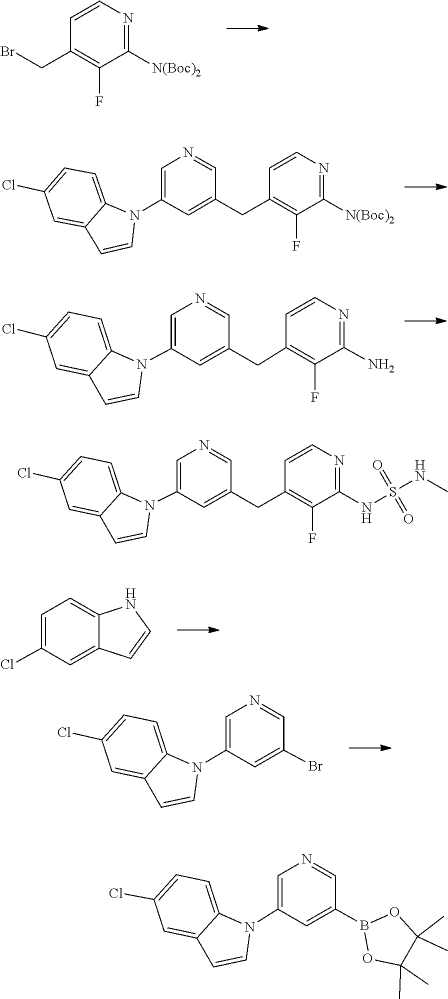

Step 1: A mixture of 5-chloro-1H-indole (2.5 g, 16.492 mmol), 3-bromo-5-iodopyridine (5.15 g, 18.141 mmol), Cs₂CO₃ (10.75 g, 32.984 mmol) and CuI (314.09 mg, 1.649 mmol, 0.1 equiv) in DMF (25 mL) was stirred for 2 h at 100° C. under nitrogen atmosphere. Desired product could be detected by LCMS. The reaction mixture was diluted with water (30 mL). The resulting mixture was extracted with EA (3×30 mL). The combined organic layers were washed with brine (30 mL), dried over anhydrous Na₂SO₄. After filtration, the filtrate was concentrated under reduced pressure. The residue was purified by silica gel column chromatography, eluted with PE/EA (5:1) to afford 1-(5-bromopyridin-3-yl)-5-chloroindole (2.37 g). LCMS: (ESI, m/z): [M+1]⁺=306.90. ¹H NMR (300 MHz, Chloroform-d) δ 8.72 (M, 2H), 7.99 (M, 1H), 7.66 (d, J=2.0 Hz, 1H), 7.43 (d, J=8.8 Hz, 1H), 7.32 (d, J=3.3 Hz, 1H), 7.23 (dd, J=8.8, 2.1 Hz, 1H), 6.70 (dd, J=3.4, 0.9 Hz, 1H).

Step 2: A mixture of 1-(5-bromopyridin-3-yl)-5-chloroindole (400 mg, 1.300 mmol), 4,4,5,5-tetramethyl-2-(tetramethyl-1,3,2-dioxaborolan-2-yl)-1,3,2-dioxaborolane (396.29 mg, 1.560 mmol, 1.2 equiv), AcOK (255.26 mg, 2.600 mmol) and Pd(PPh₃)₂Cl₂ (91.28 mg, 0.130 mmol, 0.1 equiv) in dioxane (5 mL) was stirred for 16 h at 100° C. under nitrogen atmosphere. Desired product could be detected by LCMS. The resulting mixture was concentrated under reduced pressure to afford crude 5-chloro-1-(5-(4,4,5,5-tetramethyl-1,3,2-dioxaborolan-2-yl)pyridin-3-yl)-1H-indole. LCMS: (ESI, m/z): [M+1]⁺=355.05

Step 3: A mixture of tert-butyl N-[4-(bromomethyl)-3-fluoropyridin-2-yl]-N-(tert-butoxycarbonyl) carbamate (175 mg, 0.432 mmol), 5-chloro-1-[5-(4,4,5,5-tetramethyl-1,3,2-dioxaborolan-2-yl)pyridin-3-yl]indole (229.71 mg, 0.648 mmol, 1.5 equiv), Pd(dppf)Cl₂ (31.60 mg, 0.043 mmol, 0.1 equiv) and K₂CO₃ (179.04 mg, 1.296 mmol, 3 equiv) in dioxane (5 mL) and H₂O (0.5 mL) was stirred for 2 h at 80° C. under nitrogen atmosphere. Desired product could be detected by LCMS. The reaction mixture was diluted with water (10 mL). The resulting mixture was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (0.1% FA), 5% to 70% gradient in 30 min; detector, UV 254 nm. This resulted in tert-butyl N-(tert-butoxycarbonyl)-N-(4-{[5-(5-chloroindol-1-yl)pyridin-3-yl]methyl}-3-fluoropyridin-2-yl) carbamate (55 mg). LCMS: (ESI, m/z): [M+1]⁺=553.15. ¹H NMR (400 MHz, Chloroform-d) δ 8.73 (d, J=2.3 Hz, 1H), 8.53 (d, J=2.0 Hz, 1H), 8.28 (d, J=4.9 Hz, 1H), 7.65 (t, J=1.9 Hz, 2H), 7.36 (d, J=8.8 Hz, 1H), 7.30 (d, J=3.3 Hz, 1H), 7.23-7.15 (m, 2H), 6.67 (dd, J=3.3, 0.8 Hz, 1H), 4.18 (s, 2H), 1.37 (s, 18H). ¹⁹F NMR (376 MHz, Chloroform-d) δ −130.91.

Step 4: To a stirred solution of tert-butyl N-(tert-butoxycarbonyl)-N-(4-{[5-(5-chloroindol-1-yl)pyridin-3-yl]methyl}-3-fluoropyridin-2-yl) carbamate (50 mg, 0.090 mmol) in DCM (1 mL) was added TFA (0.25 mL) dropwise at 0° C. under nitrogen atmosphere. The resulting mixture was stirred for 1 h at room temperature. Desired product could be detected by LCMS. The reaction mixture was basified to pH 10 with sat. NaHCO₃ (aq.). The resulting mixture was extracted with EA (3×5 mL). The combined organic layers were washed with brine (5 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 95% gradient in 30 min; detector, UV 254 nm. This resulted in 4-{[5-(5-chloroindol-1-yl)pyridin-3-yl]methyl}-3-fluoropyridin-2-amine (20 mg). LCMS: (ESI, m/z): [M+1]⁺=353.00. ¹H NMR (300 MHz, Chloroform-d) δ 8.63 (d, J=46.0 Hz, 2H), 7.81 (s, 1H), 7.68-7.58 (m, 2H), 7.35 (d, J=8.8 Hz, 1H), 7.30 (d, J=3.3 Hz, 1H), 7.19 (dd, J=8.8, 2.1 Hz, 1H), 6.66 (d, J=3.2 Hz, 1H), 6.51 (s, 1H), 4.73 (s, 2H), 4.06 (s, 2H). ¹⁹F NMR (282 MHz, Chloroform-d) δ 145.53.

Step 5: To a stirred solution of 4-{[5-(5-chloroindol-1-yl)pyridin-3-yl]methyl}-3-fluoropyridin-2-amine (10 mg, 0.028 mmol) in DMA (0.5 mL) were added pyridine (11.21 mg, 0.140 mmol, 5 equiv) and N-methylsulfamoyl chloride (4.41 mg, 0.034 mmol, 1.2 equiv) in DMA (0.2 mL) dropwise at 0° C. under nitrogen atmosphere. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in water (10 mmol/L NH₄HCO₃), 5% to 95% gradient in 30 min; detector, UV 254 nm. This resulted in [(4-{[5-(5-chloroindol-1-yl)pyridin-3-yl]methyl}-3-fluoropyridin-2-yl)sulfamoyl](methyl)amine (11.3 mg). LCMS: (ESI, m/z): [M+1]⁺=445.95. ¹H NMR (400 MHz, Methanol-d₄) δ 8.67 (d, J=2.4 Hz, 1H), 8.50 (d, J=1.8 Hz, 1H), 8.02 (d, J=5.1 Hz, 1H), 7.95-7.87 (m, 1H), 7.63 (d, J=2.0 Hz, 1H), 7.54 (d, J=3.3 Hz, 1H), 7.42 (d, J=8.8 Hz, 1H), 7.20-7.18 (m, 1H), 7.01-6.99 (m, 1H), 6.70 (d, J=3.3 Hz, 1H), 4.21 (s, 2H), 2.61 (s, 3H). ¹⁹F NMR (376 MHz, Methanol-d₄) δ −142.63.

### Example 60: 4-[[5-[(5-ethynyl-3-fluoro-2-pyridyl)amino]-4-methyl-3-pyridyl]methyl]-3-fluoro-N-(methylsulfamoyl)pyridin-2-amine

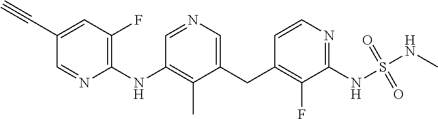 [SMILES: C#Cc1cnc(Nc2cncc(Cc3ccnc(NS(=O)(=O)NC)c3F)c2C)c(F)c1]

### Route

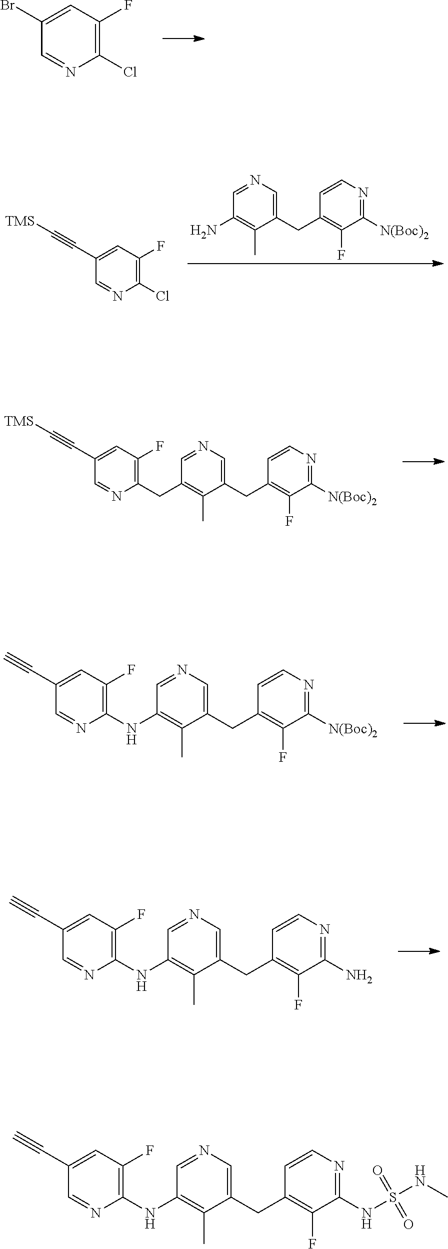

Step 1: To a solution of 5-bromo-2-chloro-3-fluoro-pyridine (2 g, 9.50 mmol) in MeCN (20 mL) were added ethynyl(trimethyl) silane (1.03 g, 10.45 mmol, 1.45 mL), Pd(PPh₃)₂Cl₂ (333.55 mg, 475.21 mmol), CuI (181.01 mg, 950.43 mmol) and TEA (4.81 g, 47.52 mmol, 6.61 mL). The mixture was stirred at 120° C. for 1 hr. Water (80 mL) was added and the mixture were extracted with EtOAc (50 ml×2). The organic layer was dried over anhydrous Na₂SO₄, filtered and concentrated. The crude was purified by flash column chromatography on silica gel (EtOAc in petroleum ether=0-1%) to give 2-(6-chloro-5-fluoro-3-pyridyl) ethynyl-trimethyl-silane (1.1 g, 3.86 mmol, 40.74% yield). LCMS Rt=1.055 min in 1.5 min chromatography, 5-95AB, ESI calcd. for C₁₀H₁₂ClFNSi [M+H]⁺228.0, found 227.9.

Step 2: To a solution of 2-(6-chloro-5-fluoro-3-pyridyl) ethynyl-trimethyl-silane (800 mg, 2.81 mmol, 80% purity) in 1,4-dioxane (14 mL) were added tert-butyl N-[4-[(5-amino-4-methyl-3-pyridyl)methyl]-3-fluoro-2-pyridyl]-N-tert-butoxycarbonyl-carbamate (972.32 mg, 2.25 mmol), Pd(OAc)₂ (63.09 mg, 281.03 mmol), Xantphos (325.21 mg, 562.05 mmol) and Cs₂CO₃ (1.83 g, 5.62 mmol). The mixture was stirred at 80° C. for 4 hr. Water (30 mL) was added and the mixture were extracted with EtOAc (20 mL×2). The organic layer was dried over anhydrous Na₂SO₄, filtered and concentrated. The crude was purified by flash column chromatography on silica gel (EtOAc in petroleum ether=0-65%) to give tert-butyl N-tert-butoxycarbonyl-N-[3-fluoro-4-[[5-[[3-fluoro-5-(2-trimethylsilylethynyl)-2-pyridyl]amino]-4-methyl-3-pyridyl]methyl]-2-pyridyl]carbamate (1 g, 1.60 mmol). ¹H NMR (400 MHz, CDCl₃) δ=8.94 (s, 1H), 8.22-8.18 (m, 3H), 8.07-8.04 (m, 1H), 6.89 (t, J=4.8 Hz, 2H), 4.11 (s, 2H), 2.11 (s, 3H), 1.63 (s, 9H), 1.41 (s, 9H), 0.24 (s, 9H). ¹⁹F NMR (376.5 MHz, CDCl₃) δ=−131.204, −139.763.

Step 3: To a solution of tert-butyl N-tert-butoxycarbonyl-N-[3-fluoro-4-[[5-[[3-fluoro-5-(2-trimethylsilylethynyl)-2-pyridyl]amino]-4-methyl-3-pyridyl]methyl]-2-pyridyl]carbamate (500 mg, 801.59 mmol) in THF (2 mL) was added TBAF (1 M in THF, 8.02 mL). The mixture was stirred at 25° C. for 1 hr. Water (20 mL) was added and the mixture were extracted with EtOAc (20 mL×2). The organic layer was dried over anhydrous Na₂SO₄, filtered and concentrated. The crude was purified by flash column chromatography on silica gel (MeOH in DCM=0-5%) to give tert-butyl N-tert-butoxycarbonyl-N-[4-[[5-[(5-ethynyl-3-fluoro-2-pyridyl)amino]-4-methyl-3-pyridyl]methyl]-3-fluoro-2-pyridyl]carbamate (140 mg, 253.81 mmol). LCMS Rt=0.840 min in 1.5 min chromatography, 5-95AB, ESI calcd. for C₂₉H₃₂F₂N₅O₄ [M+H]⁺552.2, found 552.3.

Step 4: To a solution of tert-butyl N-tert-butoxycarbonyl-N-[4-[[5-[(5-ethynyl-3-fluoro-2-pyridyl)amino]-4-methyl-3-pyridyl]methyl]-3-fluoro-2-pyridyl]carbamate (120 mg, 217.56 mmol) and 2,6-LUTIDINE (46.62 mg, 435.11 mmol, 50.68 mL) in DCM (3 mL) at 0° C. was added TBDMS-OTf (86.26 mg, 326.33 mmol, 75.01 mL). The mixture was stirred at 0° C. for 2 hr. Water (30 mL) was added and the mixture were extracted with EtOAc (20 mL×2). The organic layer was dried over anhydrous Na₂SO₄, filtered and concentrated. The crude was purified by flash column chromatography on silica gel (MeOH in DCM=0-8%) to give 4-[[5-[(5-ethynyl-3-fluoro-2-pyridyl)amino]-4-methyl-3-pyridyl]methyl]-3-fluoro-pyridin-2-amine (76.44 mg, 217.56 mmol). ¹H NMR (400 MHz, CD₃CN) 8=9.28 (s, 1H), 8.24 (s, 1H), 8.12 (d, J=1.6 Hz, 1H), 7.80 (s, 1H), 7.70 (d, J=1.6 Hz, 1H), 7.61 (d, J=6.4 Hz, 1H), 7.20 (s, 2H), 6.64-6.61 (m, 1H), 4.33 (s, 2H), 3.56 (s, 1H), 3.27 (s, 3H). ¹⁹F NMR (376.5 MHz, CD₃CN) δ=−79.407, −136.812.

Step 5: To a solution of 4-[[5-[(5-ethynyl-3-fluoro-2-pyridyl)amino]-4-methyl-3-pyridyl]methyl]-3-fluoro-pyridin-2-amine (66 mg, 187.85 mmol) in MeCN (8 mL) were added N-methylsulfamoyl chloride (121.69 mg, 939.23 mmol) and Py (148.59 mg, 1.88 mmol, 151.62 mL). The mixture was stirred at 0° C. for 1 hr. The mixture was concentrated and purified by Pre-HPLC (column: Phenomenex C 18 80×40 mm×3 mm; mobile phase: [water (0.05% NH₃H₂O 10 mM NH₄HCO₃)−ACN]; B %: 25%-55%, 7 min) to give 4-[[5-[(5-ethynyl-3-fluoro-2-pyridyl)amino]-4-methyl-3-pyridyl]methyl]-3-fluoro-N-(methylsulfamoyl) py ridin-2-amine (26.5 mg, 59.62 mmol). ¹H NMR (400 MHz, CDCl₃) δ=8.94 (brs, 1H), 8.26 (brs, 1H), 8.08 (d, J=1.6 Hz, 1H), 7.94 (d, J=4.8 Hz, 1H), 7.42-7.38 (m, 1H), 6.59 (t, J=5.2 Hz, 1H), 6.39 (s, 1H), 5.48 (s, 1H), 4.07 (s, 2H), 3.12 (s, 1H), 2.77 (d, J=4.8 Hz, 3H), 2.15 (s, 3H). ¹⁹F NMR (376.5 MHz, CDCl₃) δ=−139.514, −142.816. LCMS Rₜ=0.719 min in 1.5 min chromatography, 5-95AB, ESI calcd. for C₂₀H₁₉F₂N₆O₂S [M+H]⁺445.1, found 445.1.

### Example 61: [5-[[3-fluoro-2-(methylsulfamoylamino)-4-pyridyl]methyl]-4-methyl-3-pyridyl] N,N-dimethylcarbamate

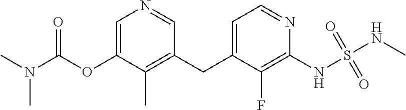

### Route

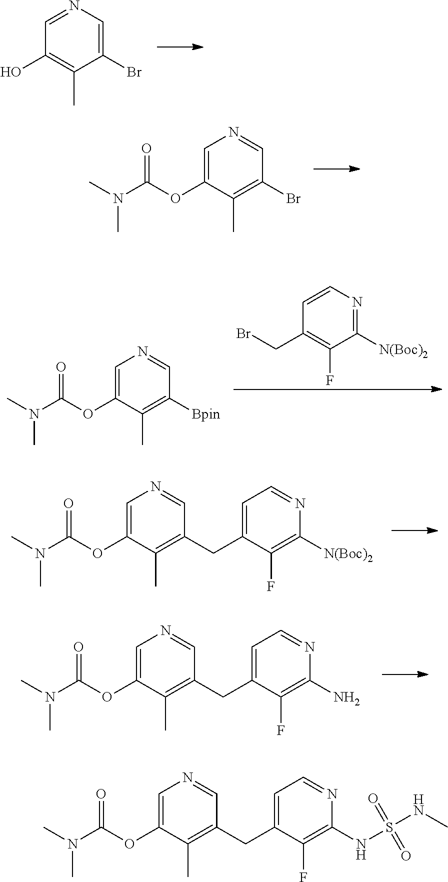

Step 1: To a solution of 5-bromo-4-methyl-pyridin-3-ol (1 g, 5.32 mmol) in DMF (10 mL) was added NaH (233.99 mg, 5.85 mmol, 60% purity) at 0° C. under N₂. After the mixture was stirred at 0° C. for 30 min, N,N-dimethylcarbamoyl chloride (629.14 mg, 5.85 mmol, 537.73 μL) was added dropwise to the above mixture at 0° C. The mixture was stirred at 25° C. for 1.5 hr. The mixture was poured into sat. NH₄Cl (10 mL) and extracted with EtOAc (10 mL×3).

The combined organic layers were washed with brine (10 mL), dried over anhydrous Na₂SO₄, filtered and concentrated under reduced pressure to give (5-bromo-4-methyl-3-pyridyl) N,N-dimethylcarbamate (1.3 g, 5.02 mmol) LCMS Rₜ=0.782 min in 1.5 min chromatography, 5-95AB, ESI calcd. for C₉H₁₂BrN₂O₂ [M+H]⁺261.00, found 260.8.

Step 2: To a solution of (5-bromo-4-methyl-3-pyridyl) N,N-dimethylcarbamate (1.2 g, 4.63 mmol) and 4,4,5,5-tetramethyl-2-(4,4,5,5-tetramethyl-1,3,2-dioxaborolan-2-yl)-1,3,2-dioxaborolane (2.35 g, 9.26 mmol) in dioxane (12 mL) were added KOAc (1.36 g, 13.89 mmol) and Pd(dppf)Cl₂ (338.89 mg, 463.14 μmol). The mixture was stirred at 80° C. for 12 hr. The mixture was filtered. The filtrate was concentrated under reduced pressure. The crude was purified by flash chromatography on silica gel (Methanol in Dichloromethane=0 to 5%) to give [4-methyl-5-(4,4,5,5-tetramethyl-1,3,2-dioxaborolan-2-yl)-3-pyridyl] N,N-dimethylcarbamate (1.42 g, 4.64 mmol). ¹H NMR (400 MHz, CDCl₃) δ=8.70 (s, 1H), 8.34 (s, 1H), 3.15 (s, 3H), 3.03 (s, 3H), 2.39 (s, 3H), 1.34 (s, 12H).

Step 3: To a solution of [4-methyl-5-(4,4,5,5-tetramethyl-1,3,2-dioxaborolan-2-yl)-3-pyridyl] N,N-dimethylcarbamate (1.2 g, 3.92 mmol) and tert-butyl N-[4-(bromomethyl)-3-fluoro-2-pyridyl]-N-tert-butoxycarbonyl-carbamate (1.27 g, 3.14 mmol) in EtOH (5 mL), H₂O (1 mL) and toluene (10 mL) was added Pd(PPh₃)₄ (226.46 mg, 195.97 μmol) and Na₂CO₃ (1.25 g, 11.76 mmol). The mixture was stirred at 80° C. for 1 hr. The reaction was concentrated. The residue was purified by flash chromatography on silica gel (methanol in dichloromethane=0-2%) to afford [5-[[2-[bis(tert-butoxycarbonyl)amino]-3-fluoro-4-pyridyl]methyl]-4-methyl-3-pyridyl] N,N-dimethylcarbamate (1.2 g, 2.38 mmol). ¹H NMR (400 MHz, CDCl₃) δ=8.34 (s, 1H), 8.22 (s, 1H), 8.17 (d, J=5.2 Hz, 1H), 6.87 (t, J=5.2 Hz, 1H), 4.07 (s, 2H), 3.14 (s, 3H), 3.03 (s, 3H), 2.07 (s, 3H), 1.42 (s, 18H).

Step 4: To a solution of [5-[[2-[bis(tert-butoxycarbonyl)amino]-3-fluoro-4-pyridyl]methyl]-4-methyl-3-pyridyl] N,N-dimethylcarbamate (200 mg, 396.39 μmol) in MeCN (2 mL) was added HCl/MeOH (4 M, 2 mL). The mixture was stirred at 25° C. for 2 hr. The reaction was concentrated to give [5-[(2-amino-3-fluoro-4-pyridyl)methyl]-4-methyl-3-pyridyl] N,N-dimethylcarbamate (120 mg, 352.13 μmol, HCl). LCMS Rₜ=0.565 min in 1.5 min chromatography, 5-95AB, ESI calcd. for C₁₅H₁₈FN₄O₂ [M+H]⁺305.1, found 305.0.

Step 5: To a solution of [5-[(2-amino-3-fluoro-4-pyridyl)methyl]-4-methyl-3-pyridyl] N,N-dimethylcarbamate (100 mg, 328.60 μmol) in MeCN (2 mL) were added Py (259.92 mg, 3.29 mmol, 265.23 μL) and N-methylsulfamoyl chloride (212.88 mg, 1.64 mmol). The mixture was stirred at 25° C. for 1 hr. The reaction was concentrated. The residue was purified by flash chromatography on silica gel (Methanol in Dichloromethane=0-10%) and prep-HPLC (column: Boston Prime C18 150×30 mm×5 um; mobile phase: [water (NH₃H₂O+NH₄HCO₃)−ACN]; B %: 18%-48%, 7 min) to afford [5-[[3-fluoro-2-(methylsulfamoylamino)-4-pyridyl]methyl]-4-methyl-3-pyridyl] N,N-dimethylcarbamate (18.9 mg, 47.56 μmol). ¹H NMR (400 MHz, CDCl₃) δ=8.32 (s, 1H), 8.25 (s, 1H), 7.94 (d, J=4.8 Hz, 1H), 7.41-7.27 (m, 1H), 6.58 (t, J=4.8 Hz, 1H), 5.49 (d, J=5.2 Hz, 1H), 4.03 (s, 2H), 3.15 (s, 3H), 3.03 (s, 3H), 2.77 (d, J=5.2 Hz, 3H), 2.10 (s, 3H). ¹⁹F NMR (376.5 MHz, CDCl₃) δ=−142.788 ppm. LCMS Rₜ=0.655 min in 1.5 min chromatography, 5-95AB, ESI calcd. for C₁₆H₂₁FN₅O₄S [M+H]⁺398.1, found 398.0.

### Example 62: 4-[[5-[(5-chloro-3-fluoro-2-pyridyl)amino]-4-methyl-3-pyridyl]methyl]-3-fluoro-N-(methylsulfamoyl)pyridin-2-amine

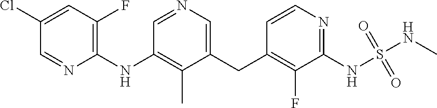 [SMILES: CNS(=O)(=O)Nc1nccc(Cc2cncc(Nc3ncc(Cl)cc3F)c2C)c1F]

### Route

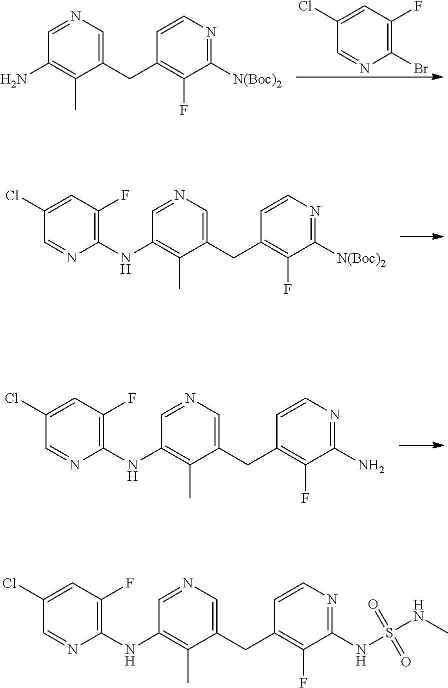

Step 1: To a solution of tert-butyl N-[4-[(5-amino-4-methyl-3-pyridyl)methyl]-3-fluoro-2-pyridyl]-N-tert-butoxycarbonyl-carbamate (400 mg, 924.88 μmol) and 2-bromo-5-chloro-3-fluoro-pyridine (291.94 mg, 1.39 mmol) in dioxane (6 mL) were added Pd(OAc)₂ (10.38 mg, 46.24 μmol), Xantphos (53.52 mg, 92.49 μmol) and Cs₂CO₃ (421.88 mg, 1.29 mmol). The solution was degassed and purged with N₂ for 3 times. The mixture was stirred at 100° C. for 1 hr. The reaction mixture was filtered. The filtrate was diluted with water (10 mL), extracted with Ethyl acetate (5 mL×2). The combined organic layers were washed with brine (10 mL×2), dried over anhydrous Na₂SO₄, concentrated under reduced pressure. The residue was purified by flash chromatography on silica gel (Ethyl acetate in Petroleum ether=0-50%) to give tert-butyl N-tert-butoxycarbonyl-N-[4-[[5-[(5-chloro-3-fluoro-2-pyridyl)amino]-4-methyl-3-pyridyl]methyl]-3-fluoro-2-pyridyl]carbamate (340 mg, 604.97 μmol). ¹H NMR (400 MHz, CDCl₃) δ=8.96 (s, 1H), 8.22 (s, 1H), 8.18 (d, J=4.8 Hz, 1H), 7.93 (d, J=2.0 Hz, 1H), 7.37 (dd, J=2.0, 10.0 Hz, 1H), 6.91-6.87 (t, J=4.8 Hz, 1H), 6.28 (d, J=2.0 Hz, 1H), 4.11 (s, 2H), 2.13 (s, 3H), 1.42 (s, 18H). ¹⁹F NMR (376.5 MHz, CDCl₃) δ=−131.150, −136.294.

Step 2: To a solution of tert-butyl N-[4-[(5-amino-4-methyl-3-pyridyl)methyl]-3-fluoro-2-pyridyl]-N-tert-butoxycarbonyl-carbamate (400 mg, 924.88 μmol) and 2-bromo-5-chloro-3-fluoro-pyridine (291.94 mg, 1.39 mmol) in dioxane (6 mL) were added Pd(OAc)₂ (10.38 mg, 46.24 μmol), Xantphos (53.52 mg, 92.49 μmol) and Cs₂CO₃ (421.88 mg, 1.29 mmol). The solution was degassed and purged with N₂ for 3 times. The mixture was stirred at 100° C. for 1 hr. The reaction mixture was filtered. The filtrate was diluted with water (10 mL), extracted with Ethyl acetate (5 mL×2). The combined organic layers were washed with brine (10 mL×2), dried over anhydrous Na₂SO₄, concentrated under reduced pressure. The residue was purified by flash chromatography on silica gel (Ethyl acetate in Petroleum ether=0-50%) to give tert-butyl N-tert-butoxycarbonyl-N-[4-[[5-[(5-chloro-3-fluoro-2-pyridyl)amino]-4-methyl-3-pyridyl]methyl]-3-fluoro-2-pyridyl]carbamate (340 mg, 604.97 μmol). ¹H NMR (400 MHz, CDCl₃) δ=8.96 (s, 1H), 8.22 (s, 1H), 8.18 (d, J=4.8 Hz, 1H), 7.93 (d, J=2.0 Hz, 1H), 7.37 (dd, J=2.0, 10.0 Hz, 1H), 6.91-6.87 (t, J=4.8 Hz, 1H), 6.28 (d, J=2.0 Hz, 1H), 4.11 (s, 2H), 2.13 (s, 3H), 1.42 (s, 18H). ¹⁹F NMR (376.5 MHz, CDCl₃) δ=−131.150, −136.294.

Step 3: To a solution of 4-[[5-[(5-chloro-3-fluoro-2-pyridyl)amino]-4-methyl-3-pyridyl]methyl]-3-fluoro-pyridin-2-amine (100 mg, 276.41 μmol) and Py (218.64 mg, 2.76 mmol, 223.11 μL) in ACN (1 mL) was added N-methylsulfamoyl chloride (179.07 mg, 1.38 mmol). The mixture was stirred at 25° C. for 2 hr. The solution was concentrated. The mixture was purified by prep-HPLC (column: Phenomenex C18 80×40 mm×3 um; mobile phase: [water(NH₃H₂O+NH₄HCO₃)−ACN]; B %: 25%-55%, 7 min) to give 4-[[5-[(5-chloro-3-fluoro-2-pyridyl)amino]-4-methyl-3-pyridyl]methyl]-3-fluoro-N-(methylsulfamoyl)pyridin-2-amine (73.4 mg, 161.36 μmol). ¹H NMR (400 MHz, CDCl₃) δ=8.99 (s, 1H), 8.24 (s, 1H), 7.97-7.92 (m, 2H), 7.38 (dd, J=10.4, 2.0 Hz, 1H), 6.65-6.55 (m, 1H), 6.31 (s, 1H), 5.48 (s, 1H), 4.07 (s, 2H), 2.76 (d, J=3.2 Hz, 3H), 2.17 (s, 3H) ¹⁹F NMR (376.5 MHz, CDCl₃) δ=−136.203, −142.632. LCMS Rₜ=0.650 min in 1.5 min chromatography, 5-95AB, ESI calcd. for C₁₈H₁₈N₆ClF₂SO₂ [M+H]⁺455.1, found 455.0.

### Example 63: 3-fluoro-4-[[4-methyl-5-(4-methylcyclohexoxy)-3-pyridyl]methyl]-N-(methylsulfamoyl)pyridin-2-amine

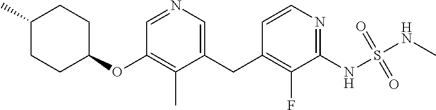

### Route

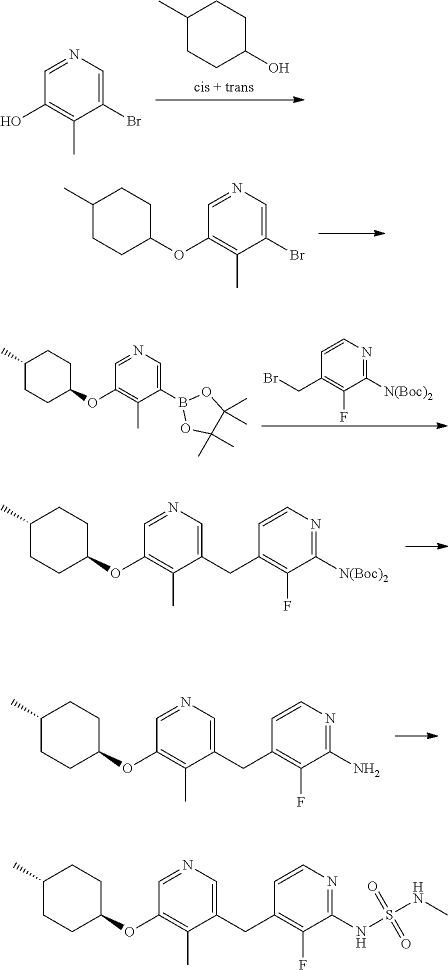

Step 1: To a solution of 5-bromo-4-methyl-pyridin-3-ol (4 g, 21.27 mmol) and 4-methylcyclohexanol (4.86 g, 42.55 mmol, 5.22 mL), PPh₃ (11.16 g, 42.55 mmol) in toluene (60 mL) was added DIAD (8.60 g, 42.55 mmol, 8.27 mL) dropwise under N₂ at 0° C. The mixture was stirred at 80° C. for 1 hr. The mixture was poured into sat. NaHCO₃ (100 mL) in portions at 25° C. and the aqueous layer was extracted with EtOAc (50 mL×3). The combined organic layer was washed with brine (100 mL), dried over anhydrous Na₂SO₄, filtered and concentrated. The residue was purified by flash chromatography on silica gel (Ethyl acetate in Petroleum ether=0-10%) to give 3-bromo-4-methyl-5-(4-methylcyclohexoxy)pyridine (2.3 g, 8.09 mmol, 38.04% yield) as a white solid and 3-bromo-4-methyl-5-(4-methylcyclohexoxy)pyridine (2.8 g, 9.85 mmol, 46.31% yield) was purified by flash chromatography on silica gel (Ethyl acetate in Petroleum ether=0-6%) to give 3-bromo-4-methyl-5-(4-methylcyclohexoxy)pyridine (2.2 g, 7.74 mmol). Intermediate 2: ¹H NMR (400 MHz, CDCl₃) δ=8.27 (s, 1H), 8.09 (s, 1H), 4.31-4.06 (m, 1H), 2.30 (s, 3H), 2.19-2.08 (m, 2H), 1.84-1.74 (m, 2H), 1.53-1.39 (m, 3H), 1.12-0.99 (m, 2H), 0.92 (d, J=6.4 Hz, 3H). Intermediate 2A ¹H NMR (400 MHz, CDCl₃) δ=8.27 (s, 1H), 8.07 (s, 1H), 4.68-4.56 (m, 1H), 2.34 (s, 3H), 2.08-1.97 (m, 2H), 1.64-1.50 (m, 5H), 1.40-1.30 (m, 2H), 0.94 (d, J=6.4 Hz, 3H).

Step 2: A mixture of 3-bromo-4-methyl-5-(4-methylcyclohexoxy)pyridine (800 mg, 2.82 mmol), cyclopentyl(diphenyl) phosphane; dichloropalladium; iron (205.98 mg, 281.50 μmol), 4,4,5,5-tetramethyl-2-(4,4,5,5-tetramethyl-1,3,2-dioxaborolan-2-yl)-1,3,2-dioxaborolane (1.07 g, 4.22 mmol) and KOAc (828.79 mg, 8.45 mmol) in dioxane (8 mL) was stirred at 110° C. stirred for 2 h. The mixture was concentrated. The residue was purified by flash chromatography on silica gel (Ethyl acetate in Petroleum ether=0-100%) to give 4-methyl-3-(4-methylcyclohexoxy)-5-(4,4,5,5-tetramethyl-1,3,2-dioxaborolan-2-yl)pyridine (800 mg, 2.42 mmol). ¹H NMR (400 MHz, CDCl₃) δ=8.44 (s, 1H), 8.20 (s, 1H), 4.22-4.03 (m, 1H), 2.39 (s, 3H), 2.13-2.10 (m, 2H), 1.80-1.71 (m, 2H), 1.52-1.38 (m, 3H), 1.34 (s, 12H), 1.08-0.99 (m, 2H), 0.90 (d, J=6.8 Hz, 3H).

Step 3: A mixture of 4-methyl-3-(4-methylcyclohexoxy)-5-(4,4,5,5-tetramethyl-1,3,2-dioxaborolan-2-yl)pyridine (800.00 mg, 2.42 mmol), tert-butyl N-[4-(bromomethyl)-3-fluoro-2-pyridyl]-N-tert-butoxycarbonyl-carbamate (880.85 mg, 2.17 mmol), Na₂CO₃ (767.91 mg, 7.25 mmol), Pd(PPh₃)₄ (279.07 mg, 241.50 μmol) in toluene (6.4 mL), EtOH (1.6 mL) and H₂O (0.4 mL) was stirred at 80° C. for 1 h under N₂. The mixture was concentrated. The residue was purified by flash chromatography on silica gel (Ethyl acetate in Petroleum ether=0-80%) to give tert-butyl N-tert-butoxycarbonyl-N-[3-fluoro-4-[[4-methyl-5-(4-methylcyclohexoxy)-3-pyridyl]methyl]-2-pyridyl]carbamate (500 mg, 944.03 μmol). ¹H NMR (400 MHz, CDCl₃) δ=8.22-8.11 (m, 2H), 8.00 (s, 1H), 6.86 (t, J=4.8 Hz, 1H), 4.23-4.15 (m, 1H), 4.02 (s, 2H), 2.17-2.09 (m, 2H), 2.05 (s, 3H), 1.85-1.73 (m, 2H), 1.48-1.43 (m, 3H), 1.41 (s, 18H), 1.12-1.00 (m, 2H), 0.92 (d, J=6.8 Hz, 3H) ¹⁹F NMR (376.5 MHz, CDCl₃) δ=−131.287.

Step 4: A mixture of tert-butyl N-tert-butoxycarbonyl-N-[3-fluoro-4-[[4-methyl-5-(4-methylcyclohexoxy)-3-pyridyl]methyl]-2-pyridyl]carbamate (400 mg, 755.23 μmol), HCl/MeOH (4 M, 944.03 μL) was stirred at 25° C. for 1 h under N₂. NH₃/MeOH (7 M, 10 mL) was added dropwise at 0° C. The mixture was concentrated. The residue was purified by flash chromatography on silica gel (MeOH in DCM=0-10%) to give 3-fluoro-4-[[4-methyl-5-(4-methylcyclohexoxy)-3-pyridyl]methyl]pyridin-2-amine (170 mg, 516.07 μmol). ¹H NMR (400 MHz, CDCl₃) δ=8.14 (s, 1H), 8.02 (s, 1H), 7.69 (d, J=5.2 Hz, 1H), 6.22 (t, J=5.2 Hz, 1H), 4.59 (s, 2H), 4.26-4.08 (m, 1H), 3.92 (s, 2H), 2.19-2.10 (m, 2H), 2.09 (s, 3H), 1.81-1.76 (m, 2H), 1.52-1.36 (m, 3H), 1.12-0.97 (m, 2H), 0.92 (d, J=6.4 Hz, 3H). ¹⁹F NMR (376.5 MHz, CDCl₃) δ=−145.436.

Step 5: To a solution of 3-fluoro-4-[[4-methyl-5-(4-methylcyclohexoxy)-3-pyridyl]methyl]pyridin-2-amine (150 mg, 455.36 μmol) and Py (180.09 mg, 2.28 mmol, 183.77 μL) in MeCN (1 mL) was added N-methylsulfamoyl chloride (88.50 mg, 683.04 umol) under N₂. The mixture was stirred at 25° C. for 2 h. Then N-methylsulfamoyl chloride (100.30 mg, 774.11 μmol) was added. The mixture was stirred at 25° C. stirred for 4 h. The mixture was concentrated. The residue was purified by prep-HPLC (column: Phenomenex C18 80*40 mm*3 μm; mobile phase: [water(NH₃H₂O+NH₄HCO₃)−ACN]; gradient: 40%-70% B over 7 min) to give 3-fluoro-4-[[4-methyl-5-(4-methylcyclohexoxy)-3-pyridyl]methyl]-N-(methylsulfamoyl)pyridin-2-amine (40 mg, 94.67 μmol). ¹H NMR (400 MHz, CDCl₃) δ=8.17 (s, 1H), 8.01 (s, 1H), 7.93 (d, J=5.2 Hz, 1H), 6.57 (t, J=5.2 Hz, 1H), 5.47 (s, 1H), 4.30-4.13 (m, 1H), 4.00 (s, 2H), 2.77 (s, 3H), 2.18-2.07 (m, 5H), 1.86-1.76 (m, 2H), 1.55-1.40 (m, 3H), 1.15-1.01 (m, 2H), 0.93 (d, J=6.4 Hz, 3H). ¹⁹F NMR (376.5 MHz, CDCl₃) δ=142.687. LCMS Rₜ=0.942 min in 2 min chromatography, 10-80AB, ESI calcd. for C₂₀H₂₈FN₄O₃S [M+H]⁺422.2, found 423.0.

### Example 64: 4-[[1-(4-chloro-2-fluoro-phenyl) pyrrolo[2,3-c]pyridin-4-yl]methyl]-3-fluoro-N-(methylsulfamoyl)pyridin-2-amine

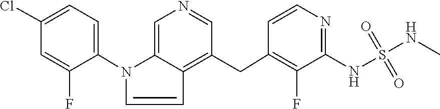 [SMILES: CNS(=O)(=O)Nc1nccc(Cc2cncc3c2ccn3-c2ccc(Cl)cc2F)c1F]

### Route

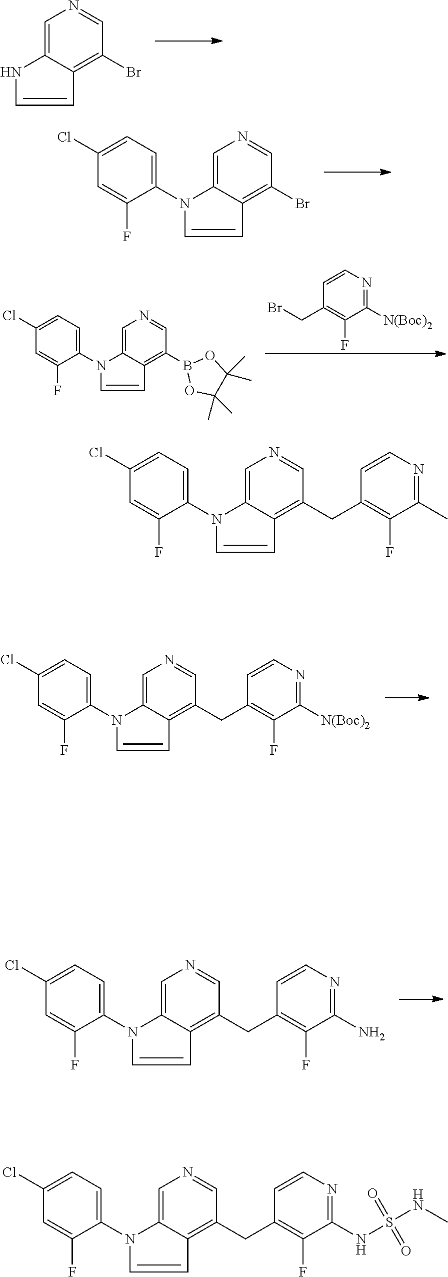

Step 1: To a solution of 4-chloro-2-fluoro-1-iodo-benzene (15.62 g, 60.90 mmol) in dioxane (35 mL) were added 4-bromo-1H-pyrrolo[2,3-c]pyridine (3 g, 15.23 mmol), CuI (1.74 g, 9.14 mmol), K₃PO₄ (25.86 g, 121.81 mmol) and (1S,2S)-cyclohexane-1,2-diamine (869.32 mg, 7.61 mmol, 933.75 μL). The mixture was stirred at 135° C. for 14 h. After cooling to 25° C., the resulting mixture was filtered, and the filter cake was washed with EtOAc (50 mL×4). The filtrate was concentrated. The crude product was purified by flash chromatography on silica gel (EtOAc in Petroleum ether=0-12%) to give 4-bromo-1-(4-chloro-2-fluoro-phenyl) pyrrolo[2,3-c]pyridine (1.2 g, 3.69 mmol). ¹H NMR (400 MHz, CDCl₃) δ=8.91-8.23 (m, 2H), 7.54-7.29 (m, 4H), 6.81 (s, 1H). ¹⁹F NMR (376.5 MHz, CDCl₃) δ=−119.177.

Step 2: To a solution of 4-bromo-1-(4-chloro-2-fluoro-phenyl) pyrrolo[2,3-c]pyridine (1.2 g, 3.69 mmol) in dioxane (12 mL) were added 4,4,5,5-tetramethyl-2-(4,4,5,5-tetramethyl-1,3,2-dioxaborolan-2-yl)-1,3,2-dioxaborolane (1.22 g, 4.79 mmol), KOAc (1.09 g, 11.06 mmol) and Pd(dppf)Cl₂ (269.70 mg, 368.59 mmol). The mixture was stirred at 110° C. for 12 h. 1-(4-chloro-2-fluoro-phenyl)-4-(4,4,5,5-tetramethyl-1,3,2-dioxaborolan-2-yl) pyrrolo[2,3-c]pyridine (1.37 g, 3.68 mmol). LCMS Rₜ=1.791 min 3 min chromatography, 10-80CD, ESI calcd. for C₁₉H₂₀BClFN₂O₂ [M+H]⁺373.1 found 373.2.

Step 3: To a solution of 1-(4-chloro-2-fluoro-phenyl)-4-(4,4,5,5-tetramethyl-1,3,2-dioxaborolan-2-yl) pyrrolo[2,3-c]pyridine (1.37 g, 3.68 mmol) in toluene (1 mL), EtOH (0.2 mL) and H₂O (0.1 mL) were added tert-butyl N-[4-(bromomethyl)-3-fluoro-2-pyridyl]-N-tert-butoxycarbonyl-carbamate (1.94 g, 4.78 mmol), Pd(PPh₃)₄ (424.85 mg, 367.66 μmol) and Na₂CO₃ (779.36 mg, 7.35 mmol). The mixture was stirred at 80° C. for 2 h. Water (30 mL) was added. The mixture was extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-75%) to give tert-butyl N-tert-butoxycarbonyl-N-[4-[[1-(4-chloro-2-fluoro-phenyl) pyrrolo[2,3-c]pyridin-4-yl]methyl]-3-fluoro-2-pyridyl]carbamate (300 mg, 525.38 μmol). ¹H NMR (400 MHz, CDCl₃) δ=8.61 (s, 1H), 8.22 (s, 1H), 8.15 (d, J=4.8 Hz, 1H), 7.71-7.67 (m, 4H), 7.11-7.04 (m, 1H), 6.69-6.59 (m, 1H), 4.33 (s, 2H), 1.38 (s, 18H). ¹⁹F NMR (376.5 MHz, CDCl₃) δ=−119.380, −131.352.

Step 4: To a solution of 1-(4-chloro-2-fluoro-phenyl)-4-(4,4,5,5-tetramethyl-1,3,2-dioxaborolan-2-yl) pyrrolo[2,3-c]pyridine (1.37 g, 3.68 mmol) in toluene (1 mL), EtOH (0.2 mL) and H₂O (0.1 mL) were added tert-butyl N-[4-(bromomethyl)-3-fluoro-2-pyridyl]-N-tert-butoxycarbonyl-carbamate (1.94 g, 4.78 mmol), Pd(PPh₃)₄ (424.85 mg, 367.66 μmol) and Na₂CO₃ (779.36 mg, 7.35 mmol). The mixture was stirred at 80° C. for 2 h. Water (30 mL) was added. The mixture was extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-75%) to give tert-butyl N-tert-butoxycarbonyl-N-[4-[[1-(4-chloro-2-fluoro-phenyl) pyrrolo[2,3-c]pyridin-4-yl]methyl]-3-fluoro-2-pyridyl]carbamate (300 mg, 525.38 μmol). ¹H NMR (400 MHz, CDCl₃) δ=8.61 (s, 1H), 8.22 (s, 1H), 8.15 (d, J=4.8 Hz, 1H), 7.71-7.67 (m, 4H), 7.11-7.04 (m, 1H), 6.69-6.59 (m, 1H), 4.33 (s, 2H), 1.38 (s, 18H). ¹⁹F NMR (376.5 MHz, CDCl₃) δ=−119.380, −131.352.

Step 5: To a solution of 4-[[1-(4-chloro-2-fluoro-phenyl) pyrrolo[2,3-c]pyridin-4-yl]methyl]-3-fluoro-pyridin-2-amine (20 mg, 53.94 μmol) in MeCN (0.8 mL) were added Py (42.67 mg, 539.40 μmol, 43.54 μL) and N-methylsulfamoyl chloride (41.93 mg, 323.64 μmol). The mixture was stirred at 25° C. for 4 h. Then N-methylsulfamoyl chloride (41.93 mg, 323.64 μmol) and Py (42.67 mg, 539.40 μmol, 43.54 μL) were added. The mixture was stirred at 25° C. for 4 h. The mixture was concentrated. The crude product was purified by Prep-HPLC (column: Welch Xtimate C18 150×30 mm×5 mm; mobile phase: [water(NH₃H₂O+NH₄HCO₃)−ACN]; gradient: 35%-65% B over 7 min) to give 4-[[1-(4-chloro-2-fluoro-phenyl) pyrrolo[2,3-c]pyridin-4-yl]methyl]-3-fluoro-N-(methylsulfamoyl)pyridin-2-amine (8.8 mg, 18.97 μmol). ¹H NMR (400 MHz, CD₃OD) δ=8.50 (s, 1H), 8.14 (s, 1H), 7.94 (d, J=5.2 Hz, 1H), 7.72 (d, J=2.0 Hz, 1H), 7.66-7.54 (m, 2H), 7.45 (d, J=8.8 Hz, 1H), 6.91-6.82 (m, 2H), 4.38 (s, 2H), 2.61 (s, 3H). ¹⁹F NMR (376.5 MHz, CD₃OD) δ=−121.355, −142.466. LCMS Rₜ=0.673 min 1.5 min chromatography, 5-95AB, ESI calcd. for C₂₀H₁₇ClF₂N₅O₂S [M+H]⁺464.1, found 464.0.

### Example 65: 4-[[5-[4-(difluoromethyl)-2-fluoro-phenoxy]-4-methyl-3-pyridyl]methyl]-3-fluoro-N-(methylsulfamoyl)pyridin-2-amine

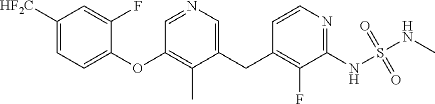

### Route

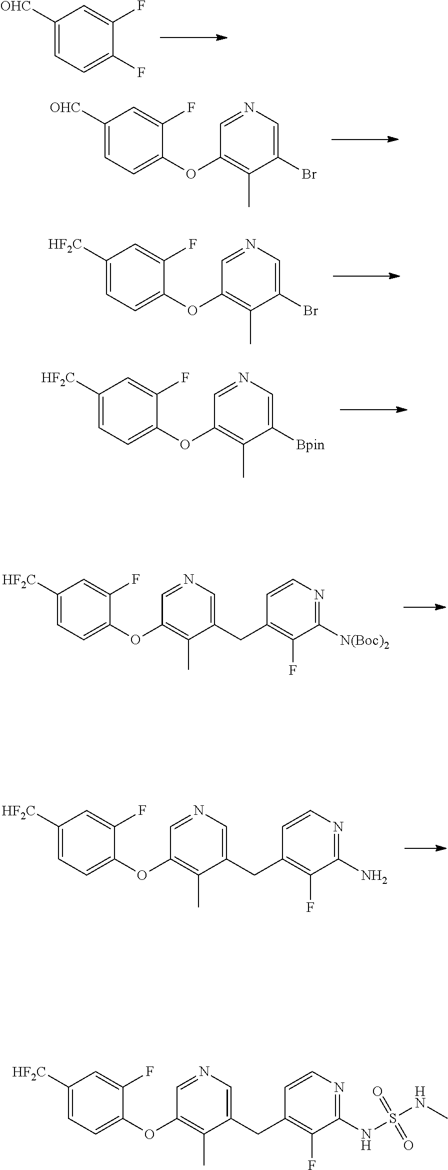

Step 1: A solution of 3,4-difluorobenzaldehyde (3 g, 21.11 mmol, 2.29 mL) in DMF (45 mL) were added 5-bromo-4-methyl-pyridin-3-ol (4.76 g, 25.33 mmol) and K₂CO₃ (5.84 g, 42.22 mmol) was stirred at 25° C. for 30 min. Then mixture was stirred at 120° C. for 17.5 h. The mixture was poured into water (100 mL). The aqueous layer was extracted with EtOAc (100 mL×3). The combined organic phase was washed with H₂O (200 mL×3) and brine (100 mL), dried over anhydrous Na₂SO₄, filtered and concentrated under reduced pressure. The mixture was purified by flash chromatography on silica gel (EtOAc in petroleum ether=0-20%) to afford 4-[(5-bromo-4-methyl-3-pyridyl)oxy]-3-fluoro-benzaldehyde (3.4 g, 10.96 mmol,). ¹H NMR (400 MHz, CDCl₃) δ=9.91 (s, 1H), 8.55 (s, 1H), 8.15 (s, 1H), 7.73 (d, J=10.4 z, 1H), 7.61 (d, J=8.0 Hz, 1H), 6.93 (t, J=8.0 Hz, 1H), 2.36 (s, 3H).

Step 2: To a solution of 4-[(5-bromo-4-methyl-3-pyridyl)oxy]-3-fluoro-benzaldehyde (3.2 g, 10.32 mmol) in DCM (30 mL) were added DAST (3.33 g, 20.64 mmol, 2.73 mL) and EtOH (2 mL) at 0° C. The mixture was stirred at 25° C. for 48 h. The mixture was added to H₂O (20 mL). The aqueous layer was extracted with EtOAc (20 mL×3). The combined organic phase was washed with brine (20 mL), dried over anhydrous Na₂SO₄, filtered and concentrated under reduced pressure. The mixture was purified by flash chromatography on silica gel (EtOAc in petroleum ether=0-15%) to afford 3-bromo-5-[4-(difluoromethyl)-2-fluoro-phenoxy]-4-methyl-pyridine (1.9 g, 5.72 mmol). ¹H NMR (400 MHz, CDCl₃) δ=8.50 (s, 1H), 8.05 (s, 1H), 7.38 (d, J=10.8 Hz, 1H), 7.24 (d, J=8.0 Hz, 1H), 6.96 (t, J=8.0 Hz, 1H), 6.76 (t, J=16.0 Hz, 1H), 2.40 (s, 3H).

Step 3: To a solution of 3-bromo-5-[4-(difluoromethyl)-2-fluoro-phenoxy]-4-methyl-pyridine (1.8 g, 5.42 mmol) in dioxane (20 mL) were added Pd(dppf)Cl₂ (793.14 mg, 1.08 mmol), KOAc (1.60 g, 16.26 mmol) and 4,4,5,5-tetramethyl-2-(4,4,5,5-tetramethyl-1,3,2-dioxaborolan-2-yl)-1,3,2-dioxaborolane (4.13 g, 16.26 mmol). The mixture was stirred at 100° C. for 18 h. The mixture was concentrated to give 3-[4-(difluoromethyl)-2-fluoro-phenoxy]-4-methyl-5-(4,4,5,5-tetramethyl-1,3,2-dioxaborolan-2-yl)pyridine (2.06 g, 5.43 mmol).

Step 4: To a solution of 3-bromo-5-[4-(difluoromethyl)-2-fluoro-phenoxy]-4-methyl-pyridine (1.8 g, 5.42 mmol) in dioxane (20 mL) were added Pd(dppf)Cl₂ (793.14 mg, 1.08 mmol), KOAc (1.60 g, 16.26 mmol) and 4,4,5,5-tetramethyl-2-(4,4,5,5-tetramethyl-1,3,2-dioxaborolan-2-yl)-1,3,2-dioxaborolane (4.13 g, 16.26 mmol). The mixture was stirred at 100° C. for 18 h. The mixture was concentrated to give 3-[4-(difluoromethyl)-2-fluoro-phenoxy]-4-methyl-5-(4,4,5,5-tetramethyl-1,3,2-dioxaborolan-2-yl)pyridine (2.06 g, 5.43 mmol).

Step 5: To a solution of tert-butyl N-tert-butoxycarbonyl-N-[4-[[5-[4-(difluoromethyl)-2-fluoro-phenoxy]-4-methyl-3-pyridyl]methyl]-3-fluoro-2-pyridyl]carbamate (800 mg, 1.39 mmol) in MeOH (2 mL) was added HCl/MeOH (4 M, 38.20 mL). The mixture was stirred at 20° C. for 3 h. The mixture was added NH₃·MeOH to pH=7 and concentrated. The mixture was purified by flash chromatography on silica gel (EtOAc in petroleum ether=0-80%) to afford 4-[[5-[4-(difluoromethyl)-2-fluoro-phenoxy]-4-methyl-3-pyridyl]methyl]-3-fluoro-pyridin-2-amine (300 mg, 795.05 μmol). ¹H NMR (400 MHz, CDCl₃) δ=8.26 (s, 1H), 8.11 (s, 1H), 7.74 (d, J=5.2 Hz, 1H), 7.37 (d, J=10.8 Hz, 1H), 7.22 (d, J=7.6 Hz, 1H), 6.91 (t, J=8.4 Hz, 1H), 6.61 (t, J=56.4 Hz, 1H), 6.27 (t, J=5.2 Hz, 1H), 4.69 (br s, 2H), 4.01 (s, 2H), 2.20 (s, 3H).

Step 6: To a solution of tert-butyl N-tert-butoxycarbonyl-N-[4-[[5-[4-(difluoromethyl)-2-fluoro-phenoxy]-4-methyl-3-pyridyl]methyl]-3-fluoro-2-pyridyl]carbamate (800 mg, 1.39 mmol) in MeOH (2 mL) was added HCl/MeOH (4 M, 38.20 mL). The mixture was stirred at 20° C. for 3 h. The mixture was added NH₃·MeOH to pH=7 and concentrated. The mixture was purified by flash chromatography on silica gel (EtOAc in petroleum ether=0-80%) to afford 4-[[5-[4-(difluoromethyl)-2-fluoro-phenoxy]-4-methyl-3-pyridyl]methyl]-3-fluoro-pyridin-2-amine (300 mg, 795.05 μmol). ¹H NMR (400 MHz, CDCl₃) δ=8.26 (s, 1H), 8.11 (s, 1H), 7.74 (d, J=5.2 Hz, 1H), 7.37 (d, J=10.8 Hz, 1H), 7.22 (d, J=7.6 Hz, 1H), 6.91 (t, J=8.4 Hz, 1H), 6.61 (t, J=56.4 Hz, 1H), 6.27 (t, J=5.2 Hz, 1H), 4.69 (br s, 2H), 4.01 (s, 2H), 2.20 (s, 3H).

### Example 66: 4-[[5-[[5-(difluoromethoxy)-2-pyridyl]amino]-4-methyl-3-pyridyl]methyl]-3-fluoro-N-(methylsulfamoyl)pyridin-2-amine

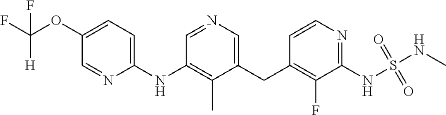 [SMILES: CNS(=O)(=O)Nc1nccc(Cc2cncc(Nc3ccc(OC(F)F)cn3)c2C)c1F]

### Route

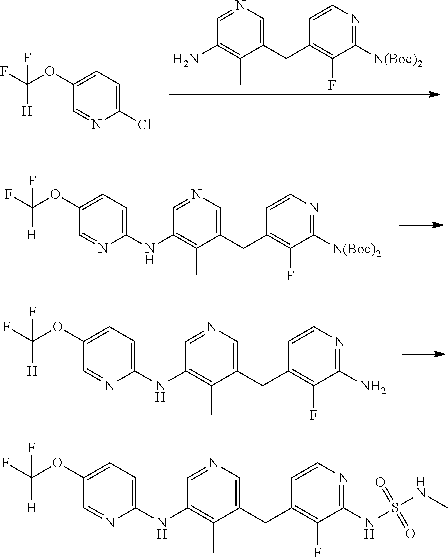

Step 1: To a solution of 2-chloro-5-(difluoromethoxy)pyridine (300 mg, 1.67 mmol) in dioxane (5 mL) were added tert-butyl N-[4-[(5-amino-4-methyl-3-pyridyl)methyl]-3-fluoro-2-pyridyl]-N-tert-butoxycarbonyl-carbamate (722.61 mg, 1.67 mmol), Pd(OAc)₂ (37.51 mg, 167.08 μmol), Xantphos (96.68 mg, 167.08 μmol) and Cs₂CO₃ (1.09 g, 3.34 mmol) under N₂. The mixture was stirred at 100° C. for 2 h. Water (40 mL) was added and the mixture were extracted with EtOAc (40 mL×2). The organic layer was dried over anhydrous Na₂SO₄, filtered and concentrated. The crude was purified by flash column chromatography on silica gel (EtOAc in petroleum ether=0-80%) to give tert-butyl N-tert-butoxycarbonyl-N-[4-[[5-[[5-(difluoromethoxy)-2-pyridyl]amino]-4-methyl-3-pyridyl]methyl]-3-fluoro-2-pyridyl]carbamate (290 mg, 503.84 μmol). ¹H NMR (400 MHz, CDCl₃) δ=8.64 (s, 1H), 8.22 (s, 1H), 8.19 (d, J=4.8 Hz, 1H), 8.06 (d, J=2.4 Hz, 1H), 7.34 (dd, J=2.4, 8.8 Hz, 1H), 8.91 (t, J=4.8 Hz, 1H), 6.63-6.23 (m, 3H), 4.11-4.09 (m, 2H), 2.13 (s, 3H), 1.42 (s, 18H).

Step 2: To a solution of tert-butyl N-tert-butoxycarbonyl-N-[4-[[5-[[5-(difluoromethoxy)-2-pyridyl]amino]-4-methyl-3-pyridyl]methyl]-3-fluoro-2-pyridyl]carbamate (280 mg, 486.47 umol) in MeOH (1 mL) was added HCl/MeOH (4 M, 2 mL). The mixture was stirred at 25° C. for 4 h. The mixture was adjusted to pH=7 by NH₃-MeOH (7 M) and the mixture was concentrated to give 4-[[5-[[5-(difluoromethoxy)-2-pyridyl]amino]-4-methyl-3-pyridyl]methyl]-3-fluoro-pyridin-2-amine (160 mg, 426.27 μmol). ¹H NMR (400 MHz, CDCl₃) δ=8.61 (s, 1H), 8.25 (s, 1H), 8.06 (d, J=2.8 Hz, 1H), 7.72 (d, J=5.2 Hz, 1H), 7.33 (dd, J=2.4, 8.8 Hz, 1H), 6.62-6.23 (m, 4H), 4.68 (br s, 2H), 4.00 (s, 2H), 2.16 (s, 3H).

Step 3: To a solution of 4-[[5-[[5-(difluoromethoxy)-2-pyridyl]amino]-4-methyl-3-pyridyl]methyl]-3-fluoro-pyridin-2-amine (130 mg, 346.35 μmol) in MeCN (2 mL) were added N-methylsulfamoyl chloride (224.37 mg, 1.73 mmol) and py (273.96 mg, 3.46 mmol, 279.55 μL). The mixture was stirred at 0° C. for 2 h. The mixture was concentrated. The crude was purified by prep-HPLC (column: Phenomenex 4-[[5-[[5-(difluoromethoxy)-2-pyridyl]amino]-4-methyl-3-pyridyl]methyl]-3-fluoro-N-(methylsulfamoyl)pyridin-2-amine C18 80×40 mm×3 mm; mobile phase: [water(NH₃H₂O+NH₄HCO₃)−ACN]; gradient: 25%-55% B over 7 min) to give (56.8 mg, 121.25 μmol). ¹H NMR (400 MHz, DMSO-d₆) δ=10.38 (br s, 1H), 8.57 (s, 1H), 8.53 (s, 1H), 8.11 (s, 1H), 7.99 (d, J=4.8 Hz, 1H), 7.93 (d, J=2.8 Hz, 1H), 7.47 (dd, J=2.8, 9.2 Hz, 1H), 7.25-6.86 (m, 2H), 6.82-6.77 (m, 1H), 6.76-6.71 (m, 1H), 4.09 (s, 2H), 2.52-2.51 (s, 3H), 2.08 (s, 3H). ¹⁹F NMR (376.5 MHz, DMSO-d₆) δ=−81.43, −138.18. LCMS Rₜ=0.637 min in 1.5 min chromatography, 5-95AB, ESI calcd. for C₁₉H₂₀N₆F₃O₃S [M+H]⁺469.1, found 469.1.

### Example 67: 4-chloro-N-(4-chloro-2-fluorophenyl)-5-({3-fluoro-2-[(methylsulfamoyl)amino]pyridin-4-yl}methyl)pyridin-3-amine

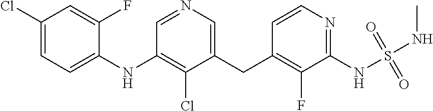 [SMILES: CNS(=O)(=O)Nc1nccc(Cc2cncc(Nc3ccc(Cl)cc3F)c2Cl)c1F]

### Route

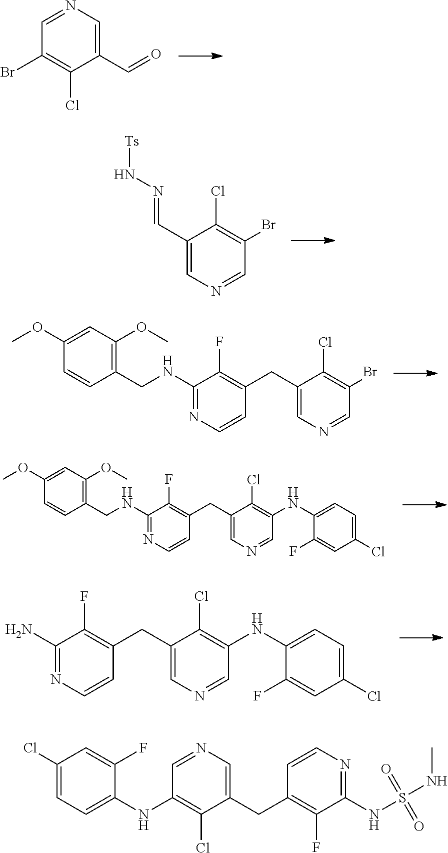 [SMILES: O=Cc1cncc(Br)c1Cl ; Cc1ccc(S(=O)(=O)N/N=C/c2cncc(Br)c2Cl)cc1 ; COc1ccc(CNc2nccc(Cc3cncc(Br)c3Cl)c2F)c(OC)c1 ; COc1ccc(CNc2nccc(Cc3cncc(Nc4ccc(Cl)cc4F)c3Cl)c2F)c(OC)c1 ; Nc1nccc(Cc2cncc(Nc3ccc(Cl)cc3F)c2Cl)c1F ; CNS(=O)(=O)Nc1nccc(Cc2cncc(Nc3ccc(Cl)cc3F)c2Cl)c1F]

Step 1: A mixture of 5-bromo-4-chloropyridine-3-carbaldehyde (500 mg, 2.268 mmol) and 4-toluenesulfonyl hydrazide (464.62 mg, 2.495 mmol, 1.1 equiv) in MeOH (5 mL) was stirred for 2 h at room temperature under nitrogen atmosphere. The precipitated solids were collected by filtration and washed with MeOH (1×3 mL), then further purified by trituration with MeOH (10 mL). This resulted in N′-[(1E)-(5-bromo-4-chloropyridin-3-yl)methylidene]-4-methylbenzenesulfonohydrazide (760 mg). LCMS: (ESI, m/z): [M+1]⁺=388.0. ¹H NMR (400 MHz, DMSO-d₆) δ 12.02 (s, 1H), 8.82 (s, 1H), 8.73 (s, 1H), 8.17 (s, 1H), 7.78 (d, J=8.0 Hz, 2H), 7.43 (d, J=8.0 Hz, 2H), 2.37 (s, 3H).

Step 2: A mixture of N′-[(1E)-(5-bromo-4-chloropyridin-3-yl)methylidene]-4-methylbenzenesulfonohydrazide (600 mg, 1.544 mmol), K₂CO₃ (256.03 mg, 1.853 mmol, 1.2 equiv) and 2-{[(2,4-dimethoxyphenyl)methyl]amino}-3-fluoropyridin-4-ylboronic acid (2362.73 mg, 7.720 mmol, 5 equiv) in dioxane (5 mL) was stirred for 2 h at 110° C. under nitrogen atmosphere. The resulting mixture was concentrated under reduced pressure. The residue was purified by silica gel column chromatography, eluted with PE/EA (2:1) to afford 4-[(5-bromo-4-chloropyridin-3-yl)methyl]-N-[(2,4-dimethoxyphenyl)methyl]-3-fluoropyridin-2-amine (230 mg, 32%) as a white solid.

LCMS: (ESI, m/z): [M+1]⁺=466.0

¹H NMR (400 MHz, Chloroform-d) δ 8.65 (s, 1H), 8.38-8.28 (m, 1H), 7.81 (d, J=5.4 Hz, 1H), 7.32 (dd, =14.9, 8.0 Hz, 1H), 6.53-6.39 (m, 2H), 6.27 (t, J=5.2 Hz, 1H), 4.64 (s, 2H), 4.07 (s, 2H), 3.85 (s, 3H), 3.80 (s, 3H).

Step 3: A mixture of 4-[(5-bromo-4-chloropyridin-3-yl)methyl]-N-[(2,4-dimethoxyphenyl)methyl]-3-fluoropyridin-2-amine (150 mg, 0.321 mmol), 4-chloro-2-fluoroaniline (51.46 mg, 0.353 mmol, 1.1 equiv), Pd₂(dba)₃ (29.43 mg, 0.032 mmol, 0.1 equiv), Cs₂CO₃ (209.42 mg, 0.642 mmol) and XantPhos (18.60 mg, 0.032 mmol, 0.1 equiv) in dioxane (5 mL) was stirred for 16 h at 80° C. under nitrogen atmosphere. The resulting mixture was diluted with water and then extracted with EA (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (2:1) to afford 4-chloro-N-(4-chloro-2-fluorophenyl)-5-[(2-{[(2,4-dimethoxyphenyl)methyl]amino}-3-fluoropyridin-4-yl)methyl]pyridin-3-amine (152 mg). LCMS: (ESI, m/z): [M+1]⁺=531.0. ¹H NMR (300 MHz, Chloroform-d) δ 8.35 (s, 1H), 8.04 (s, 1H), 7.81 (d, J=5.3 Hz, 1H), 7.31-7.21 (m, 1H), 7.24 (d, J=8.6 Hz, 1H), 7.19 (m, 1H), 7.11 (m, 1H), 6.48 (d, J=2.4 Hz, 1H), 6.44 (m, 1H), 6.28 (m, 1H), 5.93 (s, 1H), 5.11 (s, 1H), 4.62 (d, J=5.6 Hz, 2H), 4.04 (s, 2H), 3.85 (s, 3H), 3.80 (s, 3H). ¹⁹F NMR (282 MHz, Chloroform-d) δ −125.26, −147.05.

Step 4: To a stirred solution of 4-chloro-N-(4-chloro-2-fluorophenyl)-5-[(2-{[(2,4-dimethoxyphenyl)methyl]amino}-3-fluoropyridin-4-yl)methyl]pyridin-3-amine (196 mg, 0.369 mmol) in DCM (2 mL) was added TFA (0.5 mL) dropwise at 0° C. under nitrogen atmosphere. The resulting mixture was stirred for 1 h at room temperature. The resulting mixture was concentrated under reduced pressure. The reaction mixture was basified to pH 10 with sat. NaHCO₃ (aq.). The resulting mixture was extracted with EA (3×5 mL). The combined organic layers were washed with brine, dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 95% gradient in 30 min; detector, UV 254 nm. This resulted in 5-[(2-amino-3-fluoropyridin-4-yl)methyl]-4-chloro-N-(4-chloro-2-fluorophenyl)pyridin-3-amine (133 mg). LCMS: (ESI, m/z): [M+1]⁺=381.0. ¹H NMR (400 MHz, Chloroform-d) δ 8.37 (s, 1H), 8.06 (s, 1H), 7.74 (d, J=5.3 Hz, 1H), 7.28 (d, J=8.7 Hz, 1H), 7.20 (m, 1H), 7.11 (m, 1H), 6.41 (m, 1H), 5.94 (s, 1H), 4.82 (s, 2H), 4.09 (s, 2H). ¹⁹F NMR (377 MHz, Chloroform-d) δ −125.08, −144.49.

Step 5: To a stirred solution of 5-[(2-amino-3-fluoropyridin-4-yl)methyl]-4-chloro-N-(4-chloro-2-fluorophenyl)pyridin-3-amine (50 mg, 0.131 mmol) in DMA (2 mL) was added pyridine (103.75 mg, 1.310 mmol, 10 equiv) and N-methylsulfamoyl chloride (20.39 mg, 0.157 mmol, 1.2 equiv) in DMA (0.5 mL) dropwise at 0° C. under nitrogen atmosphere. The resulting mixture was stirred for 2 h at room temperature under nitrogen atmosphere. The reaction mixture was purified by reversed-phase flash chromatography with the following conditions: column, C18 silica gel; mobile phase, ACN in water (10 mmol/L NH₄HCO₃), 5% to 60% gradient in 25 min; detector, UV 254 nm. This resulted in 4-chloro-N-(4-chloro-2-fluorophenyl)-5-({3-fluoro-2-[(methylsulfamoyl)amino]pyridin-4-yl}methyl)pyridin-3-amine (51.5 mg). LCMS: (ESI, m/z): [M+1]⁺=474.0. ¹H NMR (300 MHz, Methanol-d₄) δ 8.02 (s, 1H), 8.00-7.94 (m, 2H), 7.34-7.26 (m, 1H), 7.19 (m, 2H), 6.78 (m, 1H), 4.22 (s, 2H), 2.63 (s, 3H). ¹⁹F NMR (282 MHz, Methanol-d₄) δ −122.52, −142.24.

### Example 68: 4-[[5-(allylamino)-4-methyl-3-pyridyl]methyl]-3-fluoro-N-(methylsulfamoyl)pyridin-2-amine

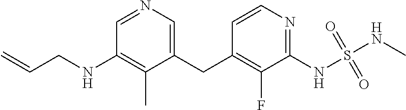 [SMILES: C=CCNc1cncc(Cc2ccnc(NS(=O)(=O)NC)c2F)c1C]

### Route

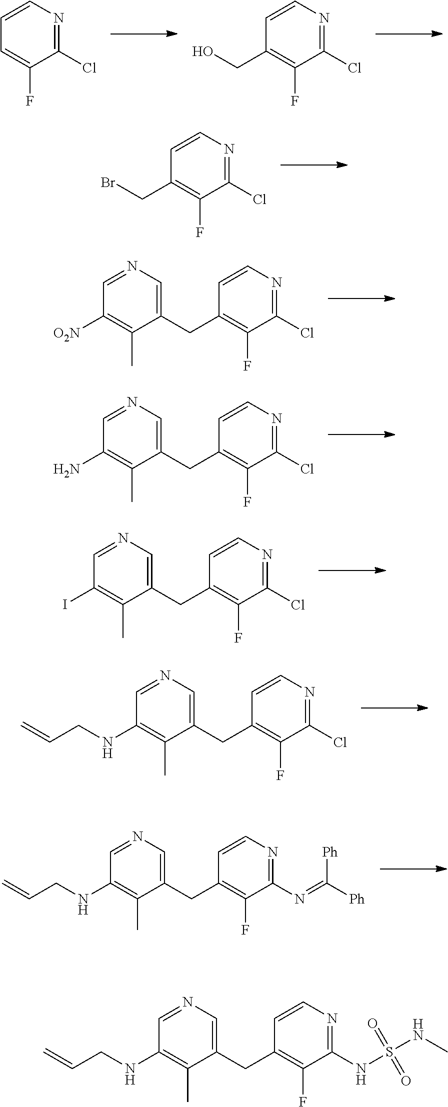

Step 1: To a solution of 2-chloro-3-fluoro-pyridine (15 g, 114.04 mmol) in THF (150 mL) was slowly dropped LDA (2 M, 68.42 mL) at −78° C. for 1 h. N,N-dimethylformamide (83.35 g, 1.14 mol, 87.74 mL) was added dropwise at −78° C. under N₂. The mixture was warm to 0° C. and stirred for 2 h. Then NaBH₄ (5.61 g, 148.21 mmol) in batches were added at 0° C. The mixture was stirred at 0° C. for 2 h. The mixture was poured into water (100 mL), and extracted with EtOAc (100 mL×2). The extract was washed with 1.0 M aqueous HCl (10 mL×3), saturated aqueous NaHCO₃ (10 mL×4), and brine (20 mL). The organic layer was dried over Na₂SO₄ and concentrated. The residue was purified by flash chromatography on silica gel (ethyl acetate in petroleum ether=0-25%) to give (2-chloro-3-fluoro-4-pyridyl) methanol (8.2 g, 50.75 mmol). ¹H NMR (400 MHz, CDCl₃) δ=8.17 (d, J=4.8 Hz, 1H), 7.46 (t, J=4.8 Hz, 1H), 4.85 (s, 2H), 2.80-2.70 (m, 1H). ¹⁹F NMR (376.5 MHz, CDCl₃) δ=−125.606.

Step 2: To a solution of (2-chloro-3-fluoro-4-pyridyl) methanol (4 g, 24.76 mmol) in DCM (45 mL) was added PBr₃ (10.05 g, 37.14 mmol, 873 mL) dropwise at 0° C. under N₂. The mixture was stirred at 25° C. for 12 h. The mixture was added dropwise into H₂O (20 mL) at 0° C. The mixture was adjusted to pH=7 with saturated NaHCO₃ solution slowly at 0° C. and the aqueous layer was extracted with DCM (50 mL×3). The combined organic layer was washed with brine (50 mL), dried over anhydrous Na₂SO₄, filtered and concentrated to give 4-(bromomethyl)-2-chloro-3-fluoro-pyridine (4.6 g, 20.49 mmol). ¹H NMR (400 MHz, CDCl₃) δ=8.23-8.15 (m, 1H), 7.31 (t, J=4.8 Hz, 1H), 4.44 (s, 2H). ¹⁹F NMR (376.5 MHz, CDCl₃) δ=−123.512.

Step 3: To a solution of 4-(bromomethyl)-2-chloro-3-fluoro-pyridine (2 g, 8.91 mmol) in toluene (20 mL), EtOH (5 mL) and H₂O (2 mL) were added 4-methyl-3-nitro-5-(4,4,5,5-tetramethyl-1,3,2-dioxaborolan-2-yl)pyridine (1.57 g, 5.94 mmol), Pd(PPh₃)₄ (686.43 mg, 594.02 mmol) and Na₂CO₃ (1.26 g, 11.88 mmol). The mixture was stirred at 80° C. for 1 h. The mixture was combined with another batch of 2.6 g (3). Water (40 mL) was added. The mixture were extracted with EtOAc (100 mL×3). The combined organic layers were washed with brine (30 mL), dried over anhydrous Na₂SO₄, filtered and concentrated. The crude product was purified by flash chromatography on silica gel (EtOAc in Petroleum ether=0-30%) to give 2-chloro-3-fluoro-4-[(4-methyl-5-nitro-3-pyridyl)methyl]pyridine (3.4 g, 12.07 mmol). ¹H NMR (400 MHz, CDCl₃) δ=8.99 (s, 1H), 8.59 (s, 1H), 8.14 (d, J=4.8 Hz, 1H), 6.84 (t, J=4.8 Hz, 1H), 4.17 (s, 2H), 2.43 (s, 3H). ¹⁹F NMR (376.5 MHz, CDCl₃) δ=−122.944.

Step 4: To a solution of 2-chloro-3-fluoro-4-[(4-methyl-5-nitro-3-pyridyl)methyl]pyridine (180 mg, 639.05 mmol) in EtOH (2 mL) and EtOAc (2 mL) was added SnCl₂·2H₂O (1.01 g, 4.47 mmol). The mixture was stirred at 90° C. for 6 h. After cooling to room temperature, the mixture was concentrated, sat. NaHCO₃ solution was added to the reaction mixture until pH=10. Then the reaction mixture was poured into water (20 mL) and extracted with EtOAc (20 mL×3). The combined organic layers were washed with brine (20 mL), dried over anhydrous Na₂SO₄, filtered and concentrated. The crude product was purified by flash chromatography on silica gel (EtOAc in Petroleum ether=0-100%) to give 5-[(2-chloro-3-fluoro-4-pyridyl)methyl]-4-methyl-pyridin-3-amine (80 mg, 317.86 mmol). ¹H NMR (400 MHz, CDCl₃) δ=8.07-8.00 (m, 2H), 7.88 (s, 1H), 6.78 (t, J=4.8 Hz, 1H), 4.02 (s, 2H), 3.70-3.62 (m, 2H), 1.99 (s, 3H). ¹⁹F NMR (376.5 MHz, CDCl₃) δ=−123.829.

Step 5: To a solution of TsOH (1.42 g, 8.26 mmol) in MeCN (5 mL) was added 5-[(2-chloro-3-fluoro-4-pyridyl)methyl]-4-methyl-pyridin-3-amine (520 mg, 2.07 mmol) at 0° C. After stirred for 10 mins. A solution of NaNO₂ (427.65 mg, 6.20 mmol) and KI (1.20 g, 7.23 mmol) in H₂O (1.2 mL) was added at 0° C. The mixture was stirred at 25° C. for 6 h. Water (30 mL) was added. The mixture were extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-20%) to give 2-chloro-3-fluoro-4-[(5-iodo-4-methyl-3-pyridyl)methyl]pyridine (530 mg, 1.46 mmol). ¹H NMR (400 MHz, CDCl₃) δ=8.85 (s, 1H), 8.27 (s, 1H), 8.09 (d, J=5.2 Hz, 1H), 6.79 (t, J=5.2 Hz, 1H), 4.11 (s, 2H), 2.34 (s, 3H). ¹⁹F NMR (376.5 MHz, CDCl₃) δ=−123.374.

Step 6: To a solution of 2-chloro-3-fluoro-4-[(5-iodo-4-methyl-3-pyridyl)methyl]pyridine (530 mg, 1.46 mmol) in DMF (10 mL) were added prop-2-en-1-amine (232.49 mg, 2.49 mmol, 305.50 μL, HCl), CuI (250.56 mg, 1.32 mmol), 2-(2-methylpropanoyl)cyclohexanone (442.66 mg, 2.63 mmol) and Cs₂CO₃ (2.38 g, 7.31 mmol). The mixture was stirred at 85° C. for 12 h. Water (50 mL) was added. The mixture were extracted with EtOAc (30 mL×3). The aqueous layer were washed with H₂O (30 mL×5). The combined organic layers were washed with brine (30 mL), dried over anhydrous Na₂SO₄, filtered and concentrated. The crude product was purified by flash chromatography on silica gel (EtOAc in Petroleum ether=0-98%) to give N-allyl-5-[(2-chloro-3-fluoro-4-pyridyl)methyl]-4-methyl-pyridin-3-amine (150 mg, 514.14 μmol). ¹H NMR (400 MHz, CDCl₃) δ=8.12-8.06 (m, 1H), 8.05-7.92 (m, 1H), 6.86-6.63 (m, 2H), 6.07-5.82 (m, 1H), 5.37-5.17 (m, 2H), 4.60-4.31 (m, 2H), 4.19-3.91 (m, 2H), 1.95-1.70 (m, 3H). ¹⁹F NMR (376.5 MHz, CDCl₃) δ=−123.540.

Step 7: To a solution of N-allyl-5-[(2-chloro-3-fluoro-4-pyridyl)methyl]-4-methyl-pyridin-3-amine (150 mg, 514.14 μmol) and diphenylmethanimine (102.50 mg, 565.55 μmol, 94.90 μL) in toluene (2 mL) were added tBuONa (98.82 mg, 1.03 mmol), Pd₂(dba)₃ (94.16 mg, 102.83 μmol) and BINAP (128.06 mg, 205.66 μmol). The mixture was stirred at 80° C. for 2 h. The mixture was concentrated to give N-allyl-5-[[2-(benzhydrylideneamino)-3-fluoro-4-pyridyl]methyl]-4-methyl-pyridin-3-amine (224.43 mg, 514.13 μmol). LCMS Rₜ=0.819 min 1.5 min chromatography, 5-95AB, ESI calcd. for C₂₈H₂₆FN₄ [M+H]⁺437.2, found 437.2.

Step 8: To a solution of N-allyl-5-[[2-(benzhydrylideneamino)-3-fluoro-4-pyridyl]methyl]-4-methyl-pyridin-3-amine (224.43 mg, 514.13 μmol) in MeOH (1 mL) was added HCl/MeOH (4 M, 3 mL, 12.00 mmol). The mixture was stirred at 25° C. for 2 h. NH₃/MeOH (20 mL) was added. The mixture was concentrated. The crude product was purified by flash chromatography on silica gel (EtOAc in Petroleum ether=0-100%) and Prep-TLC (DCM:MeOH=10:1) to give 4-[[5-(allylamino)-4-methyl-3-pyridyl]methyl]-3-fluoro-pyridin-2-amine (30 mg, 110.16 μmol). ¹H NMR (400 MHz, CDCl₃) δ=7.58 (d, J=8.0 Hz, 1H), 7.17-7.12 (m, 1H), 6.93-6.81 (m, 2H), 6.05-5.90 (m, 1H), 5.35-5.18 (m, 2H), 4.78-4.62 (m, 2H), 3.98-3.80 (m, 4H), 1.99 (s, 3H). ¹⁹F NMR (376.5 MHz, CDCl₃) δ=−145.481.

Step 9: To a solution of 4-[[5-(allylamino)-4-methyl-3-pyridyl]methyl]-3-fluoro-pyridin-2-amine (30 mg, 110.16 μmol) in MeCN (1 mL) and DMA (0.3 mL) were added Py (87.14 mg, 1.10 mmol, 88.92 μL) and (sulfamoylamino) methane (72.80 mg, 660.99 μmol). The mixture was stirred at 25° C. for 2 h. The mixture was concentrated. The crude product was purified by Prep-HPLC (column: Welch Xtimate C18 150×30 mm×5 μm; mobile phase: [water(NH₃H₂O+NH₄HCO₃)−ACN]; gradient: 25%-55% B over 7 min) to give 4-[[5-(allylamino)-4-methyl-3-pyridyl]methyl]-3-fluoro-N-(methylsulfamoyl)pyridin-2-amine (2.6 mg, 7.12 μmol). ¹H NMR (400 MHz, CD₃OD) δ=7.92 (d, J=4.8 Hz, 1H), 7.71 (d, J=18.4 Hz, 2H), 6.64-6.55 (m, 1H), 6.02-5.84 (m, 1H), 5.29-5.10 (m, 2H), 4.06 (s, 2H), 3.90-3.85 (m, 2H), 2.62 (s, 3H), 2.05 (s, 3H). ¹⁹F NMR (376.5 MHz, CD₃OD) δ=−142.530. LCMS Rₜ=0.576 min 1.5 min chromatography, 5-95AB, ESI calcd. for C₁₆H₂₁FN₅O₂S [M+H]⁺366.1, found 366.0.

### Example 69: 3-fluoro-N-(methylsulfamoyl)-4-[[4-methyl-5-[[5-(trifluoromethoxy)-2-pyridyl]amino]-3-pyridyl]methyl]pyridin-2-amine

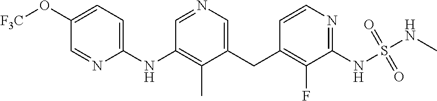 [SMILES: CNS(=O)(=O)Nc1nccc(Cc2cncc(Nc3ccc(OC(F)(F)F)cn3)c2C)c1F]

### Route

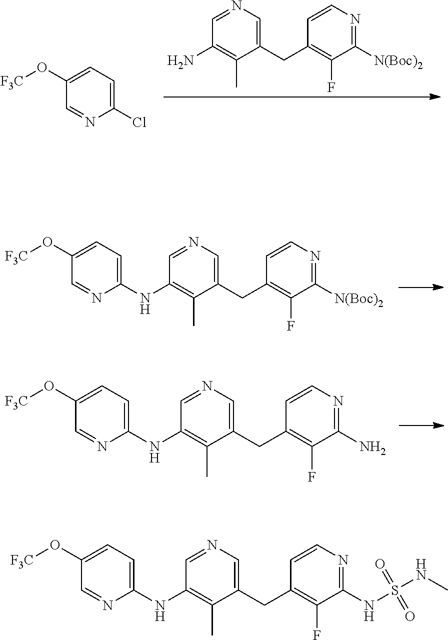

Step 1:2-chloro-5-(trifluoromethoxy), pyridine (342.57 mg, 1.73 mmol) and Pd₂(dba)₃ (52.93 mg, 57.81 μmol) were added to a solution of tert-butyl N-[4-[(5-amino-4-methyl-3-pyridyl)methyl]-3-fluoro-2-pyridyl]-N-tert-butoxycarbonyl-carbamate (500 mg, 1.16 mmol) in dioxane (5 mL) under nitrogen. The mixture was degassed for 5 minutes under N₂ before adding Xantphos (66.89 mg, 115.61 μmol) and Cs₂CO₃ (527.35 mg, 1.62 mmol). The mixture was stirred at 100° C. for 24 hours. The mixture was concentrated. The mixture was purified by flash chromatography on silica gel (EtOAc in petroleum ether=0-50%) to afford tert-butyl N-tert-butoxycarbonyl-N-[3-fluoro-4-[[4-methyl-5-[[5-(trifluoromethoxy)-2-pyridyl]amino]-3-pyridyl]methyl]-2-pyridyl]carbamate (200 mg, 336.95 μmol). LCMS Rt=1.77 min in 3 min chromatography, 5-95 CD, ESI calcd. for C₂₈H₃₂F₄N₅O₅ [M+H]⁺594.2, found 594.2.

Step 2: A solution of tert-butyl N-tert-butoxycarbonyl-N-[3-fluoro-4-[[4-methyl-5-[[5-(trifluoromethoxy)-2-pyridyl]amino]-3-pyridyl]methyl]-2-pyridyl]carbamate (200 mg, 336.95 umol) in HCl/MeOH (5 mL, 4 M) was stirred at 20° C. for 3 h. The mixture was concentrated to afford 3-fluoro-4-[[4-methyl-5-[[5-(trifluoromethoxy)-2-pyridyl]amino]-3-pyridyl]methyl]pyridin-2-amine (132.5 mg, 336.86 μmol). LCMS Rt=1.32 min in 3 min chromatography, 5-95 CD, ESI calcd. for C₁₈H₁₆F₄N₅O [M+H]⁺394.3, found 394.1.

Step 3: To a solution of 3-fluoro-4-[[4-methyl-5-[[5-(trifluoromethoxy)-2-pyridyl]amino]-3-pyridyl]methyl]pyridin-2-amine (100 mg, 254.23 μmol) in MeCN (3 mL) was added Py (201.10 mg, 2.54 mmol, 205.20 μL) and methylsulfamoyl chloride (164.70 mg, 1.27 mmol) at 0° C. The mixture was stirred at 25° C. for 2 h. The mixture was concentrated. The mixture was purified by Prep-HPLC (column: Phenomenex C18 80×40 mm×3 um; mobile phase: [water(NH₃H₂O+NH₄HCO₃)−ACN]; gradient: 30%-60% B over 7 min) to give 3-fluoro-N-(methylsulfamoyl)-4-[[4-methyl-5-[[5-(trifluoromethoxy)-2-pyridyl]amino]-3-pyridyl]methyl]pyridin-2-amine (20.5 mg, 42.14 μmol). ¹H NMR (400 MHz, DMSO-d₆) δ=10.37 (s, 1H), 8.81-8.45 (m, 2H), 8.28-7.94 (m, 3H), 7.62 (d, J=2.4, 8.8 Hz, 1H), 7.01 (d, J=4.4 Hz, 1H), 6.87-6.69 (m, 2H), 4.06 (s, 2H), 2.51 (s, 3H), 2.08 (s, 3H). ¹⁹F NMR (376.5 MHz, DMSO-d₆) δ=−57.822, −142.627. LCMS Rt=0.687 min in 1.5 min chromatography, 5-95AB, ESI calcd. for C₁₉H₁₉F₄N₆O₃S [M+H]⁺487.1, found 487.1.

### Example 70: 4-[[3-(4-chloro-2-fluoro-anilino)-2-methyl-phenyl]methyl]-3-fluoro-N-(methylsulfamoyl)pyridin-2-amine

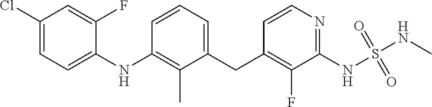 [SMILES: CNS(=O)(=O)Nc1nccc(Cc2cccc(Nc3ccc(Cl)cc3F)c2C)c1F]

### Route

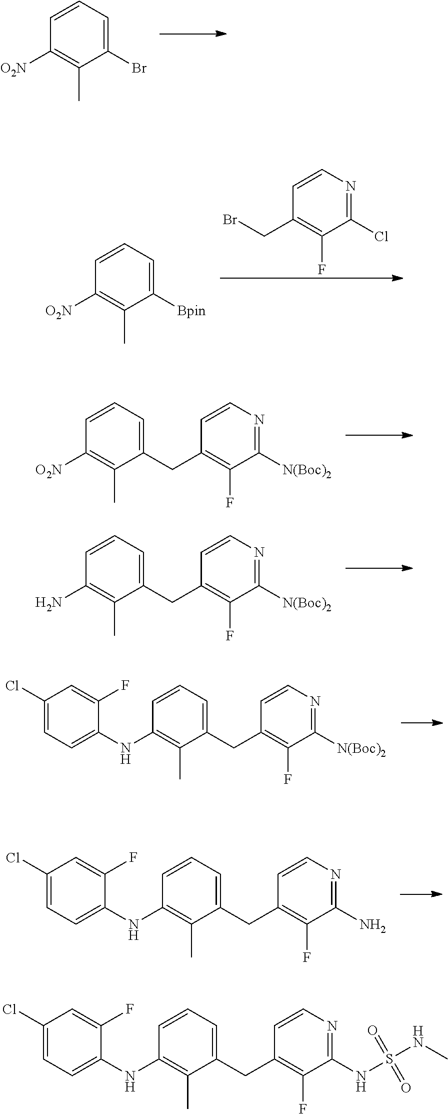

Step 1: To a solution of 1-bromo-2-methyl-3-nitro-benzene (2 g, 9.26 mmol) and 4,4,5,5-tetramethyl-2-(4,4,5,5-tetramethyl-1,3,2-dioxaborolan-2-yl)-1,3,2-dioxaborolane (4.70 g, 18.52 mmol) in dioxane (20 mL) were added KOAc (2.73 g, 27.77 mmol) and Pd(dppf)Cl₂ (338.70 mg, 462.89 μmol). The mixture was stirred at 100° C. for 4 hr. The reaction was concentrated. Water (20 mL) was added and the aqueous was extracted with DCM (2×10 mL). The combined organic layers were dried over anhydrous Na₂SO₄, filtered and concentrated. The residue was purified by flash chromatography on silica gel (Ethyl acetate in Petroleum ether=0-5%) to afford 4,4,5,5-tetramethyl-2-(2-methyl-3-nitro-phenyl)-1,3,2-dioxaborolane (1.4 g, 5.32 mmol). ¹H NMR (400 MHz, CDCl₃) δ −7.94 (d, J=7.6 Hz, 1H), 7.80 (d, J=8.0 Hz, 1H), 7.29 (t, J=7.6 Hz, 1H), 2.67 (s, 3H), 1.36 (s, 12H)

Step 2: To a solution of tert-butyl N-[4-(bromomethyl)-3-fluoro-2-pyridyl]-N-tert-butoxycarbonyl-carbamate (500 mg, 1.23 mmol) and 4,4,5,5-tetramethyl-2-(2-methyl-3-nitro-phenyl)-1,3,2-dioxaborolane (649.21 mg, 2.47 mmol) in Tol. (8 mL), EtOH (2 mL) and H₂O (1 mL) were added Pd(PPh₃)₄ (71.29 mg, 61.69 μmol) and K₂CO₃ (511.55 mg, 3.70 mmol). The mixture was stirred at 85° C. for 12 hr. The reaction was concentrated. Water (5 mL) was added and the aqueous was extracted with DCM (2×5 mL). The combined organic layers were dried over anhydrous Na₂SO₄, filtered and concentrated. The residue was purified by flash chromatography on silica gel (Ethyl acetate in Petroleum ether=0-30%) to afford tert-butyl N-tert-butoxycarbonyl-N-[3-fluoro-4-[(2-methyl-3-nitro-phenyl)methyl]-2-pyridyl]carbamate (520 mg, 1.13 mmol). LCMS Rt=0.722 min in 1.0 min chromatography, 5-95 CD, ESI calcd. for C₂₃H₂₉FN₃O₆ [M+H]⁺462.2, found 462.2.

Step 3: To a solution of tert-butyl N-tert-butoxycarbonyl-N-[3-fluoro-4-[(2-methyl-3-nitro-phenyl)methyl]-2-pyridyl]carbamate (520 mg, 1.13 mmol) in EtOH (5 mL) and H₂O (1 mL) were added Fe (314.63 mg, 5.63 mmol) and NH₄Cl (602.74 mg, 11.27 mmol). The mixture was stirred at 85° C. for 1 hr. The reaction was concentrated. The residue was purified by flash chromatography on silica gel (Ethyl acetate in Petroleum ether=0-30%) to afford tert-butyl N-[4-[(3-amino-2-methyl-phenyl)methyl]-3-fluoro-2-pyridyl]-N-tert-butoxycarbonyl-carbamate (160 mg, 370.80 μmol). LCMS Rt=0.565 min in 1.0 min chromatography, 5-95AB, ESI calcd. for C₂₃H₃₀FN₃O₄ [M+H]⁺432.2, found 432.2.

Step 4: To a solution of tert-butyl N-[4-[(3-amino-2-methyl-phenyl)methyl]-3-fluoro-2-pyridyl]-N-tert-butoxycarbonyl-carbamate (150 mg, 347.63 μmol) and 4-chloro-2-fluoro-1-iodo-benzene (98.06 mg, 382.39 μmol) in dioxane (1 mL) were added Pd(OAc)₂ (7.80 mg, 34.76 μmol), Cs₂CO₃ (339.79 mg, 1.04 mmol) and Xantphos (20.11 mg, 34.76 μmol). The mixture was stirred at 100° C. for 12 hr. The reaction was concentrated. Water (4 mL) was added and the aqueous was extracted with DCM (2×4 mL). The combined organic layers were dried over anhydrous Na₂SO₄, filtered and concentrated. The residue was purified by flash chromatography on silica gel (Ethyl acetate in Petroleum ether=0-25%) to afford tert-butyl N-tert-butoxycarbonyl-N-[4-[[3-(4-chloro-2-fluoro-anilino)-2-methyl-phenyl]methyl]-3-fluoro-2-pyridyl]carbamate (150 mg, 267.84 μmol). LCMS Rt=0.822 min in 1.0 min chromatography, 5-95AB, ESI calcd. for C₂₉H₃₃ClF₂N₃O₄ [M+H]⁺560.2, found 560.2.

### Example 71: {[3-fluoro-4-({4-methyl-5-[4-(methylsulfanyl)phenoxy]pyridin-3-yl}methyl)pyridin-2-yl]sulfamoyl}(methyl)amine

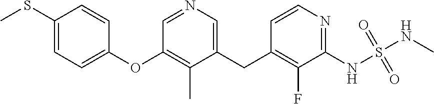

### Route

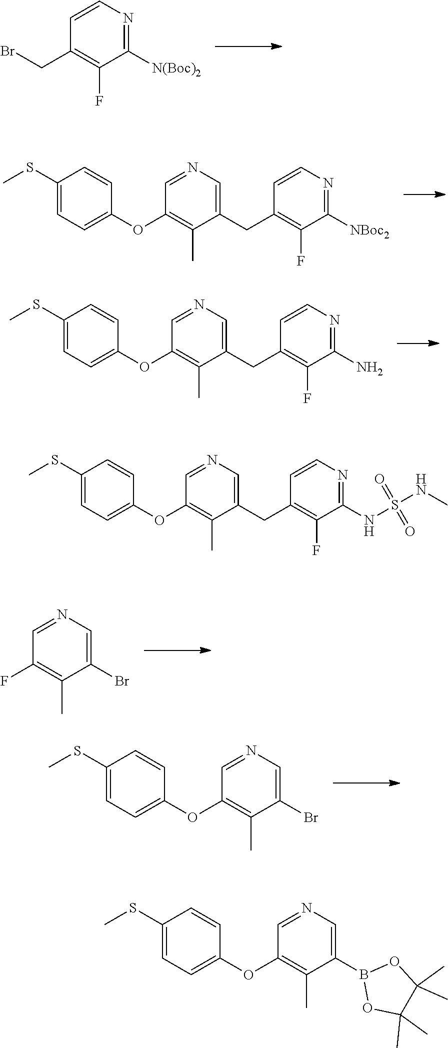

Step 1: To a stirred solution of 4-(methylthio)-phenol (368.92 mg, 2.631 mmol) and 3-bromo-5-fluoro-4-methylpyridine (500 mg, 2.631 mmol) in DMF (5 mL) was added K₂CO₃ (727.34 mg, 5.262 mmol) in portions at room temperature under air atmosphere. And then keep stirring for 48 h at 110° C. The reaction mixture was diluted with water (20 mL). The resulting mixture was extracted with EA (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (10:1) to afford 3-bromo-4-methyl-5-[4-(methylsulfanyl)phenoxy]pyridine (231 mg). LCMS: (ESI, m/z): [M+1]⁺=309.90. ¹H NMR (400 MHz, Chloroform-d) δ 8.48 (s, 1H), 8.09 (s, 1H), 7.29-7.26 (m, 2H), 6.90-6.85 (m, 2H), 2.48 (s, 3H), 2.36 (s, 3H

Step 2: To a stirred mixture of 3-bromo-4-methyl-5-[4-(methylsulfanyl)phenoxy]pyridine (400 mg, 1.289 mmol) and bis(pinacolato)diboron (491.16 mg, 1.933 mmol, 1.5 equiv) in dioxane (8 mL) were added AcOK (379.65 mg, 3.867 mmol, 3 equiv) and Pd(dppf)Cl₂ (94.35 mg, 0.129 mmol, 0.1 equiv). After stirring for 16 h at 100° C. under a nitrogen atmosphere, desired product could be detected by LCMS. The resulting mixture was filtered, the filter cake was washed with EA. The filtrate was concentrated under reduced pressure. This resulted in 4-methyl-3-[4-(methylsulfanyl) phenoxy]-5-(4,4,5-trimethyl-1,3,2-dioxaborolan-2-yl)pyridine (770 mg). LCMS: (ESI, m/z): [M+1]⁺=358.10

Step 3: To a stirred mixture of tert-butyl N-[4-(bromomethyl)-3-fluoropyridin-2-yl]-N-(tert-butoxycarbonyl) carbamate (250 mg, 0.617 mmol) and 4-methyl-3-[4-(methylsulfanyl) phenoxy]-5-(4,4,5,5-tetramethyl-1,3,2-dioxaborolan-2-yl)pyridine (330.60 mg, 0.925 mmol, 1.5 equiv) in dioxane (5 mL) and H₂O (0.5 mL) were added K₂CO₃ (255.77 mg, 1.851 mmol, 3 equiv) and Pd(dppf)Cl₂ (45.14 mg, 0.062 mmol, 0.1 equiv). The resulting mixture was stirred for 2 h at 80° C. under nitrogen atmosphere. Desired product could be detected by LCMS. The resulting mixture was diluted with water (20 mL). The resulting mixture was extracted with EA (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (1:1) to afford tert-butyl N-(tert-butoxycarbonyl)-N-[3-fluoro-4-({4-methyl-5-[4-(methylsulfanyl)phenoxy]pyridin-3-yl}methyl)pyridin-2-yl]carbamate (180 mg). LCMS: (ESI, m/z): [M+1]⁺=556.20. ¹H NMR (400 MHz, Chloroform-d) δ 8.22-8.18 (m, 2H), 8.14 (s, 1H), 7.28 (d, J=2.1 Hz, 1H), 6.93 (m, 1H), 6.89-6.84 (m, 2H), 5.30 (s, 1H), 4.11 (s, 2H), 2.48 (s, 3H), 2.16 (s, 3H), 1.42 (s, 18H). ¹⁹F NMR (376 MHz, Chloroform-d) δ −131.01.

Step 4: To a stirred solution of tert-butyl N-(tert-butoxycarbonyl)-N-[3-fluoro-4-({4-methyl-5-[4-(methylsulfanyl)phenoxy]pyridin-3-yl}methyl)pyridin-2-yl]carbamate (160 mg, 0.288 mmol) in DCM (2 mL) was added TFA (0.5 mL) dropwise at 0° C. under nitrogen atmosphere. The resulting mixture was stirred for additional 1 h at room temperature. Desired product could be detected by LCMS. The reaction mixture was concentrated under reduced pressure. The mixture was basified to pH 10 with sat. NaHCO₃ (aq.). The resulting mixture was extracted with EA (3×5 mL). The combined organic layers were washed with brine (5 mL), dried over anhydrous Na₂SO₄. After filtration, the filtrate was concentrated under reduced pressure. The residue was purified by silica gel column chromatography, eluted with CH₂Cl₂/MeOH (20:1) to afford 3-fluoro-4-({4-methyl-5-[4-(methylsulfanyl)phenoxy]pyridin-3-yl}methyl)pyridin-2-amine (76 mg).

LCMS: (ESI, m/z): [M+1]⁺=356.10. ¹H NMR (400 MHz, Chloroform-d) δ 8.22 (s, 1H), 8.13 (s, 1H), 7.72 (d, J=5.3 Hz, 1H), 7.27 (d, J=2.2 Hz, 1H), 7.25 (s, 1H), 6.89-6.84 (m, 2H), 6.31 (t, J=5.2 Hz, 1H), 4.97 (s, 2H), 4.02 (s, 2H), 2.47 (s, 3H), 2.17 (s, 3H). ¹⁹F NMR (377 MHz, Chloroform-d) δ −144.50.

Step 5: To a stirred solution of 3-fluoro-4-({4-methyl-5-[4-(methylsulfanyl)phenoxy]pyridin-3-yl}methyl)pyridin-2-amine (30 mg, 0.084 mmol) and pyridine (33.38 mg, 0.420 mmol, 5 equiv) in DMA (0.4 mL) was added and N-methylsulfamoyl chloride (13.12 mg, 0.101 mmol, 1.2 equiv) in DMA (0.1 mL) dropwise at 0° C. under nitrogen atmosphere. The resulting mixture was stirred for 2 h at room temperature under nitrogen atmosphere. Desired product could be detected by LCMS. The residue was purified by reversed-phase flash chromatography with the following conditions: column, C18 silica gel; mobile phase, ACN in Water (10 mmol/L NH₄HCO₃), 5% to 60% gradient in 25 min; detector, UV 254 nm. This resulted in {[3-fluoro-4-({4-methyl-5-[4-(methylsulfanyl)phenoxy]pyridin-3-yl}methyl)pyridin-2-yl]sulfamoyl}(methyl)amine (30.7 mg). LCMS: (ESI, m/z): [M+1]⁺=449.20. ¹H NMR (300 MHz, Chloroform-d) δ 8.21 (s, 1H), 8.15 (s, 1H), 7.97 (d, J=5.2 Hz, 1H), 7.28 (d, J=2.1 Hz, 1H), 7.25 (s, 1H), 6.90-6.83 (m, 2H), 6.61 (t, J=5.1 Hz, 1H), 5.47 (d, J=5.9 Hz, 1H), 4.05 (s, 2H), 2.77 (d, J=5.0 Hz, 3H), 2.47 (s, 3H), 2.16 (s, 3H). ¹⁹F NMR (282 MHz, Chloroform-d) δ −142.82.

### Example 72: [(3-fluoro-4-{[5-(4-methanesulfonylphenoxy)-4-methylpyridin-3-yl]methyl}pyridin-2-yl)sulfamoyl](methyl)amine

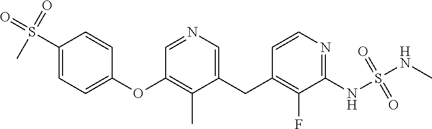 [SMILES: CNS(=O)(=O)Nc1nccc(Cc2cncc(Oc3ccc(S(C)(=O)=O)cc3)c2C)c1F]

### Route

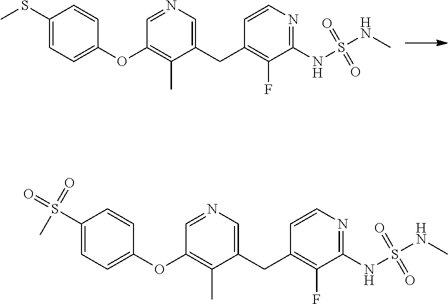

Step 1: To a stirred solution of {[3-fluoro-4-({4-methyl-5-[4-(methylsulfanyl)phenoxy]pyridin-3-yl}methyl)pyridin-2-yl]sulfamoyl}(methyl)amine (15 mg, 0.033 mmol, example 71) in acetone (1 mL), H₂O (1 mL) and MeOH (0.1 mL) was added oxone (22.49 mg, 0.132 mmol, 4 equiv) in portions at 0° C. The resulting mixture was stirred for 4 h at room temperature. The resulting mixture was concentrated under vacuum. The residue was purified by reversed-phase flash chromatography with the following conditions: column, C18 silica gel; mobile phase, ACN in water (10 mmol/L NH₄HCO₃), 5% to 50% gradient in 30 min; detector, UV 254 nm. This resulted in [(3-fluoro-4-{[5-(4-methanesulfonylphenoxy)-4-methylpyridin-3-yl]methyl}pyridin-2-yl)sulfamoyl](methyl)amine (6.6 mg). LCMS: (ESI, m/z): [M+1]⁺=481.20. ¹H NMR (400 MHz, Chloroform-d) δ 8.30 (d, J=19.1 Hz, 2H), 7.99 (d, J=5.1 Hz, 1H), 7.97-7.88 (m, 2H), 7.37 (s, 1H), 7.06-6.98 (m, 2H), 6.64 (t, J=5.1 Hz, 1H), 5.46 (d, J=5.7 Hz, 1H), 4.09 (s, 2H), 3.06 (s, 3H), 2.78 (d, J=5.0 Hz, 3H), 2.15 (s, 3H). ¹⁹F NMR (377 MHz, Chloroform-d) δ −142.558.

### Example 73: 4-[[5-(4,4-dimethylcyclohexoxy)-4-methyl-3-pyridyl]methyl]-3-fluoro-N-(methylsulfamoyl)pyridin-2-amine

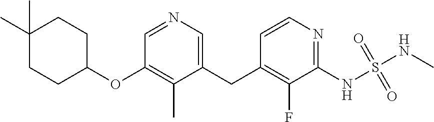

### Route

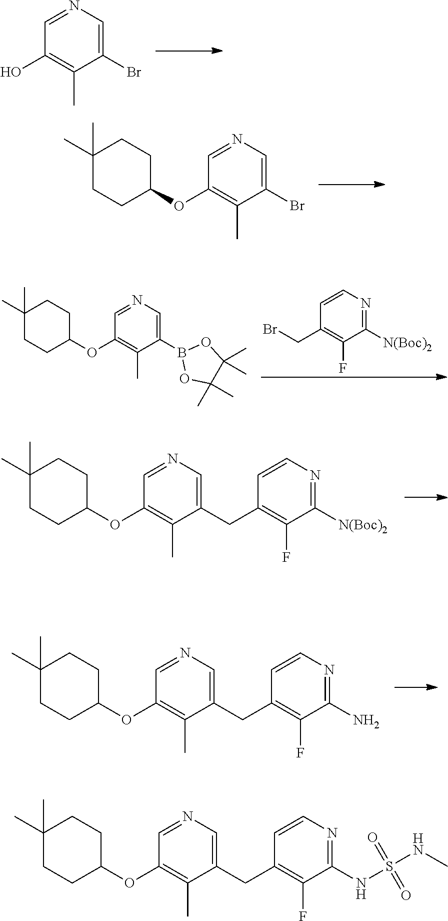

Step 1: To a solution of 5-bromo-4-methyl-pyridin-3-ol (2 g, 10.64 mmol) and 4,4-dimethylcyclohexanol (3.41 g, 26.59 mmol), PPh₃ (6.97 g, 26.59 mmol) in toluene (30 mL) was added DIAD (5.38 g, 26.59 mmol, 5.16 mL) dropwise under N₂ at 0° C. The mixture was stirred at 80° C. for 1 hr. The mixture was added dropwise into sat. NaHCO₃ (100 mL) at 0° C. and the aqueous layer was extracted with EtOAc (50 mL×3). The combined organic layer was washed with brine (100 mL), dried over anhydrous Na₂SO₄, filtered and concentrated. The residue was purified by flash chromatography on silica gel (Ethyl acetate in Petroleum ether=0-4%) to give 3-bromo-5-(4,4-dimethylcyclohexoxy)-4-methyl-pyridine (2.7 g, 9.05 mmol). ¹H NMR (400 MHz, CDCl₃) δ=8.27 (s, 1H), 8.08 (s, 1H), 4.41-4.25 (m, 1H), 2.32 (s, 3H), 1.92-1.82 (m, 2H), 1.80-1.66 (m, 2H), 1.57-1.44 (m, 2H), 1.31-1.20 (m, 2H), 0.97 (s, 3H), 0.95 (s, 3H).

Step 2: A mixture of 3-bromo-5-(4,4-dimethylcyclohexoxy)-4-methyl-pyridine (2.7 g, 9.05 mmol), cyclopentyl(diphenyl) phosphane; dichloropalladium; iron (662.47 mg, 905.38 μmol), 4,4,5,5-tetramethyl-2-(4,4,5,5-tetramethyl-1,3,2-dioxaborolan-2-yl)-1,3,2-dioxaborolane (3.45 g, 13.58 mmol) and KOAc (2.67 g, 27.16 mmol) in dioxane (27 mL) was stirred at 110° C. for 4 h. The mixture was filtered, and the filtrate was concentrated. The residue was purified by flash chromatography on silica gel (Ethyl acetate in Petroleum ether=0-30%) to give 3-(4,4-dimethylcyclohexoxy)-4-methyl-5-(4,4,5,5-tetramethyl-1,3,2-dioxaborolan-2-yl)pyridine (2 g, 5.79 mmol). ¹H NMR (400 MHz, CDCl₃) δ=8.44 (s, 1H), 8.19 (s, 1H), 4.34-4.23 (m, 1H), 2.42 (s, 3H), 1.88-1.82 (m, 2H), 1.76-1.69 (m, 2H), 1.54-1.47 (m, 2H), 1.35 (s, 12H), 1.23-1.18 (m, 2H), 0.97 (s, 3H), 0.94 (s, 3H).

Step 3: A mixture of 3-(4,4-dimethylcyclohexoxy)-4-methyl-5-(4,4,5,5-tetramethyl-1,3,2-dioxaborolan-2-yl)pyridine (800 mg, 2.32 mmol), tert-butyl N-[4-(bromomethyl)-3-fluoro-2-pyridyl]-N-tert-butoxycarbonyl-carbamate (845.06 mg, 2.09 mmol), Na₂CO₃ (736.72 mg, 6.95 mmol) and Pd(PPh₃)₄ (267.74 mg, 231.69 μmol) in toluene (8 mL), EtOH (2 mL) and H₂O (0.5 mL) was stirred at 80° C. for 1 h under N₂. The mixture was filtered, and the filter cake was washed with EtOAc (10 mL×3), the filtrate was concentrated. The residue was purified by flash chromatography on silica gel (Ethyl acetate in Petroleum ether=0-15%) and then purified by flash chromatography on silica gel (MeOH in DCM=0-1%) to give tert-butyl N-tert-butoxycarbonyl-N-[4-[[5-(4,4-dimethylcyclohexoxy)-4-methyl-3-pyridyl]methyl]-3-fluoro-2-pyridyl]carbamate (700 mg, 1.29 mmol). ¹H NMR (400 MHz, CDCl₃) δ=8.19-8.11 (m, 2H), 8.00 (s, 1H), 6.86 (t, J=5.2 Hz, 1H), 4.37-4.26 (m, 1H), 4.03 (s, 2H), 2.08 (s, 3H), 1.92-1.82 (m, 2H), 1.78-1.67 (m, 2H), 1.54-1.46 (m, 2H), 1.41 (s, 18H), 1.29-1.26 (m, 2H), 0.96-0.85 (m, 6H). ¹⁹F NMR (376.5 MHz, CDCl₃) δ=−131.275.

Step 4: A solution of tert-butyl N-tert-butoxycarbonyl-N-[4-[[5-(4,4-dimethylcyclohexoxy)-4-methyl-3-pyridyl]methyl]-3-fluoro-2-pyridyl]carbamate (650 mg, 1.20 mmol) in HCl/MeOH (4 M, 1.49 mL) was stirred at 25° C. for 2 h. The mixture was diluted with MeOH (15 mL). NH₃/MeOH (7 M, 5 mL) was added dropwise at 0° C. under N₂. The mixture was concentrated. The residue was purified by flash chromatography on silica gel (Methanol in Dichloromethane=0-6%) to give 4-[[5-(4,4-dimethylcyclohexoxy)-4-methyl-3-pyridyl]methyl]-3-fluoro-pyridin-2-amine (200 mg, 582.35 μmol). ¹H NMR (400 MHz, CDCl₃) δ=8.12 (s, 1H), 8.01 (s, 1H), 7.70 (d, J=5.2 Hz, 1H), 6.25 (t, J=5.2 Hz, 1H), 4.64 (s, 2H), 4.42-4.23 (m, 1H), 3.94 (s, 2H), 2.13 (s, 3H), 1.91-1.84 (m, 2H), 1.79-1.69 (m, 2H), 1.54-1.47 (m, 2H), 1.31-1.26 (m, 2H), 0.98-0.95 (m, 6H). ¹⁹F NMR (376.5 MHz, CDCl₃) δ=−145.252.

Step 5: To a solution of 4-[[5-(4,4-dimethylcyclohexoxy)-4-methyl-3-pyridyl]methyl]-3-fluoro-pyridin-2-amine (180 mg, 524.11 μmol) and Py (414.57 mg, 5.24 mmol, 423.03 μL) in MeCN (3 mL) was added N-methylsulfamoyl chloride (135.82 mg, 1.05 mmol) under N₂. The mixture was stirred at 25° C. for 1 h. Then N-methylsulfamoyl chloride (135.81 mg, 1.05 mmol) was added. The mixture was stirred at 25° C. for 1 h. The mixture was concentrated. The residue was purified by prep-HPLC (column: Boston Prime C18 150*30 mm*5 μm; mobile phase: [water(NH₃H₂O+NH₄HCO₃)−ACN]; gradient: 46%-76% B over 7 min) to give 4-[[5-(4,4-dimethylcyclohexoxy)-4-methyl-3-pyridyl]methyl]-3-fluoro-N-(methylsulfamoyl)pyridin-2-amine (40.0 mg, 91.63 μmol). ¹H NMR (400 MHz, CDCl₃) δ=8.15 (s, 1H), 8.09-7.92 (m, 2H), 6.60 (t, J=5.2 Hz, 1H), 5.48 (s, 1H), 4.44-4.30 (m, 1H), 4.04 (s, 2H), 2.78 (d, J=3.2 Hz, 3H), 2.23 (s, 3H), 1.93-1.85 (m, 2H), 1.79-1.71 (m, 2H), 1.55-1.49 (m, 2H), 1.34-1.26 (m, 2H), 0.98-0.91 (m, 6H). ¹⁹F NMR (376.5 MHz, CDCl₃) δ=142.521. LCMS Rₜ=0.543 min in 1 min chromatography, 5-95AB, ESI calcd. for C₂₁H₃₀FN₄O₃S [M+H]⁺437.2, found 437.3.

### Example 74: 3-fluoro-4-[[4-methyl-5-(4-methylcyclohexoxy)-3-pyridyl]methyl]-N-(methylsulfamoyl)pyridin-2-amine

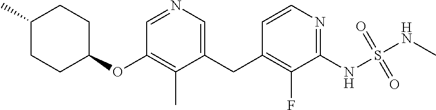

### Route

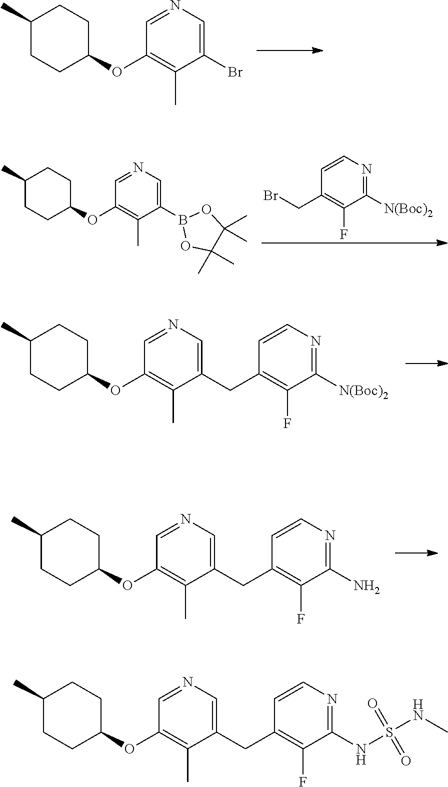

Step 1: A mixture of 3-bromo-4-methyl-5-(4-methylcyclohexoxy)pyridine (1 g, 3.52 mmol, example 63), cyclopentyl(diphenyl) phosphane; dichloropalladium; iron (257.47 mg, 351.88 umol), 4,4,5,5-tetramethyl-2-(4,4,5,5-tetramethyl-1,3,2-dioxaborolan-2-yl)-1,3,2-dioxaborolane (1.34 g, 5.28 mmol) and KOAc (1.04 g, 10.56 mmol) in dioxane (10 mL) was stirred at 110° C. for 4 h. The mixture was filtered and the filter cake was washed with EtOAc (10 mL×3), the filtrate was concentrated. The residue was purified by flash chromatography on silica gel (Ethyl acetate in Petroleum ether=0-40%) to give 4-methyl-3-(4-methylcyclohexoxy)-5-(4,4,5,5-tetramethyl-1,3,2-dioxaborolan-2-yl)pyridine (1 g, 3.02 mmol). ¹H NMR (400 MHz, CDCl₃) δ=8.43 (s, 1H), 8.18 (s, 1H), 4.59 (s, 1H), 2.44 (s, 3H), 2.04-1.96 (m, 2H), 1.61-1.46 (m, 5H), 1.35 (s, 12H), 1.27-1.25 (m, 2H), 0.93 (d, J=5.6 Hz, 3H).

Step 2: A mixture of 4-methyl-3-(4-methylcyclohexoxy)-5-(4,4,5,5-tetramethyl-1,3,2-dioxaborolan-2-yl)pyridine (1 g, 3.02 mmol), tert-butyl N-[4-(bromomethyl)-3-fluoro-2-pyridyl]-N-tert-butoxycarbonyl-carbamate (0.9 g, 2.22 mmol) Na₂CO₃ (959.89 mg, 9.06 mmol), Pd(PPh₃)₄ (348.84 mg, 301.88 μmol) in toluene (12 mL), EtOH (3 mL) and H₂O (0.75 mL) was stirred at 80° C. for 1 h under N₂. The mixture was filtered and the filter cake was washed with EtOAc (10 mL×3), the filtrate was concentrated. The residue was purified by flash chromatography on silica gel (Ethyl acetatein Petroleum ether=0-40%) to give tert-butyl N-tert-butoxycarbonyl-N-[3-fluoro-4-[[4-methyl-5-(4-methylcyclohexoxy)-3-pyridyl]methyl]-2-pyridyl]carbamate (1 g, 1.89 mmol). ¹H NMR (400 MHz, CDCl₃) δ=8.17-8.10 (m, 2H), 7.99 (s, 1H), 6.87 (t, J=4.4 Hz, 1H), 4.62 (s, 1H), 4.03 (s, 2H), 2.10 (s, 3H), 2.02-1.96 (m, 2H), 1.58-1.49 (m, 5H), 1.40 (s, 18H), 1.27-1.23 (m, 2H), 0.92 (d, J=5.6 Hz, 3H) ¹⁹F NMR (376.5 MHz, CDCl₃) δ=−131.279.

Step 3: A mixture of tert-butyl N-tert-butoxycarbonyl-N-[3-fluoro-4-[[4-methyl-5-(4-methylcyclohexoxy)-3-pyridyl]methyl]-2-pyridyl]carbamate (950.00 mg, 1.79 mmol), HCl/MeOH (4 M, 2.24 mL) was stirred at 25° C. for 1 h under N₂. The mixture was diluted with MeOH (15 mL). NH₃/MeOH (7 M, 8 mL) was added dropwise at 0° C. under N₂. The mixture was concentrated. The residue was purified by flash chromatography on silica gel (Methanol in Dichloromethane=0-6%) to give 3-fluoro-4-[[4-methyl-5-(4-methylcyclohexoxy)-3-pyridyl]methyl]pyridin-2-amine (300 mg, 910.72 μmol). ¹H NMR (400 MHz, CDCl₃) δ=8.11 (s, 1H), 8.01 (s, 1H), 7.71 (d, J=5.2 Hz, 1H), 6.26 (t, J=5.2 Hz, 1H), 4.69 (s, 2H), 4.62 (s, 1H), 3.95 (s, 2H), 2.17 (s, 3H), 2.06-1.99 (m, 2H), 1.70-1.44 (m, 5H), 1.41-1.30 (m, 2H), 0.94 (d, J=6.0 Hz, 3H). ¹⁹F NMR (376.5 MHz, CDCl₃) δ=−145.160.

Step 4: To a solution of 3-fluoro-4-[[4-methyl-5-(4-methylcyclohexoxy)-3-pyridyl]methyl]pyridin-2-amine (250 mg, 758.93 μmol) and Py (600.31 mg, 7.59 mmol, 612.57 μL) in MeCN (5 mL) was added N-methylsulfamoyl chloride (196.67 mg, 1.52 mmol) under N₂. The mixture was stirred at 25° C. for 1 h. Then N-methylsulfamoyl chloride (98.33 mg, 758.93 μmol) was added. The mixture was stirred at 25° C. for 1 h. The mixture was concentrated. The residue was purified by prep-HPLC (column: Phenomenex C18 75*30 mm*3 μm; mobile phase: [water(NH₃H₂O+NH₄HCO₃)−ACN]; gradient: 30%-60% B over 8 min) and then purified by prep-HPLC (column: Phenomenex C18 80*40 mm*3 μm; mobile phase: [water(NH₃H₂O+NH₄HCO₃)−ACN]; gradient: 45%-75% B over 7 min) to give 3-fluoro-4-[[4-methyl-5-(4-methylcyclohexoxy)-3-pyridyl]methyl]-N-(methylsulfamoyl)pyridin-2-amine (120 mg, 284.01 μmol). ¹H NMR (400 MHz, CDCl₃) δ=8.14 (s, 1H), 8.01 (s, 1H), 7.96 (d, J=5.2 Hz, 1H), 6.60 (t, J=5.2 Hz, 1H), 5.61-5.30 (m, 1H), 4.64 (s, 1H), 4.03 (s, 2H), 2.78 (s, 3H), 2.21 (s, 3H), 2.08-2.01 (m, 2H), 1.70-1.53 (m, 5H), 1.39-1.30 (m, 2H), 0.94 (d, J=6.0 Hz, 3H). ¹⁹F NMR (376.5 MHz, CDCl₃) δ=142.558. LCMS Rₜ=0.535 min in 1 min chromatography, 5-95AB, ESI calcd. for C₂₀H₂₈FN₄O₃S [M+H]⁺423.1, found 423.2.

### Example: 75: 4-[(5-but-2-ynoxy-4-methyl-3-pyridyl)methyl]-3-fluoro-N-(methylsulfamoyl)pyridin-2-amine

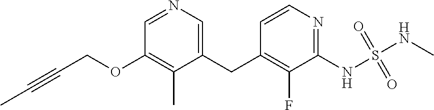 [SMILES: CC#CCOc1cncc(Cc2ccnc(NS(=O)(=O)NC)c2F)c1C]

### Route

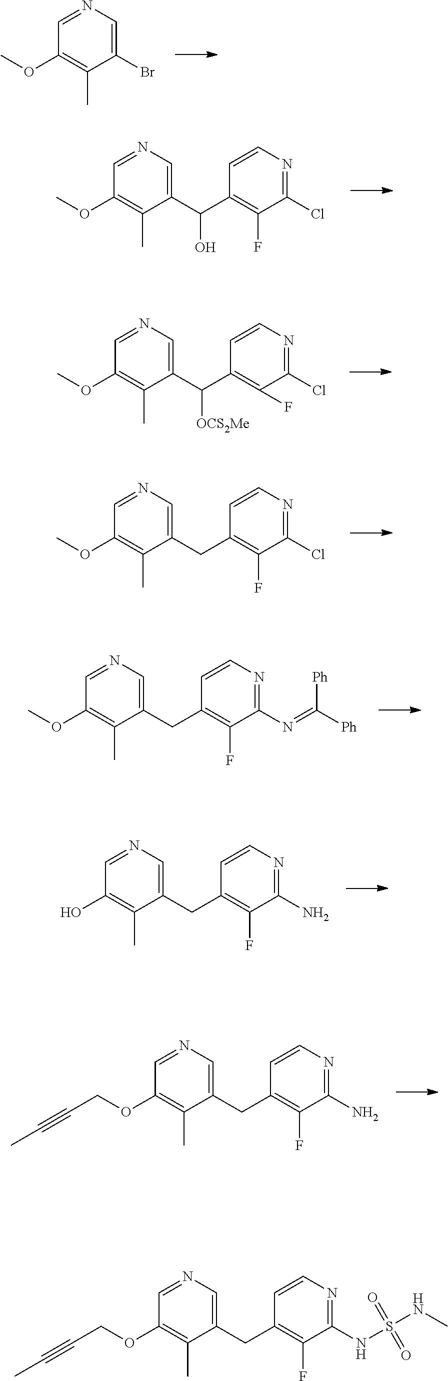

Step 1: To a solution of 3-bromo-5-methoxy-4-methyl-pyridine (5 g, 24.75 mmol) in THF (60 mL) was added n-BuLi (2.5 M, 11.88 mL) at −75° C. The mixture was stirred at −75° C. for 0.5 hr. Then 2-chloro-3-fluoro-pyridine-4-carbaldehyde (4.34 g, 27.22 mmol) was added, the mixture was stirred at 0° C. for 0.5 hr and then the mixture was stirred at 25° C. for 1 hr. Water (50 mL) was added and the mixture were extracted with EtOAc (30 mL×2). The organic layer was dried over anhydrous Na₂SO₄, filtered and concentrated. The crude was purified by flash column chromatography on silica gel (EtOAc in petroleum ether=0-90%) to give (2-chloro-3-fluoro-4-pyridyl)-(5-methoxy-4-methyl-3-pyridyl) methanol (3 g, 10.61 mmol). ¹H NMR (400 MHz, CDCl₃) δ=8.18 (d, J=4.8 Hz, 1H), 7.96 (s, 1H), 7.89 (s, 1H), 7.51 (t, J=4.8, 1H), 6.22 (s, 1H), 3.86 (s, 3H), 2.18 (s, 3H). ¹⁹F NMR (376.5 MHz, CDCl₃) δ=−123.272.

Step 2: To a solution of (2-chloro-3-fluoro-4-pyridyl)-(5-methoxy-4-methyl-3-pyridyl) methanol (6.3 g, 22.29 mmol) in THF (70 mL) were added NaH (2.23 g, 55.71 mmol, 60% purity) and imidazole (22.76 mg, 334.28 mmol) at 0° C. The mixture was stirred at 25° C. for 0.5 hr under N₂. Then CS₂ (21.21 g, 278.57 mmol, 16.79 mL) was added and the mixture was stirred at 25° C. for 1 hr. After CH₃I (38.30 g, 269.86 mmol, 16.80 mL) was added, the mixture was stirred at 25° C. for 0.5 hr. Water (50 mL) was added and the mixture was extracted with DCM (50 mL×2). The organic layer was dried over anhydrous Na₂SO₄, filtered and concentrated. The crude was purified by flash column chromatography on silica gel (EtOAc in petroleum ether=0-25%) to give O-[(2-chloro-3-fluoro-4-pyridyl)-(5-methoxy-4-methyl-3-pyridyl)methyl]methylsulfanylmethanethioate (1.3 g, 3.49 mmol). 1H NMR (400 MHz, CDCl₃) δ=8.17 (d, J=5.2 Hz, 1H), 7.94 (s, 1H), 7.86 (d, J=2.4 Hz, 1H), 7.50 (t, J=4.8 Hz, 1H), 6.20 (s, 1H), 3.84 (s, 3H), 2.17 (s, 3H). ¹⁹F NMR (376.5 MHz, CDCl₃) δ=−123.254.

Step 3: To a solution of O-[(2-chloro-3-fluoro-4-pyridyl)-(5-methoxy-4-methyl-3-pyridyl)methyl]methylsulfanylmethanethioate (1.3 g, 3.49 mmol) in toluene (10 mL) were added AIBN (114.50 mg, 697.30 mmol), tributylstannane (2.57 g, 8.83 mmol, 2.34 mL). The mixture was stirred at 120° C. for 12 hr. Water (20 mL) was added and the mixture was extracted with EtOAc (20 mL×2). The organic layer was dried over anhydrous Na₂SO₄, filtered and concentrated. The crude was purified by flash column chromatography on silica gel (EtOAc in petroleum ether=0-40%) to give 2-chloro-3-fluoro-4-[(5-methoxy-4-methyl-3-pyridyl)methyl]pyridine (520 mg, 1.95 mmol). ¹H NMR (400 MHz, CDCl₃) δ=8.16 (s, 1H), 8.05 (d, J=8.0 Hz, 1H), 8.03 (s, 1H), 6.78 (t, J=5.2 Hz, 1H), 4.03 (s, 2H), 3.92 (s, 3H), 2.07 (s, 3H). ¹⁹F NMR (376.5 MHz, CDCl₃) δ=−123.650.

Step 4: To a solution of 2-chloro-3-fluoro-4-[(5-methoxy-4-methyl-3-pyridyl)methyl]pyridine (420 mg, 1.57 mmol) in toluene (7 mL) were added diphenylmethanimine (342.49 mg, 1.89 mmol, 317.12 mL), NaOBu-t (181.61 mg, 1.89 mmol), BINAP (68.64 mg, 110.24 mmol) and Pd₂(dba)₃ (43.26 mg, 47.24 mmol). The mixture was stirred at 80° C. for 4 hr. Water (20 mL) was added and the mixture were extracted with EtOAc (20 mL×2). The organic layer was dried over anhydrous Na₂SO₄, filtered and concentrated. The residue was purified by flash column chromatography on silica gel (EtOAc in petroleum ether=0-30%) to give N-[3-fluoro-4-[(5-methoxy-4-methyl-3-pyridyl)methyl]-2-pyridyl]-1,1-diphenyl-methanimine (500 mg, 1.22 mmol). LCMS Rt=0.780 min in 1.5 min chromatography, 5-95AB, ESI calcd. for C₂₆H₂₃FN₃O [M+H]⁺412.2, found 412.1.

Step 5: To a solution of N-[3-fluoro-4-[(5-methoxy-4-methyl-3-pyridyl)methyl]-2-pyridyl]-1,1-diphenyl-methanimine (400 mg, 972.12 mmol) in DCM (5 mL) was added BBr₃ (730.62 mg, 2.92 mmol, 281.01 mL) at 0° C. The mixture was stirred at 25° C. for 12 hr under N₂. The mixture was added dropwise into H₂O (20 mL) at 0° C. and adjusted to pH=7 with saturated NaHCO₃ solution slowly at 0° C. EtOAc (10 mL) was added and the organic layer was extracted with H₂O (10 mL×2). The aquenous layers was concentrated. The crude was purified by flash column chromatography on silica gel (MeOH in DCM=0-8%) to give 5-[(2-amino-3-fluoro-4-pyridyl)methyl]-4-methyl-pyridin-3-ol (120 mg, 514.49 mmol). ¹H NMR (400 MHz, DMSO-d₆) δ=9.85 (brs, 1H), 8.05 (s, 1H), 7.87 (s, 1H), 7.60 (d, J=5.2 Hz, 1H), 6.16 (t, J=4.8 Hz, 3H), 3.90 (s, 2H), 2.04 (s, 3H). ¹⁹F NMR (376.5 MHz, DMSO-d₆) δ=−145.012.

Step 6: To a solution of 5-[(2-amino-3-fluoro-4-pyridyl)methyl]-4-methyl-pyridin-3-ol (100 mg, 428.74 mmol) in DMF (2 mL) were added 1-bromobut-2-yne (57.02 mg, 428.74 mmol, 37.54 mL) and Cs₂CO₃ (419.08 mg, 1.29 mmol). The mixture was stirred at 25° C. for 1 hr. Water (20 mL) was added and the mixture were extracted with EtOAc (20 ml×2). The organic layer was dried over anhydrous Na₂SO₄, filtered and concentrated. 4-[(5-but-2-ynoxy-4-methyl-3-pyridyl)methyl]-3-fluoro-pyridin-2-amine (90 mg, 315.44 mmol). 1H NMR (400 MHz, CDCl₃) δ=8.28 (brs, 1H), 8.10 (brs, 1H), 7.70 (d, J=5.6 Hz, 1H), 6.24 (t, J=5.2 Hz, 2H), 4.79-4.75 (m, 4H), 3.96 (s, 2H), 2.15 (s, 3H), 1.25 (s, 3H). ¹⁹F NMR (376.5 MHz, CDCl₃) δ=−144.924

Step 7: To a solution of 4-[(5-but-2-ynoxy-4-methyl-3-pyridyl)methyl]-3-fluoro-pyridin-2-amine (50 mg, 175.24 mmol) in MeCN (2 mL) were added N-methylsulfamoyl chloride (113.53 mg, 876.22 mmol) and Py (69.31 mg, 876.22 μmol, 70.72 mL) at 0° C. The mixture was stirred at 0° C. for 1 hr. The mixture was concentrated and purified by Pre-HPLC (column: Phenomenex C 18 80×40 mm×3 mm; mobile phase: [water (NH₃H₂O+NH₄HCO₃)−ACN]; gradient: 20%-50% B over 7 min) and SFC (column: DAICEL CHIRALCEL OD (250 mm×30 mm, 10 mm); mobile phase: [CO₂-EtOH (0.1% NH₃H₂O)]; B %:35% %, isocratic elution mode) to give 4-[(5-but-2-ynoxy-4-methyl-3-pyridyl)methyl]-3-fluoro-N-(methylsulfamoyl)pyridin-2-amine (6.9 mg, 18.23 mmol). ¹H NMR (400 MHz, CDCl₃) δ=8.31 (s, 1H), 8.09 (s, 1H), 7.92 (d, J=5.2 Hz, 1H), 6.56 (t, J=5.2 Hz, 1H), 5.50 (s, 1H), 4.75 (s, 2H), 4.00 (s, 2H), 2.75 (s, 3H), 2.14 (s, 3H), 1.85 (s, 3H). ¹⁹F NMR (376.5 MHz, CDCl₃) δ=−142.486. LCMS Rₜ=0.705 min in 1.5 min chromatography, 5-95AB, ESI calcd. for C₁₇H₂₀FN₄O₃S [M+H]⁺379.1, found 379.0.

### Example 76: 4-[[5-(4-chloro-2-methyl-phenoxy)-4-methyl-3-pyridyl]methyl]-3-fluoro-N-(methylsulfamoyl)pyridin-2-amine

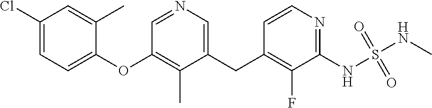 [SMILES: CNS(=O)(=O)Nc1nccc(Cc2cncc(Oc3ccc(Cl)cc3C)c2C)c1F]

### Route

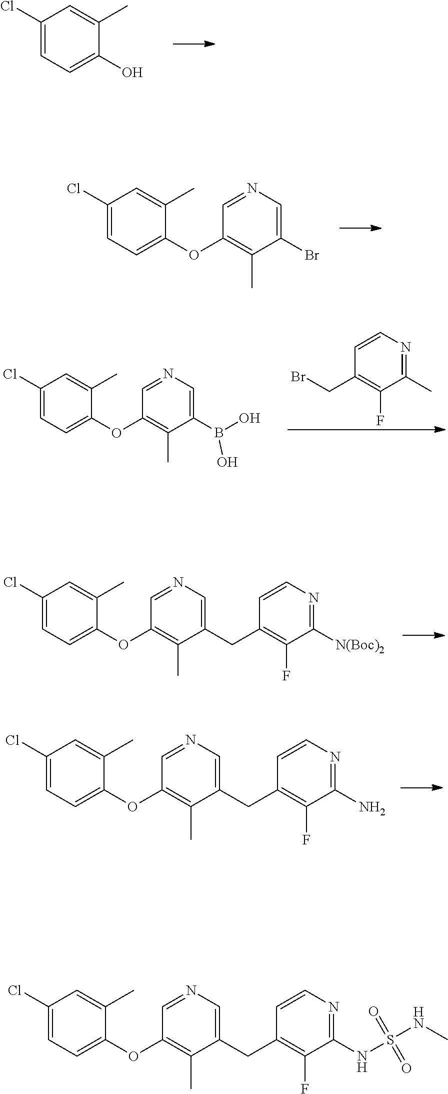

Step 1: To a solution of 4-chloro-2-methyl-phenol (3.00 g, 21.04 mmol) in NMP (30 mL) was added 3,5-dibromo-4-methyl-pyridine (7.92 g, 31.56 mmol) and Cs₂CO₃ (10.28 g, 31.56 mmol). The mixture was stirred at 145° C. for 10 h. The reaction mixture was filtered, then H₂O (100 mL) was added. The aqueous layer was extracted with DCM (100 mL×3). The combined organic layers were washed with brine (100 mL×3), dried over Na₂SO₄, filtered, concentrated. The residue was purified by flash chromatography on silica gel (Ethyl acetate in Petroleum ether=0-30%) to give 3-bromo-5-(4-chloro-2-methyl-phenoxy)-4-methyl-pyridine (2.7 g, 8.64 mmol). ¹H NMR (400 MHz, CDCl₃) δ=8.56 (s, 1H), 8.45 (s, 1H), 7.91 (s, 1H), 7.12 (dd, J=8.8 Hz, 2.0 Hz, 1H), 6.65 (d, J=8.8 Hz), 2.38 (s, 3H), 2.27 (s, 3H). LCMS Rₜ=0.751 min in 1.0 min chromatography, 5-95AB, ESI calcd. for C₁₃H₁₂BrClNO [M+H]⁺312.0, found 312.0.

Step 2: A solution of 3-bromo-5-(4-chloro-2-methyl-phenoxy)-4-methyl-pyridine (0.20 g, 639.82 μmol) in 1,4-dioxane (2.0 mL) was added 4,4,5,5-tetramethyl-2-(4,4,5,5-tetramethyl-1,3,2-dioxaborolan-2-yl)-1,3,2-dioxaborolane (324.95 mg, 1.28 mmol), Pd(dppf)Cl₂ (46.82 mg, 63.98 μmol), potassium acetate (188.38 mg, 1.92 mmol). The mixture was stirred at 100° C. for 12 h under N₂ atmosphere. The reaction mixture was filtered, concentrated, and purified by flash chromatography on silica gel (Ethyl acetate in Petroleum ether=0-30%) to [5-(4-chloro-2-methyl-phenoxy)-4-methyl-3-pyridyl]boronic acid (170 mg, 612.59 μmol). LCMS Rₜ=0.314 min in 0.8 min chromatography, 5-95AB, ESI calcd. for C₁₃H₁₄BClNO₃ [M+H]⁺278.1, found 278.1.

Step 3: A solution of 3-bromo-5-(4-chloro-2-methyl-phenoxy)-4-methyl-pyridine (0.20 g, 639.82 μmol) in 1,4-dioxane (2.0 mL) was added 4,4,5,5-tetramethyl-2-(4,4,5,5-tetramethyl-1,3,2-dioxaborolan-2-yl)-1,3,2-dioxaborolane (324.95 mg, 1.28 mmol), Pd(dppf)Cl₂ (46.82 mg, 63.98 μmol), potassium acetate (188.38 mg, 1.92 mmol). The mixture was stirred at 100° C. for 12 h under N₂ atmosphere. The reaction mixture was filtered, concentrated, and purified by flash chromatography on silica gel (Ethyl acetate in Petroleum ether=0-30%) to [5-(4-chloro-2-methyl-phenoxy)-4-methyl-3-pyridyl]boronic acid (170 mg, 612.59 μmol). LCMS Rₜ=0.314 min in 0.8 min chromatography, 5-95AB, ESI calcd. for C₁₃H₁₄BClNO₃ [M+H]⁺278.1, found 278.1.

Step 4: To a solution of tert-butyl N-tert-butoxycarbonyl-N-[4-[[5-(4-chloro-2-methyl-phenoxy)-4-methyl-3-pyridyl]methyl]-3-fluoro-2-pyridyl]carbamate (179 mg, 320.77 μmol) in 2 mL MeOH was added HCl/MeOH (4 M, 3 mL). The mixture was stirred at 25° C. for 1 hr under N₂. The reaction mixture was concentrated to give 4-[[5-(4-chloro-2-methyl-phenoxy)-4-methyl-3-pyridyl]methyl]-3-fluoro-pyridin-2-amine (107 mg, 263.8 μmol). ¹H NMR (400 MHz, DMSO-d₆) δ=8.38 (s, 1H), 8.10 (s, 1H), 7.75 (d, J=6.4 Hz, 1H), 7.45 (d, J=2.4 Hz, 1H), 7.24 (dd, J=8.4, 2.4 Hz, 1H), 6.78 (d, J=8.4 Hz, 1H), 6.60 (t, J=6.4 Hz, 1H), 4.23 (s, 2H), 2.25 (s, 3H), 2.21 (s, 3H). LCMS Rₜ=0.325 min in 0.8 min chromatography, 5-95AB, ESI calcd. for C₁₉H₁₈ClFN₃O [M+H]⁺358.1, found 358.1.

Step 5: To a solution of 4-[[5-(4-chloro-2-methyl-phenoxy)-4-methyl-3-pyridyl]methyl]-3-fluoro-pyridin-2-amine (80 mg, 202.9 μmol, HCl) in MeCN (5 mL) was added pyridine (114.64 μL, 1.42 mmol) and N-methylsulfamoyl chloride (105.16 mg, 811.63 μmol). The mixture was stirred at 0° C. for 1 h under N₂. The reaction mixture was quenched by addition of 1 mL H₂O at 0° C., and then concentrated under reduced pressure to give a residue. The residue was purified by prep-HPLC (column: Agela DuraShell C18 150×25 mm×5 um; mobile phase: [water(NH₃H₂O)−ACN]; gradient: 18%-48% B over 10 min) to give 4-[[5-(4-chloro-2-methyl-phenoxy)-4-methyl-3-pyridyl]methyl]-3-fluoro-N-(methylsulfamoyl)pyridin-2-amine (5.2 mg, 11.5 μmol). ¹H NMR (400 MHz, CDCl₃) δ=8.14 (s, 1H), 8.02 (d, J=4.4 Hz, 1H), 7.89 (s, 1H), 7.30 (s, 1H), 7.18 (d, J=8.4 Hz, 1H), 6.78-6.72 (m, 1H), 6.66 (t, J=5.2 Hz, 1H), 5.51-5.40 (m, 1H), 4.11 (s, 2H), 2.80 (d, J=5.2 Hz, 3H), 2.37 (s, 3H), 2.22 (s, 3H). ¹⁹F NMR (376.5 MHz, CDCl₃) δ=−142.474 ppm. LCMS Rₜ=2.008 min in 4.0 min chromatography, 5-95AB, ESI calcd. for C₂₀H₂₁ClFN₄O₃S [M+H]⁺451.1, found 451.1.

### Example 77: 4-[[5-(4-chlorophenoxy)-4-methyl-3-pyridyl]oxy]-3-fluoro-N-(methylsulfamoyl)pyridin-2-amine

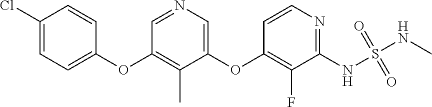 [SMILES: CNS(=O)(=O)Nc1nccc(Oc2cncc(Oc3ccc(Cl)cc3)c2C)c1F]

### Route

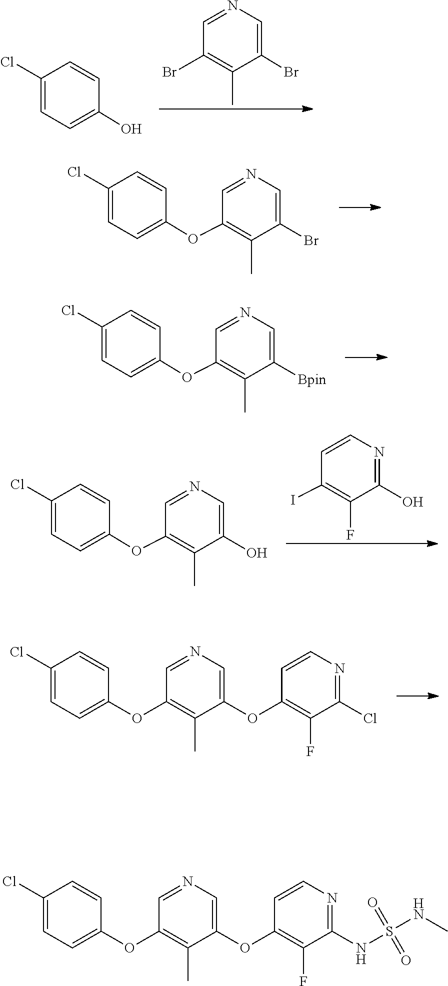

Step 1: A solution of 4-chlorophenol (5 g, 38.89 mmol, 3.83 mL) in NMP (50 mL) were added 3,5-dibromo-4-methyl-pyridine (14.64 g, 58.34 mmol) and Cs₂CO₃ (19.01 g, 58.34 mmol) was stirred at 25° C. for 30 min. Then mixture was stirred at 145° C. for 18 h. The mixture was concentrated o give 3-bromo-5-(4-chlorophenoxy)-4-methyl-pyridine (11.61 g, 38.89 mmol). LCMS Rt=1.76 min in 3 min chromatography, 5-95CD, ESI calcd. for C₁₂H₁₀BrClNO [M+H]⁺298.0, found 297.9.

Step 2:4,4,5,5-tetramethyl-2-(4,4,5,5-tetramethyl-1,3,2-dioxaborolan-2-yl)-1,3,2-dioxaborolane (12.76 g, 50.24 mmol) and Pd(dppf)Cl₂ (612.69 mg, 837.35 μmol) were added under nitrogen to a solution of 3-bromo-5-(4-chlorophenoxy)-4-methyl-pyridine (5 g, 16.75 mmol) in dioxane (50 mL). Then KOAc (4.93 g, 50.24 mmol) was added. The mixture was stirred at 100° C. for 18 hours. The mixture was concentrated. The mixture was purified by 2^{nd} flash chromatography on silica gel (EtOAc in petroleum ether=0-70%) to 3-(4-chlorophenoxy)-4-methyl-5-(4,4,5,5-tetramethyl-1,3,2-dioxaborolan-2-yl)pyridine (4 g, 11.57 mmol). ¹H NMR (400 MHz, CDCl₃) δ=8.70 (s, 1H), 8.25 (s, 1H), 7.27 (d, J=8.0 Hz, 2H), 6.82 (d, J=8.8 Hz, 2H), 2.49 (s, 3H), 1.38 (s, 12H).

Step 3: To a solution of 3-(4-chlorophenoxy)-4-methyl-5-(4,4,5,5-tetramethyl-1,3,2-dioxaborolan-2-yl)pyridine (3 g, 8.68 mmol) in THF (25 mL) and H₂O (25 mL) was added sodium; 3-oxidodioxaborirane; tetrahydrate (1.34 g, 8.68 mmol). The mixture was stirred at 25° C. for 1 h. The mixture was poured into water (20 mL). The aqueous layer was extracted with EtOAc (20 mL×3). The combined organic phase was washed with brine (20 mL), dried over anhydrous Na₂SO₄, filtered and concentrated under reduced pressure. The mixture was purified by flash chromatography on silica gel (EtOAc in petroleum ether=0-70%) to afford 5-(4-chlorophenoxy)-4-methyl-pyridin-3-ol (1.5 g, 6.36 mmol). ¹H NMR (400 MHz, CDCl₃) δ=8.16 (s, 1H), 7.71 (s, 1H), 7.31 (d, J=8.8 Hz, 2H), 6.90 (d, J=8.8 Hz, 2H), 2.22 (s, 3H).

Step 4: To a solution of 5-(4-chlorophenoxy)-4-methyl-pyridin-3-ol (1.4 g, 5.94 mmol) in DMF (5 mL) were added 2-chloro-3-fluoro-4-iodo-pyridine (1.84 g, 7.13 mmol), Cs₂CO₃ (2.90 g, 8.91 mmol), CuI (56.57 mg, 297.03 μmol) and TMHD (547.35 mg, 2.97 mmol, 611.57 μL). The mixture was stirred at 60° C. for 18 h. The mixture was poured into water (20 mL). The aqueous layer was extracted with EtOAc (20 mL×3). The combined organic phase was washed with H₂O (50 mL×3) and brine (50 mL), dried over anhydrous Na₂SO₄, filtered and concentrated under reduced pressure. The mixture was purified by flash chromatography on silica gel (EtOAc in petroleum ether=0-50%) and prep-HPLC (column: Xtimate C18 150×40 mm×10 um; mobile phase: [water(NH₃H₂O+NH₄HCO₃)−ACN]; gradient: 70%-100% B over 8 min) to give 2-chloro-4-[[5-(4-chlorophenoxy)-4-methyl-3-pyridyl]oxy]-3-fluoro-pyridine (800 mg, 2.19 mmol). ¹H NMR (400 MHz, CDCl₃) δ=8.26-8.01 (m, 3H), 7.35 (d, J=9.2 Hz, 2H), 6.95 (d, J=9.2 Hz, 2H), 6.67 (t, J=5.6 Hz, 1H), 2.20 (s, 3H)

Step 5: To a solution of 2-chloro-4-[[5-(4-chlorophenoxy)-4-methyl-3-pyridyl]oxy]-3-fluoro-pyridine (200 mg, 547.67 μmol) in dioxane (5 mL) were added (sulfamoylamino) methane (90.48 mg, 821.50 μmol), Cs₂CO₃ (535.32 mg, 1.64 mmol) and tBuBrett phos pd G3 (46.79 mg, 54.77 μmol) under N₂. The mixture was stirred at 100° C. for 18 h. The mixture was concentrated. The mixture was purified by flash chromatography on silica gel (EtOAc in petroleum ether=0-100%) and prep-HPLC (column: Phenomenex C18 75×30 mm×3 μm; mobile phase: [water(NH₃H₂O+NH₄HCO₃)−ACN]; gradient: 20%-50% B over 8 min) to give 4-[[5-(4-chlorophenoxy)-4-methyl-3-pyridyl]oxy]-3-fluoro-N-(methylsulfamoyl)pyridin-2-amine (20.5 mg, 46.71 μmol). ¹H NMR (400 MHz, CDCl₃) δ=8.21-8.07 (m, 2H), 7.94 (d, J=5.6 Hz, 1H), 7.34 (d, J=8.8 Hz, 2H), 6.94 (d, J=8.8 Hz, 2H), 6.40 (t, J=5.6 Hz, 1H), 5.52 (q, J=3.6 Hz, 1H), 2.79 (d, J=3.6 Hz, 3H), 2.20 (s, 3H). ¹⁹F NMR (376.5 MHz, CDCl₃) δ=−159.362. LCMS Rt=0.898 min in 1.5 min chromatography, 5-95AB, ESI calcd. for C₁₈H₁₇ClFN₄O₄S [M+H]⁺439.1, found 439.0.

### Example 78: {[4-({5-[(4-chlorophenyl) sulfanyl]-4-methylpyridin-3-yl}methyl)-3-fluoropyridin-2-yl]sulfamoyl}(methyl)amine

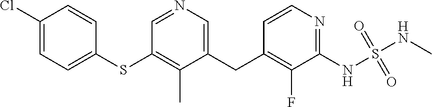 [SMILES: CNS(=O)(=O)Nc1nccc(Cc2cncc(Sc3ccc(Cl)cc3)c2C)c1F]

### Route

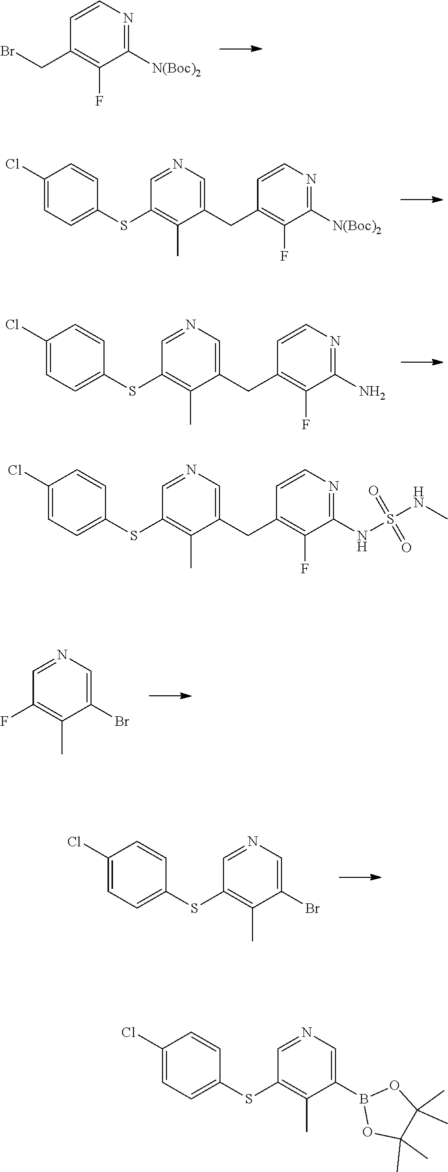

Step 1: To a stirred solution of 4-chlorobenzenethiol (1.14 g, 7.894 mmol) in DMF (20 mL) was added NaH (60 wt %, 473.61 mg, 11.841 mmol, 1.5 equiv) in portions at 0° C. under nitrogen atmosphere. The resulting mixture was stirred for 30 min at 0° C. Then 3-bromo-5-fluoro-4-methylpyridine (1.5 g, 7.894 mmol) was added into the reaction mixture. The resulting reaction mixture was stirred for aditional 16 h at 80° C. Desired product could be detected by LCMS. The reaction was quenched with sat. NH₄Cl (aq.) at 0° C. The resulting mixture was extracted with EA (3×20 mL). The combined organic layers were washed with brine (20 mL), dried over anhydrous Na₂SO₄. After filtration, the filtrate was concentrated under reduced pressure. The residue was purified by silica gel column chromatography, eluted with PE/EA (10:1) to afford 3-bromo-5-[(4-chlorophenyl) sulfanyl]-4-methylpyridine (251 mg). LCMS: [M+1]⁺=314.00. ¹H NMR (400 MHz, Chloroform-d) δ 8.59 (s, 1H), 8.31 (s, 1H), 7.33-7.28 (m, 2H), 7.22-7.16 (m, 2H), 2.50 (s, 3H).

Step 2: A mixture of 3-bromo-5-[(4-chlorophenyl) sulfanyl]-4-methylpyridine (288 mg, 0.915 mmol), 4,4,5,5-tetramethyl-2-(tetramethyl-1,3,2-dioxaborolan-2-yl)-1,3,2-dioxaborolane (348.67 mg, 1.373 mmol, 1.5 equiv), AcOK (269.51 mg, 2.745 mmol, 3 equiv) and Pd(dppf)Cl₂ (66.98 mg, 0.092 mmol, 0.1 equiv) in dioxane (5 mL) was stirred for 16 h at 100° C. under nitrogen atmosphere. Desired product could be detected by LCMS. The resulting mixture was concentrated under reduced pressure. This resulted in 3-((4-chlorophenyl)thio)-4-methyl-5-(4,4,5,5-tetramethyl-1,3,2-dioxaborolan-2-yl)pyridine, LCMS: (ESI, m/z): [M+1]⁺=362.0

Step 3: A mixture of tert-butyl N-[4-(bromomethyl)-3-fluoropyridin-2-yl]-N-(tert-butoxycarbonyl) carbamate (150 mg, 0.370 mmol), 3-[(4-chlorophenyl) sulfanyl]-4-methyl-5-(4,4,5,5-tetramethyl-1,3,2-dioxaborolan-2-yl)pyridine (200.81 mg, 0.555 mmol, 1.5 equiv), Pd(dppf)Cl₂ (27.08 mg, 0.037 mmol, 0.1 equiv) and K₂CO₃ (153.46 mg, 1.110 mmol, 3 equiv) in dioxane (5 mL) and H₂O (0.5 mL) was stirred for 2 h at 80° C. under nitrogen atmosphere. Desired product could be detected by LCMS. The resulting mixture was diluted with water (10 mL) and extracted with EA (3×10 mL). The combined organic layers were washed with brine (10 mL), dried over anhydrous Na₂SO₄. After filtration, the filtrate was concentrated under reduced pressure. The residue was purified by silica gel column chromatography, eluted with PE/EA (2:1) to afford tert-butyl N-(tert-butoxycarbonyl)-N-[4-({5-[(4-chlorophenyl) sulfanyl]-4-methylpyridin-3-yl}methyl)-3-fluoropyridin-2-yl]carbamate (72 mg). LCMS: (ESI, m/z): [M+1]⁺=560.1

Step 4: To a stirred solution of tert-butyl N-(tert-butoxycarbonyl)-N-[4-({5-[(4-chlorophenyl) sulfanyl]-4-methylpyridin-3-yl}methyl)-3-fluoropyridin-2-yl]carbamate (60 mg, 0.107 mmol) in DCM (4 mL) were added TFA (1 mL) dropwise at 0° C. The resulting mixture was stirred for 2 h at room temperature. Desired product could be detected by LCMS. The reaction mixture concentrated under reduced prssure. The residue was purified by reversed-phase flash chromatography with the following conditions: column, C18 silica gel; mobile phase, CN in water (10 mmol/L NH₄HCO₃), 5% to 70% gradient in 30 min; detector, UV 254 nm. This resulted in 4-({5-[(4-chlorophenyl) sulfanyl]-4-methylpyridin-3-yl}methyl)-3-fluoropyridin-2-amine (29 mg). LCMS: (ESI, m/z): [M+1]⁺=360.15. ¹H NMR (300 MHz, Chloroform-d) δ 8.39 (s, 2H), 7.78-7.71 (m, 1H), 7.31 (s, 1H), 7.28 (s, 1H), 7.20-7.12 (m, 2H), 6.34-6.24 (m, 1H), 5.05 (s, 2H), 4.04 (s, 2H), 2.30 (s, 3H) ¹⁹F NMR (282 MHz, Chloroform-d) δ −144.33.

Step 5: To a stirred solution of 4-({5-[(4-chlorophenyl) sulfanyl]-4-methylpyridin-3-yl}methyl)-3-fluoropyridin-2-amine (24 mg, 0.067 mmol) and pyridine (52.76 mg, 0.670 mmol, 10 equiv) in DMA (1 mL) were added N-methylsulfamoyl chloride (10.37 mg, 0.080 mmol, 1.2 equiv) in DMA (1 mL) dropwise at room temperature. The resulting mixture was stirred for 1 h at room temperature. Desired product could be detected by LCMS. The reaction mixture was purified by reversed-phase flash chromatography with the following conditions: column, C18 silica gel; mobile phase, ACN in water (10 mmol/L NH₄HCO₃), 5% to 60% gradient in 30 min; detector, UV 254 nm. This resulted in {[4-({5-[(4-chlorophenyl) sulfanyl]-4-methylpyridin-3-yl}methyl)-3-fluoropyridin-2-yl]sulfamoyl}(methyl)amine (17.4 mg). LCMS: (ESI, m/z): [M+1]⁺=453.10. ¹H NMR (400 MHz, Chloroform-d) δ 8.27 (d, J=6.8 Hz, 2H), 7.98 (d, J=5.1 Hz, 1H), 7.36-7.31 (m, 2H), 7.25-7.21 (m, 2H), 6.58 (t, J=5.1 Hz, 1H), 5.46 (d, J=5.8 Hz, 1H), 4.07 (s, 2H), 2.78 (d, J=4.9 Hz, 3H), 2.35 (s, 3H). ¹⁹F NMR (377 MHz, Chloroform-d) δ −142.48.

### Example 79: N-(4-ethyl-2-fluorophenyl)-5-({3-fluoro-2-[(methylsulfamoyl)amino]pyridin-4-yl}oxy)-4-methylpyridin-3-amine

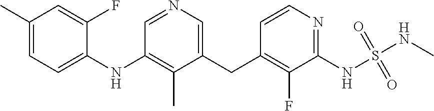

### Route

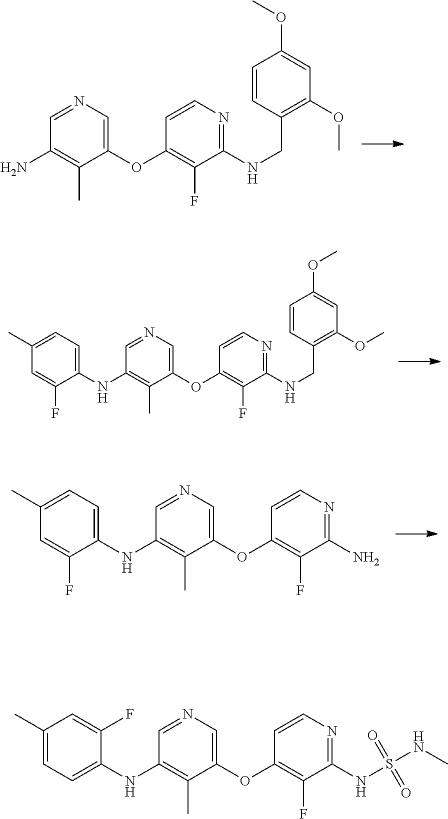

Step 1: To a stirred mixture of 5-[(2-{[(2,4-dimethoxyphenyl)methyl]amino}-3-fluoropyridin-4-yl)oxy]-4-methylpyridin-3-amine (80 mg, 0.208 mmol, example 15) and 1-bromo-4-ethyl-2-fluorobenzene (47.21 mg, 0.250 mmol) in toluene (0.5 mL) were added Cs₂CO₃ (135.61 mg, 0.416 mmol), X-Phos (9.92 mg, 0.021 mmol) and Pd₂(dba)₃ (19.06 mg, 0.021 mmol, 0.1 equiv). The resulting mixture was stirred for 4 h at 110° C. under nitrogen atmosphere. Desired product could be detected by LCMS. The reaction mixture was concentrated under reduced pressure. The residue was purified by reversed-phase flash chromatography with the following conditions: column, C18 silica gel; mobile phase, ACN in water (10 mmol/L NH₄HCO₃), 5% to 70% gradient in 30 min; detector, UV 254 nm. This resulted in 5-[(2-{[(2,4-dimethoxyphenyl)methyl]amino}-3-fluoropyridin-4-yl)oxy]-N-(2-fluoro-4-methylphenyl)-4-methylpyridin-3-amine (53 mg). LCMS: (ESI, m/z): [M+1]⁺=493.20

Step 2: To a stirred mixture of 5-[(2-{[(2,4-dimethoxyphenyl)methyl]amino}-3-fluoropyridin-4-yl)oxy]-N-(2-fluoro-4-methylphenyl)-4-methylpyridin-3-amine (60 mg, 0.122 mmol) in DCM (2 mL) was added TFA (0.5 mL) at 0° C. The resulting mixture was stirred for 1 h at room temperature. Desired product could be detected by LCMS. The reaction mixture was concentrated under reduced pressure. The residue was basified to pH 10 with sat. NaHCO₃ (aq.). The resulting mixture was extracted with EA (3×10 mL). The combined organic layers were washed with brine (10 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 40% gradient in 20 min; detector, UV 254 nm. This resulted in 5-[(2-amino-3-fluoropyridin-4-yl)oxy]-N-(2-fluoro-4-methylphenyl)-4-methylpyridin-3-amine (36 mg). LCMS: (ESI, m/z): [M+1]⁺=343.10. ¹H NMR (400 MHz, Chloroform-d) δ 8.15 (m, 2H), 7.70 (d, J=5.8 Hz, 1H), 7.10-6.81 (m, 3H), 6.10 (t, J=5.7 Hz, 1H), 5.37 (s, 1H), 4.81 (s, 2H), 2.33 (s, 3H), 2.17 (s, 3H).

Step 3: To a stirred solution of 5-[(2-amino-3-fluoropyridin-4-yl)oxy]-N-(2-fluoro-4-methylphenyl)-4-methylpyridin-3-amine (36 mg, 0.105 mmol) and pyridine (83.18 mg, 1.050 mmol, 10 equiv) in DMA (0.5 mL) was added N-methylsulfamoyl chloride (68.12 mg, 0.525 mmol, 5 equiv) in DMA (0.5 mL) dropwise at 0° C. under nitrogen atmosphere. The resulting mixture was stirred for 1 h at room temperature under nitrogen atmosphere. Desired product could be detected by LCMS. The reaction mixture was purified by reversed-phase flash chromatography with the following conditions: column, C18 silica gel; mobile phase, ACN in water (10 mmol/L NH₄HCO₃), 5% to 50% gradient in 30 min; detector, UV 254 nm. This resulted in N-(4-ethyl-2-fluorophenyl)-5-({3-fluoro-2-[(methylsulfamoyl)amino]pyridin-4-yl}oxy)-4-methylpyridin-3-amine (7.7 mg). LCMS: (ESI, m/z): [M+1]⁺=435.95. ¹H NMR (400 MHz, Chloroform-d) δ 8.25 (s, 1H), 7.95 (m, 2H), 7.07 (t, J=8.4 Hz, 1H), 7.00 (m, 1H), 6.93 (d, J=8.2 Hz, 1H), 6.41 (t, J=5.8 Hz, 1H), 5.45 (d, J=22.0 Hz, 2H), 2.81 (d, J=4.4 Hz, 3H), 2.35 (s, 3H), 2.21 (s, 3H). ¹⁹F NMR (377 MHz, DMSO-d₆) δ −124.511, −156.618.

### Example 80: N-(4-ethyl-2-fluorophenyl)-5-({3-fluoro-2-[(methylsulfamoyl)amino]pyridin-4-yl}oxy)-4-methylpyridin-3-amine

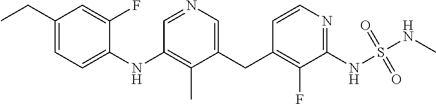 [SMILES: CCc1ccc(Nc2cncc(Cc3ccnc(NS(=O)(=O)NC)c3F)c2C)c(F)c1]

### Route

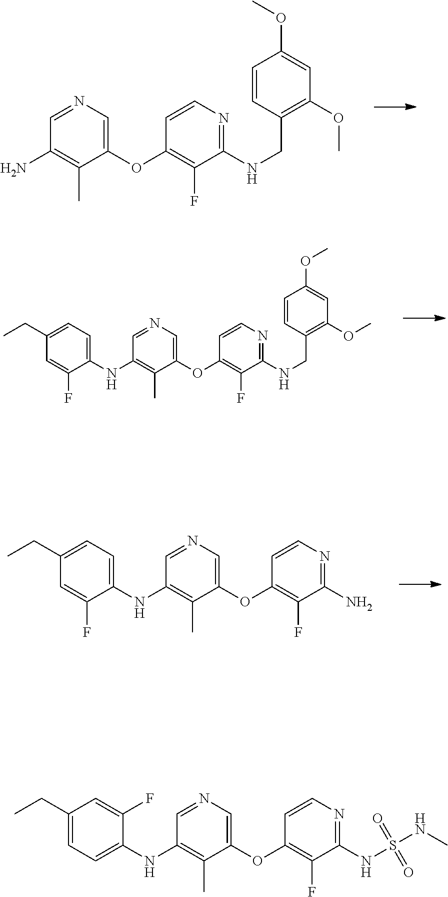

Step 1: To a solution of 5-[(2-{[(2,4-dimethoxyphenyl)methyl]amino}-3-fluoropyridin-4-yl)oxy]-4-methylpyridin-3-amine (120 mg, 0.312 mmol, example 15) and 1-bromo-4-ethyl-2-fluorobenzene (76.06 mg, 0.374 mmol) in Toluene (5 mL) were added Cs₂CO₃ (203.42 mg, 0.624 mmol), X-Phos (14.88 mg, 0.031 mmol) and Pd₂(dba)₃ (28.59 mg, 0.031 mmol). The resulting mixture was stirred for 4 h at 110° C. under nitrogen atmosphere. Desired product could be detected by LCMS. The resulting mixture was concentrated under reduced pressure. The residue was purified by reversed-phase flash chromatography with the following conditions: column, C18 silica gel; mobile phase, ACN in water (10 mmol/L NH₄HCO₃), 5% to 70% gradient in 30 min; detector, UV 254 nm. This resulted in 5-[(2-{[(2,4-dimethoxyphenyl)methyl]amino}-3-fluoropyridin-4-yl)oxy]-N-(4-ethyl-2-fluorophenyl)-4-methylpyridin-3-amine (110 mg). LCMS: (ESI, m/z): [M+1]⁺=507.45

Step 2: To a stirred solution of 5-[(2-{[(2,4-dimethoxyphenyl)methyl]amino}-3-fluoropyridin-4-yl)oxy]-N-(4-ethyl-2-fluorophenyl)-4-methylpyridin-3-amine (90 mg, 0.178 mmol) and DCM (2 mL) was added TFA (0.5 mL) at 0° C. The resulting mixture was stirred for 1 h at room temperature. Desired product could be detected by LCMS. The reaction mixture was concentrated under reduced pressure. The residue was basified to pH 10 with sat. NaHCO₃ (aq.). The resulting mixture was extracted with EA (3×20 mL). The combined organic layers were washed with brine (20 mL), dried over anhydrous Na₂SO₄. After filtration, the filtrate was concentrated under reduced pressure. The residue was purified by reversed-phase flash chromatography with the following conditions: column, C18 silica gel; mobile phase, ACN in water (10 mmol/L NH₄HCO₃), 5% to 40% gradient in 20 min; detector, UV 254 nm. This resulted in 5-[(2-amino-3-fluoropyridin-4-yl)oxy]-N-(4-ethyl-2-fluorophenyl)-4-methylpyridin-3-amine (60 mg). LCMS: (ESI, m/z): [M+1]⁺=357.0

Step 3: To a solution of 5-[(2-amino-3-fluoropyridin-4-yl)oxy]-N-(4-ethyl-2-fluorophenyl)-4-methylpyridin-3-amine (55 mg, 0.154 mmol) and pyridine (122.08 mg, 1.540 mmol, 10 equiv) in DMA (0.5 mL) was added and N-methylsulfamoyl chloride (99.98 mg, 0.770 mmol, 5 equiv) in DMA (0.5 mL) dropwise at 0° C. The resulting mixture was stirred for 2 h at room temperature under nitrogen atmosphere. Desired product could be detected by LCMS. The reaction mixture was purified by reversed-phase flash chromatography with the following conditions: column, C18 silica gel; mobile phase, ACN in Water (10 mmol/L NH₄HCO₃), 5% to 50% gradient in 20 min; detector, UV 254 nm. This resulted in N-(4-ethyl-2-fluorophenyl)-5-({3-fluoro-2-[(methylsulfamoyl)amino]pyridin-4-yl}oxy)-4-methylpyridin-3-amine (39 mg). LCMS: (ESI, m/z): [M+1]⁺=450.15. ¹H NMR (400 MHz, DMSO-d₆) δ 10.51 (s, 1H), 7.96 (d, J=6.2 Hz, 2H), 7.91 (d, J=2.1 Hz, 1H), 7.57 (s, 1H), 7.17-7.09 (m, 1H), 7.06-6.95 (m, 2H), 6.47 (t, J=5.6 Hz, 1H), 2.63-2.54 (m, 2H), 2.54 (s, 3H), 2.07 (s, 3H), 1.19 (t, J=7.6 Hz, 3H). ¹⁹F NMR (377 MHz, DMSO-d₆) δ −124.421, −156.639.

### Example 81: 4-[[5-(4-chloro-2-fluoro-phenoxy)-4-methyl-3-pyridyl]methyl]-N-(ethylsulfamoyl)-3-fluoro-pyridin-2-amine

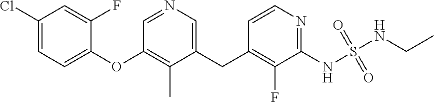 [SMILES: CCNS(=O)(=O)Nc1nccc(Cc2cncc(Oc3ccc(Cl)cc3F)c2C)c1F]

### Route

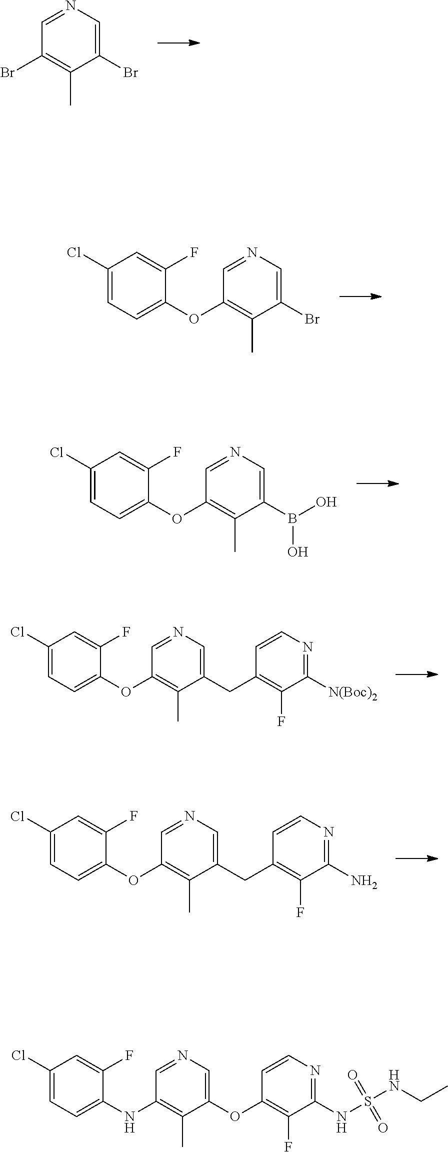

Step 1: To a solution of 4-chloro-2-fluoro-phenol (7.01 g, 47.8 mmol) in NMP (50 mL) and 3,5-dibromo-4-methyl-pyridine (10.0 g, 39.7 mmol) was added Cs₂CO₃ (19.5 g, 59.8 mmol). The mixture was stirred at 145° C. for 18 h. After cooling to room temperature, the mixture was poured into water (50 mL). The aqueous layer was extracted with EtOAc (50 mL×3). The combined organic phase was washed with H₂O (100 mL×3) and brine (20 mL), dried over anhydrous Na₂SO₄, filtered, concentrated and purified by prep-HPLC (column: Phenomenex luna C18 (250×70 mm, 10 um); mobile phase: [water(NH₄HCO₃)−ACN]; gradient: 50%-80% B) to give 3-bromo-5-(4-chloro-2-fluoro-phenoxy)-4-methyl-pyridine (800 mg, 2.53 mmol). ¹H NMR (400 MHz, CDCl₃) δ=8.48 (s, 1H), 7.99 (s, 1H), 7.26-7.22 (m, 1H), 7.13-7.09 (m, 1H), 6.91 (t, J=8.8 Hz, 1H), 2.42 (s, 3H). ¹⁹F NMR (376.5 MHz CDCl₃) δ=−127.908 ppm. LCMS Rₜ=0.512 min in 0.8 min chromatography, 5-95AB, ESI calcd. for C₁₂H₉BrClNOF [M+H]⁺315.9 found 315.9.

Step 2: A mixture of 3-bromo-5-(4-chloro-2-fluoro-phenoxy)-4-methyl-pyridine (1.00 g, 3.16 mmol), 4,4,5,5-tetramethyl-2-(4,4,5,5-tetramethyl-1,3,2-dioxaborolan-2-yl)-1,3,2-dioxaborolane (1.60 g, 6.32 mmol), Pd(dppf)Cl₂ (231 mg, 316 μmol), KOAc (930 mg, 9.48 mmol) in dioxane (10 mL) was stirred at 80° C. for 12 h under N₂. After cooling to room temperature, the reaction mixture was poured into H₂O (5 mL) and extracted with EtOAc (5 mL×3). The combined organic layers were washed with brine (5 mL), dried over anhydrous Na₂SO₄, filtered and concentrated. The crude product [5-(4-chloro-2-fluoro-phenoxy)-4-methyl-3-pyridyl]boronic acid (889.19 mg, 3.16 mmol, 100.00%). LCMS Rₜ=0.303 min in 0.8 min chromatography, 5-95AB, ESI calcd. for C₁₂H₁₁BClFNO₃ [M+H]⁺282.1, 281.9.

Step 3: A mixture of tert-butyl N-[4-(bromomethyl)-3-fluoro-2-pyridyl]-N-tert-butoxycarbonyl-carbamate (892 mg, 2.20 mmol), [5-(4-chloro-2-fluoro-phenoxy)-4-methyl-3-pyridyl]boronic acid (774 mg, 2.75 mmol), Pd(PPh₃)₄ (159 mg, 138 μmol), Na₂CO₃ (874 mg, 8.25 mmol) in a mixed solvent of H₂O (0.1 mL), EtOH (0.5 mL) and toluene (1 mL) was stirred at 80° C. for 1 h under N₂. After cooling to room temperature, the reaction mixture was filtered, concentrated and purified by flash chromatography on silica gel (EtOAc in Petroleum ether=0-20%) to give to give tert-butyl N-tert-butoxycarbonyl-N-[4-[[5-(4-chloro-2-fluoro-phenoxy)-4-methyl-3-pyridyl]methyl]-3-fluoro-2-pyridyl]carbamate (700 mg, 1.25 mmol). ¹H NMR (400 MHz CDCl₃) δ=8.20 (s, 2H), 8.03 (s, 1H), 7.22 (d, J=2.4 Hz, 1H), 7.11 (d, J=8.8 Hz, 2H), 6.94-6.91 (m, 2H), 4.11 (s, 2H), 2.22 (s, 3H), 1.42 (s, 18H). ¹⁹F NMR (376.5 MHz CDCl₃) δ=−128.006, −130.940 ppm. LCMS Rₜ=0.515 min in 0.8 min chromatography, 5-95AB, ESI calcd. for C₂₈H₃₁ClF₂N₃O₅ [M+H]⁺562.2, found 562.1.

Step 4: To a solution of tert-butyl N-tert-butoxycarbonyl-N-[4-[[5-(4-chloro-2-fluoro-phenoxy)-4-methyl-3-pyridyl]methyl]-3-fluoro-2-pyridyl]carbamate (700 mg, 1.25 mmol) in MeOH (1 mL) was added HCl/MeOH (1.5 mL). The mixture was stirred at 25° C. for 36 h. The reaction mixture was filtered and concentrated to give 4-[[5-(4-chloro-2-fluoro-phenoxy)-4-methyl-3-pyridyl]methyl]-3-fluoro-pyridin-2-amine (100 mg, 276 μmol) ¹H NMR (400 MHz, CDCl₃) δ=8.21 (s, 1H), 8.04 (s, 1H), 7.73 (d, J=5.2 Hz, 1H), 7.24-7.21 (m, 1H), 7.10-7.07 (m, 1H), 6.87 (t, J=8.8 Hz, 1H), 6.28 (t, J=5.2 Hz, 1H), 4.81 (br s, 2H), 4.01 (s, 2H), 2.22 (s, 3H). ¹⁹F NMR (376.5 MHz, CDCl₃) δ=−128.351, −144.847 ppm. LCMS Rₜ=0.305 min in 0.8 min chromatography, 5-95AB, ESI calcd. for C₁₈H₁₅ClF₂N₃O [M+H]⁺361.1, found 361.9.

Step 5: To a solution of N-ethylsulfamoyl chloride (39.7 mg, 276 μmol) in CH₃CN (10 mL) was added pyridine (76.5 mg, 967 μmol) and 4-[[5-(4-chloro-2-fluoro-phenoxy)-4-methyl-3-pyridyl]methyl]-3-fluoro-pyridin-2-amine (50.0 mg, 138 μmol) at 0° C. The mixture was stirred at 25° C. for 1 h. The reaction mixture was filtered, concentrated and purified by prep-HPLC (column: Phenomenex C18 150×25 mm×10 um; mobile phase: [water(NH₃H₂O)−ACN]; gradient: 14%-44% B over 10 min) to give 4-[[5-(4-chloro-2-fluoro-phenoxy)-4-methyl-3-pyridyl]methyl]-N-(ethylsulfamoyl)-3-fluoro-pyridin-2-amine (26.7 mg, 56.9 μmol). ¹H NMR (400 MHz, CDCl₃) δ=8.19 (s, 1H), 8.05-7.97 (m, 2H), 7.24 (d, J=2.4 Hz, 1H), 7.15-7.12 (m, 1H), 6.95 (t, J=8.8 Hz, 1H), 6.61 (t, J=5.2 Hz, 1H), 5.44 (br s, 1H), 4.08 (s, 2H), 3.18-3.11 (m, 2H), 2.29 (s, 3H), 1.21 (t, J=7.2 Hz, 3H) ¹⁹F NMR (376.5 MHz, CDCl₃) δ=−127.758, −142.769 ppm. LCMS Rₜ=0.441 min in 1.0 min chromatography, 5-95AB, ESI calcd. for C₂₀H₂₀IF₂N₄O₃S [M+H]⁺469.1, found 469.2.

### Example 82: 4-[[5-[(4-chlorophenyl) methoxy]-4-methyl-3-pyridyl]methyl]-3-fluoro-N-(methylsulfamoyl)pyridin-2-amine

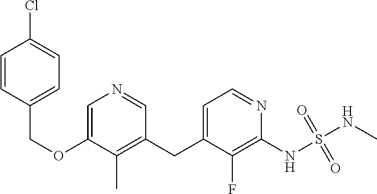 [SMILES: CNS(=O)(=O)Nc1nccc(Cc2cncc(OCc3ccc(Cl)cc3)c2C)c1F]

### Route

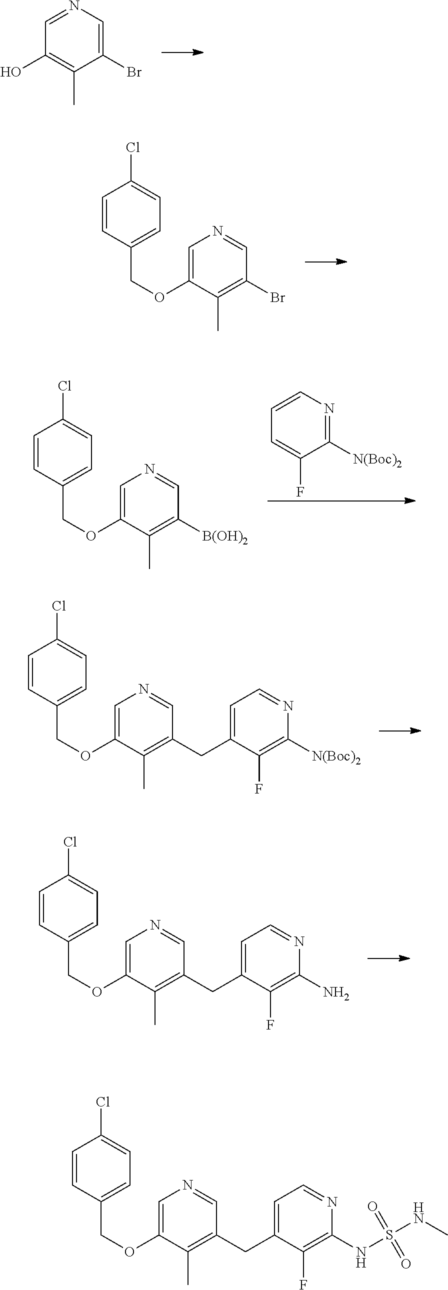

Step 1: To a solution of 5-bromo-4-methyl-pyridin-3-ol (2 g, 10.64 mmol, example 30) in DMF (18 mL) was added 1-(bromomethyl)-4-chloro-benzene (2.62 g, 12.76 mmol) and Cs₂CO₃ (10.40 g, 31.91 mmol). The mixture was stirred at 80° C. for 12 h. Water (50 mL) was added. The mixture was extracted with EtOAc (40 mL×3). The aqueous layer was washed with H₂O (50 mL×5). The combined organic layers were washed with brine (30 mL), dried over anhydrous Na₂SO₄, filtered and concentrated. The crude product was purified by flash chromatography on silica gel (DCM in Petroleum ether=0-15%) to give 3-bromo-5-[(4-chlorophenyl) methoxy]-4-methyl-pyridine (1.08 g, 3.46 mmol). ¹H NMR (400 MHz, CDCl₃) δ=8.34 (s, 1H), 8.12 (s, 1H), 7.42-7.30 (m, 4H), 5.12 (s, 2H), 2.36 (s, 3H).

Step 2-3: To a solution of 3-bromo-5-[(4-chlorophenyl) methoxy]-4-methyl-pyridine (660 mg, 2.11 mmol) in dioxane (9 mL) were added 4,4,5,5-tetramethyl-2-(4,4,5,5-tetramethyl-1,3,2-dioxaborolan-2-yl)-1,3,2-dioxaborolane (697.01 mg, 2.74 mmol), KOAc (621.65 mg, 6.33 mmol) and Pd(dppf)Cl₂ (154.49 mg, 211.14 μmol). The mixture was stirred at 110° C. for 10 h. To a solution of 3-[(4-chlorophenyl) methoxy]-4-methyl-5-(4,4,5,5-tetramethyl-1,3,2-dioxaborolan-2-yl)pyridine (759.37 mg, 2.11 mmol) in toluene (10 mL) and H₂O (2 mL) was added tert-butyl N-[4-(bromomethyl)-3-fluoro-2-pyridyl]-N-tert-butoxycarbonyl-carbamate (1.03 g, 2.53 mmol), Pd(dppf)Cl₂·CH₂Cl₂ (155.18 mg, 190.03 μmol) and Cs₂CO₃ (1.72 g, 5.28 mmol). The mixture was stirred at 100° C. for 2 h. Water (50 mL) was added. The mixture were extracted with EtOAc (40 mL×3). The combined organic layers were washed with brine (30 mL), dried over anhydrous Na₂SO₄, filtered and concentrated. The crude product was purified by flash chromatography on silica gel (EtOAc in Petroleum=0-70%) to give tert-butyl N-tert-butoxycarbonyl-N-[4-[[5-[(4-chlorophenyl) methoxy]-4-methyl-3-pyridyl]methyl]-3-fluoro-2-pyridyl]carbamate (450 mg, 806.39 μmol). ¹H NMR (400 MHz, CDCl₃) δ=8.26-8.04 (m, 2H), 7.62-7.69 (m, 1H), 7.37 (s, 4H), 6.90-6.84 (m, 1H), 5.14 (s, 2H), 4.15-4.05 (m, 2H), 2.14 (s, 3H), 1.41 (s, 18H). ¹⁹F NMR (376.5 MHz, CDCl₃) δ=−131.093.

Step 4: To a solution of tert-butyl N-tert-butoxycarbonyl-N-[4-[[5-[(4-chlorophenyl) methoxy]-4-methyl-3-pyridyl]methyl]-3-fluoro-2-pyridyl]carbamate (450 mg, 806.39 μmol) in MeOH (1 mL) was added HCl/MeOH (4 M, 5 mL, 20.00 mmol). The mixture was stirred at 25° C. for 12 h. NH₃/MeOH (10 mL×3) was added. The mixture was concentrated. The crude product was purified by flash chromatography on silica gel (EtOAc in Petroleum ether=0-92%) to give 4-[[5-[(4-chlorophenyl) methoxy]-4-methyl-3-pyridyl]methyl]-3-fluoro-pyridin-2-amine (180 mg, 503.06 μmol). ¹H NMR (400 MHz, CDCl₃) δ=8.36-8.01 (m, 2H), 7.70 (s, 1H), 7.37 (s, 4H), 6.27 (s, 1H), 5.27-4.82 (m, 4H), 4.00 (s, 2H), 2.40-2.23 (m, 3H). ¹⁹F NMR (376.5 MHz, CDCl₃) δ=−144.273.

Step 5: To a solution of 3-bromo-5-[(4-chlorophenyl) methoxy]-4-methyl-pyridine (660 mg, 2.11 mmol) in dioxane (9 mL) were added 4,4,5,5-tetramethyl-2-(4,4,5,5-tetramethyl-1,3,2-dioxaborolan-2-yl)-1,3,2-dioxaborolane (697.01 mg, 2.74 mmol), KOAc (621.65 mg, 6.33 mmol) and Pd(dppf)Cl₂ (154.49 mg, 211.14 μmol). The mixture was stirred at 110° C. for 10 h. To a solution of 3-[(4-chlorophenyl) methoxy]-4-methyl-5-(4,4,5,5-tetramethyl-1,3,2-dioxaborolan-2-yl)pyridine (759.37 mg, 2.11 mmol) in toluene (10 mL) and H₂O (2 mL) was added tert-butyl N-[4-(bromomethyl)-3-fluoro-2-pyridyl]-N-tert-butoxycarbonyl-carbamate (1.03 g, 2.53 mmol), Pd(dppf)Cl₂·CH₂Cl₂ (155.18 mg, 190.03 μmol) and Cs₂CO₃ (1.72 g, 5.28 mmol). The mixture was stirred at 100° C. for 2 h. Water (50 mL) was added. The mixture were extracted with EtOAc (40 mL×3). The combined organic layers were washed with brine (30 mL), dried over anhydrous Na₂SO₄, filtered and concentrated. The crude product was purified by flash chromatography on silica gel (EtOAc in Petroleum=0-70%) to give tert-butyl N-tert-butoxycarbonyl-N-[4-[[5-[(4-chlorophenyl) methoxy]-4-methyl-3-pyridyl]methyl]-3-fluoro-2-pyridyl]carbamate (450 mg, 806.39 μmol). ¹H NMR (400 MHz, CDCl₃) δ=8.26-8.04 (m, 2H), 7.62-7.69 (m, 1H), 7.37 (s, 4H), 6.90-6.84 (m, 1H), 5.14 (s, 2H), 4.15-4.05 (m, 2H), 2.14 (s, 3H), 1.41 (s, 18H). ¹⁹F NMR (376.5 MHz, CDCl₃) δ=−131.093

### Example 83: 4-[[5-(4-chlorophenoxy)-4-ethyl-3-pyridyl]methyl]-3-fluoro-N-(methylsulfamoyl)pyridin-2-amine

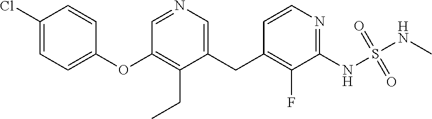 [SMILES: CCc1c(Cc2ccnc(NS(=O)(=O)NC)c2F)cncc1Oc1ccc(Cl)cc1]

### Route

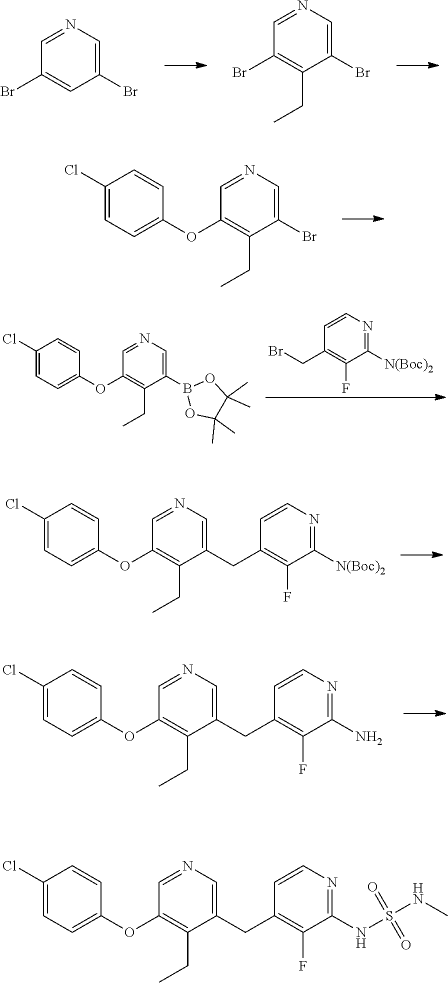

Step 1: To a solution of LDA (2 M, 11.61 mL) in THF (30 mL) and then the solution of 3,5-dibromopyridine (5 g, 21.11 mmol) in THF (20 mL) was added. The solution was stirred at −60° C. for 1 h. After EtI (3.29 g, 21.11 mmol, 1.69 mL) was added. The mixture was stirred at 25° C. for 17 h. The mixture was poured into water (50 mL). The aqueous layer was extracted with EtOAc (50 mL×3). The combined organic phase was washed with brine (50 mL), dried over anhydrous Na₂SO₄, filtered and concentrated under reduced pressure. The mixture was purified by flash chromatography on silica gel (EtOAc in petroleum ether=0-0%) to afford 3,5-dibromo-4-ethyl-pyridine (5 g, 18.87 mmol). ¹H NMR (400 MHz, CDCl₃) δ=8.55 (s, 2H), 2.98 (q, J=7.6 Hz, 2H), 1.18 (t, J=7.6 Hz, 3H).

Step 2: To a solution of 4-chlorophenol (2.91 g, 22.65 mmol, 2.23 mL) in NMP (50 mL) were added 3,5-dibromo-4-ethyl-pyridine (5 g, 18.87 mmol) and Cs₂CO₃ (12.30 g, 37.74 mmol). After stirring at 25° C. for 30 min. Then mixture was stirred at 145° C. for 18 h. The mixture was poured into water (50 mL). The aqueous layer was extracted with EtOAc (50 mL×3). The combined organic phase was washed with H₂O (50 mL×3), brine (50 mL), dried over anhydrous Na₂SO₄, filtered and concentrated under reduced pressure. The mixture was purified by flash chromatography on silica gel (EtOAc in petroleum ether=0%) and prep-HPLC (column: Xtimate C18 150×40 mm×10 μm; mobile phase: [water(NH₃H₂O+NH₄HCO₃)−ACN]; gradient: 70%-100% B over 8 min) to give 3-bromo-5-(4-chlorophenoxy)-4-ethyl-pyridine (2.2 g, 7.04 mmol). ¹H NMR (400 MHz, CDCl₃) δ=8.48 (s, 1H), 8.07 (s, 1H), 7.31 (d, J=9.2 Hz, 2H), 6.89 (d, J=8.8 Hz, 2H), 2.83 (q, J=7.6 Hz, 2H), 1.16 (t, J=7.6 Hz, 3H).

Step 3:3-bromo-5-(4-chlorophenoxy)-4-ethyl-pyridine (2 g, 6.40 mmol) and Pd(dppf)Cl₂ (234.08 mg, 319.91 μmol) were added to a solution of 4,4,5,5-tetramethyl-2-(4,4,5,5-tetramethyl-1,3,2-dioxaborolan-2-yl)-1,3,2-dioxaborolane (4.87 g, 19.19 mmol) in dioxane (20 mL) under N₂. The medium was degased for 5 minutes under N₂ before adding KOAc (1.88 g, 19.19 mmol). The reaction medium was stirred at 100° C. for 18 h. The mixture was concentrated. The mixture was purified by flash chromatography on silica gel (EtOAc in petroleum ether=0-20%) to afford 3-(4-chlorophenoxy)-4-ethyl-5-(4,4,5,5-tetramethyl-1,3,2-dioxaborolan-2-yl)pyridine (1 g, 2.78 mmol). LCMS Rt=1.59 min in 3 min chromatography, 5-95CD, ESI calcd. for C₁₉H₂₄BClNO₃ [M+H]⁺360.2, found 360.1.

Step 4: To a solution of tert-butyl N-[4-(bromomethyl)-3-fluoro-2-pyridyl]-N-tert-butoxycarbonyl-carbamate (1.24 g, 3.06 mmol) in toluene (10 mL), EtOH (2.5 mL) and H₂O (1 mL) were added Na₂CO₃ (884.10 mg, 8.34 mmol), Pd(PPh₃)₄ (321.30 mg, 278.05 μmol) and 3-(4-chlorophenoxy)-4-ethyl-5-(4,4,5,5-tetramethyl-1,3,2-dioxaborolan-2-yl)pyridine (1 g, 2.78 mmol). The mixture was stirred at 100° C. for 12 h. The mixture was concentrated. The mixture was purified by flash chromatography on silica gel (EtOAc in petroleum ether=0-70%) to afford tert-butyl N-tert-butoxycarbonyl-N-[4-[[5-(4-chlorophenoxy)-4-ethyl-3-pyridyl]methyl]-3-fluoro-2-pyridyl]carbamate (1 g, 1.79 mmol). ¹H NMR (400 MHz, CDCl₃) δ=8.23-8.18 (m, 1H), 8.16 (s, 1H), 8.11-8.09 (m, 1H), 7.33-7.27 (m, 2H), 6.97-6.92 (m, 1H), 6.91-6.85 (m, 2H), 4.12 (s, 3H), 2.63 (q, J=7.6 Hz, 2H), 1.23 (s, 18H), 1.06 (t, J=7.6 Hz, 3H).

Step 5: A solution of tert-butyl N-tert-butoxycarbonyl-N-[4-[[5-(4-chlorophenoxy)-4-ethyl-3-pyridyl]methyl]-3-fluoro-2-pyridyl]carbamate (1 g, 1.79 mmol) in HCl/MeOH (10 mL, 4 M) was stirred at 20° C. for 18 h. The mixture was concentrated to give 4-[[5-(4-chlorophenoxy)-4-ethyl-3-pyridyl]methyl]-3-fluoro-pyridin-2-amine (837.2 mg, 1.79 mmol). LCMS Rt=2.090 min in 3 min chromatography, 10-80CD, ESI calcd. for C₁₉H₁₈ClFN₃O [M+H]⁺358.1, found 358.2.

Step 6: To a solution of 4-[[5-(4-chlorophenoxy)-4-ethyl-3-pyridyl]methyl]-3-fluoro-pyridin-2-amine (300 mg, 642.14 μmol, 3HCl) in MeCN (3 mL) were added Py (507.93 mg, 6.42 mmol, 518.29 μL) and methylsulfamoyl chloride (166.40 mg, 1.28 mmol) at 0° C. The mixture was stirred at 0° C. for 2 h. The mixture was concentrated. The crude was purified by prep-HPLC (column: Phenomenex C18 75×30 mm×3 um; mobile phase: [water(NH₃H₂O+NH₄HCO₃)−ACN]; gradient: 27%-57% B over 8 min) to give 4-[[5-(4-chlorophenoxy)-4-ethyl-3-pyridyl]methyl]-3-fluoro-N-(methylsulfamoyl)pyridin-2-amine (65 mg, 144.15 μmol). ¹H NMR (400 MHz, CDCl₃) δ=8.19 (s, 1H), 8.12 (s, 1H), 7.97 (d, J=4.8 Hz, 1H), 7.43-7.27 (m, 3H), 6.90 (d, J=9.2 Hz, 2H), 6.63 (t, J=4.8 Hz, 1H), 5.49 (br s, 1H), 4.07 (s, 2H), 2.77 (d, J=5.2 Hz, 3H), 2.63 (q, J=8.4 Hz, 2H), 1.07 (t, J=7.6 Hz, 3H). ¹⁹F NMR (376.5 MHz, DMSO-d₆) δ=−142.798. LCMS Rt=0.825 min in 1.5 min chromatography, 5-95AB, ESI calcd. for C₂₀H₂₁ClFN₄O₃S [M+H]⁺451.1, found 451.1.

### Example 84: 4-[[5-(2-chloro-4-methyl-phenoxy)-4-methyl-3-pyridyl]oxy]-3-fluoro-N-(methylsulfamoyl)pyridin-2-amine

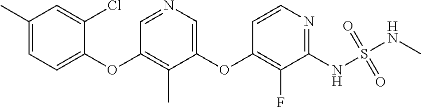

### Route

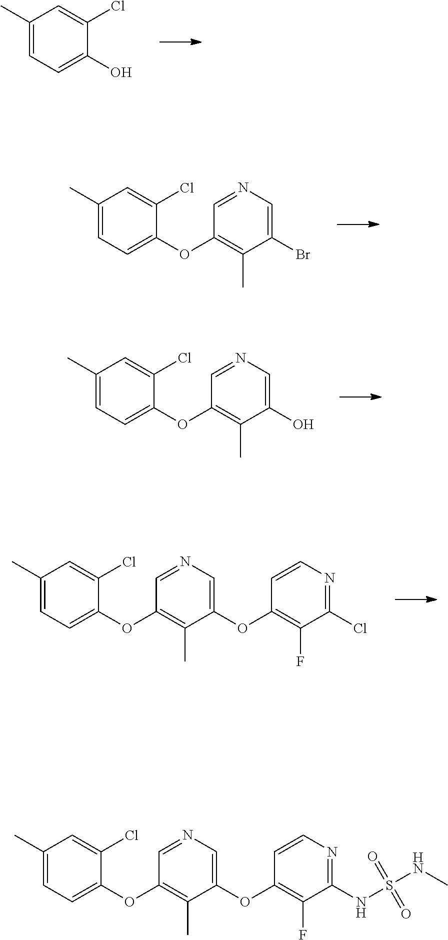

Step 1: To a solution of 3,5-dibromo-4-methyl-pyridine (13.20 g, 52.60 mmol) and 2-chloro-4-methyl-phenol (5.00 g, 35.07 mmol) in NMP (50 mL) were added Cs₂CO₃ (34.28 g, 105.20 mmol). The mixture was stirred at 100° C. for 12 hr. The reaction was concentrated. Water (50 mL) was added and the aqueous was extracted with DCM (2×50 mL). The combined organic layers were dried over anhydrous Na₂SO₄, filtered and concentrated. The residue was purified by flash chromatography on silica gel (Ethyl acetate in Petroleum ether=0-5%) to afford 3-bromo-5-(2-chloro-4-methyl-phenoxy)-4-methyl-pyridine (8.8 g, 28.15 mmol). LCMS Rₜ=1.046 min in 1.5 min chromatography, 5-95AB, ESI calcd. for C₁₃H₁₂BrClNO [M+H]⁺312.0, found 311.8.

Step 2: To a solution of 3-bromo-5-(2-chloro-4-methyl-phenoxy)-4-methyl-pyridine (3.8 g, 12.16 mmol) and 4,4,5,5-tetramethyl-2-(4,4,5,5-tetramethyl-1,3,2-dioxaborolan-2-yl)-1,3,2-dioxaborolane (6.17 g, 24.31 mmol) in dioxane (40 mL) were added KOAc (3.58 g, 36.47 mmol) and Pd(dppf)Cl₂ (444.75 mg, 607.83 μmol). The mixture was stirred at 100° C. for 18 hr. The reaction was concentrated to give 3-(2-chloro-4-methyl-phenoxy)-4-methyl-5-(4,4,5,5-tetramethyl-1,3,2-dioxaborolan-2-yl)pyridine (4.37 g, 12.15 mmol, 100.00% yield) as black brown oil. To a solution of 3-(2-chloro-4-methyl-phenoxy)-4-methyl-5-(4,4,5,5-tetramethyl-1,3,2-dioxaborolan-2-yl)pyridine (4.37 g, 12.15 mmol) in THF (20 mL) and H₂O (20 mL) was added sodium; 3-oxidodioxaborirane; tetrahydrate (3.74 g, 24.30 mmol, 4.67 mL). The mixture was stirred at 60° C. for 2 hr. The reaction was concentrated. Water (20 mL) was added and the aqueous was extracted with DCM (2×20 mL). The combined organic layers were dried over anhydrous Na₂SO₄, filtered and concentrated to give 5-(2-chloro-4-methyl-phenoxy)-4-methyl-pyridin-3-ol (1.3 g, 5.21 mmol). LCMS Rₜ=0.873 min in 1.5 min chromatography, 5-95AB, ESI calcd. for C₁₃H₁₃ClNO₂ [M+H]⁺250.1, found 249.9.

Step 3: To a solution of 5-(2-chloro-4-methyl-phenoxy)-4-methyl-pyridin-3-ol (1.3 g, 5.21 mmol) and 2-chloro-3-fluoro-4-iodo-pyridine (2.68 g, 10.41 mmol) in DMF (10 mL) were added CuI (49.58 mg, 260.32 μmol), TMHD (479.71 mg, 2.60 mmol, 535.98 μL) and Cs₂CO₃ (2.54 g, 7.81 mmol). The mixture was stirred at 60° C. for 18 hr. The reaction was concentrated. Water (10 mL) was added and the aqueous was extracted with Ethyl acetate (10 mL). The combined organic layers were dried over anhydrous Na₂SO₄, filtered and concentrated. The residue was purified by flash chromatography on silica gel (Ethyl acetate in Petroleum ether=0-25%) and prep-HPLC (column: Phenomenex C18 80×40 mm×3 μm; mobile phase: [water(NH₃H₂O+NH₄HCO₃)−ACN]; gradient: 57%-87% B over 11 min) to afford 2-chloro-4-[[5-(2-chloro-4-methyl-phenoxy)-4-methyl-3-pyridyl]oxy]-3-fluoro-pyridine (400 mg, 1.05 mmol). LCMS Rₜ=1.108 min in 1.5 min chromatography, 5-95AB, ESI calcd. for C₁₈H₁₄Cl₂FN₂O₂ [M+H]⁺379.0, found 378.9.

Step 4: To a solution of 2-chloro-4-[[5-(2-chloro-4-methyl-phenoxy)-4-methyl-3-pyridyl]oxy]-3-fluoro-pyridine (400 mg, 1.05 mmol) and (sulfamoylamino) methane (174.26 mg, 1.58 mmol) in dioxane (5 mL) were added Cs₂CO₃ (1.03 g, 3.16 mmol) and [2-(2-aminophenyl)phenyl]-methylsulfonyloxy-palladium; ditert-butyl-[3,6-dimethoxy-2-(2,4,6-triisopropylphenyl)phenyl]phosphane (90.13 mg, 105.48 μmol). The mixture was stirred at 100° C. for 12 hr. The reaction was concentrated. Water (6 mL) was added and the aqueous was exctrated with DCM (2×3 mL). The combined organic layers were dried over anhydrous Na₂SO₄, filtered and concentrated. The residue was purified by flash chromatography on silica gel (Methanol in Dichloromethane=0-10%) and prep-HPLC (column: Phenomenex C18 75×30 mm×3 um; mobile phase: [water(NH₃H₂O+NH₄HCO₃)−ACN]; gradient: 20%-50% B over 8 min) to afford 4-[[5-(2-chloro-4-methyl-phenoxy)-4-methyl-3-pyridyl]oxy]-3-fluoro-N-(methylsulfamoyl)pyridin-2-amine (22.6 mg, 49.90 umol). ¹H NMR (400 MHz, CDCl₃) δ=8.13 (s, 1H), 7.99-7.83 (m, 2H), 7.80-7.36 (m, 1H), 7.31 (s, 1H), 7.09 (d, J=8.0 Hz, 1H), 6.93 (d, J=8.4 Hz, 1H), 6.37 (t, J=5.6 Hz, 1H), 5.52 (d, J=4.8 Hz, 1H), 2.79 (d, J=4.8 Hz, 3H), 2.36 (s, 3H), 2.26 (s, 3H). ¹⁹F NMR (376.5 MHz, CDCl₃) δ=−159.764. LCMS Rₜ=0.898 min in 1.5 min chromatography, 5-95AB, ESI calcd. for C₁₉H₁₉ClFN₄O₄S [M+H]⁺453.1, found 452.9.

### Example 85: N-[5-[[3-fluoro-2-(methylsulfamoylamino)-4-pyridyl]methyl]-4-methyl-3-pyridyl]-2-methyl-2-azaspiro[3.3]heptan-6-amine

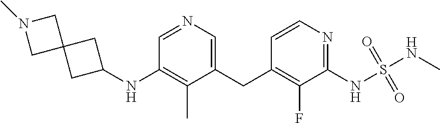

### Route

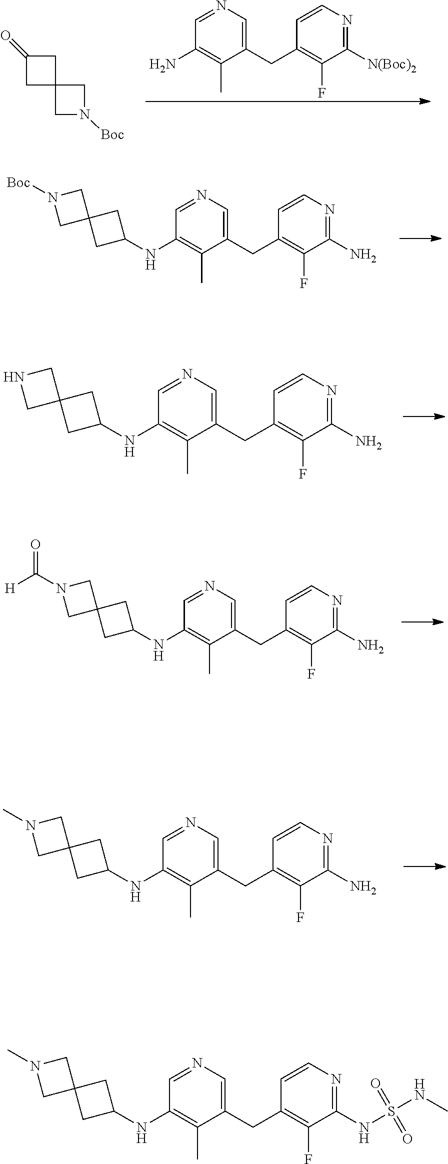

Step 1: To a solution of tert-butyl 6-oxo-2-azaspiro[3.3]heptane-2-carboxylate (976.94 mg, 4.62 mmol) in CH₂Cl₂ (10 mL) was added tert-butyl N-[4-[(5-amino-4-methyl-3-pyridyl)methyl]-3-fluoro-2-pyridyl]-N-tert-butoxycarbonyl-carbamate (500 mg, 1.16 mmol) and AcOH (347.13 mg, 5.78 mmol, 330.92 mL). After stirring at 25° C. for 20 min. NaBH(OAc)₃ (980.10 mg, 4.62 mmol) was added, the mixture was stirred at 25° C. for 12 h. The mixture was adjusted to pH=9 with NH₃·MeOH (7 M, 10 mL). The crude was purified by flash column chromatography on silica gel (MeOH in DCM=0-3%) to give tert-butyl 6-[[5-[[2-[bis(tert-butoxycarbonyl)amino]-3-fluoro-4-pyridyl]methyl]-4-methyl-3-pyridyl]amino]-2-azaspiro[3.3]heptane-2-carboxylate (550 mg, 876.15 μmol). LCMS Rt=0.815 min in 1.5 min chromatography, 5-95AB, ESI calcd. for C₃₃H₄₇FN₅O₆ [M+H]⁺628.3, found 628.3.

Step 2: To a solution of tert-butyl 6-[[5-[[2-[bis(tert-butoxycarbonyl)amino]-3-fluoro-4-pyridyl]methyl]-4-methyl-3-pyridyl]amino]-2-azaspiro[3.3]heptane-2-carboxylate (550 mg, 876.15 mmol) in HCl/MeOH (4 M, 219.04 mL). The mixture was stirred at 25° C. for 12 h. The mixture was neutralized with NH₃·MeOH (7 M, 15 mL) and purified by flash column chromatography on silica gel (MeOH in DCM=0-10%) to give N-[5-[(2-amino-3-fluoro-4-pyridyl)methyl]-4-methyl-3-pyridyl]-2-azaspiro[3.3]heptan-6-amine (200 mg, 610.88 μmol¹H NMR (400 MHz, DMSO-d₆) δ=7.84 (s, 1H), 7.71 (m, 1H), 7.60 (d, J=5.2 Hz, 1H), 6.18-6.14 (m, 3H), 4.01 (s, 2H), 3.94 (s, 2H), 3.89 (s, 2H), 3.84-3.79 (m, 1H), 3.16 (brs, 1H), 2.73-2.68 (m, 2H), 2.19-2.14 (m, 2H), 2.04 (s, 3H). ¹⁹F NMR (376.5 MHz, DMSO-d₆) δ=−144.846.

Step 3: To a solution of N-[5-[(2-amino-3-fluoro-4-pyridyl)methyl]-4-methyl-3-pyridyl]-2-azaspiro[3.3]heptan-6-amine (150 mg, 458.16 mmol) in formamide (412.71 mg, 9.16 mmol, 364.59 mL). The mixture was stirred at 140° C. for 15 min. 6-[[5-[(2-amino-3-fluoro-4-pyridyl)methyl]-4-methyl-3-pyridyl]amino]-2-azaspiro[3.3]heptane-2-carbaldehyde (162.83 mg, 458.15 mmol). LCMS Rt=0.269 min in 1.5 min chromatography, 5-95AB, ESI calcd. for C₁₉H₂₃FN₅O [M+H]⁺356.2, found 356.1.

Step 4: To a solution of 6-[[5-[(2-amino-3-fluoro-4-pyridyl)methyl]-4-methyl-3-pyridyl]amino]-2-azaspiro[3.3]heptane-2-carbaldehyde (162.83 mg, 458.15 mmol) in THF (10 mL) was added LiAlH₄ (2.5 M, 3.67 mL) at 0° C. The mixture was stirred at 25° C. for 1 h. THF (10 mL) was added and then the mixture was quenched with Na₂SO₄·10H₂O (400 mg). The mixture was filtered, and the filter cake was washed with EtOAc (5 mL×3). The filtrate was concentrated and purified by flash column chromatography on silica gel (MeOH in DCM=0-10%) to give N-[5-[(2-amino-3-fluoro-4-pyridyl)methyl]-4-methyl-3-pyridyl]-2-methyl-2-azaspiro[3.3]heptan-6-amine (60 mg, 175.73 mmol). ¹H NMR (400 MHz, CD₃Cl) δ=7.87 (s, 1H), 7.75 (s, 1H), 7.66 (d, J=5.2 Hz, 1H), 6.19 (t, J=5.2 Hz, 1H), 4.59 (brs, 2H), 3.91 (s, 2H), 3.87-3.82 (m, 1H), 3.52 (d, J=5.2 Hz, 1H), 3.37 (s, 2H), 3.27 (s, 2H), 2.74-2.69 (m, 2H), 2.34 (s, 3H), 2.04-1.98 (m, 2H), 1.94 (s, 3H). ¹⁹F NMR (376.5 MHz, CD₃Cl) δ=−145.555.

Step 5: To a solution of N-[5-[(2-amino-3-fluoro-4-pyridyl)methyl]-4-methyl-3-pyridyl]-2-methyl-2-azaspiro[3.3]heptan-6-amine (40 mg, 117.16 mmol) in MeCN (4 mL) was added Py (27.80 mg, 351.47 mmol, 28.37 mL) and N-methylsulfamoyl chloride (37.95 mg, 292.89 mmol). The mixture was stirred at 25° C. for 1 h. The mixture was concentrated and purified by Pre-HPLC (column: Welch Xtimate C18 100×40 mm×3 mm; mobile phase: [water (TFA)−ACN]; gradient: 0%-20% B over 8 min) to give N-[5-[[3-fluoro-2-(methylsulfamoylamino)-4-pyridyl]methyl]-4-methyl-3-pyridyl]-2-methyl-2-azaspiro[3.3]heptan-6-amine (7.5 mg, 17.26 mmol). ¹H NMR (400 MHz, CD₃CN) δ=11.17 (brs, 1H), 7.94-7.91 (m, 2H), 7.75 (s, 1H), 6.69-6.64 (m, 1H), 5.91 (brs, 1H), 5.19 (brs, 1H), 4.39 (d, J=10.0 Hz, 1H), 4.28 (d, J=10.0 Hz, 1H), 4.17 (s, 2H), 3.96-3.87 (m, 3H), 2.90-2.85 (m, 2H), 2.78 (s, 3H), 2.59 (s, 3H), 2.33-2.24 (m, 2H), 2.15 (s, 3H). ¹⁹F NMR (376.5 MHz, CD₃CN) δ=−141.011. LCMS Rₜ=0.760 min in 3.0 min chromatography, 5-95CD, ESI calcd. for C₂₀H₂₈FN₆O₂S [M+H]⁺435.2, found 435.1.

### Example 86: 4-[[5-[(3-chlorophenyl) methoxy]-4-methyl-3-pyridyl]methyl]-3-fluoro-N-(methylsulfamoyl)pyridin-2-amine

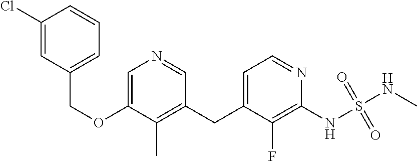 [SMILES: CNS(=O)(=O)Nc1nccc(Cc2cncc(OCc3cccc(Cl)c3)c2C)c1F]

### Route

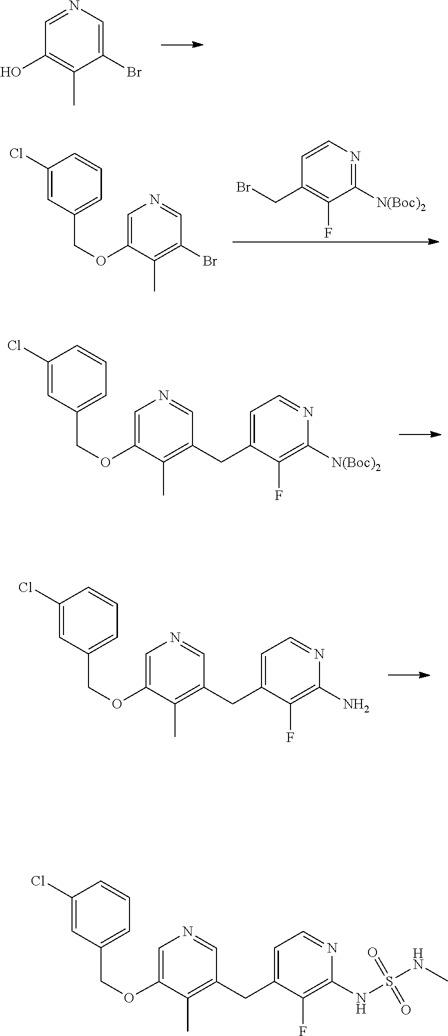

Step 1: To a solution of 5-bromo-4-methyl-pyridin-3-ol (3 g, 15.96 mmol) and Cs₂CO₃ (15.60 g, 47.87 mmol) in DMF (20 mL) was added 1-(bromomethyl)-3-chloro-benzene (2.95 g, 14.36 mmol, 1.89 mL). The mixture was stirred at 80° C. for 2 hr. Water (100 mL) was added and the mixture was extracted with EtOAc (20 mL×3). The organic layers were washed with brine (100 mL), dried over anhydrous Na₂SO₄, filtered and concentrated. The mixture was purified by flash chromatography on silica gel (Ethyl acetate in Petroleum ether=0-50%) to give 3-bromo-5-[(3-chlorophenyl) methoxy]-4-methyl-pyridine (2.1 g, 6.72 mmol). ¹H NMR (400 MHz, CDCl₃) δ=8.39 (s, 1H), 8.15 (s, 1H), 7.50-7.35 (s, 4H), 5.16 (s, 2H), 2.42 (s, 3H).

Step 2: To a solution of 3-bromo-5-[(3-chlorophenyl) methoxy]-4-methyl-pyridine (2.1 g, 6.72 mmol) and 4,4,5,5-tetramethyl-2-(4,4,5,5-tetramethyl-1,3,2-dioxaborolan-2-yl)-1,3,2-dioxaborolane (1.88 g, 7.39 mmol) in dioxane (40 mL) were added Pd(dppf)Cl₂ (344.10 mg, 470.27 μmol) and KOAc (1.98 g, 20.15 mmol), then the mixture was stirred at 110° C. for 4 hr. To the above solution were added a solution of tert-butyl N-[4-(bromomethyl)-3-fluoro-2-pyridyl]-N-tert-butoxycarbonyl-carbamate (3.76 g, 8.07 mmol) in dioxane (30 mL), Pd(dppf)Cl₂ (492.34 mg, 672.87 μmol) and K₂CO₃ (2.79 g, 20.19 mmol), then the mixture was stirred at 80° C. for 4 hr. The mixture was diluted with water (100 mL), extracted with Ethyl acetate (50 mL×2). The combined organic layers were dried over anhydrous Na₂SO₄, filtered and concentrated. The mixture was purified by flash chromatography on silica gel (Ethyl acetate in Petroleum ether=0-80%) to give tert-butyl N-tert-butoxycarbonyl-N-[4-[[5-[(3-chlorophenyl) methoxy]-4-methyl-3-pyridyl]methyl]-3-fluoro-2-pyridyl]carbamate (850 mg, 1.52 mmol). ¹H NMR (400 MHz, CDCl₃) δ=8.25-8.15 (m, 2H), 8.11 (s, 1H), 7.50-7.35 (m, 4H), 6.95-6.80 (m, 1H), 5.15 (s, 2H), 4.25-4.00 (m, 2H), 2.19 (s, 3H), 1.45-1.40 (m, 18H). ¹⁹F NMR (376.5 MHz, CDCl₃) δ=−130.94.

Step 3: A solution of tert-butyl N-tert-butoxycarbonyl-N-[4-[[5-[(3-chlorophenyl) methoxy]-4-methyl-3-pyridyl]methyl]-3-fluoro-2-pyridyl]carbamate (850 mg, 1.52 mmol) in HCl/MeOH (4 M, 9.14 mL) was stirred at 25° C. for 2 hr. The mixture was concentrated. 4-[[5-[(3-chlorophenyl) methoxy]-4-methyl-3-pyridyl]methyl]-3-fluoro-pyridin-2-amine (540 mg, 1.51 mmol). LCMS Rₜ=0.433 min in 1.5 min chromatography, 5-95AB, ESI calcd. for C₁₉H₁₈FN₃ClO [M+H]⁺358.1, found 358.2.

Step 4: To a solution of 4-[[5-[(3-chlorophenyl) methoxy]-4-methyl-3-pyridyl]methyl]-3-fluoro-pyridin-2-amine (540 mg, 1.51 mmol) and Py (1.19 g, 15.09 mmol, 1.22 mL) in ACN (5 mL) was added N-methylsulfamoyl chloride (977.70 mg, 7.55 mmol). The mixture was stirred at 25° C. for 1 hr. The solution was concentrated. The mixture was purified by prep-HPLC (column: Welch Xtimate C18 150*25 mm*5 um; mobile phase: [water(NH₃H₂O+NH₄HCO₃)−ACN]; gradient: 40%-70% B over 7 min) to give 4-[[5-[(3-chlorophenyl) methoxy]-4-methyl-3-pyridyl]methyl]-3-fluoro-N-(methylsulfamoyl)pyridin-2-amine (40 mg, 88.71 μmol). ¹H NMR (400 MHz, DMSO) δ=8.15 (s, 1H), 8.00-7.80 (m, 2H), 7.50-7.20 (m, 4H), 6.80-6.60 (m, 1H), 5.18 (s, 2H), 4.02 (s, 2H), 2.46 (s, 3H), 2.09 (s, 3H). ¹⁹F NMR (376.5 MHz, DMSO) δ=−138.74. LCMS Rₜ=0.708 min in 1.5 min chromatography, 5-95AB, ESI calcd. for C₂₀H₂₁FO₃N₄SCl [M+H]⁺451.1, found 451.0.

### Example 87: 4-[[1-(4-chloro-2-fluoro-phenyl)-3,4-dihydro-2H-quinolin-5-yl]methyl]-3-fluoro-N-(methylsulfamoyl)pyridin-2-amine

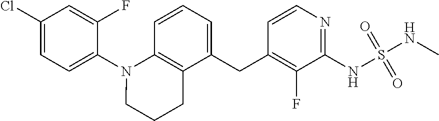 [SMILES: CNS(=O)(=O)Nc1nccc(Cc2cccc3c2CCCN3c2ccc(Cl)cc2F)c1F]

### Route

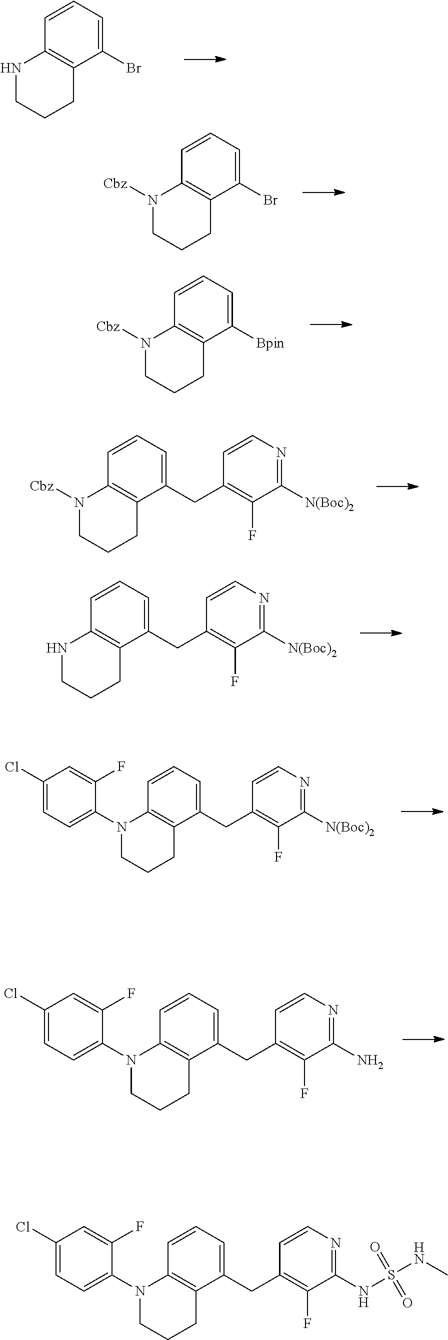

Step 1: A 100 mL three-necked round bottom flask equipped with thermometer was charged with a solution of 5-bromo-1,2,3,4-tetrahydroquinoline (2 g, 9.43 mmol) in THF (18 mL). The flask was degassed and purged with N₂ for 3 times. Then NaH (452.60 mg, 11.32 mmol, 60% purity) was added at 0° C. under N₂. After stirred at 0° C. for 30 min, CbzCl (1.77 g, 10.37 mmol, 1.48 mL) was added in one portion. Then the reaction was stirred at 25° C. for 7.5 h. Water (30 mL) was added. The mixture was extracted with EtOAc (50 mL×3). The aqueous layer were washed with H₂O (50 mL×5). The combined organic layers were washed with brine (40 mL), dried over anhydrous Na₂SO₄, filtered and concentrated. The crude product was purified by flash chromatography on silica gel (EtOAc in Petroleum ether=0-2%) to give benzyl 5-bromo-3,4-dihydro-2H-quinoline-1-carboxylate (3.26 g, 9.42 mmol). ¹H NMR (400 MHz, CDCl₃) δ=7.64 (d, J=8.0 Hz, 1H), 7.42-7.36 (m, 6H), 7.02 (t, J=8.0 Hz, 1H), 5.24 (s, 2H), 3.83-3.73 (m, 2H), 2.82 (t, J=6.8 Hz 2H), 2.03-1.91 (m, 2H).

Step 2: To a solution of benzyl 5-bromo-3,4-dihydro-2H-quinoline-1-carboxylate (1.5 g, 4.33 mmol) in dioxane (18 mL) were added 4,4,5,5-tetramethyl-2-(4,4,5,5-tetramethyl-1,3,2-dioxaborolan-2-yl)-1,3,2-dioxaborolane (1.32 g, 5.20 mmol), KOAc (1.28 g, 13.00 mmol) and Pd(dppf)Cl₂ (221.91 mg, 303.28 μmol). The mixture was stirred at 110° C. for 12 h. After cooling to 25° C., the mixture was filtered, and the filter cake was wash with EtOAc (50 mL×3). The filtrate was concentrated. The crude product was purified by flash chromatography on silica gel (EtOAc in Petroleum ether=0-3%) to give benzyl 5-(4,4,5,5-tetramethyl-1,3,2-dioxaborolan-2-yl)-3,4-dihydro-2H-quinoline-1-carboxylate (1.70 g, 4.32 mmol). ¹H NMR (400 MHz, CDCl₃) δ=7.70-7.65 (m, 1H), 7.54 (dd, J=1.2, 7.2 Hz, 1H), 7.40-7.34 (m, 5H), 7.15 (t, J=7.6 Hz, 1H), 5.22 (s, 2H), 3.79-3.73 (m, 2H), 3.06 (t, J=6.4 Hz, 2H), 1.97-1.90 (m, 2H), 1.33 (s, 12H).

Step 3: To a solution of benzyl 5-(4,4,5,5-tetramethyl-1,3,2-dioxaborolan-2-yl)-3,4-dihydro-2H-quinoline-1-carboxylate (1.7 g, 4.32 mmol) in a mixed solvent of toluene (24 mL), EtOH (6 mL) and H₂O (3 mL) were added tert-butyl N-[4-(bromomethyl)-3-fluoro-2-pyridyl]-N-tert-butoxycarbonyl-carbamate (2.10 g, 5.19 mmol), Na₂CO₃ (916.29 mg, 8.65 mmol) and Pd(PPh₃)₄ (349.65 mg, 302.58 μmol). The mixture was stirred at 80° C. for 2 h. Water (40 mL) was added. The mixture was extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-39%) to give benzyl 5-[[2-[bis(tert-butoxycarbonyl)amino]-3-fluoro-4-pyridyl]methyl]-3,4-dihydro-2H-quinoline-1-carboxylate (2 g, 3.38 mmol). ¹H NMR (400 MHz, CDCl₃) δ=8.15 (d, J=5.2 Hz, 1H), 7.69-7.58 (m, 1H), 7.43-7.33 (m, 5H), 7.13 (t, J=8.0 Hz, 1H), 6.94-6.78 (m, 2H), 5.23 (s, 2H), 4.00 (s, 2H), 3.81-3.68 (m, 2H), 2.58 (t, J=6.8 Hz, 2H), 2.00-1.85 (m, 2H), 1.41 (s, 18H). ¹⁹F NMR (376.5 MHz, CDCl₃) δ=−131.370.

Step 4: To a solution of benzyl 5-[[2-[bis(tert-butoxycarbonyl)amino]-3-fluoro-4-pyridyl]methyl]-3,4-dihydro-2H-quinoline-1-carboxylate (2 g, 3.38 mmol) in MeOH (15 mL) was added Pd/C (2.52 g, 2.37 mmol, 10% purity) under H₂. The suspension was degassed under vacuum and purged with H₂ several times. The mixture was stirred under H₂ balloon at 25° C. for 12 h. The resulting mixture was filtered, and the filter cake was wash with MeOH (30 mL×3). The filtrate was concentrated. The crude product was purified by flash chromatography on silica gel (EtOAc in Petroleum ether=0-27%) to give tert-butyl N-tert-butoxycarbonyl-N-[3-fluoro-4-(1,2,3,4-tetrahydroquinolin-5-ylmethyl)-2-pyridyl]carbamate (1 g, 2.19 mmol, 64). ¹H NMR (400 MHz, CDCl₃) δ=8.13 (d, J=5.2 Hz, 1H), 6.97-6.87 (m, 2H), 6.53-6.42 (m, 2H), 3.93 (s, 2H), 3.29-3.22 (m, 2H) 2.53 (t, J=6.4 Hz, 2H), 1.94-1.87 (m, 2H), 1.41 (s, 18H). ¹⁹F NMR (376.5 MHz, CDCl₃) δ=−131.877.

Step 5: To a solution of tert-butyl N-tert-butoxycarbonyl-N-[3-fluoro-4-(1,2,3,4-tetrahydroquinolin-5-ylmethyl)-2-pyridyl]carbamate (1.2 g, 2.62 mmol) in toluene (13 mL) were added 4-chloro-2-fluoro-1-iodo-benzene (672.58 mg, 2.62 mmol) and Cs₂CO₃ (2.56 g, 7.87 mmol) and XantPhos Pd G3 (298.47 mg, 314.73 μmol). The mixture was stirred at 110° C. for 12 h. Water (50 mL) was added. The mixture was extracted with EtOAc (40 mL×3). The combined organic layers were washed with brine (30 mL), dried over anhydrous Na₂SO₄, filtered and concentrated. The crude product was purified by flash chromatography on silica gel (EtOAc in Petroleum ether=0-26%) to give tert-butyl N-tert-butoxycarbonyl-N-[4-[[1-(4-chloro-2-fluoro-phenyl)-3,4-dihydro-2H-quinolin-5-yl]methyl]-3-fluoro-2-pyridyl]carbamate (720 mg, 1.23 mmol). ¹H NMR (400 MHz, CDCl₃) δ=8.16 (d, J=4.8 Hz, 1H), 7.24-7.14 (m, 3H), 6.96 (t, J=5.2 Hz, 1H), 6.89 (t, J=8.0 Hz, 1H), 6.51 (d, J=7.2 Hz, 1H), 6.35 (d, J=8.4 Hz, 1H), 3.99 (s, 2H), 3.53-3.43 (m, 2H), 2.65 (t, J=6.8 Hz, 2H), 2.03-1.97 (m, 2H), 1.43 (s, 18H). ¹⁹F NMR (376.5 MHz, CDCl₃) δ=−117.003, 131.533.

Step 6: To a solution of tert-butyl N-tert-butoxycarbonyl-N-[4-[[1-(4-chloro-2-fluoro-phenyl)-3,4-dihydro-2H-quinolin-5-yl]methyl]-3-fluoro-2-pyridyl]carbamate (720 mg, 1.23 mmol) in MeOH (2 mL) was added HCl/MeOH (4 M, 10 mL, 40.00 mmol). The mixture was stirred at 25° C. for 14 h. The mixture was concentrated to give 4-[[1-(4-chloro-2-fluoro-phenyl)-3,4-dihydro-2H-quinolin-5-yl]methyl]-3-fluoro-pyridin-2-amine (518.80 mg, 1.23 mmol). ¹H NMR (400 MHz, CD₃OD) δ=7.59 (d, J=6.8 Hz, 1H), 7.36-7.18 (m, 4H), 6.89 (t, J=8.0 Hz, 1H), 6.63-6.50 (m, 2H), 6.27 (d, J=8.4 Hz, 1H), 4.08 (s, 2H), 3.54-3.43 (m, 2H), 2.70 (t, J=6.4 Hz, 2H), 2.09-1.92 (m, 2H). ¹⁹F NMR (376.5 MHz, CD₃OD) δ=−118.845, −139.883

Step 7: To a solution of 4-[[1-(4-chloro-2-fluoro-phenyl)-3,4-dihydro-2H-quinolin-5-yl]methyl]-3-fluoro-pyridin-2-amine (298.80 mg, 774.42 μmol) in CH₃CN (5 mL) were added Py (306.28 mg, 3.87 mmol, 312.53 μL) and N-methylsulfamoyl chloride (150.51 mg, 1.16 mmol). The mixture was stirred at 25° C. for 0.5 h. Then N-methylsulfamoyl chloride (80.27 mg, 619.54 μmol) was added. The mixture was stirred at 25° C. for 1.5 h. The mixture was blended with another batch prepared from 200 mg of KEY INT 7. The mixture was concentrated. The crude product was purified by Prep-HPLC (column: Phenomenex C18 80×40 mm×3 μm; mobile phase: [water(NH₃H₂O+NH₄HCO₃)−ACN]; gradient: 60%-90% B over 7 min) to give 4-[[1-(4-chloro-2-fluoro-phenyl)-3,4-dihydro-2H-quinolin-5-yl]methyl]-3-fluoro-N-(methylsulfamoyl)pyridin-2-amine (362.5 mg, 756.88 μmol). ¹H NMR (400 MHz, CD₃CN) δ=7.92 (d, J=5.2 Hz, 1H), 7.34-7.16 (m, 3H), 6.87 (t, J=8.0 Hz, 1H), 6.68 (t, J=5.2 Hz, 1H), 6.53 (d, J=7.2 Hz, 1H), 6.27 (d, J=8.4 Hz, 1H), 5.89-5.70 (m, 1H), 3.98 (s, 2H), 3.54-3.41 (m, 2H), 2.69 (t, J=6.4 Hz, 2H), 2.60 (s, 3H), 2.03-1.97 (m, 2H). ¹⁹F NMR (376.5 MHz, CD₃CN) δ=−118.458, −142.161 LCMS Rₜ=0.903 min 1.5 min chromatography, 5-95AB, ESI calcd. for C₂₂H₂₂F₂N₄O₂SCl [M+H]⁺479.1, found 479.0.

### Example 88: 3-fluoro-4-[[5-(2-fluoro-4-isobutyl-anilino)-4-methyl-3-pyridyl]methyl]-N-(methylsulfamoyl)pyridin-2-amine

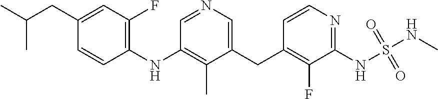 [SMILES: CNS(=O)(=O)Nc1nccc(Cc2cncc(Nc3ccc(CC(C)C)cc3F)c2C)c1F]

### Route

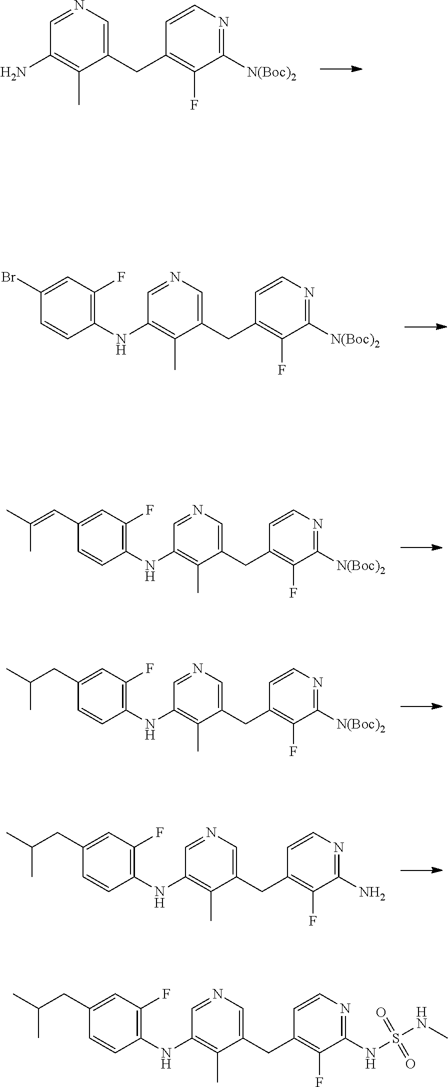

Step 1: To a solution of tert-butyl N-[4-[(5-amino-4-methyl-3-pyridyl)methyl]-3-fluoro-2-pyridyl]-N-tert-butoxycarbonyl-carbamate (800 mg, 1.85 mmol) and 4-bromo-2-fluoro-1-iodo-benzene (834.88 mg, 2.77 mmol) in dioxane (10 mL) was added Pd₂(dba)₃ (84.69 mg, 92.49 μmol), Cs₂CO₃ (843.76 mg, 2.59 mmol) and Xantphos (107.03 mg, 184.98 μmol). The mixture was stirred at 80° C. for 2 h. The mixture was diluted with water (50 mL), extracted with Ethyl acetate (20 mL×2), combined organic layers were dried over anhydrous Na₂SO₄, concentrated under reduced pressure. The mixture was purified by flash chromatography on silica gel (Ethyl acetate in Petroleum ether=0-50%) to give tert-butyl N-[4-[[5-(4-bromo-2-fluoro-anilino)-4-methyl-3-pyridyl]methyl]-3-fluoro-2-pyridyl]-N-tert-butoxycarbonyl-carbamate (600 mg, 990.97 μmol). ¹H NMR (400 MHz, CDCl₃) δ=8.32 (s, 1H), 8.21 (d, J=4.8 Hz, 1H), 8.15 (s, 1H), 7.33-7.27 (m, 1H), 7.20-7.12 (m, 1H), 6.95-6.87 (m, 1H), 6.85-6.75 (m, 1H), 5.68 (br s, 1H), 4.13 (s, 2H), 2.18 (s, 3H), 1.42 (s, 18H). ¹⁹F NMR (376.5 MHz, DMSO-d₆) δ=−130.836.

Step 2: To a solution of tert-butyl N-[4-[[5-(4-bromo-2-fluoro-anilino)-4-methyl-3-pyridyl]methyl]-3-fluoro-2-pyridyl]-N-tert-butoxycarbonyl-carbamate (500 mg, 825.81 umol) and 4,4,5,5-tetramethyl-2-(2-methylprop-1-enyl)-1,3,2-dioxaborolane (300.70 mg, 1.65 mmol) in a mixed solvent of dioxane (5 mL) and H₂O (0.25 mL) was added K₂CO₃ (228.27 mg, 1.65 mmol) and Pd(dppf)Cl₂·CH₂Cl₂ (67.44 mg, 82.58 μmol). Then the mixture was stirred at 100° C. for 3 h under N₂. The mixture was diluted with water (50 mL), extracted with Ethyl acetate (20 mL×2), combined organic layers were dried over anhydrous Na₂SO₄, concentrated under reduced pressure. The crude was purified by flash column chromatography on silica gel (MeOH in DCM=0-5%) to give tert-butyl N-tert-butoxycarbonyl-N-[3-fluoro-4-[[5-[2-fluoro-4-(2-methylprop-1-enyl) anilino]-4-methyl-3-pyridyl]methyl]-2-pyridyl]carbamate (350 mg, 602.76 mmol). ¹H NMR (400 MHz, DMSO-d₆) δ=8.24 (d, J=4.8 Hz, 1H), 8.04 (d, J=9.6 Hz, 2H), 7.54 (s, 1H), 7.26 (t, J=5.2 Hz, 1H), 7.07-7.04 (m, 1H), 6.91-6.89 (m, 1H), 6.69 (t, J=8.4 Hz, 1H), 6.16 (s, 1H), 4.18 (s, 2H), 2.07 (s, 3H), 1.85 (s, 3H), 1.82 (s, 3H), 1.33 (s, 18H). ¹⁹F NMR (376.5 MHz, DMSO-d₆) δ=−127.204, −132.426.

Step 3: To a solution of tert-butyl N-tert-butoxycarbonyl-N-[3-fluoro-4-[[5-[2-fluoro-4-(2-methylprop-1-enyl) anilino]-4-methyl-3-pyridyl]methyl]-2-pyridyl]carbamate (350 mg, 602.76 mmol) in MeOH (4 mL) was added wet Pd/C (320.73 mg, 301.38 mmol, 10% purity). The mixture was stirred at 25° C. for 12 h under 50 psi H₂. The reaction mixture was filtered and the filtrate was concentrated under reduced pressure to give tert-butyl N-tert-butoxycarbonyl-N-[3-fluoro-4-[[5-(2-fluoro-4-isobutyl-anilino)-4-methyl-3-pyridyl]methyl]-2-pyridyl]carbamate (351.22 mg, 602.77 mmol). LCMS Rt=0.871 min in 1.5 min chromatography, 5-95AB, ESI calcd. for C₃₂H₄₁F₂N₄O₄ [M+H]⁺583.3, found 583.1.

Step 4: To a solution of tert-butyl N-tert-butoxycarbonyl-N-[3-fluoro-4-[[5-(2-fluoro-4-isobutyl-anilino)-4-methyl-3-pyridyl]methyl]-2-pyridyl]carbamate (351.22 mg, 602.77 mmol) in HCl/MeOH (6 mL). The mixture was stirred at 25° C. for 12 h. The mixture was concentrated. 3-fluoro-4-[[5-(2-fluoro-4-isobutyl-anilino)-4-methyl-3-pyridyl]methyl]pyridin-2-amine (230 mg, 601.39 mmol). ¹H NMR (400 MHz, DMSO-d₆) δ=8.46 (s, 1H), 8.27 (s, 2H), 7.80 (d, J=4.8 Hz, 1H), 7.67 (s, 1H), 7.28 (t, J=7.6 Hz, 1H), 7.16 (d, J=12.0 Hz, 1H), 7.04 (d, J=7.2 Hz, 1H), 6.63 (s, 1H), 4.32 (s, 2H), 2.46 (s, 2H), 2.38 (s, 3H), 1.86 (t, J=5.6 Hz, 1H), 0.87 (d, J=6.4 Hz, 6H). ¹⁹F NMR (376.5 MHz, DMSO-d₆) δ=−121.511, −137.378.

Step 5: To a solution of 3-fluoro-4-[[5-(2-fluoro-4-isobutyl-anilino)-4-methyl-3-pyridyl]methyl]pyridin-2-amine (190 mg, 496.80 mmol) in MeCN (12 mL) were added Py (157.19 mg, 1.99 mmol, 160.39 mL) and N-methylsulfamoyl chloride (128.74 mg, 993.60 mmol). The mixture was stirred at 0° C. for 1 h. The mixture was concentrated and purified by Pre-HPLC (column: Welch Xtimate C 18 150×25 mm×5 mm; mobile phase: [water(NH₃H₂O+NH₄HCO₃)−ACN]; gradient: 50%-80% B over 7 min) to give 3-fluoro-4-[[5-(2-fluoro-4-isobutyl-anilino)-4-methyl-3-pyridyl]methyl]-N-(methylsulfamoyl)pyridin-2-amine (68.6 mg, 144.25 μmol). ¹H NMR (400 MHz, DMSO-d₆) δ=10.38 (brs, 1H), 8.00-7.96 (m, 3H), 7.34 (s, 1H), 7.02-6.99 (m, 2H), 6.83 (d, J=8.4 Hz, 1H), 6.76-6.72 (m, 2H), 4.07 (s, 2H), 2.50 (s, 3H), 2.38 (d, J=7.2 Hz, 2H), 2.06 (s, 3H), 1.83-1.77 (m, 1H), 0.85 (d, J=6.4 Hz, 6H). ¹⁹F NMR (376.5 MHz, DMSO-d₆) δ=−126.693, −138.226. LCMS Rₜ=0.772 min in 1.5 min chromatography, 5-95AB, ESI calcd. for C₂₃H₂₈F₂N₅O₂S [M+H]⁺476.2, found 476.1.

### Example 89: 3-fluoro-4-[[4-methyl-5-(5-methylthiazol-2-yl)oxy-3-pyridyl]methyl]-N-(methylsulfamoyl)pyridin-2-amine

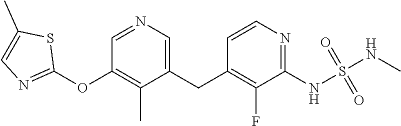 [SMILES: CNS(=O)(=O)Nc1nccc(Cc2cncc(Oc3ncc(C)s3)c2C)c1F]

### Route

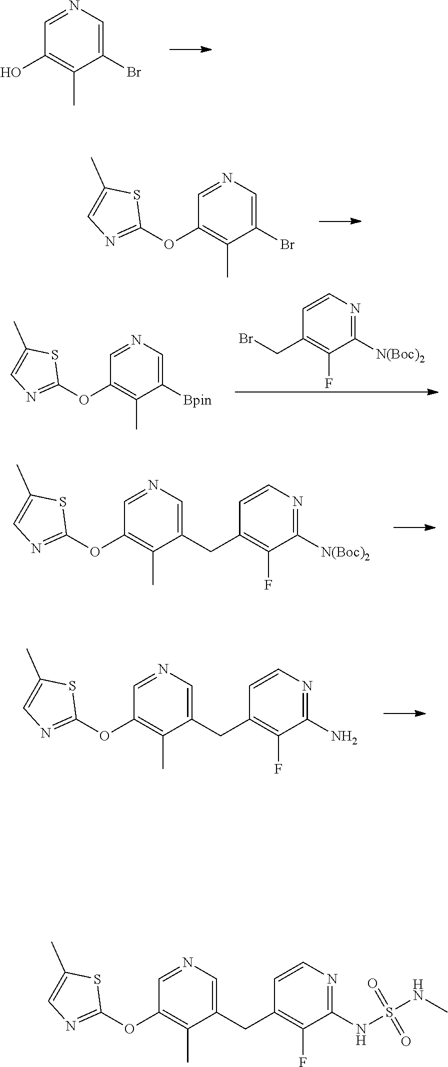

Step 1: To a solution of 5-bromo-4-methyl-pyridin-3-ol (3 g, 15.96 mmol) and 2-chloro-5-methyl-thiazole (2.34 g, 17.55 mmol) in NMP (30 mL) was added Cs₂CO₃ (15.60 g, 47.87 mmol). The mixture was stirred at 145° C. for 12 hr. Water (10 mL) was added and the aqueous was extracted with ethyl acetate (10 mL×2). The combined organic layers were dried over anhydrous Na₂SO₄, filtered and concentrated. The residue was purified by flash chromatography on silica gel (Ethyl acetate in Petroleum ether=0-25%) to afford 2-[(5-bromo-4-methyl-3-pyridyl)oxy]-5-methyl-thiazole (1.6 g, 5.61 mmol). LCMS Rₜ=3.910 min in 7.0 min chromatography, 10-80CD, ESI calcd. for C₁₀H₁₀BrN₂OS [M+H]⁺285.0, found 285.0.

Step 2: To a solution of 2-[(5-bromo-4-methyl-3-pyridyl)oxy]-5-methyl-thiazole (900 mg, 3.16 mmol) and 4,4,5,5-tetramethyl-2-(4,4,5,5-tetramethyl-1,3,2-dioxaborolan-2-yl)-1,3,2-dioxaborolane (1.60 g, 6.31 mmol) in dioxane (10 mL) were added KOAc (929.25 mg, 9.47 mmol) and Pd(dppf)Cl₂ (230.94 mg, 315.61 μmol). The mixture was stirred at 100° C. for 12 hr. The reaction was concentrated to give 5-methyl-2-[[4-methyl-5-(4,4,5,5-tetramethyl-1,3,2-dioxaborolan-2-yl)-3-pyridyl]oxy]thiazole (1.05 g, 3.16 mmol). LCMS Rₜ=0.361 min in 1.5 min chromatography, 5-95 AB, ESI calcd. for C₁₆H₂₂BN₂O₃S [M+H−C₆H₁₁]⁺251.1, found 251.0.

Step 3: To a solution of 2-[(5-bromo-4-methyl-3-pyridyl)oxy]-5-methyl-thiazole (900 mg, 3.16 mmol) and 4,4,5,5-tetramethyl-2-(4,4,5,5-tetramethyl-1,3,2-dioxaborolan-2-yl)-1,3,2-dioxaborolane (1.60 g, 6.31 mmol) in dioxane (10 mL) were added KOAc (929.25 mg, 9.47 mmol) and Pd(dppf)Cl₂ (230.94 mg, 315.61 μmol). The mixture was stirred at 100° C. for 12 hr. The reaction was concentrated to give 5-methyl-2-[[4-methyl-5-(4,4,5,5-tetramethyl-1,3,2-dioxaborolan-2-yl)-3-pyridyl]oxy]thiazole (1.05 g, 3.16 mmol, 100.00% yield). LCMS Rₜ=0.361 min in 1.5 min chromatography, 5-95 AB, ESI calcd. for C₁₆H₂₂BN₂O₃S [M+H−C₆H₁₁]⁺251.1, found 251.0.

Step 4: To a solution of tert-butyl N-tert-butoxycarbonyl-N-[3-fluoro-4-[[4-methyl-5-(5-methylthiazol-2-yl)oxy-3-pyridyl]methyl]-2-pyridyl]carbamate (843 mg, 1.59 mmol) in MeOH (4 mL) was added HCl/MeOH (4 M, 4 mL). The mixture was stirred at 25° C. for 2 hr. The reaction was concentrated. NH₃/MeOH (7 M, 5 mL) was added slowly at 0° C. adjust to pH=7. The mixture was concentrated. The residue was purified by flash chromatography on silica gel (Methanol in Dichloromethane=0-4%) to afford 3-fluoro-4-[[4-methyl-5-(5-methylthiazol-2-yl)oxy-3-pyridyl]methyl]pyridin-2-amine (200 mg, 605.36 μmol). LCMS Rₜ=0.419 min in 1.0 min chromatography, 5-95AB, ESI calcd. for C₁₆H₁₆FN₄OS [M+H]⁺331.1, found 331.1.

Step 5: To a solution of 3-fluoro-4-[[4-methyl-5-(5-methylthiazol-2-yl)oxy-3-pyridyl]methyl]pyridin-2-amine (180 mg, 544.83 μmol) in MeCN (3 mL) were added Py (129.29 mg, 1.63 mmol, 131.93 μL) and N-methylsulfamoyl chloride (84.71 mg, 653.79 umol). The mixture was stirred at 25° C. for 2 hr. The reaction was concentrated. Water (2 mL) was added and the aqueous was extracted with DCM (2 mL×2). The combined organic layers were dried over anhydrous Na₂SO₄, filtered and concentrated. The residue was purified by prep-HPLC (column: Phenomenex C18 75×30 mm×3 um; mobile phase: [water(NH₃H₂O+NH₄HCO₃)−ACN]; gradient: 15%-45% B over 8 min) to afford 3-fluoro-4-[[4-methyl-5-(5-methylthiazol-2-yl)oxy-3-pyridyl]methyl]-N-(methylsulfamoyl)pyridin-2-amine (47 mg, 110.98 μmol). ¹H NMR (400 MHz, CDCl₃) δ=8.47 (s, 1H), 8.30 (s, 1H), 7.96 (d, J=5.2 Hz, 1H), 6.82 (s, 1H), 6.60 (t, J=5.2 Hz, 1H), 5.48 (br s, 1H), 4.05 (s, 2H), 2.77 (d, J=4.0 Hz, 3H), 2.36 (s, 3H), 2.17 (s, 3H). ¹⁹F NMR (376.5 MHz, CDCl₃) δ=−142.622. LCMS Rₜ=0.647 min in 1.5 min chromatography, 5-95AB, ESI calcd. for C₁₇H₁₉FN₅O₃S₂ [M+H]⁺424.1, found 424.0.

### Example 90: 4-[[5-(4-chloro-2-fluoro-phenoxy)-4-methyl-3-pyridyl]methyl]-3-fluoro-N-(isopropylsulfamoyl)pyridin-2-amine

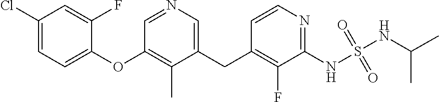 [SMILES: Cc1c(Cc2ccnc(NS(=O)(=O)NC(C)C)c2F)cncc1Oc1ccc(Cl)cc1F]

### Route

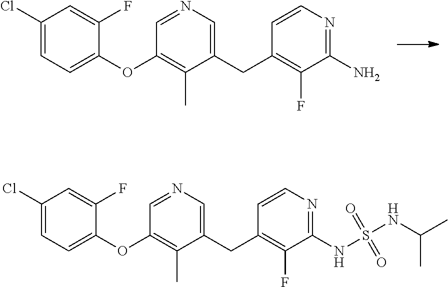 [SMILES: Cc1c(Cc2ccnc(N)c2F)cncc1Oc1ccc(Cl)cc1F ; Cc1c(Cc2ccnc(NS(=O)(=O)NC(C)C)c2F)cncc1Oc1ccc(Cl)cc1F]

To a solution of 4-[[5-(4-chloro-2-fluoro-phenoxy)-4-methyl-3-pyridyl]methyl]-3-fluoro-pyridin-2-amine (30 mg, 82.93 μmol) in CH₃CN (1 mL) were added Py (26.24 mg, 331.70 umol, 26.77 μL) and N-isopropylsulfamoyl chloride (15.68 mg, 99.51 μmol) at 0° C. The mixture was stirred at 0° C. for 1 h. Then N-isopropylsulfamoyl chloride (13.07 mg, 82.93 umol) was added at 0° C. The mixture was stirred at 0° C. for 0.5 h. The mixture was concentrated. The crude product was purified by Prep-HPLC (column: Phenomenex C18 80×40 mm×3 μm; mobile phase: [water(NH₃H₂O+NH₄HCO₃)−ACN]; gradient: 57%-87% B over 7 min) to give 4-[[5-(4-chloro-2-fluoro-phenoxy)-4-methyl-3-pyridyl]methyl]-3-fluoro-N-(isopropylsulfamoyl)pyridin-2-amine (13.8 mg, 28.58 μmol). ¹H NMR (400 MHz, CDCl₃) δ=8.25-8.15 (m, 1H), 8.06-7.95 (m, 2H), 7.57-7.30 (m, 1H), 7.25-7.21 (m, 1H), 7.17-7.08 (m, 1H), 7.00-6.88 (m, 1H), 6.59 (t, J=5.2 Hz, 1H), 5.42-5.26 (m, 1H), 4.08 (s, 2H), 3.69-3.55 (m, 1H), 2.28 (s, 3H), 1.19 (d, J=6.4 Hz, 6H). ¹⁹F NMR (376.5 MHz, CDCl₃) δ=−127.810, −142.761. LCMS Rₜ=0.864 min 1.5 min chromatography, 5-95AB, ESI calcd. for C₂₁H₂₂ClF₂N₄O₃S [M+H]⁺483.1, found 483.1.

### Example 91: 4-[[5-(2-fluoro-4-methyl-phenoxy)-4-methyl-3-pyridyl]methyl]-3-methoxy-N-(methylsulfamoyl)pyridin-2-amine

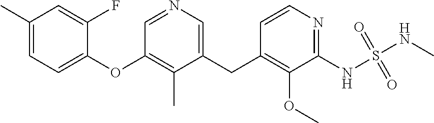

### Route

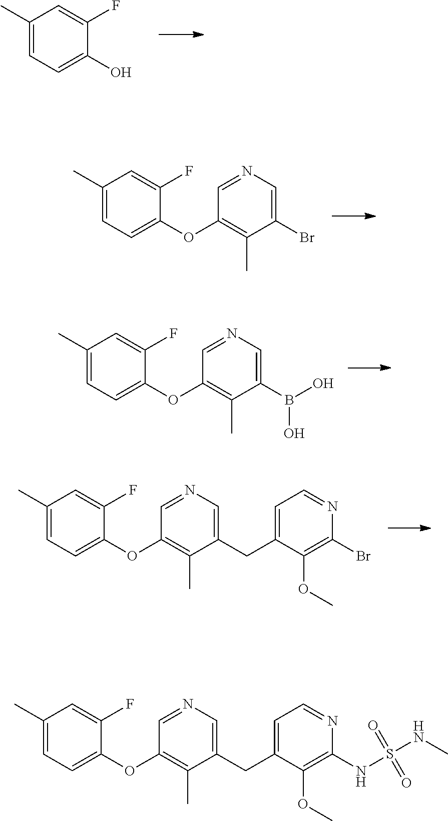

Step 1: To a solution of 3,5-dibromo-4-methyl-pyridine (10.00 g, 39.85 mmol), 2-fluoro-4-methyl-phenol (10.05 g, 79.71 mmol) in NMP (100 mL) was added Cs₂CO₃ (19.48 g, 59.78 mmol). The mixture was stirred at 130° C. for 16 h. After cooling to room temperature, the reaction mixture was filtered, partitioned between EtOAc (200 mL) and H₂O (200 mL). The organic phase was separated, washed with brine (200 mL), dried over Na₂SO₄, filtered, concentrated and purified by pre-HPLC (column: Daisogel C18 250×70 mm×10 um; mobile phase: [water(NH3H2O)−ACN]; gradient: 55%-85% B over 15 min) to give 3-bromo-5-(2-fluoro-4-methyl-phenoxy)-4-methyl-pyridine (4.00 g, 13.51 mmol). ¹H NMR (400 MHz CDCl₃) δ=8.43 (s, 1H), 7.94 (s, 1H), 7.00 (d, 1H), 6.93-6.87 (m, 2H), 2.44 (s, 3H), 2.35 (s, 3H). LCMS Rₜ=0.498 min in 0.8 min chromatography, 5-95AB, ESI calcd. for C₁₃H₁₂BrFNO [M+H]⁺296.0, found 295.9.

Step 2: A mixture of 3-bromo-5-(2-fluoro-4-methyl-phenoxy)-4-methyl-pyridine (1.5 g, 5.07 mmol), 4,4,5,5-tetramethyl-2-(4,4,5,5-tetramethyl-1,3,2-dioxaborolan-2-yl)-1,3,2-dioxaborolane (2.57 g, 10.13 mmol), Pd(dppf)Cl₂ (370.63 mg, 506.53 μmol), KOAc (1.49 g, 15.20 mmol) in dioxane (20 mL) was was stirred at 100° C. for 16 h under N₂ atmosphere. After cooling to room temperature, the reaction mixture was filtered, concentrated and purified by column chromatography (SiO₂, Petroleum ether/Ethyl acetate=3/1) to give [5-(2-fluoro-4-methyl-phenoxy)-4-methyl-3-pyridyl]boronic acid (1.20 g, 4.60 mmol). LCMS Rₜ=0.288 min in 0.8 min chromatography, 5-95AB, ESI calcd. for C₁₉H₂₄BFNO₃ [M+H]⁺262.1 found 262.0.

Step 3: A mixture of 3-bromo-5-(2-fluoro-4-methyl-phenoxy)-4-methyl-pyridine (1.5 g, 5.07 mmol), 4,4,5,5-tetramethyl-2-(4,4,5,5-tetramethyl-1,3,2-dioxaborolan-2-yl)-1,3,2-dioxaborolane (2.57 g, 10.13 mmol), Pd(dppf)Cl₂ (370.63 mg, 506.53 μmol), KOAc (1.49 g, 15.20 mmol) in dioxane (20 mL) was was stirred at 100° C. for 16 h under N₂ atmosphere. After cooling to room temperature, the reaction mixture was filtered, concentrated and purified by column chromatography (SiO₂, Petroleum ether/Ethyl acetate=3/1) to give [5-(2-fluoro-4-methyl-phenoxy)-4-methyl-3-pyridyl]boronic acid (1.20 g, 4.60 mmol). LCMS Rₜ=0.288 min in 0.8 min chromatography, 5-95AB, ESI calcd. for C₁₉H₂₄BFNO₃ [M+H]⁺262.1 found 262.0.

Step 4: To a solution of 2-bromo-4-[[5-(2-fluoro-4-methyl-phenoxy)-4-methyl-3-pyridyl]methyl]-3-methoxy-pyridine (170.00 mg, 407.41 μmol) and (sulfamoylamino) methane (89.74 mg, 814.82 μmol) in dioxane (2 mL) was added Cs₂CO₃ (398.22 mg, 1.22 mmol), tBuBrettphos-Pd-G₃ (34.81 mg, 40.74 μmol) at 25° C. The mixture was stirred at 60° C. for 5 h under N₂. After cooling to room temperature, the mixture was filtered and concentrated and purified by prep-HPLC (column: Agela DuraShell C18 150×25 mm×5 um; mobile phase: [water(NH₃H₂O)−ACN]; gradient: 14%-44% B over 10 min) 4-[[5-(2-fluoro-4-methyl-phenoxy)-4-methyl-3-pyridyl]methyl]-3-methoxy-N-(methylsulfamoyl)pyridin-2-amine (7.00 mg, 15.68 μmol). ¹H NMR (400 MHz DMSO-d₆) δ=8.12 (s, 1H), 7.92-7.82 (m, 2H), 7.20 (d, J=12.4 Hz, 1H), 7.03-6.98 (m, 1H), 6.97-6.92 (m, 1H), 6.53 (br s, 1H), 4.05 (s, 2H), 3.69 (s, 3H), 2.45 (s, 3H), 2.29 (s, 3H), 2.14 (s, 3H). ¹⁹F NMR (376.5 MHz CDCl₃) δ=−113.057 ppm. LCMS Rₜ=1.437 min in 3.0 min chromatography, 5-95AB, ESI calcd. for C₂₁H₂₄FN₄O₄S [M+H]⁺446.5, found 447.1.

### Example 92: 3-fluoro-4-[[4-methyl-5-[(2-methyl-2-azaspiro[3.3]heptan-6-yl)oxy]-3-pyridyl]methyl]-N-(methylsulfamoyl)pyridin-2-amine

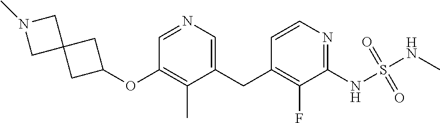

### Route

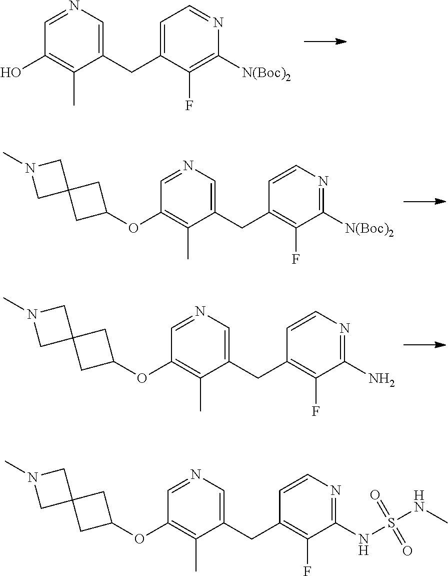

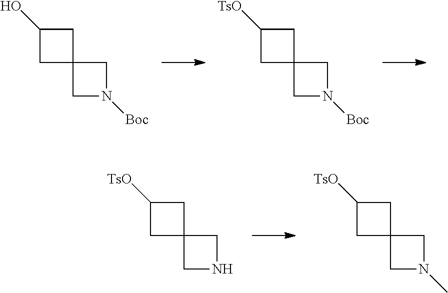 [SMILES: CC(C)(C)OC(=O)N1CC2(CC(O)C2)C1 ; Cc1ccc(S(=O)(=O)OC2CC3(C2)CN(C(=O)OC(C)(C)C)C3)cc1 ; Cc1ccc(S(=O)(=O)OC2CC3(CNC3)C2)cc1 ; Cc1ccc(S(=O)(=O)OC2CC3(C2)CN(C)C3)cc1]

Step 1: A solution of tert-butyl 6-hydroxy-2-azaspiro[3.3]heptane-2-carboxylate (4 g, 18.76 mmol) and TsCl (3.97 g, 56.27 mmol) in DCM (35 mL) was degassed and purged with N₂ for 3 times, then TEA (9.49 g, 93.78 mmol, 13.05 mL) and DMAP (687.40 mg, 5.63 mmol) were added in portions under N₂ at 0° C. The mixture was stirred at 0° C. for 1 h then warmed to 25° C. stirred for 15 h under N₂. The mixture was poured into water (50 mL), extracted with DCM (30 mL×2). The combined organic layers were washed with brine (50 mL), dried over anhydrous Na₂SO₄, filtered and concentrated. The residue was purified by flash chromatography on silica gel (Ethyl acetate in Petroleum ether=0-25%) to give tert-butyl 6-(p-tolylsulfonyloxy)-2-azaspiro[3.3]heptane-2-carboxylate (6.1 g, 16.60 mmol). ¹H NMR (400 MHz, CDCl₃) δ=7.76 (d, J=8.0 Hz, 2H), 7.34 (d, J=8.0 Hz, 2H), 4.75-4.60 (m, 1H), 3.84 (s, 4H), 2.53-2.42 (m, 5H), 2.35-2.24 (m, 2H), 1.40 (s, 9H)

Step 2: A solution of tert-butyl 6-hydroxy-2-azaspiro[3.3]heptane-2-carboxylate (4 g, 18.76 mmol) and TsCl (3.97 g, 56.27 mmol) in DCM (35 mL) was degassed and purged with N₂ for 3 times, then TEA (9.49 g, 93.78 mmol, 13.05 mL) and DMAP (687.40 mg, 5.63 mmol) were added in portions under N₂ at 0° C. The mixture was stirred at 0° C. for 1 h then warmed to 25° C. stirred for 15 h under N₂. The mixture was poured into water (50 mL), extracted with DCM (30 mL×2). The combined organic layers were washed with brine (50 mL), dried over anhydrous Na₂SO₄, filtered and concentrated. The residue was purified by flash chromatography on silica gel (Ethyl acetate in Petroleum ether=0-25%) to give tert-butyl 6-(p-tolylsulfonyloxy)-2-azaspiro[3.3]heptane-2-carboxylate (6.1 g, 16.60 mmol). ¹H NMR (400 MHz, CDCl₃) δ=7.76 (d, J=8.0 Hz, 2H), 7.34 (d, J=8.0 Hz, 2H), 4.75-4.60 (m, 1H), 3.84 (s, 4H), 2.53-2.42 (m, 5H), 2.35-2.24 (m, 2H), 1.40 (s, 9H)

Step 3: To a solution of 2-azaspiro[3.3]heptan-6-yl 4-methylbenzenesulfonate (2 g, 7.48 mmol) and formaldehyde (1.21 g, 14.96 mmol, 1.11 mL, 37% purity) in DCM (20 mL) was added NaBH(OAc)₃ (4.76 g, 22.44 mmol) in portions under N₂. The mixture was stirred at 25° C. for 1 h. The mixture was filtered and the filtrate was concentrated. The residue was purified by flash chromatography on silica gel (Methanol in Dichloromethane=0-15%) to give (2-methyl-2-azaspiro[3.3]heptan-6-yl) 4-methylbenzenesulfonate (1.8 g, 6.40 mmol). ¹H NMR (400 MHz, CDCl₃) δ=7.73 (d, J=8.0 Hz, 2H), 7.33 (d, J=8.0 Hz, 2H), 4.69-4.57 (m, 1H), 3.61 (d, J=6.8 Hz, 4H), 2.58-2.47 (m, 5H), 2.44 (s, 3H), 2.36-2.28 (m, 2H), 1.97 (s, 3H).

Step 4: To a solution of 2-azaspiro[3.3]heptan-6-yl 4-methylbenzenesulfonate (2 g, 7.48 mmol) and formaldehyde (1.21 g, 14.96 mmol, 1.11 mL, 37% purity) in DCM (20 mL) was added NaBH(OAc)₃ (4.76 g, 22.44 mmol) in portions under N₂. The mixture was stirred at 25° C. for 1 h. The mixture was filtered and the filtrate was concentrated. The residue was purified by flash chromatography on silica gel (Methanol in Dichloromethane=0-15%) to give (2-methyl-2-azaspiro[3.3]heptan-6-yl) 4-methylbenzenesulfonate (1.8 g, 6.40 mmol). ¹H NMR (400 MHz, CDCl₃) δ=7.73 (d, J=8.0 Hz, 2H), 7.33 (d, J=8.0 Hz, 2H), 4.69-4.57 (m, 1H), 3.61 (d, J=6.8 Hz, 4H), 2.58-2.47 (m, 5H), 2.44 (s, 3H), 2.36-2.28 (m, 2H), 1.97 (s, 3H).

Step 5: A solution of tert-butyl N-tert-butoxycarbonyl-N-[3-fluoro-4-[[4-methyl-5-[(2-methyl-2-azaspiro[3.3]heptan-6-yl)oxy]-3-pyridyl]methyl]-2-pyridyl]carbamate (400 mg, 737.14 μmol) in HCl/MeOH (4 M, 3 mL) was stirred at 25° C. for 1 h. The mixture was concentrated. The mixture was diluted with MeOH (5 mL), then NH₃/MeOH (7 M, 4 mL) was added. The mixture was concentrated. The residue was purified by flash chromatography on silica gel (DCM/MeOH (400 mL, 10:1 with 4 mL NH₃·H₂O) in DCM=0-70%) to give 3-fluoro-4-[[4-methyl-5-[(2-methyl-2-azaspiro[3.3]heptan-6-yl)oxy]-3-pyridyl]methyl]pyridin-2-amine (230 mg, 671.71 μmol). ¹H NMR (400 MHz, DMSO-d₆) δ=7.98 (d, J=13.2 Hz, 2H), 7.60 (d, J=5.2 Hz, 1H), 6.20-6.06 (m, 3H), 4.76-4.63 (m, 1H), 3.91 (s, 2H), 3.15 (s, 2H), 3.07 (s, 2H), 2.71-2.58 (m, 2H), 2.15 (s, 3H), 2.13-2.08 (m, 2H), 2.04 (s, 3H). ¹⁹F NMR (376.5 MHz, DMSO-d₆) δ=−145.094

Step 6: A solution of tert-butyl N-tert-butoxycarbonyl-N-[3-fluoro-4-[[4-methyl-5-[(2-methyl-2-azaspiro[3.3]heptan-6-yl)oxy]-3-pyridyl]methyl]-2-pyridyl]carbamate (400 mg, 737.14 μmol) in HCl/MeOH (4 M, 3 mL) was stirred at 25° C. for 1 h. The mixture was concentrated. The mixture was diluted with MeOH (5 mL), then NH₃/MeOH (7 M, 4 mL) was added. The mixture was concentrated. The residue was purified by flash chromatography on silica gel (DCM/MeOH (400 mL, 10:1 with 4 mL NH₃·H₂O) in DCM=0-70%) to give 3-fluoro-4-[[4-methyl-5-[(2-methyl-2-azaspiro[3.3]heptan-6-yl)oxy]-3-pyridyl]methyl]pyridin-2-amine (230 mg, 671.71 μmol). ¹H NMR (400 MHz, DMSO-d₆) δ=7.98 (d, J=13.2 Hz, 2H), 7.60 (d, J=5.2 Hz, 1H), 6.20-6.06 (m, 3H), 4.76-4.63 (m, 1H), 3.91 (s, 2H), 3.15 (s, 2H), 3.07 (s, 2H), 2.71-2.58 (m, 2H), 2.15 (s, 3H), 2.13-2.08 (m, 2H), 2.04 (s, 3H). ¹⁹F NMR (376.5 MHz, DMSO-d₆) δ=−145.094

### Example 93: 4-[[5-(4-chloro-2-fluoro-phenoxy)-4-methyl-3-pyridyl]methyl]-3-methoxy-N-(methylsulfamoyl)pyridin-2-amine

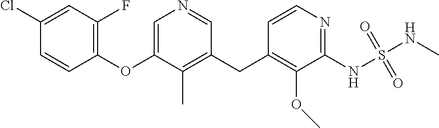 [SMILES: CNS(=O)(=O)Nc1nccc(Cc2cncc(Oc3ccc(Cl)cc3F)c2C)c1OC]

### Route

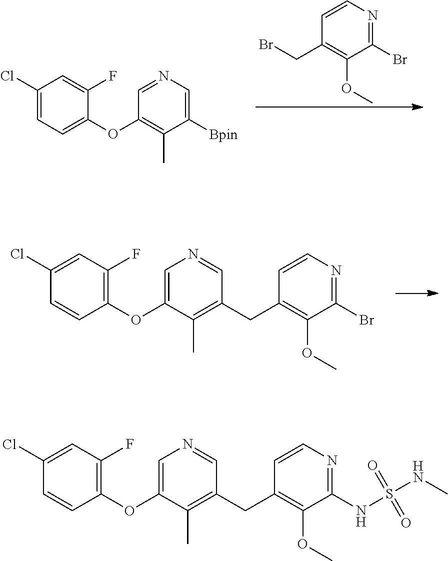

Step 1: To a solution of 3-(4-chloro-2-fluoro-phenoxy)-4-methyl-5-(4,4,5,5-tetramethyl-1,3,2-dioxaborolan-2-yl)pyridine (2.3 g, 5.44 mmol, example 81) in toluene (26 mL), EtOH (6 mL) and H₂O (2 mL) were added 2-bromo-4-(bromomethyl)-3-methoxy-pyridine (1.38 g, 4.90 mmol), Na₂CO₃ (1.15 g, 10.88 mmol) and Pd(PPh₃)₄ (628.60 mg, 543.98 μmol). The mixture was stirred at 100° C. for 1 h. Water (30 mL) was added. The mixture was extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-46%) to give 2-bromo-4-[[5-(4-chloro-2-fluoro-phenoxy)-4-methyl-3-pyridyl]methyl]-3-methoxy-pyridine (790 mg, 1.80 mmol). ¹H NMR (400 MHz, CDCl₃) δ=8.19 (s, 1H), 8.08-7.99 (m, 2H), 7.23 (dd, J=2.4, 10.4 Hz, 1H), 7.14-7.05 (m, 1H), 6.89 (t, J=8.8 Hz, 1H), 6.79 (d, J=5.2 Hz, 1H), 4.10 (s, 2H), 3.90 (s, 3H), 2.17 (s, 3H). ¹⁹F NMR (376.5 MHz, CDCl₃) δ=−128.186.

Step 2: To a solution of 2-bromo-4-[[5-(4-chloro-2-fluoro-phenoxy)-4-methyl-3-pyridyl]methyl]-3-methoxy-pyridine (300 mg, 685.42 μmol) in dioxane (3 mL) were added (sulfamoylamino) methane (113.23 mg, 1.03 mmol), Cs₂CO₃ (669.97 mg, 2.06 mmol) and TBUBRETTPHOS PD G3 (292.82 mg, 342.71 μmol). The mixture was stirred at 100° C. for 1 h. The mixture was concentrated. The crude product was purified by flash chromatography on silica gel (MeOH in Dichloromethane=0-8%) and Prep-HPLC (column: Phenomenex C18 80×40 mm×3 um; mobile phase: [water(NH₃H₂O+NH₄HCO₃)−ACN]; gradient: 52%-82% B over 7 min) to give 4-[[5-(4-chloro-2-fluoro-phenoxy)-4-methyl-3-pyridyl]methyl]-3-methoxy-N-(methylsulfamoyl)pyridin-2-amine (20.6 mg, 44.12 μmol, 6.44% yield). ¹H NMR (400 MHz, CD₃CN) δ=8.20 (s, 1H), 8.06 (s, 1H), 7.96-7.80 (br s, 1H), 7.37 (dd, J=2.8, 10.8 Hz, 1H), 7.20-7.09 (m, 1H), 6.91 (t, J=9.2 Hz, 1H), 6.65-6.43 (m, 1H), 5.98-5.73 (br s, 1H), 4.10 (s, 2H), 3.79 (s, 3H), 2.57 (s, 3H), 2.13 (s, 3H). ¹⁹F NMR (376.5 MHz, CD₃CN) δ=−131.044 LCMS Rₜ=0.748 min in 1.5 min chromatography, 5-95AB, ESI calcd. for C₂₀H₂₁ClFN₄O₄S [M+H]⁺466.9, found 466.9.

### Example 94: 3-fluoro-4-[[5-(2-fluoro-4-methoxy-phenoxy)-4-methyl-3-pyridyl]methyl]-N-(methylsulfamoyl)pyridin-2-amine

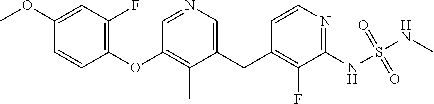

### Route

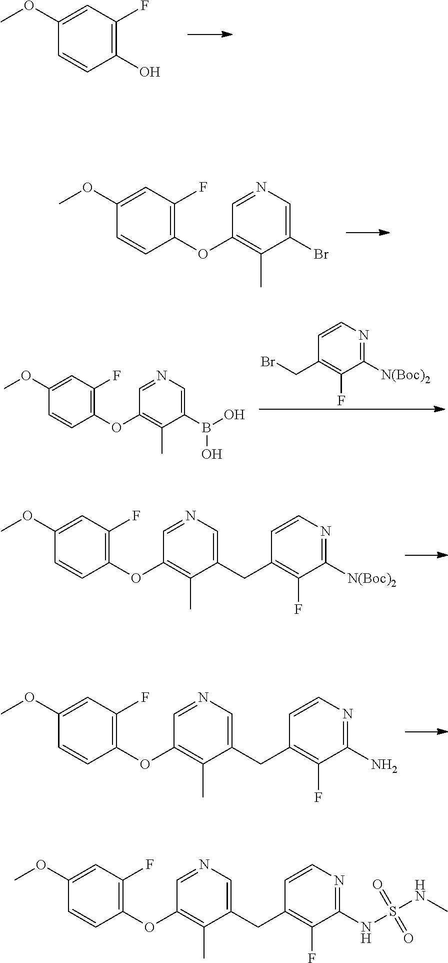

Step 1: To a solution of 2-fluoro-4-methoxy-phenol (4.25 g, 29.89 mmol) and 3,5-dibromo-4-methyl-pyridine (5.00 g, 19.93 mmol) in NMP (10 mL) was added Cs₂CO₃ (9.74 g, 29.89 mmol) at 25° C. The mixture was stirred at 140° C. for 16 h under N₂. After cooling to room temperature, the reaction mixture was poured into water (20 mL), extracted with EtOAc (20 mL×3). Then the combined organic phase was washed with H₂O (20 mL×3) and brine (10 mL×1), dried over anhydrous Na₂SO₄, filtered and concentrated. The residue was purified by flash chromatography on silica gel (EtOAc in Petroleum ether=0-20%) to give 3-bromo-5-(2-fluoro-4-methoxy-phenoxy)-4-methyl-pyridine (5 g, 16.02 mmol). LCMS Rₜ=0.479 min in 0.8 min chromatography, 5-95AB, ESI calcd. for C₁₃H₁₂BrFNO₂ [M+H]⁺314.0, found 313.8 ¹H NMR (400 MHz CDCl₃-d) δ=8.40 (s, 1H), 7.87 (s, 1H), 7.04-6.96 (m, 1H), 6.78-6.74 (m, 1H), 6.67-6.63 (m, 1H), 3.73 (s, 2H), 2.47 (s, 3H).

Step 2: To a solution of 3-bromo-5-(2-fluoro-4-methoxy-phenoxy)-4-methyl-pyridine (5.00 g, 16.02 mmol) in dioxane (50 mL) was added Pd(dppf)Cl₂ (1.17 g, 1.60 mmol), KOAc (4.72 g, 48.06 mmol), B₂Pin₂ (8.14 g, 32.04 mmol) at 25° C. The mixture was stirred at 100° C. for 12 h under N₂. After cooling to room temperature, the mixture was poured into water (50 mL), extracted with EtOAc (50 mL×3). Then the combined organic phase was washed with H₂O (50 mL×3) and brine (50 mL), dried over anhydrous Na₂SO₄, filtered and concentrated under reduced pressure. The residue was purified by flash chromatography on silica gel (EtOAc in Petroleum ether=0-70%) to give [5-(2-fluoro-4-methoxy-phenoxy)-4-methyl-3-pyridyl]boronic acid (3.50 g, 12.63 mmol). LCMS Rₜ=0.278 min in 0.8 min chromatography, 5-95AB, ESI calcd. for C₁₃H₁₄BFNO₄ [M+H]⁺278.0, found 278.1. ¹H NMR (400 MHz CDCl₃-d) δ=8.56 (s, 1H), 8.02-7.92 (m, 1H), 6.86 (t, J=8.8 Hz, 1H), 6.71 (dd, J=2.8, 12.0 Hz, 1H), 6.62-6.54 (m, 1H), 3.75-3.73 (m, 1H), 2.50 (s, 2H).

Step 3: To a solution of [5-(2-fluoro-4-methoxy-phenoxy)-4-methyl-3-pyridyl]boronic acid (2.00 g, 7.22 mmol) tert-butyl N-[4-(bromomethyl)-3-fluoro-2-pyridyl]-N-tert-butoxycarbonyl-carbamate (2.34 g, 5.78 mmol) in H₂O (1 mL), EtOH (6 mL), toluene (6 mL) was added Cs₂CO₃ (7.06 g, 21.66 mmol) cyclopentyl(diphenyl) phosphane; dichloromethane; dichloropalladium; iron (589.51 mg, 721.88 μmol) at 25° C. The mixture was stirred at 80° C. for 2 h. After cooling to room temperature, the mixture was poured into water (5 ml), extracted with EtOAc (5 mL×3). The combined organic phase was washed with H₂O (5 mL×3) and brine (5 mL), dried over anhydrous Na₂SO₄, filtered and concentrated under reduced pressure. This batch was combined with another batch prepared from [5-(2-fluoro-4-methoxy-phenoxy)-4-methyl-3-pyridyl]boronic acid (1.00 g, 3.61 mmol). The residue was purified by flash chromatography on silica gel (EtOAc in Petroleum ether=0-50%) to give tert-butyl N-tert-butoxycarbonyl-N-[3-fluoro-4-[[5-(2-fluoro-4-methoxy-phenoxy)-4-methyl-3-pyridyl]methyl]-2-pyridyl]carbamate (1.40 g, 2.51 mmol). LCMS Rₜ=0.476 min in 0.8 min chromatography, 5-95AB, ESI calcd. for C₂₉H₃₄F₂N₃O₆ [M+H]⁺558.2, found 558.1. ¹H NMR (400 MHz CDCl3-d) δ=8.19 (d, J=4.8 Hz, 1H), 8.13 (s, 1H), 7.94 (s, 1H), 7.01-6.96 (m, 1H), 6.91 (t, J=10.0 Hz, 1H), 6.79-6.72 (m, 1H), 6.69-6.63 (m, 1H), 4.14-4.10 (m, 2H), 3.19 (s, 3H), 2.23 (s, 3H), 1.41 (s, 18H).

Step 4: To a solution of [5-(2-fluoro-4-methoxy-phenoxy)-4-methyl-3-pyridyl]boronic acid (2.00 g, 7.22 mmol) tert-butyl N-[4-(bromomethyl)-3-fluoro-2-pyridyl]-N-tert-butoxycarbonyl-carbamate (2.34 g, 5.78 mmol) in H₂O (1 mL), EtOH (6 mL), toluene (6 mL) was added Cs₂CO₃ (7.06 g, 21.66 mmol) cyclopentyl(diphenyl) phosphane; dichloromethane; dichloropalladium; iron (589.51 mg, 721.88 μmol) at 25° C. The mixture was stirred at 80° C. for 2 h. After cooling to room temperature, the mixture was poured into water (5 ml), extracted with EtOAc (5 mL×3). The combined organic phase was washed with H₂O (5 mL×3) and brine (5 mL), dried over anhydrous Na₂SO₄, filtered and concentrated under reduced pressure. This batch was combined with another batch prepared from [5-(2-fluoro-4-methoxy-phenoxy)-4-methyl-3-pyridyl]boronic acid (1.00 g, 3.61 mmol). The residue was purified by flash chromatography on silica gel (EtOAc in Petroleum ether=0-50%) to give tert-butyl N-tert-butoxycarbonyl-N-[3-fluoro-4-[[5-(2-fluoro-4-methoxy-phenoxy)-4-methyl-3-pyridyl]methyl]-2-pyridyl]carbamate (1.40 g, 2.51 mmol). LCMS Rₜ=0.476 min in 0.8 min chromatography, 5-95AB, ESI calcd. for C₂₉H₃₄F₂N₃O₆ [M+H]⁺558.2, found 558.1. ¹H NMR (400 MHz CDCl3-d) δ=8.19 (d, J=4.8 Hz, 1H), 8.13 (s, 1H), 7.94 (s, 1H), 7.01-6.96 (m, 1H), 6.91 (t, J=10.0 Hz, 1H), 6.79-6.72 (m, 1H), 6.69-6.63 (m, 1H), 4.14-4.10 (m, 2H), 3.19 (s, 3H), 2.23 (s, 3H), 1.41 (s, 18H).

Step 5: To a solution of [5-(2-fluoro-4-methoxy-phenoxy)-4-methyl-3-pyridyl]boronic acid (2.00 g, 7.22 mmol) tert-butyl N-[4-(bromomethyl)-3-fluoro-2-pyridyl]-N-tert-butoxycarbonyl-carbamate (2.34 g, 5.78 mmol) in H₂O (1 mL), EtOH (6 mL), toluene (6 mL) was added Cs₂CO₃ (7.06 g, 21.66 mmol) cyclopentyl(diphenyl) phosphane; dichloromethane; dichloropalladium; iron (589.51 mg, 721.88 μmol) at 25° C. The mixture was stirred at 80° C. for 2 h. After cooling to room temperature, the mixture was poured into water (5 ml), extracted with EtOAc (5 mL×3). The combined organic phase was washed with H₂O (5 mL×3) and brine (5 mL), dried over anhydrous Na₂SO₄, filtered and concentrated under reduced pressure. This batch was combined with another batch prepared from [5-(2-fluoro-4-methoxy-phenoxy)-4-methyl-3-pyridyl]boronic acid (1.00 g, 3.61 mmol). The residue was purified by flash chromatography on silica gel (EtOAc in Petroleum ether=0-50%) to give tert-butyl N-tert-butoxycarbonyl-N-[3-fluoro-4-[[5-(2-fluoro-4-methoxy-phenoxy)-4-methyl-3-pyridyl]methyl]-2-pyridyl]carbamate (1.40 g, 2.51 mmol). LCMS Rₜ=0.476 min in 0.8 min chromatography, 5-95AB, ESI calcd. for C₂₉H₃₄F₂N₃O₆ [M+H]⁺558.2, found 558.1. ¹H NMR (400 MHz CDCl3-d) δ=8.19 (d, J=4.8 Hz, 1H), 8.13 (s, 1H), 7.94 (s, 1H), 7.01-6.96 (m, 1H), 6.91 (t, J=10.0 Hz, 1H), 6.79-6.72 (m, 1H), 6.69-6.63 (m, 1H), 4.14-4.10 (m, 2H), 3.19 (s, 3H), 2.23 (s, 3H), 1.41 (s, 18H).

### Example 95: N-(ethylsulfamoyl)-3-fluoro-4-[[5-(2-fluoro-4-methyl-anilino)-4-methyl-3-pyridyl]methyl]pyridin-2-amine

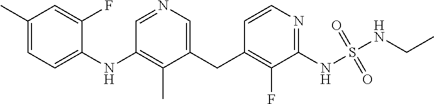 [SMILES: CCNS(=O)(=O)Nc1nccc(Cc2cncc(Nc3ccc(C)cc3F)c2C)c1F]

### Route

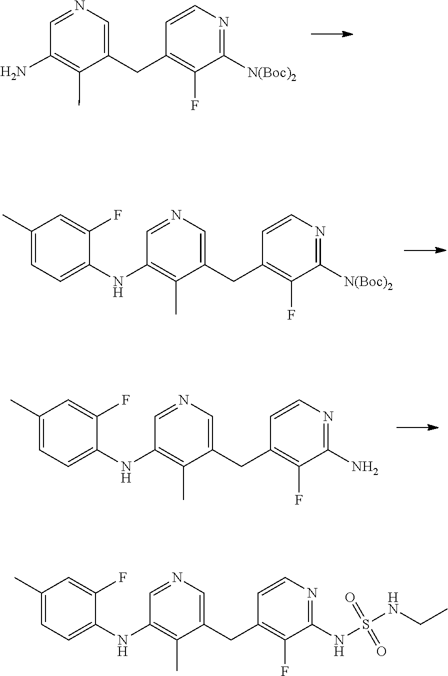

Step 1: To a solution of tert-butyl N-[4-[(5-amino-4-methyl-3-pyridyl)methyl]-3-fluoro-2-pyridyl]-N-tert-butoxycarbonyl-carbamate (500 mg, 1.16 mmol) in dioxane (10 mL) were added 2-fluoro-1-iodo-4-methyl-benzene (300.16 mg, 1.27 mmol), Pd(OAc)₂ (25.96 mg, 115.61 mmol), Xantphos (66.89 mg, 115.61 mmol) and Cs₂CO₃ (1.13 g, 3.47 mmol). The mixture was stirred at 100° C. for 12 h. Water (10 mL) was added and the mixture were extracted with EtOAc (10 ml×2). The organic layer was dried over anhydrous Na₂SO₄, filtered and concentrated. tert-butyl N-tert-butoxycarbonyl-N-[3-fluoro-4-[[5-(2-fluoro-4-methyl-anilino)-4-methyl-3-pyridyl]methyl]-2-pyridyl]carbamate (624.99 mg, 16 mmol). LCMS Rt=1.84 min in 3.0 min chromatography, 5-95CD, ESI calcd. for C₂₉H₃₅F₂N₄O₄ [M+H]⁺541.3, found 541.2.

Step 2: A mixture of tert-butyl N-tert-butoxycarbonyl-N-[3-fluoro-4-[[5-(2-fluoro-4-methyl-anilino)-4-methyl-3-pyridyl]methyl]-2-pyridyl]carbamate (624.99 mg, 1.16 mmol) in HCl/MeOH (4 M, 10 mL). The mixture was stirred at 25° C. for 12 h. The mixture was neutralized with NH₃·MeOH (7 M, 6 mL) and purified by flash column chromatography on silica gel (EtOAc in petroleum ether=0-80%) to give 3-fluoro-4-[[5-(2-fluoro-4-methyl-anilino)-4-methyl-3-pyridyl]methyl]pyridin-2-amine (130 mg, 381.94 mmol). ¹H NMR (400 MHz, CDCl₃) δ=8.36 (s, 1H), 8.12 (s, 1H), 7.73 (d, J=5.2 Hz, 1H), 6.93 (d, J=12.4 Hz, 1H), 6.82 (d, J=4.8 Hz, 2H), 6.26 (t, J=5.2 Hz, 1H), 4.60 (s, 2H), 3.97 (s, 2H), 2.30 (s, 3H), 2.13 (s, 3H). ¹⁹F NMR (376.5 MHz, CDCl₃) δ=−132.290, −145.427.

Step 3: To a solution of 3-fluoro-4-[[5-(2-fluoro-4-methyl-anilino)-4-methyl-3-pyridyl]methyl]pyridin-2-amine (40 mg, 117.52 mmol) in MeCN (2 mL) was added Py (37.18 mg, 470.08 mmol, 37.94 mL) and N-ethylsulfamoyl chloride (33.75 mg, 235.04 mmol). The mixture was stirred at 25° C. for 0.5 h. The mixture was concentrated and purified by Pre-HPLC (column: Welch Xtimate C18 150×25 mm×5 mm; mobile phase: [water(NH₃H₂O+NH₄HCO₃)−ACN]; gradient: 30%-60% B over 7 min) and SFC (column: DAICEL CHIRALPAK AD (250 mm×30 mm, 10 mm); mobile phase: [CO₂-EtOH (0.1% NH₃H₂O)]; B %:40% %, isocratic elution mode) to give N-(ethylsulfamoyl)-3-fluoro-4-[[5-(2-fluoro-4-methyl-anilino)-4-methyl-3-pyridyl]methyl]pyridin-2-amine (6.6 mg, 14.75 mmol). ¹H NMR (400 MHz, CDCl₃) δ=8.37 (s, 1H), 8.10 (s, 1H), 7.96 (d, J=5.2 Hz, 1H), 7.39-7.31 (m, 1H), 6.96-6.84 (m, 3H), 6.58 (t, J=5.2 Hz, 1H), 5.45 (brs, 1H), 5.30 (brs, 1H), 4.04 (s, 2H), 3.16-3.13 (m, 2H), 2.31 (s, 3H), 2.13 (s, 3H), 1.25-1.14 (m, 3H). ¹⁹F NMR (376.5 MHz, CDCl₃) δ=−131.887, −143.028. LCMS Rₜ=0.466 min in 1.0 min chromatography, 5-95AB, ESI calcd. for C₂₁H₂₄F₂N₅O₂S [M+H]⁺448.2, found 448.2.

### Example 96: 3-fluoro-4-[[5-(2-fluoro-4-methyl-anilino)-4-methyl-3-pyridyl]methyl]-2-(sulfamoylamino)pyridine

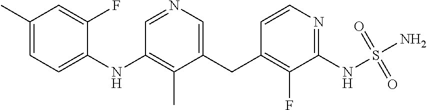 [SMILES: Cc1ccc(Nc2cncc(Cc3ccnc(NS(N)(=O)=O)c3F)c2C)c(F)c1]

### Route

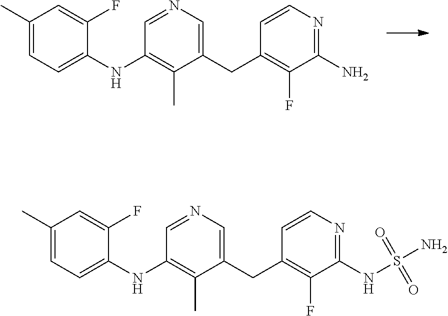 [SMILES: Cc1ccc(Nc2cncc(Cc3ccnc(N)c3F)c2C)c(F)c1 ; Cc1ccc(Nc2cncc(Cc3ccnc(NS(N)(=O)=O)c3F)c2C)c(F)c1]

Step 1: To a solution of 3-fluoro-4-[[5-(2-fluoro-4-methyl-anilino)-4-methyl-3-pyridyl]methyl]pyridin-2-amine (60 mg, 176.28 mmol, example 95) in MeCN (5 mL) was added Py (55.77 mg, 705.12 mmol, 56.91 mL) and sulfamoyl chloride (122.20 mg, 1.06 mmol). The mixture was stirred at 25° C. for 0.5 h. The mixture was concentrated and purified by Prep-HPLC (column: Welch Xtimate C18 150×25 mm×5 mm; mobile phase: [water(NH₃H₂O+NH₄HCO₃)−ACN]; gradient: 20%-50% B over 7 min) and SFC (column: DAICEL CHIRALCEL OD (250 mm×30 mm, 10 mm); mobile phase: [CO₂-EtOH (0.1% NH₃H₂O)]; B %:35% %, isocratic elution mode) to give 3-fluoro-4-[[5-(2-fluoro-4-methyl-anilino)-4-methyl-3-pyridyl]methyl]-2-(sulfamoylamino)pyridine (3 mg, 7.15 mmol). ¹H NMR (400 MHz, CDCl₃) δ=8.38 (s, 1H), 8.10 (s, 1H), 7.99 (d, J=5.2 Hz, 1H), 6.96-6.84 (m, 3H), 6.62 (t, J=5.2 Hz, 1H), 5.54 (brs, 2H), 5.30 (brs, 1H), 4.04 (s, 2H), 2.30 (s, 3H), 2.13 (s, 3H). ¹⁹F NMR (376.5 MHz, CDCl₃) δ=−131.914, −142.447. LCMS Rₜ=0.429 min in 1.0 min chromatography, 5-95AB, ESI calcd. for C₁₉H₂₀F₂N₅O₂S [M+H]⁺420.1, found 420.2.

### Example 97: 4-[[5-(4-cyclopropyl-2-fluoro-anilino)-4-methyl-3-pyridyl]oxy]-3-fluoro-N-(methylsulfamoyl)pyridin-2-amine

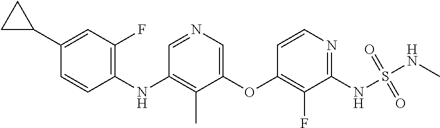 [SMILES: CNS(=O)(=O)Nc1nccc(Oc2cncc(Nc3ccc(C4CC4)cc3F)c2C)c1F]

### Route

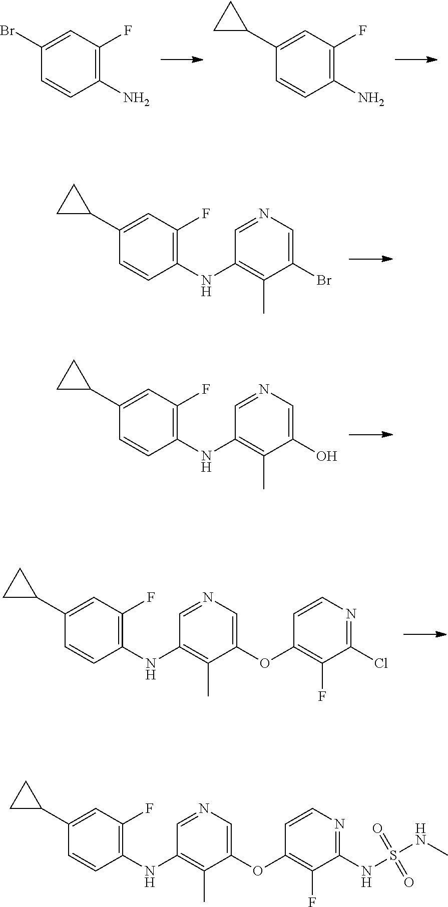 [SMILES: Nc1ccc(Br)cc1F ; Nc1ccc(C2CC2)cc1F ; Cc1c(Br)cncc1Nc1ccc(C2CC2)cc1F ; Cc1c(O)cncc1Nc1ccc(C2CC2)cc1F ; Cc1c(Nc2ccc(C3CC3)cc2F)cncc1Oc1ccnc(Cl)c1F ; CNS(=O)(=O)Nc1nccc(Oc2cncc(Nc3ccc(C4CC4)cc3F)c2C)c1F]

Step 1: To a solution of 4-bromo-2-fluoro-aniline (3 g, 15.79 mmol) in toluene (60 mL) and H₂O (5 mL) were added cyclopropylboronic acid (1.76 g, 20.52 mmol), K₃PO₄ (8.38 g, 39.47 mmol), PCy₃ (885.50 mg, 3.16 mmol, 1.02 mL) and Pd(OAc)₂ (248.12 mg, 1.11 mmol). The mixture was stirred at 110° C. for 12 h under N₂. After cooling to room temperature, the resulting mixture was filtered, and the filter cake was washed with EtOAc (50 mL×3). The filtrate was concentrated. The crude product was purified by flash chromatography on silica gel (EtOAc in Petroleum ether=0-14%) to 4-cyclopropyl-2-fluoro-aniline (3.17 g, 20.97 mmol). ¹H NMR (400 MHz, CDCl₃) δ=6.78-6.63 (m, 3H), 3.65-3.50 (m, 2H), 1.84-1.71 (m, 1H), 0.91-0.83 (m, 2H), 0.61-0.54 (m, 2H). ¹⁹F NMR (376.5 MHz, CDCl₃) δ=−135.161.

Step 2: To a solution of 4-cyclopropyl-2-fluoro-aniline (900 mg, 5.95 mmol) in dioxane (14 mL) was added 3,5-dibromo-4-methyl-pyridine (1.34 g, 5.36 mmol), Cs₂CO₃ (4.85 g, 14.88 mmol), Pd(OAc)₂ (106.92 mg, 476.25 μmol) and Xantphos (620.03 mg, 1.07 mmol). The mixture was stirred at 100° C. for 2 h under N₂. After cooling to room temperature, the resulting mixture was filtered, and the filter cake was washed with EtOAc (50 mL×3). The filtrate was concentrated. The crude product was purified by flash chromatography on silica gel (EtOAc in Petroleum ether=0-8%) to give 5-bromo-N-(4-cyclopropyl-2-fluoro-phenyl)-4-methyl-pyridin-3-amine (1.4 g, 4.36 mmol). ¹H NMR (400 MHz, CDCl₃) δ=8.35 (s, 1H), 8.26 (s, 1H), 6.94-6.75 (m, 3H), 5.41-5.21 (m, 1H), 2.37 (s, 3H), 1.90-1.80 (m, 1H), 0.99-0.90 (m, 2H), 0.68-0.60 (m, 2H). ¹⁹F NMR (376.5 MHz, CDCl₃) δ=−131.038

Step 3: To a solution of 5-bromo-N-(4-cyclopropyl-2-fluoro-phenyl)-4-methyl-pyridin-3-amine (1.35 g, 4.20 mmol) in dioxane (15 mL) were added KOH (943.28 mg, 16.81 mmol), H₂O (1.51 g, 84.06 mmol, 1.51 mL) and tbubrettphosp-pd-G3 (538.69 mg, 630.47 μmol). The mixture was stirred at 80° C. for 6 h under N₂. Water (30 mL) was added. The mixture were extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-85%) to give 5-(4-cyclopropyl-2-fluoro-anilino)-4-methyl-pyridin-3-ol (650 mg, 2.52 mmol). 1H NMR (400 MHz, DMSO-d₆) δ=9.67-9.51 (m, 1H), 7.75 (s, 1H), 7.55 (s, 1H), 7.17 (s, 1H), 6.89 (dd, J=12.8, 2.0 Hz, 1H), 6.81-6.76 (m, 1H), 6.74-6.67 (m, 1H), 1.99 (s, 3H), 1.90-1.80 (m, 1H), 0.94-0.84 (m, 2H), 0.66-0.58 (m, 2H). ¹⁹F NMR (376.5 MHz, DMSO-d₆) δ=−126.832.

Step 4: To a solution of 5-(4-cyclopropyl-2-fluoro-anilino)-4-methyl-pyridin-3-ol (600 mg, 2.32 mmol) in DMF (10 mL) were added 2-chloro-3-fluoro-4-iodo-pyridine (717.61 mg, 2.79 mmol), CuI (353.93 mg, 1.86 mmol), Cs₂CO₃ (1.14 g, 3.48 mmol) and TMHD (513.68 mg, 2.79 mmol, 573.94 μL). The mixture was stirred at 60° C. for 16 h under N₂. Water (50 mL) was added. The mixture was extracted with EtOAc (40 mL×3). The aqueous layer was washed with H₂O (40 mL×5). The combined organic layers were washed with brine (30 mL), dried over anhydrous Na₂SO₄, filtered and concentrated. The crude product was purified by flash chromatography on silica gel (EtOAc in Petroleum ether=0-45%) and Prep-HPLC (column: Phenomenex C18 80×40 mm×3 um; mobile phase: [water(NH₃H₂O+NH₄HCO₃)−ACN]; gradient: 60%-90% B over 7 min) to give 5-[(2-chloro-3-fluoro-4-pyridyl)oxy]-N-(4-cyclopropyl-2-fluoro-phenyl)-4-methyl-pyridin-3-amine (90 mg, 232.07 μmol). ¹H NMR (400 MHz, CDCl₃) δ=8.47-8.20 (m, 1H), 8.09-7.95 (m, 2H), 7.06 (t, J=8.4 Hz, 1H), 6.90-6.79 (m, 2H), 6.62 (t, J=5.6 Hz, 1H), 5.42-5.26 (m, 1H), 2.15 (s, 3H), 1.92-1.80 (m, 1H), 1.04-0.93 (m, 2H), 0.71-0.60 (m, 2H). ¹⁹F NMR (376.5 MHz, CDCl₃) δ=−129.710, −140.141.

Step 5: To a solution of 5-[(2-chloro-3-fluoro-4-pyridyl)oxy]-N-(4-cyclopropyl-2-fluoro-phenyl)-4-methyl-pyridin-3-amine (70 mg, 180.50 μmol) in dioxane (3 mL) were added (sulfamoylamino) methane (29.82 mg, 270.75 μmol), Cs₂CO₃ (176.43 mg, 541.50 μmol) and [2-(2-aminophenyl)phenyl]-methylsulfonyloxy-palladium; ditert-butyl-[3,6-dimethoxy-2-(2,4,6-triisopropylphenyl)phenyl]phosphane (77.11 mg, 90.25 μmol). The mixture was stirred at 100° C. for 1 h under N₂. The mixture was concentrated. The crude product was purified by flash chromatography on silica gel (MeOH in Dichloromethane=0-12%) and Prep-HPLC (column: Phenomenex C18 80×40 mm×3 μm; mobile phase: [water(NH₃H₂O+NH₄HCO₃)−ACN]; gradient: 40%-70% B over 7 min) to give 4-[[5-(4-cyclopropyl-2-fluoro-anilino)-4-methyl-3-pyridyl]oxy]-3-fluoro-N-(methylsulfamoyl)pyridin-2-amine (35.2 mg, 76.28 μmol). ¹H NMR (400 MHz, DMSO-d₆) δ=10.60-10.42 (m, 1H), 7.98-7.91 (m, 2H), 7.87 (d, J=2.0 Hz, 1H), 7.56 (s, 1H), 7.11-6.84 (m, 4H), 6.50-6.41 (m, 1H), 2.54 (s, 3H), 2.06 (s, 3H), 1.97-1.80 (m, 1H), 1.00-0.87 (m, 2H), 0.72-0.62 (m, 2H). ¹⁹F NMR (376.5 MHz, DMSO-d₆) δ=−124.102, −156.715. LCMS Rₜ=0.720 min in 1.5 min chromatography, 5-95AB, ESI calcd. for C₂₁H₂₂F₂N₅O₃S [M+H]⁺462.1, found 462.1.

### Example 98: 3-fluoro-4-[[5-(2-fluoro-4-methyl-phenoxy)-4-methyl-3-pyridyl]methyl]pyridin-2-amine

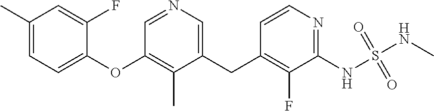

### Route

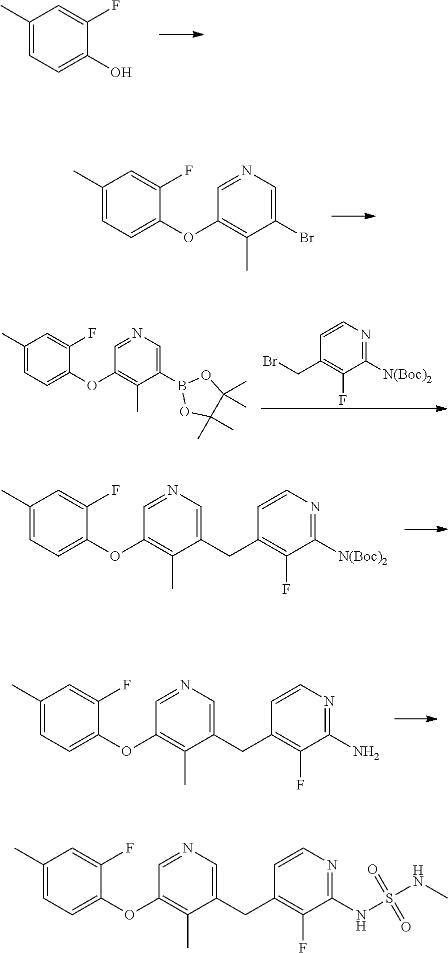

Step 1: To a solution of 3,5-dibromo-4-methyl-pyridine (5 g, 19.9 mmol) 2-fluoro-4-methyl-phenol (3.77 g, 29.9 mmol) in 50 mL NMP was added Cs₂CO₃ (19.5 g, 59.8 mmol). The mixture was stirred at 130° C. for 16 h under N₂. The reaction mixture was quenched by addition 200 mL H₂O at 25° C., and then extracted with EtOAc 300 mL (100 mL×3). The combined organic layers were washed with brine 300 mL (150 mL×2), dried over Na₂SO₄, filtered and concentrated under reduced pressure to give a residue. The residue was purified by prep-HPLC (column: YMC Triart C18 70×250 mm×7 um; mobile phase: [water (HCl)−ACN]; gradient: 32%-62% B over 25 min) to give 3-bromo-5-(2-fluoro-4-methyl-phenoxy)-4-methyl-pyridine (3.25 g, 11.0 mmol). ¹H NMR (400 MHz, CDCl₃) δ=8.41 (s, 1H), 7.93 (s, 1H), 7.03-6.97 (m, 1H), 6.93-6.85 (m, 2H), 2.43 (s, 3H), 2.34 (s, 3H). ¹⁹F NMR (376.5 MHz CDCl₃) δ=−131.675 ppmLCMS Rₜ=0.459 min in 0.8 min chromatography, 5-95AB, ESI calcd. for C₁₃H₁₂BrFNO [M+H]⁺298.0, found 297.9.

Step 2: To a solution of 3-bromo-5-(2-fluoro-4-methyl-phenoxy)-4-methyl-pyridine (3 g, 10.1 mmol), 4,4,5,5-tetramethyl-2-(4,4,5,5-tetramethyl-1,3,2-dioxaborolan-2-yl)-1,3,2-dioxaborolane (3.86 g, 15.2 mmol) in 30 mL dioxane was added Pd(dppf)Cl₂ (741.3 mg, 1.01 mmol) and KOAc (2.98 g, 30.4 mmol). The mixture was stirred at 100° C. for 16 h under N₂. After cooling to 25° C., the reaction mixture was filtered and concentrated under reduced pressure to give a residue. The residue was purified by column chromatography (SiO₂, Petroleum ether/Ethyl acetate=99/1 to 3/1) to give 3-(2-fluoro-4-methyl-phenoxy)-4-methyl-5-(4,4,5,5-tetramethyl-1,3,2-dioxaborolan-2-yl)pyridine (4.16 g, 8.48 mmol). ¹H NMR (400 MHz, CDCl₃) δ=8.62 (s, 1H), 8.10 (s, 1H), 7.01-6.96 (m, 1H), 6.86-6.82 (m, 1H), 6.78-6.72 (m, 1H), 2.50 (s, 3H), 2.32 (s, 3H), 1.36 (s, 12H) ¹⁹F NMR (376.5 MHz CDCl₃) δ=−132.648 ppm

Step 3: To a solution of 3-(2-fluoro-4-methyl-phenoxy)-4-methyl-5-(4,4,5,5-tetramethyl-1,3,2-dioxaborolan-2-yl)pyridine (1 g, 2.91 mmol), tert-butyl N-[4-(bromomethyl)-3-fluoro-2-pyridyl]-N-tert-butoxycarbonyl-carbamate (945 mg, 2.33 mmol) in a mixed solvent of 15 mL toluene, 7.5 mL EtOH and 1.5 mL H₂O was added Pd(dppf)Cl₂ (213 mg, 291 μmol) and Na₂CO₃ (926 mg, 8.74 mmol). The mixture was degassed and purged with N₂ for 3 times and stirred at 80° C. for 1 h under N₂. After cooling to 25° C., the reaction mixture was filtered and concentrated under reduced pressure and purified by column chromatography (SiO₂, Petroleum ether/Ethyl acetate=9/1 to 1/1) to give tert-butyl N-tert-butoxycarbonyl-N-[3-fluoro-4-[[5-(2-fluoro-4-methyl-phenoxy)-4-methyl-3-pyridyl]methyl]-2-pyridyl]carbamate (514 mg, 902 μmol). ¹H NMR (400 MHz CDCl₃) δ −8.19 (d, J=4.8 Hz, 1H), 8.15 (s, 1H), 8.00 (s, 1H), 7.00 (d, J=12 Hz, 1H), 6.95-6.84 (m, 3H), 4.10 (s, 2H), 2.35 (s, 3H), 2.21 (s, 3H), 1.41 (s, 18H) ¹⁹F NMR (376.5 MHz CDCl₃) δ=−131.108 ppm, −131.919 ppm. LCMS Rₜ=0.503 min in 0.8 min chromatography, 5-95AB, ESI calcd. for C₂₉H₃₄F₂N₃O₅ [M+H]⁺542.2, found 542.1.

Step 4: To a solution of tert-butyl N-tert-butoxycarbonyl-N-[3-fluoro-4-[[5-(2-fluoro-4-methyl-phenoxy)-4-methyl-3-pyridyl]methyl]-2-pyridyl]carbamate (100 mg, 185 μmol) in 2 mL dioxane was added HCl/dioxane (4 M, 3 mL). The mixture was stirred at 25° C. for 1 h under N₂. The reaction mixture was concentrated under reduced pressure to give 3-fluoro-4-[[5-(2-fluoro-4-methyl-phenoxy)-4-methyl-3-pyridyl]methyl]pyridin-2-amine (51 mg, 143 μmol). ¹H NMR (400 MHz, DMSO-d₆) δ=8.42 (s, 1H), 8.12 (s, 1H), 7.77 (d, J=6.4 Hz, 1H), 7.27 (d, J=11.2 Hz, 1H), 7.11-7.03 (m, 2H), 6.64 (t, J=6.0 Hz, 1H), 4.27 (s, 2H), 2.33 (s, 3H), 2.30 (s, 3H). ¹⁹F NMR (376.5 MHz DMSO-d₆) δ=−132.534 ppm, −137.299 ppm. LCMS Rₜ=0.303 min in 0.8 min chromatography, 5-95AB, ESI calcd. for C₁₉H₁₈F₂N₃O [M+H]⁺342.1, found 342.0.

### Example 99: 4-[[5-(4-cyclopropyl-2-fluoro-phenoxy)-4-methyl-3-pyridyl]methyl]-3-fluoro-N-(methylsulfamoyl)pyridin-2-amine

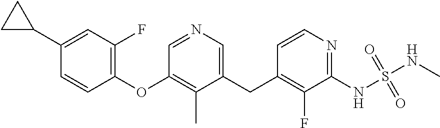 [SMILES: CNS(=O)(=O)Nc1nccc(Cc2cncc(Oc3ccc(C4CC4)cc3F)c2C)c1F]

### Route

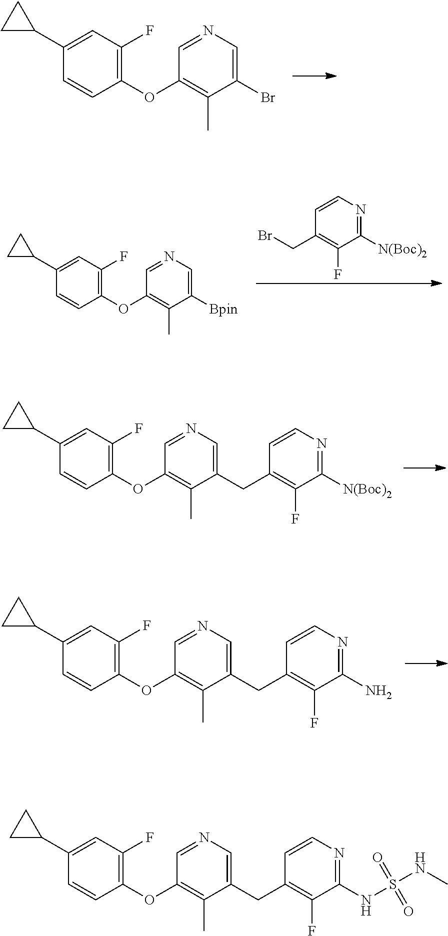

Step 1: To a solution of 3-bromo-5-(4-cyclopropyl-2-fluoro-phenoxy)-4-methyl-pyridine (650 mg, 2.02 mmol, example 102) in dioxane (8 mL) were added 4,4,5,5-tetramethyl-2-(4,4,5,5-tetramethyl-1,3,2-dioxaborolan-2-yl)-1,3,2-dioxaborolane (614.80 mg, 2.42 mmol), KOAc (594.02 mg, 6.05 mmol) and Pd(dppf)Cl₂ (147.63 mg, 201.76 μmol). The mixture was stirred at 110° C. for 12 h. After cooling to room temperature, the resulting mixture was filtered, and the filter cake was washed with EtOAc (50 mL×3). The filtrate was concentrated. The crude product was purified by flash chromatography on silica gel (EtOAc in Petroleum ether=0-11%) to give 3-(4-cyclopropyl-2-fluoro-phenoxy)-4-methyl-5-(4,4,5,5-tetramethyl-1,3,2-dioxaborolan-2-yl)pyridine (500 mg, 1.35 mmol). ¹H NMR (400 MHz, CDCl₃) δ=8.61 (s, 1H), 8.09 (s, 1H), 6.89-6.76 (m, 3H), 2.51 (s, 3H), 1.93-1.83 (m, 1H), 1.36 (s, 12H), 1.01-0.93 (m, 2H), 0.68-0.62 (m, 2H). ¹⁹F NMR (376.5 MHz, CDCl₃) δ=−132.344.

Step 2: To a solution of 3-(4-cyclopropyl-2-fluoro-phenoxy)-4-methyl-5-(4,4,5,5-tetramethyl-1,3,2-dioxaborolan-2-yl)pyridine (440 mg, 1.19 mmol) in a mixed solvent of toluene (7 mL) and H₂O (0.7 mL) were added tert-butyl N-[4-(bromomethyl)-3-fluoro-2-pyridyl]-N-tert-butoxycarbonyl-carbamate (434.63 mg, 1.07 mmol), Cs₂CO₃ (776.52 mg, 2.38 mmol) and Pd(dppf)Cl₂·CH₂Cl₂ (97.31 mg, 119.16 μmol). The mixture was stirred at 100° C. for 1 h. After cooling to room temperature, water (30 mL) was added. The mixture was extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-37%) to give tert-butyl N-tert-butoxycarbonyl-N-[4-[[5-(4-cyclopropyl-2-fluoro-phenoxy)-4-methyl-3-pyridyl]methyl]-3-fluoro-2-pyridyl]carbamate (190 mg, 334.73 μmol). ¹H NMR (400 MHz, CDCl₃) δ=8.21-8.18 (m, 1H), 8.15 (s, 1H), 7.99 (s, 1H), 6.93-6.83 (m, 4H), 4.10 (s, 2H), 2.21 (s, 3H), 1.92-1.85 (m, 1H), 1.41 (s, 18H), 1.03-0.96 (m, 2H), 0.72-0.65 (m, 2H). ¹⁹F NMR (376.5 MHz, CDCl₃) δ=−131.103, −131.684.

Step 3: To a solution of tert-butyl N-tert-butoxycarbonyl-N-[4-[[5-(4-cyclopropyl-2-fluoro-phenoxy)-4-methyl-3-pyridyl]methyl]-3-fluoro-2-pyridyl]carbamate (190 mg, 334.73 μmol) in MeOH (1 mL) was added HCl/MeOH (4 M, 4 mL, 16.00 mmol). The mixture was stirred at 25° C. for 2 h. The mixture was concentrated. 4-[[5-(4-cyclopropyl-2-fluoro-phenoxy)-4-methyl-3-pyridyl]methyl]-3-fluoro-pyridin-2-amine (130 mg, 321.90 μmol). ¹H NMR (400 MHz, CD₃OD) δ=8.53 (s, 1H), 8.17 (s, 1H), 7.73-7.65 (m, 1H), 7.27-7.21 (m, 1H), 7.11-7.03 (m, 2H), 6.80-6.70 (m, 1H), 4.48 (s, 2H), 2.61 (s, 3H), 2.04-1.91 (m, 1H), 1.10-1.00 (m, 2H), 0.78-0.68 (m, 2H). ¹⁹F NMR (376.5 MHz, CD₃OD) δ=−132.698, −137.688.

Step 4: To a solution of 4-[[5-(4-cyclopropyl-2-fluoro-phenoxy)-4-methyl-3-pyridyl]methyl]-3-fluoro-pyridin-2-amine (90 mg, 222.85 μmol, HCl) in CH₃CN (3 mL) were added N-methylsulfamoyl chloride (28.87 mg, 222.85 μmol) and Py (141.02 mg, 1.78 mmol, 143.90 μL). The mixture was stirred at 0° C. for 1 h. Then N-methylsulfamoyl chloride (57.75 mg, 445.71 μmol) was added. The mixture was stirred at 25° C. for 1 h. N-methylsulfamoyl chloride (57.75 mg, 445.71 μmol) was added. The mixture was stirred at 25° C. for 1 h. The mixture was concentrated. The crude product was purified by Prep-HPLC (column: Phenomenex C18 80×40 mm×3 um; mobile phase: [water(NH₃H₂O+NH₄HCO₃)−ACN]; gradient: 40%-70% B over 7 min) to give 4-[[5-(4-cyclopropyl-2-fluoro-phenoxy)-4-methyl-3-pyridyl]methyl]-3-fluoro-N-(methylsulfamoyl)pyridin-2-amine (35.6 mg, 77.31 μmol). ¹H NMR (400 MHz, CDCl₃) δ=8.15 (s, 1H), 8.03-7.90 (m, 2H), 7.40-7.33 (m, 1H), 6.92-6.81 (m, 3H), 6.64-6.57 (m, 1H), 5.52-5.41 (m, 1H), 4.05 (s, 2H), 2.77 (d, J=5.2 Hz, 3H), 2.23 (s, 3H), 1.93-1.81 (m, 1H), 1.05-0.93 (m, 2H), 0.72-0.62 (m, 2H). ¹⁹F NMR (376.5 MHz, CDCl₃) δ=−131.711, −142.825. LCMS Rₜ=0.739 min in 1.5 min chromatography, 5-95AB, ESI calcd. for C₂₂H₂₃F₂N₄O₃S [M+H]⁺461.1, found 461.0.

### Example 100: 4-[[5-(4-bromo-2-fluoro-phenoxy)-4-methyl-3-pyridyl]oxy]-3-fluoro-N-(methylsulfamoyl)pyridin-2-amine

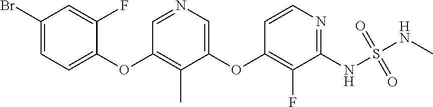 [SMILES: CNS(=O)(=O)Nc1nccc(Oc2cncc(Oc3ccc(Br)cc3F)c2C)c1F]

### Route

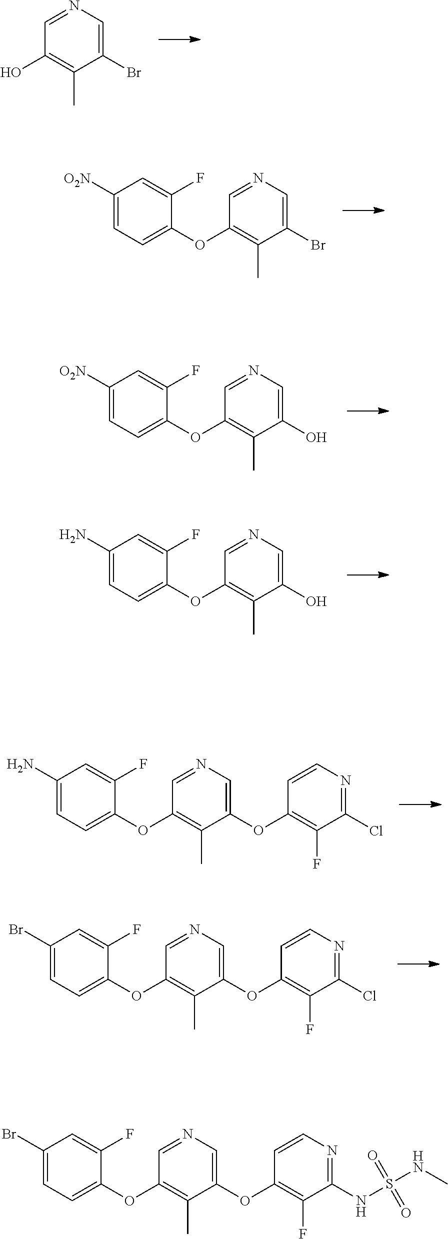

Step 1: To a solution of 5-bromo-4-methyl-pyridin-3-ol (5 g, 26.59 mmol) in MeCN (50 mL) was added KOH (1.79 g, 31.91 mmol). Then 1,2-difluoro-4-nitro-benzene (4.65 g, 29.25 mmol, 3.24 mL) was added. The mixture was stirred at 80° C. for 18 h. The mixture was concentrated. The mixture was poured into water (50 mL). The aqueous layer was extracted with EtOAc (50 mL×3). The combined organic phase was washed with brine (50 mL), dried over anhydrous Na₂SO₄, filtered and concentrated. 3-bromo-5-(2-fluoro-4-nitro-phenoxy)-4-methyl-pyridine (8.69 g, 26.57 mmol) ¹H NMR (400 MHz, CDCl₃) δ=8.61 (s, 1H), 8.17 (s, 1H), 8.13-8.02 (m, 2H), 6.86-6.92 (m, 1H), 2.35 (s, 3H).

Step 2: To a solution of 3-bromo-5-(2-fluoro-4-nitro-phenoxy)-4-methyl-pyridine (5 g, 15.29 mmol) in dioxane (50 mL) were added TBUBRETTPHOS PD G3 (1.31 g, 1.53 mmol), KOH (2.57 g, 45.86 mmol) and H₂O (5.51 g, 305.71 mmol, 5.51 mL). The mixture was stirred at 80° C. for 12 h. The mixture was concentrated under reduced pressure. The crude was purified by flash column chromatography on silica gel (EtOAc in petroleum ether=0-58%) to give 5-(2-fluoro-4-nitro-phenoxy)-4-methyl-pyridin-3-ol (2 g, 7.57 mmol) ¹H NMR (400 MHz, DMSO-d₆) δ=10.37 (s, 1H), 8.36-8.33 (m, 1H), 8.10 (s, 1H), 8.06-8.02 (m, 1H), 7.90 (s, 1H), 6.98 (t, J=8.8 Hz, 1H), 2.00 (s, 3H). ¹⁹F NMR (376.5 MHz, DMSO-d₆) δ=−130.279.

Step 3: To a solution of 5-(2-fluoro-4-nitro-phenoxy)-4-methyl-pyridin-3-ol (1.9 g, 7.19 mmol) in EtOH (18 mL) and H₂O (6 mL) were added Fe (2.01 g, 35.96 mmol) and NH₄Cl (3.85 g, 71.91 mmol). The mixture was stirred at 80° C. for 12 h. The mixture was filtered, and the filter cake was washed with MeOH (10 mL×3). The filtrate was concentrated under reduced pressure. The crude was purified by flash column chromatography on silica gel (EtOAc in petroleum ether=0-97%) to give 5-(4-amino-2-fluoro-phenoxy)-4-methyl-pyridin-3-ol (1.3 g, 5.55 mmol). ¹H NMR (400 MHz, DMSO-d₆) δ=9.95 (s, 1H), 7.86 (s, 1H), 7.38 (s, 1H), 6.87 (t, J=9.2 Hz, 1H), 6.50-6.46 (m, 1H), 6.38-6.35 (m, 1H), 5.31 (s, 2H), 2.11 (s, 3H). ¹⁹F NMR (376.5 MHz, DMSO-d₆) δ=−131.662.

Step 4: To a solution of 5-(4-amino-2-fluoro-phenoxy)-4-methyl-pyridin-3-ol (1.5 g, 6.40 mmol) in DMF (8 mL) were added 2-chloro-3-fluoro-4-iodo-pyridine (1.98 g, 7.68 mmol), CuI (60.98 mg, 320.20 μmol), TMHD (590.07 mg, 3.20 mmol, 659.30 μL) and Cs₂CO₃ (3.13 g, 9.61 mmol). The mixture was stirred at 80° C. for 2 h. Water (40 mL) was added and the mixture was extracted with EtOAc (20 mL×2). The organic layer was dried over anhydrous Na₂SO₄, filtered and concentrated. The crude was purified by flash column chromatography on silica gel (EtOAc in petroleum ether=0-50%) and then purified by Pre-HPLC (column: Xtimate C 18 150×40 mm×10 um; mobile phase: [water(NH₃H₂O+NH₄HCO₃)−ACN]; gradient: 42%-72% B over 8 min) to give 4-[[5-[(2-chloro-3-fluoro-4-pyridyl)oxy]-4-methyl-3-pyridyl]oxy]-3-fluoro-aniline (600 mg, 1.65 mmol). ¹H NMR (400 MHz, DMSO-d₆) δ=8.24 (s, 1H), 8.13 (d, J=5.6 Hz, 1H), 7.87 (s, 1H), 7.03-6.94 (m, 2H), 6.54-6.51 (m, 1H), 6.50-6.42 (m, 1H), 5.42 (s, 2H), 2.18 (s, 3H). ¹⁹F NMR (376.5 MHz, DMSO-d₆) δ=−131.407, −141.506.

Step 5: To a solution of 4-[[5-[(2-chloro-3-fluoro-4-pyridyl)oxy]-4-methyl-3-pyridyl]oxy]-3-fluoro-aniline (500 mg, 1.37 mmol) in MeCN (5 mL) were added CuBr₂ (368.42 mg, 1.65 mmol, 77.24 μL) and tert-butyl nitrite (170.10 mg, 1.65 mmol, 196.19 μL). The mixture was stirred at 60° C. for 2 h. Water (30 mL) was added and the mixture were extracted with EtOAc (20 ml×2). The organic layer was dried over anhydrous Na₂SO₄, filtered and concentrated. The crude was purified by flash column chromatography on silica gel (EtOAc in petroleum ether=0-25%) to give 3-(4-bromo-2-fluoro-phenoxy)-5-[(2-chloro-3-fluoro-4-pyridyl)oxy]-4-methyl-pyridine (320 mg, 748.32 μmol). ¹H NMR (400 MHz, DMSO-d₆) δ=8.40 (s, 1H), 8.21 (s, 1H), 8.14 (d, J=5.6 Hz, 1H), 7.80-7.77 (m, 1H), 7.42 (d, J=8.8 Hz, 1H), 7.18 (t, J=8.8 Hz, 1H), 7.04 (t, J=5.6 Hz, 1H), 2.14 (s, 3H). ¹⁹F NMR (376.5 MHz, DMSO-d₆) δ=−129.237, −141.416 ppm.

Step 6: To a solution of 3-(4-bromo-2-fluoro-phenoxy)-5-[(2-chloro-3-fluoro-4-pyridyl)oxy]-4-methyl-pyridine (180 mg, 420.93 μmol) in dioxane (2 mL) were added (sulfamoylamino) methane (60.27 mg, 547.21 μmol), [2-(2-aminophenyl)phenyl]-methylsulfonyloxy-palladium; ditert-butyl-[3,6-dimethoxy-2-(2,4,6-triisopropylphenyl)phenyl]phosphane (179.83 mg, 210.46 μmol) and Cs₂CO₃ (411.44 mg, 1.26 mmol). The mixture was stirred at 60° C. for 1 h. Water (20 mL) was added and the mixture were extracted with EtOAc (20 ml×2). The organic layer was dried over anhydrous Na₂SO₄, filtered and concentrated. The crude was purified by flash column chromatography on silica gel (EtOAc in petroleum ether=0-40%) and then purified by Prep-HPLC (column: Phenomenex C 18 80×40 mm×3 □m; mobile phase: [water(NH₃H₂O+NH₄HCO₃)−ACN]; gradient: 30%-60% B over 7 min) to give 4-[[5-(4-bromo-2-fluoro-phenoxy)-4-methyl-3-pyridyl]oxy]-3-fluoro-N-(methylsulfamoyl)pyridin-2-amine (1 mg, 1.99 μmol). ¹H NMR (400 MHz, CDCl₃) δ=8.15 (s, 1H), 8.00-7.95 (m, 2H), 7.43-7.04 (m, 1H), 7.32 (d, J=8.8 Hz, 1H), 6.99 (t, J=8.4 Hz, 1H), 6.42 (t, J=6.0 Hz, 1H), 5.47 (s, 1H), 2.81 (s, 3H), 2.29 (s, 3H). ¹⁹F NMR (376.5 MHz, CDCl₃) δ=−127.257, −159.298. LCMS Rₜ=0.883 min in 1.5 min chromatography, 5-95AB, ESI calcd. for C₁₈H₁₆BrF₂N₄O₄S [M+H]⁺502.0, found 502.7.

### Example 101: 3-fluoro-4-[[5-[2-fluoro-4-(trideuteriomethyl) phenoxy]-4-methyl-3-pyridyl]methyl]-N-(methylsulfamoyl)pyridin-2-amine

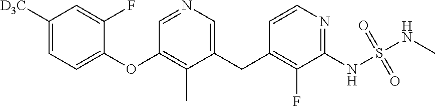 [SMILES: [2H]C([2H])([2H])c1ccc(Oc2cncc(Cc3ccnc(NS(=O)(=O)NC)c3F)c2C)c(F)c1]

### Route

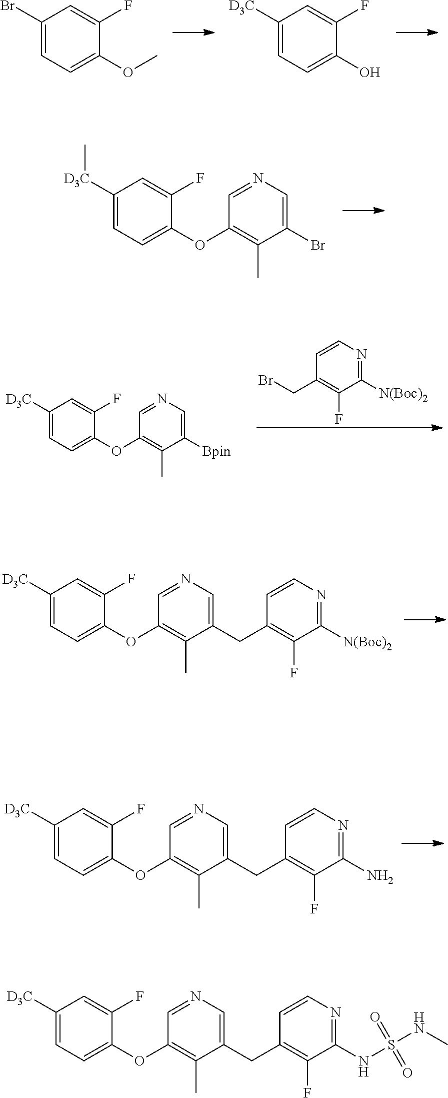

Step 1: A 250 mL three-necked round bottom flask equipped with thermometer was charged with a solution of 4-bromo-2-fluoro-1-methoxy-benzene (10 g, 48.77 mmol) in THF (80 mL). The flask was degassed and purged with N₂ for 3 times. Then n-BuLi (2.5 M in heaxanes, 21.46 mL) was added dropwise under N₂ at −70° C. The resulting mixture was stirred at −70° C. for 30 mins. Then a solution of trideuterio (iodo) methane (7.78 g, 53.65 mmol, 3.34 mL) in THF (10 mL) was added dropwise under N₂ at −70° C. The mixture was stirred at −70° C. for 1 h under N₂. The mixture was quenched with sat. NH₄Cl (300 mL), extracted with EtOAc (100 mL×2). The combined organic layers were washed with brine (200 mL), dried over anhydrous Na₂SO₄, filtered and concentrated. The residue was purified by flash column chromatography on silica gel (Ethyl acetate in Petroleum ether=0%) to give 2-fluoro-1-methoxy-4-(trideuteriomethyl)benzene (6.2 g, 43.30 mmol). ¹H NMR (400 MHz, CDCl₃) δ=6.93-6.81 (m, 3H), 3.86 (s, 3H). ¹⁹F NMR (376.5 MHz, CDCl₃) δ=−136.143.

Step 2: To a solution of 2-fluoro-1-methoxy-4-(trideuteriomethyl)benzene (6 g, 41.91 mmol) in DCM (40 mL) was added BBr₃ (21.00 g, 83.81 mmol, 8.08 mL) dropwise at 0° C. under N₂. The mixture was stirred at 25° C. for 1 h. The mixture was added dropwise into H₂O (50 mL). The mixture was adjusted to pH=7 with saturated NaHCO₃ solution slowly and the aqueous layer was extracted with DCM (50 mL×2). The combined organic layers were washed with brine (100 mL), dried over anhydrous Na₂SO₄, filtered and concentrated to give 2-fluoro-4-(trideuteriomethyl) phenol (5.41 g, 41.89 mmol). ¹H NMR (400 MHz, CDCl₃) δ=6.93-6.80 (m, 3H), 4.91 (s, 1H). ¹⁹F NMR (376.5 MHz, CDCl₃) δ=−141.660.

Step 3: To a solution of 2-fluoro-1-methoxy-4-(trideuteriomethyl)benzene (6 g, 41.91 mmol) in DCM (40 mL) was added BBr₃ (21.00 g, 83.81 mmol, 8.08 mL) dropwise at 0° C. under N₂. The mixture was stirred at 25° C. for 1 h. The mixture was added dropwise into H₂O (50 mL). The mixture was adjusted to pH=7 with saturated NaHCO₃ solution slowly and the aqueous layer was extracted with DCM (50 mL×2). The combined organic layers were washed with brine (100 mL), dried over anhydrous Na₂SO₄, filtered and concentrated to give 2-fluoro-4-(trideuteriomethyl) phenol (5.41 g, 41.89 mmol). ¹H NMR (400 MHz, CDCl₃) δ=6.93-6.80 (m, 3H), 4.91 (s, 1H). ¹⁹F NMR (376.5 MHz, CDCl₃) δ=−141.660.

Step 4: A mixture of 3-bromo-5-[2-fluoro-4-(trideuteriomethyl) phenoxy]-4-methyl-pyridine (3.4 g, 11.37 mmol), cyclopentyl(diphenyl) phosphane; dichloropalladium; iron (831.61 mg, 1.14 mmol), 4,4,5,5-tetramethyl-2-(4,4,5,5-tetramethyl-1,3,2-dioxaborolan-2-yl)-1,3,2-dioxaborolane (4.33 g, 17.05 mmol) and KOAc (3.35 g, 34.10 mmol) in dioxane (30 mL) was stirred at 110° C. for 2 h under N₂. The mixture was filtered and the filter cake was washed with EtOAc (20 mL). The filtrate was concentrated. The residue was purified by flash chromatography on silica gel (Ethyl acetate in Petroleum ether=0-60%) to give 3-[2-fluoro-4-(trideuteriomethyl) phenoxy]-4-methyl-5-(4,4,5,5-tetramethyl-1,3,2-dioxaborolan-2-yl)pyridine (3.2 g, 9.24 mmol). ¹H NMR (400 MHz, CDCl₃) δ=8.62 (s, 1H), 8.08 (s, 1H), 6.99 (d, J=11.6 Hz, 1H), 6.87 (d, J=8.4 Hz, 1H), 6.82-6.76 (m, 1H), 2.54 (s, 3H), 1.36 (s, 12H). ¹⁹F NMR (376.5 MHz, CDCl₃) δ=−132.090.

Step 5: A mixture of 3-[2-fluoro-4-(trideuteriomethyl) phenoxy]-4-methyl-5-(4,4,5,5-tetramethyl-1,3,2-dioxaborolan-2-yl)pyridine (3.2 g, 9.24 mmol), tert-butyl N-[4-(bromomethyl)-3-fluoro-2-pyridyl]-N-tert-butoxycarbonyl-carbamate (3.66 g, 8.32 mmol), Na₂CO₃ (2.94 g, 27.73 mmol) and Pd(PPh₃)₄ (1.07 g, 924.27 μmol) in toluene (32 mL) EtOH (8 mL) and H₂O (2 mL) was stirred at 80° C. for 2 h under N₂. The mixture was concentrated. The residue was purified by flash chromatography on silica gel (Ethyl acetate in Petroleum ether=0-60%) to give tert-butyl N-tert-butoxycarbonyl-N-[3-fluoro-4-[[5-[2-fluoro-4-(trideuteriomethyl) phenoxy]-4-methyl-3-pyridyl]methyl]-2-pyridyl]carbamate (1.7 g, 3.12 mmol). ¹H NMR (400 MHz, CDCl₃) δ=8.22-8.15 (m, 2H), 7.99 (br s, 1H), 7.04-6.86 (m, 4H), 4.10 (s, 2H), 2.22 (s, 3H), 1.41 (s, 18H). ¹⁹F NMR (376.5 MHz, CDCl₃) δ=−131.075, −131.877.

Step 6: A solution of tert-butyl N-tert-butoxycarbonyl-N-[3-fluoro-4-[[5-[2-fluoro-4-(trideuteriomethyl) phenoxy]-4-methyl-3-pyridyl]methyl]-2-pyridyl]carbamate (1.7 g, 3.12 mmol) in HCl/MeOH (15 mL) was stirred at 20° C. for 12 h. The mixture was concentrated. NH₃/MeOH (7 M, 20 mL) was added and then concentrated. The residue was purified by flash chromatography on silica gel (Ethyl acetate in Petroleum ether=0-100%) to give 3-fluoro-4-[[5-[2-fluoro-4-(trideuteriomethyl) phenoxy]-4-methyl-3-pyridyl]methyl]pyridin-2-amine (500 mg, 1.45 mmol). ¹H NMR (400 MHz, DMSO-d₆) δ=8.19 (s, 1H), 7.93 (s, 1H), 7.64 (d, J=4.8 Hz, 1H), 7.22 (dd, J=2.0, 12.0 Hz, 1H), 7.08-6.86 (m, 2H), 6.22 (t, J=4.8 Hz, 1H), 6.14 (s, 2H), 4.02 (s, 2H), 2.17 (s, 3H). ¹⁹F NMR (376.5 MHz, DMSO-d₆) δ=−133.036, −144.948.

Step 7: To a solution of 3-fluoro-4-[[5-[2-fluoro-4-(trideuteriomethyl) phenoxy]-4-methyl-3-pyridyl]methyl]pyridin-2-amine (100 mg, 290.38 μmol) and Py (114.85 mg, 1.45 mmol, 117.19 μL) in MeCN (2 mL) was added N-methylsulfamoyl chloride (56.44 mg, 435.57 umol) under N₂. The mixture was stirred at 25° C. for 1 h. N—N-methylsulfamoyl chloride (75.25 mg, 580.77 μmol) was added. The mixture was stirred at 25° C. for 1 h. The mixture was concentrated. The residue was purified by flash chromatography on silica gel (MeOH in DCM=0-10%) and then purified by prep-HPLC (column: Boston Prime C18 150*30 mm*5 μm; mobile phase: [water (0.05% NH₃H₂O 10 mM NH₄HCO₃)−ACN]; gradient: 33%-63% B over 7 min) to give 3-fluoro-4-[[5-[2-fluoro-4-(trideuteriomethyl) phenoxy]-4-methyl-3-pyridyl]methyl]-N-(methylsulfamoyl)pyridin-2-amine (18 mg, 41.14 μmol). ¹H NMR (400 MHz, DMSO-d₆) δ=8.18 (s, 1H), 7.93 (s, 1H), 7.80 (d, J=4.8 Hz, 1H), 7.50-7.03 (m, 3H), 7.02-6.91 (m, 2H), 6.43 (t, J=4.8 Hz, 1H), 4.04 (s, 2H), 2.41 (s, 3H), 2.17 (s, 3H). ¹⁹F NMR (376.5 MHz, DMSO-d₆) δ=−133.058, −138.878. LCMS Rₜ=0.506 min in 1 min chromatography, 5-95AB, ESI calcd. for C₂₀H₁₈D₃F₂N₄O₃S [M+H]⁺438.1, found 438.3.

### Example 102: 4-[[5-(4-cyclopropyl-2-fluoro-phenoxy)-4-methyl-3-pyridyl]oxy]-3-fluoro-N-(methylsulfamoyl)pyridin-2-amine

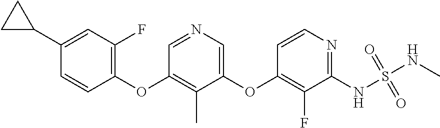 [SMILES: CNS(=O)(=O)Nc1nccc(Oc2cncc(Oc3ccc(C4CC4)cc3F)c2C)c1F]

### Route

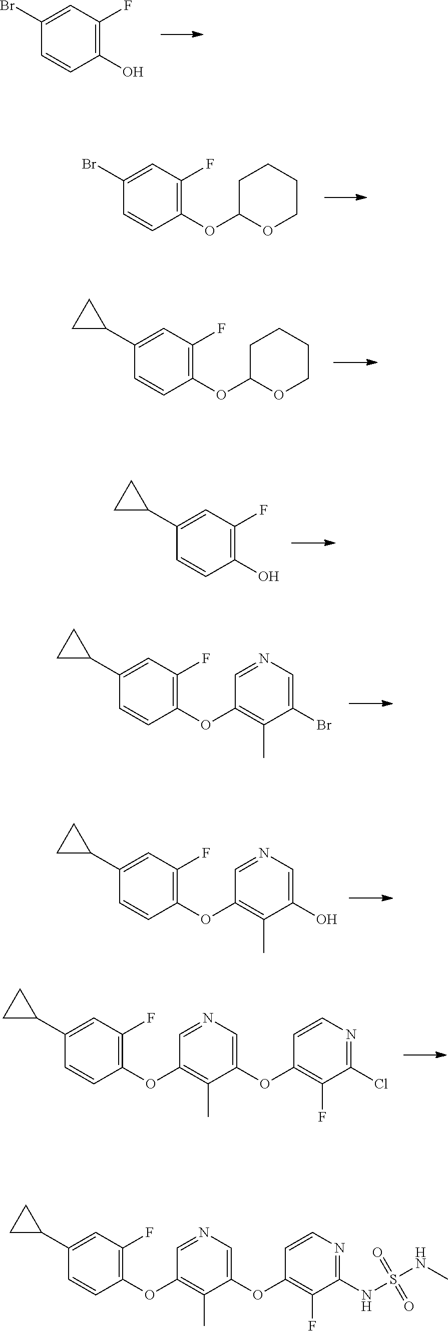

Step 1: To a solution of 4-bromo-2-fluoro-phenol (10 g, 52.36 mmol) and 4-methylbenzenesulfonic acid; pyridine (1.32 g, 5.24 mmol) in DCM (100 mL) was added 3,4-dihydro-2H-pyran (5.28 g, 62.83 mmol, 5.74 mL) at 0° C. The mixture was stirred at 25° C. for 12 h. Water (30 mL) was added. The mixture were extracted with DCM (50 mL×3). The combined organic layers were washed with brine (30 mL), dried over anhydrous Na₂SO₄, filtered and concentrated. The crude product was purified by flash chromatography on silica gel (EtOAc in Petroleum ether=0-9%) to give 2-(4-bromo-2-fluoro-phenoxy)tetrahydropyran (11.7 g, 42.53 mmol). ¹H NMR (400 MHz, CDCl₃) δ=7.23-7.09 (m, 2H), 7.07-6.99 (m, 1H), 5.36 (t, J=2.8 Hz, 1H), 3.90-3.76 (m, 1H), 3.62-3.50 (m, 1H), 1.99-1.79 (m, 3H), 1.72-1.50 (m, 3H). ¹⁹F NMR (376.5 MHz, CDCl₃) δ=−130.199.

Step 2: To a solution of 2-(4-bromo-2-fluoro-phenoxy)tetrahydropyran (5 g, 18.17 mmol) in toluene (100 mL) and H₂O (8 mL) were added cyclopropylboronic acid (1.87 g, 21.81 mmol), K₃PO₄ (9.64 g, 45.44 mmol), PCy₃ (3.06 g, 10.90 mmol, 3.54 mL) and Pd(OAc)₂ (1.22 g, 5.45 mmol). The mixture was stirred at 110° C. for 12 h. After cooling to room temperature, the resulting mixture was filtered, and the filter cake was washed with EtOAc (50 mL×3). The filtrate was concentrated. The crude product was purified by flash chromatography on silica gel (EtOAc in Petroleum ether=0-5%) to give 2-(4-cyclopropyl-2-fluoro-phenoxy)tetrahydropyran (4.29 g, 18.16 mmol). ¹H NMR (400 MHz, CDCl₃) δ=7.07 (t, J=8.4 Hz, 1H), 6.82-6.70 (m, 2H), 4.02-3.92 (m, 1H), 3.65-3.53 (m, 1H), 2.10-1.90 (m, 4H), 1.30-1.01 (m, 4H), 0.97-0.86 (m, 2H), 0.70-0.54 (m, 2H). ¹⁹F NMR (376.5 MHz, CDCl₃) δ=−133.690.

Step 3: To a solution of 2-(4-cyclopropyl-2-fluoro-phenoxy)tetrahydropyran (4.29 g, 18.16 mmol) in MeOH (5 mL) was added HCl/MeOH (4 M, 12.38 mL, 49.50 mmol). The mixture was stirred at 25° C. for 12 h. The mixture was concentrated. The crude product was purified by flash chromatography on silica gel (EtOAc in Petroleum ether=0-5%) to give 4-cyclopropyl-2-fluoro-phenol (2 g, 13.14 mmol). ¹H NMR (400 MHz, CDCl₃) δ=6.92-6.83 (m, 1H), 6.81-6.70 (m, 2H), 5.72-4.26 (m, 1H), 1.88-1.74 (m, 1H), 0.96-0.83 (m, 2H), 0.65-0.51 (m, 2H). ¹⁹F NMR (376.5 MHz, CDCl₃) δ=−141.208.

Step 4: To a solution of 4-cyclopropyl-2-fluoro-phenol (2 g, 13.14 mmol) in NMP (20 mL) were added 3,5-dibromo-4-methyl-pyridine (3.96 g, 15.77 mmol) and Cs₂CO₃ (12.85 g, 39.43 mmol). The mixture was stirred at 145° C. for 12 h. Water (50 mL) was added. The mixture was extracted with EtOAc (40 mL×3). The aqueous layer were washed with H₂O (40 mL×5). The combined organic layers were washed with brine (30 mL), dried over anhydrous Na₂SO₄, filtered and concentrated. The crude product was purified by flash chromatography on silica gel (EtOAc in Petroleum ether=0-5%) to give 3-bromo-5-(4-cyclopropyl-2-fluoro-phenoxy)-4-methyl-pyridine (4.2 g, 13.04 mmol). ¹H NMR (400 MHz, CDCl₃) δ=8.51-8.31 (m, 1H), 8.09-7.84 (m, 1H), 6.99-6.80 (m, 3H), 2.42 (s, 3H), 1.94-1.82 (m, 1H), 1.10-0.96 (m, 2H), 0.74-0.65 (m, 2H). ¹⁹F NMR (376.5 MHz, CDCl₃) δ=−131.459.

Step 5: To a solution of 3-bromo-5-(4-cyclopropyl-2-fluoro-phenoxy)-4-methyl-pyridine (2 g, 6.21 mmol) in dioxane (20 mL) were added TBUBRETTPHOS PD G3 (265.21 mg, 310.39 umol), KOH (1.04 g, 18.62 mmol), H₂O (2.24 g, 124.16 mmol, 2.24 mL), ditert-butyl-[3,6-dimethoxy-2-(2,4,6-triisopropylphenyl)phenyl]phosphane (150.44 mg, 310.39 μmol). Then the reaction was stirred at 80° C. for 3 h. The mixture was concentrated. The crude product was purified by flash chromatography on silica gel (EtOAc in Petroleum ether=0-12%) to give 5-(4-cyclopropyl-2-fluoro-phenoxy)-4-methyl-pyridin-3-ol (900 mg, 3.47 mmol). 1H NMR (400 MHz, CDCl₃) δ=8.02 (s, 1H), 7.54 (s, 1H), 6.95-6.80 (m, 3H), 2.28 (s, 3H), 1.95-1.84 (m, 1H), 1.09-0.94 (m, 2H), 0.74-0.64 (m, 2H). ¹⁹F NMR (376.5 MHz, CDCl₃) δ=−131.515.

Step 6: To a solution of 5-(4-cyclopropyl-2-fluoro-phenoxy)-4-methyl-pyridin-3-ol (800 mg, 3.09 mmol) in DMF (15 mL) were added 2-chloro-3-fluoro-4-iodo-pyridine (953.17 mg, 3.70 mmol), CuI (587.64 mg, 3.09 mmol), Cs₂CO₃ (2.01 g, 6.17 mmol) and TMHD (1.14 g, 6.17 mmol, 1.27 mL). The mixture was stirred at 60° C. for 12 h. Then 2-chloro-3-fluoro-4-iodo-pyridine (1.99 g, 7.71 mmol), CuI (1.18 g, 6.17 mmol), Cs₂CO₃ (4.02 g, 12.34 mmol) and TMHD (2.27 g, 12.34 mmol, 2.54 mL) was added. The mixture was stirred at 60° C. for 16 h. Water (50 mL) was added. The mixture was extracted with EtOAc (40 mL×3). The aqueous layer were washed with H₂O (40 mL×5). The combined organic layers were washed with brine (30 mL), dried over anhydrous Na₂SO₄, filtered and concentrated. The crude product was purified by flash chromatography on silica gel (EtOAc in Petroleum ether=0-45%) and Prep-HPLC (column: Phenomenex C18 80×40 mm×3 μm; mobile phase: [water(NH₃H₂O+NH₄HCO₃)−ACN]; gradient: 65%-95% B over 7 min) to give 2-chloro-4-[[5-(4-cyclopropyl-2-fluoro-phenoxy)-4-methyl-3-pyridyl]oxy]-3-fluoro-pyridine (280 mg, 720.17 μmol). ¹H NMR (400 MHz, CDCl₃) δ=8.12 (s, 1H), 8.06 (dd, J=5.6, 0.8 Hz, 1H), 7.98 (s, 1H), 6.99 (t, J=8.4 Hz, 1H), 6.94-6.86 (m, 2H), 6.62 (t, J=5.6 Hz, 1H), 2.26 (s, 3H), 2.01-1.83 (m, 1H), 1.07-0.97 (m, 2H), 0.73-0.65 (m, 2H). ¹⁹F NMR (376.5 MHz, CDCl₃) δ=−131.057, −139.809.

Step 7: To a solution of 2-chloro-4-[[5-(4-cyclopropyl-2-fluoro-phenoxy)-4-methyl-3-pyridyl]oxy]-3-fluoro-pyridine (120 mg, 308.65 μmol) in dioxane (1 mL) were added (sulfamoylamino) methane (50.99 mg, 462.97 μmol), TBUBRETTPHOS PD G3 (131.86 mg, 154.32 μmol) and Cs₂CO₃ (301.69 mg, 925.94 μmol). The mixture was stirred at 100° C. for 1 h. The mixture was blended with another batch prepared form 120 mg of KEY INT 7. The mixture was concentrated. The crude product was purified by flash chromatography on silica gel (MeOH in DCM=0-14%) and Prep-HPLC (column: Phenomenex C18 80×30 mm×5 μm; mobile phase: [water(NH₃H₂O+NH₄HCO₃)−ACN]; gradient: 30%-60% B over 7 min) to give 4-[[5-(4-cyclopropyl-2-fluoro-phenoxy)-4-methyl-3-pyridyl]oxy]-3-fluoro-N-(methylsulfamoyl)pyridin-2-amine (114.3 mg, 247.15 μmol). ¹H NMR (400 MHz, CDCl₃) δ=8.11 (s, 1H), 8.00-7.85 (m, 2H), 6.99 (t, J=8.0 Hz, 1H), 6.91-6.84 (m, 2H), 6.36 (d, J=6.0 Hz, 1H), 5.58-5.40 (m, 1H), 2.80 (s, 3H), 2.25 (s, 3H), 1.95-1.85 (m, 1H), 1.07-0.96 (m, 2H), 0.75-0.62 (m, 2H). ¹⁹F NMR (376.5 MHz, CDCl₃) δ=−131.112, −159.999. LCMS Rₜ=0.654 min in 1.5 min chromatography, 5-95AB, ESI calcd. for C₂₁H₂₁F₂N₄O₄S [M+H]⁺463.1, found 463.2.

### Example 103: 3-fluoro-4-[[5-(3-fluoro-4-methyl-phenoxy)-4-methyl-3-pyridyl]methyl]-N-(methylsulfamoyl)pyridin-2-amine

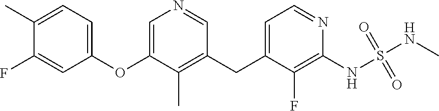 [SMILES: CNS(=O)(=O)Nc1nccc(Cc2cncc(Oc3ccc(C)c(F)c3)c2C)c1F]

### Route

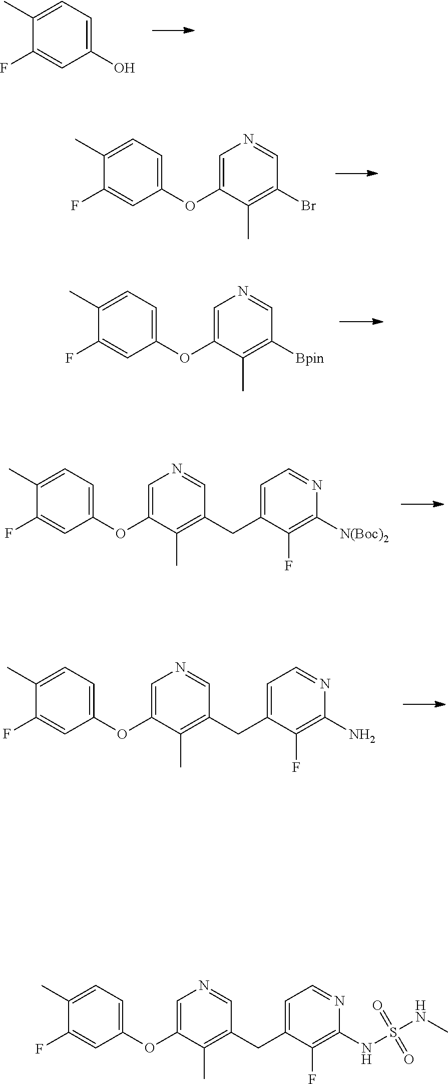

Step 1: To a solution of 3-fluoro-4-methyl-phenol (2 g, 15.86 mmol) in NMP (20 mL) were added 3,5-dibromo-4-methyl-pyridine (5.17 g, 20.61 mmol) and Cs₂CO₃ (15.50 g, 47.57 mmol). The mixture was stirred at 145° C. for 2 h. Water (30 mL) was added. The mixture were extracted with EtOAc (50 mL×3). The combined organic layers were washed with brine (30 mL), dried over anhydrous Na₂SO₄, filtered and concentrated. The crude product was purified by flash chromatography on silica gel (EtOAc in Petroleum ether=0-3%) to give 3-bromo-5-(3-fluoro-4-methyl-phenoxy)-4-methyl-pyridine (4.70 g, 15.87 mmol). ¹H NMR (400 MHz, CDCl₃) δ=8.49 (s, 1H), 8.11 (s, 1H), 7.16-7.08 (m, 1H), 6.65-6.56 (m, 2H), 2.33 (s, 3H), 2.26-2.21 (m, 3H). ¹⁹F NMR (376.5 MHz, CDCl₃) δ=−113.569.

Step 2: To a solution of 3-bromo-5-(3-fluoro-4-methyl-phenoxy)-4-methyl-pyridine (1 g, 3.38 mmol) in dioxane (13 mL) were added 4,4,5,5-tetramethyl-2-(4,4,5,5-tetramethyl-1,3,2-dioxaborolan-2-yl)-1,3,2-dioxaborolane (1.03 g, 4.05 mmol), XPhos (321.96 mg, 675.37 μmol), KOAc (994.23 mg, 10.13 mmol) and Pd(OAc)₂ (75.81 mg, 337.68 μmol). The mixture was stirred at 110° C. for 0.5 h. Then Pd(OAc)₂ (75.81 mg, 337.68 μmol) was added. The mixture was stirred at 110° C. for 1 h. Then a mixture of tert-butyl N-[4-(bromomethyl)-3-fluoro-2-pyridyl]-N-tert-butoxycarbonyl-carbamate (1.06 g, 2.62 mmol), Cs₂CO₃ (1.90 g, 5.83 mmol) and Pd(dppf)Cl₂·CH₂Cl₂ (237.95 mg, 291.38 μmol) in toluene (10 mL) and H₂O (1 mL) was added. The mixture was stirred at 100° C. for 1 h. Water (40 mL) was added. The mixture were extracted with EtOAc (40 mL×3). The combined organic layers were washed with brine (30 mL), dried over anhydrous Na₂SO₄, filtered and concentrated. The crude product was purified by flash chromatography on silica gel (EtOAc in Petroleum ether=0-52%) to give tert-butyl N-tert-butoxycarbonyl-N-[3-fluoro-4-[[5-(3-fluoro-4-methyl-phenoxy)-4-methyl-3-pyridyl]methyl]-2-pyridyl]carbamate (530 mg, 978.61 μmol). ¹H NMR (400 MHz, CDCl₃) δ=8.25-8.10 (m, 3H), 7.19-7.09 (m, 1H), 6.96-6.90 (m, 1H), 6.64-6.55 (m, 2H), 4.13-4.11 (m, 2H), 2.25-2.23 (m, 3H), 2.18 (s, 3H), 1.42 (s, 18H). ¹⁹F NMR (376.5 MHz, CDCl₃) δ=−130.916, −132.505.

Step 3: To a solution of tert-butyl N-tert-butoxycarbonyl-N-[3-fluoro-4-[[5-(3-fluoro-4-methyl-phenoxy)-4-methyl-3-pyridyl]methyl]-2-pyridyl]carbamate (530 mg, 978.61 μmol) in MeOH (1 mL) was added HCl/MeOH (4 M, 5 mL, 20.00 mmol). The mixture was stirred at 25° C. for 14 h. The mixture was concentrated. 3-fluoro-4-[[5-(3-fluoro-4-methyl-phenoxy)-4-methyl-3-pyridyl]methyl]pyridin-2-amine (300 mg, 794.04). ¹H NMR (400 MHz, CD₃OD) δ=8.56 (s, 1H), 8.30 (s, 1H), 7.77-7.67 (m, 1H), 7.42-7.30 (m, 1H), 7.09-6.91 (m, 2H), 6.82-6.72 (m, 1H), 4.47 (s, 2H), 2.54 (s, 3H), 2.28 (s, 3H). ¹⁹F NMR (376.5 MHz, CD₃OD) δ=−114.458, −137.692.

Step 4: To a solution of 3-fluoro-4-[[5-(3-fluoro-4-methyl-phenoxy)-4-methyl-3-pyridyl]methyl]pyridin-2-amine (120 mg, 351.54 μmol) in CH₃CN (1.5 mL) were added N-methylsulfamoyl chloride (68.32 mg, 527.31 μmol) and Py (194.65 mg, 2.46 mmol, 198.62 μL). The mixture was stirred at 25° C. for 2 h. The mixture was concentrated. The crude product was purified by prep-HPLC (column: Phenomenex C18 80×40 mm×3 μm; mobile phase: [water(NH₃H₂O+NH₄HCO₃)−ACN]; gradient: 32%-62% B over 7 min) to give 3-fluoro-4-[[5-(3-fluoro-4-methyl-phenoxy)-4-methyl-3-pyridyl]methyl]-N-(methylsulfamoyl)pyridin-2-amine (14 mg, 32.22 μmol). ¹H NMR (400 MHz, CDCl₃) δ=8.27-8.10 (m, 2H), 7.97 (d, J=4.8 Hz, 1H), 7.12 (t, J=8.8 Hz, 1H), 6.68-6.50 (m, 3H), 5.55-5.35 (m, 1H), 4.05 (s, 2H), 2.77 (d, J=4.8 Hz, 3H), 2.23 (s, 3H), 2.14 (s, 3H). ¹⁹F NMR (376.5 MHz, CDCl₃) δ=−113.767, −142.781. LCMS Rₜ=0.808 min in 1.5 min chromatography, 5-95AB, ESI calcd. for C₂₀H₂₁F₂N₄O₃S [M+H]⁺435.1, found 435.1.

### Example 104: 3-fluoro-4-[[5-[2-fluoro-4-(trideuteriomethyl) anilino]-4-methyl-3-pyridyl]methyl]-N-(methylsulfamoyl)pyridin-2-amine

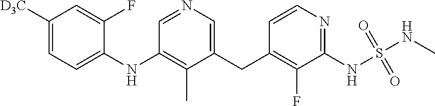 [SMILES: [2H]C([2H])([2H])c1ccc(Nc2cncc(Cc3ccnc(NS(=O)(=O)NC)c3F)c2C)c(F)c1]

### Route

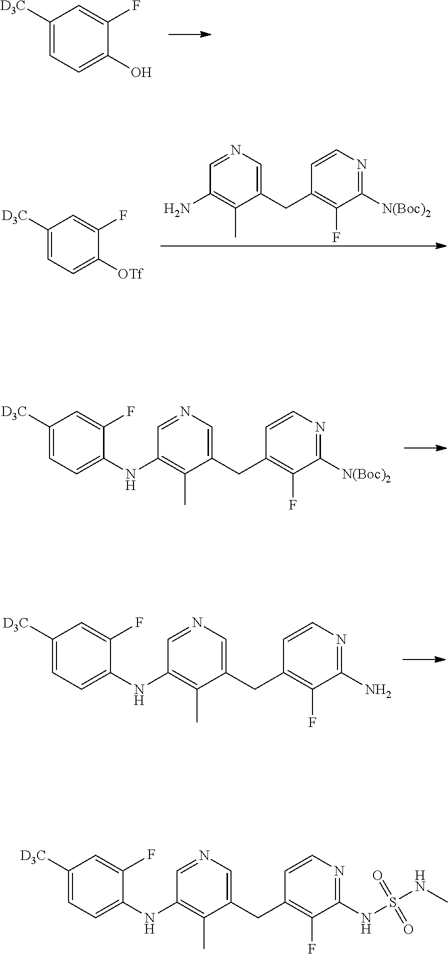

Step 1: To a solution of 2-fluoro-4-(trideuteriomethyl) phenol (2.6 g, 20.13 mmol) and TEA (5.09 g, 50.33 mmol, 7.01 mL) in DCM (20 mL) was added trifluoromethylsulfonyl trifluoromethanesulfonate (11.36 g, 40.26 mmol, 6.64 mL) dropwise at 0° C. under N₂. The mixture was stirred at 25° C. for 2 h. The mixture was washed with sat.NaHCO₃ (50 mL), 1N HCl (50 mL) and brine (100 mL). The combined organic layers were dried over anhydrous Na₂SO₄, filtered and concentrated. The residue was purified by flash column chromatography on silica gel (Ethyl acetate in Petroleum ether=0%) to give [2-fluoro-4-(trideuteriomethyl)phenyl] trifluoromethanesulfonate (3.2 g, 12.25 mmol). ¹H NMR (400 MHz, CDCl₃) δ=7.20 (t, J=8.0 Hz, 1H), 7.07 (dd, J=2.0, 10.4 Hz, 1H), 7.02-6.97 (m, 1H). ¹⁹F NMR (376.5 MHz, CDCl₃) δ=−73.264, −128.400.

Step 2: To a solution of [2-fluoro-4-(trideuteriomethyl)phenyl] trifluoromethanesulfonate (434.86 mg, 1.66 mmol), tert-butyl N-[4-[(5-amino-4-methyl-3-pyridyl)methyl]-3-fluoro-2-pyridyl]-N-tert-butoxycarbonyl-carbamate (400 mg, 924.88 μmol) and Cs₂CO₃ (904.03 mg, 2.77 mmol) in dioxane (7 mL) were added Pd₂(dba)₃ (84.69 mg, 92.49 μmol) and Xantphos (53.52 mg, 92.49 μmol) under N₂. The mixture was stirred at 110° C. for 2 h. The mixture was concentrated. The residue was purified by flash chromatography on silica gel (Ethyl acetate in Petroleum ether=0-100%) to give tert-butyl N-tert-butoxycarbonyl-N-[3-fluoro-4-[[5-[2-fluoro-4-(trideuteriomethyl) anilino]-4-methyl-3-pyridyl]methyl]-2-pyridyl]carbamate (300 mg, 551.86 μmol). LCMS Rₜ=0.577 min in 1 min chromatography, 5-95AB, ESI calcd. for C₂₉H₃₂D₃F₂N₄O₄ [M+H]⁺544.3, found 544.4.

Step 3: A solution of tert-butyl N-tert-butoxycarbonyl-N-[3-fluoro-4-[[5-[2-fluoro-4-(trideuteriomethyl) anilino]-4-methyl-3-pyridyl]methyl]-2-pyridyl]carbamate (300 mg, 551.86 umol) in HCl/MeOH (4 M, 5 mL) was stirred at 25° C. for 24 h. The mixture was concentrated. The residue was purified by flash chromatography on silica gel (Ethyl acetate in Petroleum ether=0-95%) to give 3-fluoro-4-[[5-[2-fluoro-4-(trideuteriomethyl) anilino]-4-methyl-3-pyridyl]methyl]pyridin-2-amine (100 mg, 291.22 μmol). LCMS Rₜ=0.394 min in 1 min chromatography, 5-95AB, ESI calcd. for C₁₉H₁₆D₃F₂N₂O₄ [M+H]⁺344.2, found 344.3.

Step 4: To a solution of 3-fluoro-4-[[5-[2-fluoro-4-(trideuteriomethyl) anilino]-4-methyl-3-pyridyl]methyl]pyridin-2-amine (90 mg, 262.09 μmol) and Py (103.66 mg, 1.31 mmol, 105.77 μL) in MeCN (1 mL) was added N-methylsulfamoyl chloride (50.94 mg, 393.15 μmol) under N₂. The mixture was stirred at 25° C. for 1 h. N-methylsulfamoyl chloride (67.92 mg, 524.21 μmol) was added. The mixture was stirred at 25° C. for 1 h. The mixture was concentrated. The residue was purified by prep-HPLC (column: Phenomenex C18 75*30 mm*3 um; mobile phase: [water(NH₃H₂O+NH₄HCO₃)−ACN]; gradient: 18%-48% B over 7 min) to give 3-fluoro-4-[[5-[2-fluoro-4-(trideuteriomethyl) anilino]-4-methyl-3-pyridyl]methyl]-N-(methylsulfamoyl)pyridin-2-amine (19.4 mg, 44.45 μmol). ¹H NMR (400 MHz, DMSO-d₆) δ=10.35 (br s, 1H), 8.08-7.88 (m, 3H), 7.32 (s, 1H), 7.03 (dd, J=2.0, 12.8 Hz, 2H), 6.87 (dd, J=1.6, 8.0 Hz, 1H), 6.73 (t, J=8.8 Hz, 2H), 4.06 (s, 2H), 2.50-2.47 (m, 3H), 2.06 (s, 3H). ¹⁹F NMR (376.5 MHz, DMSO-d₆) δ=−126.425, −138.254. LCMS Rₜ=0.454 min in 1 min chromatography, 5-95AB, ESI calcd. for C₂₀H₁₉D₃F₂N₅O_{2S} [M+H]⁺437.2, found 437.2. LCMS Rₜ=0.454 min in 1 min chromatography, 5-95AB, ESI calcd. for C₂₀H₁₉D₃F₂N₅O₂S [M+H]⁺437.2, found 437.2.

### Example 105: Cellular Assays

PERK: Detection of pERK Thr202/Tyr204

A549 cultured in F-12K/10% FBS were seeded at 10000 cells/well, and HCT116 cultured in McCoy's 5A/10% FBS were seeded at 15000 cells/well cells into Corning 384-well plates, respectively. Cells were incubated overnight in a TC incubator. Compounds were serially diluted and added to cells for 2 h in a TC incubator. Cells were then lysed according to manufacture protocol (Cisbio CAT #: 64AERPEG). Cell lysate was mixed with phospho-ERK (Thr202/Tyr204) antibody solution at 5:1 (v:v). Lysate and antibody mixture were incubated overnight at room temperature. HTRF signal was read at two different wavelengths (665 nm and 620 nm) on a compatible HTRF reader. The emission of light by the acceptor will be proportional to the level of interaction can be plotted as % inhibition values for tested compounds are plotted and the concentration of compound required for 50% inhibition (IC50) is determined with a four-parameter logistic dose response equation. The results are shown in Table 1 below.

Determination of pMEK IC_{50S}

A549 cells were cultured in F12K/10% FBS media (ATCC CAT #21127022) and were seeded at 10000 cells/well and HCT116 cells were cultured in McCoy's 5A/10% FBS media (Gibco Cat #30-2007) were seeded at 15000 cells/well in 384 microplates (Corning CAT #3765). Cells were incubated overnight in a TC incubator. Compounds were serially diluted and added to cells for 2 h in a TC incubator. At the end of the 2 h EGF (R&D Systems 236-EG-200) was added at a final EGF concentration of 30 ng/ml and incubated for 15 minutes at 37° C. in a TC incubator. AlphaLisa (Perkin Elmer) was performed according to manufacturer's instructions (Perkin Elmer CAT #ALPHA.SF ULTRA MEK1 PS218/222). The percent (%) inhibition at each concentration of compound is calculated based on and relative to the AlphaLISA signal in the HPE and ZPE control wells contained within each assay plate. The ZPE control wells contain cells and DMSO as 0% inhibition, and the HPE control wells only contain cells and control compound (Sellekchem Staurosporin CAT #S1421) as 100% inhibition. The concentrations and % inhibition values for tested compounds are plotted and the concentration of compound required for 50% inhibition (IC50) is determined with a four-parameter logistic dose response equation. The results are shown in Table 1 below.

### HCT116 and IPC298 Cell Titer Glow Assay (CTG)

HCT116 cells and IPC298 cells were diluted with culture medium to the desired density 500 cells/well and 40 μL of cell suspension were added into each well of 384-well cell culture plate. McCoy's 5A (Gibco Catalogue No. 16600108) plus 10% fetal bovine serum plus 1% pencillin/streptomycin was the culture medium used for HCT116 cells and RPMI1640 (ATCC Catalogue No. 30-2001) plus 10% fetal bovine serum plus 1% pencillin/streptomycin was the culture medium used for IPC298 cells. The plates were covered and stored at room temperature for 30 minutes without shaking and then the plates were transferred into a 37° C. 5% CO₂ incubator overnight.

A 10 mM solution in dimethyl sulfoxide (DMSO) was prepared for each test compound. A 3 fold, 10-point dilution was prepared from the stock solution. 40 nl of each solution in 100% DMSO was added to the 384-well plate and diluted to a final 0.1% DMSO final concentration. The plates were then incubated for 72 hours (for both cell lines) at 37° C.

CellTiter-Glo® 2.0 Reagent (Promega, G9242) was removed from the refrigerator and equilibrated at room temperature (23° C.) for 60 minutes. The plates were then removed from the incubator and allowed to equilibrate to room temperature. 30 μL CellTiter-Glo® 2.0 Reagent were added to the assay plates, and spun 1 min/1000 rpm. The plates were allowed to stand for about 20 minutes before being read on Envision using a standard luminescence readout. DMSO was employed as negative control (High control, HC) and 1 uM Staurosporine was employed as positive control (Low control, LC).

The percent (%) inhibition at each concentration of compound was calculated based on the signal in the negative and positive control wells contained within each assay plate. The concentrations and % inhibition values for tested compounds are were plotted and the concentration of compound required for 50% inhibition (IC50) was determined with a four-parameter logistic dose response equation.

$$\% growth = 100 \times [ ( X - day 0 ) / DMSO - day 0 ] .$$

The results are shown in Table 1 below.

TABLE 1
Cell assay data

| | | | | CTG | | CTG | CTG | | |
|---|---|---|---|---|---|---|---|---|---|
| | | | CTG | % cell | CTG | % cell | % cell | | |
| | IC₅₀ (nM) | IC₅₀ (nM) | IC₅₀ (nM) | viability | IC₅₀ (nM) | viability | viability | IC₅₀ (nM) | IC₅₀ (nM) |
| | A549.2 | HCT116.2 | Cell Line: | 1000 uM | Cell Line: | 1000 uM | 100 uM | A549.2 | HCT116.2 |
| Example | pMEK | pMEK | HCT116.20 | HCT116.20 | IPC298.2 | IPC298.2 | IPC298.2 | pERK | pERK |
| 1 | A | A | A | | A | | | A | A |
| 2 | | | | | | | | | |
| 3 | B | B | | | | | | A | A |
| 4 | B | B | | | | | | B | B |
| 5 | A | A | B | | A | | | A | A |
| 6 | A | A | B | | | | | A | A |
| 7 | A | A | | | | | | | |
| 8 | A | A | | | | | | | |
| 9 | A | B | | | | | | A | A |
| 10 | A | A | A | | A | | | A | A |
| 11 | B | A | | 31.79 | B | | | A | A |
| 12 | A | A | | | | | | A | A |
| 13 | A | A | | | | | | | |
| 14 | A | A | | | | | | | |
| 15 | A | A | B | | A | | | | |
| 16 | B | B | E | 36.55 | C | | | | |
| 17 | D | E | E | 68.21, | E | | 72.73; | | |
| | | | | 83.74 | | | 98.52 | | |
| 18 | A | A | C | | B | | | | |
| 19 | D | D | | | | | | | |
| 20 | E | E | | | | | | | |
| 21 | B | B | E | 76.96, | D | | | | |
| | | | | 35.15 | | | | | |
| 22 | A | A | C | | B | | | | |
| 23 | A | A | B | | | | | | |
| 24 | B | >100.000 | C | | A | | | | |
| 25 | E | E | | | | | | | |
| 26 | A | A | C | | B | | | | |
| 27 | A | A | D | | B | | | | |
| 28 | B | B | E | 50.42, | B | | | | |
| | | | | 16.68 | | | | | |
| 29 | B | B | | | | | | | |
| 30 | B | B | | | | | | | |
| 31 | E | E | | | | | | | |
| 32 | E | E | | | | | | | |
| 33 | A | A | A | | A | | | | |
| 34 | D | E | | | | | | | |
| 35 | A | A | A | | A | | | | |
| 36 | A | A | | | | | | | |
| 37 | A | A | B | | A | | | | 0.362 |
| 38 | A | A | A | | A | | | | 0.241 |
| 39 | A | A | B | | A | | | | 0.354 |
| 40 | E | E | E | 100.62 | E | | 95.15 | | >1000 |
| 41 | A | A | C | | B | | | | 2.317 |
| 42 | A | A | C | | B | | | | 0.791 |
| 43 | | | B | | A | | | | |
| 44 | | | E | 27.98 | B | | | | |
| 45 | | | B | | | | | | |
| 46 | | | E | 82.81, | E | 19.34 | | | |
| | | | | 79.54 | | | | | |
| 47 | | | A | | A | | | | |
| 48 | | | E | 17.49 | E | −6.55, | | | |
| | | | | | | 57.95 | | | |
| 49 | | | B | | A | | | | |
| 50 | | | D | | B | | | | |
| 51 | | | C | | | | | | |
| 52 | | | C | | A | | | | |
| 53 | | | B | | | | | | |
| 54 | | | E | 81.24 | E | 70.07 | | | |
| 55 | | | E | 80.92 | | | | | |
| 56 | | | C | | A | | | | |
| 57 | | | E | 56.78, | B | | | | |
| | | | | 45.51 | | | | | |
| 58 | | | A | | A | | | | |
| 59 | | | C | | B | | | | |
| 60 | | | C | | A | | | | |
| 61 | | | E | 93.19 | E | | 94.66 | | |
| 62 | | | E | 46.4 | B | | | | |
| 63 | | | E | 71.54 | E | | 62.36 | | |
| 64 | | | C | | A | | | | |
| 65 | | | B | | A | | | | |
| 66 | | | B | | A | | | | |
| 67 | | | B | | A | | | | |
| 68 | | | D | | E | | | | |
| 69 | | | E | 60.4 | B | | | | |
| 70 | | | E | 50.22 | B | | | | |
| 71 | | | A | | A | | | | |
| 72 | | | D | | B | | | | |
| 73 | | | E | 75.42 | E | | 80.82 | | |
| 74 | | | E | 92.91 | E | | 88.62 | | |
| 75 | | | E | 89.1 | E | | 87.52 | | |
| 76 | | | C | | A | | | | |
| 77 | | | C | | A | | | | |
| 78 | | | C | | A | | | | |
| 79 | | | A | | A | | | | |
| 80 | | | A | | A | | | | |
| 81 | | | C | | A | | | | |
| 82 | | | E | 93.03 | E | | 55.98 | | |
| 83 | | | B | | A | | | | |
| 84 | | | D | | A | | | | |
| 85 | | | E | 102.18 | E | | 106.29 | | |
| 86 | | | C | | B | | | | |
| 87 | | | E | 94.97 | E | | 100.53 | | |
| 88 | | | C | | A | | | | |
| 89 | | | E | 94.97 | E | | 100.16 | | |
| 90 | | | E | 59.50 | B | | | | |
| 91 | | | B | | A | | | | |
| 92 | | | E | 102.76 | E | | 101.21 | | |
| 93 | | | B | | A | | | | |
| 94 | | | C | | A | | | | |
| 95 | | | B | | A | | | | |
| 96 | | | B | | A | | | | |
| 97 | | | A | | A | | | | |
| 98 | | | C | | A | | | | |
| 99 | | | A | | A | | | | |
| 100 | | | B | | A | | | | |
| 101 | | | B | | A | | | | |
| 102 | | | B | | A | | | | |
| 103 | | | C | | A | | | | |
| 104 | | | A | | A | | | | |

A is an IC50 < 10 nM; B is 10-100 nM; C is 100-500 nM; D is 500 to 1000; and E is >1000 nM. Percent viability is provided for many of the compounds falling within the “E” range, which means percent cells remaining after treatment. Values under 100% are indicative of efficacy at inhibiting growth.

What is claimed is:

1. A compound represented by the following structural formula (I): 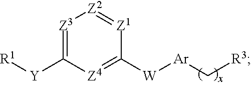
or a pharmaceutically acceptable salt thereof, wherein:
Y is a covalent bond, NH, NCH₃, S, CH₂, OCH₂\hat{ } or O, wherein “\hat{ }” indicates the point of attachment to R¹;
W is CH₂, CH(CH₃) or O;
Z¹, Z² and Z³ are each independently selected from N, N-oxide and CR^{2a}, provided that no more than one of Z¹, Z² and Z³ is an N-oxide;
Z⁴ is slected from N or CR^{2b}
Ar is phenyl, a six to membered heteroaryl or 2-pyridinone, wherein the phenyl, the six membered heteroaryl, and 2-pyridinone are each independently substituted with zero, one or two groups represented by R⁴ and wherein 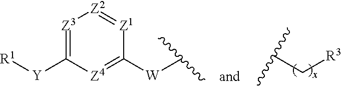
are 1,3 or 1,4 relative to each other on the group represented by Ar;
R¹ is, C₁₋₆ alkyl, C₂₋₆ alkenyl, C₂₋₆ alkynyl, C₁₋₆ haloalkyl, pyridinonyl, C₃₋₆ cycloalkyl, phenyl, a 5-10 membered heteroaryl or C(O)N(R⁶)₂, wherein the C₃₋₆ cycloalkyl, phenyl, and the 5-10 membered heteroaryl, are each independently substituted with zero, one, two or three groups represented by R⁵;
R^{2a} is H, F or C₁₋₃ alkyl;
R^{2b} is H, halo, (CH₂)ₙOR⁷, C₁₋₆ alkyl, C₂₋₆ alkenyl, C₂₋₆ alkynyl, C₁₋₆ haloalkyl, C₁₋₆ alkoxy, C(O)N(C₁₋₆-alkyl), C(O)NHO(C₂₋₆ hydroxyalkyl), (CH₂)₂₋₆N(R⁷)₂, C(O)NHO(CH₂)₂₋₆N(R⁷)₂; wherein each R²⁰ is H or C₁₋₆ alkyl, C₃₋₆ cycloalkyl, phenyl, a 5-6 membered heteroaryl or 4-6 membered heterocycle; or R^{2b} and Y taken together with their intervening atoms form a 5-6 membered nitrogen containing heterocycle or a 5-6 membered nitrogen containing heteroaryl; and
R³ is N(R¹⁰)₂, 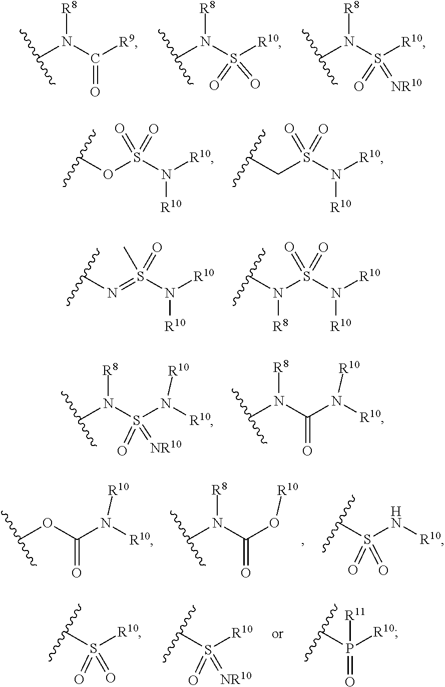
each R⁴ is independently H, halo, C₁₋₆ alkoxy or C₁₋₆ alkyl;
R⁵ is H, cyano, halo, SO₂ C₁₋₆ alkyl, C₁₋₆ alkyl, deuterated C₁₋₆ alkyl, C₂₋₆ alkenyl, deuterated C₁₋₆ alkenyl, C₂₋₆ alkynyl, deuterated C₁₋₆ alkynyl, C₁₋₆ haloalkyl, C₁₋₆ alkoxy, C₁₋₆ haloalkoxy, SC₁₋₆ alkyl, C₃-8 cycloalkyl; or two R⁵s on adjacent phenyl ring carbon atoms taken together with the ring carbon atoms to which they are attached form an oxygen containing heterocycle; or two R⁵s on the same ring carbon atom of a C₃₋₆ cycloalkyl form a 4-6 membered nitogen containing heterocycyle optionally substituted with C₁₋₄ alkyl; and
each R⁶ is independently selected from H or C₁₋₆ alkyl (preferably H or C₁₋₆ alkyl);
each R⁷ and each R⁸ are independently selected from H or C₁₋₃ alkyl; or when x is 0, R⁸ and an R⁴ ortho to W and R³ taken together with their intervening atoms form a 5-6 membered nitrogen containing heterocycle;
R⁹ is H, C₁₋₆ alkoxy, C₁₋₆ alkyl, C₂₋₆ alkenyl, C₁₋₆ haloalkyl, C₃₋₈ cycloalkyl (optionally substituted with methyl) or N(R¹¹)₂ wherein the C₁₋₆ alkyl is optionally substituted with cyano, hydroxy, C₁₋₆ alkoxy or N(R¹¹)₂;
each R¹⁰ is independently H, C₁₋₆ alkyl, C₂₋₆ alkenyl, C₃₋₈ cycloalkyl (optionally substituted with methyl) or C₁₋₆ haloalkyl, wherein the C₁₋₆ alkyl is optionally substituted with cyano, hydroxy, C₁₋₆ alkoxy or N(R¹¹)₂; or
two R¹⁰s taken together with the nitrogen atom to which they are bonded form a 3-7 membered heterocycle;
each R¹¹ is independently H or methyl;
n is 0 or 1; and
x is 0 or 1.

2. The compound of claim 1, represented by the following structural formula: 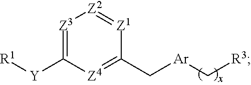
or a pharmaceutically acceptable salt thereof, wherein:
Y is a covalent bond, NH, NCH₃, S, CH₂, OCH₂\hat{ } or O, wherein “\hat{ }” indicates the point of attachment to R¹;
Z¹, Z² and Z³ are each independently selected from N and CR^{2a};
Z⁴ is slected from N or CR^{2b}
Ar is phenyl, a six membered heteroaryl or 2-pyridinone, wherein the phenyl, the six membered heteroaryl, and 2-pyridinone are each independently substituted with zero, one or two groups represented by R⁴ and wherein 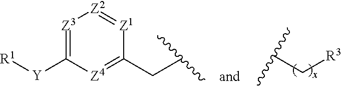
are 1,3 or 1,4 relative to each other on the group represented by Ar;
R¹ is, C₁₋₆ alkyl, C₂₋₆ alkenyl, C₂₋₆ alkynyl, C₁₋₆ haloalkyl, C₃-6 cycloalkyl, phenyl, a 5-6 membered heteroaryl or C(O)N(R⁶)₂, wherein the phenyl, and the 5-6 membered heteroaryl, are each independently substituted with zero, one or two groups represented by R⁵;
R^{2a} is H, F or C₁₋₃ alkyl;
R^{2b} is H, halo, (CH₂)ₙOR⁷, C₁₋₆ alkyl, C₂₋₆ alkenyl, C₂₋₆ alkynyl, C₁₋₆ haloalkyl, C₁₋₆ alkoxy, C₃₋₆ cycloalkyl, phenyl, a 5-6 membered heteroaryl or 4-6 membered heterocycle;
R³ is N(R¹⁰)₂, 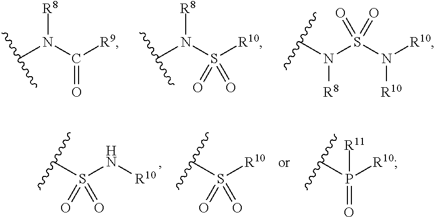
each R⁴ is independently H, halo, C₁₋₆ alkoxy or C₁₋₆ alkyl;
R⁵ is H, cyano, halo, C₁₋₆ alkyl, C₂₋₆ alkenyl, C₂₋₆ alkynyl, C₁₋₆ haloalkyl, C₁₋₆ alkoxy, halomethoxy or C₃₋₈ cycloalkyl;
each R⁶ is independently selected from H or C₁₋₆ alkyl;
R⁷ and R⁸ are independently selected from H or C₁₋₃ alkyl;
R⁹ is H, C₁₋₆ alkoxy, C₁₋₆ alkyl, C₂₋₆ alkenyl, C₁₋₆ haloalkyl, C₃₋₈ cycloalkyl (optionally substituted with methyl) or N(R¹¹)₂ wherein the C₁₋₆ alkyl is optionally substituted with cyano, hydroxy, C₁₋₆ alkoxy or N(R¹¹)₂;
each R¹⁰ is independently H, C₁₋₆ alkyl, C₂₋₆ alkenyl, C₃₋₈ cycloalkyl (optionally substituted with methyl) or C₁₋₆ haloalkyl, wherein the C₁₋₆ alkyl is optionally substituted with cyano, hydroxy, C₁₋₆ alkoxy or N(R¹¹)₂; or
two R¹⁰s taken together with the nitrogen atom to which they are bonded form a 3-7 membered heterocycle;
each R¹¹ is independently H or methyl;
n is 0 or 1; and
x is 0 or 1.

3. The compound of claim 1, or pharmaceutically acceptable salt thereof, wherein R¹ is C₁₋₄ alkyl, C₂₌₄ alkenyl, C₂₋₄ alkynyl, 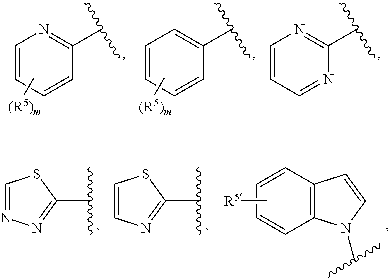
cycloalkyl optionally substituted with one or two R⁵″ or C(O)N(R⁶)₂; R⁵′ is H or halo; each R⁵″ is C₁₋₃ alkyl or two R⁵″ taken together with the ring carbon atom to which they are bonded form a C₄₋₆ nitrogen containing heterocyclyl wherein the ring nitrogen atom is optionally N—(C₁₋₃) alkylated; and m is 0, 1 or 2.

4. The compound of claim 2, or pharmaceutically acceptable salt thereof, wherein R¹ is, 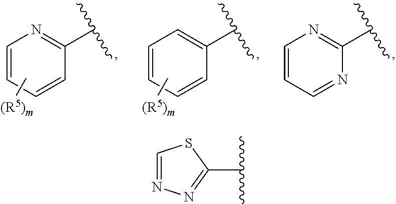
or C(O)N(R⁶)₂, and m is 0, 1 or 2.

5. The compound of any one of claims 1-4, or a pharmaceutically acceptable salt thereof, represented by a structural formula selected from:

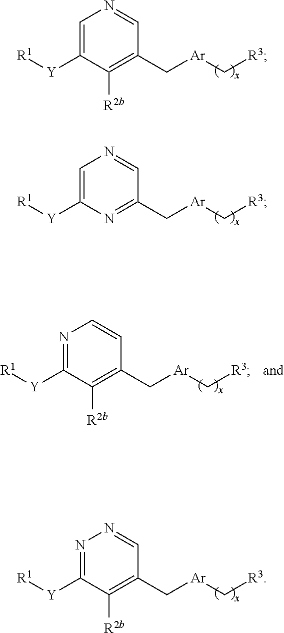

6. The compound of claim 2 or 4, represented by the following structural formula: 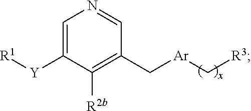
or a pharmaceutically acceptable salt thereof.

7. The compound of any one of claim 1, 3 or 5-6, or a pharmaceutically acceptable salt thereof, wherein:
R¹ is methyl, allyl, propargyl, 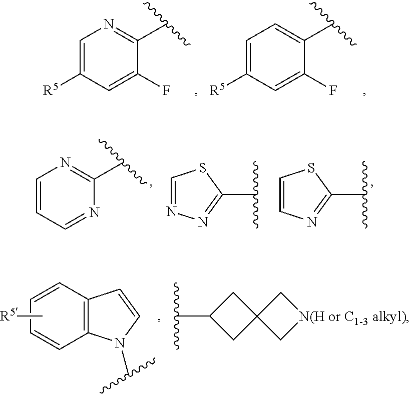
cyclohexyl optionally substituted by one or two methyl or C(O)N(R⁶)₂.

8. The compound of any one of claim 2, 4 or 6, or a pharmaceutically acceptable salt thereof, wherein: 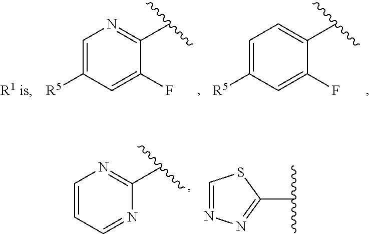
or C(O)N(R⁶)₂.

9. The compound of any one of claims 1-8, or a pharmaceutically acceptable salt thereof, wherein Ar—(CH₂)ₓ—R³ is represented by the following structural formula: 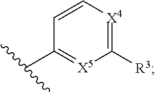
wherein X⁴ is N, CH, C(C₁₋₄alkyl) or C(C₁₋₄alkoxy) and X⁵ is N or CR⁴.

10. The compound of any one of claim 2, 4 or 8, or a pharmaceutically acceptable salt thereof, represented by the following structural formula: 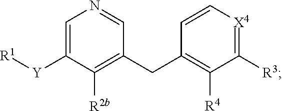
wherein X⁴ is N or CH.

11. The compound of any one of claim 2, 4 or 8, or a pharmaceutically acceptable salt thereof, represented by the following structural formula:

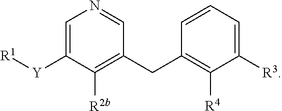

12. The compound of any one of claim 2, 4 or 8, or a pharmaceutically acceptable salt thereof, represented by the following structural formula:

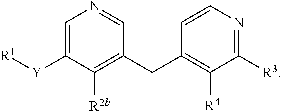

13. The compound of any one of claim 2, 4 or 8, or a pharmaceutically acceptable salt thereof, represented by the following structural formula:

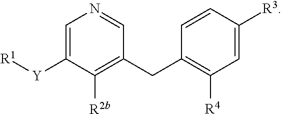

14. The compound of any one of claims 1-9, or a pharmaceutically acceptable salt thereof, wherein —Ar—(CH₂)ₓR³ is represented by a structural formula selected from:

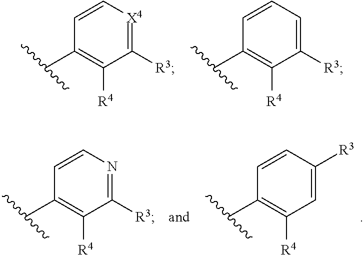

15. The compound of any one of claim 1-14, or a pharmaceutically acceptable salt thereof, wherein R¹ is

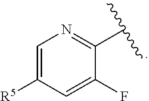

16. The compound of any one of claim 1-14, or a pharmaceutically acceptable salt thereof, wherein R¹ is

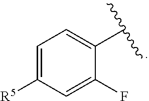

17. The compound of any one of claim 1-14, or a pharmaceutically acceptable salt thereof, wherein R¹ is

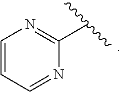 [SMILES: *c1ncccn1]

18. The compound of any one of claim 1-14, or a pharmaceutically acceptable salt thereof, wherein R¹ is

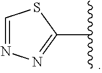 [SMILES: *c1nncs1]

19. The compound of any one of claim 1-14, or a pharmaceutically acceptable salt thereof, wherein R¹ is C(O)N(R⁶)₂, wherein R⁶ is H or C₁₋₆ alkyl, preferably H or methyl.

20. The compound of any one of claims 1-19, or a pharmaceutically acceptable salt thereof, wherein x is 0 and R³ is

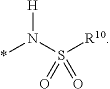

21. The compound of any one of claims 1-19, or pharmaceutically acceptable salt thereof, wherein x is 0 and R³ is

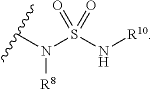

22. The compound of any one of claims 1-19, or a pharmaceutically acceptable salt thereof, wherein x is 0 and R³ is

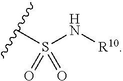

23. The compound of any one of claims 1-19, or a pharmaceutically acceptable salt thereof, wherein x is 0 and R³ is

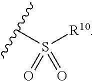

24. The compound of any one of claims 1-19, or a pharmaceutically acceptable salt thereof, x is 0 and R³ is

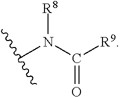

25. The compound of any one of claim 2, 4, 6, 8 or 10-13, or a pharmaceutically acceptable salt thereof, wherein Y is O.

26. The compound of any one of claim 2, 4, 6, 8 or 10-13, or a pharmaceutically acceptable salt thereof, wherein Y is NH.

27. The compound of any of claims 1 to 24, or a pharmaceutically acceptable salt thereof, wherein Y is O, NH, N(CH₃) or S.

28. The compound of claim 2, 4, 6, 8, 10-13 or 15-24, or a pharmaceutically acceptable salt thereof, wherein R⁸ is H, R⁹ is C₁₋₆ alkoxy, C₁₋₆ alkyl, or N(R¹¹)₂ and R¹⁰ is C₁-C₆ alkyl.

29. The compound of any of claims 1 to 28, or a pharmaceutically acceptable salt thereof, wherein R^{2b} is H, C₁₋₆ alkyl, halo, C₁₋₆ alkoxy, (CH₂)ₙOR⁷ or 4-6 membered heterocycle; R⁴ is H, C₁₋₆ alkoxy or halo; and R⁵ is H, C₁₋₆ alkyl, deuterated C₁₋₆ alkyl, C₁₋₆ alkynyl, cyano, C₁₋₆ haloalkyl, C₁₋₆ alkoxy, C₁₋₆ haloalkoxy, SO₂ C₁₋₆ alkyl, SC₁₋₆ alkyl, halo or C₃₋₈ cycloalkyl.

30. The compound of claim 2, 4, 6, 8, 10-13, 15-26 or 28, or a pharmaceutically acceptable salt thereof, wherein R^{2b} is C₁₋₆ alkyl, halo, C₁₋₆ alkoxy, (CH₂)ₙOR⁷ or 4-6 membered heterocycle; R⁴ is H or halo and R⁵ is H, C₁₋₆ alkyl, cyano, C₁₋₆ haloalkyl, halo or C₃₋₈ cycloalkyl.

31. The compound of any of claims 1 to 30, or a pharmaceutically acceptable salt thereof, wherein, R^{2b} is H, methyl, ethyl, chloro, OCH₃, CH₂OCH₃ or oxetane, R⁴ is H, OCH₃ or fluoro, R⁵ is H, fluoro, chloro, bromo, iodo, cyano, OCH₃, SCH₃, SO₂CH₃, CHF₂, CF₃, methyl, ethyl, iso-propropyl, iso-butyl, CD₃, C≡CH, OCF₃, OCHF₂ or cyclopropyl or two R⁵ groups on adjacent phenyl ring atoms form OCH₂CH₂O; and R⁶ is H or methyl.

32. The compound of any of claims 1 to 31, or a pharmaceutically acceptable salt thereof, wherein, R^{2b} is methyl, chloro, OMe, CH₂OCH₃ or oxetane, R⁴ is H or fluoro, R⁵ is H, fluoro, chloro, bromo, cyano, CF₃, methyl, ethyl, or cyclopropyl and R⁶ is H or methyl.

33. The compound of any of claims 1 to 32, or a pharmaceutically acceptable salt thereof, wherein R⁷ is H or methyl, R⁹ is OCH₃, methyl, or NHCH₃ and R¹⁰ is H, methyl, ethyl or propyl.

34. The compound of claim 2, 4, 6, 8, 10-13, 15-26, 28, 30 or 32, or a pharmaceutically acceptable salt thereof, wherein R⁷ is H or methyl, R⁹ is OCH₃, methyl, or NHCH₃ and R¹⁰ is methyl.

35. A pharmaceutical composition comprising a pharmaceutically acceptable carrier or diluent and the compound of claim any one of claims 1-34, or a pharmaceutically acceptable salt thereof.

36. A method of inhibiting mitogen-activated protein kinase (MEK) in a subject in need thereof, comprising administering an effective amount of: i) the compound of any one of claims 1-34, or a pharmaceutically acceptable salt thereof; or ii) the pharmaceutical composition of claim 35.

37. A method of treating a subject with cancer, comprising administering an effective amount of: i) the compound of any one of claims 1-34, or a pharmaceutically acceptable salt thereof; or ii) the pharmaceutical composition of claim 35.
